(19) United States
(12) Patent Application Publication
(10) Pub. No.: US 2010/0021424 A1
(43) Pub. Date: Jan. 28, 2010

(54) Method For Identifying Whether A Patient Will Be Responder or Not to Immunotherapy

(76) Inventors: Vincent Brichard, Rixensart (BE); James Scott Clark, Rixensart (BE); Thierry Coche, Rixensart (BE); Swann Romain Jean-Thomas Gaulis, Rixensart (BE); Olivier Gruselle, Rixensart (BE); Frederic Lehmann, Rixensart (BE); Jamila Louahed, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/303,213

(22) PCT Filed: May 31, 2007
(86) PCT No.: PCT/EP2007/004915

§ 371 Date: Dec. 2, 2008

(30) Foreign Application Priority Data
Jun. 2, 2006 (GB) 0610949.0
Jan. 15, 2007 (GB) 0700761.0

(51) Int. Cl. A61K 38/21; C12Q 1/68; A61P 35/00; A61N 5/10

(52) U.S. Cl. 424/85.4; 435/6; 378/65

(57) ABSTRACT

The present invention relates to gene expression profiles, microarrays comprising nucleic acid sequences representing gene expression profiles and new diagnostic kits and methods. The invention further relates to treatment of specific populations of, for example, cancer patients, as characterised by their gene expression profile, suffering from Mage expressing tumours.

### FIELD OF THE INVENTION

The present invention relates to gene expression profiles; microarrays comprising nucleic acid sequences for identifying said profiles, for example, by analysing patient derived samples; new diagnostic kits and methods for identification of same. The invention further relates to treatment of specific populations of patients, for example cancer patients, characterised as a responder by their gene expression profile, such as patients suffering from Mage expressing tumours. The invention further includes methods of inducing a responder's profile in a patient initially or originally designated as a non-responder.

### BACKGROUND

Melanomas are tumors originating from melanocyte cells in the epidermis. Patients with malignant melanoma in distant metastasis (stage IV according to the American Joint Commission on Cancer (AJCC) classification) have a median survival time of one year, with a long-term survival rate of only 5%. Even the standard chemotherapy for stage IV melanoma has therapeutic response rates of only 8-25%, but with no effect on overall survival. Patients with regional metastases (stage III) have a median survival of two to three years with very low chance of long-term survival, even after an adequate surgical control of the primary and regional metastases (Balch et al., 1992). Most Patients with stage I to III melanoma have their tumour removed surgically, but these patients maintain a substantial risk of relapse. Thus there remains a need to prevent melanoma progression, and to have improved treatment regimes for metastatic melanoma and adjuvant treatments for patients having had a primary tumour removed.

There are two types of lung cancer: non-small cell lung cancer (NSCLC) and small cell lung cancer (SCLC). The names simply describe the type of cell found in the tumours. NSCLC includes squamous-cell carcinoma, adenocarcinoma, and large-cell carcinoma and accounts for around 80% of lung cancers. NSCLC is hard to cure and treatments available tend to have the aim of prolonging life, as far as possible, and relieving symptoms of disease. NSCLC is the most common type of lung cancer and is associated with poor outcomes (Gatzmeier et al., 1994). Of all NSCLC patients, only about 25% have loco-regional disease at the time of diagnosis and are still amenable to surgical excision (stages IB, IIA or IIB according to the AJCC classification). However, more than 50% of these patients will relapse within the two years following the complete surgical resection. There is therefore a need to provide better treatment for these patients.

Traditional chemotherapy is based on administering toxic substances to the patient and relying, in part, on the aggressive uptake of the toxic agent by the tumour/cancer cells. These toxic substances adversely affect the patient's immune system, leaving the individual physically weakened and susceptible to infection.

It is known that not all patients with cancer respond to current cancer treatments. It is thought that only 30% or less of persons suffering from a cancer will respond to any given treatment. The cancers that do not respond to treatment are described as resistant. In many instances there have not been reliable methods for establishing if the patients will respond to treatment. However, administering treatment to patients who are both responders and non-responders because they cannot be differentiated is an inefficient use of resources and, even worse, can be damaging to the patient because, as discussed already, many cancer treatments have significant side effects, such as severe immunosuppression, emesis and/or alopecia. It is thought that in a number of cases patients receive treatment, when it is not necessary or when it will not be effective.

Cells including cancer/tumour cells express many hundreds even thousands of genes.

A large amount of work has been done in recent times to assist in the diagnosis and prognosis of cancer patients, for example to identify those patients who do not require further treatment because they have no risk of metastasis, recurrence or progression of the disease.

WO 2006/124836 identifies certain gene expression signatures over several oncogenic pathways, thereby defining the prognosis of the patient and sensitivity to therapeutic agents that target these pathways. The specific oncogenes are; Myc, Ras, E2, S3, Src and beta-catenin.

US 2006/0265138 discloses a method of generating a genetic profile, generally for identifying the primary tumour so that appropriate treatment can be given.

US 2006/0240441 and US 2006/0252057 describe methods of diagnosing lung cancer based on the differential expression of certain genes.

US 2006/0234259 relates to the identification and use of certain gene expression profiles of relevance to prostate cancer.

WO 2006/103442 describes gene expression profiles expressed in a subset of estrogen receptor (ER) positive tumours, which act, as a predictive signature for response to certain hormone therapies such as tamoxifen and also certain chemotherapies.

WO 2006/093507 describes a gene profile useful for characterising a patient with colorectal cancer as having a good prognosis or a bad prognosis, wherein patients with a good prognosis are suitable for chemotherapy.

WO 2006/092610 describes a method for monitoring melanoma progression based on differential expression of certain genes and novel markers for the disease, in particular TSBY1, CYBA and MT2A.

WO 2005/049829 describes an isolated set of marker genes that may be employed to predict the sensitivity of certain cancers to a chemotherapeutic agent, which is an erbB receptor kinase inhibitor, such as gefitinib.

Generally, these cases relate to markers for one or more cancers based on biological markers for the identification and/or progression of the cancer. In some instances these pathways are modulated by so-called oncogenes. Diagnosis employing the above techniques allows those patients who are likely to relapse and/or suffer metastasis to be identified and targeted for further therapy. In other instances a specific marker relevant to resistance to a specific treatment is identified.

A new generation of cancer treatments based on antigens, peptides, DNA and the like is currently under investigation by a number of groups. The strategy behind many of these therapies, often referred to as cancer immunotherapy, is to stimulate the patient's immune system into fighting the cancer. These therapies are likely to be advantageous because the side effects, of taking such treatments, are expected to be minimal in comparison to the side effects currently encountered by patients undergoing cancer treatment. An antigen used in a cancer immunotherapy may be referred to as an ASCI, that is antigen-specific cancer immunotherapeutic.

In the early 1980s, Van Pel and Boon published the discovery of cytolytic T cells directed against an antigen presented on tumour cells. This led to the characterization of the first tumour-specific, shared antigen: Melanoma AGE-1 (MAGE-1, subsequently renamed MAGE-A1). It was followed by the identification of a large number of genes sharing the same expression pattern: they are expressed in a wide range of tumour types such as, melanoma, lung, bladder, breast, head and neck cancers. They are not expressed in normal cells, except testis. However, this expression in the testis does not normally lead to antigen expression, as these germ line cells do not express MHC class I molecules. From their peculiar expression profile, the name of Cancer Testis (CT) genes was proposed for these genes.

MAGE antigens are antigens encoded by the family of Melanoma-associated antigen genes (MAGE). MAGE genes are predominately expressed on melanoma cells (including malignant melanoma) and some other cancers including NSCLC (non small cell lung cancer), head and neck squamous cell carcinoma, bladder transitional cell carcinoma and oesophagus carcinoma, but are not detectable on normal tissues except in the testis and the placenta (Gaugler et al Human gene MAGE-3 codes for an antigen recognized on a melanoma by autologous cytolytic T lymphocytes J Exp Med. 1994 Mar. 1; 179(3):921-930); Weynants et al Expression of mage genes by non-small-cell lung carcinomas Int. J. Cancer. 1994 Mar. 15; 56(6):826-829, Patard et al Int J. Cancer 64: 60, 1995). MAGE-A3 is expressed in 69% of melanomas (Gaugler, 1994), and can also be detected in 44% of NSCLC (Yoshimatsu 1988), 48% of head and neck squamous cell carcinoma, 34% of bladder transitional cell carcinoma, 57% of oesophageal carcinoma, 32% of colon cancers and 24% of breast cancers (Van Pel, et al Genes coding for tumor antigens recognized by cytolytic T lymphocytes Immunological Reviews 145, 229-250, 1995, 1995.); Inoue 1995; Fujie 1997; Nishimura 1997). Cancers expressing MAGE proteins are known as Mage associated tumours.

### SUMMARY

In one aspect the invention provides a method for detection of a gene signature, indicative of a responder or non-responder to immunotherapy, such as cancer immunotherapy, in a biological sample. Thus the invention provides a method of screening patients to establish if they are suitable for treatment, for example with a cancer immunotherapy.

In one aspect the invention provides a diagnostic kit comprising one or more nucleotide probes capable of hybridising to the mRNA or cDNA of one or more immune activation genes relevant to the profile.

In one aspect the invention provides one or more probes for identifying said one or more immune activation genes relevant to the profile.

In one aspect the invention provides a microarray of said probes suitable for the detection of said gene(s)/profile.

In another aspect the invention provides use of a microarray, including known microarrays, for the identification of said immune gene(s)/profile.

In one aspect the invention provides use of PCR (or other known techniques) for identification of differential expression (such as upregulation) of one or more of said genes.

In one aspect the invention provides a method of treating a patient with an appropriate immunotherapy after screening to identify the patient as a responder to said treatment.

In another aspect the invention provides a method of increasing the efficacy of an immunotherapy in a patient population comprising the step of first screening the population for the differential expression of one or more immune genes/said profile, and a second step of characterising the patient as a responder or non-responder.

In a further aspect the invention provides a method of generating a list of differentially expressed immune genes (ie a profile of immune genes) indicative of a responder or non-responder to immunotherapy.

### BRIEF DESCRIPTION OF THE FIGURES, SEQUENCES & TABLES

FIG. 1 is a diagrammatic representation of hierarchical clustering using Spotfire analysis linking the clinical outcome of 31 patients found to be responders (defined herein to include responder, mixed responder and stable disease) or non-responders, to Mage immunotherapy in the MAGE008 clinical trial, with the gene profile identified by microarray analysis and employing the 148 top probe sets.

FIG. 2 is a diagrammatic representation of the same analysis as FIG. 1, wherein the hierarchical clustering was performed using the BaldiBH analysis using 100 probes sets.

FIG. 3 is a diagrammatic representation of the same analysis as FIG. 1, wherein the hierarchical clustering was performed using Arrayminer Classmaker gene list.

FIG. 4 is a Venn diagram representing the comparison of gene lists used in FIG. 1, FIG. 2 and FIG. 3.

FIG. 5 is a visual representation of the expression profile of various genes (36 probe sets) for 30 different patients (patients are along the X-axis and the various probe sets extend along the Y-axis).

FIG. 5a is a visual representation of the expression of 2 genes for 30 different patients (patients along the X-axis).

FIG. 6 shows the Principal Component Analysis using PRF1, GZMB, GNLY, CD8A, PRKCQ, FOXP3, IFNG, CCL5, GPR171 and TRBV19 genes.

FIG. 7 is a visual representation of the expression profile of various genes (41 probe sets) for 33 different patients (patient identification numbers are along the X-axis) from the MAGE008 clinical trial using the adjuvant AS15.

FIG. 8 is a diagrammatic representation of hierarchical clustering for patients in the AS15 arm of the MAGE008 clinical trial.

FIG. 9 is a visual representation of the expression profile of various genes (41 probe sets) for 33 different patients (patient identification numbers are along the X-axis) from the MAGE008 clinical trial using the adjuvant AS02b.

FIG. 10 is a diagrammatic representation of hierarchical clustering for patients in the AS02b arm of the MAGE008 clinical trial.

In the heat maps herein upregulated genes are represented in red which in greyscale tends to be presented by darker shades. Lighter shades in greyscale tend to present green on the heat map (genes which are not upregulated).
Seq ID No 1 Chromosome 6 open reading frame 190
Seq ID No 2 Hematopoietic cell-specific Lyn substrate 1
Seq ID No 3 Interleukin 2 receptor, gamma (severe combined immunodeficiency)
Seq ID No 4 CD52 antigen (CAMPATH-1 antigen) /// CD52 antigen (CAMPATH-1 antigen)
Seq ID No 5 CD2 antigen (p50), sheep red blood cell receptor /// CD2 antigen (p50), sheep red blood cell receptor
Seq ID No 6 Ubiquitin D
Seq ID No 7 Signal transducer and activator of transcription 4
Seq ID No 8 Granzyme K (granzyme 3; tryptase 11) /// granzyme K (granzyne 3; tryptase II)
Seq ID No 9 CD3G antigen, gamma polypeptide (TiT3 complex)
Seq ID No 10 G protein-coupled receptor 171
Seq ID No 11 Protein kinase C, beta 1
Seq ID No 12 Major histocompatibility complex, class II, DR alpha /// major histocompatibility complex, class II, DR alpha
Seq ID No 13 Major histocompatibility complex, class II, DQ beta /// Major histocompatibility complex, class II, DQ beta 1
Seq ID No 14 Alcohol dehydrogenase IB (class I), beta polypeptide
Seq ID No 15 T cell receptor alpha constant /// T cell receptor alpha constant
Seq ID No 16 CD69 antigen (p60, early T-cell activation antigen)
Seq ID No 17 Protein kinase C, theta
Seq ID No 18 T cell receptor beta variable 19 /// T cell receptor beta constant 1
Seq ID No 19 T cell receptor alpha locus /// T cell receptor delta variable 2 μl T cell receptor alpha variable 20 /// T cell receptor alpha joining 17 /// T cell receptor alpha constant
Seq ID No 20 T cell receptor gamma constant 2
Seq ID No 21 T cell receptor beta variable 21-1 /// T cell receptor beta variable 19 /// T cell receptor beta variable 5-4 μl T cell receptor beta variable 3-1 /// T cell receptor beta constant 1
Seq ID No 22 T cell receptor alpha locus
Seq ID No 23 T cell receptor beta variable 19 /// T cell receptor beta variable 19 /// T cell receptor beta constant 1 /// T cell receptor beta constant 1
Seq ID No 24 CD3D antigen, delta polypeptide (TiT3 complex)
Seq ID No 25 T cell receptor gamma constant 2 /// T cell receptor gamma variable 9 /// similar to T-cell receptor gamma chain C region PT-gamma-1/2 /// similar to T-cell receptor gamma chain V region PT-gamma-1/2 precursor /// TCR gamma alternate reading frame protein
Seq ID No 26 Pyrin and HIN domain family, member 1
Seq ID No 27 T cell receptor associated transmembrane adaptor 1
Seq ID No 28 SLAM family member 7
Seq ID No 29 Chromosome 4 open reading frame 7
Seq ID No 30 Major histocompatibility complex, class II, DQ alpha 1
Seq ID No 31 Transcribed locus
Seq ID No 32 Amphiphysin (Stiff-Man syndrome with breast cancer 128 kDa auto antigen)
Seq ID No 33 Dendritic cell-associated lectin-1
   Each sequences above is given in Table 1A below.
Seq ID No 34 Nucleotide sequence encoding fusion protein of Lipoprotein D fragment, Mage3 fragment, and histidine tail
Seq ID No 35 Amino acid sequence of the fusion protein of Lipoprotein D fragment, Mage3 fragment and histidine tails
Seq ID No.s 36 to 43 (found in the description) are peptide sequences relevant to MAGE A3.
Seq ID No.s 44 to 48 are examples of oliogionucleotide sequences containing a CpG motif.
Seq ID No.s 49 to 84 (listed in Table 1B) are probes sets suitable for hybridising to the genes listed in Table 1.
Seq ID No.s 85 to 97 (listed in Table 3A) are sequences for the genes listed in Table 3.

Tables 1 to 4, 11 and 12 provide lists of genes that are differentially regulated according to the present invention.

Table 1A provides the nucleotide sequence listing for each of the genes listed in Table 1.

Table 1B provides the probe set identifier number (a unique reference number for the probe) of probes (and sequence thereof) wherein each probe is suitable for identifying particular genes listed in Table 1.

Table 3A links the genes (and sequences thereof) of Table 3 and probes suitable for identifying said genes.

Table 5 provides a list of genes, primers and probes suitable for use in PCR analysis.

Table 6 is a list of gene included in the TaqMan® (Q-PCR) Immune Profiling Array.

Table 7 provides a list of genes according to one aspect of the invention.

Table 7A provides the geomean ratio between responders and non-responder groups for the genes listed in Table 7.

Table 8 provides a correlation matrix for 30 genes.

Table 9 provides a list of genes according to one aspect of the invention.

Table 9A logistical regression results for certain genes listed in Table 9

Table 10 shows the percentage of correct classification using a logistical regression model for the genes listed in Table 9.

Table 11 provides a list of genes according to one aspect of the invention.

Tables 11A & 11B show the level of gene expression (based on the results of 41 probe sets) for various patients.

Tables 12 and 13 show gene lists that form further aspects of the invention.

Table 14 provides a correlation between the gene expression levels given in Tables 11A & 11B with the clinical outcome for said patients.

Annexes A to C are computer code for assisting with stastical analysis of samples.

The MAGE-1 gene belongs to a family of 12 closely related genes, MACE 1, MAGE 2, MAGE 3, MAGE 4, MAGE 5, MAGE 6, MAGE 7, MAGE 8, MAGE 9, MAGE 10, MAGE 11, MAGE 12, located on chromosome X and sharing with each other 64 to 85% homology in their coding sequence (De Plaen, 1994). These are sometimes known as MAGE A1, MAGE A2, MAGE A3, MAGE A4, MAGE A5, MAGE A6, MAGE A7, MAGE A8, MAGE A9, MAGE A 10, MAGE A11, MAGE A12 (The MAGE A family).

Two other groups of proteins are also part of the MAGE family although more distantly related. These are the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the Mage C family currently includes MAGE C1 and MAGE C2.

In general terms, a MAGE A protein can be defined as containing a core sequence signature located towards the C-terminal end of the protein (for example with respect to MAGE A1 a 309 amino acid protein, the core signature corresponds to amino acid 195-279).

It does not simply follow that if the tumour expresses, for example, Mage that the patient will respond to immunotherapy based on a Mage antigen.

### STATEMENT OF INVENTION

Analysis performed on cancers/tumours from patients prior to receiving immunotherapy, such as Mage immunotherapy, identified that certain genes were differentially expressed in patients that responded well to the treatment, in comparison to those patients who did not respond to the immunotherapy. The present inventors have discovered a gene signature/profile that is predictive of the likely response of the patient to an appropriate immunotherapy. More specifically the present inventors have discovered a gene signature that is predictive of improved survival after treatment with Mage antigen specific immunotherapy.

Thus the invention provides a gene profile, from a patient derived sample, which is indicative of an increased likelihood that the patient will be a responder (or alternatively a non-responder) to an immunotherapy, for example a cancer immunotherapy, such as Mage immunotherapy, wherein the profile comprises differential expression of at least one immune activation gene. The invention provides a gene profile, from a patient derived sample, which is indicative of an increased likelihood that the patient will be a responder to an immunotherapy, for example a cancer immunotherapy, such as Mage immunotherapy, wherein the profile comprises upregulation of at least one immune activation gene.

The present invention provides a predictive profile in contrast to a diagnostic or prognostic profile.

Whilst not wishing to be bound by theory it is hypothesised that the gene signature identified is in fact indicative of an immune/inflammatory, such as a T cell infiltration/activation response in the patients who are designated as responders, for example, the signature may represent a T-cell activation marker. The presence of this response is thought to assist the patient's body to fight the disease, such as cancer, after administration of the immunotherapy thereby rendering a patient more responsive to said immunotherapy.

Thus the signature of the present invention does not focus on markers/genes specifically associated with the diagnosis and/or prognosis of the relevant disease, for example cancer such as oncogenes, but rather is predictive of whether the patient will respond to an appropriate immunotherapy, such as cancer immunotherpay.

The gene profile identified herein for cancer and methods for identifying the same are thought to be indicative of the microenvironment of the tumor. The correct microenvironment of the tumor seems to be key to whether the patient responds to appropriate cancer immunotherapy. Immunotherapy in the context of the invention means therapy based on stimulating an immune response, generally to an antigen, wherein the response results in the treatment, amelioration and/or retardation of the progression of a disease associated therewith. Treatment in this context would not usually include prophylactic treatment.

Cancer immunotherapy in the context of this specification means immunotherapy for the treatment of cancer. In one aspect the immunotherapy is based on a cancer testis antigen, such as Mage (discussed in more detail below).

This invention may be used for identifying cancer patients that are likely to respond to appropriate immunotherapy, for example patients with melanoma, breast, bladder, lung, NSCLC, head and neck cancer, squamous cell carcinoma, colon carcinoma and oesophageal carcinoma, such as in patients with MAGE-expressing cancers. In an embodiment, the invention may be used in an adjuvant (post-operative, for example disease-free) setting in such cancers, particularly lung and melanoma. The invention also finds utility in the treatment of cancers in the metastatic setting.

Thus in a first aspect the invention provides a signature indicative of a patient, such as a cancer patient, designated a responder or non-responder to treatment with an appropriate immunotherapy, the signature comprising differential expression of one or more genes selected from immune activation/immune response/inflammatory response genes, for example, the group of genes indicative of T cell infiltration/activation, such as a gene listed (or a gene list comprising or consisting) those listed in Table 1, 2, 3, 4, 7, 9, 11, 12 and/or 13 such as Table 1 or 2. Differential expression in the context of the present invention means the gene is upregulated or downregulated in comparison to its normal expression. Statistical methods for calculating gene differentiation are discussed below.

Immune activation gene is intended to mean a gene that facilitates, increases or stimulates an appropriate immune response. Immune response gene and immune activation gene are used interchangeably herein.

An important technique for the analysis of the genes expressed by cells, such as cancer/tumour cells, is DNA microarray (also known as gene chip technology), where hundreds or more probe sequences (such as 55,000 probe sets) are attached to a glass surface. The probe sequences are generally all 25 mers or 60 mers and are sequences from known genes. These probes are generally arranged in a set of 11 individual probes (a probe set) and are fixed in a predefined pattern on the glass surface. Once exposed to an appropriate biological sample these probes hybridise to the relevant RNA or DNA of a particular gene. After washing, the chip is “read” by an appropriate method and a quantity such as colour intensity recorded. The differential expression of a particular gene is proportional to the measure/intensity recorded. This technology is discussed in more detail below.

Once a target gene profile has been identified there are several analytical methods to measure whether the gene(s) is/are differentially expressed. These analytical techniques include real-time polymerase chain reaction, also called quantitative real time polymerase chain reaction (QRT-PCR or Q-PCR), which is used to simultaneously quantify and amplify a specific part of a given DNA molecule present in the sample.

The procedure follows the general pattern of polymerase chain reaction, but the DNA is quantified after each round of amplification (the “real-time” aspect). Two common methods of quantification are the use of fluorescent dyes that intercalate with double-strand DNA, and modified DNA oligonucleotide probes that fluoresce when hybridized with a complementary DNA.

The basic idea behind real-time polymerase chain reaction is that the more abundant a particular cDNA (and thus mRNA) is in a sample, the earlier it will be detected during repeated cycles of amplification. Various systems exist which allow the amplification of DNA to be followed and they often involve the use of a fluorescent dye which is incorporated into newly synthesised DNA molecules during real-time amplification. Real-time polymerase chain reaction machines, which control the thermocycling process, can then detect the abundance of fluorescent DNA and thus the amplification progress of a given sample. Typically, amplification of a given cDNA over time follows a curve, with an initial flat-phase, followed by an exponential phase. Finally, as the experiment reagents are used up, DNA synthesis slows and the exponential curve flattens into a plateau.

Alternatively the mRNA or protein product of the target gene(s) may be measured by Northern Blot analysis, Western Blot and/or immunohistochemistry.

In one aspect the analysis to identify the profile/signature is performed on a patient sample wherein a cancer testis antigen is expressed.

If a gene is always upregulated or always down regulated in patients that are deemed to be responders (or alternatively non-responders) then this single gene can be used to establish if the patient is a responder or a non-responder once a threshold is established and provided the separation of the two groups is adequate.

When a single gene is analysed, for example, by Q-PCR then the gene expression can be normalised by reference to a gene that remains constant, for example genes with the symbol H3F3A, GΔPDH, TFRC, GUSB or PGK1. The normalisation can be performed by substracting the value obtained for the constant gene from the value obtained for the gene under consideration. A threshold may be established by plotting a measure of the expression of the relevant gene for each patient. Generally the responders and the non-responders will be clustered about a different axis/focal point. A threshold can be established in the gap between the clusters by classical statistical methods or simply plotting a “best fit line” to establish the middle ground between the two groups. Values, for example, above the pre-defined threshold can be designated as responders and values, for example below the pre-designated threshold can be designated as non-responders.

In one aspect the invention provides a gene profile for identifying a responder comprising one or more of said genes wherein 50, 60, 70, 75, 80, 85, 90, 95, 99 or 100% of the genes are upregulated.

The genes listed in Table 1, 2, 3, 4, 7, 9, 11, 12 and/or 13 such as Table 1, 2 and/or 3 are generally upregulated in responders.

The robustness of the predictive method of the invention can be further improved for larger sample sizes by employing 2, 3, 4, 5, 6, etc genes from Table 1, 2, 3, 4, 7, 9, 11, 12 and/or 13 such as Table 1 or 2.

Furthermore, once at least two differential expressed genes are included in the signature then statistical clustering methods can be used to differentiate the responders and non-responders. Methods for statistical clustering and software for the same are discussed below.

One parameter used in quantifying the differential expression of genes is the fold change, which is a metric for comparing a gene's mRNA-expression level between two distinct experimental conditions. Its arithmetic definition differs between investigators. However, the higher the fold change the more likely that the differential expression of the relevant genes will be adequately separated, rendering it easier to decide which category (responder or non-responder) the patient falls into.

The fold change may, for example be at least 10, at least 15, at least 20 or 30.

Another parameter also used to quantify differential expression is the “p” value. It is thought that the lower the p value the more differentially expressed the gene is likely to be, which renders it a good candidate for use in profiles of the invention. P values may for example include 0.1 or less, such as 0.05 or less, in particular 0.01 or less. P values as used herein include corrected “P” values and/or also uncorrected “P” values.

The invention provides a method for the detection of a gene signature in a biological sample, the method comprising the analysis of the expression of at least 5 genes as set forth in Table 1. Alternatively one or more genes may be selected from Table 1, 2, 3, 4, 7, 9, 11, 12 and/or 13 such as Table 1 or 2.

Thus in one aspect the invention provides a method of identifying whether a cancer patient will be a responder or non-responder to immunotherapy, the method comprising:
1. analysing a sample comprising mRNA or fragments thereof expressed by genes of cancerous cells or DNA or fragments thereof from cancerous cells, for differential expression of one or more genes indicative of T-cell infiltration/activation, for example selected from the group comprising or consisting of genes listed in Table 1, 2, 3, 4, 7, 9, 11, 12 and/or 13 such as Table 1 or 2, and
2. characterising a patient as a responder or a non-responder based on the results of step 1.

Responder in the context of the present invention includes persons where the cancer/tumor(s) is irradiated, reduced or improved (mixed responder or partial responder) or simply stabilised such that the disease is not progressing. In responders where the cancer is stabilised then the period of stabilisation is such that the quality of life and/or patients life expectancy is increased (for example stable disease for more than 6 months) in comparison to a patient that does not receive treatment.

Partial clinical response in respect of cancer is wherein all of the tumors/cancers respond to treatment to some extent, for example where said cancer is reduced by 30, 40, 50, 60% or more. Mixed clinical responder in respect of cancer is defined as wherein some of the tumors/cancers respond to treatment and others remain unchanged or progress.

Standard definitions are available for responders, partial responders and mixed responders to cancer treatment. These standard definitions apply herein unless from the context it is clear that they do not apply.

As used herein, methods to predict a favorable clinical response or to identify subjects more likely to respond to therapy, is not meant to imply a 100% predictive ability, but to indicate that subjects with certain characteristics are more likely to experience a favorable clinical response to a specified therapy than subjects who lack such characteristics. However, as will be apparent to one skilled in the art, some individuals identified as more likely to experience a favorable clinical response may nonetheless fail to demonstrate measurable clinical response to the treatment. Similarly, some individuals predicted as non-responders may nonetheless exhibit a favorable clinical response to the treatment.

As used herein, a ‘favorable response’ (or ‘favorable clinical response’) to, for example, an anticancer treatment refers to a biological or physical response that is recognized by those skilled in the art as indicating a decreased rate of tumor growth, compared to tumor growth that would occur with an alternate treatment or the absence of any treatment. “Favorable clinical response” as used herein is not synonymous with a cure, but includes Partial Response, Mixed Response or Stable Disease. A favorable clinical response to therapy may include a lessening of symptoms experienced by the subject, an increase in the expected or achieved survival time, a decreased rate of tumor growth, cessation of tumor growth (stable disease), regression in the number or mass of metastatic lesions, and/or regression of the overall tumor mass (each as compared to that which would occur in the absence of therapy, or in response to an alternate therapy).

Patients in need of treatment for, for example, a Mage-expressing tumor, whose tumor cells have a gene signature described herein as a “Responder” signature are more likely to have a favorable clinical response, compared to patients whose tumor cells show a gene signature described herein as a “Non-Responder” signature, when treated with Mage specific immunotherapy.

Non-responder in the context of this invention includes persons whose symptoms ie cancers/tumors are not improved or stabilised.

Optionally the characterisation of the patient as a responder or non-responder can be performed by reference to a “standard” or a training set. The standard may be the profile of a person/patient who is known to be a responder or non-responder or alternatively may be a numerical value. Such pre-determined standards may be provided in any suitable form, such as a printed list or diagram, computer software program, or other media.

Training set in the context of the present specification is intended to refer to a group of samples for which the clinical results can be correlated with the gene profile and can be employed for training an appropriate stastical model/programme to identify responders and/or non-responser for new samples. Tables 11A, 11B and 14 contain the training set information relevant to the 41 probe set model described in Example 4.

In one aspect a mathematical model/algorithm/statical method is employed to characterise the patient as responder or non-responder.

In one aspect the invention provides a profile based one or more immune activation genes, such as 5 or more such genes.

In one aspect the invention provides a profile based on the genes in Table 1, 2, 3, 4, 7, 9, 11, 12 and/or 13 such as Table 1 or 2.

In one aspect the invention provides a profile based on 489 probes (listed in Table 4A) and/or approximately 480 genes as listed in Table 4.

According to one aspect the present invention provides a gene signature indicative of improved survival of patients with Mage expressing cancers following treatment with Mage specific immunotherapy. This gene signature, of at least 1, 2, 3, 4, 5, 6, 7, 8, 9, 10, 11, 12, 13, 14, 15, 16, 17, 18, 19, 20 21, 22, 23, 24, 25, 26, 27, 28, 29, 30, or 31, genes from the genes disclosed in Table 1, is characterised by differential expression compared to the gene signature of MAGE-expressing tumour patients who do not respond to Mage antigen specific cancer immunotherapy. Improved survival in likely to be a corollary of a response to the immunotherapy administered, if the patient is in fact a responder.

Tables with Gene Lists

In one aspect the invention relates to one or more genes listed in Table 1

TABLE 1

| | Gene Title | Seq Id No |
|---|---|---|
| 1.1 | chromosome 6 open reading frame 190 | 1 |
| 1.2 | hematopoietic cell-specific Lyn substrate 1 | 2 |
| 1.3 | interleukin 2 receptor, gamma (severe combined | 3 |
| | immunodeficiency) | |
| 1.4 | CD52 antigen (CAMPATH-1 antigen) /// CD52 antigen | 4 |
| | (CAMPATH-1 antigen) | |
| 1.5 | CD2 antigen (p50), sheep red blood cell receptor /// CD2 antigen | 5 |
| | (p50), sheep red blood cell receptor | |
| 1.6 | ubiquitin D | 6 |
| 1.7 | signal transducer and activator of transcription 4 | 7 |
| 1.8 | granzyme K (granzyme 3; tryptase II)///granzyme K (granzyme 3; | 8 |
| | tryptase II) | |
| 1.9 | CD3G antigen, gamma polypeptide (TiT3 complex) | 9 |
| 1.10 | G protein-coupled receptor 171 | 10 |
| 1.11 | Protein kinase C, beta 1 | 11 |
| 1.12 | major histocompatibility complex, class II, DR alpha /// major | 12 |
| | histocompatibility complex, class II, DR alpha | |
| 1.13 | Major histocompatibility complex, class II, DQ beta 1 /// Major | 13 |
| | histocompatibility complex, class II, DQ beta 1 | |
| 1.14 | alcohol dehydrogenase IB (class I), beta polypeptide | 14 |
| 1.15 | T cell receptor alpha constant /// T cell receptor alpha constant | 15 |
| 1.16 | CD69 antigen (p60, early T-cell activation antigen) | 16 |
| 1.17 | protein kinase C, theta | 17 |
| 1.18 | T cell receptor beta variable 19 /// T cell receptor beta constant 1 | 18 |
| 1.19 | T cell receptor alpha locus /// T cell receptor delta variable 2 /// T | 19 |
| | cell receptor alpha variable 20 /// T cell receptor alpha joining 17 /// | |
| | T cell receptor alpha constant | |
| 1.20 | T cell receptor gamma constant 2 | 20 |
| 1.21 | T cell receptor beta variable 21-1 /// T cell receptor beta variable 19 | 21 |
| | /// T cell receptor beta variable 5-4 /// T cell receptor beta variable | |
| | 3-1 /// T cell receptor beta constant 1 | |
| 1.22 | T cell receptor alpha locus | 22 |
| 1.23 | T cell receptor beta variable 19 /// T cell receptor beta variable 19 /// | 23 |
| | T cell receptor beta constant 1 /// T cell receptor beta constant 1 | |
| 1.24 | CD3D antigen, delta polypeptide (TiT3 complex) | 24 |
| 1.25 | T cell receptor gamma constant 2 /// T cell receptor gamma variable | 25 |
| | 9 /// similar to T-cell receptor gamma chain C region PT-gamma-1/2 | |
| | /// similar to T-cell receptor gamma chain V region PT-gamma-1/2 | |
| | precursor /// TCR gamma alternate reading frame protein | |
| 1.26 | pyrin and HIN domain family, member 1 | 26 |
| 1.27 | T cell receptor associated transmembrane adaptor 1 | 27 |
| 1.28 | SLAM family member 7 | 28 |
| 1.29 | chromosome 4 open reading frame 7 | 29 |
| 1.30 | Major histocompatibility complex, class II, DQ alpha 1 | 30 |
| 1.31 | Transcribed locus | 31 |
| 1.32 | Amphiphysin (Stiff-Man syndrome with breast cancer 128 kDa | 32 |
| | autoantigen) | |
| 1.33 | dendritic cell-associated lectin-1 | 33 |

Table 1A at the end of this specification gives the full nucleotide sequence for each of the above genes.

In one aspect the invention provides a profile based on differential expression of 1 or more of 62 genes, identified in the literature, that relate to immune infiltration and activation.

In one aspect the invention provides a profile based on the genes listed in Table 2.

TABLE 2

| | Gene Symbol | Gene Title | Seq Id No |
|---|---|---|---|
| 2.1 | HLA-DQA1 | major histocompatibility complex, class II, DQ alpha 1 | 30 |
| 2.2 | TRBV3-1 | T cell receptor beta variable 3-1 | 21 |
| 2.3 | IL2RG | interleukin 2 receptor, gamma (severe combined | 3 |
| | | immunodeficiency | |
| 2.4 | CD2 | CD2 antigen (p50), sheep red blood cell receptor | 5 |
| 2.5 | GPR171 | G protein-coupled receptor 171 | 10 |
| 2.6 | ADH1B | alcohol dehydrogenase IB (class I), beta polypeptide | 14 |
| 2.7 | CD69 | CD69 antigen (p60, early T-cell activation antigen) | 16 |
| 2.8 | TRBV19 | T cell receptor beta variable 19 | 18 |
| 2.9 | TRAT1 | T cell receptor associated transmembrane adaptor 1 | 27 |
| 2.10 | C4orf7 | chromosome 4 open reading frame 7 | 29 |
| 2.11 | PRKCB1 | protein kinase C, beta 1 | 11 |
| 2.12 | CD3D | CD3D antigen, delta polypeptide (TiT3 complex) | 24 |
| 2.13 | UBD | ubiquitin D | 6 |

In one aspect the invention provides the list of genes in Table 3.

TABLE 3

| | Gene Symbol | Gene Title |
|---|---|---|
| 3.1 | CD52 | CD52 molecule /// CD52 molecule |
| 3.2 | UBD | gamma-aminobutyric acid (GABA) B receptor, 1 /// ubiquitin D |
| 3.3 | STAT4 | signal transducer and activator of transcription 4 |
| 3.4 | GZMK | granzyme K (granzyme 3; tryptase II) /// granzyme K (granzyme |
| | | 3; tryptase II) |
| 3.5 | GPR171 | G protein-coupled receptor 171 |
| 3.6 | PRKCQ | protein kinase C, theta |
| 3.7 | TRA@ /// TRDV2 /// | T cell receptor alpha locus /// T cell receptor delta variable 2 /// T |
| | TRAV20 /// TRAC | cell receptor alpha variable 20 /// T cell receptor alpha constant |
| 3.8 | TRGC2 /// TRGV2 /// | T cell receptor gamma constant 2 /// T cell receptor gamma |
| | TRGV9 /// TARP /// | variable 2 /// T cell receptor gamma variable 9 /// TCR gamma |
| | LOC642083 | alternate reading frame protein /// hypothetical protein |
| | | LOC642083 |
| 3.9 | TRBV21-1 /// | T cell receptor beta variable 21-1 /// T cell receptor beta variable |
| | TRBV19 /// TRBV5-4 | 19 /// T cell receptor beta variable 5-4 /// T cell receptor beta |
| | /// TRBV3-1 /// | variable 3-1 /// T cell receptor beta constant 1 /// similar to T-cell |
| | TRBC1 | receptor beta chain V region CTL-L17 precursor |
| 3.10 | TRBV19 /// TRBC1 | T cell receptor beta variable 19 /// T cell receptor beta variable 19 |
| | | /// T cell receptor beta constant 1 /// T cell receptor beta constant 1 |
| 3.11 | CD3D | CD3d molecule, delta (CD3-TCR complex) |
| 3.12 | TRAT1 | T cell receptor associated transmembrane adaptor 1 |

In one aspect the invention provides a profile based on one or more genes of Table 4

TABLE 4

| | Gene Symbol | Gene Title |
|---|---|---|
| 4.1 | FLJ20647 | NA |
| 4.2 | NAV1 | neuron navigator 1 |
| 4.3 | GPR171 | G protein-coupled receptor 171 |
| 4.4 | CCL14 | chemokine (C-C motif) ligand 14 |
| 4.5 | C1S | complement component 1, s subcomponent |
| 4.6 | CXCL2 | chemokine (C—X—C motif) ligand 2 |
| 4.7 | TRBV3-1 | T cell receptor beta variable 3-1 |
| 4.8 | TRDV2 | T cell receptor delta variable 2 |
| 4.9 | RUFY3 | RUN and FYVE domain containing 3 |
| 4.10 | DOCK8 | dedicator of cytokinesis 8 |
| 4.11 | GCH1 | GTP cyclohydrolase 1 (dopa-responsive dystonia) |
| 4.12 | CENTD3 | centaurin, delta 3 |
| 4.13 | ACSL5 | acyl-CoA synthetase long-chain family member 5 |
| 4.14 | AMICA1 | adhesion molecule, interacts with CXADR antigen 1 |
| 4.15 | IL2RG | interleukin 2 receptor, gamma (severe combined |
| | | immunodeficiency) |
| 4.16 | TNFAIP3 | tumor necrosis factor, alpha-induced protein 3 |
| 4.17 | PSCDBP | pleckstrin homology, Sec7 and coiled-coil domains, binding |
| | | protein |
| 4.18 | ESR1 | estrogen receptor 1 |
| 4.19 | TRBC1 | T cell receptor beta constant 1 |
| 4.20 | CD52 | CD52 antigen (CAMPATH-1 antigen) |
| 4.21 | LOC442535 | NA |
| 4.22 | TRBV19 | T cell receptor beta variable 19 |
| 4.23 | IL7R | interleukin 7 receptor |
| 4.24 | TRAC | T cell receptor alpha constant |
| 4.25 | NCF2 | neutrophil cytosolic factor 2 (65 kDa, chronic granulomatous |
| | | disease, autosomal 2) |
| 4.26 | LOC92689 | NA |
| 4.27 | GZMK | c(“granzyme K (granzyme 3”, “tryptase II)”) |
| 4.28 | NA | NA (gene identified by probe 235831_at) |
| 4.29 | RAB34 | RAB34, member RAS oncogene family |
| 4.30 | DPT | dermatopontin |
| 4.31 | PVT1 | Pvt1 oncogene homolog, MYC activator (mouse) |
| 4.32 | TRGC2 | T cell receptor gamma constant 2 |
| 4.33 | GIMAP5 | GTPase, IMAP family member 5 |
| 4.34 | CD52 | CD52 antigen (CAMPATH-1 antigen) |
| 4.35 | CD3D | CD3d antigen, delta polypeptide (TiT3 complex) |
| 4.36 | TMEM132C | transmembrane protein 132C |
| 4.37 | NFKBIA | nuclear factor of kappa light polypeptide gene enhancer in B- |
| | | cells inhibitor, alpha |
| 4.38 | TRA@ | T cell receptor alpha locus |
| 4.39 | TRAT1 | T cell receptor associated transmembrane adaptor 1 |
| 4.40 | LOC442535 (NA) | NA (duplicate) |
| 4.41 | RAB34 | RAB34, member RAS oncogene family |
| 4.42 | CD69 | CD69 antigen (p60, early T-cell activation antigen) |
| 4.43 | DOCK8 | dedicator of cytokinesis 8 |
| 4.44 | GIMAP5 (NA) | GTPase, IMAP family member 5 (NA-duplicate) |
| 4.45 | IRF8 | interferon regulatory factor 8 |
| 4.46 | KLRB1 | killer cell lectin-like receptor subfamily B, member 1 |
| 4.47 | NA | NA |
| 4.48 | ITGA3 | integrin, alpha 3 (antigen CD49C, alpha 3 subunit of VLA-3 |
| | | receptor) |
| 4.49 | MAP3K8 | mitogen-activated protein kinase kinase kinase 8 |
| 4.50 | C1orf162 | chromosome 1 open reading frame 162 |
| 4.51 | UBD | ubiquitin D |
| 4.52 | TRGV9 | T cell receptor gamma variable 9 |
| 4.53 | NAV1 (NA) | neuron navigator 1 (NA-duplicate) |
| 4.54 | ARHGAP9 | Rho GTPase activating protein 9 |
| 4.55 | TIFA | NA |
| 4.56 | DPT | dermatopontin |
| 4.57 | NA | NA (gene identified by probe 1569942_at) |
| 4.58 | GIMAP4 | GTPase, IMAP family member 4 |
| 4.59 | HCLS1 | hematopoietic cell-specific Lyn substrate 1 |
| 4.60 | PRKCH | protein kinase C, eta |
| 4.61 | STAT4 | signal transducer and activator of transcription 4 |
| 4.62 | HLA-DQA1 | major histocompatibility complex, class II, DQ alpha 1 |
| 4.63 | ADRB2 | adrenergic, beta-2-, receptor, surface |
| 4.64 | NA | NA |
| 4.65 | CTSW | cathepsin W (lymphopain) |
| 4.66 | MYH11 | myosin, heavy polypeptide 11, smooth muscle |
| 4.67 | GIMAP6 | GTPase, IMAP family member 6 |
| 4.68 | HLA-DQB1 | major histocompatibility complex, class II, DQ beta 1 |
| 4.69 | CD8A | CD8 antigen, alpha polypeptide (p32) |
| 4.70 | TNFAIP3 | tumor necrosis factor, alpha-induced protein 3 |
| 4.71 | CP | ceruloplasmin (ferroxidase) |
| 4.72 | SMOC2 | SPARC related modular calcium binding 2 |
| 4.73 | C20orf24 | chromosome 20 open reading frame 24 |
| 4.74 | C16orf54 | chromosome 16 open reading frame 54 |
| 4.75 | CD2 | CD2 antigen (p50), sheep red blood cell receptor |
| 4.76 | SLIT3 | slit homolog 3 (Drosophila) |
| 4.77 | BAALC | brain and acute leukemia, cytoplasmic |
| 4.78 | TRIB3 | tribbles homolog 3 (Drosophila) |
| 4.79 | LOC440160 | NA |
| 4.80 | C6orf190 | chromosome 6 open reading frame 190 |
| 4.81 | TAGAP | T-cell activation GTPase activating protein |
| 4.82 | FAM92A1 | family with sequence similarity 92, member A1 |
| 4.83 | PSTPIP2 | proline-serine-threonine phosphatase interacting protein 2 |
| 4.84 | PTPRC | protein tyrosine phosphatase, receptor type, C |
| 4.85 | HLA-DRA | major histocompatibility complex, class II, DR alpha |
| 4.86 | EFCAB2 | EF-hand calcium binding domain 2 |
| 4.87 | TNFAIP8 | tumor necrosis factor, alpha-induced protein 8 |
| 4.88 | SLIC1 | NA |
| 4.89 | CD1C | CD1c antigen |
| 4.90 | TRAF3IP3 | TRAF3 interacting protein 3 |
| 4.91 | HLA-DQA1 (NA) | MHC, class II, DQ α-1 (NA-duplicate) |
| 4.92 | NAV1 (NA) | neuron navigator 1 (NA-duplicate) |
| 4.93 | HLA-DQB1 (NA) | MHC, class II, DQ beta 1 (NA-duplicate) |
| 4.94 | PTPRC (NA) | protein tyrosine phosphatase, receptor type, C (NA-duplicate) |
| 4.95 | IGJ | immunoglobulin J polypeptide, linker protein for |
| | | immunoglobulin alpha and mu polypeptides |
| 4.96 | PLEK | pleckstrin |
| 4.97 | TRA@ (NA) | T cell receptor alpha locus (NA-duplicate) |
| 4.98 | TMEM44 | transmembrane protein 44 |
| 4.99 | TRA@ | T cell receptor alpha locus |
| 4.100 | EBI2 | Epstein-Barr virus induced gene 2 (lymphocyte-specific G |
| | | protein-coupled receptor) |
| 4.101 | SAMSN1 | SAM domain, SH3 domain and nuclear localisation signals, 1 |
| 4.102 | KIAA1794 | KIAA1794 |
| 4.103 | ALDH2 | aldehyde dehydrogenase 2 family (mitochondrial) |
| 4.104 | CDC42SE2 | CDC42 small effector 2 |
| 4.105 | GFRA1 | GDNF family receptor alpha 1 |
| 4.106 | ITK | IL2-inducible T-cell kinase |
| 4.107 | HLA-DRA | major histocompatibility complex, class II, DR alpha |
| 4.108 | GIMAP7 | GTPase, IMAP family member 7 |
| 4.109 | FLJ20273 | NA |
| 4.110 | PTPN6 | protein tyrosine phosphatase, non-receptor type 6 |
| 4.111 | PTGER3 | prostaglandin E receptor 3 (subtype EP3) |
| 4.112 | RAI2 | retinoic acid induced 2 |
| 4.113 | LGALS2 | lectin, galactoside-binding, soluble, 2 (galectin 2) |
| 4.114 | HMOX1 | heme oxygenase (decycling) 1 |
| 4.115 | NA | NA (gene identified by probe 227995_at) |
| 4.116 | ZNFN1A1 | zinc finger protein, subfamily 1A, 1 (Ikaros) |
| 4.117 | CSF2RB | colony stimulating factor 2 receptor, beta, low-affinity |
| | | (granulocyte-macrophage) |
| 4.118 | PCSK5 | proprotein convertase subtilisin/kexin type 5 |
| 4.119 | CCDC69 | coiled-coil domain containing 69 |
| 4.120 | CDC42SE2 | CDC42 small effector 2 |
| 4.121 | GZMA | granzyme A (granzyme 1, cytotoxic T-lymphocyte-associated |
| | | serine esterase 3) |
| 4.122 | C3 | complement component 3 |
| 4.123 | TNFAIP8 (NA) | tumor necrosis factor, alpha-induced protein 8 (NA-duplicate) |
| 4.124 | C15orf48 | chromosome 15 open reading frame 48 |
| 4.125 | RARRES3 | retinoic acid receptor responder (tazarotene induced) 3 |
| 4.126 | LOC283537 | NA |
| 4.127 | CXCL12 | chemokine (C—X—C motif) ligand 12 (stromal cell-derived factor |
| | | 1) |
| 4.128 | NAV1 (NA) | neuron navigator 1 (NA-duplicate) |
| 4.129 | NA | NA (gene identified by probe set 231882_at) |
| 4.130 | SOD2 | superoxide dismutase 2, mitochondrial |
| 4.131 | CTSS | cathepsin S |
| 4.132 | CTBP2 | C-terminal binding protein 2 |
| 4.133 | BCL11B | B-cell CLL/lymphoma 11B (zinc finger protein) |
| 4.134 | CCL22 | chemokine (C-C motif) ligand 22 |
| 4.135 | ACSL5 | acyl-CoA synthetase long-chain family member 5 |
| 4.136 | DOC1 | NA |
| 4.137 | SLC31A2 | solute carrier family 31 (copper transporters), member 2 |
| 4.138 | POPDC3 | popeye domain containing 3 |
| 4.139 | DOC1 (NA) | NA (duplicate) |
| 4.140 | SQRDL | sulfide quinone reductase-like (yeast) |
| 4.141 | RASGEF1B | RasGEF domain family, member 1B |
| 4.142 | FGL2 | fibrinogen-like 2 |
| 4.143 | C10orf128 | chromosome 10 open reading frame 128 |
| 4.144 | IL10RA | interleukin 10 receptor, alpha |
| 4.145 | EGFL6 | EGF-like-domain, multiple 6 |
| 4.146 | IL18 | interleukin 18 (interferon-gamma-inducing factor) |
| 4.147 | ARHGAP30 | Rho GTPase activating protein 30 |
| 4.148 | PALMD | palmdelphin |
| 4.149 | RASSF5 | Ras association (RalGDS/AF-6) domain family 5 |
| 4.150 | GATA3 | GATA binding protein 3 |
| 4.151 | DKFZP564O0823 | NA |
| 4.152 | BCL11B | B-cell CLL/lymphoma 11B (zinc finger protein) |
| 4.153 | TXNIP | thioredoxin interacting protein |
| 4.154 | DTX4 | deltex 4 homolog (Drosophila) |
| 4.155 | DARC | Duffy blood group, chemokine receptor |
| 4.156 | RNASE6 | ribonuclease, RNase A family, k6 |
| 4.157 | CD86 | CD86 antigen (CD28 antigen ligand 2, B7-2 antigen) |
| 4.158 | ZFP36 | zinc finger protein 36, C3H type, homolog (mouse) |
| 4.159 | BASP1 | brain abundant, membrane attached signal protein 1 |
| 4.160 | CKAP1 | cytoskeleton associated protein 1 |
| 4.161 | HCP5 | HLA complex P5 |
| 4.162 | GRB14 | growth factor receptor-bound protein 14 |
| 4.163 | GJA7 | gap junction protein, alpha 7, 45 kDa (connexin 45) |
| 4.164 | FLJ14054 | NA |
| 4.165 | VNN1 | vanin 1 |
| 4.166 | ADCY7 | adenylate cyclase 7 |
| 4.167 | MS4A6A | membrane-spanning 4-domains, subfamily A, member 6A |
| 4.168 | CPA3 | carboxypeptidase A3 (mast cell) |
| 4.169 | PIM1 | pim-1 oncogene |
| 4.170 | CCL19 | chemokine (C-C motif) ligand 19 |
| 4.171 | SYK | spleen tyrosine kinase |
| 4.172 | NAV1 (NA) | neuron navigator 1 (NA-duplicate) |
| 4.173 | SIT1 | signaling threshold regulating transmembrane adaptor 1 |
| 4.174 | NA | NA (gene identified by probe set 228812_at) |
| 4.175 | NAP1L2 | nucleosome assembly protein 1-like 2 |
| 4.176 | CCL13 | chemokine (C-C motif) ligand 13 |
| 4.177 | SLA | Src-like-adaptor |
| 4.178 | NOD3 | NA |
| 4.179 | PRKCH | protein kinase C, eta |
| 4.180 | TRD@ | T cell receptor delta locus |
| 4.181 | BAALC | brain and acute leukemia, cytoplasmic |
| 4.182 | RP1-93H18.5 | NA |
| 4.183 | FLJ20701 | NA |
| 4.184 | SH3TC2 | SH3 domain and tetratricopeptide repeats 2 |
| 4.185 | CCR2 | chemokine (C-C motif) receptor 2 |
| 4.186 | CCL5 | chemokine (C-C motif) ligand 5 |
| 4.187 | HLA-DPA1 | major histocompatibility complex, class II, DP alpha 1 |
| 4.188 | MS4A6A (NA) | membrane spanning 4-domains, subfamilyA, member6A (NA- |
| | | duplicate) |
| 4.189 | PECAM1 | platelet/endothelial cell adhesion molecule (CD31 antigen) |
| 4.190 | AMIGO2 | adhesion molecule with Ig-like domain 2 |
| 4.191 | CCDC69 | coiled-coil domain containing 69 |
| 4.192 | CLEC7A | C-type lectin domain family 7, member A |
| 4.193 | P2RY14 | purinergic receptor P2Y, G-protein coupled, 14 |
| 4.194 | PIK3AP1 | phosphoinositide-3-kinase adaptor protein 1 |
| 4.195 | ADH1B | alcohol dehydrogenase IB (class I), beta polypeptide |
| 4.196 | TOP1MT | topoisomerase (DNA) I, mitochondrial |
| 4.197 | CD276 | CD276 antigen |
| 4.198 | HLA-DQB1 | major histocompatibility complex, class II, DQ beta I |
| 4.199 | JAM2 | junctional adhesion molecule 2 |
| 4.200 | C1S | complement component 1, s subeomponent |
| 4.201 | MS4A6A | membrane-spanning 4-domains, subfamily A, member 6A |
| 4.202 | TGFBR3 | transforming growth factor, beta receptor III (betaglycan, 300 kDa) |
| 4.203 | ITGAL | c(“integrin, alpha L (antigen CD11A (p180), lymphocyte function- |
| | | associated antigen 1”, “alpha polypeptide)”) |
| 4.204 | IL1R1 | interleukin 1 receptor, type I |
| 4.205 | MS4A6A | membrane-spanning 4-domains, subfamily A, member 6A |
| 4.206 | HLA-DRB1 | major histocompatibility complex, class II, DR beta 1 |
| 4.207 | GIMAP2 | GTPase, IMAP family member 2 |
| 4.208 | ZC3H12D | zinc finger CCCH-type containing 12D |
| 4.209 | PCDH9 | protocadherin 9 |
| 4.210 | SLAMF7 | SLAM family member 7 |
| 4.211 | MGC7036 | NA |
| 4.212 | RGS18 | regulator of G-protein signalling 18 |
| 4.213 | HLA-DQA1 | major histocompatibility complex, class II, DQ alpha 1 |
| 4.214 | CD53 | CD53 antigen |
| 4.215 | MPEG1 | NA |
| 4.216 | SSBP4 | single stranded DNA binding protein 4 |
| 4.217 | NA | NA (gene identified by probe set 231262_at) |
| 4.218 | CDH19 | cadherin 19, type 2 |
| 4.219 | CTBP2 | C-terminal binding protein 2 |
| 4.220 | NAV1 (NA) | neuron navigator 1 (NA-duplicate) |
| 4.221 | FAM107B | family with sequence similarity 107, member B |
| 4.222 | IGKC | immunoglobulin kappa constant |
| 4.223 | ITGAM | integrin, alpha M (complement component 3 receptor 3 subunit) |
| 4.224 | CKAP1 | cytoskeleton associated protein 1 |
| 4.225 | HLA-DRB1 | major histocompatibility complex, class II, DR beta 1 |
| 4.226 | CDH19 | cadherin 19, type 2 |
| 4.227 | MGC16291 | NA |
| 4.228 | DDEF2 | development and differentiation enhancing factor 2 |
| 4.229 | TNFAIP2 | tumor necrosis factor, alpha-induced protein 2 |
| 4.230 | CXCL14 | chemokine (C—X—C motif) ligand 14 |
| 4.231 | CD209 | CD209 antigen |
| 4.232 | COL9A3 | collagen, type IX, alpha 3 |
| 4.233 | ANKRD22 | ankyrin repeat domain 22 |
| 4.234 | NCKAP1L | NCK-associated protein 1-like |
| 4.235 | CMKOR1 | chemokine orphan receptor 1 |
| 4.236 | HLA-DRB5 | major histocompatibility complex, class II, DR beta 5 |
| 4.237 | LCP1 | lymphocyte cytosolic protein 1 (L-plastin) |
| 4.238 | ADH1B | alcohol dehydrogenase IB (class I), beta polypeptide |
| 4.239 | CXXC5 | CXXC finger 5 |
| 4.240 | GJA7 | gap junction protein, alpha 7, 45 kDa (connexin 45) |
| 4.241 | FGD2 | FYVE, RhoGEF and PH domain containing 2 |
| 4.242 | MAN1A1 | mannosidase, alpha, class 1A, member 1 |
| 4.243 | C6orf115 | chromosome 6 open reading frame 115 |
| 4.244 | RP1-93H18.5 (NA) | NA (duplicate) |
| 4.245 | CXCL9 | chemokine (C—X—C motif) ligand 9 |
| 4.246 | FAM107B (NA) | family with sequence similarity 107, member B (NA-duplicate) |
| 4.247 | NPR3 | natriuretic peptide receptor C/guanylate cyclase C (atrionatriuretic |
| | | peptide receptor C) |
| 4.248 | FYB | FYN binding protein (FYB-120/130) |
| 4.249 | VCAM1 | vascular cell adhesion molecule 1 |
| 4.250 | FLI1 | Friend leukemia virus integration 1 |
| 4.251 | CXXC5 | CXXC finger 5 |
| 4.252 | TRAM2 | translocation associated membrane protein 2 |
| 4.253 | IGKC (NA) | immunoglobulin kappa constant (NA-duplicate) |
| 4.254 | SHC4 | SHC (Src homology 2 domain containing) family, member 4 |
| 4.255 | SLC9A9 | solute carrier family 9 (sodium/hydrogen exchanger), member 9 |
| 4.256 | PTPRC | protein tyrosine phosphatase, receptor type, C |
| 4.257 | PTGER4 | prostaglandin E receptor 4 (subtype EP4) |
| 4.258 | LILRB1 | leukocyte immunoglobulin-like receptor, subfamily B (with TM |
| | | and ITIM domains), member 1 |
| 4.259 | PRDM1 | PR domain containing 1, with ZNF domain |
| 4.260 | RP1-93H18.5 (NA) | NA (duplicate) |
| 4.261 | ARHGAP15 | Rho GTPase activating protein 15 |
| 4.262 | SLC5A3 | solute carrier family 5 (inositol transporters), member 3 |
| 4.263 | DOCK9 | dedicator of cytokinesis 9 |
| 4.264 | GPSM1 | G-protein signalling modulator 1 (AGS3-like, C. elegans) |
| 4.265 | CCL5 | chemokine (C-C motif) ligand 5 |
| 4.266 | GLIPR1 | GLI pathogenesis-related 1 (glioma) |
| 4.267 | APOL3 | apolipoprotein L, 3 |
| 4.268 | HLA-DMB | major histocompatibility complex, class II, DM beta |
| 4.269 | SYNPO2 | synaptopodin 2 |
| 4.270 | NA | NA (gene identified by probe set 221651_x_at) |
| 4.271 | NA | NA (gene identified by probe set 231929_at) |
| 4.272 | RP1-93H18.5 | NA |
| 4.273 | CASP1 | caspase 1, apoptosis-related cysteine peptidase (interleukin 1, beta, |
| | | convertase) |
| 4.274 | PRKCQ | protein kinase C, theta |
| 4.275 | IL1R2 | interleukin 1 receptor, type II |
| 4.276 | CARD15 | caspase recruitment domain family, member 15 |
| 4.277 | ARHGDIB | Rho GDP dissociation inhibitor (GDI) beta |
| 4.278 | HLA-DRB4 | major histocompatibility complex, class II, DR beta 4 |
| 4.279 | SART2 | squamous cell carcinoma antigen recognized by T cells 2 |
| 4.280 | LSP1 | lymphocyte-specific protein 1 |
| 4.281 | AMPD3 | adenosine monophosphate deaminase (isoform E) |
| 4.282 | SEMA4F | sema domain, immunoglobulin domain (Ig), transmembrane |
| | | domain (TM) and short cytoplasmic domain, (semaphorin) 4F |
| 4.283 | ISOC1 | isochorismatase domain containing 1 |
| 4.284 | CCL5 (NA) | chemokine (C-C motif) ligand 5 (NA-duplicate) |
| 4.285 | HPS3 | Hermansky-Pudlak syndrome 3 |
| 4.286 | HLA-DPA1 (NA) | MHC, class II, DP alpha 1 (NA-duplicate) |
| 4.287 | HLA-DQB1 (NA) | MHC, class II, DQ beta 1 (NA-duplicate) |
| 4.288 | HOXB7 | homeobox B7 |
| 4.289 | FGL2 (NA) | fibrinogen-like 2 (NA-duplicate) |
| 4.290 | ZNFN1A1 | zinc finger protein, subfamily 1A, 1 (Ikaros) |
| 4.291 | ARHGAP9 | Rho GTPase activating protein 9 |
| 4.292 | GATA2 | GATA binding protein 2 |
| 4.293 | AP2B1 | adaptor-related protein complex 2, beta 1 subunit |
| 4.294 | CTSC | cathepsin C |
| 4.295 | PLK2 | polo-like kinase 2 (Drosophila) |
| 4.296 | CD4 | CD4 antigen (p55) |
| 4.297 | GGTA1 | glycoprotein, alpha-galactosyltransferase 1 |
| 4.298 | GADD45B | growth arrest and DNA-damage-inducible, beta |
| 4.299 | GADD45B (NA) | growth arrest and DNA-damage-inducible, beta (NA-duplicate) |
| 4.300 | FLJ10847 | NA |
| 4.301 | KIF21B | kinesin family member 21B |
| 4.302 | CCND2 | cyclin D2 |
| 4.303 | PRG1 | proteoglycan 1, secretory granule |
| 4.304 | SLC40A1 | solute carrier family 40 (iron-regulated transporter), member 1 |
| 4.305 | HLA-DPA1 (NA) | MHC, class II, DP alpha 1 (NA-duplicate) |
| 4.306 | SOD2 (NA) | superoxide dismutase 2, mitochondrial (NA-duplicate) |
| 4.307 | CRIP1 | cysteine-rich protein 1 (intestinal) |
| 4.308 | LOC283070 | NA |
| 4.309 | SIGLEC1 | sialic acid binding Ig-like lectin 1, sialoadhesin |
| 4.310 | ZNF11B | zinc finger protein 11B |
| 4.311 | CXCR4 | chemokine (C—X—C motif) receptor 4 |
| 4.312 | HLA-DMA | major histocompatibility complex, class II, DM alpha |
| 4.313 | MRC1 | mannose receptor, C type 1 |
| 4.314 | CASP1 (NA) | caspase 1, apoptosis-related cysteine peptidase (interleukin 1, beta, |
| | | convertase) (NA-duplicate) |
| 4.315 | LMO2 | LIM domain only 2 (rhombotin-like 1) |
| 4.315a | DENND2D | DENN/MADD domain containing 2D |
| 4.316 | CCL18 | chemokine (C-C motif) ligand 18 (pulmonary and activation- |
| | | regulated) |
| 4.317 | P2RY13 | purinergic receptor P2Y, G-protein coupled, 13 |
| 4.318 | CCL18 (NA) | chemokine (C-C motif) ligand 18 (pulmonary and activation- |
| | | regulated) (NA-duplicate) |
| 4.319 | ANGPTL1 | angiopoietin-like 1 |
| 4.320 | NA | NA (gene identified by probe set 230391_at) |
| 4.321 | ITGAL (NA) | c(“integrin, alpha L (antigen CD11A (p180), lymphocyte function- |
| | | associated antigen 1”, “alpha polypeptide)”) (NA-duplicate) |
| 4.322 | C8orf51 | chromosome 8 open reading frame 51 |
| 4.323 | GIMAP8 | GTPase, IMAP family member 8 |
| 4.324 | NA | NA (gene identified by probe set 227780_s_at) |
| 4.325 | JAK2 | Janus kinase 2 (a protein tyrosine kinase) |
| 4.326 | TNFSF10 | tumor necrosis factor (ligand) superfamily, member 10 |
| 4.327 | C1R | complement component 1, r subcomponent |
| 4.328 | ACPL2 | acid phosphatase-like 2 |
| 4.329 | TNFRSF19 | tumor necrosis factor receptor superfamily, member 19 |
| 4.330 | PCSK5 (NA) | proprotein convertase subtilisin/kexin type 5 (NA-duplicate) |
| 4.331 | LRP12 | low density lipoprotein-related protein 12 |
| 4.332 | NA | NA (gene identified by probe set 1557116_at) |
| 4.333 | PECAM1 (NA) | platelet/endothelial cam (CD31 antigen) (NA-duplicate) |
| 4.334 | PRKCB1 | protein kinase C, beta 1 |
| 4.335 | IPO11 | importin 11 |
| 4.336 | DLGAP1 | discs, large (Drosophila) homolog-associated protein 1 |
| 4.337 | PRKAR2B | protein kinase, cAMP-dependent, regulatory, type II, beta |
| 4.338 | MAP3K8 | mitogen-activated protein kinase kinase kinase 8 |
| 4.339 | EVI2B | ecotropic viral integration site 2B |
| 4.340 | GBP1 | guanylate binding protein 1, interferon-inducible, 67 kDa |
| 4.341 | CXCL10 | chemokine (C—X—C motif) ligand 10 |
| 4.342 | CAMK2N1 | calcium/calmodulin-dependent protein kinase II inhibitor 1 |
| 4.343 | MED12L | mediator of RNA polymerase II transcription, subunit 12 homolog |
| | | (yeast)-like |
| 4.344 | ID2 | inhibitor of DNA binding 2, dominant negative helix-loop-helix |
| | | protein |
| 4.345 | CTBP2 | C-terminal binding protein 2 |
| 4.346 | IGLJ3 | immunoglobulin lambda joining 3 |
| 4.347 | GBP4 | guanylate binding protein 4 |
| 4.348 | LOC439949 | NA |
| 4.349 | FBXO16 | F-box protein 16 |
| 4.350 | PRF1 | perforin 1 (pore forming protein) |
| 4.351 | TRAM2 | translocation associated membrane protein 2 |
| 4.352 | LYN | v-yes-1 Yamaguchi sarcoma viral related oncogene homolog |
| 4.353 | CENTD1 | centaurin, delta 1 |
| 4.354 | CASP1 (NA) | caspase 1, apoptosis-related cysteine peptidase (interleukin 1, beta, |
| | | convertase) (NA-duplicate) |
| 4.355 | FLJ20273 | NA |
| 4.356 | TFEC | transcription factor EC |
| 4.357 | PPP1R16B | protein phosphatase 1, regulatory (inhibitor) subunit 16B |
| 4.358 | CD48 | CD48 antigen (B-cell membrane protein) |
| 4.359 | HLA-DPB1 | major histocompatibility complex, class II, DP beta 1 |
| 4.360 | SHC4 (NA) | SHC family, member 4 (NA-duplicate) |
| 4.361 | GTPBP5 | GTP binding protein 5 (putative) |
| 4.362 | GBP5 | guanylate binding protein 5 |
| 4.363 | MAP1B | microtubule-associated protein 1B |
| 4.364 | EXTL3 | exostoses (multiple)-like 3 |
| 4.365 | CORO1A | coronin, actin binding protein, 1A |
| 4.366 | PDGFRL | platelet-derived growth factor receptor-like |
| 4.367 | RP9 | retinitis pigmentosa 9 (autosomal dominant) |
| 4.368 | RHOU | ras homolog gene family, member U |
| 4.369 | MTAC2D1 | membrane targeting (tandem) C2 domain containing 1 |
| 4.370 | CCL8 | chemokine (C-C motif) ligand 8 |
| 4.371 | CECR1 | cat eye syndrome chromosome region, candidate 1 |
| 4.372 | IGKC (NA) | immunoglobulin kappa constant (NA-duplicate) |
| 4.373 | SLC40A1 | solute carrier family 40 (iron-regulated transporter), member 1 |
| 4.374 | ADCY6 | adenylate cyclase 6 |
| 4.375 | CP | ceruloplasmin (ferroxidase) |
| 4.376 | EDG1 | endothelial differentiation, sphingolipid G-protein-coupled |
| | | receptor, 1 |
| 4.377 | RGS3 | regulator of G-protein signalling 3 |
| 4.378 | CD28 (NA) | CD28 antigen (Tp44) (NA-duplicate) |
| 4.379 | NA | NA (gene identified by probe set 228339_at) |
| 4.380 | ABHD5 | abhydrolase domain containing 5 |
| 4.381 | MS4A7 | membrane-spanning 4-domains, subfamily A, member 7 |
| 4.382 | PRKCH | protein kinase C, eta |
| 4.383 | GBP1 (NA) | guanylate binding protein 1, i-inducible, 67 kDa (NA-duplicate) |
| 4.384 | LOC286071 | NA |
| 4.385 | BLNK | B-cell linker |
| 4.386 | NA | NA (gene identified by probe set 242546_at) |
| 4.387 | PCDHGC3 | protocadherin gamma subfamily C, 3 |
| 4.388 | CCL13 (NA) | chemokine (C-C motif) ligand 13 (NA-duplicate) |
| 4.389 | JAK2 (NA) | Janus kinase 2 (a protein tyrosine kinase) (NA-duplicate) |
| 4.390 | CAMSAP1L1 | calmodulin regulated spectrin-associated protein 1-like 1 |
| 4.391 | NPY1R | neuropeptide Y receptor Y1 |
| 4.392 | CD274 | CD274 antigen |
| 4.393 | PGM5 | phosphoglucomutase 5 |
| 4.394 | PLCG2 | phospholipase C, gamma 2 (phosphatidylinositol-specific) |
| 4.395 | TNFSF10 | tumor necrosis factor (ligand) superfamily, member 10 |
| 4.396 | SOD2 (NA) | superoxide dismutase 2, mitochondrial (NA-duplicate) |
| 4.397 | BTG2 | BTG family, member 2 |
| 4.398 | LAMP3 | lysosomal-associated membrane protein 3 |
| 4.399 | IGLC1 | immunoglobulin lambda constant 1 (Mcg marker) |
| 4.400 | SIPA1L1 | signal-induced proliferation-associated 1 like 1 |
| 4.401 | AIF1 | allograft inflammatory factor 1 |
| 4.402 | IGLC2 | immunoglobulin lambda constant 2 (Kern-Oz-marker) |
| 4.403 | B2M | beta-2-microglobulin |
| 4.404 | CLEC7A | C-type lectin domain family 7, member A |
| 4.405 | MGC17330 | NA |
| 4.406 | IGF1R | insulin-like growth factor 1 receptor |
| 4.407 | HIVEP1 | human immunodeficiency virus type I enhancer binding protein 1 |
| 4.408 | FKBP14 | FK506 binding protein 14, 22 kDa |
| 4.409 | LAPTM5 | lysosomal associated multispanning membrane protein 5 |
| 4.410 | ABI3BP | ABI gene family, member 3 (NESH) binding protein |
| 4.411 | HLA-E | major histocompatibility complex, class I, E |
| 4.412 | ARL4C | ADP-ribosylation factor-like 4C |
| 4.413 | ASS | argininosuccinate synthetase |
| 4.414 | CASP1 (NA) | caspase 1, apoptosis-related cysteine peptidase (interleukin 1, beta, |
| | | convertase) (NA-duplicate) |
| 4.415 | ITGB3 | integrin, beta 3 (platelet glycoprotein IIIa, antigen CD61) |
| 4.416 | SYK | spleen tyrosine kinase |
| 4.417 | RAC2 | ras-related C3 botulinum toxin substrate 2 (rho family, small GTP |
| | | binding protein Rac2) |
| 4.418 | NA | NA (gene identified by probe set 1557222_at) |
| 4.419 | CD3G | CD3g antigen, gamma polypeptide (TiT3 complex) |
| 4.420 | IGF1 | insulin-like growth factor 1 (somatomedin C) |
| 4.421 | NA | NA (gene identified by probe set 228858_at) |
| 4.422 | CYB5A | cytochrome b5 type A (microsomal) |
| 4.423 | TTC25 | tetratricopeptide repeat domain 25 |
| 4.424 | SLAMF6 | SLAM family member 6 |
| 4.425 | ARHGAP21 | Rho GTPase activating protein 21 |
| 4.426 | FLOT1 | flotillin 1 |
| 4.427 | AIF1 (NA) | allograft inflammatory factor 1 (NA-duplicate) |
| 4.428 | IBRDC2 | IBR domain containing 2 |
| 4.429 | KIAA1794 | KIAA1794 |
| 4.430 | OLFML1 | olfactomedin-like 1 |
| 4.431 | GMFG | glia maturation factor, gamma |
| 4.432 | TNFRSF1B | tumor necrosis factor receptor superfamily, member 1B |
| 4.433 | NA | NA (gene identified by probe set 217629_at) |
| 4.434 | DEF6 | differentially expressed in FDCP 6 homolog (mouse) |
| 4.435 | HLA-E (NA) | major histocompatibility complex, class I, E (NA-duplicate) |
| 4.436 | MAP4K4 | mitogen-activated protein kinase kinase kinase kinase 4 |
| 4.437 | CMKOR1 | chemokine orphan receptor 1 |
| 4.438 | NA | NA (gene identified by probe set 1563461_at) |
| 4.439 | CHKA | choline kinase alpha |
| 4.440 | NA | NA (gene identified by probe set 226865_at) |
| 4.441 | HS3ST3B1 | heparan sulfate (glucosamine) 3-O-sulfotransferase 3B1 |
| 4.442 | CXorf9 | chromosome X open reading frame 9 |
| 4.443 | EVI2A | ecotropic viral integration site 2A |
| 4.444 | GBP1 (NA) | guanylate binding protein 1, i-inducible, 67 kDa (NA-duplicate) |
| 4.445 | NFAM1 | NFAT activating protein with ITAM motif 1 |
| 4.446 | NA | NA (gene identified by probe set 242874_at) |
| 4.447 | ATP5J | ATP synthase, H+ transporting, mitochondrial F0 complex, |
| | | subunit F6 |
| 4.448 | NAV1 (NA) | neuron navigator 1 (NA-duplicate) |
| 4.449 | IGLC2 (NA) | immunoglobulin λ constant 2 (Kern-Oz-marker) (NA-duplicate) |
| 4.450 | CYLD | cylindromatosis (turban tumor syndrome) |
| 4.451 | GIMAP6 | GTPase, IMAP family member 6 |
| 4.452 | MFAP4 | microfibrillar-associated protein 4 |
| 4.453 | TUBB2B | tubulin, beta 2B |
| 4.454 | NELL2 | NEL-like 2 (chicken) |
| 4.455 | NA | NA |
| 4.456 | IL1RN | interleukin 1 receptor antagonist |
| 4.457 | KIAA1211 (NA) | NA (duplicate) |
| 4.458 | SYK | spleen tyrosine kinase |
| 4.459 | ADAMDEC1 | ADAM-like, decysin 1 |
| 4.460 | AOC3 | amine oxidase, copper containing 3 (vascular adhesion protein 1) |
| 4.461 | SAMHD1 | SAM domain and HD domain 1 |
| 4.462 | CXCL14 (NA) | chemokine (C—X—C motif) ligand 14 (NA-duplicate) |
| 4.463 | SLC22A3 | solute carrier family 22 (extraneuronal monoamine transporter), |
| | | member 3 |
| 4.464 | IL1R1 (NA) | interleukin 1 receptor, type I (NA-duplicate) |
| 4.465 | IGLV3-25 | immunoglobulin lambda variable 3-25 |
| 4.466 | NA | NA (gene identified by probe set 1556185_a_at) |
| 4.467 | RAB11FIP1 | RAB11 family interacting protein 1 (class I) |
| 4.468 | PER2 | period homolog 2 (Drosophila) |
| 4.469 | TTL | tubulin tyrosine ligase |
| 4.470 | SIAHBP1 | NA |
| 4.471 | LAPTM5 | lysosomal associated multispanning membrane protein 5 |
| 4.472 | FLJ22536 | NA |
| 4.473 | RP6-213H19.1 | NA |
| 4.474 | NA | NA (gene identified by probe set 235804_at) |
| 4.475 | NCF4 | neutrophil cytosolic factor 4, 40 kDa |
| 4.476 | EPSTI1 | epithelial stromal interaction 1 (breast) |
| 4.477 | LOC441212 | NA |
| 4.478 | ANK3 | ankyrin 3, node of Ranvier (ankyrin G) |
| 4.479 | PCDH9 | protocadherin 9 |
| 4.480 | C21orf86 | chromosome 21 open reading frame 86 |
| 4.481 | DHRS9 | dehydrogenase/reductase (SDR family) member 9 |
| 4.482 | ARHGAP25 | Rho GTPase activating protein 25 |
| 4.483 | TRAF4 | TNF receptor-associated factor 4 |
| 4.484 | LST1 | leukcocyte specific transcript 1 |
| 4.485 | PALMD | palmdelphin |
| 4.486 | TAP1 | transporter 1, ATP-binding cassette, sub-family B (MDR/TAP) |
| 4.487 | MSX2 | msh homeobox homolog 2 (Drosophila) |
| 4.488 | SIRPG | signal-regulatory protein gamma |

NA = not applicable

In the tables listed herein the given gene may be listed more than once, for example as a specific gene or as a gene cluster. Table 4 above is generated from the probe sets listed in Table 4A below. There are often multiple probe sets for each gene and thus where more than one probe set has been used to identify a particular gene then the gene appears more than once in Table 4. An attempt has been made to remove the duplication by scoring through genes that appear more than once in Table 4.

In one aspect the invention provides across the various embodiments herein one or more genes selected from Table 1, 2, 3, 4, 7, 9, 11, 12 and/or 13 including combinations thereof. In a further aspect the invention provides all the genes of Table 1, 2, 3, 4, 7, 9, 11, 12 or 13 or indeed combinations thereof.

In a further aspect the invention provides at least 10% of the genes listed in Table 1, 2, 3, 4, 7, 9, 11, 12 and/or 13, for example at least 40%, 50%, 60% or 70% such as 80%, 90% or 100% thereof.

One or more genes includes 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-105, 106-110, 111-115, 116-120, 121-125, 126-130, 131-135, 136-140, 141-145, 146-150, 151-155, 156-160, 161-165, 166-170, 171-175, 176-180, 181-185, 186-190, 191-195, 196-200, 201-205, 206-210, 211-215, 216-220, 221-225, 226-230, 231-235, 236-240, 241-245, 246-250, 251-255, 256-260, 261-265, 266-270, 271-275, 276-280, 281-285, 286-290, 291-295, 296-300, 301-305, 306-310, 311-315, 316-320, 321-325, 326-330, 331-335, 336-340, 341-345, 346-350, 351-355, 356-360, 361-365, 366-370, 371-375, 376-380, 381-385, 386-390, 391-395, 396-400, 401-405, 406-410, 411-415, 416-420, 421-425, 426-430, 431-435, 436-440, 441-445, 446-450, 451-455, 456-460, 461-465, 466-470, 471-475, 476-480, as appropriate and combinations thereof.

In one or more aspects the invention provides an embodiment as described in any one of paragraphs 1 to 430 below.

1) Thus the invention may employ one or more genes from Table 4.

2) In another aspect the invention employs one or more genes according to paragraph 1, wherein the gene has the symbol FLJ20647, optionally in combination with one or more genes labeled as 4.2 to 4.488 identified in Table 4.

3) In another aspect the invention employs one or more genes according to paragraph 1 or 2, wherein the gene has the NAV1, optionally in combination with one or more genes labeled as 4.3 to 4.488 identified in Table 4.

4) In another aspect the invention employs one or more genes according to any one of paragraphs 1-3, wherein the gene has the symbol GPR171, optionally in combination with one or more genes labeled as 4.4 to 4.488 identified in Table 4.

5) In another aspect the invention employs one or more genes according to any one of paragraphs 1-4, wherein the gene has the symbol CCL14, optionally in combination with one or more genes labeled as 4.5 to 4.488 identified in Table 4.

6) In another aspect the invention employs one or more genes according to any one of paragraphs 1-5, wherein the gene has the symbol C1S, optionally in combination with one or more genes labeled as 4.6 to 4.488 identified in Table 4.

7) In another aspect the invention employs one or more genes according to any one of paragraphs 1-6, wherein the gene has the symbol CXCL2, optionally in combination with one or more genes labeled as 4.7 to 4.488 identified in Table 4.

8) In another aspect the invention employs one or more genes according to any one of paragraphs 1-7, wherein the gene has the symbol TRBV3-1, optionally in combination with one or more genes labeled as 4.8 to 4.488 identified in Table 4.

9) In another aspect the invention employs one or more genes according to any one of paragraphs 1-8, wherein the gene has the symbol TRDV2, optionally in combination with one or more genes labeled as 4.9 to 4.488 identified in Table 4.

10) In another aspect the invention employs one or more genes according to any one of paragraphs 1-9, wherein the gene has the symbol RUFY3, optionally in combination with one or more genes labeled as 4.10 to 4.488 identified in Table 4.

11) In another aspect the invention employs one or more genes according to any one of paragraphs 1-10, wherein the gene has the symbol DOCK8, optionally in combination with one or more genes labeled as 4.11 to 4.488 identified in Table 4.

12) In another aspect the invention employs one or more genes according to any one of paragraphs 1-11, wherein the gene has the symbol GCH1, optionally in combination with one or more genes labeled as 4.12 to 4.488 identified in Table 4.

13) In another aspect the invention employs one or more genes according to any one of paragraphs 1-12, wherein the gene has the symbol CENTD3, optionally in combination with one or more genes labeled as 4.13 to 4.488 identified in Table 4.

14) In another aspect the invention employs one or more genes according to any one of paragraphs 1-13, wherein the gene has the symbol ACSL5, optionally in combination with one or more genes labeled as 4.14 to 4.488 identified in Table 4.

15) In another aspect the invention employs one or more genes according to any one of paragraphs 1-14, wherein the gene has the symbol AMICA1, optionally in combination with one or more genes labeled as 4.15 to 4.488 identified in Table 4.

16) In another aspect the invention employs one or more genes according to any one of paragraphs 1-15, wherein the gene has the symbol IL2RG, optionally in combination with one or more genes labeled as 4.16 to 4.488 identified in Table 4.

17) In another aspect the invention employs one or more genes according to any one of paragraphs 1-16, wherein the gene has the symbol TNFAIP3, optionally in combination with one or more genes labeled as 4.17 to 4.488 identified in Table 4.

18) In another aspect the invention employs one or more genes according to any one of paragraphs 1-17, wherein the gene has the symbol PSCDBP, optionally in combination with one or more genes labeled as 4.18 to 4.488 identified in Table 4.

19) In another aspect the invention employs one or more genes according to any one of paragraphs 1-18, wherein the gene has the symbol ESR1, optionally in combination with one or more genes labeled as 4.19 to 4.488 identified in Table 4.

20) In another aspect the invention employs one or more genes according to any one of paragraphs 1-19, wherein the gene has the symbol TRBC1, optionally in combination with one or more genes labeled as 4.20 to 4.488 identified in Table 4.

21) In another aspect the invention employs one or more genes according to any one of paragraphs 1-20, wherein the gene has the symbol CD52, optionally in combination with one or more genes labeled as 4.21 to 4.488 identified in Table 4.

22) In another aspect the invention employs one or more genes according to any one of paragraphs 1-21, wherein the gene has the symbol LOC442535, optionally in combination with one or more genes labeled as 4.22 to 4.488 identified in Table 4.

23) In another aspect the invention employs one or more genes according to any one of paragraphs 1-22, wherein the gene has the symbol TRBV19, optionally in combination with one or more genes labeled as 4.23 to 4.488 identified in Table 4.

24) In another aspect the invention employs one or more genes according to any one of paragraphs 1-23, wherein the gene has the symbol IL7R, optionally in combination with one or more genes labeled as 4.24 to 4.488 identified in Table 4.

25) In another aspect the invention employs one or more genes according to any one of paragraphs 1-24, wherein the gene has the symbol TRAC, optionally in combination with one or more genes labeled as 4.25 to 4.488 identified in Table 4.

26) In another aspect the invention employs one or more genes according to any one of paragraphs 1-25, wherein the gene has the symbol NCF2, optionally in combination with one or more genes labeled as 4.26 to 4.488 identified in Table 4.

27) In another aspect the invention employs one or more genes according to any one of paragraphs 1-26, wherein the gene has the symbol LOC92689, optionally in combination with one or more genes labeled as 4.27 to 4.488 identified in Table 4.

28) In another aspect the invention employs one or more genes according to any one of paragraphs 1-27, wherein the gene has the symbol GZMK, optionally in combination with one or more genes labeled as 4.28 to 4.488 identified in Table 4.

29) In another aspect the invention employs one or more genes according to any one of paragraphs 1-28, wherein the gene is the one identified by probe set 235831, optionally in combination with one or more genes labeled as 4.29 to 4.488 identified in Table 4.

30) In another aspect the invention employs one or more genes according to any one of paragraphs 1-29, wherein the gene has the symbol RAB34, optionally in combination with one or more genes labeled as 4.30 to 4.488 identified in Table 4.

31) In another aspect the invention employs one or more genes according to any one of paragraphs 1-30, wherein the gene has the symbol DPT, optionally in combination with one or more genes labeled as 4.31 to 4.488 identified in Table 4.

32) In another aspect the invention employs one or more genes according to any one of paragraphs 1-31, wherein the gene has the symbol PVT1, optionally in combination with one or more genes labeled as 4.32 to 4.488 identified in Table 4.

33) In another aspect the invention employs one or more genes according to any one of paragraphs 1-32, wherein the gene has the symbol TRGC2, optionally in combination with one or more genes labeled as 4.33 to 4.488 identified in Table 4.

34) In another aspect the invention employs one or more genes according to any one of paragraphs 1-33, wherein the gene has the symbol GIMAP5, optionally in combination with one or more genes labeled as 4.34 to 4.488 identified in Table 4.

35) In another aspect the invention employs one or more genes according to any one of paragraphs 1-34, wherein the gene has the symbol CD52, optionally in combination with one or more genes labeled as 4.35 to 4.488 identified in Table 4.

36) In another aspect the invention employs one or more genes according to any one of paragraphs 1-35, wherein the gene has the symbol CD3D, optionally in combination with one or more genes labeled as 4.36 to 4.488 identified in Table 4.

37) In another aspect the invention employs one or more genes according to any one of paragraphs 1-36, wherein the gene has the symbol TMEM132C, optionally in combination with one or more genes labeled as 4.37 to 4.488 identified in Table 4.

38) In another aspect the invention employs one or more genes according to any one of paragraphs 1-37, wherein the gene has the symbol NFKBIA, optionally in combination with one or more genes labeled as 4.38 to 4.488 identified in Table 4.

39) In another aspect the invention employs one or more genes according to any one of paragraphs 1-38, wherein the gene has the symbol TRA@, optionally in combination with one or more genes labeled as 4.39 to 4.488 identified in Table 4.

40) In another aspect the invention employs one or more genes according to any one of paragraphs 1-39, wherein the gene has the symbol TRAT1, optionally in combination with one or more genes labeled as 4.41 to 4.488 identified in Table 4.

41) In another aspect the invention employs one or more genes according to any one of paragraphs 1-40, wherein the gene has the symbol RAB34, optionally in combination with one or more genes labeled as 4.42 to 4.488 identified in Table 4.

42) In another aspect the invention employs one or more genes according to any one of paragraphs 1-41, wherein the gene has the symbol CD69, optionally in combination with one or more genes labeled as 4.43 to 4.488 identified in Table 4.

43) In another aspect the invention employs one or more genes according to any one of paragraphs 1-42, wherein the gene has the symbol DOCK8 optionally in combination with one or more genes labeled as 4.44 to 4.488 identified in Table 4.

44) In another aspect the invention employs one or more genes according to any one of paragraphs 1-43, wherein the gene has the symbol IRF8 optionally in combination with one or more genes labeled as 4.46 to 4.488 identified in Table 4.

45) In another aspect the invention employs one or more genes according to any one of paragraphs 144, wherein the gene has the symbol KLRB1 optionally in combination with one or more genes labeled as 4.47 to 4.488 identified in Table 4.

46) In another aspect the invention employs one or more genes according to any one of paragraphs 1-45, wherein the gene is identifiable by probe set number 236280_at, optionally in combination with one or more genes labeled as 4.48 to 4.488 identified in Table 4.

47) In another aspect the invention employs one or more genes according to any one of paragraphs 1-46, wherein the gene has the symbol ITGA3, optionally in combination with one or more genes labeled as 4.49 to 4.488 identified in Table 4.

48) In another aspect the invention employs one or more genes according to any one of paragraphs 1-47, wherein the gene has the symbol MAP3K8, optionally in combination with one or more genes labeled as 4.50 to 4.488 identified in Table 4.

49) In another aspect the invention employs one or more genes according to any one of paragraphs 1-48, wherein the gene has the symbol Clorf162, optionally in combination with one or more genes labeled as 4.51 to 4.488 identified in Table 4.

50) In another aspect the invention employs one or more genes according to any one of paragraphs 1-49, wherein the gene has the symbol UBD, optionally in combination with one or more genes labeled as 4.52 to 4.488 identified in Table 4.

51) In another aspect the invention employs one or more genes according to any one of paragraphs 1-50, wherein the gene has the symbol TRGV9, optionally in combination with one or more genes labeled as 4.54 to 4.488 identified in Table 4.

52) In another aspect the invention employs one or more genes according to any one of paragraphs 1-51, wherein the gene has the symbol NAV1, optionally in combination with one or more genes labeled as 4.54 to 4.488 identified in Table 4.

53) In another aspect the invention employs one or more genes according to any one of paragraphs 1-51, wherein the gene has the symbol ARHGAP9, optionally in combination with one or more genes labeled as 4.55 to 4.488 identified in Table 4.

54) In another aspect the invention employs one or more genes according to any one of paragraphs 1-53, wherein the gene has the symbol TIFA, optionally in combination with one or more genes labeled as 4.56 to 4.488 identified in Table 4.

55) In another aspect the invention employs one or more genes according to any one of paragraphs 1-54, wherein the gene is identified by probe number 1569942 at, optionally in combination with one or more genes labeled as 4.58 to 4.488 identified in Table 4.

56) In another aspect the invention employs one or more genes according to any one of paragraphs 1-55, wherein the gene has the symbol GIMAP4, optionally in combination with one or more genes labeled as 4.59 to 4.488 identified in Table 4.

57) In another aspect the invention employs one or more genes according to any one of paragraphs 1-56, wherein the gene has the symbol HCLS1, optionally in combination with one or more genes labeled as 4.60 to 4.488 identified in Table 4.

58) In another aspect the invention employs one or more genes according to any one of paragraphs 1-57, wherein the gene has the symbol PRKCH, optionally in combination with one or more genes labeled as 4.61 to 4.488 identified in Table 4.

59) In another aspect the invention employs one or more genes according to any one of paragraphs 1-58, wherein the gene has the symbol STAT4, optionally in combination with one or more genes labeled as 4.62 to 4.488 identified in Table 4.

60) In another aspect the invention employs one or more genes according to any one of paragraphs 1-59, wherein the gene has the symbol HLA-DQA1, optionally in combination with one or more genes labeled as 4.63 to 4.488 identified in Table 4.

61) In another aspect the invention employs one or more genes according to any one of paragraphs 1-60, wherein the gene has the symbol ADRB2, optionally in combination with one or more genes labeled as 4.64 to 4.488 identified in Table 4.

62) In another aspect the invention employs one or more genes according to any one of paragraphs 1-61, wherein the gene is identifiable by probe set number 239237_at, optionally in combination with one or more genes labeled as 4.65 to 4.488 identified in Table 4.

63) In another aspect the invention employs one or more genes according to any one of paragraphs 1-62, wherein the gene has the symbol CTSW, optionally in combination with one or more genes labeled as 4.66 to 4.488 identified in Table 4.

64) In another aspect the invention employs one or more genes according to any one of paragraphs 1-63, wherein the gene has the symbol MYH11, optionally in combination with one or more genes labeled as 4.67 to 4.488 identified in Table 4.

65) In another aspect the invention employs one or more genes according to any one of paragraphs 1-64, wherein the gene has the symbol GIMAP6, optionally in combination with one or more genes labeled as 4.68 to 4.488 identified in Table 4.

66) In another aspect the invention employs one or more genes according to any one of paragraphs 1-65, wherein the gene has the symbol HLA-DQB1, optionally in combination with one or more genes labeled as 4.69 to 4.488 identified in Table 4.

67) In another aspect the invention employs one or more genes according to any one of paragraphs 1-66, wherein the gene has the symbol CD8A, optionally in combination with one or more genes labeled as 4.70 to 4.488 identified in Table 4.

68) In another aspect the invention employs one or more genes according to any one of paragraphs 1-67, wherein the gene has the symbol TNFAIP3, optionally in combination with one or more genes labeled as 4.71 to 4.488 identified in Table 4.

69) In another aspect the invention employs one or more genes according to any one of paragraphs 1-68, wherein the gene has the symbol CP, optionally in combination with one or more genes labeled as 4.72 to 4.488 identified in Table 4.

70) In another aspect the invention employs one or more genes according to any one of paragraphs 1-69, wherein the gene has the symbol SMOC2, optionally in combination with one or more genes labeled as 4.73 to 4.488 identified in Table 4.

71) In another aspect the invention employs one or more genes according to any one of paragraphs 1-70, wherein the gene has the symbol C20orf24, optionally in combination with one or more genes labeled as 4.74 to 4.488 identified in Table 4.

72) In another aspect the invention employs one or more genes according to any one of paragraphs 1-71, wherein the gene has the symbol C16orf54, optionally in combination with one or more genes labeled as 4.75 to 4.488 identified in Table 4.

73) In another aspect the invention employs one or more genes according to any one of paragraphs 1-72, wherein the gene has the symbol CD2, optionally in combination with one or more genes labeled as 4.76 to 4.488 identified in Table 4.

74) In another aspect the invention employs one or more genes according to any one of paragraphs 1-73, wherein the gene has the symbol SLIT3, optionally in combination with one or more genes labeled as 4.77 to 4.488 identified in Table 4.

75) In another aspect the invention employs one or more genes according to any one of paragraphs 1-74, wherein the gene has the symbol BAALC, optionally in combination with one or more genes labeled as 4.78 to 4.488 identified in Table 4.

76) In another aspect the invention employs one or more genes according to any one of paragraphs 1-75, wherein the gene has the symbol TRIB3, optionally in combination with one or more genes labeled as 4.79 to 4.488 identified in Table 4.

77) In another aspect the invention employs one or more genes according to any one of paragraphs 1-76, wherein the gene has the symbol LOC440160, optionally in combination with one or more genes labeled as 4.80 to 4.488 identified in Table 4.

78) In another aspect the invention employs one or more genes according to any one of paragraphs 1-77, wherein the gene has the symbol C6orf190, optionally in combination with one or more genes labeled as 4.81 to 4.488 identified in Table 4.

79) In another aspect the invention employs one or more genes according to any one of paragraphs 1-78, wherein the gene has the symbol TAGAP, optionally in combination with one or more genes labeled as 4.82 to 4.488 identified in Table 4.

80) In another aspect the invention employs one or more genes according to any one of paragraphs 1-79, wherein the gene has the symbol FAM92A1, optionally in combination with one or more genes labeled as 4.83 to 4.488 identified in Table 4.

81) In another aspect the invention employs one or more genes according to any one of paragraphs 1-80, wherein the gene has the symbol PSTPIP2, optionally in combination with one or more genes labeled as 4.84 to 4.488 identified in Table 4.

82) In another aspect the invention employs one or more genes according to any one of paragraphs 1-81, wherein the gene has the symbol PTPRC, optionally in combination with one or more genes labeled as 4.85 to 4.488 identified in Table 4.

83) In another aspect the invention employs one or more genes according to any one of paragraphs 1-82, wherein the gene has the symbol HLA-DRA, optionally in combination with one or more genes labeled as 4.86 to 4.488 identified in Table 4.

84) In another aspect the invention employs one or more genes according to any one of paragraphs 1-83, wherein the gene has the symbol EFCAB2, optionally in combination with one or more genes labeled as 4.87 to 4.488 identified in Table 4.

85) In another aspect the invention employs one or more genes according to any one of paragraphs 1-84, wherein the gene has the symbol TNFAIP8, optionally in combination with one or more genes labeled as 4.88 to 4.488 identified in Table 4.

86) In another aspect the invention employs one or more genes according to any one of paragraphs 1-85, wherein the gene has the symbol SLIC1, optionally in combination with one or more genes labeled as 4.89 to 4.488 identified in Table 4.

87) In another aspect the invention employs one or more genes according to any one of paragraphs 1-86, wherein the gene has the symbol CD1C, optionally in combination with one or more genes labeled as 4.90 to 4.488 identified in Table 4.

88) In another aspect the invention employs one or more genes according to any one of paragraphs 1-87, wherein the gene has the symbol TRAF31P3, optionally in combination with one or more genes labeled as 4.91 to 4.488 identified in Table 4.

89) In another aspect the invention employs one or more genes according to any one of paragraphs 1-88, wherein the gene has the symbol IGJ, optionally in combination with one or more genes labeled as 4.96 to 4.488 identified in Table 4.

90) In another aspect the invention employs one or more genes according to any one of paragraphs 1-89, wherein the gene has the symbol PLEK, optionally in combination with one or more genes labeled as 4.98 to 4.488 identified in Table 4.

91) In another aspect the invention employs one or more genes according to any one of paragraphs 1-90, wherein the gene has the symbol TMEM44, optionally in combination with one or more genes labeled as 4.100 to 4.488 identified in Table 4.

92) In another aspect the invention employs one or more genes according to any one of paragraphs 1-91, wherein the gene has the symbol EBI2, optionally in combination with one or more genes labeled as 4.101 to 4.488 identified in Table 4.

93) In another aspect the invention employs one or more genes according to any one of paragraphs 1-92, wherein the gene has the symbol SAMSN1, optionally in combination with one or more genes labeled as 4.102 to 4.488 identified in Table 4.

94) In another aspect the invention employs one or more genes according to any one of paragraphs 1-93, wherein the gene has the symbol KIAA1794, optionally in combination with one or more genes labeled as 4.103 to 4.488 identified in Table 4.

95) In another aspect the invention employs one or more genes according to any one of paragraphs 1-94, wherein the gene has the symbol ALDH2, optionally in combination with one or more genes labeled as 4.104 to 4.488 identified in Table 4.

96) In another aspect the invention employs one or more genes according to any one of paragraphs 1-95, wherein the gene has the symbol CDC42SE2, optionally in combination with one or more genes labeled as 4.105 to 4.488 identified in Table 4.

97) In another aspect the invention employs one or more genes according to any one of paragraphs 1-96, wherein the gene has the symbol GFRA1, optionally in combination with one or more genes labeled as 4.106 to 4.488 identified in Table 4.

98) In another aspect the invention employs one or more genes according to any one of paragraphs 1-97, wherein the gene has the symbol ITK, optionally in combination with one or more genes labeled as 4.107 to 4.488 identified in Table 4.

99) In another aspect the invention employs one or more genes according to any one of paragraphs 1-98, wherein the gene has the symbol GIMAP7, optionally in combination with one or more genes labeled as 4.109 to 4.488 identified in Table 4.

100) In another aspect the invention employs one or more genes according to any one of paragraphs 1-99, wherein the gene has the symbol FLJ20273, optionally in combination with one or more genes labeled as 4.110 to 4.488 identified in Table 4.

101) In another aspect the invention employs one or more genes according to any one of paragraphs 1-100, wherein the gene has the symbol PTPN6, optionally in combination with one or more genes labeled as 4.111 to 4.488 identified in Table 4.

102) In another aspect the invention employs one or more genes according to any one of paragraphs 1-101, wherein the gene has the symbol PTGER3, optionally in combination with one or more genes labeled as 4.112 to 4.488 identified in Table 4.

103) In another aspect the invention employs one or more genes according to any one of paragraphs 1-102, wherein the gene has the symbol RAI2, optionally in combination with one or more genes labeled as 4.113 to 4.488 identified in Table 4.

104) In another aspect the invention employs one or more genes according to any one of paragraphs 1-103, wherein the gene has the symbol LGALS2, optionally in combination with one or more genes labeled as 4.114 to 4.488 identified in Table 4.

105) In another aspect the invention employs one or more genes according to any one of paragraphs 1-104, wherein the gene has the symbol HMOX1, optionally in combination with one or more genes labeled as 4.115 to 4.488 identified in Table 4.

106) In another aspect the invention employs one or more genes according to any one of paragraphs 1-105, wherein the gene is identifiable by probe set number 227995 at, optionally in combination with one or more genes labeled as 4.116 to 4.488 identified in Table 4.

107) In another aspect the invention employs one or more genes according to any one of paragraphs 1-106, wherein the gene has the symbol ZNFN1A1, optionally in combination with one or more genes labeled as 4.117 to 4.488 identified in Table 4.

108) In another aspect the invention employs one or more genes according to any one of paragraphs 1-107, wherein the gene has the symbol CSF2RB, optionally in combination with one or more genes labeled as 4.118 to 4.488 identified in Table 4.

109) In another aspect the invention employs one or more genes according to any one of paragraphs 1-108, wherein the gene has the symbol PCSK5, optionally in combination with one or more genes labeled as 4.119 to 4.488 identified in Table 4.

110) In another aspect the invention employs one or more genes according to any one of paragraphs 1-109, wherein the gene has the symbol CCDC69, optionally in combination with one or more genes labeled as 4.120 to 4.488 identified in Table 4.

111) In another aspect the invention employs one or more genes according to any one of paragraphs 1-110, wherein the gene has the symbol CDC42SE2, optionally in combination with one or more genes labeled as 4.121 to 4.488 identified in Table 4.

112) In another aspect the invention employs one or more genes according to any one of paragraphs 1-111, wherein the gene has the symbol GZMA, optionally in combination with one or more genes labeled as 4.122 to 4.488 identified in Table 4.

113) In another aspect the invention employs one or more genes according to any one of paragraphs 1-112, wherein the gene has the symbol C3, optionally in combination with one or more genes labeled as 4.123 to 4.488 identified in Table 4.

114) In another aspect the invention employs one or more genes according to any one of paragraphs 1-113, wherein the gene has the symbol C15orf48, optionally in combination with one or more genes labeled as 4.125 to 4.488 identified in Table 4.

115) In another aspect the invention employs one or more genes according to any one of paragraphs 1-114, wherein the gene has the symbol RARRES3, optionally in combination with one or more genes labeled as 4.126 to 4.488 identified in Table 4.

116) In another aspect the invention employs one or more genes according to any one of paragraphs 1-115, wherein the gene has the symbol LOC283537, optionally in combination with one or more genes labeled as 4.127 to 4.488 identified in Table 4.

117) In another aspect the invention employs one or more genes according to any one of paragraphs 1-116, wherein the gene has the symbol CXCL12, optionally in combination with one or more genes labeled as 4.129 to 4.488 identified in Table 4.

118) In another aspect the invention employs one or more genes according to any one of paragraphs 1-117, wherein the gene is identifiable by probe set number 231882_at, optionally in combination with one or more genes labeled as 4.130 to 4.488 identified in Table 4.

119) In another aspect the invention employs one or more genes according to any one of paragraphs 1-118, wherein the gene has the symbol SOD2, optionally in combination with one or more genes labeled as 4.131 to 4.488 identified in Table 4.

120) In another aspect the invention employs one or more genes according to any one of paragraphs 1-119, wherein the gene has the symbol CTSS, optionally in combination with one or more genes labeled as 4.132 to 4.488 identified in Table 4.

121) In another aspect the invention employs one or more genes according to any one of paragraphs 1-120, wherein the gene has the symbol CTBP2, optionally in combination with one or more genes labeled as 4.133 to 4.488 identified in Table 4.

122) In another aspect the invention employs one or more genes according to any one of paragraphs 1-121, wherein the gene has the symbol BCL11B, optionally in combination with one or more genes labeled as 4.134 to 4.488 identified in Table 4.

123) In another aspect the invention employs one or more genes according to any one of paragraphs 1-122, wherein the gene has the symbol CCL22, optionally in combination with one or more genes labeled as 4.135 to 4.488 identified in Table 4.

124) In another aspect the invention employs one or more genes according to any one of paragraphs 1-123, wherein the gene has the symbol ACSL5, optionally in combination with one or more genes labeled as 4.136 to 4.488 identified in Table 4.

125) In another aspect the invention employs one or more genes according to any one of paragraphs 1-124, wherein the gene has the symbol DOC1, optionally in combination with one or more genes labeled as 4.137 to 4.488 identified in Table 4.

126) In another aspect the invention employs one or more genes according to any one of paragraphs 1-125, wherein the gene has the symbol SLC31A2, optionally in combination with one or more genes labeled as 4.138 to 4.488 identified in Table 4.

127) In another aspect the invention employs one or more genes according to any one of paragraphs 1-126, wherein the gene has the symbol POPDC3, optionally in combination with one or more genes labeled as 4.139 to 4.488 identified in Table 4.

128) In another aspect the invention employs one or more genes according to any one of paragraphs 1-127, wherein the gene has the symbol SQRDL, optionally in combination with one or more genes labeled as 4.141 to 4.488 identified in Table 4.

129) In another aspect the invention employs one or more genes according to any one of paragraphs 1-128, wherein the gene has the symbol RASGEF1B, optionally in combination with one or more genes labeled as 4.142 to 4.488 identified in Table 4.

130) In another aspect the invention employs one or more genes according to any one of paragraphs 1-129, wherein the gene has the symbol FGL2, optionally in combination with one or more genes labeled as 4.143 to 4.488 identified in Table 4.

131) In another aspect the invention employs one or more genes according to any one of paragraphs 1-130, wherein the gene has the symbol C10orf128, optionally in combination with one or more genes labeled as 4.144 to 4.488 identified in Table 4.

132) In another aspect the invention employs one or more genes according to any one of paragraphs 1-131, wherein the gene has the symbol IL10RA, optionally in combination with one or more genes labeled as 4.145 to 4.488 identified in Table 4.

133) In another aspect the invention employs one or more genes according to any one of paragraphs 1-132, wherein the gene has the symbol EGFL6, optionally in combination with one or more genes labeled as 4.146 to 4.488 identified in Table 4.

134) In another aspect the invention employs one or more genes according to any one of paragraphs 1-133, wherein the gene has the symbol IL18, optionally in combination with one or more genes labeled as 4.147 to 4.488 identified in Table 4.

135) In another aspect the invention employs one or more genes according to any one of paragraphs 1-134, wherein the gene has the symbol ARHGAP30, optionally in combination with one or more genes labeled as 4.148 to 4.488 identified in Table 4.

136) In another aspect the invention employs one or more genes according to any one of paragraphs 1-135, wherein the gene has the symbol PALMD, optionally in combination with one or more genes labeled as 4.149 to 4.488 identified in Table 4.

137) In another aspect the invention employs one or more genes according to any one of paragraphs 1-136, wherein the gene has the symbol RASSF5, optionally in combination with one or more genes labeled as 4.150 to 4.488 identified in Table 4.

138) In another aspect the invention employs one or more genes according to any one of paragraphs 1-137, wherein the gene has the symbol GATA3, optionally in combination with one or more genes labeled as 4.151 to 4.488 identified in Table 4.

139) In another aspect the invention employs one or more genes according to any one of paragraphs 1-138, wherein the gene has the symbol DKFZP564O0823, optionally in combination with one or more genes labeled as 4.152 to 4.488 identified in Table 4.

140) In another aspect the invention employs one or more genes according to any one of paragraphs 1-139, wherein the gene has the symbol TXNIP, optionally in combination with one or more genes labeled as 4.154 to 4.488 identified in Table 4.

141) In another aspect the invention employs one or more genes according to any one of paragraphs 1-140, wherein the gene has the symbol DTX4, optionally in combination with one or more genes labeled as 4.155 to 4.488 identified in Table 4.

142) In another aspect the invention employs one or more genes according to any one of paragraphs 1-141, wherein the gene has the symbol DARC, optionally in combination with one or more genes labeled as 4.156 to 4.488 identified in Table 4.

143) In another aspect the invention employs one or more genes according to any one of paragraphs 1-142, wherein the gene has the symbol RNASE6, optionally in combination with one or more genes labeled as 4.157 to 4.488 identified in Table 4.

144) In another aspect the invention employs one or more genes according to any one of paragraphs 1-143, wherein the gene has the symbol CD86, optionally in combination with one or more genes labeled as 4.158 to 4.488 identified in Table 4.

145) In another aspect the invention employs one or more genes according to any one of paragraphs 1-144, wherein the gene has the symbol ZFP36, optionally in combination with one or more genes labeled as 4.159 to 4.488 identified in Table 4.

146) In another aspect the invention employs one or more genes according to any one of paragraphs 1-145, wherein the gene has the symbol BASP1, optionally in combination with one or more genes labeled as 4.160 to 4.488 identified in Table 4.

147) In another aspect the invention employs one or more genes according to any one of paragraphs 1-146, wherein the gene has the symbol CKAP1, optionally in combination with one or more genes labeled as 4.161 to 4.488 identified in Table 4.

148) In another aspect the invention employs one or more genes according to any one of paragraphs 1-147, wherein the gene has the symbol HCP5, optionally in combination with one or more genes labeled as 4.162 to 4.488 identified in Table 4.

149) In another aspect the invention employs one or more genes according to any one of paragraphs 1-148, wherein the gene has the symbol GRB14, optionally in combination with one or more genes labeled as 4.163 to 4.488 identified in Table 4.

150) In another aspect the invention employs one or more genes according to any one of paragraphs 1-149, wherein the gene has the symbol GJA7, optionally in combination with one or more genes labeled as 4.164 to 4.488 identified in Table 4.

151) In another aspect the invention employs one or more genes according to any one of paragraphs 1-150, wherein the gene has the symbol FLJ14054, optionally in combination with one or more genes labeled as 4.165 to 4.488 identified in Table 4.

152) In another aspect the invention employs one or more genes according to any one of paragraphs 1-151, wherein the gene has the symbol VNN1, optionally in combination with one or more genes labeled as 4.166 to 4.488 identified in Table 4.

153) In another aspect the invention employs one or more genes according to any one of paragraphs 1-152, wherein the gene has the symbol ADCY7, optionally in combination with one or more genes labeled as 4.167 to 4.488 identified in Table 4.

154) In another aspect the invention employs one or more genes according to any one of paragraphs 1-153, wherein the gene has the symbol MS4A6A, optionally in combination with one or more genes labeled as 4.168 to 4.488 identified in Table 4.

155) In another aspect the invention employs one or more genes according to any one of paragraphs 1-154, wherein the gene has the symbol CPA3, optionally in combination with one or more genes labeled as 4.169 to 4.488 identified in Table 4.

156) In another aspect the invention employs one or more genes according to any one of paragraphs 1-155, wherein the gene has the symbol PIM1, optionally in combination with one or more genes labeled as 4.170 to 4.488 identified in Table 4.

157) In another aspect the invention employs one or more genes according to any one of paragraphs 1-156, wherein the gene has the symbol CCL19, optionally in combination with one or more genes labeled as 4.171 to 4.488 identified in Table 4.

158) In another aspect the invention employs one or more genes according to any one of paragraphs 1-157, wherein the gene has the symbol SYK, optionally in combination with one or more genes labeled as 4.172 to 4.488 identified in Table 4.

159) In another aspect the invention employs one or more genes according to any one of paragraphs 1-158, wherein the gene has the symbol SIT1, optionally in combination with one or more genes labeled as 4.174 to 4.488 identified in Table 4.

160) In another aspect the invention employs one or more genes according to any one of paragraphs 1-159, wherein the gene is identifiable by probe set number 228812_at, optionally in combination with one or more genes labeled as 4.175 to 4.488 identified in Table 4.

161) In another aspect the invention employs one or more genes according to any one of paragraphs 1-160, wherein the gene has the symbol NAP1L2, optionally in combination with one or more genes labeled as 4.176 to 4.488 identified in Table 4.

162) In another aspect the invention employs one or more genes according to any one of paragraphs 1-161, wherein the gene has the symbol CCL13, optionally in combination with one or more genes labeled as 4.177 to 4.488 identified in Table 4.

163) In another aspect the invention employs one or more genes according to any one of paragraphs 1-162, wherein the gene has the symbol SLA, optionally in combination with one or more genes labeled as 4.178 to 4.488 identified in Table 4.

164) In another aspect the invention employs one or more genes according to any one of paragraphs 1-163, wherein the gene has the symbol NOD3, optionally in combination with one or more genes labeled as 4.179 to 4.488 identified in Table 4.

165) In another aspect the invention employs one or more genes according to any one of paragraphs 1-164, wherein the gene has the symbol PRKCH, optionally in combination with one or more genes labeled as 4.180 to 4.488 identified in Table 4.

166) In another aspect the invention employs one or more genes according to any one of paragraphs 1-165, wherein the gene has the symbol TRD@, optionally in combination with one or more genes labeled as 4.181 to 4.488 identified in Table 4.

167) In another aspect the invention employs one or more genes according to any one of paragraphs 1-166, wherein the gene has the symbol BAALC, optionally in combination with one or more genes labeled as 4.182 to 4.488 identified in Table 4.

168) In another aspect the invention employs one or more genes according to any one of paragraphs 1-167, wherein the gene has the symbol RP1-93H18.5, optionally in combination with one or more genes labeled as 4.183 to 4.488 identified in Table 4.

169) In another aspect the invention employs one or more genes according to any one of paragraphs 1-168, wherein the gene has the symbol FLJ20701, optionally in combination with one or more genes labeled as 4.184 to 4.488 identified in Table 4.

170) In another aspect the invention employs one or more genes according to any one of paragraphs 1-169, wherein the gene has the symbol SH3TC2, optionally in combination with one or more genes labeled as 4.185 to 4.488 identified in Table 4.

171) In another aspect the invention employs one or more genes according to any one of paragraphs 1-170, wherein the gene has the symbol CCR2, optionally in combination with one or more genes labeled as 4.186 to 4.488 identified in Table 4.

172) In another aspect the invention employs one or more genes according to any one of paragraphs 1-171, wherein the gene has the symbol CCL5, optionally in combination with one or more genes labeled as 4.187 to 4.488 identified in Table 4.

173) In another aspect the invention employs one or more genes according to any one of paragraphs 1-172, wherein the gene has the symbol HLA-DPA1, optionally in combination with one or more genes labeled as 4.189 to 4.488 identified in Table 4.

174) In another aspect the invention employs one or more genes according to any one of paragraphs 1-173, wherein the gene has the symbol PECAM1, optionally in combination with one or more genes labeled as 4.190 to 4.488 identified in Table 4.

175) In another aspect the invention employs one or more genes according to any one of paragraphs 1-174, wherein the gene has the symbol AMIGO2, optionally in combination with one or more genes labeled as 4.192 to 4.488 identified in Table 4.

176) In another aspect the invention employs one or more genes according to any one of paragraphs 1-175, wherein the gene has the symbol CLEC7A, optionally in combination with one or more genes labeled as 4.193 to 4.488 identified in Table 4.

177) In another aspect the invention employs one or more genes according to any one of paragraphs 1-176, wherein the gene has the symbol P2RY14, optionally in combination with one or more genes labeled as 4.194 to 4.488 identified in Table 4.

178) In another aspect the invention employs one or more genes according to any one of paragraphs 1-177, wherein the gene has the symbol PIK3AP1, optionally in combination with one or more genes labeled as 4.195 to 4.488 identified in Table 4.

179) In another aspect the invention employs one or more genes according to any one of paragraphs 1-178, wherein the gene has the symbol ADH1B, optionally in combination with one or more genes labeled as 4.196 to 4.488 identified in Table 4.

180) In another aspect the invention employs one or more genes according to any one of paragraphs 1-179, wherein the gene has the symbol TOP1MT, optionally in combination with one or more genes labeled as 4.197 to 4.488 identified in Table 4.

181) In another aspect the invention employs one or more genes according to any one of paragraphs 1-180, wherein the gene has the symbol CD276, optionally in combination with one or more genes labeled as 4.199 to 4.488 identified in Table 4.

182) In another aspect the invention employs one or more genes according to any one of paragraphs 1-181, wherein the gene has the symbol JAM2, optionally in combination with one or more genes labeled as 4.200 to 4.488 identified in Table 4.

183) In another aspect the invention employs one or more genes according to any one of paragraphs 1-182, wherein the gene has the symbol C1S, optionally in combination with one or more genes labeled as 4.202 to 4.488 identified in Table 4.

184) In another aspect the invention employs one or more genes according to any one of paragraphs 1-183, wherein the gene has the symbol TGFBR3, optionally in combination with one or more genes labeled as 4.203 to 4.488 identified in Table 4.

185) In another aspect the invention employs one or more genes according to any one of paragraphs 1-184, wherein the gene has the symbol ITGAL, optionally in combination with one or more genes labeled as 4.204 to 4.488 identified in Table 4.

186) In another aspect the invention employs one or more genes according to any one of paragraphs 1-185, wherein the gene has the symbol IL1R1, optionally in combination with one or more genes labeled as 4.206 to 4.488 identified in Table 4.

187) In another aspect the invention employs one or more genes according to any one of paragraphs 1-186, wherein the gene has the symbol HLA-DRB1, optionally in combination with one or more genes labeled as 4.207 to 4.488 identified in Table 4.

188) In another aspect the invention employs one or more genes according to any one of paragraphs 1-187, wherein the gene has the symbol GIMAP2, optionally in combination with one or more genes labeled as 4.208 to 4.488 identified in Table 4.

189) In another aspect the invention employs one or more genes according to any one of paragraphs 1-188, wherein the gene has the symbol ZC3H12D, optionally in combination with one or more genes labeled as 4.209 to 4.488 identified in Table 4.

190) In another aspect the invention employs one or more genes according to any one of paragraphs 1-189, wherein the gene has the symbol PCDH9, optionally in combination with one or more genes labeled as 4.210 to 4.488 identified in Table 4.

191) In another aspect the invention employs one or more genes according to any one of paragraphs 1-190, wherein the gene has the symbol SLAMF7, optionally in combination with one or more genes labeled as 4.211 to 4.488 identified in Table 4.

192) In another aspect the invention employs one or more genes according to any one of paragraphs 1-191, wherein the gene has the symbol MGC7036, optionally in combination with one or more genes labeled as 4.212 to 4.488 identified in Table 4.

193) In another aspect the invention employs one or more genes according to any one of paragraphs 1-192, wherein the gene has the symbol RGS18, optionally in combination with one or more genes labeled as 4.214 to 4.488 identified in Table 4.

194) In another aspect the invention employs one or more genes according to any one of paragraphs 1-193, wherein the gene has the symbol CD53, optionally in combination with one or more genes labeled as 4.215 to 4.488 identified in Table 4.

195) In another aspect the invention employs one or more genes according to any one of paragraphs 1-194, wherein the gene has the symbol MPEG1, optionally in combination with one or more genes labeled as 4.216 to 4.488 identified in Table 4.

196) In another aspect the invention employs one or more genes according to any one of paragraphs 1-195, wherein the gene has the symbol SSBP4, optionally in combination with one or more genes labeled as 4.217 to 4.488 identified in Table 4.

197) In another aspect the invention employs one or more genes according to any one of paragraphs 1-196, wherein the gene is identifiable by probe set number 231262_at, optionally in combination with one or more genes labeled as 4.218 to 4.488 identified in Table 4.

198) In another aspect the invention employs one or more genes according to any one of paragraphs 1-197, wherein the gene has the symbol CDH19, optionally in combination with one or more genes labeled as 4.219 to 4.488 identified in Table 4.

199) In another aspect the invention employs one or more genes according to any one of paragraphs 1-198, wherein the gene has the symbol CTBP2, optionally in combination with one or more genes labeled as 4.221 to 4.488 identified in Table 4.

200) In another aspect the invention employs one or more genes according to any one of paragraphs 1-199, wherein the gene has the symbol FAM107B, optionally in combination with one or more genes labeled as 4.222 to 4.488 identified in Table 4.

201) in another aspect the invention employs one or more genes according to any one of paragraphs 1-200, wherein the gene has the symbol IGKC, optionally in combination with one or more genes labeled as 4.223 to 4.488 identified in Table 4.

202) In another aspect the invention employs one or more genes according to any one of paragraphs 1-201, wherein the gene has the symbol ITGAM, optionally in combination with one or more genes labeled as 4.224 to 4.488 identified in Table 4.

203) In another aspect the invention employs one or more genes according to any one of paragraphs 1-202, wherein the gene has the symbol CKAP1, optionally in combination with one or more genes labeled as 4.227 to 4.488 identified in Table 4.

204) In another aspect the invention employs one or more genes according to any one of paragraphs 1-203, wherein the gene has the symbol MGC16291, optionally in combination with one or more genes labeled as 4.228 to 4.488 identified in Table 4.

205) In another aspect the invention employs one or more genes according to any one of paragraphs 1-204, wherein the gene has the symbol DDEF2, optionally in combination with one or more genes labeled as 4.229 to 4.488 identified in Table 4.

206) In another aspect the invention employs one or more genes according to any one of paragraphs 1-205, wherein the gene has the symbol TNFAIP2, optionally in combination with one or more genes labeled as 4.230 to 4.488 identified in Table 4.

207) In another aspect the invention employs one or more genes according to any one of paragraphs 1-206, wherein the gene has the symbol CXCL14, optionally in combination with one or more genes labeled as 4.231 to 4.488 identified in Table 4.

208) In another aspect the invention employs one or more genes according to any one of paragraphs 1-207, wherein the gene has the symbol CD209, optionally in combination with one or more genes labeled as 4.232 to 4.488 identified in Table 4.

209) In another aspect the invention employs one or more genes according to any one of paragraphs 1-208, wherein the gene has the symbol COL9A3, optionally in combination with one or more genes labeled as 4.233 to 4.488 identified in Table 4.

210) In another aspect the invention employs one or more genes according to any one of paragraphs 1-209, wherein the gene has the symbol ANKRD22, optionally in combination with one or more genes labeled as 4.234 to 4.488 identified in Table 4.

211) In another aspect the invention employs one or more genes according to any one of paragraphs 1-210, wherein the gene has the symbol NCKAP1L, optionally in combination with one or more genes labeled as 4.235 to 4.488 identified in Table 4.

212) In another aspect the invention employs one or more genes according to any one of paragraphs 1-211, wherein the gene has the symbol CMKOR1, optionally in combination with one or more genes labeled as 4.236 to 4.488 identified in Table 4.

213) In another aspect the invention employs one or more genes according to any one of paragraphs 1-212, wherein the gene has the symbol HLA-DRB5, optionally in combination with one or more genes labeled as 4.237 to 4.488 identified in Table 4.

214) In another aspect the invention employs one or more genes according to any one of paragraphs 1-213, wherein the gene has the symbol LCP1, optionally in combination with one or more genes labeled as 4.239 to 4.488 identified in Table 4.

215) In another aspect the invention employs one or more genes according to any one of paragraphs 1-214, wherein the gene has the symbol CXXC5, optionally in combination with one or more genes labeled as 4.240 to 4.488 identified in Table 4.

216) In another aspect the invention employs one or more genes according to any one of paragraphs 1-215, wherein the gene has the symbol GJA7, optionally in combination with one or more genes labeled as 4.241 to 4.488 identified in Table 4.

217) In another aspect the invention employs one or more genes according to any one of paragraphs 1-216, wherein the gene has the symbol FGD2, optionally in combination with one or more genes labeled as 4.242 to 4.488 identified in Table 4.

218) In another aspect the invention employs one or more genes according to any one of paragraphs 1-217, wherein the gene has the symbol MAN1A1, optionally in combination with one or more genes labeled as 4.243 to 4.488 identified in Table 4.

219) In another aspect the invention employs one or more genes according to any one of paragraphs 1-218, wherein the gene has the symbol C6orf115, optionally in combination with one or more genes labeled as 4.245 to 4.488 identified in Table 4.

220) In another aspect the invention employs one or more genes according to any one of paragraphs 1-219, wherein the gene has the symbol CXCL9, optionally in combination with one or more genes labeled as 4.247 to 4.488 identified in Table 4.

221) In another aspect the invention employs one or more genes according to any one of paragraphs 1-220, wherein the gene has the symbol NPR3, optionally in combination with one or more genes labeled as 4.248 to 4.488 identified in Table 4.

222) In another aspect the invention employs one or more genes according to any one of paragraphs 1-221, wherein the gene has the symbol FYB, optionally in combination with one or more genes labeled as 4.249 to 4.488 identified in Table 4.

223) In another aspect the invention employs one or more genes according to any one of paragraphs 1-222, wherein the gene has the symbol VCAM1, optionally in combination with one or more genes labeled as 4.250 to 4.488 identified in Table 4.

224) In another aspect the invention employs one or more genes according to any one of paragraphs 1-223, wherein the gene has the symbol FLI1, optionally in combination with one or more genes labeled as 4.251 to 4.488 identified in Table 4.

225) In another aspect the invention employs one or more genes according to any one of paragraphs 1-224, wherein the gene has the symbol CXXC5, optionally in combination with one or more genes labeled as 4.252 to 4.488 identified in Table 4.

226) In another aspect the invention employs one or more genes according to any one of paragraphs 1-225, wherein the gene has the symbol TRAM2, optionally in combination with one or more genes labeled as 4.254 to 4.488 identified in Table 4.

227) In another aspect the invention employs one or more genes according to any one of paragraphs 1-226, wherein the gene has the symbol SHC4, optionally in combination with one or more genes labeled as 4.255 to 4.488 identified in Table 4.

228) In another aspect the invention employs one or more genes according to any one of paragraphs 1-227, wherein the gene has the symbol SLC9A9, optionally in combination with one or more genes labeled as 4.256 to 4.488 identified in Table 4.

229) In another aspect the invention employs one or more genes according to any one of paragraphs 1-228, wherein the gene has the symbol PTPRC, optionally in combination with one or more genes labeled as 4.257 to 4.488 identified in Table 4.

230) In another aspect the invention employs one or more genes according to any one of paragraphs 1-229, wherein the gene has the symbol PTGER4, optionally in combination with one or more genes labeled as 4.258 to 4.488 identified in Table 4.

231) In another aspect the invention employs one or more genes according to any one of paragraphs 1-230, wherein the gene has the symbol LILRB1, optionally in combination with one or more genes labeled as 4.259 to 4.488 identified in Table 4.

232) In another aspect the invention employs one or more genes according to any one of paragraphs 1-231, wherein the gene has the symbol PRDM1, optionally in combination with one or more genes labeled as 4.261 to 4.488 identified in Table 4.

233) In another aspect the invention employs one or more genes according to any one of paragraphs 1-232, wherein the gene has the symbol ARHGAP15, optionally in combination with one or more genes labeled as 4.262 to 4.488 identified in Table 4.

234) In another aspect the invention employs one or more genes according to any one of paragraphs 1-233, wherein the gene has the symbol SLC5A3, optionally in combination with one or more genes labeled as 4.263 to 4.488 identified in Table 4.

235) In another aspect the invention employs one or more genes according to any one of paragraphs 1-234, wherein the gene has the symbol DOCK9, optionally in combination with one or more genes labeled as 4.264 to 4.488 identified in Table 4.

236) In another aspect the invention employs one or more genes according to any one of paragraphs 1-235, wherein the gene has the symbol GPSM1, optionally in combination with one or more genes labeled as 4.265 to 4.488 identified in Table 4.

237) In another aspect the invention employs one or more genes according to any one of paragraphs 1-236, wherein the gene has the symbol CCL5, optionally in combination with one or more genes labeled as 4.266 to 4.488 identified in Table 4.

238) In another aspect the invention employs one or more genes according to any one of paragraphs 1-237, wherein the gene has the symbol GLIPR1, optionally in combination with one or more genes labeled as 4.267 to 4.488 identified in Table 4.

239) In another aspect the invention employs one or more genes according to any one of paragraphs 1-238, wherein the gene has the symbol APOL3, optionally in combination with one or more genes labeled as 4.268 to 4.488 identified in Table 4.

240) In another aspect the invention employs one or more genes according to any one of paragraphs 1-239, wherein the gene has the symbol HLA-DMB, optionally in combination with one or more genes labeled as 4.269 to 4.488 identified in Table 4.

241) In another aspect the invention employs one or more genes according to any one of paragraphs 1-240, wherein the gene has the symbol SYNPO2, optionally in combination with one or more genes labeled as 4.270 to 4.488 identified in Table 4.

242) In another aspect the invention employs one or more genes according to any one of paragraphs 1-241, wherein the gene is identifiable by probe set number 221651_x_at, optionally in combination with one or more genes labeled as 4.271 to 4.488 identified in Table 4.

243) In another aspect the invention employs one or more genes according to any one of paragraphs 1-242, wherein the gene is identifiable by probe set number 231929_at, optionally in combination with one or more genes labeled as 4.273 to 4.488 identified in Table 4.

244) In another aspect the invention employs one or more genes according to any one of paragraphs 1-243, wherein the gene has the symbol CASP1, optionally in combination with one or more genes labeled as 4.274 to 4.488 identified in Table 4.

245) In another aspect the invention employs one or more genes according to any one of paragraphs 1-244, wherein the gene has the symbol PRKCQ, optionally in combination with one or more genes labeled as 4.275 to 4.488 identified in Table 4.

246) In another aspect the invention employs one or more genes according to any one of paragraphs 1-245, wherein the gene has the symbol IL1R2, optionally in combination with one or more genes labeled as 4.276 to 4.488 identified in Table 4.

247) In another aspect the invention employs one or more genes according to any one of paragraphs 1-246, wherein the gene has the symbol CARD15, optionally in combination with one or more genes labeled as 4.277 to 4.488 identified in Table 4.

248) In another aspect the invention employs one or more genes according to any one of paragraphs 1-247, wherein the gene has the symbol ARHGDIB, optionally in combination with one or more genes labeled as 4.278 to 4.488 identified in Table 4.

249) In another aspect the invention employs one or more genes according to any one of paragraphs 1-248, wherein the gene has the symbol HLA-DRB4, optionally in combination with one or more genes labeled as 4.279 to 4.488 identified in Table 4.

250) In another aspect the invention employs one or more genes according to any one of paragraphs 1-249, wherein the gene has the symbol SART2, optionally in combination with one or more genes labeled as 4.280 to 4.488 identified in Table 4.

251) In another aspect the invention employs one or more genes according to any one of paragraphs 1-250, wherein the gene has the symbol LSP1, optionally in combination with one or more genes labeled as 4.281 to 4.488 identified in Table 4.

252) In another aspect the invention employs one or more genes according to any one of paragraphs 1-251, wherein the gene has the symbol AMPD3, optionally in combination with one or more genes labeled as 4.282 to 4.488 identified in Table 4.

253) In another aspect the invention employs one or more genes according to any one of paragraphs 1-252, wherein the gene has the symbol SEMA4F, optionally in combination with one or more genes labeled as 4.283 to 4.488 identified in Table 4.

254) In another aspect the invention employs one or more genes according to any one of paragraphs 1-253, wherein the gene has the symbol ISOC1, optionally in combination with one or more genes labeled as 4.285 to 4.488 identified in Table 4.

255) In another aspect the invention employs one or more genes according to any one of paragraphs 1-254, wherein the gene has the symbol HPS3, optionally in combination with one or more genes labeled as 4.288 to 4.488 identified in Table 4.

256) In another aspect the invention employs one or more genes according to any one of paragraphs 1-255, wherein the gene has the symbol HOXB7, optionally in combination with one or more genes labeled as 4.290 to 4.488 identified in Table 4.

257) In another aspect the invention employs one or more genes according to any one of paragraphs 1-256, wherein the gene has the symbol ZNFN1A1, optionally in combination with one or more genes labeled as 4.291 to 4.488 identified in Table 4.

258) In another aspect the invention employs one or more genes according to any one of paragraphs 1-257, wherein the gene has the symbol ARHGAP9, optionally in combination with one or more genes labeled as 4.292 to 4.488 identified in Table 4.

259) In another aspect the invention employs one or more genes according to any one of paragraphs 1-258, wherein the gene has the symbol GATA2, optionally in combination with one or more genes labeled as 4.293 to 4.488 identified in Table 4.

260) In another aspect the invention employs one or more genes according to any one of paragraphs 1-259, wherein the gene has the symbol AP2B1, optionally in combination with one or more genes labeled as 4.294 to 4.488 identified in Table 4.

261) In another aspect the invention employs one or more genes according to any one of paragraphs 1-260, wherein the gene has the symbol CTSC, optionally in combination with one or more genes labeled as 4.295 to 4.488 identified in Table 4.

262) In another aspect the invention employs one or more genes according to any one of paragraphs 1-261, wherein the gene has the symbol PLK2, optionally in combination with one or more genes labeled as 4.296 to 4.488 identified in Table 4.

263) In another aspect the invention employs one or more genes according to any one of paragraphs 1-262, wherein the gene has the symbol CD4, optionally in combination with one or more genes labeled as 4.297 to 4.488 identified in Table 4.

264) In another aspect the invention employs one or more genes according to any one of paragraphs 1-263, wherein the gene has the symbol GGTA1, optionally in combination with one or more genes labeled as 4.298 to 4.488 identified in Table 4.

265) In another aspect the invention employs one or more genes according to any one of paragraphs 1-264, wherein the gene has the symbol GADD45B, optionally in combination with one or more genes labeled as 4.300 to 4.488 identified in Table 4.

266) In another aspect the invention employs one or more genes according to any one of paragraphs 1-265, wherein the gene has the symbol FLJ10847, optionally in combination with one or more genes labeled as 4.301 to 4.488 identified in Table 4.

267) In another aspect the invention employs one or more genes according to any one of paragraphs 1-266, wherein the gene has the symbol KIF21B, optionally in combination with one or more genes labeled as 4.302 to 4.488 identified in Table 4.

268) In another aspect the invention employs one or more genes according to any one of paragraphs 1-267, wherein the gene has the symbol CCND2, optionally in combination with one or more genes labeled as 4.303 to 4.488 identified in Table 4.

269) In another aspect the invention employs one or more genes according to any one of paragraphs 1-268, wherein the gene has the symbol PRG1, optionally in combination with one or more genes labeled as 4.304 to 4.488 identified in Table 4.

270) In another aspect the invention employs one or more genes according to any one of paragraphs 1-269, wherein the gene has the symbol SLC40A1, optionally in combination with one or more genes labeled as 4.307 to 4.488 identified in Table 4.

271) In another aspect the invention employs one or more genes according to any one of paragraphs 1-270, wherein the gene has the symbol CRIP1, optionally in combination with one or more genes labeled as 4.308 to 4.488 identified in Table 4.

272) In another aspect the invention employs one or more genes according to any one of paragraphs 1-271, wherein the gene has the symbol LOC283070, optionally in combination with one or more genes labeled as 4.309 to 4.488 identified in Table 4.

273) In another aspect the invention employs one or more genes according to any one of paragraphs 1-272, wherein the gene has the symbol SIGLEC1, optionally in combination with one or more genes labeled as 4.310 to 4.488 identified in Table 4.

274) In another aspect the invention employs one or more genes according to any one of paragraphs 1-273, wherein the gene has the symbol ZNF11B, optionally in combination with one or more genes labeled as 4.311 to 4.488 identified in Table 4.

275) In another aspect the invention employs one or more genes according to any one of paragraphs 1-274, wherein the gene has the symbol CXCR4, optionally in combination with one or more genes labeled as 4.312 to 4.488 identified in Table 4.

276) In another aspect the invention employs one or more genes according to any one of paragraphs 1-275, wherein the gene has the symbol HLA-DMA, optionally in combination with one or more genes labeled as 4.313 to 4.488 identified in Table 4

277) In another aspect the invention employs one or more genes according to any one of paragraphs 1-276, wherein the gene has the symbol MRC1, optionally in combination with one or more genes labeled as 4.315 to 4.488 identified in Table 4.

278) In another aspect the invention employs one or more genes according to any one of paragraphs 1-277, wherein the gene has the symbol LMO2, optionally in combination with one or more genes labeled as 4.315a to 4.488 identified in Table 4.

279) In another aspect the invention employs one or more genes according to any one of paragraphs 1-278, wherein the gene has the symbol DENND2D, optionally in combination with one or more genes labeled as 4.316 to 4.488 identified in Table 4.

280) In another aspect the invention employs one or more genes according to any one of paragraphs 1-279, wherein the gene has the symbol CCL18, optionally in combination with one or more genes labeled as 4.317 to 4.488 identified in Table 4.

281) In another aspect the invention employs one or more genes according to any one of paragraphs 1-280, wherein the gene has the symbol P2RY13, optionally in combination with one or more genes labeled as 4.319 to 4.488 identified in Table 4.

282) In another aspect the invention employs one or more genes according to any one of paragraphs 1-281, wherein the gene has the symbol ANGPTL1, optionally in combination with one or more genes labeled as 4.320 to 4.488 identified in Table 4.

283) In another aspect the invention employs one or more genes according to any one of paragraphs 1-282, wherein the gene is identifiable by probe set number 230391_at, optionally in combination with one or more genes labeled as 4.322 to 4.488 identified in Table 4.

284) In another aspect the invention employs one or more genes according to any one of paragraphs 1-283, wherein the gene has the symbol C8orf51, optionally in combination with one or more genes labeled as 4.323 to 4.488 identified in Table 4.

285) In another aspect the invention employs one or more genes according to any one of paragraphs 1-284, wherein the gene has the symbol GIMAP8, optionally in combination with one or more genes labeled as 4.324 to 4.488 identified in Table 4.

286) In another aspect the invention employs one or more genes according to any one of paragraphs 1-285, wherein the gene is identifiable by probe set number 2277880_at, optionally in combination with one or more genes labeled as 4.325 to 4.488 identified in Table 4.

287) In another aspect the invention employs one or more genes according to any one of paragraphs 1-286, wherein the gene has the symbol JAK2, optionally in combination with one or more genes labeled as 4.326 to 4.488 identified in Table 4.

288) In another aspect the invention employs one or more genes according to any one of paragraphs 1-287, wherein the gene has the symbol TNFSF10, optionally in combination with one or more genes labeled as 4.327 to 4.488 identified in Table 4.

289) In another aspect the invention employs one or more genes according to any one of paragraphs 1-288, wherein the gene has the symbol C1R, optionally in combination with one or more genes labeled as 4.328 to 4.488 identified in Table 4.

290) In another aspect the invention employs one or more genes according to any one of paragraphs 1-289, wherein the gene has the symbol ACPL2, optionally in combination with one or more genes labeled as 4.329 to 4.488 identified in Table 4.

291) In another aspect the invention employs one or more genes according to any one of paragraphs 1-290, wherein the gene has the symbol TNFRSF19, optionally in combination with one or more genes labeled as 4.331 to 4.488 identified in Table 4.

292) In another aspect the invention employs one or more genes according to any one of paragraphs 1-291, wherein the gene has the symbol LRP12, optionally in combination with one or more genes labeled as 4.332 to 4.488 identified in Table 4.

293) In another aspect the invention employs one or more genes according to any one of paragraphs 1-292, wherein the gene is identifiable by probe set number 1557116_at, optionally in combination with one or more genes labeled as 4.334 to 4.488 identified in Table 4.

294) In another aspect the invention employs one or more genes according to any one of paragraphs 1-293, wherein the gene has the symbol PRKCB1, optionally in combination with one or more genes labeled as 4.335 to 4.488 identified in Table 4.

295) In another aspect the invention employs one or more genes according to any one of paragraphs 1-294, wherein the gene has the symbol IPO11, optionally in combination with one or more genes labeled as 4.336 to 4.488 identified in Table 4.

296) In another aspect the invention employs one or more genes according to any one of paragraphs 1-295, wherein the gene has the symbol DLGAP1, optionally in combination with one or more genes labeled as 4.337 to 4.488 identified in Table 4.

297) In another aspect the invention employs one or more genes according to any one of paragraphs 1-296, wherein the gene has the symbol PRKAR2B, optionally in combination with one or more genes labeled as 4.338 to 4.488 identified in Table 4.

298) In another aspect the invention employs one or more genes according to any one of paragraphs 1-297, wherein the gene has the symbol MAP3K8, optionally in combination with one or more genes labeled as 4.339 to 4.488 identified in Table 4.

299) In another aspect the invention employs one or more genes according to any one of paragraphs 1-298, wherein the gene has the symbol EVI2B, optionally in combination with one or more genes labeled as 4.340 to 4.488 identified in Table 4.

300) In another aspect the invention employs one or more genes according to any one of paragraphs 1-299, wherein the gene has the symbol GBP1, optionally in combination with one or more genes labeled as 4.341 to 4.488 identified in Table 4.

301) In another aspect the invention employs one or more genes according to any one of paragraphs 1-300, wherein the gene has the symbol CXCL10, optionally in combination with one or more genes labeled as 4.342 to 4.488 identified in Table 4.

302) In another aspect the invention employs one or more genes according to any one of paragraphs 1-301, wherein the gene has the symbol CAMK2N1, optionally in combination with one or more genes labeled as 4.343 to 4.488 identified in Table 4

303) In another aspect the invention employs one or more genes according to any one of paragraphs 1-302, wherein the gene has the symbol MED12L, optionally in combination with one or more genes labeled as 4.344 to 4.488 identified in Table 4.

304) In another aspect the invention employs one or more genes according to any one of paragraphs 1-303, wherein the gene has the symbol ID2, optionally in combination with one or more genes labeled as 4.345 to 4.488 identified in Table 4.

305) In another aspect the invention employs one or more genes according to any one of paragraphs 1-304, wherein the gene has the symbol CTBP2, optionally in combination with one or more genes labeled as 4.346 to 4.488 identified in Table 4.

306) In another aspect the invention employs one or more genes according to any one of paragraphs 1-305, wherein the gene has the symbol IGLJ3, optionally in combination with one or more genes labeled as 4.347 to 4.488 identified in Table 4.

307) In another aspect the invention employs one or more genes according to any one of paragraphs 1-306, wherein the gene has the symbol GBP4, optionally in combination with one or more genes labeled as 4.348 to 4.488 identified in Table 4.

308) In another aspect the invention employs one or more genes according to any one of paragraphs 1-307, wherein the gene has the symbol LOC439949, optionally in combination with one or more genes labeled as 4.349 to 4.488 identified in Table 4.

309) In another aspect the invention employs one or more genes according to any one of paragraphs 1-308, wherein the gene has the symbol FBXO16, optionally in combination with one or more genes labeled as 4.350 to 4.488 identified in Table 4.

310) In another aspect the invention employs one or more genes according to any one of paragraphs 1-309, wherein the gene has the symbol PRF1, optionally in combination with one or more genes labeled as 4.351 to 4.488 identified in Table 4.

311) In another aspect the invention employs one or more genes according to any one of paragraphs 1-310, wherein the gene has the symbol TRAM2, optionally in combination with one or more genes labeled as 4.352 to 4.488 identified in Table 4.

312) In another aspect the invention employs one or more genes according to any one of paragraphs 1-311, wherein the gene has the symbol LYN, optionally in combination with one or more genes labeled as 4.353 to 4.488 identified in Table 4.

313) In another aspect the invention employs one or more genes according to any one of paragraphs 1-312, wherein the gene has the symbol CENTD1, optionally in combination with one or more genes labeled as 4.355 to 4.488 identified in Table 4.

314) In another aspect the invention employs one or more genes according to any one of paragraphs 1-313, wherein the gene has the symbol FLJ20273, optionally in combination with one or more genes labeled as 4.356 to 4.488 identified in Table 4.

315) In another aspect the invention employs one or more genes according to any one of paragraphs 1-314, wherein the gene has the symbol TFEC, optionally in combination with one or more genes labeled as 4.357 to 4.488 identified in Table 4.

316) In another aspect the invention employs one or more genes according to any one of paragraphs 1-315, wherein the gene has the symbol PPP1R16B, optionally in combination with one or more genes labeled as 4.358 to 4.488 identified in Table 4.

317) In another aspect the invention employs one or more genes according to any one of paragraphs 1-316, wherein the gene has the symbol CD48, optionally in combination with one or more genes labeled as 4.359 to 4.488 identified in Table 4.

318) In another aspect the invention employs one or more genes according to any one of paragraphs 1-317, wherein the gene has the symbol HLA-DPB1, optionally in combination with one or more genes labeled as 4.361 to 4.488 identified in Table 4.

319) In another aspect the invention employs one or more genes according to any one of paragraphs 1-318, wherein the gene has the symbol GTPBP5, optionally in combination with one or more genes labeled as 4.362 to 4.488 identified in Table 4.

320) In another aspect the invention employs one or more genes according to any one of paragraphs 1-319, wherein the gene has the symbol GBP5, optionally in combination with one or more genes labeled as 4.363 to 4.488 identified in Table 4.

321) In another aspect the invention employs one or more genes according to any one of paragraphs 1-320, wherein the gene has the symbol MAP B, optionally in combination with one or more genes labeled as 4.364 to 4.488 identified in Table 4.

322) In another aspect the invention employs one or more genes according to any one of paragraphs 1-321, wherein the gene has the symbol EXTL3, optionally in combination with one or more genes labeled as 4.365 to 4.488 identified in Table 4.

323) In another aspect the invention employs one or more genes according to any one of paragraphs 1-322, wherein the gene has the symbol CORO1A, optionally in combination with one or more genes labeled as 4.366 to 4.488 identified in Table 4.

324) In another aspect the invention employs one or more genes according to any one of paragraphs 1-323, wherein the gene has the symbol PDGFRL, optionally in combination with one or more genes labeled as 4.367 to 4.488 identified in Table 4.

325) In another aspect the invention employs one or more genes according to any one of paragraphs 1-324, wherein the gene has the symbol RP9, optionally in combination with one or more genes labeled as 4.368 to 4.488 identified in Table 4.

326) In another aspect the invention employs one or more genes according to any one of paragraphs 1-325, wherein the gene has the symbol RHOU, optionally in combination with one or more genes labeled as 4.369 to 4.488 identified in Table 4.

327) In another aspect the invention employs one or more genes according to any one of paragraphs 1-326, wherein the gene has the symbol MTAC2D1, optionally in combination with one or more genes labeled as 4.370 to 4.488 identified in Table 4.

328) In another aspect the invention employs one or more genes according to any one of paragraphs 1-327, wherein the gene has the symbol CCL8, optionally in combination with one or more genes labeled as 4.371 to 4.488 identified in Table 4.

329) In another aspect the invention employs one or more genes according to any one of paragraphs 1-328, wherein the gene has the symbol CECR1, optionally in combination with one or more genes labeled as 4.373 to 4.488 identified in Table 4.

330) In another aspect the invention employs one or more genes according to any one of paragraphs 1-329, wherein the gene has the symbol SLC40A1, optionally in combination with one or more genes labeled as 4.374 to 4.488 identified in Table 4.

331) In another aspect the invention employs one or more genes according to any one of paragraphs 1-330, wherein the gene has the symbol ADCY6, optionally in combination with one or more genes labeled as 4.375 to 4.488 identified in Table 4.

332) In another aspect the invention employs one or more genes according to any one of paragraphs 1-331, wherein the gene has the symbol CP, optionally in combination with one or more genes labeled as 4.376 to 4.488 identified in Table 4.

333) In another aspect the invention employs one or more genes according to any one of paragraphs 1-332, wherein the gene has the symbol EDG1, optionally in combination with one or more genes labeled as 4.377 to 4.488 identified in Table 4.

334) In another aspect the invention employs one or more genes according to any one of paragraphs 1-333, wherein the gene has the symbol RGS3, optionally in combination with one or more genes labeled as 4.379 to 4.488 identified in Table 4.

335) In another aspect the invention employs one or more genes according to any one of paragraphs 1-334, wherein the gene is identifiable by probe set number 228339_at, optionally in combination with one or more genes labeled as 4.380 to 4.488 identified in Table 4.

336) In another aspect the invention employs one or more genes according to any one of paragraphs 1-335, wherein the gene has the symbol ABHD5, optionally in combination with one or more genes labeled as 4.381 to 4.488 identified in Table 4.

337) In another aspect the invention employs one or more genes according to any one of paragraphs 1-336, wherein the gene has the symbol MS4A7, optionally in combination with one or more genes labeled as 4.382 to 4.488 identified in Table 4.

338) In another aspect the invention employs one or more genes according to any one of paragraphs 1-337, wherein the gene has the symbol PRKCH, optionally in combination with one or more genes labeled as 4.384 to 4.488 identified in Table 4.

339) In another aspect the invention employs one or more genes according to any one of paragraphs 1-338, wherein the gene has the symbol LOC286071, optionally in combination with one or more genes labeled as 4.385 to 4.488 identified in Table 4.

340) In another aspect the invention employs one or more genes according to any one of paragraphs 1-339, wherein the gene has the symbol BLNK, optionally in combination with one or more genes labeled as 4.386 to 4.488 identified in Table 4.

341) In another aspect the invention employs one or more genes according to any one of paragraphs 1-340, wherein the gene is identifiable by the probe set number 242546 at, optionally in combination with one or more genes labeled as 4.387 to 4.488 identified in Table 4.

342) In another aspect the invention employs one or more genes according to any one of paragraphs 1-341, wherein the gene has the symbol PCDHGC3, optionally in combination with one or more genes labeled as 4.390 to 4.488 identified in Table 4.

343) In another aspect the invention employs one or more genes according to any one of paragraphs 1-342, wherein the gene has the symbol CAMSAP1L1, optionally in combination with one or more genes labeled as 4.391 to 4.488 identified in Table 4.

344) In another aspect the invention employs one or more genes according to any one of paragraphs 1-343, wherein the gene has the symbol NPY1R, optionally in combination with one or more genes labeled as 4.392 to 4.488 identified in Table 4.

345) In another aspect the invention employs one or more genes according to any one of paragraphs 1-344, wherein the gene has the symbol CD274, optionally in combination with one or more genes labeled as 4.393 to 4.488 identified in Table 4.

346) In another aspect the invention employs one or more genes according to any one of paragraphs 1-345, wherein the gene has the symbol PGM5, optionally in combination with one or more genes labeled as 4.394 to 4.488 identified in Table 4.

347) In another aspect the invention employs one or more genes according to any one of paragraphs 1-346, wherein the gene has the symbol PLCG2, optionally in combination with one or more genes labeled as 4.395 to 4.488 identified in Table 4.

348) In another aspect the invention employs one or more genes according to any one of paragraphs 1-347, wherein the gene has the symbol TNFSF10, optionally in combination with one or more genes labeled as 4.397 to 4.488 identified in Table 4.

349) In another aspect the invention employs one or more genes according to any one of paragraphs 1-348, wherein the gene has the symbol BTG2, optionally in combination with one or more genes labeled as 4.398 to 4.488 identified in Table 4.

350) In another aspect the invention employs one or more genes according to any one of paragraphs 1-349, wherein the gene has the symbol LAMP3, optionally in combination with one or more genes labeled as 4.399 to 4.488 identified in Table 4.

351) In another aspect the invention employs one or more genes according to any one of paragraphs 1-350, wherein the gene has the symbol IGLC1, optionally in combination with one or more genes labeled as 4.400 to 4.488 identified in Table 4.

352) In another aspect the invention employs one or more genes according to any one of paragraphs 1-351, wherein the gene has the symbol SIPA1L1, optionally in combination with one or more genes labeled as 4.401 to 4.488 identified in Table 4.

353) In another aspect the invention employs one or more genes according to any one of paragraphs 1-352 wherein the gene has the symbol AIF1, optionally in combination with one or more genes labeled as 4.402 to 4.488 identified in Table 4.

354) In another aspect the invention employs one or more genes according to any one of paragraphs 1-353, wherein the gene has the symbol IGLC2, optionally in combination with one or more genes labeled as 4.403 to 4.488 identified in Table 4.

355) In another aspect the invention employs one or more genes according to any one of paragraphs 1-354, wherein the gene has the symbol B2M, optionally in combination with one or more genes labeled as 4.404 to 4.488 identified in Table 4.

356) In another aspect the invention employs one or more genes according to any one of paragraphs 1-355, wherein the gene has the symbol CLEC7A, optionally in combination with one or more genes labeled as 4.405 to 4.488 identified in Table 4.

357) In another aspect the invention employs one or more genes according to any one of paragraphs 1-356, wherein the gene has the symbol MGC17330, optionally in combination with one or more genes labeled as 4.406 to 4.488 identified in Table 4.

358) In another aspect the invention employs one or more genes according to any one of paragraphs 1-357, wherein the gene has the symbol IGF1R, optionally in combination with one or more genes labeled as 4.407 to 4.488 identified in Table 4.

359) In another aspect the invention employs one or more genes according to any one of paragraphs 1-358, wherein the gene has the symbol HIVEP1, optionally in combination with one or more genes labeled as 4.408 to 4.488 identified in Table 4.

360) In another aspect the invention employs one or more genes according to any one of paragraphs 1-359, wherein the gene has the symbol FKBP14, optionally in combination with one or more genes labeled as 4.409 to 4.488 identified in Table 4.

361) In another aspect the invention employs one or more genes according to any one of paragraphs 1-360, wherein the gene has the symbol LAPTM5, optionally in combination with one or more genes labeled as 4.410 to 4.488 identified in Table 4.

362) In another aspect the invention employs one or more genes according to any one of paragraphs 1-361, wherein the gene has the symbol AB13BP, optionally in combination with one or more genes labeled as 4.411 to 4.488 identified in Table 4.

363) In another aspect the invention employs one or more genes according to any one of paragraphs 1-362, wherein the gene has the symbol HLA-E, optionally in combination with one or more genes labeled as 4.412 to 4.488 identified in Table 4.

364) In another aspect the invention employs one or more genes according to any one of paragraphs 1-363, wherein the gene has the symbol ARLAC, optionally in combination with one or more genes labeled as 4.413 to 4.488 identified in Table 4.

365) In another aspect the invention employs one or more genes according to any one of paragraphs 1-364, wherein the gene has the symbol ASS, optionally in combination with one or more genes labeled as 4.415 to 4.488 identified in Table 4.

366) In another aspect the invention employs one or more genes according to any one of paragraphs 1-365, wherein the gene has the symbol ITGB3, optionally in combination with one or more genes labeled as 4.417 to 4.488 identified in Table 4.

367) In another aspect the invention employs one or more genes according to any one of paragraphs 1-366, wherein the gene has the symbol SYK, optionally in combination with one or more genes labeled as 4.417 to 4.488 identified in Table 4.

368) In another aspect the invention employs one or more genes according to any one of paragraphs 1-366, wherein the gene has the symbol RAC2, optionally in combination with one or more genes labeled as 4.418 to 4.488 identified in Table 4.

369) In another aspect the invention employs one or more genes according to any one of paragraphs 1-368, wherein the gene is identifiable by probe set number 1557222_at, optionally in combination with one or more genes labeled as 4.419 to 4.488 identified in Table 4.

370) In another aspect the invention employs one or more genes according to any one of paragraphs 1-369, wherein the gene has the symbol CD3G, optionally in combination with one or more genes labeled as 4.420 to 4.488 identified in Table 4.

371) In another aspect the invention employs one or more genes according to any one of paragraphs 1-370, wherein the gene has the symbol IGF1, optionally in combination with one or more genes labeled as 4.421 to 4.488 identified in Table 4.

372) In another aspect the invention employs one or more genes according to any one of paragraphs 1-371, wherein the gene is identifiable by probe set number 228858_at, optionally in combination with one or more genes labeled as 4.422 to 4.488 identified in Table 4.

373) In another aspect the invention employs one or more genes according to any one of paragraphs 1-372, wherein the gene is has the symbol CYB5A, optionally in combination with one or more genes labeled as 4.423 to 4.488 identified in Table 4.

374) In another aspect the invention employs one or more genes according to any one of paragraphs 1-373, wherein the gene is has the symbol TTC25, optionally in combination with one or more genes labeled as 4.424 to 4.488 identified in Table 4.

375) In another aspect the invention employs one or more genes according to any one of paragraphs 1-374, wherein the gene is has the symbol SLAMF6, optionally in combination with one or more genes labeled as 4.425 to 4.488 identified in Table 4.

376) In another aspect the invention employs one or more genes according to any one of paragraphs 1-375, wherein the gene is has the symbol ARHGAP21, optionally in combination with one or more genes labeled as 4.426 to 4.488 identified in Table 4.

377) In another aspect the invention employs one or more genes according to any one of paragraphs 1-376, wherein the gene is has the symbol FLOT1, optionally in combination with one or more genes labeled as 4.428 to 4.488 identified in Table 4.

378) In another aspect the invention employs one or more genes according to any one of paragraphs 1-377, wherein the gene is has the symbol IBRDC2 optionally in combination with one or more genes labeled as 4.429 to 4.488 identified in Table 4.

379) In another aspect the invention employs one or more genes according to any one of paragraphs 1-378, wherein the gene is has the symbol KIAA1794, optionally in combination with one or more genes labeled as 4.430 to 4.488 identified in Table 4.

380) In another aspect the invention employs one or more genes according to any one of paragraphs 1-379, wherein the gene is has the symbol OLFML1, optionally in combination with one or more genes labeled as 4.431 to 4.488 identified in Table 4.

381) In another aspect the invention employs one or more genes according to any one of paragraphs 1-380, wherein the gene is has the symbol GMFG, optionally in combination with one or more genes labeled as 4.432 to 4.488 identified in Table 4.

382) In another aspect the invention employs one or more genes according to any one of paragraphs 1-381, wherein the gene is has the symbol TNFRSF1B, optionally in combination with one or more genes labeled as 4.433 to 4.488 identified in Table 4.

383) In another aspect the invention employs one or more genes according to any one of paragraphs 1-382, wherein the gene is identifiable by probe set number 217629_at, optionally in combination with one or more genes labeled as 4.434 to 4.488 identified in Table 4.

384) In another aspect the invention employs one or more genes according to any one of paragraphs 1-383, wherein the gene is has the symbol DEF6, optionally in combination with one or more genes labeled as 4.436 to 4.488 identified in Table 4.

385) In another aspect the invention employs one or more genes according to any one of paragraphs 1-384, wherein the gene is has the symbol MAP4K4, optionally in combination with one or more genes labeled as 4.437 to 4.488 identified in Table 4.

386) In another aspect the invention employs one or more genes according to any one of paragraphs 1-385, wherein the gene is has the symbol CMKOR1, optionally in combination with one or more genes labeled as 4.438 to 4.488 identified in Table 4.

387) In another aspect the invention employs one or more genes according to any one of paragraphs 1-386, wherein the gene is identifiable by probe set number 1563461_at, optionally in combination with one or more genes labeled as 4.439 to 4.488 identified in Table 4.

388) In another aspect the invention employs one or more genes according to any one of paragraphs 1-387, wherein the gene is has the symbol CHKA, optionally in combination with one or more genes labeled as 4.440 to 4.488 identified in Table 4.

389) In another aspect the invention employs one or more genes according to any one of paragraphs 1-388, wherein the gene is identifiable by probe set number 226865_at, optionally in combination with one or more genes labeled as 4.441 to 4.488 identified in Table 4.

390) In another aspect the invention employs one or more genes according to any one of paragraphs 1-389, wherein the gene has the symbol HS3ST3B1, optionally in combination with one or more genes labeled as 4.442 to 4.488 identified in Table 4.

391) In another aspect the invention employs one or more genes according to any one of paragraphs 1-390, wherein the gene has the symbol CXorf9, optionally in combination with one or more genes labeled as 4.443 to 4.488 identified in Table 4.

392) In another aspect the invention employs one or more genes according to any one of paragraphs 1-391, wherein the gene has the symbol EVI2A, optionally in combination with one or more genes labeled as 4.445 to 4.488 identified in Table 4.

393) In another aspect the invention employs one or more genes according to any one of paragraphs 1-392, wherein the gene has the symbol NFAM1, optionally in combination with one or more genes labeled as 4.446 to 4.488 identified in Table 4.

394) In another aspect the invention employs one or more genes according to any one of paragraphs 1-393, wherein the gene is identifiable by probe set number 242874_at, optionally in combination with one or more genes labeled as 4.447 to 4.488 identified in Table 4.

395) In another aspect the invention employs one or more genes according to any one of paragraphs 1-394, wherein the gene has the symbol ATP5J, optionally in combination with one or more genes labeled as 4.450 to 4.488 identified in Table 4.

396) In another aspect the invention employs one or more genes according to any one of paragraphs 1-395, wherein the gene has the symbol CYLD, optionally in combination with one or more genes labeled as 4.451 to 4.488 identified in Table 4.

397) In another aspect the invention employs one or more genes according to any one of paragraphs 1-396, wherein the gene has the symbol GIMAP6, optionally in combination with one or more genes labeled as 4.452 to 4.488 identified in Table 4.

398) In another aspect the invention employs one or more genes according to any one of paragraphs 1-397, wherein the gene has the symbol MFAP4, optionally in combination with one or more genes labeled as 4.453 to 4.488 identified in Table 4.

399) In another aspect the invention employs one or more genes according to any one of paragraphs 1-398, wherein the gene has the symbol TUBB2B, optionally in combination with one or more genes labeled as 4.454 to 4.488 identified in Table 4.

400) In another aspect the invention employs one or more genes according to any one of paragraphs 1-399, wherein the gene has the symbol NELL2, optionally in combination with one or more genes labeled as 4.455 to 4.488 identified in Table 4.

401) In another aspect the invention employs one or more genes according to any one of paragraphs 1-400, wherein the gene is identifiable by probe set number 236583_at, optionally in combination with one or more genes labeled as 4.456 to 4.488 identified in Table 4.

402) In another aspect the invention employs one or more genes according to any one of paragraphs 1-401, wherein the gene has the symbol IL1RN, optionally in combination with one or more genes labeled as 4.457 to 4.488 identified in Table 4.

403) In another aspect the invention employs one or more genes according to any one of paragraphs 1-402, wherein the gene has the symbol KIAA1211, optionally in combination with one or more genes labeled as 4.459 to 4.488 identified in Table 4.

404) In another aspect the invention employs one or more genes according to any one of paragraphs 1-403, wherein the gene has the symbol ADAMDEC1, optionally in combination with one or more genes labeled as 4.460 to 4.488 identified in Table 4.

405) In another aspect the invention employs one or more genes according to any one of paragraphs 1-404, wherein the gene has the symbol AOC3, optionally in combination with one or more genes labeled as 4.461 to 4.488 identified in Table 4.

406) In another aspect the invention employs one or more genes according to any one of paragraphs 1-405, wherein the gene has the symbol SAMHD1, optionally in combination with one or more genes labeled as 4.463 to 4.488 identified in Table 4.

407) In another aspect the invention employs one or more genes according to any one of paragraphs 1-406, wherein the gene has the symbol SLC22A3, optionally in combination with one or more genes labeled as 4.465 to 4.488 identified in Table 4.

408) In another aspect the invention employs one or more genes according to any one of paragraphs 1-407, wherein the gene has the symbol IGLV3-25, optionally in combination with one or more genes labeled as 4.466 to 4.488 identified in Table 4.

409) In another aspect the invention employs one or more genes according to any one of paragraphs 1-408, wherein the gene is identifiable by probe set number 1556185_a_at, optionally in combination with one or more genes labeled as 4.467 to 4.488 identified in Table 4.

410) In another aspect the invention employs one or more genes according to any one of paragraphs 1-409, wherein the gene has the symbol RAB11FIP1, optionally in combination with one or more genes labeled as 4.468- to 4.488 identified in Table 4.

411) In another aspect the invention employs one or more genes according to any one of paragraphs 1-410, wherein the gene has the symbol PER2, optionally in combination with one or more genes labeled as 4.469 to 4.488 identified in Table 4.

412) In another aspect the invention employs one or more genes according to any one of paragraphs 1-411, wherein the gene has the symbol TTL, optionally in combination with one or more genes labeled as 4.470 to 4.488 identified in Table 4.

413) In another aspect the invention employs one or more genes according to any one of paragraphs 1-412, wherein the gene has the symbol SIAHBP1, optionally in combination with one or more genes labeled as 4.472 to 4.488 identified in Table 4.

414) In another aspect the invention employs one or more genes according to any one of paragraphs 1-413, wherein the gene has the symbol FLJ22536, optionally in combination with one or more genes labeled as 4.473 to 4.488 identified in Table 4.

415) In another aspect the invention employs one or more genes according to any one of paragraphs 1-414, wherein the gene has the symbol RP6-213H19.1, optionally in combination with one or more genes labeled as 4.474 to 4.488 identified in Table 4.

416) In another aspect the invention employs one or more genes according to any one of paragraphs 1-415, wherein the gene is identifiable by probe set number 235804_at, optionally in combination with one or more genes labeled as 4.475 to 4.488 identified in Table 4.

417) In another aspect the invention employs one or more genes according to any one of paragraphs 1-416, wherein the gene has the symbol NCF4, optionally in combination with one or more genes labeled as 4.476 to 4.488 identified in Table 4.

418) In another aspect the invention employs one or more genes according to any one of paragraphs 1-417, wherein the gene has the symbol EPSTI1, optionally in combination with one or more genes labeled as 4.477 to 4.488 identified in Table 4.

419) In another aspect the invention employs one or more genes according to any one of paragraphs 1-418, wherein the gene has the symbol LOC441212, optionally in combination with one or more genes labeled as 4.478 to 4.488 identified in Table 4.

420) In another aspect the invention employs one or more genes according to any one of paragraphs 1-419, wherein the gene has the symbol ANK3, optionally in combination with one or more genes labeled as 4.479 to 4.488 identified in Table 4.

421) In another aspect the invention employs one or more genes according to any one of paragraphs 1-420, wherein the gene has the symbol PCDH9, optionally in combination with one or more genes labeled as 4.480 to 4.488 identified in Table 4.

422) In another aspect the invention employs one or more genes according to any one of paragraphs 1-421, wherein the gene has the symbol C21orf86, optionally in combination with one or more genes labeled as 4.481 to 4.488 identified in Table 4.

423) In another aspect the invention employs one or more genes according to any one of paragraphs 1-422, wherein the gene has the symbol DHRS9, optionally in combination with one or more genes labeled as 4.482 to 4.488 identified in Table 4.

424) In another aspect the invention employs one or more genes according to any one of paragraphs 1-423, wherein the gene has the symbol ARHGAP25, optionally in combination with one or more genes labeled as 4.483 to 4.488 identified in Table 4.

425) In another aspect the invention employs one or more genes according to any one of paragraphs 1-424, wherein the gene has the symbol TRAF4, optionally in combination with one or more genes labeled as 4.484 to 4.488 identified in Table 4.

426) In another aspect the invention employs one or more genes according to any one of paragraphs 1-425, wherein the gene has the symbol LST1, optionally in combination with one or more genes labeled as 4.485 to 4.488 identified in Table 4.

427) In another aspect the invention employs one or more genes according to any one of paragraphs 1-426, wherein the gene has the symbol PALMD, optionally in combination with one or more genes labeled as 4.486 to 4.488 identified in Table 4.

428) In another aspect the invention employs one or more genes according to any one of paragraphs 1-427, wherein the gene has the symbol TAP1, optionally in combination with one or more genes labeled as 4.487 to 4.488 identified in Table 4.

429) In another aspect the invention employs one or more genes according to any one of paragraphs 1-428, wherein the gene has the symbol MSX2, optionally in combination with one or more genes labeled as 4.448 identified in Table 4.

430) In another aspect the invention employs one or more genes according to any one of paragraphs 1-429, wherein the gene has the symbol SIRPG.

In one aspect the invention employs a gene listed in Table 7.

TABLE 7
Gene Symbol

CCL5
FASLG
GNLY
GZMB
PRF1
IFNG
ICOS
TBX21
CD8A
CD3E
CXCL11
CXCL10
CXCR3
CD20
FOXP3
INDO
CD45Ro
CD45R
CD69
TRBV19
TRAT1
TRDV2
STAT4
PRKCQ
GMZK
GPR171
UBD
CD52
CD3D
IL7R
IRF1
TLR7

In one aspect the invention employs one or more genes selected from Table 9.

TABLE 9
Gene Symbol

PRF1
IRF1
GZMB
GNLY
CD8A
PRKCQ
FOXP3
IFNG
CCL5
GPR171
TRBV19
CD3E
TBX21
FASLG
CXCL10
ICOS
CXCR3
CXCL11

In one aspect the invention employs one or more genes selected from Table 11.

TABLE 11

| Gene Symbol | Gene Title |
|---|---|
| CCL5 | chemokine (C-C motif) ligand 5 |
| UCHL1 | Ubiquitin carboxyl-terminal esterase L1 (ubiquitin thiolesterase) |
| PVT1 /// LOC441378 | Pvt1 oncogene homolog, MYC activator (mouse) /// LOC441378 |
| — | CDNA clone IMAGE: 4796388 |
| CD52 | CD52 antigen (CAMPATH-1 antigen) /// CD52 antigen (CAMPATH-1 |
| | antigen) |
| UBD | ubiquitin D |
| STAT4 | signal transducer and activator of transcription 4 |
| GZMK | granzyme K (granzyme 3; tryptase II) /// granzyme K (granzyme 3; tryptase |
| | II) |
| IL18RAP | interleukin 18 receptor accessory protein |
| GPR171 | G protein-coupled receptor 171 |
| PSCDBP | pleckstrin homology, Sec7 and coiled-coil domains, binding protein /// |
| | pleckstrin homology, Sec7 and coiled-coil domains, binding protein |
| NCF2 | neutrophil cytosolic factor 2 (65 kDa, chronic granulomatous disease, |
| | autosomal 2) |
| PRKCQ | protein kinase C, theta |
| LST1 | leukocyte specific transcript 1 |
| TRA@ /// TRDV2 /// | T cell receptor alpha locus /// T cell receptor delta variable 2 /// T cell receptor |
| TRAV20 /// TRAJ17 | alpha variable 20 /// T cell receptor alpha joining 17 /// T cell receptor alpha |
| /// TRAC | constant |
| TRGC2 | T cell receptor gamma constant 2 |
| TRBV21-1 /// | T cell receptor beta variable 21-1 /// T cell receptor beta variable 19 /// T cell |
| TRBV19 /// TRBV5-4 | receptor beta variable 5-4 /// T cell receptor beta variable 3-1 /// T cell receptor |
| /// TRBV3-1 /// | beta constant 1 |
| TRBC1 | |
| HLA-DQA1 /// HLA- | major histocompatibility complex, class II, DQ alpha 1 /// major |
| DQA2 | histocompatibility complex, class II, DQ alpha 2 |
| TRBV19 /// TRBC1 | T cell receptor beta variable 19 /// T cell receptor beta variable 19 /// T cell |
| | receptor beta constant 1 /// T cell receptor beta constant 1 |
| CD3D | CD3D antigen, delta polypeptide (TiT3 complex) |
| GADD45B | Growth arrest and DNA-damage-inducible, beta |
| ITGB3 | integrin, beta 3 (platelet glycoprotein IIIa, antigen CD61) |
| TRAT1 | T cell receptor associated transmembrane adaptor 1 |
| HLA-DMA | major histocompatibility complex, class II, DM alpha |
| CENTD3 | centaurin, delta 3 |
| SAMSN1 | SAM domain, SH3 domain and nuclear localisation signals, 1 |
| CLEC4E | C-type lectin domain family 4, member E |
| TNFRSF19 | tumor necrosis factor receptor superfamily, member 19 |
| NAV1 | neuron navigator 1 |
| LOXL4 | lysyl oxidase-like 4 |
| TNFRSF19 | tumor necrosis factor receptor superfamily, member 19 |
| TAGAP | T-cell activation GTPase activating protein |
| GIMAP2 | GTPase, IMAP family member 2 |
| IGF1R | Insulin-like growth factor 1 receptor |
| C17orf63 | Chromosome 17 open reading frame 63 |
| CD52 | CD52 antigen (CAMPATH-1 antigen) |

In one aspect the invention employs one or more genes selected from Table 12.

TABLE 12

| Gene symbol | Gene title |
|---|---|
| CCL5 | chemokine (C-C motif) ligand 5 |
| TRAT1 | T cell receptor associated transmembrane adaptor 1 |
| STAT4 | signal transducer and activator of transcription 4 |
| PRKCQ | protein kinase C, theta |
| GPR171 | G protein-coupled receptor 171 |
| UBD | ubiquitin D |
| CD52 | CD52 molecule |
| CD3D | CD3d molecule, delta (CD3-TCR complex) |
| PRF1 | perforin 1 (pore forming protein) |
| CD8A | CD8a molecule |
| CXCL10 | chemokine (C—X—C motif) ligand 10 |
| CD69 | CD69 molecule |
| TRBV19 | T cell receptor beta variable 19 |
| TRDV2 | T cell receptor delta variable 2 |
| IL7R | interleukin 7 receptor |
| GZMK | granzyme K |
| CD45R | PROTEIN-TYROSINE PHOSPHATASE, RECEPTOR- |
| | TYPE, C (PTPRC) |

In one aspect the invention provides one or more genes selected from Table 13.

TABLE 13

| Gene | |
|---|---|
| symbol | Gene title |
| FASLG | Fas ligand (TNF superfamily, member 6) |
| GNLY | granulysin |
| GZMB | granzyme B (granzyme 2, cytotoxic T-lymphocyte-associated |
| | serine esterase 1) |
| IFNG | interferon, gamma |
| ICOS | inducible T-cell co-stimulator |
| TBX21 | T-box 21 |
| CD3E | CD3e molecule, epsilon (CD3-TCR complex) |
| CXCL11 | chemokine (C—X—C motif) ligand 11 |
| CXCR3 | chemokine (C—X—C motif) receptor 3 |
| CD20 | CD20 molecule |
| FOXP3 | forkhead box P3 |
| INDO | indoleamine-pyrrole 2,3 dioxygenase |
| IRF1 | interferon regulatory factor 1 |
| TLR7 | toll-like receptor 7 |

PCR is a more sensitive technique than microarray and therefore can detect lower levels of differentially expressed genes.

In particular the following genes are suitable for PCR analysis: IL7R, CD3D, CD3E, CD52, UBD, GPR171, GMZK, PRKCQ, STAT4, TRDV2, TRAT1, TRBV19, CD69, INDO, CD45R, CD45RO, FOXP3, CD20, CCL5, FASLG, GNLY, GZMB, PRF1, IFNG, ICOS, TBX21, CD8A, CD3E, CXCL10, CXCL11, IRF1, TLR7 and CXCR3.

In one aspect the gene(s) employed are selected from the group comprising or consisting of: CCL5, TRAT1, STAT4, PRKCQ, GPR171, UBD, CD52, CD3D, PRF1, CD8A, CXCL10, CD69, TRBV19, TRDV2, IL7R, GZMK and CD45R.

In one aspect the gene(s) employed are selected from the comprising or consisting of: FASLG, GNLY, GZMB, IFNG, ICOS, TBX21, CD3E, CXCL11, CXCR3, CD20, FOXP3, INDO, IRF1 and TLR7.

The gene(s) FOXP3 and/or PRF1 is/are particularly suitable for PCR analysis according to the invention.

Suitable targets for immunohistochemistry include CD3, CD8, CD86, LAMP, CD20, CD45RO, CXCR3, CXL10/11, CD69, granzyme B, IDO, B cells and gene products from one or more genes from the NK family, HLA family, T cell receptor family and/or activated T cell.

In a further aspect of the invention there is provided a gene signature indicative of a non-responder, for example wherein one or more of immune genes such as those listed herein are NOT differentially expressed and/or are down regulated and/or are NOT upregulated.

In one aspect the gene is NOT FOXP3.

According to a further aspect the present invention provides a gene signature indicative of an increased likelihood of a patients responding favourably to appropriate immunotherapy, which in turn is likely to result in an improved survival of patients, for example with Mage expressing cancers following treatment with Mage specific immunotherapy. This gene signature, of at least 5, 6, 7, 8, 9, 10, 11, 12, 13, 14, 15, 16, 17, 18, 19, 20 21, 22, 23, 24, 25, 26, 27, 28, 29, 30 or 31 genes from the genes disclosed in Table 1, is characterised by differential expression compared to the gene signature of Mage-expressing tumour patients who do not respond to Mage antigen specific cancer immunotherapy.

In one aspect the invention provides a profile based on differential expression of 1 or more of 62 known genes that relate to immune infiltration and activation.

The predictive genes correspond mainly to expression of and often upregulation of genes related to immune infiltration and activation. These genes include HLA class II, Interleukin-2 receptor gamma, T cell receptor genes (TRBV19, TRAT1, TRGC2), granzyme, and CD69.

In one embodiment there is provided a gene signature, present in patients, for example with or who have had MAGE-expressing tumours, who respond to treatment, in which one or more, for example at least 5, suitably 6, 7, 8, 9, 10 of the genes of Table 2 are differentially expressed:

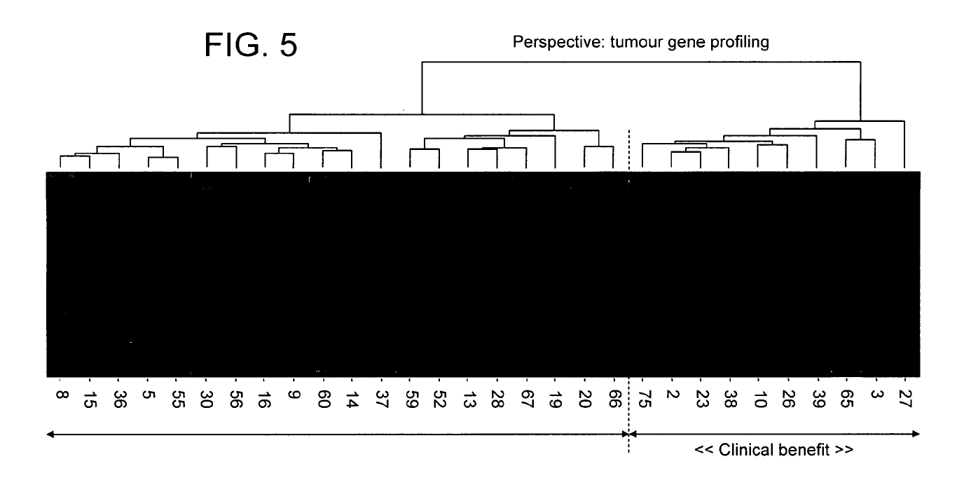

As shown in FIG. 5a below, characterisation of responders and non-responders may be based on the differential expression on only one or two genes, such as TCR, CD3 and/or IL-7. Other genes that are thought to be particularly suitable in methods employing only one or two genes include: IL7R, CD3D, CD3E, CD52, UBD, GPR171, GMZK, PRKCQ, STAT4, TRDV2, TRAT1, TRBV19, CD69, INDO, CD45R, CD45RO, FOXP3, CD20, CCL5, FASLG, GNLY, GZMB, PRF1, IFNG, ICOS, TBX21, CD8A, CD3E, CXCL10, CXCL11, TRF1, TLR7 and CXCR3, which may require the use of appropriately sensitive analytical techniques.

The invention herein extends to use of all permutations of the genes listed herein for identification of said signature/profile.

The invention also extends to embodiments according to the invention described herein, which comprise, consist essentially of, or consists of the components/elements described.

The invention extends to the functional equivalents of genes listed herein, for example as characterised by hierarchical classification of genes such as described by Hongwei Wu et al 2007 (Hierarchical classification of equivalent genes in prokaryotes-Nucliec Acid Research Advance Access).

Whilst not wishing to be bound by theory, it is thought that is not necessarily the gene per se that is characteristic of the signature but rather it is the gene function which is fundamentally important. Thus a functionally equivalent gene to an immune activation gene such as those listed above, for example in Table 1, 2, 3, 4, 7, 9, 11, 12 or 13 may be employed in the signature, see for example, Journal of the National Cancer Institute Vol 98, No. 7 Apr. 5, 2006.

The genes were identified by specific probes and thus a skilled person will understand that the description of the genes above is a description based on current understanding of what hybridises to the probe. However, regardless of the nomenclature used for the genes by repeating the hybridisation to the relevant probe under the prescribed conditions the requisite gene can be identified.

The invention extends to use of the profile(s) according to the invention for predicting or identifying a patient as a responder or non-responder to immunotherapy, such as cancer immunotherapy, for example cancer testis immunotherapy.

Thus the invention includes a method of analyzing a patient derived sample, based on differential expression of the profile/gene(s) according to the invention for the purpose of characterising the patient from which the sample was derived as a responder or non-responder to immunotherapy. In one aspect the invention provides a method of identifying a profile according to the invention comprising the steps:
a) analyzing a patient derived sample for differential expression of one or more immune response genes, and
b) characterising the patient from which the sample was derived as a responder or non-responder to appropriate immunotherapy, based on the results of step (a), wherein the characterisation step is optionally performed by reference or comparison to a standard.

Suitable standards are described above.

The present invention therefore, generally relates, in one aspect, to a method for the detection of a gene signature in a biological sample, the method comprising the analysis of the expression of one or more genes of Tables 1, 2, 3, 4, 7, 9, 11, 12 or 13, for example at least 5, 6, 7, 8, 9, 10, 11, 12, 13, 14, 15, 16, 17, 18, 19, 2021, 22, 23, 24, 25, 26, 27, 28, 29, 30 or 31 genes (eg for genes set forth in Table 1). In an embodiment, the analysis comprises the expression of at least 5, suitably 6, 7, 8, 9 or 10 genes set forth in Table 2.

In one aspect the invention provides a method for measuring expression levels of polynucleotides from immune activation genes such as one or more genes listed in Tables 1, 2, 3, 4, 7, 9, 11, 12 and/or 13 in a sample for the purpose of identifying if the patient, from whom the sample was derived, is likely to be a responder or non-responder to immunotherapy such a cancer immunotherapy comprising the steps:
isolating the RNA from the sample,
optionally amplifying the copies of the cDNA from the sample for said genes, and
quantifying the levels of cDNA in the sample.

In one embodiment, the diagnostic method comprises determining whether a subject expresses any of the gene products of the genes set forth in Table 1 or any other embodiment of the invention described herein by, for example, detecting the level of the corresponding mRNA and/or protein level of the gene products of the genes set forth in Table 1 etc. For example, by using techniques such as by Northern blot analysis, reverse transcription-polymerase chain reaction (RT-PCR), in situ hybridization, immunoprecipitation, Western blot hybridization, or immunohistochemistry. According to the method, cells may be obtained from a subject and the levels of the protein, or mRNA, of the analyzed genes, compared to that of a non-responder patient. Patients who differentially express one or more, for example, 5, 6, 7, 8, 9, 10, 1, 12, 13, 14, 15, 16, 17, 18, 19, 20 21, 22, 23, 24, 25, 26, 27, 28, 29, 30 or 31 genes selected from Table 1 or another embodiment of the invention are those which can be predicted to benefit from immunotherapy, for example cancer immunotherapy, such as Mage antigen specific cancer immunotherapy. For example, a nucleic acid molecule which hybridizes to a given location on a microarray is said to be differentially expressed if the hybridization signal is, for example, higher than the hybridization signal at the same location on an identical array hybridized with a nucleic acid sample obtained from a subject that does not clinically respond to Mage specific immunotherapy.

The results of 30 patients who have been subjected to gene profiling are shown in FIG. 5. This pictorially demonstrates that the gene signature of the present invention is aligned with clinical response to MAGE-antigen specific cancer immunotherapy.

Alternatively the cancer patient may be characterised as a responder or non-responder to immunotherapy from a visual inspection of a tissue section derived from the cancer or tumour. A responder is likely to be a patient with an infiltration of immune response cells into the cancer/tumour microenvironment.

The invention also provides a method of generating a new gene profile not specifically recited herein, based on differential expression of one or more immune response/activation genes, for indicating whether a patient is likely to be a responder or non-responder to appropriate immunotherapy, for example cancer immunotherapy such as Mage immunotherapy comprising the steps:
a) analysing at least two patient derived samples for differential expression of one or more of immune activation genes, where the sample group comprises both responders and non-responders to appropriate immunotherapy, and
b) correlating same with clinical outcome, after appropriate treatment, of patients from which the samples were derived, and
c) identifying one or more genes which are differentially expressed in responders and/O non-responders.

After the new profile has been identified the invention also extends to analysis of a previously uncharacterised sample, designating same as responders or non-responders (as appropriate) and if desired administering a therapeutically effective amount of an appropriate immunotherapy, for example cancer immunotherapy such as Mage immunotherapy to one or more patients designated as responders.

The invention also provides a method of generating a gene profile based on differential expression of one or more gene sequences recited in Table 1, 2, 3, 4, 7, 9, 11, 12 and/or 13 for indicating whether a patient is likely to be a responder or non-responder to immunotherapy comprising the steps:
a) analyzing at least two patient derived samples for differential expression of
   (i) one or more gene sequences recited in Table 1, 2, 3, 4, 7, 9, 11, 12 and/or 13 and
   (ii) optionally one or more gene sequences not recited in Table 1, 2, 3, 4, 7, 9, 11, 12 and/or 13,
where the sample group comprises both responders and non-responders to appropriate immunotherapy;
b) correlating differential expression of said one or more gene sequences with clinical outcome, after appropriate treatment, of patients from which the samples were derived, and
c) identifying one or more genes which are differentially expressed in responders and non-responders.

After the profile has been identified the invention also extends to analysis of a previously uncharacterised sample, designating same as responders or non-responders (as appropriate) and if desired administering a therapeutically effective amount of an appropriate immunotherapy, for example cancer immunotherapy such as Mage immunotherapy to one or more patients designated as responders.

The invention provides a diagnostic kit comprising at least one component for performing an analysis on a patient derived sample to identify a profile according to the invention, the results of which may be used to designate a patient from which the sample was derived as a responder or non-responder to immunotherapy.

The kit may comprise materials/reagents for PCR (such as QPCR), microarray analysis, immunohistochemistry or other analytical technique that may be used for accessing differential expression of one or more genes.

The invention also provides a diagnostic kit comprising a set of probes capable of hybridising to the mRNA or cDNA of one or more, such as at least 5 genes as set forth in Table 1 or alternatively Tables 2, 3, 4, 7, 9, 11, 12 and/or 13, for example a diagnostic kit comprising a set of probes capable of hybridising to the mRNA or its cDNA of at least 6, 7, 8, 9, 10, 11, 12, 13, 14, 15, 16, 17, 18, 19, 20, 21, 22, 23, 24, 25, 26, 27, 28, 29, 30 or 31 genes as set forth in Table 1.

In another embodiment this invention relates to diagnostic kits. For example, diagnostic kits containing such microarrays comprising a microarray substrate and probes that are capable of hybridising to mRNA or cDNA expressed from, for example, 5, 6, 7, 8, 9, 10, 11, 12, 13, 14, 15, 16, 17, 18, 19, 20 21, 22, 23, 24, 25, 26, 27, 28, 29, 30 or 31 genes from, for example Table 1 or any other embodiment of the invention that are capable of demonstrating the gene signature of the invention.

In one aspect the invention provides microarrays adapted for identification of a signature according to the invention.

The invention also extends to substrates and probes suitable for hybridising to an mRNA or cDNA moiety expressed from one or more genes employed in the invention, for example from Table 1, 2, 3, 4, 7, 9, 11, 12 or 13.

Commercially available microarrays contain many more probes than are required to characterise the differential expression of the genes under consideration at any one time, to aid the accuracy of the analysis. Thus one or more probe sets may recognise the same gene.

Thus in one embodiment multiple probes or probe sets are used to identify if an immune activation gene is differentially expressed, such as upregulated.

The diagnostic kit may, for example comprise probes, which are arrayed in a microarray. Specifically, prepared microarrays, for example, containing one or more probe sets described herein can readily be prepared by companies such as Affymetrix, thereby providing a specific test and optionally reagents for identifying the profile, according to the invention.

In an embodiment the microarrays or diagnostic kits will additionally be able to test for the presence or absence of the relevant cancer testis antigen expressing gene such as the Mage gene. Thus in one aspect the invention provides a probe and/or probe set suitable for said hybridisation, under appropriate conditions. The invention also extends to use of probes, for example as described herein or functional equivalents thereof, for the identification of a gene profile according to the present invention.

The invention herein described extends to use of all permutations of the probes listed herein (or functional analogues thereof) for identification of the said signature.

In one aspect the invention provides use of a probe for the identification of differential expression of at least one gene product of an immune activation gene for establishing if a gene profile according to the present invention is present in a patient derived sample.

Table 1B discloses probe sets from which probes of typically 25 mer in length may be designed and which are suitable for identifying the mRNA (or its cDNA) expressed from, for example the genes of Table 1 (or alternatively Table 2, 3, 4, 7, 9, 11, 12 or 13). Such probes and probe sets are an aspect of the invention. Typically each probe set may comprise about or exactly 11 individual sequences of about or exactly 25 nucleotides, which correspond precisely to a run of sequences from the probe set.

Accordingly, in one aspect, this invention relates to oligonucleotide probes and primers capable of recognising the mRNA (or its cDNA) expressed from the genes from Table 1, (or alternatively Table 2, 3, 4, 7, 9, 11, 12 or 13) and diagnostic kits based on these probes and primers. Such kits may include probes or kits for the detection of a Mage gene.

In an aspect the invention provides a profile based on the differential expression of one or more of the genes of Table 3 identifiable by one or more of following 13 probes: 204661_at, 205890_at, 206118_at, 206666_at, 207651_at, 210038_at, 210972_x_at, 211144_x_at, 211796_s_at, 213193_x_at, 213539_at, 217147_s_at, 34210_at.

Further details of these probes and the target genes of the same are given in Table 3A below. Hybridisation will generally be preformed under stringent conditions, such as 3×SSC, 0.1% SDS, at 50° C.

Once the target gene(s)/profile has/have been identified then it is well within the skilled person's ability to design alternative probes that hybridise to the same target. Therefore the invention also extends to probes, which under appropriate conditions measure the same differential expression of the gene(s) of the present invention to provide a signature/profile as described.

The invention also extends to use of the relevant probe in analysis of whether a cancer patient will be a responder or non-responder to treatment with an appropriate immunotherapy.

The invention also extends to use (and processes employing same) of known microarrays for identification of said signature.

A nucleic acid probe may be at least 10, 15, 20, 25, 30, 35, 40, 50, 75, 100 or more nucleotides in length and may comprise the full length gene. Probes for use in the invention are those that are able to hybridise specifically to the mRNA (or its cDNA) expressed from the genes listed in Table 1 (or Table 2, 3, 4, 7, 9, 11, 12 or 13) under stringent conditions.

The present invention further relates to a method of screening the effects of a drug on a tissue or cell sample comprising the step of analysing the expression profile of, for example 5, 6, 7, 8, 9, 10, 11, 12, 13, 14, 15, 16, 17, 18, 19, 20 21, 22, 23, 24, 25, 26, 27, 28, 29, 30 or 31 genes selected from Table 1 or any other embodiment of the invention described herein before and after drug treatment. The invention therefore provides a method for screening for a drug, which would alter the gene profile to that of a patient having improved survival following treatment with, for example, Mage antigen specific cancer immunotherapy (ie. to alter the gene profile to that of a responder), to enable the patient to benefit from, for example, Mage antigen specific cancer immunotherapy.

The present invention further provides a method of patient diagnosis comprising, for example, the step of analysing the expression profile of, for example, 5, 6, 7, 8, 9, 10, 11, 12, 13, 14, 15, 16, 17, 18, 19, 20 21, 22, 23, 24, 25, 26, 27, 28, 29, 30 or 31 genes selected from Table 1 or any other embodiment of the invention described herein and comparing it with a standard to diagnose whether the patient would benefit from Mage specific immunotherapy.

The invention includes a method of patient diagnosis comprising the step of analysing the expression profile of at least 5, 6, 7, 8, 9, 10, 11, 12, 13, 14, 15, 16, 17, 18, 19, 20 21, 22, 23, 24, 25, 26, 27, 28, 29, 30 or 31 genes selected from Table 1 or other embodiment of the invention from a tumour tissue sample given by a patient and assessing whether 5 or more of said genes are expressed.

Thus in clinical applications, tissue samples from a human patient may be screened for the presence and/or absence of the expression of, for example 5, 6, 7, 8, 9, 10, 11, 12, 13, 14, 15, 16, 17, 18, 19, 20 21, 22, 23, 24, 25, 26, 27, 28, 29, 30 or 31 genes selected from Table 1 or any other embodiment of the invention described herein.

In the context of the present invention, the sample may be of any biological tissue or fluid derived from a patient potentially in need of treatment. The sample maybe derived from sputum, blood, urine, or from solid tissues such as biopsy from a primary tumour or metastasis, or from sections of previously removed tissues.

Samples could comprise or consist of, for example, needle biopsy cores, surgical resection samples or lymph node tissue. These methods include obtaining a biopsy, which is optionally fractionated by cryostat sectioning to enrich tumour cells to about 80% of the total cell population. In certain embodiments, nucleic acids extracted from these samples may be amplified using techniques well known in the art. The levels of selected markers (eg the gene products of table 1) can be detected and can be compared with statistically valid groups of, for example, Mage positive non responder patients.

For cancer, the biological sample will contain cancer or tumour cells and may, for example, be derived from the cancer or tumour such as a fresh sample (including frozen samples) or a sample that has been preserved in paraffin. Having said this, samples preserved in paraffin can suffer from degradation and the profile observed may be modified. A person working the in field is well able to compensate of these changes observed by recalibrating the parameters of the profile.

### Microarrays

A microarray is an array of discrete regions, typically nucleic acids, which are separate from one another and are typically arrayed at a density of between, about 100/cm² to 1000/cm², but can be arrayed at greater densities such as 10000/cm². The principle of a microarray experiment, is that mRNA from a given cell line or tissue is used to generate a labeled sample typically labeled cDNA, termed the ‘target’, which is hybridized in parallel to a large number of, nucleic acid sequences, typically DNA sequences, immobilised on a solid surface in an ordered array. Tens of thousands of transcript species can be detected and quantified simultaneously. Although many different microarray systems have been developed the most commonly used systems today can be divided into two groups, according to the arrayed material: complementary DNA (cDNA) and oligonucleotide microarrays. The arrayed material has generally been termed the probe since it is equivalent to the probe used in a northern blot analysis. Probes for cDNA arrays are usually products of the polymerase chain reaction (PCR) generated from cDNA libraries or clone collections, using either vector-specific or gene-specific primers, and are printed onto glass slides or nylon membranes as spots at defined locations. Spots are typically 10-300 μm in size and are spaced about the same distance apart. Using this technique, arrays consisting of more than 30,000 cDNAs can be fitted onto the surface of a conventional microscope slide. For oligonucleotide arrays, short 20-25mers are synthesized in situ, either by photolithography onto silicon wafers (high-density-oligonucleotide arrays from Affymetrix or by ink-jet technology (developed by Rosetta Inpharmatics, and licensed to Agilent Technologies). Alternatively, presynthesized oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of presynthesized longer oligonucleotides (50-100mers) has recently been developed to counteract these disadvantages. Thus in performing a microarray to ascertain whether a patient presents with a gene signature of the present invention, the following steps are performed: obtain mRNA from the sample and prepare nucleic acids targets, contact the array under conditions, typically as suggested by the manufactures of the microarray (suitably stringent hybridisation conditions such as 3×SSC, 0.1% SDS, at 50° C.) to bind corresponding probes on the array, wash if necessary to remove unbound nucleic acid targets and analyse the results.

It will be appreciated that the mRNA may be enriched for sequences of interest such as those in Table 1 or 2 (or other embodiment of the invention) by methods known in the art, such as primer specific cDNA synthesis. The population may be further amplified, for example, by using PCR technology. The targets or probes are labeled to permit detection of the hybridisation of the target molecule to the microarray. Suitable labels include isotopic or fluorescent labels which can be incorporated into the probe.

In an alternative embodiment, a patient may be diagnosed to ascertain whether his/her tumor expresses the gene signature of the invention utilising a diagnostic kit based on PCR technology, in particular Quantative PCR (For a review see Ginzinger D Experimental haematology 30 (2002) p 503-512 and Giuliette et al Methods, 25 p 386 (2001).

In an alternative aspect the invention provides a method further comprising the steps of analyzing a tumour derived sample to determine which antigen(s) are expressed by the tumour and hence enabling administration of an a therapeutically effective amount of an appropriate antigen specific cancer immunotherapeutic, for example where the tumour is found to be MAGE (such as Mage A3) positive, appropriate treatment may, for example, include administration of Mage A3 antigen specific immunotherapy.

A sample such as tumour tissue of a patient is deemed to present the gene signature of the invention if one or more genes of Table 1, 2, 3, 4, 7, 9, 11, 12 and/or 13 are differentially expressed (such as upregulated), for example, 5, 6, 7, 8, 9, 10, 11, 12, 13, 14, 15, 16, 17, 18, 19, 20 21, 22, 23, 24, 25, 26, 27, 28, 29, 30 or 31 genes from for example Table 1 or other embodiment of the invention are expressed and can be detected by microarray analysis or other appropriate analysis for example as described herein. The tumour tissue preferably shows expression of at least 5 genes selected from Table 1, 2, 3, 4, 7, 9, 11, 12 and/or 13, more preferably 10 genes, particularly 5, 6, 7, 8, 9 or 10 genes from Table 1, 2, 3, 4, 7, 9, 11, 12 or 13.

### Immunotherapeutics

In a further aspect the invention provides a method of treating a responder patient with an appropriate immunotherapy, for example cancer immunotherapy such as cancer testis immunotherapy, after identification of the same as a responder thereto.

Thus the invention provides a method of treating a patient comprising the step of administering a therapeutically effective amount of an appropriate immunotherapy (for example cancer immunotherapy, such as Mage cancer immunotherapy), after first characterising the patient as a responder based on differential expression of at least one immune activation gene, for example as shown by appropriate analysis of a sample derived from the patient. In particular wherein the patient is characterised as a responder based on one or more embodiments described herein.

In one aspect the immunotherapy comprises an appropriate adjuvant (immunostimulant), see description below.

In yet a further embodiment of the invention there is provided a method of treating a patient suffering from, for example, a Mage expressing tumour, the method comprising determining whether the patient expresses the gene signature of the invention and then administering, for example, a Mage specific immunotherapeutic. In a further embodiment, the patient is treated with, for example, the Mage specific immunotherapy to prevent or ameliorate recurrence of disease, after first receiving treatment such as resection by surgery of any tumour or other chemotherapeutic or radiotherapy treatment.

A further aspect of the invention is a method of treating a patient suffering from a Mage expressing tumour, the method comprising determining whether the patient's tumour expresses at least 5, 6, 7, 8, 9, 10, 11, 12, 13, 14, 15, 16, 17, 18, 19, 2021, 22, 23, 24, 25, 26, 27, 28, 29, 30 or 31 genes selected from Table 1 (or other embodiment of the invention) from a tumour tissue sample given by a patient and then administering a Mage specific immunotherapeutic to said patient.

Also provided is a method of treating a patient susceptible to recurrence of Mage expressing tumour having been treated to remove/treat a Mage expressing tumour, the method comprising determining whether the patient's tumour expresses at least 5, 6, 7, 8, 9, 10, 11, 12, 13, 14, 15, 16, 17, 18, 19, 20 21, 22, 23, 24, 25, 26, 27, 28, 29, 30 or 31 genes selected from Table 1 (or other embodiment of the invention) from a tumour tissue sample given by a patient and then administering a Mage specific immunotherapeutic.

The invention also provides as method of treatment or use employing:
MAGE specific immunotherapeutic comprising a MAGE antigen or peptide thereof,
MAGE antigen comprising a MAGE-A3 protein or peptide,
MAGE antigen comprising the peptide EVDPIGHLY,
MAGE antigen or peptide fused or conjugated to a carrier protein, for example in which the carrier protein is selected from protein D, NS1 or CLytA or fragments thereof, and/or
MAGE specific immunotherapeutic further comprises an adjuvant, for example in which the adjuvant comprises one or more or combinations of: 3D-MPL; aluminium salts; CpG containing oligonucleotides; saponin-containing adjuvants such as QS21 or ISCOMs; oil-in-water emulsions; and liposomes.

The invention also extends to use of an immunotherapy such as a cancer immunotherapy, in particular Mage immunotherapy in the manufacture of a medicament for the treatment of a patient such as a cancer patient designated as a responder, thereto.

It was observed that one patient initially characterised as a non-responder was subsequently characterised as responder after radiation therapy. Interestingly the inventors also believe that it may be possible to induce a responders profile in at least some non-responders, for example by subjecting the patient to radiation therapy, or administering an inflammatory stimulant such as interferon (for example imiquimod such as administered topically) or a TLR3 (for example as described in WO 2006/054177), 4, 7, 8 or TLR 9 agonist (for example containing a CpG motif, in particular administering a high dose thereof such as 0.1 to 75 mg per Kg adminstered, for example weekly). See for example Krieg, A. M., Efler, S. M., Wittpoth, M., Al Adhami, M. J. & Davis, H. L. Induction of systemic TH 1-like innate immunity in normal volunteers following subcutaneous but not intravenous administration of CPG 7909, a synthetic B-class CpG oligodeoxynucleotide TLR9 agonist. J. Immunother. 27, 460-471 (2004).

The high dose of CpG may, for example be inhaled or given subcutaneously.

Whilst not wishing to be bound by theory, it is hypothesised that the radiation therapy stimulated a systemic inflammatory response/immune response, which once triggered rendered the patient (or tumour tissue therein) more responsive to immunotherapy.

Accordingly, the invention further provides a method of inducing a responders profile by stimulating a systemic immune response, for example by administering an immunostimulant. The invention further provides the use of Mage specific immunotherapy in the manufacture of a medicament for the treatment of patients suffering from Mage expressing tumour or patients who have received treatment (e.g. surgery, chemotherapy or radiotherapy) to remove/treat a Mage expressing tumour, said patient expressing the gene signature of the invention.

The immunotherapy may then be administered once the responders profile has been induced. In one aspect the invention provides use of Mage specific immunotherapy in the manufacture of a medicament for the treatment of patients suffering from Mage expressing tumour, said patient characterised by their tumour expressing at least 5, 6, 7, 8, 9, 10, 11, 12, 13, 14, 15, 16, 17, 18, 19, 20 21, 22, 23, 24, 25, 26, 27, 28, 29, 30 or 31 genes selected from Table 1, or alternatively other embodiments of the invention.

The invention also provides use of Mage specific immunotherapy in the manufacture of a medicament for the treatment of patients susceptible to recurrence from Mage expressing tumour said patient characterised by their tumour expressing at least 5, 6, 7, 8, 9, 10, 11, 12, 13, 14, 15, 16, 17, 18, 19, 2021, 22, 23, 24, 25, 26, 27, 28, 29, 30 or 31 genes selected from Table 1 or alternatively other embodiments of the invention.

Advantageously, the invention may allow medics to target those populations of patients that will obtain a clinical benefit from receiving an appropriate immunotherapy. It is expected that after screening that at least 60% of patients such as 70, 75, 80, 85% or more of patients deemed/characterised as responders will receive a clinical benefit from the immunotherapy, which is a significant increase over the current levels observed with therapy such as cancer therapy generally.

Advantageously if the cancer immunotherapy is given concomitantly or subsequent to chemotherapy it may assist in raising the patient's immune responses, which may have been depleted by the chemotherapy.

Antigen Specific Cancer Immunotherapeutics (ASCIs) suitable for use in the invention may, for example include those capable of raising a Mage specific immune response. Such immunotherapeutics may be capable of raising an immune response to a Mage gene product, for example a Mage-A antigen such as Mage-A3. The immunotherapeutic will generally contain at least one epitope from a Mage gene product. Such an epitope may be present as a peptide antigen optionally linked covalently to a carrier and optionally in the presence of an adjuvant. Alternatively larger protein fragments may be used. For example, the immunotherapeutic for use in the invention may comprise an antigen that corresponds to or comprises amino acids 195-279 of MAGE-A1. The fragments and peptides for use must however, when suitably presented be capable of raising a Mage specific immune response. Examples of peptides that may be used in the present invention include the MAGE-3.A1 nonapeptide EVDPIGHLY [Seq. ID No 36] (see Marchand et al., International Journal of Cancer 80(2), 219-230), and the following MAGE-A3 peptides:

| | |
|---|---|
| FLWGPRALV; | [Seq. ID No 37] |
| MEVDPIGHLY; | [Seq. ID No 38] |
| VHFLLLKYRA; | [Seq. ID No 39] |
| LVHFLLLKYR; | [Seq. ID No 40] |
| LKYRAREPVT; | [Seq. ID No 41] |
| ACYEFLWGPRALVETS; | [Seq. ID No 42] |
| and | |
| TQHFVQENYLEY; | [Seq. ID No 43] |

Alternative ASCIs include cancer testis antigens such as PRAME, LAGE 1, LAGE 2, and others, for example details of which can be obtained from www.cancerimmunity.org/CTdatabase. The cancer immunotherapy may be based, for example on one or more of the antigens discussed below.

In one embodiment of the present invention, the antigen to be used may consist or comprise a MAGE tumour antigen, for example, MAGE 1, MAGE 2, MAGE 3, MAGE 4, MAGE 5, MAGE 6, MAGE 7, MAGE 8, MAGE 9, MAGE 10, MAGE 11 or MAGE 12. The genes encoding these MAGE antigens are located on chromosome X and share with each other 64 to 85% homology in their coding sequence (De Plaen, 1994). These antigens are sometimes known as MAGE A1, MAGE A2, MAGE A3, MAGE A4, MAGE A5, MAGE A6, MAGE A7, MAGE A8, MAGE A9, MAGE A 10, MAGE A11 and/or MAGE A12 (The MAGE A family). In one embodiment, the antigen is MAGE A3.

In one embodiment, an antigen from one of two further MAGE families may be used: the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the Mage C family currently includes MAGE C1 and MAGE C2.

In general terms, a MAGE protein can be defined as containing a core sequence signature located towards the C-terminal end of the protein (for example with respect to MAGE A1 a 309 amino acid protein, the core signature corresponds to amino acid 195-279).

The consensus pattern of the core signature is thus described as follows wherein x represents any amino acid, lower case residues are conserved (conservative variants allowed) and upper case residues are perfectly conserved.

### Core Sequence Signature

LixvL(2×)I(3×)g(2×)apEExiWexl(2×)m(3-4×)Gxe(3-4×)gxp(2×)llt(3×)VqexYLxYxqVPxsxP(2×)yeFLWGprA(2×)Et(3×)kv
   Conservative substitutions are well known and are generally set up as the default scoring matrices in sequence alignment computer programs. These programs include PAM250 (Dayhoft M. O. et al., (1978), “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft and Joija G. Henikoft (1992), “Amino acid substitution matricies from protein blocks”), Proc. Natl. Acad. Sci. USA 89 (Biochemistry): 10915-10919.

In general terms, substitution within the following groups are conservative substitutions, but substitutions between groups are considered non-conserved. The groups are:
i) Aspartate/asparagine/glutamate/glutaminc
ii) Serine/threonine
iii) Lysine/arginine
iv) Phenylalanine/tyrosine/tryptophane
v) Leucine/isoleucine/valine/methionine
vi) Glycine/alanine

In general and in the context of this invention, a MAGE protein will be approximately 50% or more identical, such as 70, 80, 90, 95 or 99% identical, in this core region with amino acids 195 to 279 of MAGE A1.

MAGE protein derivatives are also known in the art, see: WO 99/40188. Such derivatives are suitable for use in therapeutic vaccine formulations (Immunotherapeutic) which are suitable for the treatment of a range of tumour types.

Several CTL epitopes have been identified on the MAGE-3 protein. One such epitope, MAGE-3.A1, is a nonapeptide sequence located between amino acids 168 and 176 of the MAGE-3 protein which constitutes an epitope specific for CTLs when presented in association with the MHC class I molecule HLA.A1. Recently two additional CTL epitopes have been identified on the peptide sequence of the MAGE-3 protein by their ability to mount a CTL response in a mixed culture of melanoma cells and autologous lymphocytes. These two epitopes have specific binding motifs for the HLA.A2 (Van der Bruggen, 1994) and HLA.B44 (Herman, 1996) alleles respectively.

In a further embodiment of the invention, the tumour antigen may comprise or consist of one of the following antigens, or an immunogenic portion thereof which is able to direct an immune response to the antigen:

SSX-2; SSX-4; SSX-5; NA17; MELAN-A; Tyrosinase; LAGE-1; NY-ESO-1; PRAME; P790; P510; P835; B305D; B854; CASB618 (as described in WO00/53748); CASB7439 (as described in WO01/62778); C1491; C1584; and C1585.

In one embodiment, the antigen may comprise or consist of P501S (also known as prostein). The P501S antigen may be a recombinant protein that combines most of the P501S protein with a bacterial fusion protein comprising the C terminal part of protein LytA of Streptococcus pneumoniae in which the P2 universal T helper peptide of tetanus toxoid has been inserted, ie. a fusion comprising CLytA-P2-CLyta (the “CPC” fusion partner), as described in WO03/104272;

In one embodiment, the antigen may comprise or consist of WT-1 expressed by the Wilm's tumor gene, or its N-terminal fragment WT-1F comprising about or approximately amino acids 1-249; the antigen expressed by the Her-2/neu gene, or a fragment thereof. In one embodiment, the Her-2/neu antigen may be one of the following fusion proteins which are described in WO00/44899.

In a further embodiment, the antigen may comprise or consist of “HER-2/neu ECD-ICD fusion protein,” also referred to as “ECD-ICD” or “ECD-ICD fusion protein,” which refers to a fusion protein (or fragments thereof) comprising the extracellular domain (or fragments thereof) and the intracellular domain (or fragments thereof) of the HER-2/neu protein. In one embodiment, this ECD-ICD fusion protein does not include a substantial portion of the HER-2/neu transmembrane domain, or does not include any of the HER-2/neu transmembrane domain.

In a further embodiment, the antigen may comprise or consist of “HER-2/neu ECD-PD fusion protein,” also referred to as “ECD-PD” or “ECD-PD fusion protein,” or the “HER-2/neu ECD-ΔPD fusion protein,” also referred to as “ECD-ΔPD” or “ECD-ΔPD fusion protein,” which refers to fusion proteins (or fragments thereof) comprising the extracellular domain (or fragments thereof) and phosphorylation domain (or fragments thereof, e.g., ΔPD) of the HER-2/neu protein.

In one embodiment, the ECD-PD and ECD-ΔPD fusion proteins do not include a substantial portion of the HER-2/neu transmembrane domain, or does not include any of the HER-2/neu transmembrane domain.

In one embodiment, the antigen may comprise a Mage or other appropriate protein linked to an immunological fusion or expression enhancer partner. Fusion proteins may include a hybrid protein comprising two or more antigens relevant to a given disease or may be a hybrid of an antigen and an expression enhancer partner.

The antigen and partner may be chemically conjugated, or may be expressed as a recombinant fusion protein. In an embodiment in which the antigen and partner are expressed as a recombinant fusion protein, this may allow increased levels to be produced in an expression system compared to non-fused protein. Thus the fusion partner may assist in providing T helper epitopes (immunological fusion partner), preferably T helper epitopes recognised by humans, and/or assist in expressing the protein (expression enhancer) at higher yields than the native recombinant protein. In one embodiment, the fusion partner may be both an immunological fusion partner and expression enhancing partner.

In one embodiment of the invention, the immunological fusion partner that may be used is derived from protein D, a surface protein of the gram-negative bacterium, Haemophilus influenza B (WO 91/18926) or a derivative thereof. The protein D derivative may comprise the first ⅓ of the protein, or approximately or about the first ⅓ of the protein, in particular it may comprise the first N-terminal 100-110 amino acids or approximately the first N-terminal 100-110 amino acids.

In one embodiment the fusion protein comprises the first 109 residues (or 108 residues therefrom) or amino acids 20 to 127 of protein D.

Other fusion partners that may be used include the non-structural protein from influenzae virus, NS1 (hemagglutinin). Typically the N terminal 81 amino acids of NS1 may be utilised, although different fragments may be used provided they include T-helper epitopes.

In another embodiment the immunological fusion partner is the protein known as LytA. LytA is derived from Streptococcus pneumoniae which synthesise an N-acetyl-L-alanine amidase, amidase LytA, (coded by the LytA gene (Gene, 43 (1986) page 265-272) an autolysin that specifically degrades certain bonds in the peptidoglycan backbone. The C-terminal domain of the LytA protein is responsible for the affinity to the choline or to some choline analogues such as DEAE. This property has been exploited for the development of E. coli C-LytA expressing plasmids useful for expression of fusion proteins. Purification of hybrid proteins containing the C-LytA fragment at its amino terminus has been described (Biotechnology: 10, (1992) page 795-798). In one embodiment, the C terminal portion of the molecule may be used. The embodiment may utilise the repeat portion of the LytA molecule found in the C terminal end starting at residue 178. In one embodiment, the LytA portion may incorporate residues 188-305.

In one embodiment of the present invention, the Mage protein may comprise a derivatised free thiol. Such antigens have been described in WO 99/40188. In particular carboxyamidated or carboxymethylated derivatives may be used.

In one embodiment of the present invention, the tumour associated antigen comprises a Mage-A3-protein D molecule. The nucleotide and amino acid sequences for this molecule are shown in seq ID No 35. This antigen and those summarised below are described in more detail in WO 99/40188.

In further embodiments of the present invention, the tumour associated antigen may comprise any of the following fusion proteins:

A fusion protein of Lipoprotein D fragment, MAGE 1 fragment, and histidine tail; fusion protein of NS1-MAGE3, and Histidine tail; fusion protein of CLYTA-MAGE1-Histidine; fusion protein of CLYTA-MAGE3-Histidine.

A further embodiment of the present invention comprises utilising a nucleic acid immunotherapeutic, which comprises a nucleic acid molecule encoding a Mage specific tumour associated antigens as described herein. Such sequences may be inserted into a suitable expression vector and used for DNA/RNA vaccination. Microbial vectors expressing the nucleic acid may also be used as vectored delivered immunotherapeutics. Such vectors include for example, poxvirus, adenovirus, alphavirus and listeria.

Conventional recombinant techniques for obtaining nucleic acid sequences, and production of expression vectors of are described in Maniatis et al., Molecular Cloning—A Laboratory Manual; Cold Spring Harbor, 1982-1989.

For protein based immunotherapeutics the proteins of the present invention are provided either in a liquid form or in a lyophilised form.

It is generally expected that each human dose will comprise 1 to 1000 μg of protein, and preferably 30-300 μg.

The method(s) as described herein may comprise a composition further comprises a vaccine adjuvant, and/or immunostimulatory cytokine or chemokine.

Suitable vaccine adjuvants for use in the present invention are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quit A. Cytokines, such as GM-CSF or interleukin-2, -7, or -12, and chemokines may also be used as adjuvants.

In formulations it may be desirable that the adjuvant composition induces an immune response predominantly of the Th1 type. High levels of Th1-type cytokines (e.g., IFN-γ, TNFα, IL-2 and IL-12) tend to favour the induction of cell mediated immune responses to an administered antigen. According to one embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989. Accordingly, suitable adjuvants that may be used to elicit a predominantly Th1-type response include, for example a combination of monophosphoryl lipid A, such as 3-de-O-acylated monophosphoryl lipid A (3D-MPL) together with an aluminium salt. 3D-MPL or other toll like receptor 4 (TLR4) ligands such as aminoalkyl glucosaminide phosphates as disclosed in WO 98/50399, WO 01/34617 and WO 03/065806 may also be used alone to generate a predominantly Th1-type response.

Other known adjuvants, which may preferentially induce a TH1 type immune response, include TLR9 agonists such as unmethylated CpG containing oligonucleotides. The oligonucleotides are characterised in that the CpG dinucleotide is unmethylated. Such oligonucleotides are well known and are described in, for example WO 96/02555.

Suitable oligionucleotides include:

OLIGO 1:

[Seq ID No 44]

TCC ATG ACG TTC CTG ACG TT (CpG 1826)
OLIGO 2:

[Seq ID No 45]

TCT CCC AGC GTG CGC CAT (CpG 1758)
OLIGO 3:

[Seq ID No 46]

ACC GAT GAC GTC GCC GGT GAC GGC ACC ACG
OLIGO 4

[Seq ID No 47]

TCG TCG TTT TGT CGT TTT GTC GTT (CpG 2006, CpG
7909)
OLIGO 5

[Seq ID No 48]

TCC ATG ACG TTC CTG ATG CT (CpG 1668)

CpG-containing oligonucleotides may also be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800.

The formulation may additionally comprise an oil in water emulsion and/or tocopherol. Another suitable adjuvant is a saponin, for example QS21 (Aquila Biopharmaceuticals Inc., Framingham, Mass.), that may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other suitable formulations comprise an oil-in-water emulsion and tocopherol. A particularly potent adjuvant formulation involving QS21, 3D-MPL and tocopherol in, for example, an oil-in-water emulsion is described in WO 95/17210.

In another embodiment, the adjuvants may be formulated in a liposomal composition.

The amount of 3D-MPL used is generally small, but depending on the immunotherapeutic formulation may be in the region of 1-1000 μg per dose, preferably 1-500 μg per dose, and more preferably between 1 to 100 μg per dose.

In an embodiment, the adjuvant system comprises three immunostimulants: a CpG oligonucleotide, 3D-MPL, & QS21 either presented in a liposomal formulation or an oil in water emulsion such as described in WO 95/17210.

The amount of CpG or immunostimulatory oligonucleotides in the adjuvants or immunotherapeutics of the present invention is generally small, but depending on the immunotherapeutic formulation may be in the region of 1-1000 μg per dose, preferably 1-500 μg per dose, and more preferably between 1 to 100 μg per dose.

The amount of saponin for use in the adjuvants of the present invention may be in the region of 1-1000 μg per dose, preferably 1-500 μg per dose, more preferably 1-250 μg per dose, and most preferably between 1 to 100 μg per dose.

Generally, it is expected that each human dose will comprise 0.1-1000 μg of antigen, preferably 0.1-500 μg, preferably 0.1-100 μg, most preferably 0.1 to 50 μg. An optimal amount for a particular immunotherapeutic can be ascertained by standard studies involving observation of appropriate immune responses in vaccinated subjects. Following an initial vaccination, subjects may receive one or several booster immunisation adequately spaced.

Other suitable adjuvants include Montanide ISA 720 (Seppic, France), SAF (Chiron, Calif., United States), ISCOMS (CSL), MF-59 (Chiron), Ribi Detox, RC-529 (GSK, Hamilton, Mont.) and other aminoalkyl glucosaminide 4-phosphates (AGPs).

Accordingly there is provided an immunogenic composition for use in the method of the present invention comprising an antigen as disclosed herein and an adjuvant, wherein the adjuvant comprises one or more of 3D-MPL, QS21, a CpG oligonucleotide, a polyethylene ether or ester or a combination of two or more of these adjuvants. The antigen within the immunogenic composition may be presented in an oil in water or a water in oil emulsion vehicle or in a liposomal formulation.

In one embodiment, the adjuvant may comprise one or more of 3D-MPL, QS21 and an immunostimulatory CpG oligonucleotide. In an embodiment all three immunostimulants are present. In another embodiment 3D-MPL and QS21 are presented in an oil in water emulsion, and in the absence of a CpG oligonucleotide.

A composition for use in the method of the present invention may comprise a pharmaceutical composition comprising tumour associated antigen as described herein, or a fusion protein, in a pharmaceutically acceptable excipient.

### EXAMPLES

### Example 1

### MAGE008 Mage Melanoma Clinical Trial

In this on-going trial, the recMAGE-A3 protein (recombinant mage fusion protein as shown in Seq Id No 35) is combined with two different immunological adjuvants: either AS02B (QS21, MPL) or AS15 (QS21, MPL and CpG7909). The objectives were to discriminate between the adjuvants in terms of safety profile, clinical response and immunological response.

In this experiment two adjuvant compositions are made up of mixtures of two immunostimulants:
1. QS21 (Purified, naturally occurring saponin molecule from the South-American tree Quillaja Saponaria Molina), and
2. MPL (3 de-O-acetylated monophosphoryl lipid A-detoxified derivative of lipid A, derived from S. minnesota LPS).
   AS02B is an oil-in-water emulsion of QS21 and MPL.

In animal models these adjuvants have been successfully shown to induce both humoral and TH1 types of cellular-mediated immune responses, including CD4 and CD8 T-cells producing IFNα (Moore et al., 1999; Gérard et al., 2001). Moreover, the injection of recombinant protein formulated in this type of adjuvant leads to the induction of a systemic anti-tumor response: indeed, vaccinated animals were shown to be protected against challenges with murine tumor cells genetically engineered to express the tumor antigen, and regressing tumors were shown to be highly infiltrated by CD8, CD4 and NK cells and by macrophages.

The second adjuvant system is AS15: it contains a third immunostimulant, namely CpG7909 (otherwise known as CpG 2006 supra), in addition to MPL and QS21, in a liposome formulation. In animal models (mainly mice), it has been shown that the addition of CpG7909 further improves the induced immune and anti-tumor responses (Krieg and Davis, 2001; Ren et al., 2004). CpG oligodeoxynucleotides (ODNs) directly stimulate dendritic-cell activation through TLR9 triggering. In addition, in mice, the systemic application of CpG7909 greatly increases the infiltration of transferred T-cells into tumors (Meidenbauer et al., 2004).

### Study Overview

1. Design

The MAGE008 trial is:
open
randomized
two-arm (AS02B vs. AS15)
with 68 patients in total.

As described above, the recMAGE-A3 protein is combined with either AS02B or AS15 adjuvant system.

2. Patients Population

The recMAGE-A3 protein is administered to patients with progressive metastatic melanoma with regional or distant skin and/or lymph-node lesions (unresectable stage III and stage IV M1a). The expression of the MAGE-A3 gene by the tumor was assessed by quantitative PCR. The selected patients did not receive previous treatment for melanoma (recMAGE-A3 is given as first-line treatment) and had no visceral disease.

3. Schedule of Immunization

### Method of Treatment Schedules

### Adjuvant Setting

The method of treatment schedule for use in disease in an adjuvant (post-operative) setting may comprise administration of an antigen as described herein according to the following schedules: Administration of antigen at three week intervals for the first 5 to 8 vaccinations, followed at 3 month intervals for the next 8, 9 or more vaccinations.

The antigen may be administered at the exact time frame indicated, or the antigen may be given 1, 2, 3 or 4 days before or after the exact interval, as required or as practical. An example of this schedule is shown in the table below:
Induction: 5 vaccinations at intervals of 3 weeks for example Weeks 0, 3, 6, 9, 12 or Weeks 0, 6, 9, 12
Maintenance: 9 vaccinations at intervals of 3 months
   Alternatively, the vaccinations may be given initially at 2 week intervals, for example 6 injections at two week intervals followed by appropriate maintenance therapy.

### Active Disease

The method of treatment schedule for use in active or unresectable disease, for example in melanoma cancer, comprising: administration of an antigen as described herein at two or three week intervals for the first six months to one year of treatment. A schedule may comprise the following pattern of injections: the antigen may be given at two week intervals for the first 4 to 10 vaccinations, followed by 3 week intervals for the next 4 to 10 vaccinations, then at 6 week intervals for the next 3 to 7 vaccinations. Long term treatment may then continue with vaccinations at 3 month intervals for 3 to 5 vaccinations, followed by 6 month intervals for the next 3 to 5 vaccinations.

The antigen may be administered at the exact time frame indicated, or the antigen may be given 1, 2, 3 or 4 days before or after the exact interval, as required or as practical.

An example of this schedule is shown in the table below:
Cycle 1: 6 vaccinations at intervals of 2 weeks (Weeks 1, 3, 5, 7, 9, 11)
Cycle 2: 6 vaccinations at intervals of 3 weeks (Weeks 15, 18, 21, 24, 27, 30)
Cycle 3: 4 vaccinations at intervals of 6 weeks (Weeks 34, 40, 46, 52)
Long Term Treatment: 4 vaccinations at intervals of 3 months, for example followed by 4 vaccinations at intervals of 6 months
   For both of the above treatment regimes additional vaccinations may be given after treatment, as required.

In order to screen potential participants in the above clinical trial we received biopsies of the tumor, prior to any immunization, as frozen tumor samples. From these samples we extracted RNA for the quantitative PCR. The quality of this purified RNA was extremely high and it was suitable for microarray analysis. We therefore analyzed the tumor samples by microarrays. The goal was to identify a set of genes associated with the clinical response so that patients likely to benefit from this antigen-specific cancer immunotherapeutic are properly identified and selected. Gene profiling has been performed only on biopsies from patients who signed the informed consent for microarray analysis.

### Materials and Methods

### Tumor Specimens

30 tumor specimens (pre-vaccination) were used from the Mage008 Mage-3 melanoma clinical trial. These were fresh frozen preserved in the RNA stabilizing solution RNAlater.

### RNA Purification

Tumoral total RNA was purified using the Tripure method—Tripure extraction (Roche Cat. No. 1 667 165). The protocols provided were followed subsequently by the use of an RNeasy Mini kit—clean-up protocol with DNAse treatment (Qiagen Cat. No. 74106). http://www1.qiagen.com/literature/handbooks/PDF/RNAStabilizationAndPurification/FromAnim alAndPlantTissuesBacteriaYeastAndFungi/RNY_Mini/1035969_HB.pdf. www.roche-applied-science.com/PROD_INF/MANUALS/napi_man/pdf/chapter4/page_{—}152-158.pdf

### RNA Quality Control

Quantification of RNA was initially completed using optical density at 260 nm and Quant-IT RiboGreen RNA assay kit (Invitrogen—Molecular probes R11490). http://probes.invitrogen.com/media/pis/mp11490.pdf.

The Quality of the 28s and 18s ribosomal RNA peaks were assessed by use of the Agilent bioanalyser.

### RNA Labeling and Amplification for Microarray Analysis

Due to the small biopsy size received during the clinical study an amplification method was used in conjunction with the labeling of the RNA for microarray analysis.

The Nugen 3′ ovation biotin kit (Labelling of 50 ng of RNA—Ovation biotin system Cat; 2300-12, 2300-60) http://www.nugeninc.com/html/03_products2.html

A starting input of 50 ng of total RNA was used.

### Microarray Chips

The Affymetrix HU-U133.Plus 2.0 gene chips were utilized as these were the most up to date chips (based on the human genome project) available from the supplier. These chips cover about 47,000 potential gene transcripts.

### Microarray Hybridization and Scanning

The hybridized chips were washed and scanned according to the standard Affymetrix protocols:

Affymetrix gene chip expression analysis protocols can be downloaded from http://www.affvmetrix.com/support/technical/manual/expression_manual.affx.

The scanner that has to be used with the Affymetrix microarray workstation is http://www.affymetrix.com/products/instruments/specific/scanner_{—}3000.affx

### Data Normalization

The fluorescent scanned data was then normalized using RMA to allow comparisons between individual chips. The following reference describes this method.

Irizarry R A, Hobbs B, Collin F, Beazer-Barclay Y D, Antonellis K J, Scherf U, Speed T P. Exploration, normalization, and summaries of high density oligonucleotide array probe level data. Biostatistics. 2003 April; 4(2):249-64.

### Data Analysis

Three independent methods were used to generate gene lists to distinguish responder from non-responding patients. The methods are 1. Spotfire, 2. Baldi B H and 3. Arrayminer. The Baldi analysis method is available from the following website. http://www.igb.uci.edu/˜pfbaldi/software_and_servers.htm.

The spotfire software can be purchased from this site http://www.spotfire.com. The arrayminer software is available for purchase at http://www.optimaldesign.com/ArrayMiner/ArrayMiner.htm.

All data analysis was performed with the use of the R statistics software (available at www.r-project.org) and various Bioconductor packages for R (available at www.bioconductor.org). The raw data were pre-processed for background correction, normalisation and probe summarisation by the RMA (Irizarry R A, Hobbs B, Collin F, Beazer-Barclay Y D, Antonellis K J, Scherf U, Speed T P. Exploration, normalization, and summaries of high density oligonucleotide array probe level data. Biostatistics. 2003 April; 4(2):249-64)

The differential gene expression between any two groups was computed by two different statistical methods, the RankProduct (Breitling, R., Armengaud, P., Amtmann, A., and Herzyk, P. (2004) Rank Products: A simple, yet powerful, new method to detect differentially regulated genes in replicated microarray experiments, FEBS Letter, 57383-92) and the CyberT (P. Baldi and A. D. Long, “A Bayesian Framework for the Analysis of Microarray Expression Data: Regularized t-Test and Statistical Inferences of Gene Changes”, Bioinformatics, 17, 6, 509-519, (2001)) methods.

In each of them, a correction for multiple testing was done with the method of Benjamini-Hochberg (Y. Benjamini and Y. Hochberg, Controlling the false discovery rate: a practical and powerful approach to multiple testing, J. Roy. Stat. Soc. B 57 (1995)), and the threshold of 5% was chosen for the false discovery rate; thus, the genes had to have a corrected p-value less or equal to 0.05 to be considered as differentially expressed. Baldi-BH is equivalent to CyberT followed by Benjamini-Hochberg correction.

### 1. Spotfire Analysis

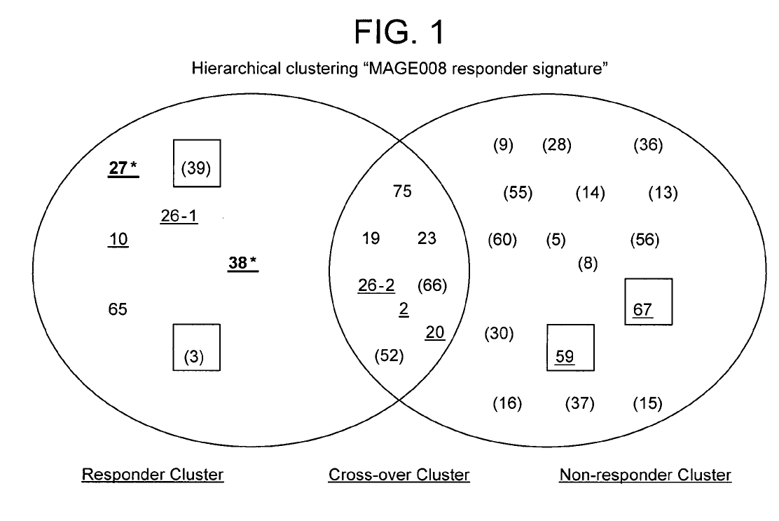

The results of the analysis of patient samples (from the clinical research program named Mage008) are summarized in the FIG. 1.

As a first step, a supervised comparison was performed in order to find a group of genes able to cluster. Two patients that showed clinical response (patient 67 and 59) fell within the cluster of non-repsonders. Two patients who were non-responders (patient 3 and 39) fell within the cluster designated as responders. The responding patients are also found within cross over cluster (ie the areas of overlap in the figure). However, there are two patients who are non-responders that fall within this area of overlap.

In FIG. 1 RESPONDERS were patients 27 and 38 (full/complete clinical responders), patients 2, 10, 20, 26-1, 26-2, 59 & 67 (mixed responders) and patients 19, 23, 65 and 75 (stable disease). NON-RESPONDERS in FIG. 1 were patients 3, 5, 8, 9, 13, 14, 15, 16, 28, 30, 36, 37, 39, 52, 55, 56, 60 and 66.

### 2. Baldi BH Analysis

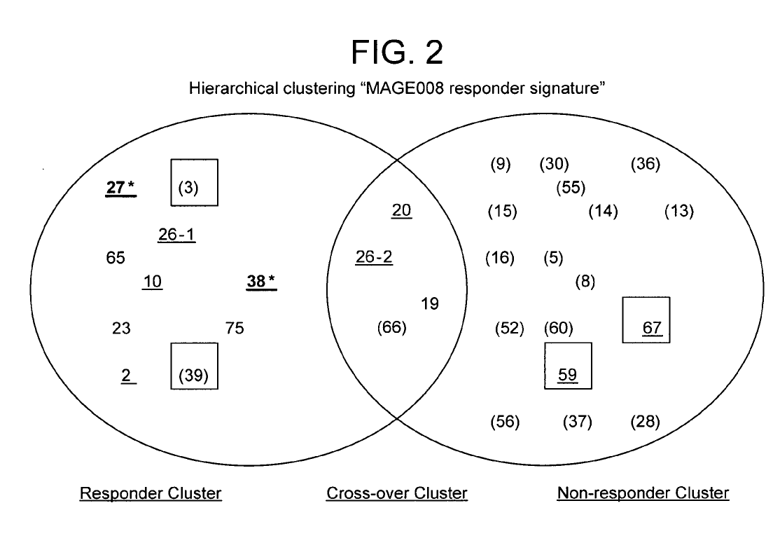

This software is designed for statistical supervised gene classification in microarray experiments. A list of 100 most significant genes were identified that define the two groups. See FIG. 2 below. The clustering is a more effective than with spotfire with the majority of the responders found in the responder cluster. However we still see the same patients non responding (patients 3, 39), and the responding patients (67 and 59) that are located within the wrong clusters.

In FIG. 2 RESPONDERS were patients 27 and 38 (full/complete clinical responders), patients 2, 10, 20, 26-1, 26-2, 59 & 67 (mixed responders) and patients 19, 23, 65 and 75 (stable disease). NON-RESPONDERS in FIG. 2 were patients 3, 5, 8, 9, 13, 14, 15, 16, 28, 30, 36, 37, 39, 52, 55, 56, 60 and 66.

### 3. Arrayminer Analysis

This software is classifies genes by using a “train and test” method. In this method the software first identifies a discriminant set of markers for the training classes, similar to a classical cross-validation study.

Here due to the analysis there is only the responder or non-responder cluster, with no area of overlap. Here again there is mis-classification of samples, in particular patients 59 and 67 who both responded to treatment but in this analysis were in the cluster of non-responders. Patients 3, 14, 39, 52 and 66 were non-responders but were located in the cluster for responders using this analysis.

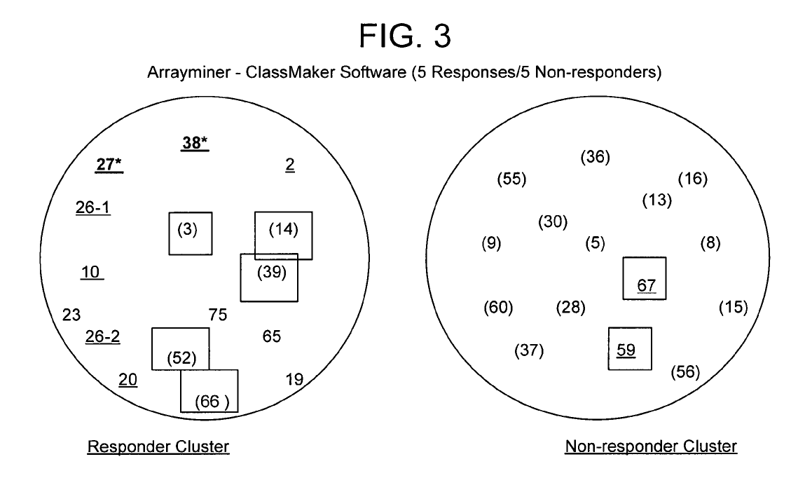

In FIG. 3 RESPONDERS were patients 27 and 38 (full/complete clinical responders), patients 10, 20, 26-1, 26-2, 59 & 67 (mixed responders and patients) 19, 23, 65 and 75 (stable disease). NON-RESPONDERS in FIG. 3 were patients 3, 5, 8, 9, 13, 14, 15, 16, 28, 30, 36, 37, 39, 52, 55, 56, 60 and 66.

### Combination of the Three Methods

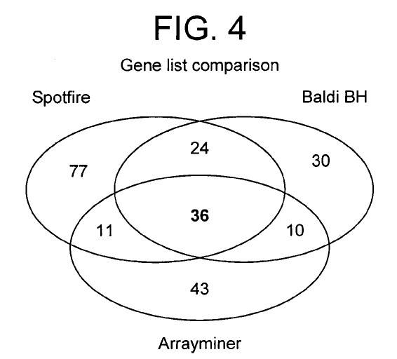

The three methods were used together to find a common set of gene probes that will define the signature with high significance. A venn diagram was created comparing all of the results. See FIG. 4. A list of 36 probesets was found that are common with all three programs. (Table 1B). These 36 probesets were then used for a supervised clustering of the responding and non-responding patients. The genes which are identified by the 36 probes sets form a specific aspect of the invention.

### Fold Change Definition

In programs such as Arrayminer, Fold change of gene expression can be calculated. This is shown in Table 1B and is the change in average gene expression between two groups, for example responder vs non-responder. The statistical value attached to the fold change is calculated and is the more significant in genes where the level of expression is less variable between patients in each group, and difference between groups is larger.

### Gene Expression Shown on a Heat Map

FIG. 5 is a heat map (a diagrammatic respresentation of the expression level of various genes for a given patient), on which can be seen individual expression of genes represented in the Y-Axes by a coloured box and the patients in the X-axes. The expression values of each gene in all samples are normalized to a mean of zero and a standard deviation of 1, and these normalized values are coded into a gradient of colors.

Affymetrix arrays have 11 probe pairs available to interrogate each gene. Ideally, the signal intensity from all of these 11 probes should be equal as they all interrogate the same gene. However, there are enormous differences between individual probes in a probe set. The pattern of the probe signals in a probe set is called a “probe response pattern”. The Robust Multi-array Average” (RMA) software uses, model-based algorithms to incorporate information from multiple microarrays to calculate the expression of a gene. After proper correction for background, and quantile normalisation of the probe intensities, the probe response pattern is fitted over multiple arrays an additive model in RMA software. These algorithms use the fitted models to detect abnormally behaving probes, which are subsequently excluded for calculating gene expression. Therefore, gene expression from these model-based algorithms can be expected to provide more reproducible results. RMA use a stochastic model to estimate gene expression which is potentially a better way of estimating gene expression. Algorithms are implemented in RMA software.

FIG. 4: The predictive 36 probesets correspond mainly to upregulation of genes related to immune infiltration and activation. These genes include HLA class II, Interleukin-2 receptor gamma, T cell receptor genes (TRBV19, TRAT1, TRGC2), granzyme, and CD69.

### Increase in Treatment Efficacy

As described herein, gene profiling has allowed the identification of a subset of patients likely to respond to recMAGE-A3 treatment. This subset corresponds to ˜30% of the patient population. Patients having the gene profile show far higher efficacy levels could be obtained. Indeed, with the early identification of patients susceptible for anti-MAGE-A3 immunization, the efficacy of the treatment will probably increase to levels far higher than other current cancer treatments. This may also lead to a better compliance by the patient. For example, in 100 patients with melanoma, 60 will be found to express MAGE-A3. If these patients all receive recMAGE-A3 injections, 10 out of them will respond; leading to a 15% efficacy rate. However, if gene profile is performed on the 60 MAGE-A3-expressing patients, only the 14 out of them who are predicted to be susceptible to this treatment will receive recMAGE-A3 injections. The other 46 patients will receive another treatment. 10 out of these 14 patients will respond to the treatment, leading to a 71% efficacy rate.

### Gene Profile in Other Tumour Types

This gene profiling has also been performed in a NSCLC study. However, as this study is blinded, only preliminary data are available. It is thought that the signature observed in melanoma is also present in NSCLC, stages IB and II. Preliminary data suggests approximately 30% of the patients present a gene profile signature characteristic of immune response and immune cell infiltration and that suggests they will respond to ASCI treatment.

FIG. 5 shows and expression profile for 30 individual patients for a number of genes.

FIG. 5a shows the expression profiles for 31 patients of two genes.

### Example 2

### Predicting the Clinical Outcome of Patients Using Machine Learning Approaches

### Materials & Methods.

### Tumor Specimens, RNA Purification, Quality Control, Labeling and Amplification for Microarray Analysis

Tumor specimens (pre-vaccination) were used from a Mage-3 melanoma clinical trial (MAGE008). These were preserved, RNA prepared, labeled and amplified as in Example 1 above. The quality of the RNA samples was checked prior to hybridization to the genechip. Only data from samples that were deemed to adequately hybridize to the genechip were used in this Example (61 samples in total).

### Microarray Chips, Hybridizations and Scanning.

As in the material and methods section of the first example above (referred to herein as Example 1), Affymetrix HG-U133.Plus2.0 genechips were employed, and hybridized and scanned as described therein.

### Data Processing & Normalization.

The fluorescent scanned data image was processed and normalized using a R 2.3.1 [1] implementation of GCRMA algorithm [2,3].

### Unspecific Filtering of Gene Expression Matrix

Prior to calculation of differential expression gene expression matrix was filtered in an unspecific (i.e. independently of experimental groups) manner. 4 processes were used:
1. panp filtering: calls indicating whether a gene is expressed in a given sample (present call, P) or not (absent call, A) were derived for all array measures using the panp method implemented in the panp R package [4]. Only probe sets showing at least one P call throughout experimental samples were kept for subsequent calculations.
2. SD filtering: standard deviation (SD) of each probe sets were calculated throughout samples, were only kept probe sets having SDs above the 75th percentile of all SDs.
3. IQR filtering: interquartile range (IQR) of each probe sets were calculated throughout samples, were only kept probe sets having IQRs above the 7th quantile of all IQRs.
4. SH filtering: SD of each probe sets were calculated throughout samples, the midpoint of the SD shorth (the shortest interval containing all of the data) as calculated using the shorth function of genefilter package [5] under R 2.3.1, were only kept probes sets having SD above the shorth for subsequent calculations.

### Calculation of Differential Expression.

The differential expression between the groups of patients showing a clinical benefit after vaccination or not (subsequently called Responder (R) or Non-Responder (NR) groups) was calculated in R 2.3.1 program according to 2 statistical procedures:
1. Regular t-test, as implemented in the genefilter package, and
2. A modified (regularized, moderated) t-test, to account for poor estimates of gene-specific variance under standard t-test, such as the method of Jain et al [6], implemented the LPE R package [7], or the method of Smyth [8], implemented in the limma R package [9], or the method of Baldi and Long [10], also known as Cyber-T [11].

Correction for multiple testing by controlling the false discovery rate (FDR) at 5% was done under R 2.3.1 with Benjamini-Hochberg method [12] implemented in the multitest package [13].

### Gene Profile Normalizations

To make the probe sets having low and high levels of expression comparable and to put an emphasis on differential profiles rather than absolute profiles, data was further normalized at the probe set level. Two gene-level normalizations schemes were used:
1. IQR normalization, where each probe set measure is subtracted by its median over samples and divided by its IQR.
2. Z-score normalization, where each probe set measure is subtracted by its mean over samples and divided by its SD.

### Machine Learning Algorithms

The 14 machine learning algorithms (or predictive rules) interfaced in MLInterfaces package [14] running under R 2.3.1 program were used to train clinical outcome predictive models and to predict the Mage008 patient clinical outcomes, under the reporter lists calculated by the unspecific filtering, differential expression, gene normalization processes. These algorithms were, knn (k-nearest neighbour, kNN, function of class package), nnet (neural network), gbm (generalized boost regression), lvq1 (learning vector quantization 1), naiveBayes (naïve bayes classifier), svm (support vector machine), lda (linear discriminant algorithm), rpart (recursive partitioning), stat.diag.da (diagonal discriminant analysis), randomforest (random forest), bagging, ipredknn (k-nearest neighbour function form ipred package, equivalent to knn form class package), slda (stabilized linear discriminant analysis), pamr (partition around medoids, also known as nearest shrunken centroid classifier). Default parameters were used for all algorithms except for kNN rule where even values of k ranging from 1 to 7 were tested.

Leave-one-out (LOO) scheme of MLinterfaces coded in the xval function was used for cross-validation when appropriate, without re-calculation of reporter list at each cross-validation loop. The misclassification rate was calculated by averaging the number of wrongly predicted samples to the total number of samples of each patient group or class (either R or NR).

Some of the MLIinterfaces interfaced predictive rules (nnet, Ida, naiveBayes) were used outside of the MLInterfaces package as direct calling to make the related classifiers usable on external data sets independently of the data used to train the models.

The MiPP algorithm [15, 16], implemented in the MiPP R library and ran under R 2.3.1, was employed to reduce the number of probe sets involved in the reporter lists. The best reporters out of the submitted list were selected as optimizing the 10-fold cross-validation of linear discriminant analysis predictions of a training set and further reduced to reporters giving an optimized global misclassification error of linear discriminant analysis predictions of a testing set. Global misclassification error or rate was calculated as the ratio of the total number of misclassified patients to the total of set patients.

### Results

### Patient Sample Stratification for Training and Testing Section Definitions

A 31 sample subpart of the full 61 sample data set was used to train and test the classifying model in a first attempt; the remaining 30 samples were kept for subsequent model evaluations. Several training and testing sections were defined from the 31 sample data set subpart. The training sections were used to calculate the reporter list (i.e. the differentially expressed genes to be used in clinical outcome prediction of patient samples) and to set the machine learning algorithm weights for clinical outcome prediction. The testing sections allowed evaluation of the model performance independently of the patients used to construct the predictive model. Model evaluations were also undertaken on the training sections according to a cross-validation procedure.

Training and testing sections from the 31 sample data set subpart were defined as follows:
1. A first training section containing 5 Responders and 5 Non-Responders, leaving 21 patients for the testing section;
2. A second training section of 10 Responders and 11 Non-Responders, leaving 10 patients for the testing section;
3. A last training section containing all 31 patients (13 Responders and 18 Non-Responders). In this last stratification there is no patient left for a testing section, classifying model would only be evaluated by a cross-validation procedure.

### Identification of the Differential Expression Calculation Process Performing Best for Classification.

Gene expression data was pre-processed and normalized according to Material & Methods taking into account the full data set. Differential expressions (DE) between the Responder and Non-Responder groups were calculated for each training/testing section according to various processes.

The DE calculation framework was constructed as follows:
1. Unspecific filtering of gene expression matrix: panp filtering, or not, followed by either SD, IQR or SH filtering, or none.
2. Calculation of DE: either t-test or a modified (regularized, moderated) t-test, such as the method of Jain et al, implemented the LPE R package, or the method of Smyth, implemented in the limma R package, or the method of Baldi and Long (Cyber-T), followed by multiple testing correction, or not.
3. Gene profile normalizations: Either IQR or Z-score normalizations, or none.

The processes minimizing the overall misclassification rates in both training and testing sections in at least two out of the three available training/testing stratification's were selected. The misclassification rate was obtained by comparing the predicted clinical outcome given by a kNN predictive rule to the given clinical outcome.

Seven DE processes were found as minimizing the overall misclassification rate according to the kNN rule:
t-test without correction for multiple testing and irrespective of panp filtering;
SH filter, t-test without correction for multiple testing and irrespective of panp filtering;
SD filter, t-test without correction for multiple testing;
SD filter, t-test without correction for multiple testing, Z-score normalization;
IQR filter, t-test without correction for multiple testing, IQR normalization.

The minimized overall misclassification rates for these processes were as follows:
0% in the 5 Responders to 5 Non-Responders stratification, as evaluated by leave-one-out cross-validation, all patients were therefore correctly classified.
20% in the 21 patients testing section, given by the direct prediction of these patients from the model defined from the 5 Responders to 5 Non-Responders training set. 80% of patients were correctly predicted.
12% for the 13 Responders to 18 Non-Responders segregation, as assessed by cross-validation, being 88% of patient correctly predicted.

The performances for the other training/testing stratification was not found to be informative (i.e. no DE process converging to a minimized misclassification rate), hence not mentioned.

The last process, IQR filter, t-test without correction for multiple testing, IQR normalization, was preferred since the minimized misclassification rates were obtained with a higher value of k (k=5) than other six processes (k=3). It was anticipated that working with higher values of k would facilitate the transposition of the predictive model in further settings, since high k values generate simpler models supposed to be less biased towards a training set.

This process generated a reporter list of 489 Affymetrix probe sets. The reporter list is given in Table 4A, as Affymetrix probe set identifications along with gene names and symbols.

### External Validation: Independent Evaluation of the Best Clinical Outcome Predicting Model.

The best DE calculation and gene expression treatment process (IQR filter, t-test without correction for multiple testing, IQR normalization) was used to build a kNN predictive model (k=5) from 13 Responders/18 Non-responders training section.

The clinical outcome of the independent patients that were previously left away was then directly predicted from this model. Of these 30 independent patients only 15 were informative (i.e. having a characterized clinical outcome). Misclassification rate was calculated on the basis on these 15 informative patients for the model evaluation.

According to this predictive model, misclassification rate was 34%, hence 66% of independent patients had their clinical outcome correctly predicted.

### Improvement of Clinical Outcome Predicting Model Through Other Classification Rules.

On the basis of the best DE (and expression normalization) process as selected with a kNN rule, further classification (predictive) rules were tried to eventually increase the predictive model performance. Beside kNN, 13 other rules were assessed. For each selected DE process that performed for the kNN rule, each further rule was trained on the 13 Responders/18 Non-responders section of the data set, its predictive performance evaluated on the training set by cross-validation and on the independent set of 30 patient samples by direct prediction. Further classification rules did not improve the misclassification rate, as assessed by cross-validation on the training set, the lowest remaining unchanged at 12% of samples miss-predicted (88% of patient assigned to their correct clinical outcome).

However, for the above mentioned preferred DE process, the neural network rule did improve the misclassification rate on the independent set of 15 informative patients out of the 30 available, by reducing it from 34%, as given by KNN rule, to 30% (i.e. one additional patient correctly predicted), while keeping it the lowest on the cross-validation evaluation of the training set. Neural network rule then allows the correct prediction of 70% of patient independently of those used to train the model.

Furthermore, in a further evaluation of the predictive modeling performance, when clinical outcomes were available for all 30 external patient samples, i.e. when all the independent patients were informative for misclassification rate calculation, the lowest misclassification error became 26%, which is 74% of patients were correctly classified. This best performance obtained on a wider data set in terms of informative sample number was given by a Ida rule. A naiveBayes rule was also able to give a 28% misclassification rate, correctly prediction the clinical outcome of 72% of patients. However, the misclassification rate using kNN and nnet rules became 35% and 41%, respectively. This performance is based on the use of the gene list and probes of Table 4A.

### Implementation of the Preferred Classifiers, Predicting the Clinical Outcome of Further Patient Biopsies.

To make use on further metastatic melanoma samples of the exemplified neural network classifier, yielding 30% of misclassification on the 15 informative sample external set, the following R code can be used in a R session (a stastical programme):

library(nnet)
set.seed(1234)
predict(nn, data, type=“class”)
where
data is a data frame containing the 489 reporter (Table 4A) expression data of the samples to classify (GCRMA sample normalized and IQR gene normalized). Samples are organized in columns and reporters in rows;
nn is the R object of class nnet shown in Appendix A.
   the Ida (linear discriminant analysis) classifier would be reproduced by programming the following R code chunk:
   library (MASS)
   predict(lda, data, type=“class”)
   where
data is as previously;
lda is the R object of class Ida shown in Appendix B.

The naïve Bayes classifier works under the following R coding:

library(e1071)
predict(nb, data, type=“class”)
where
data is as previously;
nb is the R object of class naiveBayes shown in Appendix C.

### Down-Sizing the 489 Probe Set Reporter List While Maintaining an Identical Classification Performance.

The MiPP algorithm was used to determine if it was possible to reduce the number of reporters involved in the IQR filter, t-test without correction for multiple testing, IQR normalization, based predictive model while keeping the same misclassification rate. Indeed, working under a shorter reporter list would be more convenient for classifier follow-up purposes for instance such as transposition from a microarray based format to a quantitative RT-PCR format.

The 489 probe set microarray data was submitted to the MiPP process: 11 classifying probe sets were selected on the 13 R/18 NR training section as being sufficient to achieve a lowest global misclassification error, and 4 probe sets out of these were further identified as minimal on the 30 informative external sample data set to obtain the lowest global misclassification rate for the independent patients.

The 4 probe set reporter list was then used in leave-one-out cross-validation predictions of the 13 R/18 NR stratification of data as training set and direct prediction the 30 informative patient section as external testing set under all 14 predictive rules. LOO performance was increased for most of the rules (being as high as 3% of misclassification rate for Ida and naiveBayes among other), while misclassification rate stayed 28% on the external data set under svm and gbm rules.

The following table depicts the identity of the genes involved in the 4 probe set model:

| Probe | Gene | Gene |
|---|---|---|
| Set ID | Symbol | Name |
| 207651_at | GPR171 | G protein-coupled receptor 171 |
| 205392_s_at | CCL14 | chemokine (C-C motif) ligand 14 |
| 212233_at | MAP1B | microtubule-associated protein 1B |
| 206204_at | GRB14 | growth factor receptor-bound protein 14. |

### Example 3

### Predicting the Clinical Outcome of Patients Using Quantitative Polymerase Chain Reaction (Q-PCR)

### Material & Methods

### Tumor Specimens and RNA Purification.

30 tumor specimens (pre-vaccination) were used from MAGE-A3 melanoma clinical trial (MAGE008). These were preserved and RNA extracted as in Example 1 above.

cDNA Synthesis and Quantitative PCR Amplification

2 μg of total RNA were retro-transcripted into cDNA using M-MLV reverse transcriptase (Invitrogen) and oligo(dT) or random primers.

The different interesting genes were amplified by quantitative PCR using TaqMan chemistry and 7900 sequence detection system (Applied Biosystems).

Genes were amplified using standard 96 well plates or the TaqMan® Immune Profiling Array (TaqMan Low density arrays—TLDA—Applied Biosystems). TLDA are ready to use 384 well plates pre-coated with primers and probes.

The 7900 apparatus is a fully integrated system for real-time detection of PCR including a 96-well or a TLDA thermal cycler, a laser to induce fluorescence, a charge-coupled device detector, a computer and a real-time sequence detection software.

In standard 96 well plates, cDNA corresponding to 50 ng of total RNA was amplified by polymerase-chain-reaction using the TF,TAQMAN,PCR REAGENT CORE KIT (Applied Biosystems). Primers and probes are listed in Table 5.

For the TaqMan® Immune Profiling Array, cDNA corresponding to only 1 ng of RNA was amplified by polymerase-chain-reaction using the TF,TAQMAN,PCR CORE REAGENT KIT (Applied Biosystems). The genes included in the array are listed in Table 6.

### Data Processing and Normalization

All PCR data were normalized against H3F3A (standard 96 Well plates) or the geometric mean of GUSB and PGK1 (TLDA) house keeping genes (also referred to as constant genes). Expression values were afterwards log transformed.

### Univariate Statistical Analysis

Analyses of variance (ANOVA1) were performed using the SAS software to significantly select the genes able to differentiate responder from non-responder groups. The first group was composed of 14 responder patients, the second one was composed of 15 non-responder patients. Each gene was analyzed independently from the others.

IL7R, CD3D, CD52, UBD, GPR171, GMZK, PRKCQ, STAT4, TRDV2, TRAT1, TRBV19, CD69, INDO1, CD45R, CD45RO, FoxP3, CD20, CCL5, FASLG, GNLY, GZMB, PRF1, IFNG, ICOS, TBX21, CD8A, CD3E, CXCL10, CXCL11, IRF1, TLR7 and CXCR3 genes were selected for their capacity to differentiate both groups. The p-value was <0.00001 for every gene. For every gene, a significant difference was proven between mean (responder/non-responder). The Geomean ratios between both groups were also calculated for all genes (Table 7A) and range from 3.4 to 21.2.

### Correlation Analysis

A correlation matrix (Table8) was calculated between 30 genes showing that all these genes are pretty well correlated.

### Logistic Regression Model

An analysis by logistic regression was performed using Proc logistic (SAS Sofware). Logistic regression analysis is often used to investigate the relationship between binary responses and a set of explanatory variables.

The number of predictors is too large and the use of all possible regression is not feasible. So, stepwise selection was employed. This procedure gives a model with a good value (high) for a specified criterion: Score Chi-square. This is analogous to the use of SSE or R² in multiple linear regression.

The model information are:
Data: 29 patients and 111 genes.
Response variable: clinical status
Number of response level: 2 (responder-non responder)
Mode: binary logit
Optimization technique: Fisher's scoring

The model giving the best value of score Chi-square (Chi-square=16 and pvalue <0.00001) is:

Logit (p)=7.69+5.07 log(PRF1), with p the probability to be responder
Coefficient B0 (7.69) & B1 (5.07) are significant (p<0.005)
Other criterion can be used to test goodness of fit

| | |
|---|---|
| Likehood Ratio | 23.1429 (chi-square) and <0.001 (p-value) |
| Wald | 6.9250 (chi-sqaure) and <0.001 (p-value) |

Like in linear regression, a R² is computed: 0.55.

From the 111 genes, 18 (with only one predictor) are listed in the Table 9. Higher the R2(%) and chi-square score are the better the model/gene.

Using the PRF1 model, it is possible to classify correctly 86.6% of patients.

Table 10 gives the percentage of correct classification calculated using the logistic regression model for some other genes.

Combining more than 1 gene didn't improve the classification performance.

### Genex Analysis

The 30 patients were also classified using the PCA (Principal Component Analysis) and neural network analysis from Genex software with the genes PRF1, GZMB, GNLY, CD8A, PRKCQ, FOXP3, IFNG, CCL5, GPR171 and TRBV19.

For the neural network analysis, 5 responder patients (Patient ID No.s 27, 53, 65, 119, 127) and 5 non responder patients (Patient ID No.s 8, 60, 66, 81, 160) were used as training set. The remaining 20 patients were used as testing set.

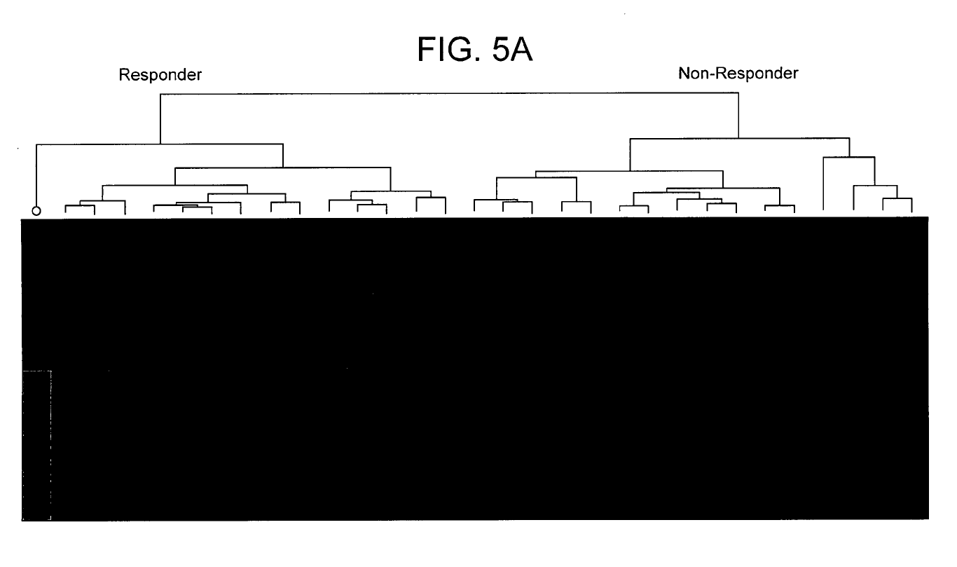

FIG. 6 shows the Principal Component Analysis using PRF1, GZMB, GNLY, CD8A, PRKCQ, FOXP3, IFNG, CCL5, GPR171 and TRBV19 genes.

It can be seen the responders are clustered on the left-hand side of FIG. 6 and the non-responders are clustered on the right-hand side of the figure. Patients labeled 85, 3 and 154 are miss-classified. Therefore, the percentage of “correct” classification is 85% using the PCA.

The neural network approach confirms a correct classification range of 85%.

Comparison: Microarray to Q-PCR data.

17 genes (Table 12) evaluated by the Q-PCR technology were already present in the microarray gene lists. These genes are therefore able to discriminate responder from non responder patients whatever the technology used to detect them. These genes form a specific aspect of the invention.

TABLE 12
Genes present in microarray and Q-PCR list.

| Gene symbol | Gene title |
|---|---|
| CCL5 | chemokine (C-C motif) ligand 5 |
| TRAT1 | T cell receptor associated transmembrane adaptor 1 |
| STAT4 | signal transducer and activator of transcription 4 |
| PRKCQ | protein kinase C, theta |
| GPR171 | G protein-coupled receptor 171 |
| UBD | ubiquitin D |
| CD52 | CD52 molecule |
| CD3D | CD3d molecule, delta (CD3-TCR complex) |
| PRF1 | perforin 1 (pore forming protein) |
| CD8A | CD8a molecule |
| CXCL10 | chemokine (C—X—C motif) ligand 10 |
| CD69 | CD69 molecule |
| TRBV19 | T cell receptor beta variable 19 |
| TRDV2 | T cell receptor delta variable 2 |
| IL7R | interleukin 7 receptor |
| GZMK | granzyme K |
| | PROTEIN-TYROSINE PHOSPHATASE, RECEPTOR- |
| | TYPE, C |
| CD45R | (PTPRC) |

Fourteen genes (Table 13) were able to discriminate responder from non-responder patients using Q-PCR technology were not present in the microarray gene lists. This may be due to the increased sensitivity of the Q-PCR technology compared to the microarray.

TABLE 13
Gene used to classify patients using Q-PCR technology, absent
in Microarray gene lists.

| Gene | |
|---|---|
| symbol | Gene title |
| FASLG | Fas ligand (TNF superfamily, member 6) |
| GNLY | granulysin |
| | granzyme B (granzyme 2, cytotoxic T-lymphocyte-associated |
| | serine |
| GZMB | esterase 1) |
| IFNG | interferon, gamma |
| ICOS | inducible T-cell co-stimulator |
| TBX21 | T-box 21 |
| CD3E | CD3e molecule, epsilon (CD3-TCR complex) |
| CXCL11 | chemokine (C—X—C motif) ligand 11 |
| CXCR3 | chemokine (C—X—C motif) receptor 3 |
| CD20 | CD20 molecule |
| FOXP3 | forkhead box P3 |
| INDO | indoleamine-pyrrole 2,3 dioxygenase |
| IRF1 | interferon regulatory factor 1 |
| TLR7 | toll-like receptor 7 |

Some genes absent in the microarray signature but are known to be expressed by immunological cells and thus are also able to predict the clinical outcome of the patients.

The presence of an immune infiltration into the tumoral tissue seems to be the most important biological event to predict the clinical outcome of the patients rather than only a limited list of genes.

For example, PRF1 and CD8A are present in the microarray gene lists and are expressed by CD8 T lymphocytes. CD8 T lymphocytes express also IFNG that is absent in the microarray gene lists. Nevertheless, a good prediction of the clinical response was achieved using this gene by Q-PCR.

The number of miss-classified patients using the q-PCR data remains in the same range than clustering and KNN approaches using microarray data. Therefore different technologies and softwares can be used to classify the patients.

### Example 4

### Predicting the Clinical Outcome of Patients Using Hierarchical Clustering Approach

### Material & Methods

### Tumor Specimens and RNA Purification.

67 tumor specimens (pre-vaccination) were used from MAGE-A3 melanoma clinical trial (MAGE008). These were preserved, RNA prepared, quality controlled, labeled and amplified as in Example 1 above. The quality of the RNA samples was checked prior to hybridization to the genechip.

### Microarray Chips, Hybridizations and Scanning

As in the material and methods section of the first example above (referred to herein as Example 1), Affymetrix HG-U133.Plus2.0 genechips were employed, and hybridized and scanned as described in Example 1.

### Data Processing & Normalization.

The fluorescent scanned data image was processed and normalized using a R 2.3.1 implementation of GCRMA algorithm as described in Example 2.

### Selection of the Gene List

41 Probe Sets were selected to discriminate responder from non responder patients using Arrayminer software described in Example 1.

Analysis parameters were:
Train and test evaluation
Pre-definition of 2 class
   a. Responder class: PID2, PID65, PID75, PID20, PID10, PID19, PID23, PID26, PID27, PID38, PID67
   b. Non Responder class: PID14, PID52, PID66, PID37, PID16, PID13, PID55, PID56, PID5, PID60, PID8
      wherein PID stands for patient identification No (ie the numerical label given to the patient)
Maximum number of markers per class: 25
Number of markers per couple: 1
Allow multi-class markers: Yes
Use KNN classification: No—Proprietary voting method was used.

A list of 50 probe sets was obtained. 9 Probe sets were removed due the Absence status in all samples. The intensities of the 41 probe sets for the 22 patients listed above are listed in Tables 11A and 11B.

### Prediction of the Clinical Status

The clinical status of the 67 patients was predicted using the hierarchical clustering approach of Spotfire software.

Data were normalized regarding the genes using a Z-score calculation available in Spotfire before performing the Hierarchical clustering.

Calculation options of the hierarchical clustering were:
Clustering method: complete linkage
Similarity measure: Euclidean distance
Ordering function: average value

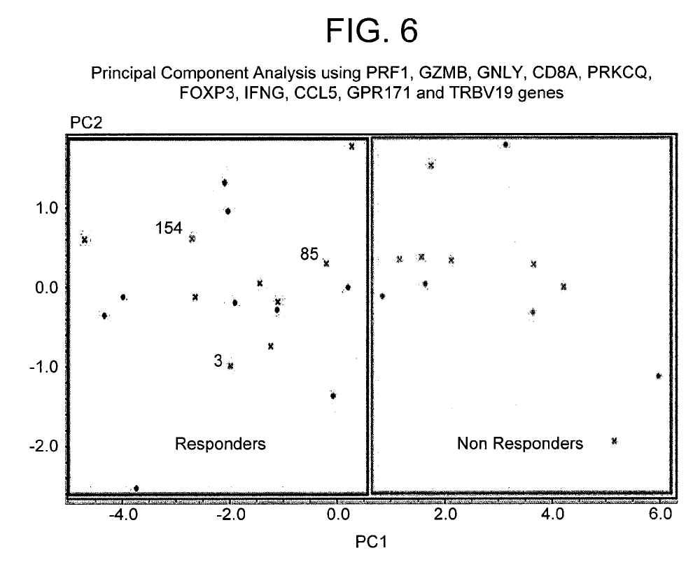

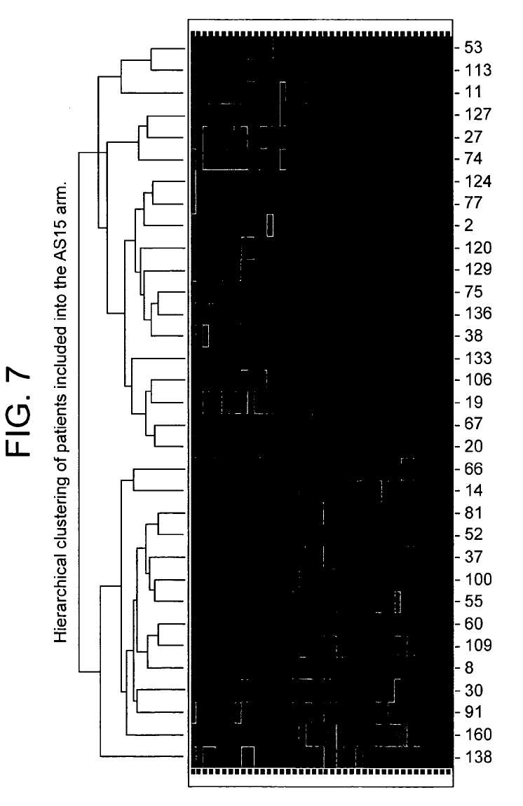

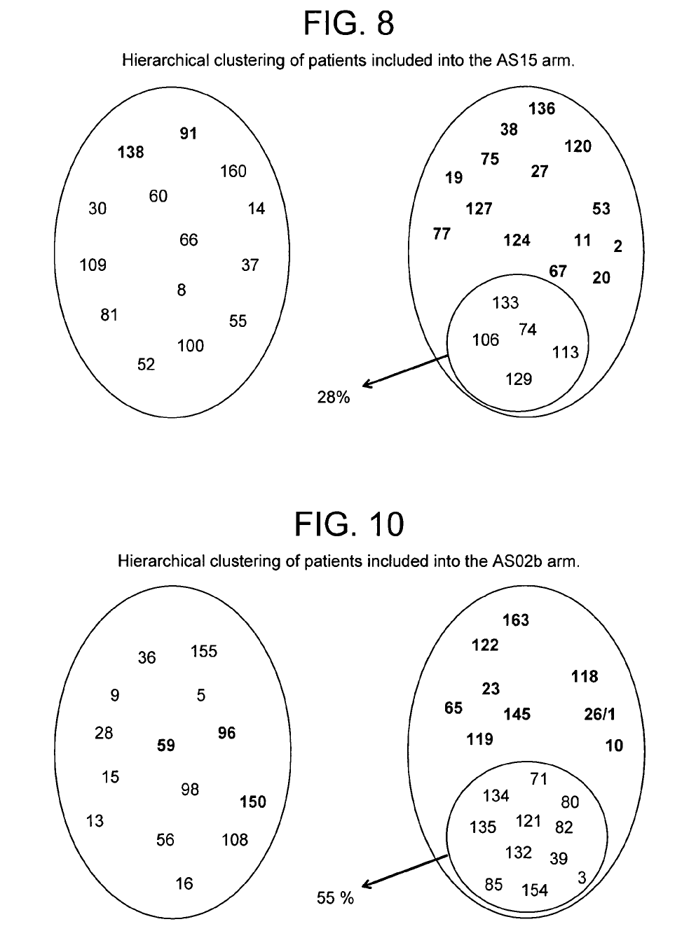

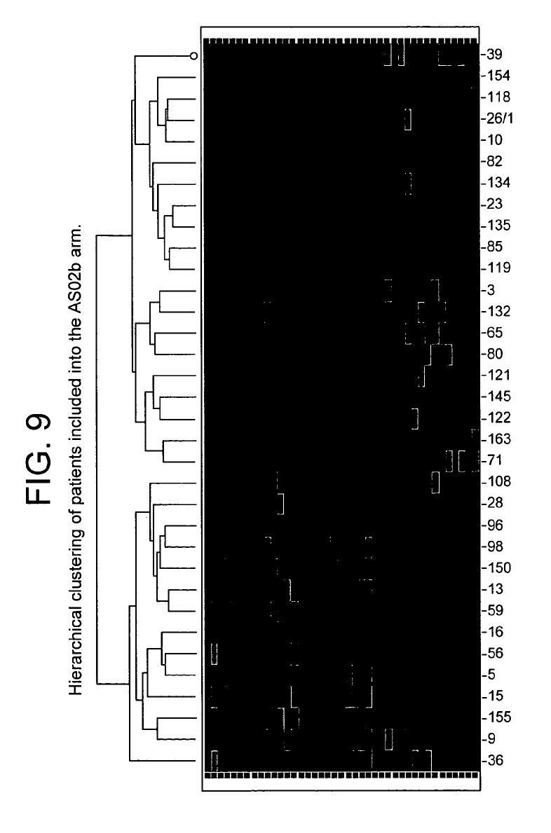

This clustering was performed for patients included into the AS15 (FIGS. 7 and 8) or the AS02B (FIGS. 9 and 10) arms.

Using the hierarchical clustering approach, about 58% of patients have the responder signature whatever the adjuvant group.

100% and 87.5% of patients with a clinical benefit are well clustered respectively in AS15 and in AS02b groups.

71% and 52% of patients with a progressive disease are well clustered respectively in AS15 and in AS02b groups.

In the clinical benefit cluster, 28% and 55% of patients have a progressive disease respectively in AS15 and in AS02b groups. The percentage of patients with a responder gene profile according to the invention who achieved a clinical benefit to the MAGE-A3 vaccination when the AS15 adjuvant was used was larger than the percentage who achieved a clinical benefit to the MAGE-A3 vaccination when AS02b adjuvant was employed in the formulation.

### Example 5

### Inducing a Responder's Profile

Initial biopsy (sampled before irradiation) of patient 59 did not have the responder signature while his second biopsy (sampled after irradiation) had the responder signature (data not shown) suggesting that irradiation of lesions may induce the responder signature.
1. R Development Core Team (2006). R: A language and environment for statistical computing. R Foundation for Statistical Computing, Vienna, Austria. ISBN 3-900051-07-0, URL http://www.R-project.org)
2. Jean (ZHIJIN) Wu and Rafael Irizarry with contributions from James MacDonald Jeff Gentry (2005). germa: Background Adjustment Using Sequence Information. R package version 2.4.1.
3. Wu Z, Irizarry R A, Gentleman R, Martinez-Murillo F, Spencer F: A model-based background adjustment for oligonucleotide expression arrays. Journal of the American Statistical Association 2004, 99:909-917.
4. Peter Warren (2005). panp: Presence-Absence Calls from Negative Strand Matching Probesets. R package version 1.2.0.
5. R. Gentleman, V. Carey and W. Huber (2006). genefilter: genefilter: filter genes. R package version 1.10.1.
6. Jain N, Thatte J, Braciale T, Ley K, O'Connell M, Lee J K. Local-pooled-error test for identifying differentially expressed genes with a small number of replicated microarrays. Bioinformatics. 2003 Oct. 12; 19(15):1945-51.
7. Nitin Jain, Michael O'Connell and Jae K. Lee. Includes R source code contributed by HyungJun Cho <hcho(virginia.edu> (2006). LPE: Methods for analyzing microarray data using Local Pooled Error (LPE) method. R package version 1.6.0. http:/www.r-project.org.
8. Smyth, G. K. Linear models and empirical Bayes methods for assessing differential expression in microarray experiments. Statistical Applications in Genetics and Molecular Biology (2004)₃, No. 1, Article 3.
9. Smyth, G. K. (2005). Limma: linear models for microarray data. In: ‘Bioinformatics and Computational Biology Solutions using R and Bioconductor’. R. Gentleman, V. Carey, S. Dudoit, R. Irizarry, W. Huber (eds), Springer, N.Y., pages 397-420.
10. Baldi P, Long A D. A Bayesian framework for the analysis of microarray expression data: regularized t-test and statistical inferences of gene changes. Bioinformatics. 2001 June; 17(6):509-19.
11. http://visitor.ics.uci.edu/genex/cybert/
12. Benjamini, Y. and Hochberg, Y. (1995) Controlling the false discovery rate: a practical and powerfuil approach to multiple testing. J. Roy. Stat. Soc. B., 57, 289-300.
13. Katherine S. Pollard, Yongchao Ge and Sandrine Dudoit. multtest: Resampling-based multiple hypothesis testing. R package version 1.10.2.
14. Jess Mar, Robert Gentleman and Vince Carey. MLInterfaces: Uniform interfaces to R machine learning procedures for data in Bioconductor containers. R package version 1.4.0.
15. Soukup M, Cho H, and Lee J K (2005). Robust classification modeling on microarray data using misclassification penalized posterior, Bioinformatics, 21 (Suppl): i423-i430.
16. Soukup M and Lee J K (2004). Developing optimal prediction models for cancer classification using gene expression data, Journal of Bioinformatics and Computational Biology, 1(4) 681-694.

TABLE 1A
Gene sequences for and Seq ID No.s for the genes of Table 1

| Seq | | |
|---|---|---|
| ID | | |
| No. | Gene name | Full gene sequence |
| 1 | chromosome 6 open | >gi\|58218985\|ref\|NM_001010923.1\|Homo sapiens chromosome 6 open reading |
| | reading frame 190 | frame 190 (C6orf190), mRNA |
| | | ATACTTACAATTACGAGATTTATATTTGCATTAGTCTCTTTGGCTGGTGGGTAGGGGTGAGAGGCTCTTC |
| | | CTGGATCCCTTATTTTCTACAGGAGAGGAGGAAAACACCTGGGATGCTCCAGTGCTCTTACGCAGATAAT |
| | | GATCATTAACATCAGCCTCTCTGATCAAAGAGCTACTCCACCCCACTCTGGCTGTAGTGTGACATTCCTG |
| | | CCTGCCTGAGGAAGAAATGGTGAGCAGGGGCTGCAATTGCAGACAAGTGTCACCCAGAAGCCACAAGTTT |
| | | CTGTGAGCACCAGGTCTACAAACTACCCAAGGCATAGCAATGGCATTATCACTGGAAGAATTCGTCCACT |
| | | CCCTTGACCTCAGGACCCTACCCAGGGTTCTAGAAATCCAGGCAGGCATCTATCTTGAAGGCTCTATTTA |
| | | TGAAATGTTTGGAAATGAATGCTGTTTTTCAACAGGAGAAGTGATTAAAATTACTGGTCTCAAAGTTAAG |
| | | AAGATCATAGCTGAAATTTGTGAGCAGATTGAAGGTTGTGAGTCTCTACAGCCATTTGAACTGCCTATGA |
| | | ATTTTCCAGGTCTTTTTAAGATTGTGGCTGATAAAACTCCATACCTTACTATGGAAGAAATCACAAGGAC |
| | | CATTCATATTGGACCAAGTAGACTAGGGCATCCTTGCTTCTATCATCAGAAGGATATAAAACTAGAGAAC |
| | | CTCATCATAAAGCAGGGTGAGCAAATCATGCTCAACTCAGTTGAAGAGATTGATGGAGAAATAATGGTGA |
| | | GCTGTGCAGTAGCAAGGAATCATCAAACTCACTCATTTAATTTGCCTTTGTCACAAGAAGGAGAATTCTA |
| | | CGAGTGTGAAGATGAACGTATTTACACTCTAAAGGAGATTGTTGAATGGAAGATTCCTAAGAACAGAACA |
| | | AGAACTGTAAACCTTACAGATTTTTCAAATAAGTGGGACTCAACGAATCCATTTCCTAAAGACTTTTATG |
| | | GTACCCTGATTCTCAAGCCTGTTTATGAAATTCAAGGTGTGATGAAATTTCGAAAAGATATAATCCGCAT |
| | | CCTCCCCAGTCTAGATGTCGAAGTCAAAGACATCACTGATTCTTACGATGCTAACTGGTTTCTTCAGCTG |
| | | TTATCAACAGAAGATCTTTTTGAAATGACTAGTAAAGAGTTCCCCATAGTGACTGAAGTCATAGAAGCAC |
| | | CTGAAGGAAACCACCTGCCCCAAAGCATTTTACAGCCTGGGAAAACCATTGTGATCCACAAAAAGTACCA |
| | | GGCATCAAGAATCTTAGCTTCAGAAATTAGAAGCAATTTTCCTAAAAGACACTTCTTGATCCCCACTAGC |
| | | TATAAAGGCAAGTTCAAGCGGCGACCGAGGGAGTTCCCAACGGCCTATGACCTAGAGATCGCTAAGAGTG |
| | | AAAAGGAGCCTCTTCACGTGGTGGCCACCAAAGCGTTTCATTCCCCTCATGACAAGCTGTCATCCGTATC |
| | | TGTTGGGGACCAGTTTCTGGTGCATCAGTCAGAGACGACTGAAGTCCTCTGTGAGGGAATAAAAAAAGTG |
| | | GTGAATGTTCTGGCCTGTGAAAAAATCCTCAAAAAGTCCTATGAGGCTGCGCTGCTCCCTTTGTACATGG |
| | | AAGGAGGTTTTGTAGAGGTGATTCATGATAAGAAACAGTACCCGATTTCTGAGCTCTGTAAACAGTTCCG |
| | | TTTGCCCTTCAATGTGAAGGTGTCTGTCAGGGATCTTTCCATTGAAGAGGACGTGTTGGCTGCCACACCA |
| | | GGACTGCAGTTGGAGGAGGACATTACAGACTCTTACCTACTCATAAGTGACTTTGCCAACCCCACGGAGT |
| | | GCTGGGAAATTCCTGTGGGCCGCTTGAATATGACTGTTCAGTTAGTTAGTAATTTCTCTAGGGATGCAGA |
| | | ACCATTTCTAGTCAGGACTCTGGTAGAAGAGATCACTGAAGAGCAATATTACATGATGCGGAGATATGAA |
| | | AGCTCAGCCTCACATCCCCCACCTCGCCCTCCGAAACACCCCTCAGTAGAGGAAACAAAGTTAACCCTGC |
| | | TAACCTTAGCAGAAGAAAGGACGGTAGACCTGCCCAAGTCTCCCAAGCGTCATCACGTAGACATAACCAA |
| | | GAAACTTCACCCAAATCAAGCTGGCCTGGATTCAAAAGTACTGATTGGTAGTCAGAATGATTTGGTGGAT |
| | | GAAGAGAAAGAAAGGAGCAACCGTGGGGCCACAGCAATAGCAGAAACATTCAAAAATGAAAAACATCAAA |
| | | AATAACAAGATGTGACAGAAGCCACTTAGGCAGCAAACATAAATGTTGCAGTGAAAAAAGAAGCTAGCCT |
| | | TCTAGCTGAAAAACGAGTATTCCCCAATGGACTCCAGAAGAAACTTGATTCATCGCTGCAAAGGAAAGAA |
| | | CAACCTTAAAACTTTTAACAGATAAAACTTACAGAAACCTATGATATAGAATTCATATAGTCTATTCTGT |
| | | TGTGTCTAAATCTGTAGGCATTGTGTTGTTGTTCTTTAGGACGTATTTATTTAACTTGCACATTTTTTCA |
| | | GATTCTTATTTCTACTACCAACAACTAAGTAATTGGGAAATAATTCTGTATTTCAGTTTCTGAGTAAAAC |
| | | CAGTCTGAAATAGGATAAAAGCCACCAAATATTTTCTTTTTTTTCCAGAATTTGTTTTGCCATTTTTTAG |
| | | TGCTATCATCATTCCTAACAAGACTAACTTACAGAAAAATAATTATATCTGACTGATTTAAAATGTTCAG |
| | | GTTTCTTATCCAAATCCCTTGGAACTATGGAAAGGAGTTTGATTTCACATTCACAGTGTATTTACAAAAT |
| | | ACGCTGTGTCATAAATATGTTTGAATTCCAACAGCCAAAGCCATTGAGAGTCATAGGAGTTTTCCATAAC |
| | | CTTCTCTTCTATGACCCAACAACAAGCTCATGACTGAAATTTCACCAGATTTCTGAGACGATGTCTTAAT |
| | | ATTCTATGTGCTATGTACCAGATAATTCTTTAGATGAATGTTTCTTAGGATTGTAGGAAAATTATCTAGT |
| | | TAATCATAATATTTGATGGAAAGAAAAAGACAATAAAATTGTAATATAATAAATTTGGCTGACAAGAAAC |
| | | CAAAGTGATTCTTAATTAGTATACATCAGAATGATGCTCTTATAGTTGTACCATCTATAAAAATTACTTT |
| | | AAGGGCTCTCACATTTTAATAATTTATCTTATTATGTATTAAGTATACAGGAACAATATTATTTTTCCTT |
| | | TAACAAAATGAAGAGACAGGCTATCTGGTTAATGTTACATAGGAATTTAATAGTAATGCTTGAACTTCAT |
| | | CCATAGATCATACTCTGTACAAAATTTGTTAGCTAACATCCTATCTCATAATTATTTTATGTTTTGTGGA |
| | | GAAATTTGTTGATTTTGTACCAAAGTGTTTCTGAAGACAATAAATTGTGAGTCAACTTTAGAACAAAAAA |
| | | AATTAGAGTTTTTTCAATGTTTATATTCTGATTAAGCTTACTTTACCTTACATTTTTTCTAAGTAACAAT |
| | | GAATCCTGATTTCTAGTGTCCTAAAAATTGCTTAGTGATTTGATTGTGGTAATATCATTTCTTATCTACA |
| | | ATGTCTAAAGTTTTATGGGACAGTTTTTCTTTTATTTATTTTGCCTGTTTGTGCAGATAAGAACAGAAAC |
| | | TTTTCTAAGACCCACATTTGGTTATTGAAGGCCACAGCGAATCTTAACCTAACAGCCTTGACAAACTGCA |
| | | CCATAGGTGTTTTTAGACTCATATAATTTGTTATTTTTCAAACAATAGTGAATAATTAATATTTTTGTTT |
| | | GGAATTTGAGAACAATTAAATTTGTACTTTTAGTAACTACCATTCTTTGATTAGAAAATTAAGAGAATGC |
| | | ATATCTTACTTTGGTTGTAAATTATCAAGGGCTTTCTAATAGAAATCATATATAACATTTCTAAATATAA |
| | | GTCCTTTCACATACTGTGTTTCCAGTTGTCTTGATATTGAAAAGTGTAATAAACTTCATGCTCACCTATT |
| | | GGAGATTTGGGAAGGTTGAAAATAAACTTCCTAATTTTTAAAAAAAAAA |
| 2 | hematopoietic cell- | >gi\|37059786\|ref\|NM_005335.3\|Homo sapiens hematopoietic cell-specific Lyn |
| | specific Lyn | substrate 1 (HCLS1), mRNA |
| | substrate 1 | GTGGACGCGAGGAGCCGGGCGCTTAGAACAGAGGCTTGCACAGGTGGAGATGTGGAAGTCTGTAGTGGGC |
| | | CATGATGTGTCTGTTTCCGTGGAGACCCAGGGTGATGATTGGGACACAGATCCTGACTTTGTGAATGACA |
| | | TCTCTGAAAAGGAGCAACGATGGGGAGCCAAGACCATCGAGGGGTCTGGACGCACAGAACACATCAACAT |
| | | CCACCAGCTGAGGAACAAAGTATCAGAGGAGCATGATGTTCTCAGGAAGAAAGAGATGGAGTCAGGGCCC |
| | | AAAGCATCCCATGGCTATGGAGGTCGGTTTGGAGTAGAAAGAGACCGAATGGACAAGAGTGCAGTGGGCC |
| | | ATGAGTATGTTGCCGAGGTGGAGAAGCACTCTTCTCAGACGGATGCTGCCAAAGGCTTTGGGGGCAAGTA |
| | | CGGAGTTGAGAGGGACAGGGCAGACAAGTCAGCAGTCGGCTTTGATTATAAAGGAGAAGTGGAGAAGCAT |
| | | ACATCTCAGAAAGATTACTCTCGTGGCTTTGGTGGCCGGTACGGGGTGGAGAAGGATAAATGGGACAAAG |
| | | CAGCTCTGGGATATGACTACAAGGGAGAGACGGAGAAACACGAGTCCCAGAGAGATTATGCCAAGGGCTT |
| | | TGGTGGCCAGTATGGAATCCAGAAGGACCGAGTGGATAAGAGCGCTGTCGGCTTCAATGAAATGGAGGCC |
| | | CCGACCACAGCTTATAAGAAGACGACGCCCATAGAAGCCGCTTCTAGTGGTGCCCGTGGGCTGAAGGCGA |
| | | AATTTGAGTCCATGGCTGAGGAGAAGAGGAAGCGAGAGGAAGAGGAGAAGGCACAGCAGGTGGCCAGGAG |
| | | GCAACAGGAGCGAAAGGCTGTGACAAAGAGGAGCCCTGAGGCTCCACAGCCAGTGATAGCTATGGAAGAG |
| | | CCAGCAGTACCGGCCCCACTGCCCAAGAAAATCTCCTCAGAGGCCTGGCCTCCAGTTGGGACTCCTCCAT |
| | | CATCAGAGTCTGAGCCTGTGAGAACCAGCAGGGAACACCCAGTGCCCTTGCTGCCCATTAGGCAGACTCT |
| | | CCCGGAGGACAATGAGGAGCCCCCAGCTCTGCCCCCTAGGACTCTGGAAGGCCTCCAGGTGGAGGAAGAG |
| | | CCAGTGTACGAAGCAGAGCCTGAGCCTGAGCCCGAGCCTGAGCCCGAGCCTGAGAATGACTATGAGGACG |
| | | TTGAGGAGATGGACAGGCATGAGCAGGAGGATGAACCAGAGGGGGACTATGAGGAGGTGCTCGAGCCTGA |
| | | AGATTCTTCTTTTTCTTCTGCTCTGGCTGGATCATCAGGCTGCCCGGCTGGGGCTGGGGCTGGGGCTGTG |
| | | GCTCTGGGGATCTCAGCTGTGGCTCTATATGATTACCAAGGAGAGGGAAGTGATGAGCTTTCCTTTGATC |
| | | CGGACGACGTAATCACTGACATTGAGATGGTGGACGAGGGCTGGTGGCGGGGACGTTGCCATGGCCACTT |
| | | TGGACTCTTCCCTGCAAATTATGTCAAGCTTCTGGAGTGACTAGAGCTCACTGTCTACTGCAACTGTGAT |
| | | TTCCCATGTCCAAAGTGGCTCTGCCTCCACCCCCTCCCTATTCCTGATGCAAATGTCTAACCAGATGAGT |
| | | TTCTGGACAGACTTCCCTCTCCTGCTTCATTAAGGGCTTGCGGCAGAGACAGCATGGGGAAGGAGGTCCC |
| | | CTTCCCCAAGAGTCCTCTCTATCCTGGATGAGCTCATGAACATTTCTCTTGTGTTCCTGACTCCTTCCCA |
| | | ATGAACACCTCTCTGCCACCCCAAGCTCTGCTCTCCTCCTCTGTGAGCTCTGGGCTTCCCAGTTTGTTTA |
| | | CCCGGGAAAGTACGTCTAGATTGTGTGGTTTGCCTCATTGTGCTATTTGCCCACTTTCCTTCCCTGAAGA |
| | | AATATCTGTGAACCTTCTTTCTGTTCAGTCCTAAAATTCGAAATAAAGTGAGACTATGGTTCACCTGTAA |
| | | AAAAAAAAAAAAAAAAAAAAAAAAAAAAAAAAAAAAAAAAAAAAAAAAAAAAAAAAAAAAAAAAAAAAA |
| 3 | interleukin 2 | >gi\|4557881\|ref\|NM_000206.1\|Homo sapiens interleukin 2 receptor, gamma |
| | receptor, gamma | (severe combined immunodeficiency) (IL2RG), mRNA |
| | (severe combined | GAAGAGCAAGCGCCATGTTGAAGCCATCATTACCATTCACATCCCTCTTATTCCTGCAGCTGCCCCTGCT |
| | immunodeficiency) | GGGAGTGGGGCTGAACACGACAATTCTGACGCCCAATGGGAATGAAGACACCACAGCTGATTTCTTCCTG |
| | | ACCACTATGCCCACTGACTCCCTCAGTGTTTCCACTCTGCCCCTCCCAGAGGTTCAGTGTTTTGTGTTCA |
| | | ATGTCGAGTACATGAATTGCACTTGGAACAGCAGCTCTGAGCCCCAGCCTACCAACCTCACTCTGCATTA |
| | | TTGGTACAAGAACTCGGATAATGATAAAGTCCAGAAGTGCAGCCACTATCTATTCTCTGAAGAAATCACT |
| | | TCTGGCTGTCAGTTGCAAAAAAAGGAGATCCACCTCTACCAAACATTTGTTGTTCAGCTCCAGGACCCAC |
| | | GGGAACCCAGGAGACAGGCCACACAGATGCTAAAACTGCAGAATCTGGTGATCCCCTGGGCTCCAGAGAA |
| | | CCTAACACTTCACAAACTGAGTGAATCCCAGCTAGAACTGAACTGGAACAACAGATTCTTGAACCACTGT |
| | | TTGGAGCACTTGGTGCAGTACCGGACTGACTGGGACCACAGCTGGACTGAACAATCAGTGGATTATAGAC |
| | | ATAAGTTCTCCTTGCCTAGTGTGGATGGGCAGAAACGCTACACGTTTCGTGTTCGGAGCCGCTTTAACCC |
| | | ACTCTGTGGAAGTGCTCAGCATTGGAGTGAATGGAGCCACCCAATCCACTGGGGGAGCAATACTTCAAAA |
| | | GAGAATCCTTTCCTGTTTGCATTGGAAGCCGTGGTTATCTCTGTTGGCTCCATGGGATTGATTATCAGCC |
| | | TTCTCTGTGTGTATTTCTGGCTGGAACGGACGATGCCCCGAATTCCCACCCTGAAGAACCTAGAGGATCT |
| | | TGTTACTGAATACCACGGGAACTTTTCGGCCTGGAGTGGTGTGTCTAAGGGACTGGCTGAGAGTCTGCAG |
| | | CCAGACTACAGTGAACGACTCTGCCTCGTCAGTGAGATTCCCCCAAAAGGAGGGGCCCTTGGGGAGGGGC |
| | | CTGGGGCCTCCCCATGCAACCAGCATAGCCCCTACTGGGCCCCCCCATGTTACACCCTAAAGCCTGAAAC |
| | | CTGAACCCCAATCCTCTGACAGAAGAACCCCAGGGTCCTGTAGCCCTAAGTGGTACTAACTTTCCTTCAT |
| | | TCAACCCACCTGCGTCTCATACTCACCTCACCCCACTGTGGCTGATTTGGAATTTTGTGCCCCCATGTAA |
| | | GCACCCCTTCATTTGGCATTCCCCACTTGAGAATTACCCTTTTGCCCCGAACATGTTTTTCTTCTCCCTC |
| | | AGTCTGGCCCTTCCTTTTCGCAGGATTCTTCCTCCCTCCCTCTTTCCCTCCCTTCCTCTTTCCATCTACC |
| | | CTCCGATTGTTCCTGAACCGATGAGAAATAAAGTTTCTGTTGATAATCATC |
| 4 | CD52 antigen | >gi\|68342029\|ref\|NM_001803.2\|Homo sapiens CD52 molecule (CD52), mRNA |
| | (CAMPATH-1 | CTCCTGGTTCAAAAGCAGCTAAACCAAAAGAAGCCTCCAGACAGCCCTGAGATCACCTAAAAAGCTGCTA |
| | antigen)/// | CCAAGACAGCCACGAAGATCCTACCAAAATGAAGCGCTTCCTCTTCCTCCTACTCACCATCAGCCTCCTG |
| | CD52 antigen | GTTATGGTACAGATACAAACTGGACTCTCAGGACAAAACGACACCAGCCAAACCAGCAGCCCCTCAGCAT |
| | (CAMPATH-1 | CCAGCAACATAAGCGGAGGCATTTTCCTTTTCTTCGTGGCCAATGCCATAATCCACCTCTTCTGCTTCAG |
| | antigen) | TTGAGGTGACACGTCTCAGCCTTAGCCCTGTGCCCCCTGAAACAGCTGCCACCATCACTCGCAAGAGAAT |
| | | CCCCTCCATCTTTGGGAGGGGTTGATGCCAGACATCACCAGGTTGTAGAAGTTGACAGGCAGTGCCATGG |
| | | GGGCAACAGCCAAAATAGGGGGGTAATGATGTAGGGGCCAAGCAGTGCCCAGCTGGGGGTCAATAAAGTT |
| | | ACCCTTGTACTTGCAAAAAAAAAAAAAAAAAAA |
| 5 | CD2 antigen (p50), | >gi\|31542293\|ref\|NM_001767.2\|Homo sapiens CD2 molecule (CD2), mRNA |
| | sheep red blood | ACCAACCCCTAAGATGAGCTTTCCATGTAAATTTGTAGCCAGCTTCCTTCTGATTTTCAATGTTTCTTCC |
| | cell receptor/// | AAAGGTGCAGTCTCCAAAGAGATTACGAATGCCTTGGAAACCTGGGGTGCCTTGGGTCAGGACATCAACT |
| | CD2 antigen (p50), | TGGACATTCCTAGTTTTCAAATGAGTGATGATATTGACGATATAAAATGGGAAAAAACTTCAGACAAGAA |
| | sheep red blood | AAAGATTGCACAATTCAGAAAAGAGAAAGAGACTTTCAAGGAAAAAGATACATATAAGCTATTTAAAAAT |
| | cell receptor | GGAACTCTGAAAATTAAGCATCTGAAGACCGATGATCAGGATATCTACAAGGTATCAATATATGATACAA |
| | | AAGGAAAAAATGTGTTGGAAAAAATATTTGATTTGAAGATTCAAGAGAGGGTCTCAAAACCAAAGATCTC |
| | | CTGGACTTGTATCAACACAACCCTGACCTGTGAGGTAATGAATGGAACTGACCCCGAATTAAACCTGTAT |
| | | CAAGATGGGAAACATCTAAAACTTTCTCAGAGGGTCATCACACACAAGTGGACCACCAGCCTGAGTGCAA |
| | | AATTCAAGTGCACAGCAGGGAACAAAGTCAGCAAGGAATCCAGTGTCGAGCCTGTCAGCTGTCCAGAGAA |
| | | AGGTCTGGACATCTATCTCATCATTGGCATATGTGGAGGAGGCAGCCTCTTGATGGTCTTTGTGGCACTG |
| | | CTCGTTTTCTATATCACCAAAAGGAAAAAACAGAGGAGTCGGAGAAATGATGAGGAGCTGGAGACAAGAG |
| | | CCCACAGAGTAGCTACTGAAGAAAGGGGCCGGAAGCCCCAACAAATTCCAGCTTCAACCCCTCAGAATCC |
| | | AGCAACTTCCCAACATCCTCCTCCACCACCTGGTCATCGTTCCCAGGCACCTAGTCATCGTCCCCCGCCT |
| | | CCTGGACACCGTGTTCAGCACCAGCCTCAGAAGAGGCCTCCTGCTCCGTCGGGCACACAAGTTCACCAGC |
| | | AGAAAGGCCCGCCCCTCCCCAGACCTCGAGTTCAGCCAAAACCTCCCCATGGGGCAGCAGAAAACTCATT |
| | | GTCCCCTTCCTCTAATTAAAAAAGATAGAAACTGTCTTTTTCAATAAAAAGCACTGTGGATTTCTGCCCT |
| | | CCTGATGTGCATATCCGTACTTCCATGAGGTGTTTTCTGTGTGCAGAACATTGTCACCTCCTGAGGCTGT |
| | | GGGCCACAGCCACCTCTGCATCTTCGAACTCAGCCATGTGGTCAACATCTGGAGTTTTTGGTCTCCTCAG |
| | | AGAGCTCCATCACACCAGTAAGGAGAAGCAATATAAGTGTGATTGCAAGAATGGTAGAGGACCGAGCACA |
| | | GAAATCTTAGAGATTTCTTGTCCCCTCTCAGGTCATGTGTAGATGCGATAAATCAAGTGATTGGTGTGCC |
| | | TGGGTCTCACTACAAGCAGCCTATCTGCTTAAGAGACTCTGGAGTTTCTTATGTGCCCTGGTGGACACTT |
| | | GCCCACCATCCTGTGAGTAAAAGTGAAATAAAAGCTTTGACTAGAAAAAAAAAAAAAAAAAAAAAAAAAA |
| | | AAAAAAAAAAAAAAAAAAAAAAAAAAAAAAAAAAAAAAA |
| 6 | ubiquitin D | >gi\|50355987\|ref\|NN_006398.2\|Homo sapiens ubiquitin D (UBD), mRNA |
| | | GATTGCTTGAGGAGAGAAGTATGTGATCAGAAAGCATTCTTTGTCTATTAACTCCTGCCCAGCAAAAGTG |
| | | AAAGAAAATTCATGGGAGCATGCAAGAACAAAGAGCACAGCAAAGCTGGACAAACACAGCAATCCAGGCA |
| | | GGGGATTTCCAACTCAACTCTGGTATGTAAGCTGCATGCAAAGTCCTTTTTCTGTCTCTGGTTTCTGGCC |
| | | CCTTGTCTGCAGAGATGGCTCCCAATGCTTCCTGCCTCTGTGTGCATGTCCGTTCCGAGGAATGGGATTT |
| | | AATGACCTTTGATGCCAACCCATATGACAGCGTGAAAAAAATCAAAGAACATGTCCGGTCTAAGACCAAG |
| | | GTTCCTGTGCAGGACCAGGTTCTTTTGCTGGGCTCCAAGATCTTAAAGCCACGGAGAAGCCTCTCATCTT |
| | | ACGGCATTGACAAAGAGAAGACCATCCACCTTACCCTGAAAGTGGTGAAGCCCAGTGATGAGGAGCTGCC |
| | | CTTGTTTCTTGTGGAGTCAGGTGATGAGGCAAAGAGGCACCTCCTCCAGGTGCGAAGGTCCAGCTCAGTG |
| | | GCACAAGTGAAAGCAATGATCGAGACTAAGACGGGTATAATCCCTGAGACCCAGATTGTGACTTGCAATG |
| | | GAAAGAGACTGGAAGATGGGAAGATGATGGCAGATTACGGCATCAGAAAGGGCAACTTACTCTTCCTGGC |
| | | ATCTTATTGTATTGGAGGGTGACCACCCTGGGCATGGGGTGTTGGCAGGGGTCAAAAAGCTTATTTCTTT |
| | | TAATCTCTTACTCAACGAACACATCTTCTGATGATTTCCCAAAATTAATGAGAATGAGATGAGTAGAGTA |
| | | AGATTTGGGTGGGATGGGTAGGATGAAGTATATTGCCCAACTCTATGTTTCTTTGATTCTAACACAATTA |
| | | ATTAAGTGACATGATTTTTACTAATGTATTACTGAGACTAGTAAATAAATTTTTAAGCCAA |
| 7 | signal transducer | >gi\|21618332\|ref\|NM_003151.2\|Homo sapiens signal transducer and activator |
| | and activator of | of transcription 4 (STAT4), mRNA |
| | transcription 4 | AGAGGACGCCCGGTGAAGGGGCTCCAGCCTGGCAGTTTCTGCGTGTTAGCATTTCTAGAATAGAGTGGGT |
| | | GGGAACTGACCCAAGTAAAGTCCCAGAGACTCGAACACTGACGCACAGGAAAGCCTCAAGTGGGAGGAGA |
| | | AATGCAAATCCCCTACTGATGATGGCGTCAGCGGCTTTCTCCTAGGGACTGTGAGGGGCGCTTCTGACTT |
| | | TGGACTTGAGCACTGCCTGGGACCTGTGCTGAGAGAGCGCTAGCATGTCTCAGTGGAATCAAGTCCAACA |
| | | GTTAGAAATCAAGTTTTTGGAGCAGGTGGATCAATTCTATGATGACAACTTTCCCATGGAAATTCGGCAT |
| | | CTGTTGGCCCAATGGATTGAAAATCAAGACTGGGAGGCAGCTTCTAACAATGAAACCATGGCAACGATTC |
| | | TTCTTCAAAACTTGTTAATACAACTGGATGAACAGTTAGGTCGTGTTTCCAAAGAGAAAAACCTACTCTT |
| | | GATACACAATCTAAAAAGAATTAGGAAGGTCCTTCAGGGAAAATTTCATGGAAATCCAATGCATGTAGCT |
| | | GTGGTTATTTCAAACTGTTTAAGGGAAGAGAGGAGAATATTGGCTGCAGCCAACATGCCTGTCCAGGGGC |
| | | CTCTAGAGAAATCCTTACAAAGTTCTTCAGTTTCAGAAAGACAGAGGAATGTGGAGCACAAAGTGGCTGC |
| | | CATTAAAAACAGTGTGCAGATGACAGAACAAGATACCAAATACTTAGAAGATCTGCAAGACGAATTTGAC |
| | | TACAGGTATAAAACAATTCAGACAATGGATCAGAGTGACAAGAATAGTGCCATGGTGAATCAGGAAGTTT |
| | | TGACACTGCAGGAAATGCTTAACAGCCTCGATTTCAAGAGAAAGGAGGCTCTCAGTAAAATGACCCAAAT |
| | | CATCCATGAGACAGACCTGTTAATGAACACCATGCTCATAGAAGAGCTGCAAGACTGGAAGCGGCGGCAG |
| | | CAAATCGCCTGCATCGGGGGTCCACTCCACAATGGGCTCGACCAGCTTCAGAACTGCTTTACACTATTGG |
| | | CAGAAAGTCTTTTCCAACTGAGAAGGCAATTGGAGAAACTAGAGGAGCAATCTACCAAAATGACATATGA |
| | | AGGTGATCCCATTCCAATGCAAAGAACTCACATGCTAGAAAGAGTCACCTTCTTGATCTACAACCTTTTC |
| | | AAGAACTCATTTGTGGTTGAGCGACAGCCATGTATGCCAACCCACCCTCAGAGGCCGTTGGTACTTAAAA |
| | | CCCTAATTCAGTTCACTGTAAAACTAAGGCTACTAATAAAATTGCCAGAACTAAACTATCAGGTAAAGGT |
| | | TAAGGCATCAATTGACAAGAATGTTTCAACTCTAAGCAACCGAAGATTTGTACTTTGTGGAACTAATGTC |
| | | AAAGCCATGTCTATTGAAGAATCTTCCAATGGGAGTCTCTCAGTAGAATTTCGACATTTGCAACCAAAGG |
| | | AAATGAAGTCCAGTGCTGGAGGTAAAGGAAATGAGGGCTGTCACATGGTGACTGAAGAACTTCATTCCAT |
| | | AACGTTTGAAACACAGATCTGCCTCTATGGCCTGACCATAGATTTGGAGACCAGCTCATTGCCTGTGGTG |
| | | ATGATTTCCAATGTCAGTCAGTTACCTAATGCTTGGGCATCCATCATTTGGTACAACGTGTCAACCAACG |
| | | ATTCCCAGAACTTGGTTTTCTTTAATAATCCTCCACCTGCCACATTGAGTCAACTACTGGAGGTGATGAG |
| | | CTGGCAGTTTTCATCGTACGTTGGTCGTGGTCTTAACTCAGATCAACTCCATATGCTGGCAGAGAAGCTT |
| | | ACAGTCCAATCTAGCTACAGTGATGGTCACCTCACCTGGGCCAAGTTCTGCAAGGAACATTTACCTGGTA |
| | | AATCATTTACCTTTTGGACATGGCTTGAAGCAATATTGGATCTAATTAAGAAACACATTCTTCCCCTTTG |
| | | GATTGATGGGTATGTCATGGGCTTTGTTAGCAAAGAGAAGGAACGGCTGTTGCTAAAGGATAAAATGCCT |
| | | GGCACCTTTTTATTAAGATTCAGTGAAAGCCATCTCGGAGGAATAACTTTCACCTGGGTGGACCATTCTG |
| | | AAAGTGGGGAAGTGAGATTCCACTCTGTAGAACCCTACAATAAAGGCCGGTTGTCTGCTCTGCCATTCGC |
| | | TGACATCCTGCGAGACTACAAAGTTATTATGGCTGAAAACATTCCTGAAAACCCTCTGAAGTACCTATAT |
| | | CCTGACATTCCCAAAGACAAAGCCTTCGGTAAACACTACAGCTCTCAGCCTTGCGAAGTTTCAAGACCAA |
| | | CAGAAAGGGGTGACAAAGGTTATGTTCCTTCTGTTTTTATCCCCATCTCAACAATCCGAAGTGATTCAAC |
| | | AGAGCCACATTCTCCATCAGACCTTCTTCCCATGTCTCCAAGTGTGTATGCGGTGTTGAGAGAAAACCTG |
| | | AGTCCCACAACAATTGAAACTGCAATGAAGTCTCCTTATTCTGCTGAATGACAGGATAAACTCTGACGCA |
| | | CCAAGAAAGGAAGCAAATGAAAAAGTTTAAAGACTGTTCTTTGCCCAATAACCACATTTTATTTCTTCAG |
| | | CTTTGTAAATACCAGGTTCTAGGAAATGTTTGACATCTGAAGCTCTCTTCACACTCCCGTGGCACTCCTC |
| | | AATTGGGAGTGTTGTGACTGAAATGCTTGAAACCAAAGCTTCAGATAAACTTGCAAGATAAGACAACTTT |
| | | AAGAAACCAGTGTTAATAACAATATTAACAG |
| 8 | granzyme K | >gi\|73747815\|ref\|NM_002104.2\|Homo sapiens granzyme K (granzyme 3; |
| | (granzyme 3; | tryptase II) (GZMK), mRNA |
| | tryptase II)/// | GATCAACACATTTCATCTGGGCTTCTTAAATCTAAATCTTTAAAATGACTAAGTTTTCTTCCTTTTCTCT |
| | granzyme K | GTTTTTCCTAATAGTTGGGGCTTATATGACTCATGTGTGTTTCAATATGGAAATTATTGGAGGGAAAGAA |
| | (granzyme 3; | GTGTCACCTCATTCCAGGCCATTTATGGCCTCCATCCAGTATGGCGGACATCACGTTTGTGGAGGTGTTC |
| | tryptase II) | TGATTGATCCACAGTGGGTGCTGACAGCAGCCCACTGCCAATATCGGTTTACCAAAGGCCAGTCTCCCAC |
| | | TGTGGTTTTAGGCGCACACTCTCTCTCAAAGAATGAGGCCTCCAAACAAACACTGGAGATCAAAAAATTT |
| | | ATACCATTCTCAAGAGTTACATCAGATCCTCAATCAAATGATATCATGCTGGTTAAGCTTCAAACAGCCG |
| | | CAAAACTCAATAAACATGTCAAGATGCTCCACATAAGATCCAAAACCTCTCTTAGATCTGGAACCAAATG |
| | | CAAGGTTACTGGCTGGGGAGCCACCGATCCAGATTCATTAAGACCTTCTGACACCCTGCGAGAAGTCACT |
| | | GTTACTGTCCTAAGTCGAAAACTTTGCAACAGCCAAAGTTACTACAACGGCGACCCTTTTATCACCAAAG |
| | | ACATGGTCTGTGCAGGAGATGCCAAAGGCCAGAAGGATTCCTGTAAGGGTGACTCAGGGGGCCCCTTGAT |
| | | CTGTAAAGGTGTCTTCCACGCTATAGTCTCTGGAGGTCATGAATGTGGTGTTGCCACAAAGCCTGGAATC |
| | | TACACCCTGTTAACCAAGAAATACCAGACTTGGATCAAAAGCAACCTTGTCCCGCCTCATACAAATTAAG |
| | | TTACAAATAATTTTATTGGATGCACTTGCTTCTTTTTTCCTAATATGCTCGCAGGTTAGAGTTGGGTGTA |
| | | AGTAAAGCAGAGCACATATGGGGTCCATTTTTGCACTTGTAAGTCATTTTATTAAGGAATCAAGTTCTTT |
| | | TTCACTTGTATCACTGATGTATTTCTACCATGCTGGTTTTATTCTAAATAAAATTTAGAAGACTCAAAAA |
| | | AAAAAAAAAAAAAAAAAAAAAAAA |
| 9 | CD3G antigen, gamma | >gi\|4557428\|ref\|NM_000073.1\|Homo sapiens CD3g molecule, gamma (CD3-TCR |
| | polypeptide (TiT3 | complex) (CD3G), mRNA |
| | complex) | GGGCTGCTCCACGCTTTTGCCGGAGACAGAGACTGACATGGAACAGGGGAAGGGCCTGGCTGTCCTCATC |
| | | CTGGCTATCATTCTTCTTCAAGGTACTTTGGCCCAGTCAATCAAAGGAAACCACTTGGTTAAGGTGTATG |
| | | ACTATCAAGAAGATGGTTCGGTACTTCTGACTTGTGATGCAGAAGCCAAAAATATCACATGGTTTAAAGA |
| | | TGGGAAGATGATCGGCTTCCTAACTGAAGATAAAAAAAAATGGAATCTGGGAAGTAATGCCAAGGACCCT |
| | | CGAGGGATGTATCAGTGTAAAGGATCACAGAACAAGTCAAAACCACTCCAAGTGTATTACAGAATGTGTC |
| | | AGAACTGCATTGAACTAAATGCAGCCACCATATCTGGCTTTCTCTTTGCTGAAATCGTCAGCATTTTCGT |
| | | CCTTGCTGTTGGGGTCTACTTCATTGCTGGACAGGATGGAGTTCGCCAGTCGAGAGCTTCAGACAAGCAG |
| | | ACTCTGTTGCCCAATGACCAGCTCTACCAGCCCCTCAAGGATCGAGAAGATGACCAGTACAGCCACCTTC |
| | | AAGGAAACCAGTTGAGGAGGAATTGAACTCAGGACTCAGAGTAGTCCAGGTGTTCTCCTCCTATTCAGTT |
| | | CCCAGAATCAAAGCAATGCATTTTGGAAAGCTCCTAGCAGAGAGACTTTCAGCCCTAAATCTAGACTCAA |
| | | GGTTCCCAGAGATGACAAATGGAGAAGAAAGGCCATCAGAGCAAATTTGGGGGTTTCTCAAATAAAATAA |
| | | AAATAAAAACAAATACTGTGTTTCAGAAGCGCCACCTATTGGGGAAAATTGT |
| 10 | G protein-coupled | >gi\|31377771\|ref\|NM_013308.2\|Homo sapiens G protein-coupled receptor 171 |
| | receptor 171 | (GPR171), mRNA |
| | | GGTTGCTACTGCTGCGGCTAACCAAACAGCTCATGCTTCTCTGAAGACTTGCAGCAAGGTTTGCTGAGGC |
| | | TCACAGAAGATAGCCCCAGTGTTTTGGAGTGGTTTTGAATGTGATTCTGAGATCAGACTGACTGAGCTGG |
| | | AATCCTGGCTTTATATCTTACCAGCTACACAACCTTGGAGTCTTAGAAATTTTTTCTTTTCAATAAGCAG |
| | | TCATCCTTACTTTCCCTCAAGATGACAAACAGTTCGTTCTTCTGCCCAGTTTATAAAGATCTGGAGCCAT |
| | | TCACGTATTTTTTTTATTTAGTTTTCCTTGTTGGAATTATTGGAAGTTGTTTTGCAACCTGGGCTTTTAT |
| | | ACAGAAGAATACGAATCACAGGTGTGTGAGCATCTACTTAATTAATTTGCTTACAGCCGATTTCCTGCTT |
| | | ACTCTGGCATTACCAGTGAAAATTGTTGTTGACTTGGGTGTGGCACCTTGGAAGCTGAAGATATTCCACT |
| | | GCCAAGTAACAGCCTGCCTCATCTATATCAATATGTATTTATCAATTATCTTCTTAGCATTTGTCAGCAT |
| | | TGACCGCTGTCTTCAGCTGACACACAGCTGCAAGATCTACCGAATACAAGAACCCGGATTTGCCAAAATG |
| | | ATATCAACCGTTGTGTGGCTAATGGTCCTTCTTATAATGGTGCCAAATATGATGATTCCCATCAAAGACA |
| | | TCAAGGAAAAGTCAAATGTGGGTTGTATGGAGTTTAAAAAGGAATTTGGAAGAAATTGGCATTTGCTGAC |
| | | AAATTTCATATGTGTAGCAATATTTTTAAATTTCTCAGCCATCATTTTAATATCCAATTGCCTTGTAATT |
| | | CGACAGCTCTACAGAAACAAAGATAATGAAAATTACCCAAATGTGAAAAAGGCTCTCATCAACATACTTT |
| | | TAGTGACCACGGGCTACATCATATGCTTTGTTCCTTACCACATTGTCCGAATCCCGTATACCCTCAGCCA |
| | | GACAGAAGTCATAACTGATTGCTCAACCAGGATTTCACTCTTCAAAGCCAAAGAGGCTACACTGCTCCTG |
| | | GCTGTGTCGAACCTGTGCTTTGATCCTGTCCTGTACTATCACCTCTCAAAAGCATTCCGCTCAAAGGTCA |
| | | CTGAGACTTTTGCCTCACCTAAAGAGACCAAGGCTCAGAAAGAAAAATTAAGATGTGAAAATAATGCATA |
| | | AAAGACAGGATTTTTGTGCTACCAATTCTGGCCTTACTGGACCATAAAGTTAATTATAGCTTTGAAAGAT |
| | | AAAAAAAAAAAAAAAAACAAAAAAAAACTCAGTATGAAAAAATACAGTTAGCTAGCAAATATGGACAGGT |
| | | TTACTTAGAAATCCTGTTTCTAAATGCAAGTCAAGCTTTATTGTTAGGCTTGCTGCTACTCATTAACCCA |
| | | AATATTTGTACAAAAAACTAAAGAGTCTCATTGAACGAATGTAAAATCCTGCAATATCCTTGAAATCCAA |
| | | AAGAGGTCCATGACATAGACCCAAAGGTATTCATGAGTTATTCATTTAAATGCCTGGAACTGACTTCTTG |
| | | ATAAAAATATAAAAAATAATTTCCATGTAAGTTACCAGAAAGCCCACCAGCAACATAATTTTAAAGCCTT |
| | | TCGGATTACTTTTAAAAAATGCAGCTTACATATAACAACTTGTGCCTATTTTATTTCTAATCTATCACTT |
| | | CAAAAGATGGTAATCTTTCAACTCATTATTCCTCCAATTTTTAATGTCGAATTTTTTTCTAACACAATAA |
| | | CCAAAAGCTTTTATTTATAAAAAGGCTTGAAAAATATAAAAAAAAAAAAAAAAAAAAAAAAAAAAAAAAA |
| | | AAAAAAAAAAAAAAAAAAAAAAAAAAAAAAAAAAA |
| 11 | Protein kinase C, | >gi\|89353296\|ref\|NM_001183.4\|Homo sapiens ATPase, H+ transporting, |
| | beta 1 | lysosomal accessory protein 1 (ATP6AP1), mRNA |
| | | GGGGGCAACGGTCACCTGATCTGCGGCTGTCGAGGCCGCTGAGGCAGTGGAGGCTGAGGCTATGATGGCG |
| | | GCCATGGCGACGGCTCGAGTGCGGATGGGGCCGCGATGCGCCCAGGCGCTCTGGCGCATGCCGTGGCTGC |
| | | CGGTGTTTTTGTCGTTGGCGGCGGCGGCGGCGGCGGCAGCGGCGGAGCAGCAGGTCCCGCTGGTGCTGTG |
| | | GTCGAGTGACCGGGACTTGTGGGCTCCTGCGGCCGACACTCATGAAGGCCACATCACCAGCGACTTGCAG |
| | | CTCTCTACCTACTTAGATCCCGCCCTGGAGCTGGGTCCCAGGAATGTGCTGCTGTTCCTGCAGGACAAGC |
| | | TGAGCATTGAGGATTTCACAGCATATGGCGGTGTGTTTGGAAACAAGCAGGACAGCGCCTTTTCTAACCT |
| | | AGAGAATGCCCTGGACCTGGCCCCCTCCTCACTGGTGCTTCCTGCCGTCGACTGGTATGCAGTCAGCACT |
| | | CTGACCACTTACCTGCAGGAGAAGCTCGGGGCCAGCCCCTTGCATGTGGACCTGGCCACCCTGCGGGAGC |
| | | TGAAGCTCAATGCCAGCCTCCCTGCTCTGCTGCTCATTCGCCTGCCCTACACAGCCAGCTCTGGTCTGAT |
| | | GGCACCCAGGGAAGTCCTCACAGGCAACGATGAGGTCATCGGGCAGGTCCTGAGCACACTCAAGTCCGAA |
| | | GATGTCCCATACACAGCGGCCCTCACAGCGGTCCGCCCTTCCAGGGTGGCCCGTGATGTAGCCGTGGTGG |
| | | CCGGAGGGCTAGGTCGCCAGCTGCTACAAAAACAGCCAGTATCACCTGTGATCCATCCTCCTGTGAGTTA |
| | | CAATGACACCGCTCCCCGGATCCTGTTCTGGGCCCAAAACTTCTCTGTGGCGTACAAGGACCAGTGGGAG |
| | | GACCTGACTCCCCTCACCTTTGGGGTGCAGGAACTCAACCTGACTGGCTCCTTCTGGAATGACTCCTTTG |
| | | CCAGGCTCTCACTGACCTATGAACGACTCTTTGGTACCACAGTGACATTCAAGTTCATTCTCGCCAACCG |
| | | CCTCTACCCAGTGTCTGCCCGGCACTGGTTTACCATGGAGCGCCTCGAAGTCCACAGCAATGGCTCCGTC |
| | | GCCTACTTCAATGCTTCCCAGGTCACAGGGCCCAGCATCTACTCCTTCCACTGCGAGTATGTCAGCAGCC |
| | | TGAGCAAGAAGGGTAGTCTCCTCGTGGCCCGCACGCAGCCCTCTCCCTGGCAGATGATGCTTCAGGACTT |
| | | CCAGATCCAGGCTTTCAACGTAATGGGGGAGCAGTTCTCCTACGCCAGCGACTGTGCCAGCTTCTTCTCC |
| | | CCCGGCATCTGGATGGGGCTGCTCACCTCCCTGTTCATGCTCTTCATCTTCACCTATGGCCTGCACATGA |
| | | TCCTCAGCCTCAAGACCATGGATCGCTTTGATGACCACAAGGGCCCCACTATTTCTTTGACCCAGATTGT |
| | | GTGACCCTGTGCCAGTGGGGGGGTTGAGGGTGGGACGGTGTCCGTGTTGTTGCTTTCCCACCCTGCAGCG |
| | | CACTGGACTGAAGAGCTTCCCTCTTCCTACTGCAGCATGAACTGCAAGCTCCCCTCAGCCCATCTTGCTC |
| | | CCTCTTCAGCCCGCTGAGGAGCTTTCTTGGGCTGCCCCCATCTCTCCCAACAAGGTGTACATATTCTGCG |
| | | TAGATGCTAGACCAACCAGCTTCCCAGGGTTCGTCGCTGTGAGGCGTAAGGGACATGAATTCTAGGGTCT |
| | | CCTTTCTCCTTATTTATTCTTGTGGCTACATCATCCCTGGCTGTGGATAGTGCTTTTGTGTAGCAAATGC |
| | | TCCCTCCTTAAGGTTATAGGGCTCCCTGAGTTTGGGAGTGTGGAAGTACTACTTAACTGTCTGTCCTGCT |
| | | TGGCTGTCGTTATCGTTTTCTGGTGATGTTGTGCTAACAATAAGAAGTACACGGGTTTATTTCTGTGGCC |
| | | TGAGAAGGAAGGGACCTCCACGACAGGTGGGCTGGGTGCGATCGCCGGCTGTTTGGCATGTTCCCACCGG |
| | | GAGTGCCGGGCAGGAGCATGGGGTGCTTGGTTGTTTCCTTCCTAATAAAATAAACGCGGGTCGCCATGCA |
| | | AAAAAAAAA |
| 12 | major histo- | >gi\|52426773\|ref\|NM_019111.3\|Homo sapiens major histocompatibility |
| | compatibility | complex, class II, DR alpha (HLA-DRA), mRNA |
| | complex, class | ACATTCTCTTTTCTTTTATTCTTGTCTGTTCTGCCTCACTCCCGAGCTCTACTGACTCCCAACAGAGCGC |
| | II, DR alpha/// | CCAAGAAGAAAATGGCCATAAGTGGAGTCCCTGTGCTAGGATTTTTCATCATAGCTGTGCTGATGAGCGC |
| | major histo- | TCAGGAATCATGGGCTATCAAAGAAGAACATGTGATCATCCAGGCCGAGTTCTATCTGAATCCTGACCAA |
| | compatibility | TCAGGCGAGTTTATGTTTGACTTTGATGGTGATGAGATTTTCCATGTGGATATGGCAAAGAAGGAGACGG |
| | complex, class | TCTGGCGGCTTGAAGAATTTGGACGATTTGCCAGCTTTGAGGCTCAAGGTGCATTGGCCAACATAGCTGT |
| | II, DR alpha | GGACAAAGCCAACCTGGAAATCATGACAAAGCGCTCCAACTATACTCCGATCACCAATGTACCTCCAGAG |
| | | GTAACTGTGCTCACGAACAGCCCTGTGGAACTGAGAGAGCCCAACGTCCTCATCTGTTTCATAGACAAGT |
| | | TCACCCCACCAGTGGTCAATGTCACGTGGCTTCGAAATGGAAAACCTGTCACCACAGGAGTGTCAGAGAC |
| | | AGTCTTCCTGCCCAGGGAAGACCACCTTTTCCGCAAGTTCCACTATCTCCCCTTCCTGCCCTCAACTGAG |
| | | GACGTTTACGACTGCAGGGTGGAGCACTGGGGCTTGGATGAGCCTCTTCTCAAGCACTGGGAGTTTGATG |
| | | CTCCAAGCCCTCTCCCAGAGACTACAGAGAACGTGGTGTGTGCCCTGGGCCTGACTGTGGGTCTGGTGGG |
| | | CATCATTATTGGGACCATCTTCATCATCAAGGGATTGCGCAAAAGCAATGCAGCAGAACGCAGGGGGCCT |
| | | CTGTAAGGCACATGGAGGTGATGGTGTTTCTTAGAGAGAAGATCACTGAAGAAACTTCTGCTTTAATGGC |
| | | TTTACAAAGCTGGCAATATTACAATCCTTGACCTCAGTGAAAGCAGTCATCTTCAGCATTTTCCAGCCCT |
| | | ATAGCCACCCCAAGTGTGGATATGCCTCTTCGATTGCTCCGTACTCTAACATCTAGCTGGCTTCCCTGTC |
| | | TATTGCCTTTTCCTGTATCTATTTTCCTCTATTTCCTATCATTTTATTATCACCATGCAATGCCTCTGGA |
| | | ATAAAACATACAGGAGTCTGTCTCTGCTATGGAATGCCCCATGGGGCATCTCTTGTGTACTTATTGTTTA |
| | | AGGTTTCCTCAAACTGTGATTTTTCTGAACACAATAAACTATTTTGATGATCTTGGGTGGAAAAAAAAAA |
| | | AAAAAAA |
| Sequence | Major histo- | >gi\|24797068\|ref\|NM_002123.2\|Homo sapiens major histocompatibility |
| 13 | compatibility | complex, class II, DQ beta 1 (HLA-DQB1), mRNA |
| | complex, class | CAGATCCATCAGGTCCGAGCTGTGTTGACTACCACTTTTCCCTTCGTCTCAATTATGTCTTGGAAAAAGG |
| | II, DQ beta 1/// | CTTTGCGGATCCCCGGAGGCCTTCGGGCAGCAACTGTGACCTTGATGCTGTCGATGCTGAGCACCCCAGT |
| | Major histo- | GGCTGAGGGCAGAGACTCTCCCGAGGATTTCGTGTACCAGTTTAAGGGCATGTGCTACTTCACCAACGGG |
| | compatibility | ACAGAGCGCGTGCGTCTTGTGAGCAGAAGCATCTATAACCGAGAAGAGATCGTGCGCTTCGACAGCGACG |
| | complex, class | TGGGGGAGTTCCGGGCGGTGACGCTGCTGGGGCTGCCTGCCGCCGAGTACTGGAACAGCCAGAAGGACAT |
| | II, DQ beta 1 | CCTGGAGAGGAAACGGGCGGCGGTGGACAGGGTGTGCAGACACAACTACCAGTTGGAGCTCCGCACGACC |
| | | TTGCAGCGGCGAGTGGAGCCCACAGTGACCATCTCCCCATCCAGGACAGAGGCCCTCAACCACCACAACC |
| | | TGCTGGTCTGCTCGGTGACAGATTTCTATCCAGCCCAGATCAAAGTCCGGTGGTTTCGGAATGACCAGGA |
| | | GGAGACAGCTGGCGTTGTGTCCACCCCCCTTATTAGGAATGGTGACTGGACCTTCCAGATCCTGGTGATG |
| | | CTGGAAATGACTCCCCAGCGTGGAGACGTCTACACCTGCCACGTGGAGCACCCCAGCCTCCAGAGCCCCA |
| | | TCACCGTGGAGTGGCGGGCTCAATCTGAATCTGCCCAGAGCAAGATGCTGAGTGGCATTGGAGGCTTCGT |
| | | GCTGGGGCTGATCTTCCTCGGGCTGGGCCTTATCATCCATCACAGGAGTCAGAAAGGGCTCCTGCACTGA |
| | | CTCCTGAGACTATTTTAACTGGGATTGGTTATCACTTTTCTGTAACGCCTGCTTGTCCCTGCCCAGAATT |
| | | CCCAGCTGTCTGTGTCAGCCTGTCCCCCTGAGATCAGAGTCCTACAGTGGCTGTCACGCAGCCACCAGGT |
| | | CATCTCCTTTCATCCCCACCTTGAGGCGGATGGCTGTGACCCTACTTCCTGCACTGACCCACAGCCTCTG |
| | | CCTGTGCACGGCCAGCTGCATCTACTCAGGCCCCAAGGGGTTTCTGTTTCTATTCTCTCCTCAGACTGCT |
| | | CAAGAGAAGCACATGAAAACCATTACCTGACTTTAGAGCTTTTTTACATAATTAAACATGATCCTGAGTT |
| 14 | alcohol | >gi\|34577060\|ref\|NM_000668.3\|Homo sapiens alcohol dehydrogenase IB (class |
| | dehydrogenase | I), beta polypeptide (ADH1B), mRNA |
| | IB (class I), | ATGCACTCAAGCAGAGAAGAAATCCACAAAGACTCACAGTCTGCTGGTGGGCAGAGAAGACAGAAACGAC |
| | beta | ATGAGCACAGCAGGAAAAGTAATCAAATGCAAAGCAGCTGTGCTATGGGAGGTAAAGAAACCCTTTTCCA |
| | polypeptide | TTGAGGATGTGGAGGTTGCACCTCCTAAGGCTTATGAAGTTCGCATTAAGATGGTGGCTGTAGGAATCTG |
| | | TCACACAGATGACCACGTGGTTAGTGGCAACCTGGTGACCCCCCTTCCTGTGATTTTAGGCCATGAGGCA |
| | | GCCGGCATCGTGGAGAGTGTTGGAGAAGGGGTGACTACAGTCAAACCAGGTGATAAAGTCATCCCGCTCT |
| | | TTACTCCTCAGTGTGGAAAATGCAGAGTTTGTAAAAACCCGGAGAGCAACTACTGCTTGAAAAATGATCT |
| | | AGGCAATCCTCGGGGGACCCTGCAGGATGGCACCAGGAGGTTCACCTGCAGGGGGAAGCCCATTCACCAC |
| | | TTCCTTGGCACCAGCACCTTCTCCCAGTACACGGTGGTGGATGAGAATGCAGTGGCCAAAATTGATGCAG |
| | | CCTCGCCCCTGGAGAAAGTCTGCCTCATTGGCTGTGGATTCTCGACTGGTTATGGGTCTGCAGTTAACGT |
| | | TGCCAAGGTCACCCCAGGCTCTACCTGTGCTGTGTTTGGCCTGGGAGGGGTCGGCCTATCTGCTGTTATG |
| | | GGCTGTAAAGCAGCTGGAGCAGCCAGAATCATTGCGGTGGACATCAACAAGGACAAATTTGCAAAGGCCA |
| | | AAGAGTTGGGTGCCACTGAATGCATCAACCCTCAAGACTACAAGAAACCCATCCAGGAAGTGCTAAAGGA |
| | | AATGACTGATGGAGGTGTGGATTTTTCGTTTGAAGTCATCGGTCGGCTTGACACCATGATGGCTTCCCTG |
| | | TTATGTTGTCATGAGGCATGTGGCACAAGCGTCATCGTAGGGGTACCTCCTGCTTCCCAGAACCTCTCAA |
| | | TAAACCCTATGCTGCTACTGACTGGACGCACCTGGAAGGGGGCTGTTTATGGTGGCTTTAAGAGTAAAGA |
| | | AGGTATCCCAAAACTTGTGGCTGATTTTATGGCTAAGAAGTTTTCACTGGATGCGTTAATAACCCATGTT |
| | | TTACCTTTTGAAAAAATAAATGAAGGATTTGACCTGCTTCACTCTGGGAAAAGTATCCGTACCGTCCTGA |
| | | CGTTTTGAGGCAATAGAGATGCCTTCCCCTGTAGCAGTCTTCAGCCTCCTCTACCCTACAAGATCTGGAG |
| | | CAACAGCTAGGAAATATCATTAATTCAGCTCTTCAGAGATGTTATCAATAAATTACACATGGGGGCTTTC |
| | | CAAAGAAATGGAAATTGATGGGAAATTATTTTTCAGGAAAATTTAAAATTCAAGTGAGAAGTAAATAAAG |
| | | TGTTGAACATCAGCTGGGGAATTGAAGCCAACAAACCTTCCTTCTTAACCATTCTACTGTGTCACCTTTG |
| | | CCATTGAGGAAAAATATTCCTGTGACTTCTTGCATTTTTGGTATCTTCATAATCTTTAGTCATCGAATCC |
| | | CAGTGGAGGGGACCCTTTTACTTGCCCTGAACATACACATGCTGGGCCATTGTGATTGAAGTCTTCTAAC |
| | | TCTGTCTCAGTTTTCACTGTCGACATTTTCCTTTTTCTAATAAAAATGTACCAAATCCCTGGGGTAAAAG |
| | | CTAGGGTAAGGTAAAGGATAGACTCACATTTACAAGTAGTGAAGGTCCAAGAGTTCTAAATACAGGAAAT |
| | | TTCTTAGGAACTCAAATAAAATGCCCCACATTTTACTACAGTAAATGGCAGTGTTTTTATGACTTTTATA |
| | | CTATTTCTTTATGGTCGATATACAATTGATTTTTTAAAATAATAGCAGATTTCTTGCTTCATATGACAAA |
| | | GCCTCAATTACTAATTGTAAAAACTGAACTATTCCCAGAATCATGTTCAAAAAATCTGTAATTTTTGCTG |
| | | ATGAAAGTGCTTCATTGACTAAACAGTATTAGTTTGTGGCTATAAATGATTATTTAGATGATGACTGAAA |
| | | ATGTGTATAAAGTAATTAAAAGTAATATGGTGGCTTTAAGTGTAGAGATGGGATGGCAAATGCTGTGAAT |
| | | GCAGAATGTAAAATTGGTAACTAAGAAATGGCACAAACACCTTAAGCAATATATTTTCCTAGTAGATATA |
| | | TATATACACATACATATATACACATATACAAATGTATATTTTTGCAAAATTGTTTTCAATCTAGAACTTT |
| | | TCTATTAACTACCATGTCTTAAAATCAAGTCTATAATCCTAGCATTAGTTTAATATTTTGAATATGTAAA |
| | | GACCTGTGTTAATGCTTTGTTAATGCTTTTCCCACTCTCATTTGTTAATGCTTTCCCACTCTCAGGGGAA |
| | | GGATTTGCATTTTGAGCTTTATCTCTAAATGTGACATGCAAAGATTATTCCTGGTAAAGGAGGTAGCTGT |
| | | CTCCAAAAATGCTATTGTTGCAATATCTACATTCTATTTCATATTATGAAAGACCTTAGACATAAAGTAA |
| | | AATAGTTTATCATTTACTGTGTGATCTTCAGTAAGTCTCTCAGGCTCTCTGAGCTTGTTCATCCTTTGTT |
| | | TTGAAAAAATTACTCAACCAATCCATTACAGCTTAACCAAGATTAAATGGGATGATGTTAATGAAAGAGC |
| | | TTCGCC |
| 15 | T cell receptor | >gi\|36944\|emb\|X02592.1\|HSTCRAR Human mRNA for T-cell receptor alpha chain |
| | alpha constant/// | (TCR-alpha) |
| | T cell receptor | TTTTGAAACCCTTCAAAGGCAGAGACTTGTCCAGCCTAACCTGCCTGCTGCTCCTAGCTCCTGAGGCTCA |
| | alpha constant | GGGCCCTTGGCTTCTGTCCGCTCTGCTCAGGGCCCTCCAGCGTGGCCACTGCTCAGCCATGCTCCTGCTG |
| | | CTCGTCCCAGTGCTCGAGGTGATTTTTACCCTGGGAGGAACCAGAGCCCAGTCGGTGACCCAGCTTGGCA |
| | | GCCACGTCTCTGTCTCTGAAGGAGCCCTGGTTCTGCTGAGGTGCAACTACTCATCGTCTGTTCCACCATA |
| | | TCTCTTCTGGTATGTGCAATACCCCAACCAAGGACTCCAGCTTCTCCTGAAGTACACATCAGCGGCCACC |
| | | CTGGTTAAAGGCATCAACGGTTTTGAGGCTGAATTTAAGAAGAGTGAAACCTCCTTCCACCTGACGAAAC |
| | | CCTCAGCCCATATGAGCGACGCGGCTGAGTACTTCTGTGCTGTGAGTGATCTCGAACCGAACAGCAGTGC |
| | | TTCCAAGATAATCTTTGGATCAGGGACCAGACTCAGCATCCGGCCAAATATCCAGAACCCTGACCCTGCC |
| | | GTGTACCAGCTGAGAGACTCTAAATCCAGTGACAAGTCTGTCTGCCTATTCACCGATTTTGATTCTCAAA |
| | | CAAATGTGTCACAAAGTAAGGATTCTGATGTGTATATCACAGACAAAACTGTGCTAGACATGAGGTCTAT |
| | | GGACTTCAAGAGCAACAGTGCTGTGGCCTGGAGCAACAAATCTGACTTTGCATGTGCAAACGCCTTCAAC |
| | | AACAGCATTATTCCAGAAGACACCTTCTTCCCCAGCCCAGAAAGTTCCTGTGATGTCAAGCTGGTCGAGA |
| | | AAAGCTTTGAAACAGATACGAACCTAAACTTTCAAAACCTGTCAGTGATTGGGTTCCGAATCCTCCTCCT |
| | | GAAAGTGGCCGGGTTTAATCTGCTCATGACGCTGCGGCTGTGGTCCAGCTGAGATCTGCAAGATTGTAAG |
| | | ACAGCCTGTGCTCCCTCGCTCCTTCCTCTGCATTGCCCCTCTTCTCCCTCTCCAAACAGAGGGAACTCTC |
| | | CTACCCCCAAGGAGGTGAAAGCTGCTACCACCTCTGTGCCCCCCCGGTAATGCCACCAACTGGATCCTAC |
| | | CCGAATTTATGATTAAGATTGCTGAAGAGCTGCCAAACACTGCTGCCACCCCCTCTGTTCCCTTATTGCT |
| | | GCTTGTCACTGCCTGACATTCACGGCAGAGGCAAGGCTGCTGCAGCCTCCCCTGGCTGTGCACATTCCCT |
| | | CCTGCTCCCCAGAGACTGCCTCCGCCATCCCACAGATGATGGATCTTCAGTGGGTTCTCTTGGGCTCTAG |
| | | GTCCTGGAGAATGTTGTGAGGGGTTTATTTTTTTTTAATAGTGTTCATAAAGAAATACATAGTATTCTTC |
| | | TTCTCAAGACGTGGGGGGAAATTATCTCATTATCGAGGCCCTGCTATGCTGTGTGTCTGGGCGTGTTGTA |
| | | TGTCCTGCTGCCGATGCCTTCATTAAAATGATTTGGAA |
| 16 | CD69 antigen | >gi\|4502680\|ref\|NM_001781.1\|Homo sapiens CD69 molecule (CD69), mRNA |
| | (p60, early | AGACTCAACAAGAGCTCCAGCAAAGACTTTCACTGTAGCTTGACTTGACCTGAGATTAACTAGGGAATCT |
| | T-cell | TGAGAATAAAGATGAGCTCTGAAAATTGTTTCGTAGCAGAGAACAGCTCTTTGCATCCGGAGAGTGGACA |
| | activation | AGAAAATGATGCCACCAGTCCCCATTTCTCAACACGTCATGAAGGGTCCTTCCAAGTTCCTGTCCTGTGT |
| | antigen) | GCTGTAATGAATGTGGTCTTCATCACCATTTTAATCATAGCTCTCATTGCCTTATCAGTGGGCCAATACA |
| | | ATTGTCCAGGCCAATACACATTCTCAATGCCATCAGACAGCCATGTTTCTTCATGCTCTGAGGACTGGGT |
| | | TGGCTACCAGAGGAAATGCTACTTTATTTCTACTGTGAAGAGGAGCTGGACTTCAGCCCAAAATGCTTGT |
| | | TCTGAACATGGTGCTACTCTTGCTGTCATTGATTCTGAAAAGGACATGAACTTTCTAAAACGATACGCAG |
| | | GTAGAGAGGAACACTGGGTTGGACTGAAAAAGGAACCTGGTCACCCATGGAAGTGGTCAAATGGCAAAGA |
| | | ATTTAACAACTGGTTCAACGTTACAGGGTCTGACAAGTGTGTTTTTCTGAAAAACACAGAGGTCAGCAGC |
| | | ATGGAATGTGAGAAGAATTTATACTGGATATGTAACAAACCTTACAAATAATAAGGAAACATGTTCACTT |
| | | ATTGACTATTATAGAATGGAACTCAAGGAAATCTGTGTCAGTGGATGCTGCTCTGTGGTCCGAAGTCTTC |
| | | CATAGAGACTTTGTGAAAAAAAATTTTATAGTGTCTTGGGAATTTTCTTCCAAACAGAACTATGGAAAAA |
| | | AAGGAAGAAATTCCAGGAAAATCTGCACTGTGGGCTTTTATTGCCATGAGCTAGAAGCATCACAGGTTGA |
| | | CCAATAACCATGCCCAAGAATGAGAAGAATGACTATGCAACCTTTGGATGCACTTTATATTATTTTGAAT |
| | | CCAGAAATAATGAAATAACTAGGCGTGGACTTACTATTTATTGCTGAATGACTACCAACAGTGAGAGCCC |
| | | TTCATGCATTTGCACTACTGGAAGGAGTTAGATGTTGGTACTAGATACTGAATGTAAACAAAGGAATTAT |
| | | GGCTGGTAACATAGGTTTTTAGTCTAATTGAATCCCTTAAACTCAGGGAGCATTTATAAATGGACAAATG |
| | | CTTATGAAACTAAGATTTGTAATATTTCTCTCTTTTTAGAGAAATTTGCCAATTTACTTTGTTATTTTTC |
| | | CCCAAAAAGAATGGGATGATCGTGTATTTATTTTTTTACTTCCTCAGCTGTAGACAGGTCCTTTTCGATG |
| | | GTACATATTTCTTTGCCTTTATAATCTTTTATACAGTGTCTTACAGAGAAAAGACATAAGCAAAGACTAT |
| | | GAGGAATATTTGCAAGACATAGAATAGTGTTGGAAAATGTGCAATATGTGATGTGGCAAATCTCTATTAG |
| | | GAAATATTCTGTAATCTTCAGACCTAGAATAATACTAGTCTTATAATAGGTTTGTGACTTTCCTAAATCA |
| | | ATTCTATTACGTGCAATACTTCAATACTTCATTTAAAATATTTTTATGTGCAATAAAATGTATTTGTTTG |
| | | TATTTTGTGTTCAGTACAATTATAAGCTGTTTTTATATATGTGAAATAAAAGTAGAATAAACACAAAAAA |
| | | AAAAAAAAAAAAAAAAAAAAAA |
| 17 | protein kinase | >gi\|48255887\|ref\|NM_006257.2\|Homo sapiens protein kinase C, theta |
| | C, theta | (PRKCQ), mRNA |
| | | AGTCCCCGCGCAGTCCCCGCGCAGTCCCAGCGCCACCGGGCAGCAGCGGCGCCGTGCTCGCTCCAGGGCG |
| | | CAACCATGTCGCCATTTCTTCGGATTGGCTTGTCCAACTTTGACTGCGGGTCCTGCCAGTCTTGTCAGGG |
| | | CGAGGCTGTTAACCCTTACTGTGCTGTGCTCGTCAAAGAGTATGTCGAATCAGAGAACGGGCAGATGTAT |
| | | ATCCAGAAAAAGCCTACCATGTACCCACCCTGGGACAGCACTTTTGATGCCCATATCAACAAGGGAAGAG |
| | | TCATGCAGATCATTGTGAAAGGCAAAAACGTGGACCTCATCTCTGAAACCACCGTGGAGCTCTACTCGCT |
| | | GGCTGAGAGGTGCAGGAAGAACAACGGGAAGACAGAAATATGGTTAGAGCTGAAACCTCAAGGCCGAATG |
| | | CTAATGAATGCAAGATACTTTCTGGAAATGAGTGACACAAAGGACATGAATGAATTTGAGACGGAAGGCT |
| | | TCTTTGCTTTGCATCAGCGCCGGGGTGCCATCAAGCAGGCAAAGGTCCACCACGTCAAGTGCCACGAGTT |
| | | CACTGCCACCTTCTTCCCACAGCCCACATTTTGCTCTGTCTGCCACGAGTTTGTCTGGGGCCTGAACAAA |
| | | CAGGGCTACCAGTGCCGACAATGCAATGCAGCAATTCACAAGAAGTGTATTGATAAAGTTATAGCAAAGT |
| | | GCACAGGATCAGCTATCAATAGCCGAGAAACCATGTTCCACAAGGAGAGATTCAAAATTGACATGCCACA |
| | | CAGATTTAAAGTCTACAATTACAAGAGCCCGACCTTCTGTGAACACTGTGGGACCCTGCTGTGGGGACTG |
| | | GCACGGCAAGGACTCAAGTGTGATGCATGTGGCATGAATGTGCATCATAGATGCCAGACAAAGGTGGCCA |
| | | ACCTTTGTGGCATAAACCAGAAGCTAATGGCTGAAGCGCTGGCCATGATTGAGAGCACTCAACAGGCTCG |
| | | CTGCTTAAGAGATACTGAACAGATCTTCAGAGAAGGTCCGGTTGAAATTGGTCTCCCATGCTCCATCAAA |
| | | AATGAAGCAAGGCCGCCATGTTTACCGACACCGGGAAAAAGAGAGCCTCAGGGCATTTCCTGGGAGTCTC |
| | | CGTTGGATGAGGTGGATAAAATGTGCCATCTTCCAGAACCTGAACTGAACAAAGAAAGACCATCTCTGCA |
| | | GATTAAACTAAAAATTGAGGATTTTATCTTGCACAAAATGTTGGGGAAAGGAAGTTTTGGCAAGGTCTTC |
| | | CTGGCAGAATTCAAGAAAACCAATCAATTTTTCGCAATAAAGGCCTTAAAGAAAGATGTGGTCTTGATGG |
| | | ACGATGATGTTGAGTGCACGATGGTAGAGAAGAGAGTTCTTTCCTTGGCCTGGGAGCATCCGTTTCTGAC |
| | | GCACATGTTTTGTACATTCCAGACCAAGGAAAACCTCTTTTTTGTGATGGAGTACCTCAACGGAGGGGAC |
| | | TTAATGTACCACATCCAAAGCTGCCACAAGTTCGACCTTTCCAGAGCGACGTTTTATGCTGCTGAAATCA |
| | | TTCTTGGTCTGCAGTTCCTTCATTCCAAAGGAATAGTCTACAGGGACCTGAAGCTAGATAACATCCTGTT |
| | | AGACAAAGATGGACATATCAAGATCGCGGATTTTGGAATGTGCAAGGAGAACATGTTAGGAGATGCCAAG |
| | | ACGAATACCTTCTGTGGGACACCTGACTACATCGCCCCAGAGATCTTGCTGGGTCAGAAATACAACCACT |
| | | CTGTGGACTGGTGGTCCTTCGGGGTTCTCCTTTATGAAATGCTGATTGGTCAGTCGCCTTTCCACGGGCA |
| | | GGATGAGGAGGAGCTCTTCCACTCCATCCGCATGGACAATCCCTTTTACCCACGGTGGCTGGAGAAGGAA |
| | | GCAAAGGACCTTCTGGTGAAGCTCTTCGTGCGAGAACCTGAGAAGAGGCTGGGCGTGAGGGGAGACATCC |
| | | GCCAGCACCCTTTGTTTCGGGAGATCAACTGGGAGGAACTTGAACGGAAGGAGATTGACCCACCGTTCCG |
| | | GCCGAAAGTGAAATCACCATTTGACTGCAGCAATTTCGACAAAGAATTCTTAAACGAGAAGCCCCGGCTG |
| | | TCATTTGCCGACAGAGCACTGATCAACAGCATGGACCAGAATATGTTCAGGAACTTTTCCTTCATGAACC |
| | | CCGGGATGGAGCGGCTGATATCCTGAATCTTGCCCCTCCAGAGACAGGAAAGAATTTGCCTTCTCCCTGG |
| | | GAACTGGTTCAAGAGACACTGCTTGGGTTCCTTTTTCAACTTGGAAAAAGAAAGAAACACTCAACAATAA |
| | | AGACTGAGACCCGTTCGCCCCCATGTGACTTTTATCTGTAGCAGAAACCAAGTCTACTTCACTAATGACG |
| | | ATGCCGTGTGTCTCGTCTCCTGACATGTCTCACAGACGCTCCTGAAGTTAGGTCATTACTAACCATAGTT |
| | | ATTTACTTGAAAGATGGGTCTCCGCACTTGGAAAGGTTTCAAGACTTGATACTGCAATAAATTATGGCTC |
| | | TTCACCTGGGCGCCAACTGCTGATCAATGAAATGCTTGTTGAATCAGGGGCAAACGGAGTACAGACGTCT |
| | | CAAGACTGAAACGGCCCCATTGCCTGGTCTAGTAGCGGATCTCACTCAGCCGCAGACAAGTAATCACTAA |
| | | CCCGTTTTATTCTATTCCTATCTGTGGATGTGTAAATGGCTGGGGGGCCAGCCCTGGATAGGTTTTTATG |
| | | GGAATTCTTTACAATAAACATAGCTTGTAACTTGAGATCTACAAATCCATTCATCCTGATTGGGCATGAA |
| | | ATCCATGGTCAAGAGGACAAGTGGAAAGTGAGAGGGAAGGTTTGCTAGACACCTTCGCTTGTTATCTTGT |
| | | CAAGATAGAAAAGATAGTATCATTTCACCCTTGCCAGTAAAAACCTTTCCATCCACCCATTCTCAGCAGA |
| | | CTCCAGTATTGGCACAGTCACTCACTGCCATTCTCACACTATAACAAGAAAAGAAATGAAGTGCATAAGT |
| | | CTCCTGGGAAAAGAACCTTAACCCCTTCTCGTGCCATGACTGGTGATTTCATGACTCATAAGCCCCTCCG |
| | | TAGGCATCATTCAAGATCAATGGCCCATGCATGCTGTTTGCAGCAGTCAATTGAGTTGAATTAGAATTCC |
| | | AACCATACATTTTAAAGGTATTTGTGCTGTGTGTATATTTTGATAAAATGTTGTGACTTCATGGCAAACA |
| | | GGTGGATGTGTAAAAATGGAATAAAAAAAAAAAAAGAGTCAAAAAAAAAAAAA |
| 18 | T cell receptor | >gi\|339011\|gb\|M15564.1\|HUMTCBYZ Human T-cell receptor rearranged beta- |
| | beta variable | chain V-region (V-D-J) mRNA, complete cds |
| | 19///T cell | GTAAAGCTCCCATCCTGCCCTGACCCTGCCATGGGCACCAGCCTCCTCTGCTGGATGGCCCTGTGTCTCC |
| | receptor beta | TGGGGGCAGATCACGCAGATACTCGAGTCTCCCAGAACCCCAGACACAACATCACAAAGAGGGGACAGAA |
| | constant 1 | TGTAACTTTCAGGTGTGATCCAATTTCTGAACACAACCGCCTTTATTGGTACCGACAGACCCTGGGGCAG |
| | | GGCCCAGAGTTTCTGACTTACTTCCAGAATGAAGCTCAACTAGAAAAATCAAGGCTGCTCAGTGATCGGT |
| | | TCTCTGCAGAGAGGCCTAAGGGATCTTTCTCCACCTTGGAGATCCAGCGCACAGAGCAGGGGGACTCGGC |
| | | CATGTATCTCTGTGCCAGCAGCTTAGCAGGGTTGAATCAGCCCCAGCATTTTGGTGATGGGACTCGACTC |
| | | TCCATCCTAGAGGACCTGAACAAGGTGTTCCCACCCGAGGTCGCTGTGTTTGAGCCATCAGAAGCAGAGA |
| | | TCTCCCACACCCAAAAGGCCACACTGGTGTGCCTGGCCACAGGTATCTTCCCTGACCACGTGGAGCTGAG |
| | | CTGGTGGGTGAATGGGAAGGAGGTGCACAGTGGGGTCAGCACGGACCCGCAGCCCCTCAAGGAGCAGCCC |
| | | GCCCTCAATGACTCCAGATACTGCCTGAGCAGCCGCCTGAGGGTCTCGGCCACCTTCTGGCAGAACCCCC |
| | | GCAACCACTTCCGCTGTCAAGTCCAGTTCTACGGGCTCTCGGAGAATGACGAGTGGACCCAGGATAGGGC |
| | | CAAACCCGTCACCCAGATCGTCAGCGCCGAGGCCTGGGGTAGAGCAGACTGTGGCTTTACCTCGGTGTCC |
| | | TACCAGCAAGGGGTCCTGTCTGCCACCATCCTCTATGAGATCCTGCTAGGGAAGGCCACCATGTATGCTG |
| | | TGCTGGTCAGCGCCCTTGTGTTGATGGCCATGGTCAAGAGAAAGGATTTCTGAAGGCAGCCCTGGAAGTG |
| | | GAGTTAGGAGCTTCTAACCCGTCATGGTTTCAATACACATTCTTCTTTTGCCAGCGCTTCTGAAGAGCTG |
| | | CTCTCACCTCTCTGCATCCCAATA |
| 19 | T cell receptor | >gi\|338765\|gb\|M15565.1\|HUMTCAYE Human T-cell receptor rearranged alpha- |
| | alpha locus///T | chain V-region (V-D-J) mRNA, complete cds |
| | cell receptor | TCCAAAATGAAGGGTCTGTGGAAGGACATGAATAAAGCACAGGAGGTTGAAGTCAGATTTGCAGCTTTCT |
| | delta variable | AGGCAGGAGACAAGACAATCTGCATCTTCACAGGAGGGATGGCCATGCTCCTGGGGGCATCAGTGCTGAT |
| | 2///T cell | TCTGTGGCTTCAGCCAGACTGGGTAAACAGTCAACAGAAGAATGATGACCAGCAAGTTAAGCAAAATTCA |
| | receptor alpha | CCATCCCTGAGCGTCCAGGAAGGAAGAATTTCTATTCTGAACTGTGACTATACTAACAGCATGTTTGATT |
| | variable 20///T | ATTTCCTATGGTACAAAAAATACCCTGCTGAAGGTCCTACATTCCTGATATCTATAAGTTCCATTAAGGA |
| | cell receptor | TAAAAATGAAGATGGAAGATTCACTGTCTTCTTAAACAAAAGTGCCAAGCACCTCTCTCTGCACATTGTG |
| | alpha joining | CCCTCCCAGCCTGGAGACTCTGCAGTGTACTTCTGTGCAGCAAACGGGGCCGGCACTGCCAGTAAACTCA |
| | 17///T cell | CCTTTGGGACTGGAACAAGACTTCAGGTCACGCTCGATATCCAGAACCCTGACCCTGCCGTGTACCAGCT |
| | receptor alpha | GAGAGACTCTAAATCCAGTGACAAGTCTGTCTGCCTATTCACCGATTTTGATTCTCAAACAAATGTGTCA |
| | constant | CAAAGTAAGGATTCTGATGTGTATATCACAGACAAAACTGTGCTAGACATGAGGTCTATGGACTTCAAGA |
| | | GCAACAGTGCTGTGGCCTGGAGCAACAAATCTGACTTTGCATGTGCAAACGCCTTCAACAACAGCATTAT |
| | | TCCAGAAGACACCTTCTTCCCCAGCCCAGAAAGTTCCTGTGATGTCAAGCTGGTCGAGAAAAGCTTTGAA |
| | | ACAGATACGAACCTAAACTTTCAAAACCTGTCAGTGATTGGGTTCCGAATCCTCCTCCTGAAAGTGGCCG |
| | | GGTTTAATCTGCTCATGACGCTGCGGCTGTGGTCCAGCTGAGATCTGCAAGATTGTAAGACAGCCTGTGC |
| | | TCCCTCGCTCCTTCCTCTGCATTGCCCCTCTTCTCCCTCTCCAAACAGAGGGAACTCTCCCACCCCCAAG |
| | | GAGGTGAAAGCTGCTACCACCCTGTGTGCC |
| 20 | T cell receptor | >gi\|339406\|gb\|M30894.1\|HUMTCRGAD Human T-cell receptor Ti rearranged |
| | gamma constant 2 | gamma-chain mRNA V-J-C region, complete cds |
| | | GAATCAGGAAGACCAGCTCCTCCTACTGTCTTCTGTGTTACGGGATCAGCGTTCCTTGTTGAGTGGGACC |
| | | TGAGTTTTGAGAGGGTCTTCTGCTCCTCTTGGTCTGGTCCCTTACTTCCAAGAGCCCCAGAGAGGAAGGC |
| | | ATGCTGTTGGCTCTAGCTCTGCTTCTAGCTTTCCTGCCTCCTGCCAGTCAGAAATCTTCCAACTTGGAAG |
| | | GGAGAACAAAGTCAGTCACCAGGCCAACTGGGTCATCAGCTGTAATCACTTGTGATCTTCCTGTAGAAAA |
| | | TGCCGTCTACACCCACTGGTACCTACACCAGGAGGGGAAGGCCCCACAGCGTCTTCTGTACTATGACTCC |
| | | TACAACTCCAGGGTTGTGTTGGAATCAGGAATCAGTCGAGAAAAGTATCATACTTATGCAAGCACAGGGA |
| | | AGAGCCTTAAATTTATACTGGAAAATCTAATTGAACGTGACTCTGGGGTCTATTACTGTGCCACCTGGAA |
| | | GGATTATTATAAGAAACTCTTTGGCAGTGGAACAACACTTGTTGTCACAGATAAACAACTTGATGCAGAT |
| | | GTTTCCCCCAAGCCCACTATTTTTCTTCCTTCGATTGCTGAAACAAAACTCCAGAAGGCTGGAACATATC |
| | | TTTGTCTTCTTGAGAAATTTTTCCCAGATATTATTAAGATACATTGGCAAGAAAAGAAGAGCAACACGAT |
| | | TCTGGGATCCCAGGAGGGGAACACCATGAAGACTAACGACACATACATGAAATTTAGCTGGTTAACGGTG |
| | | CCAGAAGAGTCACTGGACAAAGAACACAGATGTATCGTCAGACATGAGAATAATAAAAACGGAATTGATC |
| | | AAGAAATTATCTTTCCTCCAATAAAGACAGATGTCACCACAGTGGATCCCAAAGACAGTTATTCAAAAGA |
| | | TGCAAATGATGTCACCACAGTGGATCCCAAATACAATTATTCAAAGGATGCAAATGATGTCATCACAATG |
| | | GATCCCAAAGACAATTGGTCAAAAGATGCAAATGATACACTACTGCTGCAGCTCACAAACACCTCTGCAT |
| | | ATTACATGTACCTCCTCCTGCTCCTCAAGAGTGTGGTCTATTTTGCCATCATCACCTGCTGTCTGCTTGG |
| | | AAGAACGGCTTTCTGCTGCAATGGAGAGAAATCATAACAGACGGTGGCACAAGGAGGCCATCTTTTCCTC |
| | | ATCGGTTATTGTCCCTAGAAGCGTCTTCTGAGGATCTAGTTGGGCTTTCTTTCTGGGTTTGGGCCATTTC |
| | | AGTTCTCATGTGTGtACTATTCTATCATTATTGTATAATGGTTTTCAAACCAGTGGGCACACAGAGAACC |
| | | TCAGTCTGTAATAACAATGAGGAATAGCCATGGCGATCTCCAGCACCAATCTCTCCATGTTTTCCACAGC |
| | | TCCTCCAGCCAACCCAAATAGCGCCTGCTATAGTGTAGACAGCCTGCGGCTTCTAGCCTTGTCCCTCTCT |
| | | TAGTGTTCTTTAATCAGATAACTGCCTGGAAGCCTTTCATTTTACACGCCCTGAAGCAGTCTTCTTTGCT |
| | | AGTTGAATTATGTGGTGTGTTTTTCCGTAATAAGCAAAATAAATTT |
| 21 | T cell receptor | >gi\|3002924\|gb\|AF043179.1\|AF043179 Homo sapiens T cell receptor beta |
| | beta variable | chain (TCRBV13S1-TCRBJ2S1) mRNA, complete cds |
| | 21-1///T cell | ATGAGCATCGGCCTCCTGTGCTGTGCAGCCTTGTCTCTCCTGTGGGCAGGTCCAGTGAATGCTGGTGTCA |
| | receptor beta | CTCAGACCCCAAAATTCCAGGTCCTGAAGACAGGACAGAGCATGACACTGCAGTGTGCCCAGGATATGAA |
| | variable 19/// | CCATGAATACATGTCCTGGTATCGACAAGACCCAGGCATGGGGCTGAGGCTGATTCATTACTCAGTTGGT |
| | T cell receptor | GCTGGTATCACTGACCAAGGAGAAGTCCCCAATGGCTACAATGTCTCCAGATCAACCACAGAGGATTTCC |
| | beta variable | CGCTCAGGCTGCTGTCGGCTGCTCCCTCCCAGACATCTGTGTACTTCTGTGCCAGCAGTTTCCCCCGGCA |
| | 5-4///T cell | GCCGTCCTACAATGAGCAGTTCTTCGGGCCAGGGACACGGCTCACCGTGCTAGAGGACCTGAAAAACGTG |
| | receptor beta | TTCCCACCCGAGGTCGCTGTGTTTGAGCCATCAGAAGCAGAGATCTCCCACACCCAAAAGGCCACACTGG |
| | variable 3-1/// | TGTGCCTGGCCACAGGTTTCTACCCCGACCACGTGGAGCTGAGCTGGTGGGTGAATGGGAAGGAGGTGCA |
| | T cell receptor | CAGTGGGGTCAGCACAGACCCGCAGCCCCTCAAGGAGCAGCCCGCCCTCAATGACTCCAGATACTGCCTG |
| | beta constant 1 | AGCAGCCGCCTGAGGGTCTCGGCCACCTTCTGGCAGAACCCCCGCAACCACTTCCGCTGTCAAGTCCAGT |
| | | TCTACGGGCTCTCGGAGAATGACGAGTGGACCCAGGATAGGGCCAAACCTGTCACCCAGATCGTCAGCGC |
| | | CGAGGCCTGGGGTAGAGCAGACTGTGGCTTCACCTCCGAGTCTTACCAGCAAGGGGTCCTGTCTGCCACC |
| | | ATCCTCTATGAGATCTTGCTAGGGAAGGCCACCTTGTATGCTGTGCTGGTCAGTGCCCTCGTGCTGATGG |
| | | CCATGGTCAAGAGAAAGGATTCCAGAGGCTGA |
| 22 | T cell receptor | >gi\|1100165\|gb\|L34703.1\|HUMTCRAZ Homo sapiens T-cell receptor alpha chain |
| | alpha locus | (TCRA) mRNA (HLA-A1, 24; B7, 8; DR 1, 3), complete cds |
| | | ATGGCATGCCCTGGCTTCCTGTGGGCACTTGTGATCTCCACCTGTCTTGAATTTAGCATGGCTCAGACAG |
| | | TCACTCAGTCTCAACCAGAGATGTCTGTGCAGGAGGCAGAGACCGTGACCCTGAGCTGCACATATGACAC |
| | | CAGTGAGAGTGATTATTATTTATTCTGGTACAAGCAGCCTCCCAGCAGGCAGATGATTCTCGTTATTCGC |
| | | CAAGAAGCTTATAAGCAACAGAATGCAACAGAGAATCGTTTCTCTGTGAACTTCCAGAAAGCAGCCAAAT |
| | | CCTTCAGTCTCAAGATCTCAGACTCACAGCTGGGGGATGCCGCGATGTATTTCTGTGCTTATAGGAGTGC |
| | | ATACTCTGGGGCTGGGAGTTACCAACTCACTTTCGGGAAGGGGACCAAACTCTCGGTCATACCAAATATC |
| | | CAGAACCCTGACCCTGCCGTGTACCAGCTGAGAGACTCTAAATCCAGTGACAAGTCTGTCTGCCTATTCA |
| | | CCGATTTTGATTCTCAAACAAATGTGTCACAAAGTAAGGATTCTGATGTGTATATCACAGACAAAACTGT |
| | | GCTAGACATGAGGTCTATGGACTTCAAGAGCAACAGTGCTGTGGCCTGGAGCAACAAATCTGACTTTGCA |
| | | TGTGCAAACGCCTTCAACAACAGCATTATTCCAGAAGACACCTTCTTCCCCAGCCCAGAAAGTTCCTGTG |
| | | ATGTCAAGCTGGTCGAGAAAAGCTTTGAAACAGATACGAACCTAAACTTTCAAAACCTGTCAGTGATTGG |
| | | GTTCCGAATCCTCCTCCTGAAAGTGGCCGGGTTTAATCTGCTCATGACGCTGCGGTTGTGGTCCAGCTGA |
| 23 | T cell receptor | >gi\|46184507\|gb\|AL559122.3\|AL559122 AL559122 Homo sapiens T CELLS (JURKAT |
| | beta variable | CELL LINE) COT 10-NORMALIZED Homo sapiens cDNA clone CS0DJ014YE01 5-PRIME, |
| | 19///T cell | mRNA sequence |
| | receptor beta | TGCCTGGCCACAGGCTTCTTCCCCGACCACGTGGAGCTGAGCTGGTGGGTGAATGGGAAGGAGGTGCACA |
| | variable 19/// | ATGGGGTCAACACAGACCCGCAGCCCCTCAAAGAACAGCCCGCCCTCAATGACTCCAAATAMTGCCTGAG |
| | T cell receptor | CAGCCGCCTGAGGGTCTCGGCCACCTTCTGGCAGAACCCCCGCAACCACTTCCGCTGTCAAGTCCAGTTC |
| | beta constant | TACGGGCTCTCGGAGAATGACGAGTGGACCCAGGATAGGGCCAAACCCGTCACCCAAATCGTCAGCGCCG |
| | 1///T cell | AGGCCTGGGGTAPAGCARAMTGTGGCTTTACCTCGGTGTCCTACCAACAAGGGGTCCTGTCTGCCACCAT |
| | receptor beta | CCTCTATGARATCCTGCTAGGGAAGGCCACCCTGTATGCTGTGCTGGTCAGCGCCCTTGTGTTGATGGCC |
| | constant 1 | ATGGTCAAGAGAAAGGATTTCTGAAGGCAGCCCTGGAAGTGGAATTAAGAACTTCTAACCCGTCATGGTT |
| | | TCAATACACATTCTTCTTTTGCCAGCGCTTCTGAAGAGCTGCTCTCACCTCTCTGCATCCCAATAGATAT |
| | | CCCCCTATGTGCATGCACACCTGCACACTCACGGCTGAAATCTCCCTAACCCAGGCGGACCTTAGCATGC |
| | | CTAAGTGACTAAACCAATAAAAATGTTCTGGTCTGGCCTGA |
| 24 | CD3D antigen, | >gi\|98985799\|ref\|NM_000732.41\|Homo sapiens CD3d molecule, delta (CD3-TCR |
| | delta polypep- | complex) (CD3D), transcript variant 1, mRNA |
| | tide (TiT3) | AGAGAAGCAGACATCTTCTAGTTCCTCCCCCACTCTCCTCTTTCCGGTACCTGTGAGTCAGCTAGGGGAG |
| | complex) | GGCAGCTCTCACCCAGGCTGATAGTTCGGTGACCTGGCTTTATCTACTGGATGAGTTCCGCTGGGAGATG |
| | | GAACATAGCACGTTTCTCTCTGGCCTGGTACTGGCTACCCTTCTCTCGCAAGTGAGCCCCTTCAAGATAC |
| | | CTATAGAGGAACTTGAGGACAGAGTGTTTGTGAATTGCAATACCAGCATCACATGGGTAGAGGGAACGGT |
| | | GGGAACACTGCTCTCAGACATTACAAGACTGGACCTGGGAAAACGCATCCTGGACCCACGAGGAATATAT |
| | | AGGTGTAATGGGACAGATATATACAAGGACAAAGAATCTACCGTGCAAGTTCATTATCGAATGTGCCAGA |
| | | GCTGTGTGGAGCTGGATCCAGCCACCGTGGCTGGCATCATTGTCACTGATGTCATTGCCACTCTGCTCCT |
| | | TGCTTTGGGAGTCTTCTGCTTTGCTGGACATGAGACTGGAAGGCTGTCTGGGGCTGCCGACACACAAGCT |
| | | CTGTTGAGGAATGACCAGGTCTATCAGCCCCTCCGAGATCGAGATGATGCTCAGTACAGCCACCTTGGAG |
| | | GAAACTGGGCTCGGAACAAGTGAACCTGAGACTGGTGGCTTCTAGAAGCAGCCATTACCAACTGTACCTT |
| | | CCCTTCTTGCTCAGCCAATAAATATATCCTCTTTCACTCAGAAAAAAAAAAAAAAAAAAAAAAAAAAAAA |
| | | A |
| 25 | T cell receptor | >gi\|339168\|gb\|M13231.1\|HUMTCGXH Human T-cell receptor aberrantly |
| | gamma constant | rearranged gamma-chain mRNA from cell line HPB-MLT |
| | 2///T cell | GGCATGCGGTGGGCCCTACTGGTGCTTCTAGCTTTCCTGTCTCCTGCCAGTCAGAAATCTTCCAACTTGG |
| | receptor gamma | AAGGGAGAACGAAGTCAGTCACCAGGCAGACTGGGTCATCTGCTGAAATCACTTGCGATCTTACTGTAAC |
| | variable 9/// | AAATACCTTCTACATCCACTGGTACCTACACCAGGAGGGGAAGGCCCCACAGCGTCTTCTGTACTATGAC |
| | similar to T- | GTCTCCACTGCAAGGGATGTGTTGGAATCAGGACTCAGTCCAGGAAAGTATTATACTCATACACCCAGGA |
| | cell receptor | GGTGGAGCTGGATATTGAGACTGCAAAATCTAATTGAAAATGATTCTGGGGTCTATTACTGTGCCACCTG |
| | gamma chain C | GGACAGGCAAAAATTATTATAAGAAACTCTTTGGCAGTGGAACAACACTTGTTGTCACAGATAAACAACT |
| | region PT- | TGATGCAGATGTTTCCCCCAAGCCCACTATTTTTCTTCCTTCAATTGCTGAAACAAAACTCCAGAAGGCT |
| | gamma-½/// | GGAACATACCTTTGTCTTCTTGAGAAATTTTTCCCAGATATTATTAAGATACATTCGCAAGAAAAGAAGA |
| | similar to T- | GCAACACGATTCTGGGATCCCAGGAGGGGAACACCATGAAGACTAACGACACATACATGAAATTTAGCTG |
| | cell receptor | GTTAACGGTGCCAGAAGAGTCACTGGACAAAGAACACAGATGTATCGTCAGACATGAGAATAATAAAAAC |
| | gamma chain V | GGAATTGATCAAGAAATTATCTTTCCTCCAATAAAGACAGATGTCACCACAGTGGATCCCAAAGACAGTT |
| | region PT- | ATTCAAAAGATGCAAATGATGTCATCACAATGGATCCCAAAGACAATTGGTCAAAAGATGCAAATGATAC |
| | gamma-½ | ACTACTGCTGCAGCTCACAAACACCTCTGCATATTACATGTACCTCCTCCTGCTCCTCAAGAGTGTGGTC |
| | precursor/// | TATTTTGCCATCATCACCTGCTGTCTGCTTGGAAGAACGGCTTTGTGCTGCAATGGAGAGAAATCATAAC |
| | TCR gamma | AGACGGTGGCACAAGGAGGCCATCTTTTCCTCATCGGTTATTGTCCCTAGAAGCGTCTTCTGAGGATCTA |
| | alternate | GTTGGGCTTTCTTTCTGGGTTTGGGCCATTTCAGTTCTCATGTGTGTAGTATTCTATCATTATTGTATAA |
| | reading frame | TGGTTTTCAAACCAGTGGGCACACAGAGAACCTCAGTCTGTAATAACAATGAGGAATAGCCATGGCGATC |
| | protein | TCCAGCACCAATCTCTCCATGTTTTCCACAGCTCCTCCAGCCAACCCAAATAGCGCCTGCTATAGTGTAG |
| | | ACAGCCTGCGGCTTCTAGCCTTGTCCCTCTCTTAGTGTTCTTTAATCAGATAACTGCCTCGAAGCCTTTC |
| | | ATTTTACACGCCCTGAAGCAGTCTTCTTTGCTAGTTGAATTATGTGGTGTGTTTTTCCGTAATAAGCAAA |
| | | ATAAATTTAAAAAAATGAAAA |
| 26 | pyrin and HIN | >gi\|10437303\|dbj\|AK024890.1\|Homo sapiens cDNA: FLJ21237 fis, clone |
| | domain family, | COL01114 |
| | member 1 | GTCAAAGGAATAATCCCATCTAAAAAGACGAAACAGAAAGAAGTGTATCCTGCTACACCTGCATGCACCC |
| | | CAAGCAACCGTCTCACAGCTAAAGGAGCAGAGGAGACTCTTGGACCTCAGGTAAGCTTCAGGAAGAGGAG |
| | | CAGGCTTCAAGTCTCACAGTGGAAGCTCTGCTGTGGCTGTTCCACTCAATCTGTCCAGCAGGCAGTTATT |
| | | TCTTCATATGTTTCCCATCAAGTTTCAGATTTATCAAATTACATAATAATTGATCATCTTTCTGCAAGGC |
| | | AACAAGTTAAACGCTTTAGTAAACATAATGTAAATATACATAAAATAAATATAATATTTTCATCTCCAAT |
| | | AGAGAAGGATGTTAACTTGAGAGTCAGATAAAAAAACGTTTGCCTATGTTTACAAAAGCCTAGTTTCTTA |
| | | ACTGCAAGTCAGCATATCCCAAAACACAAGTAATTAAGGAATGATGTGTGTTACTTTCTCTGCTCCCTTT |
| | | TTAAAAATGAAACCATCTATGCCATGTTCTTTCAATTGGCCTGGGGATGTACTTAAGTTTCCAAGAAAAA |
| | | CAATTTATATACAATAAATATATTACCTTGTAATGAAAATGTGCTCTGCTTCATTTGACACTGAAAGTAA |
| | | TTAACAAGAAAAATAAACTACTTGTAGAAAAGAAAAAAACCATCTGAAGAAGAGACTGGAACCAAAAGGA |
| | | GTAAGATGTCCAAAGAGCAGACTCGGCCTTCCTGCTCTGCAGGAGCCAGCACGTCCACAGCCATGGGCCG |
| | | TTCCCCACCTCCCCAGACCTCATCATCAGCTCCACCCAACACTTCCTCAACTGAGGTACACTCTTCCTGG |
| | | TCCCCTTTTGATTCATTTTCTTCAACCCAAAATGTAGGAATCTGATTTCATCTTCTACTGAAAAATGACA |
| | | TCAATCATCAGCCAGTAAATCAAATGTATAGACTGAGAATTAACTGCATTTTAATCTTTTGCTTCCACAG |
| | | GCATATTTGATGAACTTGACATTATCTCTGACTGCAGGAAGTTTTCTGTCCTGTGCTGTTTGGGGAAGAG |
| | | ACAGAGAACTGCGGAATCTGGAACTTTCAGCAACAGACTCACTGTCTACTGCCCCCATCTATTATACACC |
| | | CATTCCCTTTGCTCACTAATTTGTTCAAGTTTCTCTGACATACACCATGCTCCTTTTTCCTTTAGGATTT |
| | | TCACACACCATATTTCTTTCACCTTTAAACTCTTACCTGGCCAACCCTATCCACCCTCTGGATCCCAATA |
| | | TTGAGATCTTATCCTCAGGGAATCCTCACTTAGACCCCTGTAACAGGTTAAATCTTCATGGTGTTCTGTT |
| | | TCCTAGGAACTTCTTTCTTTTCTACTGTTTATGACAACTGAAGTTAATAAGTGTTTATCTTTCCCACCTA |
| | | CTCAAAGTAGTTCCAAGATTAGGGCTAGTTTGTAATTCTGTGGACCACTGTAAACGAGGGCCTAGTTCAG |
| | | TGTCTGCCTCATGGGAAGCTTCCAATAAATACCTTTGCTCAACGAAAAAATGAAAACCCAGTGGCTCACG |
| | | CCTGTAATCCCAGCACTTTGGGAGGCCGAGGCAGGTGGATTGCCTGAGGTCAGGAGTTTGAGACTAGTCT |
| | | GGCCAACATGGTGAAACACTATCTCTACTAAAAATACAAAAAAATTAGCTGGGTATGGTGGCTTACGCCT |
| | | ATAATCCCAGCTACTCAGGAGGCTGAGGCAGGGGAATTGCTTGAACCAGGGAGGTGGAGGTTGCAGTGAG |
| | | CTGAGATCGCACCACTGCACTCCAGCCTGGGTGACAGAGCGAGACTCCATCTCAAAATAAAAAAGTAAAA |
| | | AAAAAAAAAAAAAA |
| 27 | T cell receptor | >gi\|54607136\|ref\|NM_016388.2\|Homo sapiens T cell receptor associated |
| | associated | transmembrane adaptor 1 (TRAT1), mRNA |
| | transmembrane | GAGGCACAGATAAAGATAAGTTTTACTGTCATGCTGCTTTTAACATAACAGAGCAACATCACCTAGGAAA |
| | adaptor 1 | AAAGTTTGTAGGAGGATTTTTAATCCATATATTTGTCTTATGGCTAGATAAAGATTTCTCTGAAAAAAAG |
| | | AAGCATGTCAGGAATCTCTGGGTGCCCCTTTTTCCTCTGGGGACTTCTAGCATTGTTGGGCTTGGCTTTG |
| | | GTTATATCACTGATCTTCAATATTrCCCACTATGTCGAAAAGCAACGACAAGATAAAATGTACAGCTACT |
| | | CCAGTGACCACACCAGGGTTGATGAGTATTATATTGAAGACACACCAATTTATGGTAACTTAGATGATAT |
| | | GATTTCAGAACCAATGGATGAAAATTGCTATGAACAAATGAAAGCCCGACCAGAGAAATCTGTAAATAAG |
| | | ATGCAGGAAGCCACCCCATCTGCACAGGCAACCAATGAAACACAGATGTGCTACGCCTCACTTGATCACA |
| | | GCGTTAAGGGGAAGCGTAGAAAGCCCAGGAAACAGAATACTCATTTCTCAGACAAGGATGGAGATGAGCA |
| | | ACTACATGCAATAGATGCCAGCGITTCTAAGACCACCTTAGTAGACAGTTTCTCCCCAGAAAGCCAGGCA |
| | | GTAGAGGAAAACATTCATGATGATCCCATCAGACTGTTTGGATTGATCCGTGCTAAGAGAGAACCTATAA |
| | | ACTAGCTGGACCATGATCTAGTTCAATGATTTGGCTCCTATTGAAGATGGCTTCTAAGAAAACAAGATGC |
| | | ACAGAGGACACAGAAGGACTTGGCAGCAGGGTGATGACCTGATCATTTGTTGATGGGATGGTGGCTTACC |
| | | TCTTATTCACAGCTTACACTTATGCATGCCAAATGTAAGGCCATGAAAATCAGTATTTCAAATAACTTAA |
| | | AAAATGCTTTACTACTAAAATGTAAAAAATTAATGTGCTCACCTCGGCAGCACATATACTAAAAATTAAT |
| | | AAGACCCAGCTTGAAAATTGAGCCTGATAACAAGATTACAAATTCACAATACCTAATACTTAGGGAAATA |
| | | TAAAAATTTAAGCATGAATGCGTTCTGGAACACGTTAGAAGAAAAATAAAAGCCAATGAGTTTTTTTTTA |
| | | ATTCTCCTTTCTCACCAATGGGCAATAGCCCATAATTGAIATAAATTTCTGATTGAAAGGTATAGGAAAC |
| | | ATTAAAATGCATTACTAAGAGAAGTAATATAATTTTCTTACAAAGTATTTTTCCCAAAGATAGCTTTACT |
| | | ATTTCAAAAATTGTCAAATTAATGCATGCTCCTTACAACAAACAAATATCAAAAAGAGTTTAGGAATTCT |
| | | ACTAGCCAGAGATAGTCACTTGGAGAAACTTTCTATATATCCTTCTAAATATTTTTCTGGGCATGCTTAT |
| | | GTATGTACATCAGTTGTTTCTTTTTATTTTGAACCAAAAATGTGGTTTCTTTTGTACACATTACTTAAAC |
| | | TTTCTTTCCAGTCAACAATATATTGTGGATTTATTTTCACTGTTATATTTAACTATATATAAATACGCAT |
| | | ATATTGTAATTTTAATGTCTGCTTAGCACCCCACTGATAACCAAATCACAGTTTATTTAAATAATTTTAA |
| | | TGACTTTTCAAAAACAATTTATTGATGCAAAAAGCAAGGTTGAGATGACAATGTTTCTTTCAATAATTAA |
| | | AAAATACTGCTTCAC |
| 28 | SLAM family | >gi\|19923571\|ref\|NN_021181.3\|Homo sapiens SLAM family member 7 (SLAMF7), |
| | member 7 | mRNA |
| | | CTTCCAGAGAGCAATATGGCTGGTTCCCCAACATGCCTCACCCTCATCTATATCCTTTGGCAGCTCACAG |
| | | GGTCAGCAGCCTCTGGACCCGTGAAAGAGCTGGTCGGTTCCGTTGGTGGGGCCGTGACTTTCCCCCTGAA |
| | | GTCCAAAGTAAAGCAAGTTGACTCTATTGTCTGGACCTTCAACACAACCCCTCTTGTCACCATACAGCCA |
| | | GAAGGGGGCACTATCATAGTGACCCAAAATCGTAATAGGGAGAGAGTAGACTTCCCAGATGGAGGCTACT |
| | | CCCTGAAGCTCAGCAAACTGAAGAAGAATGACTCAGGGATCTACTATGTGGGGATATACAGCTCATCACT |
| | | CCAGCAGCCCTCCACCCAGGAGTACGTGCTGCATGTCTACGAGCACCTGTCAAAGCCTAAAGTCACCATG |
| | | GGTCTGCAGAGCAATAAGAATGGCACCTGTGTGACCAATCTGACATGCTGCATGGAACATGGGGAAGAGG |
| | | ATGTGATTTATACCTGGAAGGCCcTGGGGCAAGCAGCCAATGAGTCCCATAATGGGTCCATCCTCCCCAT |
| | | CTCCTGGAGATGGGGAGAAAGTGATATGACCTTCATCTGCGTTGCCAGGAACCCTGTCAGCAGAAACTTC |
| | | TCAAGCCCCATCCTTGCCAGGAAGCTCTGTGAAGGTGCTGCTGATGACCCAGATTCCTCCATGGTCcTCC |
| | | TGTGTCTCcTGTTGGTGCCCCTCCTGCTCAGTCTCTTTGTACTGGGGCTATTTCTTTGGTTTCTGAAGAG |
| | | AGAGAGACAAGAAGAGTACATTGAAGAGAAGAAGAGAGTGGACATTTGTCGGGAAACTCCTAACATATGC |
| | | CCCCATTCTGGAGAGAACACAGAGTACGACACAATCCCTCACACTAATAGAACAATCCTAAAGGAAGATC |
| | | CAGCAAATACGGTTTACTCCACTGTGGAAATACCGAAAAAGATGGAAAATCCCCACTCACTGCTCACGAT |
| | | GCCAGACACACCAAGGCTATTTGCCTATGAGAATGTTATCTAGACAGCAGTGCACTCCCCTAAGTCTCTG |
| | | CTCAAAAAAAAAACAATTCTCGGCCCAAAGAAAACAATCAGAAGAATTCACTGATTTGACTAGAAACATC |
| | | AAGGAAGAATGAAGAACGTTGACTTTTTTCCAGGATAAATTATCTCTGATGCTTCTTTAGATTTAAGAGT |
| | | TCATAATTCCATCCACTGCTGAGAAATCTCCTCAAACCCAGAAGGTTTAATCACTTCATCCCAAAAATGG |
| | | GATTGTGAATGTCAGCAAACCATAAAAAAAGTGCTTAGAAGTATTCCTATAGAAATGTAAATGCAAGGTC |
| | | ACACATATTAATGACAGCCTGTTGTATTAATGATGGCTCCAGGTCAGTGTCTGGAGTTTCATTCCATCCC |
| | | AGGGCTTGGATGTAAGGATTATACCAAGAGTCTTGCTACCAGGAGGGCAAGAAGACCAAAACAGACAGAC |
| | | AAGTCCAGCAGAAGCAGATGCACCTGACAAAAATGGATGTATTAATTGGCTCTATAAACTATGTGCCCAG |
| | | CACTATGCTGAGCTTACACTAATTGGTCAGACGTGCTGTCTGCCCTCATGAAATTGGCTCCAAATGAATG |
| | | AACTACTTTCATGAGCAGTTGTAGCAGGCCTGACCACAGATTCCCAGAGGGCCAGGTGTGGATCCACACG |
| | | ACTTGAAGGTCAAAGTTCACAAAGATGAAGAATCAGGGTAGCTGACCATGTTTGGCAGATACTATAATGG |
| | | AGACACAGAAGTGTGCATGGCCCAAGGACAAGGACCTCCAGCCAGGCTTCATTTATGCACTTGTGCTGCA |
| | | AAAGAAAAGTCTAGGTTTTAAGGCTGTGCCAGAACCCATCCCAATAAAGAGACCGAGTCTGAAGTCACAT |
| | | TGTAAATCTAGTGTAGGAGACTTGGAGTCAGGCAGTGAGACTGGTGGGGCACGGGGGGCAGTGGGTACTT |
| | | GTAAACCTTTAAAGATGGTTAATTCATTCAATAGATATTTATTAAGAACCTATGCGGCCCGGCATGGTGG |
| | | CTCACACCTGTAATCCCAGCACTTTGGGAGGCCAAGGTGGGTGGGTCATCTGAGGTCAGGAGTTCAAGAC |
| | | CAGCCTGGCCAACATGGTGAAACCCCATCTCTACTAAAGATACAAAAATTTGCTGAGCGTGGTGGTGTGC |
| | | ACCTGTAATCCCAGCTACTCGAGAGGCCAAGGCATGAGAATCGCTTGAACCTGGGAGGTGGAGGTTGCAG |
| | | TGAGCTGAGATGGCACCACTGCACTCCGGCCTAGGCAACGAGAGCAAAACTCCAATACAAACAAACAAAC |
| | | AAACACCTGTGCTAGGTCAGTCTGGCACGTAAGATGAACATCCCTACCAACACAGAGCTCACCATCTCTT |
| | | ATACTTAAGTGAAAAACATGGGGAAGGGGAAAGGGGAATGGCTGCTTTTGATATGTTCCCTGACACATAT |
| | | CTTGAATGGAGACCTCCCTACCAAGTGATGAAAGTGTTGAAAAACTTAATAACAAATGCTTGTTGGGCAA |
| | | GAATGGGATTGAGGATTATCTTCTCTCAGAAAGGCATTGTGAAGGAATTGAGCCAGATCTCTCTCCCTAC |
| | | TGCAAAACCCTATTGTAGTAAAAAAGTCTTCTTTACTATCTTAATAAAACAGATATTGTGAGATTCAAAA |
| | | AAAAAAAAAAAA |
| 29 | chromosome 4 open | >gi\|50428928\|ref\|NM_152997.2\|Homo sapiens chromosome 4 open reading frame |
| | reading frame 7 | 7 (C4orf7), mRNA |
| | | TAAAACAGCTACAATATTCCAGGGCCAGTCACTTGCCATTTCTCATAACAGCGTCAGAGAGAAAGAACTG |
| | | ACTGAAACGTTTGAGATGAAGAAAGTTCTCCTCCTGATCACAGCCATCTTGGCAGTGGCTGTTGGTTTCC |
| | | CAGTCTCTCAAGACCAGGAACGAGAAAAAAGAAGTATCAGTGACAGCGATGAATTAGCTTCAGGGTTTTT |
| | | TGTGTTCCCTTACCCATATCCATTTCGCCCACTTCCACCAATTCCATTTCCAAGATTTCCATGGTTTAGA |
| | | CGTAATTTTCCTATTCCAATACCTGAATCTGCCCCTACAACTCCCCTTCCTAGCGAAAAGTAAACAAGAA |
| | | GGAAAAGTCACGATAAACCTGGTCACCTGAAATTGAAATTGAGCCACTTCCTTGAAGAATCAAAATTCCT |
| | | GTTAATAAAAGAAAAACAAATGTAATTGAAATAGCACACAGCATTCTCTAGTCAATATCTTTAGTGATCT |
| | | TCTTTAATAAACTTGAAAGCAAAGATTTTGGTTTCTTAATTTCCACAAA |
| 30 | Major histo- | >gi\|52426772\|ref\|NM_002122.3\|Homo sapiens major histocompatibility |
| | compatibility | complex, class II, DQ alpha 1 (HLA-DQA1), mRNA |
| | complex, class | ACAATTACTCTACAGCTCAGAACACCAACTGCTGAGGCTGCCTTGGGAAGAGGATGATCCTAAACAAAGC |
| | II, DQ alpha 1 | TCTGCTGCTGGGGGCCCTCGCTCTGACCACCGTGATGAGCCCCTGTGGAGGTGAAGACATTGTGGCTGAC |
| | | CACGTTGCCTCTTGTGGTGTAAACTTGTACCACTTTTACGGTCCCTCTGGCCAGTACACCCATGAATTTG |
| | | ATGGAGATGAGCAGTTCTACGTGGACCTGGAGAGGAAGGAGACTGCCTGOCGGTGGCCTGAGTTCAGCAA |
| | | ATTTGGAGGTTTTGACCCGCAGGGTGCACTGAGAAACATGGCTGTGGCAAAACACAACTTGAACATCATG |
| | | ATTAAACGCTACAACTCTACCGCTGCTACCAATGAGGTTCCTGAGGTCACAGTGTTTTCCAAGTCTCCCG |
| | | TGACACTGGGTCAGCCCAACACCCTCATTTGTCTTGTGGACAACATCTTTCcTCCTGTGGTCAACATCAC |
| | | ATGGCTGAGCAATGGGCAGTCAGTCACAGAAGGTGTTTCTGAGACCAGCTTCCTCTCCAAGAGTGATCAT |
| | | TCCTTCTTCAAGATCAGTTACCTCACCTTCCTCCCTTCTGCTGATGAGATTTATGACTGCAAGGTGGAGC |
| | | ACTGGGGCCTGGACCAGCCTCTTGTGAAACACTGGGAGCCTGAGATTCCAGCCCCTATGTCAGAGCTCAC |
| | | AGAGACTGTGGTCTGTGCCCTGGGGTTGTCTGTGGGCCTCATGGGCATTGTGGTGGGCACTGTCTTCATC |
| | | ATCCAAGGCCTGCGTTCAGTTGGTGCTTCCAGACACCAAGGGCCATTGTGAATCCCATCCTGGAAGGGAA |
| | | GGTGCATCGCCATCTACAGGAGCAGAAGAATGGACTTGCTAAATGACCTAGCACTATTCTCTGGCCCGAT |
| | | TTATCATATCCCTTTTCTCCTCCAAATATTTCTCCTCTCACCTTTTCTCTGGGACTTAAGCTGCTATATC |
| | | CCCTCAGAGCTCACAAATGCCTTTACATTCTTTCCCTGACCTCCTGATTTTTTTTTTCTTTTCTCAAATG |
| | | TTACCTACAAAGACATGCCTGGGGTAAGCCACCCGGCTACCTAATTCCTCAGTAACCTCCATCTAAAATC |
| | | TCCAAGGAAGCAATAAATTCCTTTTATGAGATCTATGTCAAATTTTTCCATCTTTCATCCAGGGCTGACT |
| | | GAAACTATGGCTAATAATTGGGGTACTCTTATGTTTCAATCCAATTTAACCTCATTTCCCAGATCATTTT |
| | | TCATGTCCAGTAACACAGAAGCCACCAAGTACAGTATAGCCTGATAATATGTTGATTTCTTAGCTGACAT |
| | | TAATATTTCTTGCTTCCTTGTGTTCCCACCCTTGGCACTGCCACCCACCCCTCAATTCAGGCAACAATGA |
| | | AATTAATGGATACCGTCTGCCCTTGGCCCAGAATTGTTATAGCAAAAATTTTAGAACCAAAAAATAAGTC |
| | | TGTACTAATTTCAATGTGGCTTTTAAAAGTATGACAGAGAAATAAGTTAGGATAAAGGAAATTTGAATCT |
| | | CA |
| 31 | Transcribed locus | >gi\|3807951\|gb\|AI225238.1\|AI225238 qx12c04.x1 NCI_CGAP_Lym12 Homo sapiens |
| | | cDNA clone IMAGE: 2001126 3′, mRNA sequence |
| | | AGCGGCCGCCCTTTTTTTTTTTTTTTGAATGTTTTTATATTTTTATGTTTTTCTCTCTCACCACATTAGA |
| | | GATTCACATGTCAAAAAAAAAGTTCAAAGATTCCACTTAAATGGTAGTCAAAATTAAAAGCATGTTGTTT |
| | | AATTTGCCAACTATTCCATAAATCCTGTGCTTGAAGCACTACCTTTTATCAACCACTTGAAAGGAAGGAA |
| | | TATGAAGTGTGACTCTTGAGGATTGCAGTTCCATTTCTCCACAGACATAGCAGGGTGTGTAGTTCCAAAT |
| | | GGGTTTCTTCCAAAGTCATCTCCTCAGAGTAGGACTGTCTGAGTAGTCATTCTGATAATGTTGCCATTAA |
| | | CTACAAGCACATCTGAGAACCCTCGTAAGGAAGTGCCATTAGATTCAGA |
| 32 | Amphiphysin (Stiff- | >gi\|51466133\|ref\|XM_499518.1\|PREDICTED: Homo sapiens LOC442534 |
| | Man syndrome with | (LOC442534), mRNA |
| | breast cancer | GGCCCAGCCCTAGGAGGGGGCAGTCCCACAGCAGGCTCACACCCTGCCCTTCAGAGTTGCAGCAGCTAAT |
| | 128 kDa autoantigen) | GCTGATGCCAGGACCTTGTCCTGCCAGGAAGCCTCTGATTAGGGCTGTGAACCAACTTCCTTTTCACATC |
| | | TGCAAGGACTCTGCTCTAAGCTCAAATTTTGCAGTTTCTGCTGTCATCACAGCTGGGAATGAGAAAGGGA |
| | | AGGGGCTCCAGTTGGTCTCCCTCTGCCTCTGGAGCACACGTGGAGGACATGAGGCTTCCGCAGGAACAGC |
| | | TCTGAAAGCTGAGTTCTCGGGTCCCTGTGAAGGAGGGAAGCTTGCCTGCCTTGGAGCGTGAAAAAGTCTT |
| | | GCTGGACAGAAGTCTCACGGTTTAAAACTGGGAAGGGAGAGGATTGAGATCACACTAGAATGCACAGAAA |
| | | ATTCTT |
| 33 | dendritic cell- | >gi\|40548404\|ref\|NM_172004.2\|Homo sapiens dendritic cell-associated |
| | associated lectin-1 | lectin-1 (DCAL1), mRNA |
| | | ATGGTTAGTAATTTCTTCCATGTCATACAAGTATTCGAGAAATCTGCTACCTTGATTAGTAAGACTGAAC |
| | | ACATTGGTTTTGTCATTTATTCATGGAGGAAGTCCACCACCCACTTGGGGAGCAGAAGGAAATTTGCCAT |
| | | CTCAATTTACTTATCAGAAGTTTCTTTGCAGAAATATGATTGTCCCTTCAGTGGGACATCATTTGTGGTC |
| | | TTCTCTCTCTTTTTGATCTGTGCAATGGCTGGAGATGTAGTCTACGCTGACATCAAAACTGTTCGGACTT |
| | | CCCCGTTAGAACTCGCGTTTCCACTTCAGAGATCTGTTTCTTTCAACTTTTCTACTGTCCATAAATCATG |
| | | TCCTGCCAAAGACTGGAAGGTGCATAAGGGAAAATGTTACTGGATTGCTGAAACTAAGAAATCTTGGAAC |
| | | AAAAGTCAAAATGACTGTGCCATAAACAATTCATATCTCATGGTGATTCAAGACATTACTGCTATGGTGA |
| | | GATTTAACATTTAGAGGTGACAGCATCCCCCACACTGGCAGTGAATTTTTTGTGCTACAAACTTGGCAAA |
| | | AGTCTGTGAAAAGAAGTTTCAACTTCATGTGTTATTAACTATACAAATATTAGTTGAATGAATTGTTGAA |
| | | TTACAAAAAAAAAAAAAAA |

TABLE 1B
Probes for identifying genes of Table 1 (and their fold upregulation in Mage cancer patients)

| | | | | |
|---|---|---|---|---|
| Probe | | | | Fold |
| set ID | Gene name | Probeset sequence | Full gene sequence | change |

| | | | | |
|---|---|---|---|---|
| 1558972_ | chromosome | ttctaagacccacatttggttatt | >gi\|58218985\|ref\|NM_001010923.1\|Homo sapiens | 44.93 |
| s_at | 6 open | gaaggccacagcgaatcttaacct | chromosome 6 open reading frame 190 (C6orf190), | |
| | reading | aacagccttgac | mRNA | |
| | frame 190 | aaactgcaccataggtgtttttag | ATACTTACAATTACGAGATTTATATTTGCATTAGTCTCTTTGGCTGG | |
| | | actcatataatttgttatttttca | TGGGTAGGGGTGAGAGGCTCTTC | |
| | | aacaatagtgaa | CTGGATCCCTTATTTTCTACAGGAGAGGAGGAAAACACCTGGGATGC | |
| | | taattaatattnttgtttggaatt | TCCAGTGCTCTTACGCAGATAAT | |
| | | tgagnacaattaaatttgtacttt | GATCATTAACATCAGCCTCTCTGATCAAAGAGCTACTCCACCCCACT | |
| | | tagtaactacca | CTGGCTGTAGTGTGACATTCCTG | |
| | | ttctttgattagaaaattaagaga | CCTGCCTGAGGAAGAAATGGTGAGCAGGGGCTGCAATTGCAGACAAG | |
| | | atgcatatcttactttggttgtaa | TGTCACCCAGAAGCCACAAGTTT | |
| | | attatcaagggc | CTGTGAGCACCAGGTCTACAAACTACCCAAGGCATAGCAATGGCATT | |
| | | tttctaatagaaatcatatataac | ATCACTGGAAGAATTCGTCCACT | |
| | | atttctaaatataagtcctttcac | CCCTTGACCTCAGGACCCTACCCAGGGTTCTAGAAATCCAGGCAGGC | |
| | | atactgtgtttc | ATCTATCTTGAAGGCTCTATTTA | |
| | | cagttgtcttgatattgaaaagtg | TGAAATGTTTGGAAATGAATGCTGTTTTTCAACAGGAGAAGTGATTA | |
| | | taataaacttcatgctcacctat | AAATTACTGGTCTCAAAGTTAAG | |
| | | [Seq ID No 49] | AAGATCATAGCTGAAATTTGTGAGCAGATTGAAGGTTGTGAGTCTCT | |
| | | | ACAGCCATTTGAACTGCCTATGA | |
| | | | ATTTTCCAGGTCTTTTTAAGATTGTGGCTGATAAAACTCCATACCTT | |
| | | | ACTATGGAAGAAATCACAAGGAC | |
| | | | CATTCATATTGGACCAAGTAGACTAGGGCATCCTTGCTTCTATCATC | |
| | | | AGAAGGATATAAAACTAGAGAAC | |
| | | | CTCATCATAAAGCAGGGTGAGCAAATCATGCTCAACTCAGTTGAAGA | |
| | | | GATTGATGGAGAAATAATGGTGA | |
| | | | GCTGTGCAGTAGCAAGGAATCATCAAACTCACTCATTTAATTTGCCT | |
| | | | TTGTCACAAGAAGGAGAATTCTA | |
| | | | CGAGTGTGAAGATGAACGTATTTACACTCTAAAGGAGATTGTTGAAT | |
| | | | GGAAGATTCCTAAGAACAGAACA | |
| | | | AGAACTGTAAACCTTACAGATTTTTCAAATAAGTGGGACTCAACGAA | |
| | | | TCCATTTCCTAAAGACTTTTATG | |
| | | | GTACCCTGATTCTCAAGCCTGTTTATGAAATTCAAGGTGTGATGAAA | |
| | | | TTTCGAAAAGATATAATCCGCAT | |
| | | | CCTCCCCAGTCTAGATGTCGAAGTCAAAGACATCACTGATTCTTACG | |
| | | | ATGCTAACTGGTTTCTTCAGCTG | |
| | | | TTATCAACAGAAGATCTTTTTGAAATGACTAGTAAAGAGTTCCCCAT | |
| | | | AGTGACTGAAGTCATAGAAGCAC | |
| | | | CTGAAGGAAACCACCTGCCCCAAAGCATTTTACAGCCTGGGAAAACC | |
| | | | ATTGTGATCCACAAAAAGTACCA | |
| | | | GGCATCAAGAATCTTAGCTTCAGAAATTAGAAGCAATTTTCCTAAAA | |
| | | | GACACTTCTTGATCCCCACTAGC | |
| | | | TATAAAGGCAAGTTCAAGCGGCGACCGAGGGAGTTCCCAACGGCCTA | |
| | | | TGACCTAGAGATCGCTAAGAGTG | |
| | | | AAAAGGAGCCTCTTCACGTGGTGGCCACCAAAGCGTTTCATTCCCCT | |
| | | | CATGACAAGCTGTCATCCGTATC | |
| | | | TGTTGGGGACCAGTTTCTGGTGCATCAGTCAGAGACGACTGAAGTCC | |
| | | | TCTGTGAGGGAATAAAAAAAGTG | |
| | | | GTGAATGTTCTGGCCTGTGAAAAAATCCTCAAAAAGTCCTATGAGGC | |
| | | | TGCGCTGCTCCCTTTGTACATGG | |
| | | | AAGGAGGTTTTGTAGAGGTGATTCATGATAAGAAACAGTACCCGATT | |
| | | | TCTGAGCTCTGTAAACAGTTCCG | |
| | | | TTTGCCCTTCAATGTGAAGGTGTCTGTCAGGGATCTTTCCATTGAAG | |
| | | | AGGACGTGTTGGCTGCCACACCA | |
| | | | GGACTGCAGTTGGAGGAGGACATTACAGACTCTTACCTACTCATAAG | |
| | | | TGACTTTGCCAACCCCACGGAGT | |
| | | | GCTGGGAAATTCCTGTGGGCCGCTTGAATATGACTGTTCAGTTAGTT | |
| | | | AGTAATTTCTCTAGGGATGCAGA | |
| | | | ACCATTTCTAGTCAGGACTCTGGTAGAAGAGATCACTGAAGAGCAAT | |
| | | | ATTACATGATGCGGAGATATGAA | |
| | | | AGCTCAGCCTCACATCCCCCACCTCGCCCTCCGAAACACCCCTCAGT | |
| | | | AGAGGAAACAAAGTTAACCCTGC | |
| | | | TAACCTTAGCAGAAGAAAGGACGGTAGACCTGCCCAAGTCTCCCAAG | |
| | | | CGTCATCACGTAGACATAACCAA | |
| | | | GAAACTTCACCCAAATCAAGCTGGCCTGGATTCAAAAGTACTGATTG | |
| | | | GTAGTCAGAATGATTTGGTGGAT | |
| | | | GAAGAGAAAGAAAGGAGCAACCGTGGGGCCACAGCAATAGCAGAAAC | |
| | | | ATTCAAAAATGAAAAACATCAAA | |
| | | | AATAACAAGATGTGACAGAAGCCACTTAGGCAGCAAACATAAATGTT | |
| | | | GCAGTGAAAAAAGAAGCTAGCCT | |
| | | | TCTAGCTGAAAAACGAGTATTCCCCAATGGACTCCAGAAGAAACTTG | |
| | | | ATTCATCGCTGCAAAGGAAAGAA | |
| | | | CAACCTTAAAACTTTTAACAGATAAAACTTACAGAAACCTATGATAT | |
| | | | AGAATTCATATAGTCTATTCTGT | |
| | | | TGTGTCTAAATCTGTAGGCATTGTGTTGTTGTTCTTTAGGACGTATT | |
| | | | TATTTAACTTGCACATTTTTTCA | |
| | | | GATTCTTATTTCTACTACCAACAACTAAGTAATTGGGAAATAATTCT | |
| | | | GTATTTCAGTTTCTGAGTAAAAC | |
| | | | CAGTCTGAAATAGGATAAAAGCCACCAAATATTTTCTTTTTTTTCCA | |
| | | | GAATTTGTTTTGCCATTTTTTAG | |
| | | | TGCTATCATCATTCCTAACAAGACTAACTTACAGAAAAATAATTATA | |
| | | | TCTGACTGATTTAAAATGTTCAG | |
| | | | GTTTCTTATCCAAATCCCTTGGAACTATGGAAAGGAGTTTGATTTCA | |
| | | | CATTCACAGTGTATTTACAAAAT | |
| | | | ACGCTGTGTCATAAATATGTTTGAATTCCAACAGCCAAAGCCATTGA | |
| | | | GAGTCATAGGAGTTTTCCATAAC | |
| | | | CTTCTCTTCTATGACCCAACAACAAGCTCATGACTGAAATTTCACCA | |
| | | | GATTTCTGAGACGATGTCTTAAT | |
| | | | ATTCTATGTGCTATGTACCAGATAATTCTTTAGATGAATGTTTCTTA | |
| | | | GGATTGTAGGAAAATTATCTAGT | |
| | | | TAATCATAATATTTGATGGAAAGAAAAAGACAATAAAATTGTAATAT | |
| | | | AATAAATTTGGCTGACAAGAAAC | |
| | | | CAAAGTGATTCTTAATTAGTATACATCAGAATGATGCTCTTATAGTT | |
| | | | GTACCATCTATAAAAATTACTTT | |
| | | | AAGGGCTCTCACATTTTAATAATTTATCTTATTATGTATTAAGTATA | |
| | | | CAGGAACAATATTATTTTTCCTT | |
| | | | TAACAAAATGAAGAGACAGGCTATCTGGTTAATGTTACATAGGAATT | |
| | | | TAATAGTAATGCTTGAACTTCAT | |
| | | | CCATAGATCATACTCTGTACAAAATTTGTTAGCTAACATCCTATCTC | |
| | | | ATAATTATTTTATGTTTTGTGGA | |
| | | | GAAATTTGTTGATTTTGTACCAAAGTGTTTCTGAAGACAATAAATTG | |
| | | | TGAGTCAACTTTAGAACAAAAAA | |
| | | | AATTAGAGTTTTTTCAATGTTTATATTCTGATTAAGCTTACTTTACC | |
| | | | TTACATTTTTTCTAAGTAACAAT | |
| | | | GAATCCTGATTTCTAGTGTCCTAAAAATTGCTTAGTGATTTGATTGT | |
| | | | GGTAATATCATTTCTTATCTACA | |
| | | | ATGTCTAAAGTTTTATGGGACAGTTTTTCTTTTATTTATTTTGCCTG | |
| | | | TTTGTGCAGATAAGAACAGAAAC | |
| | | | TTTTCTAAGACCCACATTTGGTTATTGAAGGCCACAGCGAATCTTAA | |
| | | | CCTAACAGCCTTGACAAACTGCA | |
| | | | CCATAGGTGTTTTTAGACTCATATAATTTGTTATTTTTCAAACAATA | |
| | | | GTGAATAATTAATATTTTTGTTT | |
| | | | GGAATTTGAGAACAATTAAATTTGTACTTTTAGTAACTACCATTCTT | |
| | | | TGATTAGAAAATTAAGAGAATGC | |
| | | | ATATCTTACTTTGGTTGTAAATTATCAAGGGCTTTCTAATAGAAATC | |
| | | | ATATATAACATTTCTAAATATAA | |
| | | | GTCCTTTCACATACTGTGTTTCCAGTTGTCTTGATATTGAAAAGTGT | |
| | | | AATAAACTTCATGCTCACCTATT | |
| | | | GGAGATTTGGGAAGGTTGAAAATAAACTTCCTAATTTTTAAAAAAAA | |
| | | | AA | |
| 202957_ | hematopoietic | ttgctggcttctcttatactaacc | >gi\|37059786\|ref\|NM_005335.3\|Homo sapiens | 19.09 |
| _at | cell- | cagagtttgtcattaatgtgtagg | hematopoietic cell-specific Lyn substrate 1 | |
| | specific | tgaatgcaaact | (HCLS1), mRNA | |
| | Lyn | ccatcgttgagcctggggtgtaag | GTGGACGCGAGGAGCCGGGCGCTTAGAACAGAGGCTTGCACAGGTGG | |
| | substrate 1 | acttcaagccaagcgtatgtatca | AGATGTGGAAGTCTGTAGTGGGC | |
| | | attctagtcttc | CATGATGTGTCTGTTTCCGTGGAGACCCAGGGTGATGATTGGGACAC | |
| | | caggattcacggtgcacatgctgg | AGATCCTGACTTTGTGAATGACA | |
| | | cattcaacatgtggaaagcttgtc | TCTCTGAAAAGGAGCAACGATGGGGAGCCAAGACCATCGAGGGGTCT | |
| | | ttagaggccttt | GGACGCACAGAACACATCAACAT | |
| | | cttgtatgtgtagcttgctagttt | CCACCAGCTGAGGAACAAAGTATCAGAGGAGCATGATGTTCTCAGGA | |
| | | gttttctacatttgaaaatgttta | AGAAAGAGATGGAGTCAGGGCCC | |
| | | gtttagaataag | AAAGCATCCCATGGCTATGGAGGTCGGTTTGGAGTAGAAAGAGACCG | |
| | | cgcattatccaattatagaggtac | AATGGACAAGAGTGCAGTGGGCC | |
| | | aattttccaaacttccagaaactc | ATGAGTATGTTGCCGAGGTGGAGAAGCACTCTTCTCAGACGGATGCT | |
| | | atcaaatgaaca | GCCAAAGGCTTTGGGGGCAAGTA | |
| | | gacaatgtcaaaactactgtgtct | CGGAGTTGAGAGGGACAGGGCAGACAAGTCAGCAGTCGGCTTTGATT | |
| | | gataccaaaatgcttcagtatttg | ATAAAGGAGAAGTGGAGAAGCAT | |
| | | taatttttcaag | ACATCTCAGAAAGATTACTCTCGTGGCTTTGGTGGCCGGTACGGGGT | |
| | | tcagaagctgatgttcctggtaaa | GGAGAAGGATAAATGGGACAAAG | |
| | | agtt | CAGCTCTGGGATATGACTACAAGGGAGAGACGGAGAAACACGAGTCC | |
| | | [Seq ID No 50] | CAGAGAGATTATGCCAAGGGCTT | |
| | | | TGGTGGCCAGTATGGAATCCAGAAGGACCGAGTGGATAAGAGCGCTG | |
| | | | TCGGCTTCAATGAAATGGAGGCC | |
| | | | CCGACCACAGCTTATAAGAAGACGACGCCCATAGAAGCCGCTTCTAG | |
| | | | TGGTGCCCGTGGGCTGAAGGCGA | |
| | | | AATTTGAGTCCATGGCTGAGGAGAAGAGGAAGCGAGAGGAAGAGGAG | |
| | | | AAGGCACAGCAGGTGGCCAGGAG | |
| | | | GCAACAGGAGCGAAAGGCTGTGACAAAGAGGAGCCCTGAGGCTCCAC | |
| | | | AGCCAGTGATAGCTATGGAAGAG | |
| | | | CCAGCAGTACCGGCCCCACTGCCCAAGAAAATCTCCTCAGAGGCCTG | |
| | | | GCCTCCAGTTGGGACTCCTCCAT | |
| | | | CATCAGAGTCTGAGCCTGTGAGAACCAGCAGGGAACACCCAGTGCCC | |
| | | | TTGCTGCCCATTAGGCAGACTCT | |
| | | | CCCGGAGGACAATGAGGAGCCCCCAGCTCTGCCCCCTAGGACTCTGG | |
| | | | AAGGCCTCCAGGTGGAGGAAGAG | |
| | | | CCAGTGTACGAAGCAGAGCCTGAGCCTGAGCCCGAGCCTGAGCCCGA | |
| | | | GCCTGAGAATGACTATGAGGACG | |
| | | | TTGAGGAGATGGACAGGCATGAGCAGGAGGATGAACCAGAGGGGGAC | |
| | | | TATGAGGAGGTGCTCGAGCCTGA | |
| | | | AGATTCTTCTTTTTCTTCTGCTCTGGCTGGATCATCAGGCTGCCCGG | |
| | | | CTGGGGCTGGGGCTGGGGCTGTG | |
| | | | GCTCTGGGGATCTCAGCTGTGGCTCTATATGATTACCAAGGAGAGGG | |
| | | | AAGTGATGAGCTTTCCTTTGATC | |
| | | | CGGACGACGTAATCACTGACATTGAGATGGTGGACGAGGGCTGGTGG | |
| | | | CGGGGACGTTGCCATGGCCACTT | |
| | | | TGGACTCTTCCCTGCAAATTATGTCAAGCTTCTGGAGTGACTAGAGC | |
| | | | TCACTGTCTACTGCAACTGTGAT | |
| | | | TTCCCATGTCCAAAGTGGCTCTGCCTCCACCCCCTCCCTATTCCTGA | |
| | | | TGCAAATGTCTAACCAGATGAGT | |
| | | | TTCTGGACAGACTTCCCTCTCCTGCTTCATTAAGGGCTTGGGGCAGA | |
| | | | GACAGCATGGGGAAGGAGGTCCC | |
| | | | CTTCCCCAAGAGTCCTCTCTATCCTGGATGAGCTCATGAACATTTCT | |
| | | | CTTGTGTTCCTGACTCCTTCCCA | |
| | | | ATGAACACCTCTCTGCCACCCCAAGCTCTGCTCTCCTCCTCTGTGAG | |
| | | | CTCTGGGCTTCCCAGTTTGTTTA | |
| | | | CCCGGGAAAGTACGTCTAGATTGTGTGGTTTGCCTCATTGTGCTATT | |
| | | | TGCCCACTTTCCTTCCCTGAAGA | |
| | | | AATATCTGTGAACCTTCTTTCTGTTCAGTCCTAAAATTCGAAATAAA | |
| | | | GTGAGACTATGGTTCACCTGTAA | |
| | | | AAAAAAAAAAAAAAAAAAAAAAAAAAAAAAAAAAAAAAAAAAAAAAA | |
| | | | AAAAAAAAAAAAAAAAAAAAAA | |
| 204116_ | interleukin | ttctggctggaacggacgatgccc | >gi\|4557881\|ref\|NM_00206.1\|Homo sapiens | 61.98 |
| at | 2 receptor, | cgaattcccaccctgaagaaccta | interleukin 2 receptor, gamma (severe combined | |
| | gamma | gaggatcttgtt | immunodeficiency) (IL2RG), mRNA | |
| | (severe | actgaataccacgggaacttttcg | GAAGAGCAAGCGCCATGTTGAAGCCATCATTACCATTCACATCCCTC | |
| | combined | gcctggagtggtgtgtctaaggga | TTATTCCTGCAGCTGCCCCTGCT | |
| | immuno- | ctggctgagagtctgcagccagac | GGGAGTGGGGCTGAACACGACAATTCTGACGCCCAATGGGAATGAAG | |
| | deficiency) | tacagtgaacgactctgcctcgtc | ACACCACAGCTGATTTCTTCCTG | |
| | | agtgagattcccccaaaaggaggg | ACCACTATGCCCACTGACTCCCTCAGTGTTTCCACTCTGCCCCTCCC | |
| | | gcccttggggaggggcctggggcc | AGAGGTTCAGTGTTTTGTGTTCA | |
| | | tccccatgcaaccagcatagcccc | ATGTCGAGTACATGAATTGCACTTGGAACAGCAGCTCTGAGCCCCAG | |
| | | tactgggcccccccatgttacacc | CCTACCAACCTCACTCTGCATTA | |
| | | ctaaagcctgaaacctgaacccca | TTGGTACAAGAACTCGGATAATGATAAAGTCCAGAAGTGCAGCCACT | |
| | | atcctctgacagaagaaccccagg | ATCTATTCTCTGAAGAAATCACT | |
| | | gtcctgtagccctaagtggtacta | TCTGGCTGTCAGTTGCAAAAAAAGGAGATCCACCTCTACCAAACATT | |
| | | actttccttcattcaacccacctg | TGTTGTTCAGCTCCAGGACCCAC | |
| | | cgtctcatactcacctcaccccac | GGGAACCCAGGAGACAGGCCACACAGATGCTAAAACTGCAGAATCTG | |
| | | tgtggctgatttggaattttgtgc | GTGATCCCCTGGGCTCCAGAGAA | |
| | | ccccatgtaagcacc | CCTAACACTTCACAAACTGAGTGAATCCCAGCTAGAACTGAACTGGA | |
| | | [Seq ID No 51] | ACAACAGATTCTTGAACCACTGT | |
| | | | TTGGAGCACTTGGTGCAGTACCGGACTGACTGGGACCACAGCTGGAC | |
| | | | TGAACAATCAGTGGATTATAGAC | |
| | | | ATAAGTTCTCCTTGCCTAGTGTGGATGGGCAGAAACGCTACACGTTT | |
| | | | CGTGTTCGGAGCCGCTTTAACCC | |
| | | | ACTCTGTGGAAGTGCTCAGCATTGGAGTGAATGGAGCCACCCAATCC | |
| | | | ACTGGGGGAGCAATACTTCAAAA | |
| | | | GAGAATCCTTTCCTGTTTGCATTGGAAGCCGTGGTTATCTCTGTTGG | |
| | | | CTCCATGGGATTGATTATCAGCC | |
| | | | TTCTCTGTGTGTATTTCTGGCTGGAACGGACGATGCCCCGAATTCCC | |
| | | | ACCCTGAAGAACCTAGAGGATCT | |
| | | | TGTTACTGAATACCACGGGAACTTTTCGGCCTGGAGTGGTGTGTCTA | |
| | | | AGGGACTGGCTGAGAGTCTGCAG | |
| | | | CCAGACTACAGTGAACGACTCTGCCTCGTCAGTGAGATTCCCCCAAA | |
| | | | AGGAGGGGCCCTTGGGGAGGGGC | |
| | | | CTGGGGCCTCCCCATGCAACCAGCATAGCCCCTACTGGGCCCCCCCA | |
| | | | TGTTACACCCTAAAGCCTGAAAC | |
| | | | CTGAACCCCAATCCTCTGACAGAAGAACCCCAGGGTCCTGTAGCCCT | |
| | | | AAGTGGTACTAACTTTCCTTCAT | |
| | | | TCAACCCACCTGCGTCTCATACTCACCTCACCCCACTGTGGCTGATT | |
| | | | TGGAATTTTGTGCCCCCATGTAA | |
| | | | GCACCCCTTCATTTGGCATTCCCCACTTGAGAATTACCCTTTTGCCC | |
| | | | CGAACATGTTTTTCTTCTCCCTC | |
| | | | AGTCTGGCCCTTCCTTTTCGCAGGATTCTTCCTCCCTCCCTCTTTCC | |
| | | | CTCCCTTCCTCTTTCCATCTACC | |
| | | | CTCCGATTGTTCCTGAACCGATGAGAAATAAAGTTTCTGTTGATAAT | |
| | | | CATC | |
| 204661_ | CD52 | acagccacgaagatcctaccaaaa | >gi\|68342029\|ref\|NM_001803.2\|Homo sapiens CD52 | 30.71 |
| at | antigen | tgaagcgcttcctcttcctcctac | molecule (CD52), mRNA | |
| | (CAMPATH-1 | tcaccatcagcc | CTCCTGGTTCAAAAGCAGCTAAACCAAAAGAAGCCTCCAGACAGCCC | |
| | antigen)/// | tcctggttatggtacagatacaaa | TGAGATCACCTAAAAAGCTGCAT | |
| | CD52 | ctggactctcaggacaaaacgaca | CCAAGACAGCCACGAAGATCCTACCAAAATGAAGCGCTTCCTCTTCC | |
| | antigen | ccagccaaaccagcagcccctcag | TCCTACTCACCATCAGCCTCCTG | |
| | (CAMPATH-1 | catccagcagcatgagcggaggca | GTTATGGTACAGATACAAACTGGACTCTCAGGACAAAACGACACCAG | |
| | antigen) | ttttccttttcttcgtggccaatg | CCAAACCAGCAGCCCCTCAGCAT | |
| | | ccataatccacctcttctgcttca | CCAGCAACATAAGCGGAGGCATTTTCCTTTTCTTCGTGGCCAATGCC | |
| | | gttgaggtgacacgtctcagcctt | ATAATCCACCTCTTCTGCTTCAG | |
| | | agccctgtgccc | TTGAGGTGACACGTCTCAGCCTTAGCCCTGTGCCCCCTGAAACAGCT | |
| | | cctgaaacagctgccaccatcact | GCCACCATCACTCGCAAGAGAAT | |
| | | cgcaagagaatcccctccatcttt | CCCCTCCATCTTTGGGAGGGGTTGATGCCAGACATCACCAGGTTGTA | |
| | | gggaggggttgatgccagacatca | GAAGTTGACAGGCAGTGCCATGG | |
| | | ccaggttgtagaagttgacaggca | GGGCAACAGCCAAAATAGGGGGGTAATGATGTAGGGGCCAAGCAGTG | |
| | | gtgccatgggggcaacagccaaaa | CCCAGCTGGGGGTCAATAAAGTT | |
| | | taggggggtaatgatgtaggggcc | ACCCTTGTACTTGCAAAAAAAAAAAAAAAAAAA | |
| | | aagc | | |
| | | [Seq ID No 52] | | |
| 205831_ | CD2 | agacctcgagttcagccaaaacct | >gi\|31542293\|ref\|NM_001767.2\|Homo sapiens CD2 | 22.66 |
| at | antigen | ccccatggggcagcagaaaactca | molecule (CD2), mRNA | |
| | (p50), | ttgtccccttcctctaattaaaaa | ACCAACCCCTAAGATGAGCTTTCCATGTAAATTTGTAGCCAGCTTCC | |
| | sheep red | agatagaaactgtctttttcaata | TTCTGATTTTCAATGTTTCTTCC | |
| | blood cell | aaaagcactgtggatttctgccct | AAAGGTGCAGTCTCCAAAGAGATTACGAATGCCTTGGAAACCTGGGG | |
| | receptor/// | cctgatgtgcatatccgtacttcc | TGCCTTGGGTCAGGACATCAACT | |
| | CD2 | atgaggtgttttctgtgtgcagaa | TGGACATTCCTAGTTTTCAAATGAGTGATGATATTGACGATATAAAA | |
| | antigen | cattgtcacctc | TGGGAAAAAACTTCAGACAAGAA | |
| | (p50), | ctgaggctgtgggccacagccacc | AAAGATTGCACAATTCAGAAAAGAGAAAGAGACTTTCAAGGAAAAAG | |
| | sheep red | tctgcatcttcgaactcagccatg | ATACATATAAGCTATTTAAAAAT | |
| | blood cell | tggtcaacatct | GGAACTCTGAAAATTAAGCATCTGAAGACCGATGATCAGGATATCTA | |
| | receptor | ggagtttttggtctcctcagagag | CAAGGTATCAATATATGATACAA | |
| | | ctccatcacaccagtaaggagaag | AAGGAAAAAATGTGTTGGAAAAAATATTTGATTTGAAGATTCAAGAG | |
| | | caatataagtgt | AGGGTCTCAAAACCAAAGATCTC | |
| | | gattgcaagaatggtagaggaccg | CTGGACTTGTATCAACACAACCCTGACCTGTGAGGTAATGAATGGAA | |
| | | agcacagaaatcttagagatttct | CTGACCCCGAATTAAACCTGTAT | |
| | | tgtcccctctca | CAAGATGGGAAACATCTAAAACTTTCTCAGAGGGTCATCACACACAA | |
| | | ggtcatgtgtagatgcgataaatc | GTGGACCACCAGCCTGAGTGCAA | |
| | | aagtgattggtgtgcctgggtctc | AATTCAAGTGCACAGCAGGGAACAAAGTCAGCAAGGAATCCAGTGTC | |
| | | actacaagcagc | GAGCCTGTCAGCTGTCCAGAGAA | |
| | | ctatctgc | AGGTCTGGACATCTATCTCATCATTGGCATATGTGGAGGAGGCAGCC | |
| | | [Seq ID No 53] | TCTTGATGGTCTTTGTGGCACTG | |
| | | | CTCGTTTTCTATATCACCAAAAGGAAAAAACAGAGGAGTCGGAGAAA | |
| | | | TGATGAGGAGCTGGAGACAAGAG | |
| | | | CCCACAGAGTAGCTACTGAAGAAAGGGGCCGGAAGCCCCAACAAATT | |
| | | | CCAGCTTCAACCCCTCAGAATCC | |
| | | | AGCAACTTCCCAACATCCTCCTCCACCACCTGGTCATCGTTCCCAGG | |
| | | | CACCTAGTCATCGTCCCCCGCCT | |
| | | | CCTGGACACCGTGTTCAGCACCAGCCTCAGAAGAGGCCTCCTGCTCC | |
| | | | GTCGGGCACACAAGTTCACCAGC | |
| | | | AGAAAGGCCCGCCCCTCCCCAGACCTCGAGTTCAGCCAAAACCTCCC | |
| | | | CATGGGGCAGCAGAAAACTCATT | |
| | | | GTCCCCTTCCTCTAATTAAAAAAGATAGAAACTGTCTTTTTCAATAA | |
| | | | AAAGCACTGTGGATTTCTGCCCT | |
| | | | CCTGATGTGCATATCCGTACTTCCATGAGGTGTTTTCTGTGTGCAGA | |
| | | | ACATTGTCACCTCCTGAGGCTGT | |
| | | | GGGCCACAGCCACCTCTGCATCTTCGAACTCAGCCATGTGGTCAACA | |
| | | | TCTGGAGTTTTTGGTCTCCTCAG | |
| | | | AGAGCTCCATCACACCAGTAAGGAGAAGCAATATAAGTGTGATTGCA | |
| | | | AGAATGGTAGAGGACCGAGCACA | |
| | | | GAAATCTTAGAGATTTCTTGTCCCCTCTCAGGTCATGTGTAGATGCG | |
| | | | ATAAATCAAGTGATTGGTGTGCC | |
| | | | TGGGTCTCACTACAAGCAGCCTATCTGCTTAAGAGACTCTGGAGTTT | |
| | | | CTTATGTGCCCTGGTGGACACTT | |
| | | | GCCCACCATCCTGTGAGTAAAAGTGAAATAAAAGCTTTGACTAGAAA | |
| | | | AAAAAAAAAAAAAAAAAAAAAAA | |
| | | | AAAAAAAAAAAAAAAAAAAAAAAAAAAAAAAAAAAAAAA | |
| 205890_ | ubiquitin D | Gatcttaaagccacggagaagcct | >gi\|50355987\|ref\|NM_006398.2\|Homo sapiens | 30.29 |
| s_at | | ctcatcttatggcattgacaaaga | ubiquitin D (UBD), mRNA | |
| | | gaagaccatccaccttaccctgaa | GATTGCTTGAGGAGAGAAGTATGTGATCAGAAAGCATTCTTTGTCTA | |
| | | agtggtgaagcccagtgatgagga | TTAACTCCTGCCCAGCAAAAGTG | |
| | | gctgcccttgtttcttgtggagtc | AAAGAAAATTCATGGGAGCATGCAAGAACAAAGAGCACAGCAAAGCT | |
| | | aggtgatgaggcaaagaggcacct | GGACAAACACAGCAATCCAGGCA | |
| | | cctccaggtgcgaaggtccagctc | GGGGATTTCCAACTCAACTCTGGTATGTAAGCTGCATGCAAAGTCCT | |
| | | agtggcacaagtgaaagcaatgat | TTTTCTGTCTCTGGTTTCTGGCC | |
| | | cgagactaagacgggtataatccc | CCTTGTCTGCAGAGATGGCTCCCAATGCTTCCTGCCTCTGTGTGCAT | |
| | | tgagacccagattgtgacttgcaa | GTCCGTTCCGAGGAATGGGATTT | |
| | | tggaaagagactggaagatgggaa | AATGACCTTTGATGCCAACCCATATGACAGCGTGAAAAAAATCAAAG | |
| | | gatgatggcagattacggcatcag | AACATGTCCGGTCTAAGACCAAG | |
| | | aaagggcaacttactcttcctggc | GTTCCTGTGCAGGACCAGGTTCTTTTGCTGGGCTCCAAGATCTTAAA | |
| | | atcttattgtattggagggtgacc | GCCACGGAGAAGCCTCTCATCTT | |
| | | accctggggatggggtgttggcag | ACGGCATTGACAAAGAGAAGACCATCCACCTTACCCTGAAAGTGGTG | |
| | | gggtcaaaaagcttatttctttta | AAGCCCAGTGATGAGGAGCTGCC | |
| | | atctcttactcaacgaacacatct | CTTGTTTCTTGTGGAGTCAGGTGATGAGGCAAAGAGGCACCTCCTCC | |
| | | tctgatgatttcccaaaattaatg | AGGTGCGAAGGTCCAGCTCAGTG | |
| | | agaatgagatgagtagagtaagat | GCACAAGTGAAAGCAATGATCGAGACTAAGACGGGTATAATCCCTGA | |
| | | ttgggtgggatgggtaggatgaag | GACCCAGATTGTGACTTGCAATG | |
| | | tatattgcccaactctatgtttct | GAAAGAGACTGGAAGATGGGAAGATGATGGCAGATTACGGCATCAGA | |
| | | ttga | AAGGGCAACTTACTCTTCCTGGC | |
| | | [Seq ID No 54] | ATCTTATTGTATTGGAGGGTGACCACCCTGGGCATGGGGTGTTGGCA | |
| | | | GGGGTCAAAAAGCTTATTTCTTT | |
| | | | TAATCTCTTACTCAACGAACACATCTTCTGATGATTTCCCAAAATTA | |
| | | | ATGAGAATGAGATGAGTAGAGTA | |
| | | | AGATTTGGGTGGGATGGGTAGGATGAAGTATATTGCCCAACTCTATG | |
| | | | TTTCTTTGATTCTAACACAATTA | |
| | | | ATTAAGTGACATGATTTTTACTAATGTATTACTGAGACTAGTAAATA | |
| | | | AATTTTTAAGCCAA | |
| 206118_ | signal | gctgacatcctgcgagactacaaa | >gi\|21618332\|ref\|NM_003151.2\|Homo sapiens | 13.56 |
| at | transducer | gttattatggctgaaaacattcct | signal transducer and activator of | |
| | and | gaaaaccctctg | transcription 4 (STAT4), mRNA | |
| | activator | aagtacctatatcctgacattccc | AGAGGACGCCCGGTGAAGGGGCTCCAGCCTGGCAGTTTCTGCGTGTT | |
| | of tran- | aaagacaaagccttcggtaaacac | AGCATTTCTAGAATAGAGTGGGT | |
| | scription 4 | tacagct ctcag | GGGAACTGACCCAAGTAAAGTCCCAGAGACTCGAACACTGACGCACA | |
| | | ccttgcgaagtttcaagaccaaca | GGAAAGCCTCAAGTGGGAGGAGA | |
| | | gaaaggggtgacaaaggttatgtt | AATGCAAATCCCCTACTGATGATGGCGTCAGCGGCTTTCTCCTAGGG | |
| | | ccttctgttttt | ACTGTGAGGGGCGCTTCTGACTT | |
| | | atccccatctcaacaatccgaagt | TGGACTTGAGCACTGCCTGGGACCTGTGCTGAGAGAGCGCTAGCATG | |
| | | gattcaacagagccacattctcca | TCTCAGTGGAATCAAGTCCAACA | |
| | | tcagaccttctt | GTTAGAAATCAAGTTTTTGGAGCAGGTGGATCAATTCTATGATGACA | |
| | | cccatgtctccaagtgtgtatgcg | ACTTTCCCATGGAAATTCGGCAT | |
| | | gtgttgagagaaaacctgagtccc | CTGTTGGCCCAATGGATTGAAAATCAAGACTGGGAGGCAGCTTCTAA | |
| | | acaacaattgaa | CAATGAAACCATGGCAACGATTC | |
| | | actgcaatgaagtctccttattct | TTCTTCAAAACTTGTTAATACAACTGGATGAACAGTTAGGTCGTGTT | |
| | | gctgaatgacaggataaactctga | TCCAAAGAGAAAAACCTACTCTT | |
| | | cgcaccaagaaa | GATACACAATCTAAAAAGAATTAGGAAGGTCCTTCAGGGAAAATTTC | |
| | | ggaagcaaatgaaaaagtttaaag | ATGGAAATCCAATGCATGTAGCT | |
| | | actgttctttgcccaataaccaca | GTGGTTATTTCAAACTGTTTAAGGGAAGAGAGGAGAATATTGGCTGC | |
| | | ttttatttcttc | AGCCAACATGCCTGTCCAGGGGC | |
| | | agctttgtaaataccaggttctag | CTCTAGAGAAATCCTTACAAAGTTCTTCAGTTTCAGAAAGACAGAGG | |
| | | gaaatgtttgacatctgaagctct | AATGTGGAGCACAAAGTGGCTGC | |
| | | cttcacactccc | CATTAAAAACAGTGTGCAGATGACAGAACAAGATACCAAATACTTAG | |
| | | gtggcactcctcaattgggag | AAGATCTGCAAGACGAATTTGAC | |
| | | [Seq ID No 55] | TACAGGTATAAAACAATTCAGACAATGGATCAGAGTGACAAGAATAG | |
| | | | TGCCATGGTGAATCAGGAAGTTT | |
| | | | TGACACTGCAGGAAATGCTTAACAGCCTCGATTTCAAGAGAAAGGAG | |
| | | | GCTCTCAGTAAAATGACCCAAAT | |
| | | | CATCCATGAGACAGACCTGTTAATGAACACCATGCTCATAGAAGAGC | |
| | | | TGCAAGACTGGAAGCGGCGGCAG | |
| | | | CAAATCGCCTGCATCGGGGGTCCACTCCACAATGGGCTCGACCAGCT | |
| | | | TCAGAACTGCTTTACACTATTGG | |
| | | | CAGAAAGTCTTTTCCAACTGAGAAGGCAATTGGAGAAACTAGAGGAG | |
| | | | CAATCTACCAAAATGACATATGA | |
| | | | AGGTGATCCCATTCCAATGCAAAGAACTCACATGCTAGAAAGAGTCA | |
| | | | CCTTCTTGATCTACAACCTTTTC | |
| | | | AAGAACTCATTTGTGGTTGAGCGACAGCCATGTATGCCAACCCACCC | |
| | | | TCAGAGGCCGTTGGTACTTAAAA | |
| | | | CCCTAATTCAGTTCACTGTAAAACTAAGGCTACTAATAAAATTGCCA | |
| | | | GAACTAAACTATCAGGTAAAGGT | |
| | | | TAAGGCATCAATTGACAAGAATGTTTCAACTCTAAGCAACCGAAGAT | |
| | | | TTGTACTTTGTGGAACTAATGTC | |
| | | | AAAGCCATGTCTATTGAAGAATCTTCCAATGGGAGTCTCTCAGTAGA | |
| | | | ATTTCGACATTTGCAACCAAAGG | |
| | | | AAATGAAGTCCAGTGCTGGAGGTAAAGGAAATGAGGGCTGTCACATG | |
| | | | GTGACTGAAGAACTTCATTCCAT | |
| | | | AACGTTTGAAACACAGATCTGCCTCTATGGCCTGACCATAGATTTGG | |
| | | | AGACCAGCTCATTGCCTGTGGTG | |
| | | | ATGATTTCCAATGTCAGTCAGTTACCTAATGCTTGGGCATCCATCAT | |
| | | | TTGGTACAACGTGTCAACCAACG | |
| | | | ATTCCCAGAACTTGGTTTTCTTTAATAATCCTCCACCTGCCACATTG | |
| | | | AGTCAACTACTGGAGGTGATGAG | |
| | | | CTGGCAGTTTTCATCGTACGTTGGTCGTGGTCTTAACTCAGATCAAC | |
| | | | TCCATATGCTGGCAGAGAAGCTT | |
| | | | ACAGTCCAATCTAGCTACAGTGATGGTCACCTCACCTGGGCCAAGTT | |
| | | | CTGCAAGGAACATTTACCTGGTA | |
| | | | AATCATTTACCTTTTGGACATGGCTTGAAGCAATATTGGATCTAATT | |
| | | | AAGAAACACATTCTTCCCCTTTG | |
| | | | GATTGATGGGTATGTCATGGGCTTTGTTAGCAAAGAGAAGGAACGGC | |
| | | | TGTTGCTAAAGGATAAAATGCCT | |
| | | | GGCACCTTTTTATTAAGATTCAGTGAAAGCCATCTCGGAGGAATAAC | |
| | | | TTTCACCTGGGTGGACCATTCTG | |
| | | | AAAGTGGGGAAGTGAGATTCCACTCTGTAGAACCCTACAATAAAGGC | |
| | | | CGGTTGTCTGCTCTGCCATTCGC | |
| | | | TGACATCCTGCGAGACTACAAAGTTATTATGGCTGAAAACATTCCTG | |
| | | | AAAACCCTCTGAAGTACCTATAT | |
| | | | CCTGACATTCCCAAAGACAAAGCCTTCGGTAAACACTACAGCTCTCA | |
| | | | GCCTTGCGAAGTTTCAAGACCAA | |
| | | | CAGAAAGGGGTGACAAAGGTTATGTTCCTTCTGTTTTTATCCCCATC | |
| | | | TCAACAATCCGAAGTGATTCAAC | |
| | | | AGAGCCACATTCTCCATCAGACCTTCTTCCCATGTCTCCAAGTGTGT | |
| | | | ATGCGGTGTTGAGAGAAAACCTG | |
| | | | AGTCCCACAACAATTGAAACTGCAATGAAGTCTCCTTATTCTGCTGA | |
| | | | ATGACAGGATAAACTCTGACGCA | |
| | | | CCAAGAAAGGAAGCAAATGAAAAAGTTTAAAGACTGTTCTTTGCCCA | |
| | | | ATAACCACATTTTATTTCTTCAG | |
| | | | CTTTGTAAATACCAGGTTCTAGGAAATGTTTGACATCTGAAGCTCTC | |
| | | | TTCACACTCCCGTGGCACTCCTC | |
| | | | AATTGGGAGTGTTGTGACTGAAATGCTTGAAACCAAAGCTTCAGATA | |
| | | | AACTTGCAAGATAAGACAACTTT | |
| | | | AAGAAACCAGTGTTAATAACAATATTAACAG | |
| 206666_ | granzyme K | aaacctctcttagatctggaacca | >gi\|73747815\|ref\|NM_002104.2\|Homo sapiens | 18.25 |
| at | (granzyme | aatgcaaggttactggctggggag | granzyme K (granzyme 3; tryptase II) (GZMK), | |
| | 3; tryptase | ccaccgatccagattcattaagac | mRNA | |
| | II)/// | cttctgacaccctgcgagaagtca | GATCAACACATTTCATGTGGGCTTCTTAAATCTAAATCTTTAAAATG | |
| | granzyme K | ctgttactgtcctaagtcgaaaac | ACTAAGTTTTCTTCCTTTTCTCT | |
| | (granzyme | tttgcaacagccaaagttactaca | GTTTTTCCTAATAGTTGGGGCTTATATGACTCATGTGTGTTTCAATA | |
| | 3; tryptase | acggcgacccttttatcaccaaag | TGGAAATTATTGGAGGGAAAGAA | |
| | II) | acatggtctgtg | GTGTCACCTCATTCCAGGCCATTTATGGCCTCCATCCAGTATGGCGG | |
| | | caggagatgccaaaggccagaagg | ACATCACGTTTGTGGAGGTGTTC | |
| | | attcctgtaagggtgactcagggg | TGATTGATCCACAGTGGGTGCTGACAGCAGCCCACTGCCAATATCGG | |
| | | gccccttgatctgtaaaggtgtct | TTTACCAAAGGCCAGTCTCCCAC | |
| | | tccacgctatagtctctggaggtc | TGTGGTTTTAGGCGCACACTCTCTCTCAAAGAATGAGGCCTCCAAAC | |
| | | atgaatgtggtgttgccacaaagc | AAACACTGGAGATCAAAAAATTT | |
| | | ctggaatctacaccctgttaacca | ATACCATTCTCAAGAGTTACATCAGATCCTCAATCAAATGATATCAT | |
| | | agaaataccagacttggatcaaaa | GCTGGTTAAGCTTCAAACAGCCG | |
| | | gcaaccttgtcccgcctcatacaa | CAAAACTCAATAAACATGTCAAGATGCTCCACATAAGATCCAAAACC | |
| | | attaagttacaaataattttattg | TCTCTTAGATCTGGAACCAAATG | |
| | | gatgcacttgcttcttttttccta | CAAGGTTACTGGCTGGGGAGCCACCGATCCAGATTCATTAAGACCTT | |
| | | atatgctcgcaggttagagttggg | CTGACACCCTGCGAGAAGTCACT | |
| | | tgtaagtaaagcagagcacatatg | GTTACTGTCCTAAGTCGAAAACTTTGCAACAGCCAAAGTTACTACAA | |
| | | gggtccattttt | CGGCGACCCTTTTATCACCAAAG | |
| | | gcacttgta | ACATGGTCTGTGCAGGAGATGCCAAAGGCCAGAAGGATTCCTGTAAG | |
| | | [Seq ID No 56] | GGTGACTCAGGGGGCCCCTTGAT | |
| | | | CTGTAAAGGTGTCTTCCACGCTATAGTCTCTGGAGGTCATGAATGTG | |
| | | | GTGTTGCCACAAAGCCTGGAATC | |
| | | | TACACCCTGTTAACCAAGAAATACCAGACTTGGATCAAAAGCAACCT | |
| | | | TGTCCCGCCTCATACAAATTAAG | |
| | | | TTACAAATAATTTTATTGGATGCACTTGCTTTTTTTTTCCTAATATG | |
| | | | CTCGCAGGTTAGAGTTGGGTGTA | |
| | | | AGTAAAGCAGAGCACATATGGGGTCCATTTTTGCACTTGTAAGTCAT | |
| | | | TTTATTAAGGAATCAAGTTCTTT | |
| | | | TTCACTTGTATCACTGATGTATTTCTACCATGCTGGTTTTATTCTAA | |
| | | | ATAAAATTTAGAAGACTCAAAAA | |
| | | | AAAAAAAAAAAAAAAAAAAAAAAA | |
| 206804_ | CD3G | gtaatgccaaggaccctcgaggga | >gi\|4557428\|ref\|NM_000073.1\|Homo sapiens CD3g | 23.86 |
| at | antigen | tgtatcagtgtaaaggatcacaga | molecule, gamma (CD3-TCR complex) (CD3G), mRNA | |
| | gamma | acaagtcaaaaccactccaagtgt | GGGCTGCTCCACGCTTTTGCCGGAGACAGAGACTGACATGGAACAGG | |
| | polypeptide | attacagaatgtgtcagaactgca | GGAAGGGCCTGGCTGTCCTCATC | |
| | (TiT3 | ttgaactaaatgcagccaccatat | CTGGCTATCATTCTTCTTCAAGGTACTTTGGCCCAGTCAATCAAAGG | |
| | complex) | ctggctttctctttgctgaaatcg | AAACCACTTGGTTAAGGTGTATG | |
| | | tcagcattttcgtccttgctgttg | ACTATCAAGAAGATGGTTCGGTACTTCTGACTTGTGATGCAGAAGCC | |
| | | gggtctacttca | AAAAATATCACATGGTTTAAAGA | |
| | | ttgctggacaggatggagttcgcc | TGGGAAGATGATCGGCTTCCTAACTGAAGATAAAAAAAAATGGAATC | |
| | | agtcgagagcttcagacaagcaga | TGGGAAGTAATGCCAAGGACCCT | |
| | | ctctgttgcccaatgaccagctct | CGAGGGATGTATCAGTGTAAAGGATCACAGAACAAGTCAAAACCACT | |
| | | accagcccctcaaggatcgagaag | CCAAGTGTATTACAGAATGTGTC | |
| | | atgaccagtacagccaccttcaag | AGAACTGCATTGAACTAAATGCAGCCACCATATCTGGCTTTCTCTTT | |
| | | gaaaccagttgaggaggaattgaa | GCTGAAATCGTCAGCATTTTCGT | |
| | | ctcaggactcagagtagtccaggt | CCTTGCTGTTGGGGTCTACTTCATTGCTGGACAGGATGGAGTTCGCC | |
| | | gttctcctcctattcagttcccag | AGTCGAGAGCTTCAGACAAGCAG | |
| | | aatcaaagcaatgcattttggaaa | ACTCTGTTGCCCAATGACCAGCTCTACCAGCCCCTCAAGGATCGAGA | |
| | | gctcctagcagagagactttcagc | AGATGACCAGTACAGCCACCTTC | |
| | | cctaaatctagactcaaggttccc | AAGGAAACCAGTTGAGGAGGAATTGAACTCAGGACTCAGAGTAGTCC | |
| | | agagatgac | AGGTGTTCTCCTCCTATTCAGTT | |
| | | [Seq ID No 57] | CCCAGAATGAAAGCAATGCATTTTGGAAAGCTCCTAGCAGAGAGACT | |
| | | | TTCAGCCCTAAATCTAGACTCAA | |
| | | | GGTTCCCAGAGATGACAAATGGAGAAGAAAGGCCATCAGAGCAAATT | |
| | | | TGGGGGTTTCTCAAATAAAATAA | |
| | | | AAATAAAAACAAATACTCTGTTTCAGAAGCGCCACCTATTGGGGAAA | |
| | | | ATTGT | |
| 207651_ | G protein- | ttgccttgtaattcgacagctcta | >gi\|31377771\|ref\|NM_013308.2\|Homo sapiens G | 46.65 |
| at | coupled | cagaaacaaagataatgaaaatta | protein-coupled receptor 171 (GPR171), mRNA | |
| | receptor | cccaaatgtgaa | GGTTGCTACTGCTGCGGCTAACCAAACAGCTCATGCTTCTCTGAAGA | |
| | 171 | aaaggctctcatcaacatactttt | CTTGCAGCAAGGTTTGCTGAGGC | |
| | | agtgaccacgggctacatcatatg | TCACAGAAGATAGCCCCAGTGTTTTGGAGTGGTTTTGAATGTGATTC | |
| | | ctttgttcctta | TGAGATCAGACTGACTGAGCTGG | |
| | | ccacattgtccgaatcccgtatac | AATCCTGGCTTTATATCTTACCAGCTACACAACCTTGGAGTCTTAGA | |
| | | cctcagccagacagaagtcataac | AATTTTTTCTTTTCAATAAGCAG | |
| | | tgattgctcaac | TCATCCTTACTTTCCCTCAAGATGACAAACAGTTCGTTCTTCTGCCC | |
| | | caggatttcactcttcaaagccaa | AGTTTATAAAGATCTGGAGCCAT | |
| | | agaggctacactgctcctggctgt | TCACGTATTTTTTTTTTTTAGTTTTCCTTGTTGGAATTATTGGAAGT | |
| | | gtcgaacctgtg | TGTTTTGCAACCTGGGCTTTTAT | |
| | | ctttgatcctatcctgtactatca | ACAGAAGAATACGAATCACAGGTGTGTGAGCATCTACTTAATTAATT | |
| | | cctctcaaaagcattccgctcaaa | TGCTTACAGCCGATTTCCTGCTT | |
| | | ggtcactgagac | ACTCTGGCATTACCAGTGAAAATTGTTGTTGACTTGGGTGTGGCACC | |
| | | ttttgcctcacctaaagagaccaa | TTGGAAGCTGAAGATATTCCACT | |
| | | ggctcagaaagaaaaattaagatg | GCCAAGTAACAGCCTGCCTCATCTATATCAATATGTATTTATCAATT | |
| | | tgaaaataatgc | ATCTTCTTAGCATTTGTCAGCAT | |
| | | ataaaagacaggattttttgtgct | TGACCGCTGTCTTCAGCTGACACACAGCTGCAAGATCTACCGAATAC | |
| | | accaattctggccttactgga | AAGAACCCGGATTTGCCAAAATG | |
| | | [Seq ID No 58] | ATATCAACCGTTGTGTGGCTAATGGTCCTTCTTATAATGGTGCCAAA | |
| | | | TATGATGATTCCCATCAAAGACA | |
| | | | TCAAGGAAAAGTCAAATGTGGGTTGTATGGAGTTTAAAAAGGAATTT | |
| | | | GGAAGAAATTGGCATTTGCTGAC | |
| | | | AAATTTCATATGTGTAGCAATATTTTTAAATTTCTCAGCCATCATTT | |
| | | | TAATATCCAATTGCCTTGTAATT | |
| | | | CGACAGCTCTACAGAAACAAAGATAATGAAAATTACCCAAATGTGAA | |
| | | | AAAGGCTCTCATCAACATACTTT | |
| | | | TAGTGACCACGGGCTACATCATATGCTTTGTTCCTTACCACATTGTC | |
| | | | CGAATCCCGTATACCCTCAGCCA | |
| | | | GACAGAAGTCATAACTGATTGCTCAACCAGGATTTCACTCTTCAAAG | |
| | | | CCAAAGAGGCTACACTGCTCCTG | |
| | | | GCTGTGTCGAACCTGTGCTTTGATCCTGTCCTGTACTATCACCTCTC | |
| | | | AAAAGCATTCCGCTCAAAGGTCA | |
| | | | CTGAGACTTTTGCCTCACCTAAAGAGACCAAGGCTCAGAAAGAAAAA | |
| | | | TTAAGATGTGAAAATAATGCATA | |
| | | | AAAGACAGGATTTTTGTGCTACCAATTCTGGCCTTACTGGACCATAA | |
| | | | AGTTAATTATAGCTTTGAAAGAT | |
| | | | AAAAAAAAAAAAAAAAACAAAAAAAAACTCAGTATGAAAAAATACAG | |
| | | | TTAGCTAGCAAATATGGACAGGT | |
| | | | TTACTTAGAAATCCTGTTTCTAAATGCAAGTCAAGCTTTATTGTTAG | |
| | | | GCTTGCTGCTACTCATTAACCCA | |
| | | | AATATTTGTACAAAAAACTAAAGAGTCTCATTGAACGAATGTAAAAT | |
| | | | CCTGCAATATCCTTGAAATCCAA | |
| | | | AAGAGGTCCATGACATAGACCCAAAGGTATTCATGAGTTATTCATTT | |
| | | | AAATGCCTGGAACTGACTTCTTG | |
| | | | ATAAAAATATAAAAAATAATTTCCATGTAAGTTACCAGAAAGCCCAC | |
| | | | CAGCAACATAATTTTAAAGCCTT | |
| | | | TCGGATTACTTTTAAAAAATGCAGCTTACATATAACAACTTGTGCCT | |
| | | | ATTTTATTTCTAATCTATCACTT | |
| | | | CAAAAGATGGTAATCTTTCAACTCATTATTCCTCCAATTTTTAATGT | |
| | | | CGAATTTTTTTCTAACACAATAA | |
| | | | CCAAAAGCTTTTATTTATAAAAAGGCTTGAAAAATATAAAAAAAAAA | |
| | | | AAAAAAAAAAAAAAAAAAAAAAA | |
| | | | AAAAAAAAAAAAAAAAAAAAAAAAAAAAAAAAAAA | |
| 207957_ | Protein | tgatgagctttcctttgatccgga | >gi\|89353296\|ref\|NM_001183.4\|Homo sapiens | 34.17 |
| s_at | kinase C, | cgacgtaatcactgacattgagat | ATPase, H+ transporting, lysosomal accessory | |
| | beta 1 | ggtggacgaggg | protein 1 (ATP6AP1), mRNA | |
| | | ctggtggcggggacgttgccatgg | GGGGGCAACGGTCACCTGATCTGCGGCTGTCGAGGCCGCTGAGGCAG | |
| | | ccactttggactcttccctgcaaa | TGGAGGCTGAGGCTATGATGGCG | |
| | | ttatgtcaagct | GCCATGGCGACGGCTCGAGTGCGGATGGGGCCGCGATGCGCCCAGGC | |
| | | tctggagtgactagagctcactgt | GCTCTGGCGCATGCCGTGGCTGC | |
| | | ctactgcaactgtgatttcccatg | CGGTGTTTTTGTCGTTGGCGGCGGCGGCGGCGGCGGCAGCGGCGGAG | |
| | | tccaaagtggct | CAGCAGGTCCCGCTGGTGCTGTG | |
| | | ctgctccaccccctccctattcct | GTCGAGTGACCGGGACTTGTGGGCTCCTGCGGCCGACACTCATGAAG | |
| | | gatgcaaatgtctaaccagatgag | GCCACATCACCAGCGACTTGCAG | |
| | | tttctggacaga | CTCTCTACCTACTTAGATCCCGCCCTGGAGCTGGGTCCCAGGAATGT | |
| | | cttccctctcctgcttcattaagg | GCTGCTGTTCCTGCAGGACAAGC | |
| | | gcttggggcagagacagcatgggg | TGAGCATTGAGGATTTCACAGCATATGGCGGTGTGTTTGGAAACAAG | |
| | | aaggaggtccccttccccaagagt | CAGGACAGCGCCTTTTCTAACCT | |
| | | cctctctatcctggatgagctcat | AGAGAATGCCCTGGACCTGGCCCCCTCCTCACTGGTGCTTCCTGCCG | |
| | | gaacatttctcttgtgttcctgac | TCGACTGGTATGCAGTCAGCACT | |
| | | tccttcccaatgaacacctctctg | CTGACCACTTACCTGCAGGAGAAGCTCGGGGCCAGCCCCTTGCATGT | |
| | | ccaccccaagctctgctctcctcc | GGACCTGGCCACCCTGCGGGAGC | |
| | | tctgtgagctct | TGAAGCTCAATGCCAGCCTCCCTGCTCTGCTGCTCATTCGCCTGCCC | |
| | | gggcttcccagtttgtttacccgg | TACACAGCCAGCTCTGGTCTGAT | |
| | | gaaagtacgtctagattgtgtggt | GGCACCCAGGGAAGTCCTCACAGGCAACGATGAGGTCATCGGGCAGG | |
| | | ttgcctcattgt | TCCTGAGCACACTCAAGTCCGAA | |
| | | gctatttgcccactttccttccct | GATGTCCCATACACAGCGGCCCTCACAGCGGTCCGCCCTTCCAGGGT | |
| | | gaagaaatatctgtgaaccttctt | GGCCCGTGATGTAGCCGTGGTGG | |
| | | tctgttcagtcc | CCGGAGGGCTAGGTCGCCAGCTGCTACAAAAACAGCCAGTATCACCT | |
| | | ta | GTGATCCATCCTCCTGTGAGTTA | |
| | | [Seq Id No 59] | CAATGACACCGCTCCCCGGATCCTGTTCTGGGCCCAAAACTTCTCTG | |
| | | | TGGCGTACAAGGACCAGTGGGAG | |
| | | | GACCTGACTCCCCTCACCTTTGGGGTGCAGGAACTCAACCTGACTGG | |
| | | | CTCCTTCTGGAATGACTCCTTTG | |
| | | | CCAGGCTCTCACTGACCTATGAACGACTCTTTGGTACCACAGTGACA | |
| | | | TTCAAGTTCATTCTGGCCAACCG | |
| | | | CCTCTACCCAGTGTCTGCCCGGCACTGGTTTACCATGGAGCGCCTCG | |
| | | | AAGTCCACAGCAATGGCTCCGTC | |
| | | | GCCTACTTCAATGCTTCCCAGGTCACAGGGCCCAGCATCTACTCCTT | |
| | | | CCACTGCGAGTATGTCAGCAGCC | |
| | | | TGAGCAAGAAGGGTAGTCTCCTCGTGGCCCGCACGCAGCCCTCTCCC | |
| | | | TGGCAGATGATGCTTCAGGACTT | |
| | | | CCAGATCCAGGCTTTCAACGTAATGGGGGAGCAGTTCTCCTACGCCA | |
| | | | GCGACTGTGCCAGCTTCTTCTCC | |
| | | | CCCGGCATCTGGATGGGGCTGCTCACCTCCCTGTTCATGCTCTTCAT | |
| | | | CTTCACCTATGGCCTGCACATGA | |
| | | | TCCTCAGCCTCAAGACCATGGATCGCTTTGATGACCACAAGGGCCCC | |
| | | | AGTATTTCTTTGACCCAGATTGT | |
| | | | GTGACCCTGTGCCAGTGGGGGGGTTGAGGGTGGGACGGTGTCCGTGT | |
| | | | TGTTGCTTTCCCACCCTGCAGCG | |
| | | | CACTGGACTGAAGAGCTTCCCTCTTCCTACTGCAGCATGAACTGCAA | |
| | | | GCTCCCCTCAGCCCATCTTGCTC | |
| | | | CCTCTTCAGCCCGCTGAGGAGCTTTCTTGGGCTGCCCCCATCTCTCC | |
| | | | CAACAAGGTGTACATATTCTGCG | |
| | | | TAGATGCTAGACCAACCAGCTTCCCAGGGTTCGTCGCTGTGAGGCGT | |
| | | | AAGGGACATGAATTCTAGGGTCT | |
| | | | CCTTTCTCCTTATTTATTCTTGTGGCTACATCATCCCTGGCTGTGGA | |
| | | | TAGTGCTTTTGTGTAGCAAATGC | |
| | | | TCCCTCCTTAAGGTTATAGGGCTCCCTGAGTTTGGGAGTGTGGAAGT | |
| | | | ACTACTTAACTGTCTGTCCTGCT | |
| | | | TGGCTGTCGTTATCGTTTTCTGGTGATGTTGTGCTAACAATAAGAAG | |
| | | | TACACGGGTTTATTTCTGTGGCC | |
| | | | TGAGAAGGAAGGGACCTCCACGACAGGTGGGCTGGGTGCGATCGCCG | |
| | | | GCTGTTTGGCATGTTCCCACCGG | |
| | | | GAGTGCCGGGCAGGAGCATGGGGTGCTTGGTTGTTTCCTTCCTAATA | |
| | | | AAATAAACGCGGGTCGCCATGCA | |
| | | | AAAAAAAAA | |
| 208894_ | major histo- | cgatcaccaatgtacctccagagg | >gi\|52426773\|ref\|NM_09111.3\|Homo sapiens | 6.11 |
| at | compatibility | taactgtgctcacgaacagccctg | major histocompatibility complex, class II, DR | |
| | complex, | tggaactgagagagcccaacgtcc | alpha (HLA-DRA), mRNA | |
| | class II, | tcatctgtttcatagacaagttca | ACATTCTCTTTTCTTTTATTCTTGTCTGTTCTGCCTCACTCCCGAGC | |
| | DR alpha/// | cccca | TCTACTGACTCCCAACAGAGCGC | |
| | major histo- | [Seq Id No 60] | CCAAGAAGAAAATGGCCATAAGTGGAGTCCCTGTGCTAGGATTTTTC | |
| | compatibility | | ATCATAGCTGTGCTGATGAGCGC | |
| | complex, | | TCAGGAATCATGGGCTATCAAAGAAGAACATGTGATCATCCAGGCCG | |
| | class II, | | AGTTCTATCTGAATCCTGACCAA | |
| | DR alpha | | TCAGGCGAGTTTATGTTTGACTTTGATGGTGATGAGATTTTCCATGT | |
| | | | GGATATGGCAAAGAAGGAGACGG | |
| | | | TCTGGCGGCTTGAAGAATTTGGACGATTTGCCAGCTTTGAGGCTCAA | |
| | | | GGTGCATTGGCCAACATAGCTGT | |
| | | | GGACAAAGCCAACCTGGAAATCATGACAAAGCGCTCCAACTATACTC | |
| | | | CGATCACCAATGTACCTCCAGAG | |
| | | | GTAACTGTGCTCACGAACAGCCCTGTGGAACTGAGAGAGCCCAACGT | |
| | | | CCTCATCTGTTTCATAGACAAGT | |
| | | | TCACCCCACCAGTGGTCAATGTCACGTGGCTTCGAAATGGAAAACCT | |
| | | | GTCACCACAGGAGTGTCAGAGAC | |
| | | | AGTCTTCCTGCCCAGGGAAGACCACCTTTTCCGCAAGTTCCACTATC | |
| | | | TCCCCTTCCTGCCCTCAACTGAG | |
| | | | GACGTTTACGACTGCAGGGTGGAGCACTGGGGCTTGGATGAGCCTCT | |
| | | | TCTCAAGCACTGGGAGTTTGATG | |
| | | | CTCCAAGCCCTCTCCCAGAGACTACAGAGAACGTGGTGTGTGCCCTG | |
| | | | GGCCTGACTGTGGGTCTGGTGGG | |
| | | | CATCATTATTGGGACCATCTTCATCATCAAGGGATTGCGCAAAAGCA | |
| | | | ATGCAGCAGAACGCAGGGGGCCT | |
| | | | CTGTAAGGCACATGGAGGTGATGGTGTTTCTTAGAGAGAAGATCACT | |
| | | | GAAGAAACTTCTGCTTTAATGGC | |
| | | | TTTACAAAGCTGGCAATATTACAATCCTTGACCTCAGTGAAAGCAGT | |
| | | | CATCTTCAGCATTTTCCAGCCCT | |
| | | | ATAGCCACCCCAAGTGTGGATATGCCTCTTCGATTGCTCCGTACTCT | |
| | | | AACATCTAGCTGGCTTCCCTGTC | |
| | | | TATTGCCTTTTCCTGTATCTATTTTCCTCTATTTCCTATCATTTTAT | |
| | | | TATCACCATGCAATGCCTCTGGA | |
| | | | ATAAAACATACAGGAGTCTGTCTCTGCTATGGAATGCCCCATGGGGC | |
| | | | ATCTCTTGTGTACTTATTGTTTA | |
| | | | AGGTTTCCTCAAACTGTGATTTTTCTGAACACAATAAACTATTTTGA | |
| | | | TGATCTTGGGTGGAAAAAAAAAA | |
| | | | AAAAAAA | |
| 209480_ | Major histo- | aaaaaaggaaatcgctgcagaatg | >gi\|24797068\|ref\|NM_002123.2\|Homo sapiens | 12.55 |
| at | compatibility | aaggaatatcccttgaggtgaccc | major histocompatibility complex, class II, DQ | |
| | complex, | agccaacctgtggccagaaggagg | beta 1 (HLA-DQB1), mRNA | |
| | class II, | gttgtacct:gaaaagaccactga | CAGATCCATCAGGTCCGAGCTGTGTTGACTACCACTTTTCCCTTCGT | |
| | DQ beta 1/// | aagcattttggggtgtcaagtaag | CTCAATTATGTCTTGGAAAAAGG | |
| | Major histo- | ggtgggcagaggaggtagaaaatc | CTTTGCGGATCCCCGGAGGCCTTCGGGCAGCAACTGTGACCTTGATG | |
| | compatibility | aattcaattgtcgcatcattcatg | CTGTCGATGCTGAGCACCCCAGT | |
| | complex, | gttctttaatattgatgctcagtg | GGCTGAGGGCAGAGACTCTCCCGAGGATTTCGTGTACCAGTTTAAGG | |
| | class II, | cattggccttagaatatcccagcc | GCATGTGCTACTTCACCAACGGG | |
| | DQ beta 1 | tctcttctggtttggtgagtgctg | ACAGAGCGCGTGCGTCTTGTGAGCAGAAGCATCTATAACCGAGAAGA | |
| | | tgtaagtaagcatggtagaattgt | GATCGTGCGCTTCGACAGCGACG | |
| | | ttggagacatatatagtgatcctt | TGGGGGAGTTCCGGGCGGTGACGCTGCTGGGGCTGCCTGCCGCCGAG | |
| | | ggtcactggtgt | TACTGGAACAGCCAGAAGGACAT | |
| | | ttcaaacattctggaaagtcacat | CCTGGAGAGGAAACGGGCGGCGGTGGACAGGGTGTGCAGACACAACT | |
| | | cgatcaagaatatttttt | ACCAGTTGGAGCTCCGCACGACC | |
| | | [Seq Id No 61] | TTGCAGCGGCGAGTGGAGCCCACAGTGACCATCTCCCCATCCAGGAC | |
| | | | AGAGGCCCTCAACCACCACAACC | |
| | | | TGCTGGTCTGCTCGGTGACAGATTTCTATCCAGCCCAGATCAAAGTC | |
| | | | CGGTGGTTTCGGAATGACCAGGA | |
| | | | GGAGACAGCTGGCGTTGTGTCCACCCCCCTTATTAGGAATGGTGACT | |
| | | | GGACCTTCCAGATCCTGGTGATG | |
| | | | CTGGAAATGACTCCCCAGCGTGGAGACGTCTACACCTGCCACGTGGA | |
| | | | GCACCCCAGCCTCCAGAGCCCCA | |
| | | | TCACCGTGGAGTGGCGGGCTCAATCTGAATCTGCCCAGAGCAAGATG | |
| | | | CTGAGTGGCATTGGAGGCTTCGT | |
| | | | GCTGGGGCTGATCTTCCTCGGGCTGGGCCTTATCATCCATCACAGGA | |
| | | | GTCAGAAAGGGCTCCTGCACTGA | |
| | | | CTCCTGAGACTATTTTAACTGGGATTGGTTATCACTTTTCTGTAACG | |
| | | | CCTGCTTGTCCCTGCCCAGAATT | |
| | | | CCCAGCTGTCTGTGTCAGCCTGTCCCCCTGAGATCAGAGTCCTACAG | |
| | | | TGGCTGTCACGCAGCCACCAGGT | |
| | | | CATCTCCTTTCATCCCCACCTTGAGGCGGATGGCTGTGACCCTACTT | |
| | | | CCTGCACTGACCCACAGCCTCTG | |
| | | | CCTGTGCACGGCCAGCTGCATCTACTCAGGCCCCAAGGGGTTTCTGT | |
| | | | TTCTATTCTCTCCTCAGACTGCT | |
| | | | CAAGAGAAGCACATGAAAACCATTACCTGACTTTAGAGCTTTTTTAC | |
| | | | ATAATTAAACATGATCCTGAGTT | |
| 209613_ | alcohol | gcagatttcttgcttcatatgaca | >gi\|34577060\|ref\|NM_000668.3\|Homo sapiens | 58.89 |
| s_at | dehydrogenase | aagcctcaattactaattgtaaaa | alcohol dehydrogenase IB (class I), beta | |
| | IB | actgaactattc | polypeptide (ADH1B), mRNA | |
| | (class I), | ccagaatcatgttcaaaaaatctg | ATGCACTCAAGCAGAGAAGAAATCCACAAAGACTCACAGTCTGCTGG | |
| | beta poly- | taatttttgctgatcgaaagtgct | TGGGCAGAGAAGACAGAAACGAC | |
| | peptide | tcattgactaaa | ATGAGCACAGCAGGAAAAGTAATCAAATGCAAAGCAGCTGTGCTATG | |
| | | cagtattagtttgtggctataaat | GGAGGTAAAGAAACCCTTTTCCA | |
| | | gattatttagatgatgactgaaaa | TTGAGGATGTGGAGGTTGCACCTCCTAAGGCTTATGAAGTTCGCATT | |
| | | tgtgtataaagt | AAGATGGTGGCTGTAGGAATCTG | |
| | | aattaaaagtaatatggtggcttt | TCACACAGATGACCACGTGGTTAGTGGCAACCTGGTGACCCCCCTTC | |
| | | aagtgtagagatgggatggcaaat | CTGTGATTTTAGGCCATGAGGCA | |
| | | gctgtgaatgca | GCCGGCATCGTGGAGAGTGTTGGAGAAGGGGTGACTACAGTCAAACC | |
| | | gaatgtaaaattggtaactaagaa | AGGTGATAAAGTCATCCCGCTCT | |
| | | atggcacaaacaccttaagcaata | TTACTCCTCAGTGTGGAAAATGCAGAGTTTGTAAAAACCCGGAGAGC | |
| | | tattttcctagt | AACTACTGCTTGAAAAATGATCT | |
| | | agatatatatatacacatacatat | AGGCAATCCTCGGGGGACCCTGCAGGATGGCACCAGGAGGTTCACCT | |
| | | atacacatatacaaatgtatattt | GCAGGGGGAAGCCCATTCACCAC | |
| | | ttgcaaaattgt | TTCCTTGGCACCAGCACCTTCTCCCAGTACACGGTGGTGGATGAGAA | |
| | | tttcaatctagaacttttctatta | TGCAGTGGCCAAAATTGATGCAG | |
| | | actaccatgtcttaaaatcaagtc | CCTCGCCCCTGGAGAAAGTCTGCCTCATTGGCTGTGGATTCTCGACT | |
| | | tataatcctagc | GGTTATGGGTCTGCAGTTAACGT | |
| | | attagtttaatattttgaatatgt | TGCCAAGGTCACCCCAGGCTCTACCTGTGCTGTGTTTGGCCTGGGAG | |
| | | aaacacctgtgttaatgctttgtt | GGGTCGGCCTATCTGCTGTTATG | |
| | | aatgcttttccc | GGCTGTAAAGCAGCTGGAGCAGCCAGAATCATTGCGGTGGACATCAA | |
| | | actctcatttgttaatgctttccc | CAAGGACAAATTTGCAAAGGCCA | |
| | | actctcgggaaggatttgcatttt | AAGAGTTGGGTGCCACTGAATGCATCAACCCTCAAGACTACAAGAAA | |
| | | gagctttatctc | CCCATCCAGGAAGTGCTAAAGGA | |
| | | taaatgtgacatgca | AATGACTGATGGAGGTGTGGATTTTTCGTTTGAAGTCATCGGTCGGC | |
| | | [Seq Id No 62] | TTGACACCATGATGGCTTCCCTG | |
| | | | TTATGTTGTCATGAGGCATGTGGCACAAGCGTCATCGTAGGGGTACC | |
| | | | TCCTGCTTCCCAGAACCTCTCAA | |
| | | | TAAACCCTATGCTGCTACTGACTGGACGCACCTGGAAGGGGGCTGTT | |
| | | | TATGGTGGCTTTAAGAGTAAAGA | |
| | | | AGGTATCCCAAAACTTGTGGCTGATTTTATGGCTAAGAAGTTTTCAC | |
| | | | TGGATGCGTTAATAACCCATGTT | |
| | | | TTACCTTTTGAAAAAATAAATGAAGGATTTGACCTGCTTCACTCTGG | |
| | | | GAAAAGTATCCGTACCGTCCTGA | |
| | | | CGTTTTGAGGCAATAGAGATGCCTTCCCCTGTAGCAGTCTTCAGCCT | |
| | | | CCTCTACCCTACAAGATCTGGAG | |
| | | | CAACAGCTAGGAAATATCATTAATTCAGCTCTTCAGAGATGTTATCA | |
| | | | ATAAATTACACATGGGGGCTTTC | |
| | | | CAAAGAAATGGAAATTGATGGGAAATTATTTTTCAGGAAAATTTAAA | |
| | | | ATTCAAGTGAGAAGTAAATAAAG | |
| | | | TGTTGAACATCAGCTGGGGAATTGAAGCCAACAAACCTTCCTTCTTA | |
| | | | ACCATTCTACTGTGTCACCTTTG | |
| | | | CCATTGAGGAAAAATATTCCTGTGACTTCTTGCATTTTTGGTATCTT | |
| | | | CATAATCTTTAGTCATCGAATCC | |
| | | | CAGTGGAGGGGACCCTTTTACTTGCCCTGAACATACACATGCTGGGC | |
| | | | CATTGTGATTGAAGTCTTCTAAC | |
| | | | TCTGTCTCAGTTTTCACTGTCGACATTTTCCTTTTTCTAATAAAAAT | |
| | | | GTACCAAATCCCTGGGGTAAAAG | |
| | | | CTAGGGTAAGGTAAAGGATAGACTCACATTTACAAGTAGTGAAGGTC | |
| | | | CAAGAGTTCTAAATACAGGAAAT | |
| | | | TTCTTAGGAACTCAAATAAAATGCCCCACATTTTACTACAGTAAATG | |
| | | | GCAGTGTTTTTATGACTTTTATA | |
| | | | CTATTTCTTTATGGTCGATATACAATTGATTTTTTAAATAATAGCA | |
| | | | GATTTCTTGCTTCATATGACAAA | |
| | | | GCCTCAATTACTAATTGTAAAAACTGAACTATTCCCAGAATCATGTT | |
| | | | CAAAAAATCTGTAATTTTTGCTG | |
| | | | ATGAAAGTGCTTCATTGACTAAACAGTATTAGTTTGTGGCTATAAAT | |
| | | | GATTATTTAGATGATGACTGAAA | |
| | | | ATGTGTATAAAGTAATTAAAAGTAATATGGTGGCTTTAAGTGTAGAG | |
| | | | ATGGGATGGCAAATGCTGTGAAT | |
| | | | GCAGAATGTAAAATTGGTAACTAAGAAATGGCACAAACACCTTAAGC | |
| | | | AATATATTTTCCTAGTAGATATA | |
| | | | TATATACACATACATATATACACATATACAAATGTATATTTTTGCAA | |
| | | | AATTGTTTTCAATCTAGAACTTT | |
| | | | TCTATTAACTACCATGTCTTAAAATCAAGTCTATAATCCTAGCATTA | |
| | | | GTTTAATATTTTGAATATGTAAA | |
| | | | GACCTGTGTTAATGCTTTGTTAATGCTTTTCCCACTCTCATTTGTTA | |
| | | | ATGCTTTCCCACTCTCAGGGGAA | |
| | | | GGATTTGCATTTTGAGCTTTATCTCTAAATGTGACATGCAAAGATTA | |
| | | | TTCCTGGTAAAGGAGGTAGCTGT | |
| | | | CTCCAAAAATGCTATTGTTGCAATATCTACATTCTATTTCATATTAT | |
| | | | GAAAGACCTTAGACATAAAGTAA | |
| | | | AATAGTTTATCATTTACTGTGTGATCTTCAGTAAGTCTCTCAGGCTC | |
| | | | TCTGAGCTTGTTCATCCTTTGTT | |
| | | | TTGAAAAAATTACTCAACCAATCCATTACAGCTTAACCAAGATTAAA | |
| | | | TGGGATGATGTTAATGAAAGAGC | |
| | | | TTCGCC | |
| 209670_ | T cell | caactggatcctacccgaatttat | >gi\|36944\|emb\|X02592.1\|HSTCRAR Human mRNA for | 24.26 |
| at | receptor | gattaagattgctgaagagctgcc | receptor alpha chain (TCR-alpha) | |
| | alpha | aaacactgctgc | TTTTGAAACCCTTCAAAGGCAGAGACTTGTCCAGCCTAACCTGCCTG | |
| | constant/// | caccccctctgttcccttattgct | CTGCTCCTAGCTCCTGAGGCTCA | |
| | T cell | gcttgtcactgcctgacattcacg | GGGCCCTTGGCTTCTGTCCGCTCTGCTCAGGGCCCTCCAGCGTGGCC | |
| | receptor | gcagaggcaagg | ACTGCTCAGCCATGCTCCTGCTG | |
| | alpha | ctgctgcagcctcccctggctgtg | CTCGTCCCAGTGCTCGAGGTGATTTTTACCCTGGGAGGAACCAGAGC | |
| | constant | cacattccctcctgctccccagag | CCAGTCGGTGACCCAGCTTGGCA | |
| | | actgcctccgcc | GCCACGTCTCTGTCTCTGAAGGAGCCCTGGTTCTGCTGAGGTGCAAC | |
| | | atcccacagatgatggatcttcag | TACTCATCGTCTGTTCCACCATA | |
| | | tgggttctcttgggctctaggtcc | TCTCTTCTGGTATGTGCAATACCCCAACCAAGGACTCCAGCTTCTCC | |
| | | tggagaatgttg | TGAAGTACACATCAGCGGCCACC | |
| | | tgaggggtttatttttttttaata | CTGGTTAAAGGCATCAACGGTTTTGAGGCTGAATTTAAGAAGAGTGA | |
| | | gtgttcataaagaaatacatagta | AACCTCCTTCCACCTGACGAAAC | |
| | | ttcttcttctca | CCTCAGCCCATATGAGCGACGCGGCTGAGTACTTCTGTGCTGTGAGT | |
| | | agacgtggggggaaattatctcat | GATCTCGAACCGAACAGCAGTGC | |
| | | tatcgaggccctgctatgctgtgt | TTCCAAGATAATCTTTGGATCAGGGACCAGACTCAGCATCCGGCCAA | |
| | | gtctgggcgtgt | ATATCCAGAACCCTGACCCTGCC | |
| | | tgtatgtcctgc | GTGTACCAGCTGAGAGACTCTAAATCCAGTGACAAGTCTGTCTGCCT | |
| | | [Seq Id No 63] | ATTCACCGATTTTGATTCTCAAA | |
| | | | CAAATGTGTCACAAAGTAAGGATTCTGATGTGTATATCACAGACAAA | |
| | | | ACTGTGCTAGACATGAGGTCTAT | |
| | | | GGACTTCAAGAGCAACAGTGCTGTGGCCTGGAGCAACAAATCTGACT | |
| | | | TTGCATGTGCAAACGCCTTCAAC | |
| | | | AACAGCATTATTCCAGAAGACACCTTCTTCCCCAGCCCAGAAAGTTC | |
| | | | CTGTGATGTCAAGCTGGTCGAGA | |
| | | | AAAGCTTTGAAACAGATACGAACCTAAACTTTCAAAACCTGTCAGTG | |
| | | | ATTGGGTTCCGAATCCTCCTCCT | |
| | | | GAAAGTGGCCGGGTTTAATCTGCTCATGACGCTGCGGCTGTGGTCCA | |
| | | | GCTGAGATCTGCAAGATTGTAAG | |
| | | | ACAGCCTGTGCTCCCTCGCTCCTTCCTCTGCATTGCCCCTCTTCTCC | |
| | | | CTCTCCAAACAGAGGGAACTCTC | |
| | | | CTACCCCCAAGGAGGTGAAAGCTGCTACCACCTCTGTGCCCCCCCGG | |
| | | | TAATGCCACCAACTGGATCCTAC | |
| | | | CCGAATTTATGATTAAGATTGCTGAAGAGCTGCCAAACACTGCTGCC | |
| | | | ACCCCCTCTGTTCCCTTATTGCT | |
| | | | GCTTGTCACTGCCTGACATTCACGGCAGAGGCAAGGCTGCTGCAGCC | |
| | | | TCCCCTGGCTGTGCACATTCCCT | |
| | | | CCTGCTCCCCAGAGACTGCCTCCGCCATCCCACAGATGATGGATCTT | |
| | | | CAGTGGGTTCTCTTGGGCTCTAG | |
| | | | GTCCTGGAGAATGTTGTGAGGGGTTTATTTTTTTTTAATAGTGTTCA | |
| | | | TAAAGAAATACATAGTATTCTTC | |
| | | | TTCTCAAGACGTGGGGGGAAATTATCTCATTATCGAGGCCCTGCTAT | |
| | | | GCTGTGTGTCTGGGCGTGTTGTA | |
| | | | TGTCCTGCTGCCGATGCCTTCATTAAAATGATTTGGAA | |
| 209795_ | CD69 | tagtctaattgaatcccttaaact | >gi\|4502680\|ref\|NM_001781.1\|Homo sapiens CD69 | 30.48 |
| at | antigen | cagggagcatttataaatggcaaa | molecule (CD69), mRNA | |
| | (p60, early | tgcttatgaaac | AGACTCAACAAGAGCTCCAGCAAAGACTTTCACTGTAGCTTGACTTG | |
| | T-cell | taagatttgtaatatttctctctt | ACCTGAGATTAACTAGGGAATCT | |
| | activation | tttagagaaatttgccaatttact | TGAGAATAAAGATGAGCTCTGAAAATTGTTTCGTAGCAGAGAACAGC | |
| | antigen) | ttgttatttttc | TCTTTGCATCCGGAGAGTGGACA | |
| | | cccaaaaagaatgggatgatcgtg | AGAAAATGATGCCACCAGTCCCCATTTCTCAACACGTCATGAAGGGT | |
| | | tatttatttttttacttcctcagc | CCTTCCAAGTTCCTGTCCTGTGT | |
| | | tgtagacaggtc | GCTGTAATGAATGTGGTCTTCATCACCATTTTAATCATAGCTCTCAT | |
| | | cttttcgatggtacatatttcttt | TGCCTTATCAGTGGGCCAATACA | |
| | | gcctttataatcttttatacagtg | ATTGTCCAGGCCAATACACATTCTCAATGCCATCAGACAGCCATGTT | |
| | | tcttacagagaa | TCTTCATGCTCTGAGGACTGGGT | |
| | | aagacataagcaaagactatgagg | TGGCTACCAGAGGAAATGCTACTTTATTTCTACTGTGAAGAGGAGCT | |
| | | aatatttgcaagacatagaatagt | GGACTTCAGCCCAAAATGCTTGT | |
| | | gttggaaaatgt | TCTGAACATGGTGCTACTCTTGCTGTCATTGATTCTGAAAAGGACAT | |
| | | gcaatatgtgatgtggcaaatctc | GAACTTTCTAAAACGATACGCAG | |
| | | tattaggaaatattctgtaatctt | GTAGAGAGGAACACTGGGTTGGACTGAAAAAGGAACCTGGTCACCCA | |
| | | cagacatagaat | TGGAAGTGGTCAAATGGCAAAGA | |
| | | aatactagtcttataataggtttg | ATTTAACAACTGGTTCAACGTTACAGGGTCTGACAAGTGTGTTTTTC | |
| | | tgactttcctaaatcaattctatt | TGAAAAACACAGAGGTCAGCAGC | |
| | | acgtgcaatact | ATGGAATGTGAGAAGAATTTATACTGGATATGTAACAAACCTTACAA | |
| | | tcaatacttcat | ATAATAAGGAAACATGTTCACTT | |
| | | [Seq Id No 64] | ATTGACTATTATAGAATGGAACTCAAGGAAATCTGTGTCAGTGGATG | |
| | | | CTGCTCTGTGGTCCGAAGTCTTC | |
| | | | CATAGAGACTTTGTGAAAAAAAATTTTATAGTGTCTTGGGAATTTTC | |
| | | | TTCCAAACAGAACTATGGAAAAA | |
| | | | AAGGAAGAAATTCCAGGAAAATCTGCACTGTGGGCTTTTATTGCCAT | |
| | | | GAGCTAGAAGCATCACAGGTTGA | |
| | | | CCAATAACCATGCCCAAGAATGAGAAGAATGACTATGCAACCTTTGG | |
| | | | ATGCACTTTATATTATTTTGAAT | |
| | | | CCAGAAATAATGAAATAACTAGGCGTGGACTTACTATTTATTGCTGA | |
| | | | ATGACTACCAACAGTGAGAGCCC | |
| | | | TTCATGCATTTGCACTACTGGAAGGAGTTAGATGTTGGTACTAGATA | |
| | | | CTGAATGTAAACAAAGGAATTAT | |
| | | | GGCTGGTAACATAGGTTTTTAGTCTAATTGAATCCCTTAAACTCAGG | |
| | | | GAGCATTTATAAATGGACAAATG | |
| | | | CTTATGAAACTAAGATTTGTAATATTTCTCTCTTTTTAGAGAAATTT | |
| | | | GCCAATTTACTTTGTTATTTTTC | |
| | | | CCCAAAAAGAATGGGATGATCGTGTATTTATTTTTTTACTTCCTCAG | |
| | | | CTGTAGACAGGTCCTTTTCGATG | |
| | | | GTACATATTTCTTTGCCTTTATAATCTTTTATACAGTGTCTTACAGA | |
| | | | GAAAAGACATAAGCAAAGACTAT | |
| | | | GAGGAATATTTGCAAGACATAGAATAGTGTTGGAAAATGTGCAATAT | |
| | | | GTGATGTGGCAAATCTCTATTAG | |
| | | | GAAATATTCTGTAATCTTCAGACCTAGAATAATACTAGTCTTATAAT | |
| | | | AGGTTTGTGACTTTCCTAAATCA | |
| | | | ATTCTATTACGTGCAATACTTCAATACTTCATTTAAAATATTTTTAT | |
| | | | GTGCAATAAAATGTATTTGTTTG | |
| | | | TATTTTGTGTTCAGTACAATTATAAGCTGTTTTTATATATGTGAAAT | |
| | | | AAAAGTAGAATAAACACAAAAAA | |
| | | | AAAAAAAAAAAAAAAAAAAAAA | |
| 210038_ | protein | aatccattcatcctgattgggcat | >gi\|48255887\|ref\|NM_006257.2\|Homo sapiens | 19.42 |
| at | kinase C, | gaaatccatggtcaagaggacaag | protein kinase C, theta (PRKCQ), mRNA | |
| | theta | tggaaagtgagagggaaggtttgc | AGTCCCCGCGCAGTCCCCGCGCAcTCCCAGCGCCACCGGGCAGCAGC | |
| | | tagacaccttcgcttgttatcttg | GGCGCCGTGCTCGCTCCAGGGCG | |
| | | tcaagatagaaaagatagtatcat | CAACCATGTCGCCATTTCTTCGGATTGGCTTGTCCAACTTTGACTGC | |
| | | ttcacccttgccagtaaaaacctt | GCGTCCTGCCAGTCTTGTCAGGG | |
| | | tccatccacccattctcagcagac | CGAGGCTGTTAACCCTTACTGTGCTGTGCTCGTCAAAGAGTATGTCG | |
| | | tccagtattggc | AATCAGAGAACGGGCAGATGTAT | |
| | | acagtcactcactgccattctcac | ATCCAGAAAAAGCCTACCATGTACCCACCCTGGGACAGCACTTTTGA | |
| | | actataacaagaaaagaaatgaag | TGCCCATATCAACAAGGGAAGAG | |
| | | tgcataagtctc | TCATGCAGATCATTGTGAAAGGCAAAAACGTGGACCTCATCTCTGAA | |
| | | ctgggaaaagaaccttaacccctt | ACCACCGTGGAGCTCTACTCGCT | |
| | | ctcgtgccatgactggtgatttca | GGCTGAGAGGTGCAGGAAGAACAACGGGAAGACAGAAATATGGTTAG | |
| | | tgactcataagc | AGCTGAAACCTCAAGGCCGAATG | |
| | | ccctccgtaggcatcattcaagat | CTAATGAATGCAAGATACTTTCTGGAAATGAGTGACACAAAGGACAT | |
| | | caatggcccatgcatgctgtttgc | GAATGAATTTGAGACGGAAGGCT | |
| | | agca | TCTTTGCTTTGCATCAGCGCCGGGGTGCCATCAAGCAGGCAAAGGTC | |
| | | [Seq Id No 65] | CACCACGTCAAGTGCCACGAGTT | |
| | | | CACTGCCACCTTCTTCCCACAGCCCACATTTTGCTCTGTCTGCCACG | |
| | | | AGTTTGTCTGGGGCCTGAACAAA | |
| | | | CAGGGCTACCAGTGCCGACAATGCAATGCAGCAATTCACAAGAAGTG | |
| | | | TATTGATAAAGTTATAGCAAAGT | |
| | | | GCACAGGATCAGCTATCAATAGCCGAGAAACCATGTTCCACAAGGAG | |
| | | | AGATTCAAAATTGACATGCCACA | |
| | | | CAGATTTAAAGTCTACAATTACAAGAGCCCGACCTTCTGTGAACACT | |
| | | | GTGGGACCCTGCTGTGGGGACTG | |
| | | | GCACGGCAAGGACTCAAGTGTGATGCATGTGGCATGAATGTGCATCA | |
| | | | TAGATGCCAGACAAAGGTGGCCA | |
| | | | ACCTTTGTGGCATAAACCAGAAGCTAATGGCTGAAGCGCTGGCCATG | |
| | | | ATTGAGAGCACTCAACAGGCTCG | |
| | | | CTGCTTAAGAGATACTGAACAGATCTTCAGAGAAGGTCCGGTTGAAA | |
| | | | TTGGTCTCCCATGCTCCATCAAA | |
| | | | AATGAAGCAAGGCCGCCATGTTTACCGACACCGGGAAAAAGAGAGCC | |
| | | | TCAGGGCATTTCCTGGGAGTCTC | |
| | | | CGTTGGATGAGGTGGATAAAATGTGCCATCTTCCAGAACCTGAACTG | |
| | | | AACAAAGAAAGACCATCTCTGCA | |
| | | | GATTAAACTAAAAATTGAGGATTTTATCTTGCACAAAATGTTGGGGA | |
| | | | AAGGAAGTTTTGGCAAGGTCTTC | |
| | | | CTGGCAGAATTCAAGAAAACCAATCAATTTTTCGCAATAAAGGCCTT | |
| | | | AAAGAAAGATGTGGTCTTGATGG | |
| | | | ACGATGATGTTGAGTGCACGATGGTAGAGAAGAGAGTTCTTTCCTTG | |
| | | | GCCTGGGAGCATCCGTTTCTGAC | |
| | | | GCACATGTTTTGTACATTCCAGACCAAGGAAAACCTCTTTTTTGTGA | |
| | | | TGGAGTACCTCAACGGAGGGGAC | |
| | | | TTAATGTACCACATCCAAAGCTGCCACAAGTTCGACCTTTCCAGAGC | |
| | | | GACGTTTTATGCTGCTGAAATCA | |
| | | | TTCTTGGTCTGCAGTTCCTTCATTCCAAAGGAATAGTCTACAGGGAC | |
| | | | CTGAAGCTAGATAACATCCTGTT | |
| | | | AGACAAAGATGGACATATCAAGATCGCGGATTTTGGAATGTGCAAGG | |
| | | | AGAACATGTTAGGAGATGCCAAG | |
| | | | ACGAATACCTTCTGTGGGACACCTGACTACATCGCCCCAGAGATCTT | |
| | | | GCTGGGTCAGAAATACAACCACT | |
| | | | CTGTGGACTGGTGGTCCTTCGGGGTTCTCCTTTATGAAATGCTGATT | |
| | | | GGTCAGTCGCCTTTCCACGGGCA | |
| | | | GGATGAGGAGGAGCTCTTCCACTCCATCCGCATGGACAATCCCTTTT | |
| | | | ACCCACGGTGGCTGGAGAAGGAA | |
| | | | GCAAAGGACCTTCTGGTGAAGCTCTTCGTGCGAGAACCTGAGAAGAG | |
| | | | GCTGGGCGTGAGGGGAGACATCC | |
| | | | GCCAGCACCCTTTGTTTCGGGAGATCAACTGGGAGGAACTTGAACGG | |
| | | | AAGGAGATTGACCCACCGTTCCG | |
| | | | GCCGAAAGTGAAATCACCATTTGACTGCAGCAATTTCGACAAAGAAT | |
| | | | TCTTAAACGAGAAGCCCCGGCTG | |
| | | | TCATTTGCCGACAGAGCACTGATCAACAGCATGGACCAGAATATGTT | |
| | | | CAGGAACTTTTCCTTCATGAACC | |
| | | | CCGGGATGGAGCGGCTGATATCCTGAATCTTGCCCCTCCAGAGACAG | |
| | | | GAAAGAATTTGCCTTCTCCCTGG | |
| | | | GAACTGGTTCAAGAGACACTGCTTGGGTTCCTTTTTCAACTTGGAAA | |
| | | | AAGAAAGAAACACTCAACAATAA | |
| | | | AGACTGAGACCCGTTCGCCCCCATGTGACTTTTATCTGTAGCAGAAA | |
| | | | CCAAGTCTACTTCACTAATGACG | |
| | | | ATGCCGTGTGTCTCGTCTCCTGACATGTCTCACAGACGCTCCTGAAG | |
| | | | TTAGGTCATTACTAACCATAGTT | |
| | | | ATTTACTTGAAAGATGGGTCTCCGCACTTGGAAAGGTTTCAAGACTT | |
| | | | GATACTGCAATAAATTATGGCTC | |
| | | | TTCACCTGGGCGCCAACTGCTGATCAATGAAATGCTTGTTGAATCAG | |
| | | | GGGCAAACGGAGTACAGACGTCT | |
| | | | CAAGACTGAAACGGCCCCATTGCCTGGTCTAGTAGCGGATCTCACTC | |
| | | | AGCCGCAGACAAGTAATCACTAA | |
| | | | CCCGTTTTATTCTATTCCTATCTGTGGATGTGTAAATGGCTGGGGGG | |
| | | | CCAGCCCTGGATAGGTFTTTATG | |
| | | | GGAATTCTTTACAATAAACATAGCTTGTAACTTGAGATCTACAAATC | |
| | | | CATTCATCCTGATTGGGCATGAA | |
| | | | ATCCATGGTCAAGAGGACAAGTGGAAAGTGAGAGGGAAGGTTTGCTA | |
| | | | GACACCTTCGCTTGTTATCTTGT | |
| | | | CAAGATAGAAAAGATAGTATCATTTCACCCTTGCCAGTAAAAACCTT | |
| | | | TCCATCCACCCATTCTCAGCAGA | |
| | | | CTCCAGTATTGGCACAGTCACTCACTGCCATTCTCACACTATAACAA | |
| | | | GAAAAGAAATGAAGTGCATAAGT | |
| | | | CTCCTGGGAAAAGAACCTTAACCCCTTCTCGTGCCATGACTGGTGAT | |
| | | | TTCATGACTCATAAGCCCCTCCG | |
| | | | TAGGCATCATTCAAGATCAATGGCCCATGCATGCTGTTTGCAGCAGT | |
| | | | CAATTGAGTTGAATTAGAATTCC | |
| | | | AACCATACATTTTAAAGGTATTTGTGCTGTGTGTATATTTTGATAAA | |
| | | | ATGTTGTGACTTCATGGCAAACA | |
| | | | GGTGGATGTGTAAAAATGGAATAAAAAAAAAAAAAGAGTCAAAAAAA | |
| | | | AAAAAA | |
| 210915_ | T cell | Aaaggccacactggtgtgcctggc | >gi\|339011\|gb\|M15564.1\|HUMTCBYZ Human T-cell | 48.45 |
| x_at | receptor | cacaggtatcttccctgaccacgt | receptor rearranged beta-chain V-region (V-D-J) | |
| | beta | ggagctgagctggtgggtgaatgg | mRNA, complete cds | |
| | variable | gaaggaggtgcacagtggggtcag | GTAAAGCTCCCATCCTGCCCTGACCCTGCCATGGGCACCAGCCTCCT | |
| | 19///T | cacggacccgcagcccctcaagga | CTGCTGGATGGCCCTGTGTCTCC | |
| | cell | gcagcccgccctcaatgactccag | TGGGGGCAGATCACGCAGATACTGGAGTCTCCCAGAACCCCAGACAC | |
| | receptor | atactgcctgagcagccgcctgag | AACATCACAAAGAGGGGACAGAA | |
| | beta | ggtctcggccaccttctggcagaa | TGTAACTTTCAGGTGTGATCCAATTTCTGAACACAACCGCCTTTATT | |
| | constant 1 | cccccgcaaccacttccgctgtca | GGTACCGACAGACCCTGGGGCAG | |
| | | agtccagttctacgggctctcgga | GGCCCAGAGTTTCTGACTTACTTCCAGAATGAAGCTCAACTAGAAAA | |
| | | gaatgacgagtggacccaggatag | ATCAAGGCTGCTCAGTGATCGGT | |
| | | ggccaaacccgtcacccagatcgt | TCTCTGCAGAGAGGCCTAAGGGATCTTTCTCCACCTTGGAGATCCAG | |
| | | cagcgccgaggcctggggtagagc | CGCACAGAGCAGGGGGACTCGGC | |
| | | agactgtggctttacctcggtgtc | CATGTATCTCTGTGCCAGCAGCTTAGCAGGGTTGAATCAGCCCCAGC | |
| | | ctaccagcaaggggtcctgtctgc | ATTTTGGTGATGGGACTCGACTC | |
| | | caccatcctctatgagatcctgct | TCCATCCTAGAGGACCTGAACAAGGTGTTCCCACCCGAGGTCGCTGT | |
| | | agggaaggccaccatgtatgctgt | GTTTGAGCCATCAGAAGCAGAGA | |
| | | gctggtcagcgcccttgtgttgat | TCTCCCACACCCAAAAGGCCACACTGGTGTGCCTGGCCACAGGTATC | |
| | | ggccatggtcaagagaaaggattt | TTCCCTGACCACGTGGAGCTGAG | |
| | | ctgaaggcagccctggaagtggag | CTGGTGGGTGAATGGGAAGGAGGTGCACAGTGGGGTCAGCACGGACC | |
| | | ttaggagcttctaacccgtcatgg | CGCAGCCCCTCAAGGAGCAGCCC | |
| | | tttcaatacacattcttcttttgc | GCCCTCAATGACTCCAGATACTGCCTGAGCAGCCGCCTGAGGGTCTC | |
| | | cagc | GGCCACCTTCTGGCAGAACCCCC | |
| | | [Seq Id No 66] | GCAACCACTTCCGCTGTCAAGTCCAGTTCTACGGGCTCTCGGAGAAT | |
| | | | GACGAGTGGACCCAGGATAGGGC | |
| | | | CAAACCCGTCACCCAGATCGTCAGCGCCGAGGCCTGGGGTAGAGCAG | |
| | | | ACTGTGGCTTTACCTCGGTGTCC | |
| | | | TACCAGCAAGGGGTCCTGTCTGCCACCATCCTCTATGAGATCCTGCT | |
| | | | AGGGAAGGCCACCATGTATGCTG | |
| | | | TGCTGGTCAGCGCCCTTGTGTTGATGGCCATGGTCAAGAGAAAGGAT | |
| | | | TTCTGAAGGCAGCCCTGGAAGTG | |
| | | | GAGTTAGGAGCTTCTAACCCGTCATGGTTTCAATACACATTCTTCTT | |
| | | | TTGCCAGCGCTTCTGAAGAGCTG | |
| | | | CTCTCACCTCTCTGCATCCCAATA | |
| 210972_ | T cell | ggaacaagacttcaggtcacgctc | >gi\|338765\|gb\|M15565.1\|HUMTCAYE Human T-cell | 26.45 |
| x_at | receptor | gatatccagaaccctgaccctgcc | receptor rearranged alpha-chain V-region (V-D- | |
| | alpha | gtgtaccagctgagagactctaaa | J) mRNA, complete cds | |
| | locus/// | tccagtgacaagtctgtctgccta | TCCAAAATGAACGGTCTGTGGAAGGACATGAATAAAGCACAGGAGGT | |
| | T cell | ttcaccgattttgattctcaaaca | TGAAGTCAGATTTGCAGCTTTCT | |
| | receptor | aatgtgtcacaaagtaaggattct | AGGCAGGAGACAAGACAATCTGCATCITcACAGGAGGGATGGCCATG | |
| | delta | gatgtgtatatcacagacaaaact | CTCCTGGGGGCATcAGTGCTGAT | |
| | variable | gtgctagacatg | TCTGTGGCTTCAGCCAGACTGGGTAAACAGTCAACAGAAGAATGATG | |
| | 2///T cell | aggtctatggacttcaagagcaac | ACCAGCAAGTTAAGCAAAATTCA | |
| | receptor | agtgctgtggcctggagcaacaaa | CCATCCCTGAGCGTCCAGGAAGGAAGAATTTcTATTCTGAACTGTGA | |
| | alpha | tctgactttgcatgtgcaaacgcc | CTATACTAACAGCATGTTTGATT | |
| | variable | ttcaacaacagcattattccagaa | ATTTCCTATGGTACAAAAAATACCCTGCTGAAGGTCCTACATTCCTG | |
| | 20///T | gacaccttcttccccagcccagaa | ATATCTATAAGTTCCATTAAGGA | |
| | cell | agttcctgtgatgtcaagctggtc | TAAAAATGAAGATGGAAGATTCACTGTCTTCTTAAACAAAAGTGCCA | |
| | receptor | gagaaaagctttgaaacagatacg | AGCACCTCTCTCTGCACATTGTG | |
| | alpha | aacctaaacttt | CCCTCCCAGCCTGGAGACTCTGCAGTGTACTTCTGTGCAGCAAAGGG | |
| | joining | caaaacctgtcagtgattgggttc | GGCCGGCACTGCCAGTAAACTCA | |
| | 17///T | cgaatcctcctcctgaaagtggcc | CCTTTGGGACTGGAACAAGACTTCAGGTCACGCTCGATATCCAGAAC | |
| | cell | gggtttaatctg | CCTGACCCTGCCGTGTACCAGCT | |
| | receptor | ctcatgacgctgcggctgtggtcc | GAGAGACTCTAAATCCAGTGACAAGTCTGTCTGCCTATTCACCGATT | |
| | alpha | agctgagatctgcaagattgtaag | TTGATTCTCAAACAAATGTGTCA | |
| | constant | acagcctgtgct | CAAAGTAAGGATTCTGATGTGTATATCACAGACAAAACTGTGCTAGA | |
| | | ccct | CATGAGGTCTATGCACTTCAAGA | |
| | | [Seq Id No 67] | GCAACAGTGCTGTGGCCTGOAGCAACAAATCTGACTTTGCATGTGCA | |
| | | | AACGCCTTCAACAACAGCATTAT | |
| | | | TCCAGAAGACACCTTCTTCCCCAGCCCAGAAAGTTCCTGTGATGTCA | |
| | | | AGCTGGTCGAGAAAAGGTTTGAA | |
| | | | ACAGATACGAACCTAAACTTTCAAAACCTGTCAGTGATTGGGTTCCG | |
| | | | AATCCTCCTCCTGAAAGTGGCCG | |
| | | | GGTTTAATCTGCTCATGACGCTGCGGCTGTGGTCCAGCTGAGATCTG | |
| | | | CAAGATTGTAAGACAGCCTGTGC | |
| | | | TCCCTCGCTCCTTCCTCTGCATTGCCCCTCTTCTCCCTCTCcAJAACA | |
| | | | GAGGGAACTCTCCCACCCCCAAG | |
| | | | GAGGTGAAAGCTGCTACCACCCTCTGTGCC | |
| 211144_ | T cell | aaatgatacactactgctgcagct | >gi\|339406\|gb\|M30894.1\|HUMTCRGAD Human T-cell | 17.38 |
| x_at | receptor | cacaaacacctctgcatattacat | receptor Ti rearranged gamma-chain mRNA V-J-C | |
| | gamma | gtacctcctcct | region, complete cds | |
| | constant 2 | gctcctcaagagtgtggtctattt | GAATCAGGAAGACCAGCTCCTCCTACTGTCTTCTGTGTTACGGGATC | |
| | | tgccatcatcacctgctgtctgct | AGCGTTCCTTGTTGAGTGGGACC | |
| | | tggaagaacggc | TGAGTTTTGAGAGGGTCTTCTGCTCCTCTTGGTCTGGTCCCTTACTT | |
| | | tttctgctgcaatggagagaaatc | CCAAGAGCCCCAGAGAGGAAGGC | |
| | | ataacagacggtggcacaaggagg | ATGCTGTTGGCTCTAGCICTGCTTCTAGCTTTCCTGCCTCCTGCCAG | |
| | | ccatcttttcct | TCAGAAATCTTCCAACTTGGAAG | |
| | | catcggttattgtccctagaagcg | GGAGAACAAAGTCAGTCACCAGGCCAACTGGGTCATCAGCTGTAATC | |
| | | tcttctgaggatctagttgggctt | ACTTGTGATCTTCCTGTAGAAAA | |
| | | tctttctgggtt | TGCCGTCTACACCCACTGGTACCTACACCAGGAGGGGAAGGCCCCAC | |
| | | tgggccatttcagttctcatgtgt | AGCGTCTTCTGTACTATGACTCC | |
| | | gtactattctatcattattgtata | TACAACTCCAGGGTTGTGTTGGAATCAGGAATCAGTCGAGAAAAGTA | |
| | | atggttttcaaa | TCATACTTATGCAAGCACAGGGA | |
| | | ccagtgggcacacagagaacctca | AGAGCCTTAAATTTATACTGGAAAATCTAATTGAACGTGACTCTGGG | |
| | | gtctgtaataacaatgaggaatag | GTCTATTACTGTGCCACCTGGAA | |
| | | ccatggcgatctccagcaccaatc | GGATTATTATAAGAAAGTCTTTGGCAGTGGAACAACACTTGTTGTCA | |
| | | tctccatgttttccacagctcctc | CAGATAAACAACTTGATGCAGAT | |
| | | cagccaacccaaatagcgcctgct | GTTTCCCCCAAGCCCAcTATTTTTCTTCCTTCGATTGCTGAAACAAA | |
| | | atagtgtagacagcctgcggcttc | ACTCCAGAAGGCTGGAACATATC | |
| | | tagccttgtccctctcttagtgtt | TTTGTCTTCTTGAGAAATTTTTCCCAGATATTATTAAGATACATTGG | |
| | | ctttaatcagat | CAAGAAAAGAAGAGCAACACGAT | |
| | | aactgcctggaagcctttcatttt | TCTGGGATCCCAGGAGGGGAACACCATGAAGACTAACGACACATACA | |
| | | acacgccc | TGAAATTTAGCTGGTTAACGGTG | |
| | | [Seq Id No 68] | CCAGAAGAGTCACTGGACAAAGAACACAGATGTATCGTCAGACATGA | |
| | | | GAATAATAAAAACGGAATTGATC | |
| | | | AAGAAATTATCTTTCCTCCAATAAAGACAGATGTCACCACAGTGGAT | |
| | | | CCCAAAGACAGTTATTCAAAAGA | |
| | | | TGCAAATGATGTCACCACAGTGGATCCCAAATACAATTATTCAAAGG | |
| | | | ATGCAAATGATGTCATCACAATG | |
| | | | GATCCCAAAGACAATTGGTCAAAAGATGCAAATGATACACTACTGCT | |
| | | | GCAGCTCACAAACACCTCTGCAT | |
| | | | ATTACATGTACCTCCTCCTGCTCCTCAAGAGTGTGGTCTATTTTGCC | |
| | | | ATCATCACCTGCTGTCTGCTTGG | |
| | | | AAGAACGGCTTTCTGCTGCAATGGAGAGAAATCATAACAGACGGTGG | |
| | | | CACAAGGAGGCCATCTTTTCCTC | |
| | | | ATCGGTTATTGTCCCTAGAAGCGTCTTCTGAGGATCTAGTTGGGCTT | |
| | | | TCTTTCTGGGTTTGGGCCATTTC | |
| | | | AGTTCTCATGTGTGTACTATTCTATCATTATTGTATAATGGTTTTCA | |
| | | | AACCAGTGGGCACACAGAGAACC | |
| | | | TCAGTCTGTAATAACAATGAGGAATAGCCATGGCGATCTCCAGCACC | |
| | | | AATCTCTCCATGTTTTCCACAGC | |
| | | | TCCTCCAGCCAACCCAAATAGCGCCTGCTATAGTGTAGACAGCCTGC | |
| | | | GGCTTCTAGCCTTGTCCCTCTCT | |
| | | | TAGTGTTCTTTAATCAGATAACTGCCTGGAAGCCTTTCATTTTACAC | |
| | | | GCCCTGAAGCAGTCTTCTTTGCT | |
| | | | AGTTGAATTATGTGGTGTGTTTTTCCGTAATAAGCAAAATAAATTT | |
| 211796_ | T cell | gccatcagaagcagagatctccca | >gi\|3002924\|gb\|AF043179.1\|AF043179 Homo sapiens | 63.34 |
| s_at | receptor | cacccaaaaggccacactggtgtg | T cell receptor beta chain (TCRBV13S1-TCRBJ2S1) | |
| | beta | cctggccacaggtttctaccccga | mRNA, complete cds | |
| | variable | ccacgtggagctgagctggtgggt | ATGAGCATCGGCCTCCTGTGCTGTGCAGCCTTGTCTCTCCTGTGGGC | |
| | 21-1///T | gaatgggaaggaggtgcacagtgg | AGGTCCAGTGAATGCTGGTGTCA | |
| | cell | ggtcagcacagacccgcagcccct | CTCAGACCCCAAAATTCCAGGTCCTGAAGACAGGACAGAGCATGACA | |
| | receptor | caaggagcagcccgccctcaatga | CTGCAGTGTGCCCAGGATATGAA | |
| | beta | ctccagatactgcctgagcagccg | CCATGAATACATGTCCTGGTATCGACAAGACCCAGGCATGGGGCTGA | |
| | variable | cctgagggtctcggccaccttctg | GGCTGATTCATTACTCAGTTCGT | |
| | 19///T | gcagaacccccgcaaccacttccg | GCTGGTATCACTGACCAAGGAGAAGTCCCCAATGGCTACAATGTCTC | |
| | cell | ctgtcaagtccagttctacgggct | CAGATCAACCACAGAGGATTTCC | |
| | receptor | ctcggagaatgacgagtggaccca | CGCTCAGGCTGCTGTCGGCTGCTCCCTCCCAGACATCTGTGTACTTC | |
| | beta | ggatagggccaaacctgtcaccca | TGTGCCAGCAGTTTCCCCCGGCA | |
| | variable | gatcgtcagcgccgaggcctgggg | GCCGTCCTACAATGAGCAGTTCTTCGGGCCAGGGACACGGCTCACCG | |
| | 5-4///T | tagagcagactgtggcttcacctc | TGCTAGAGGACCTGAAAAACGTG | |
| | cell | cgagtcttaccagcaaggggtcct | TTCCCACCCGAGGTCGCTGTGTTTGAGCCATCAGAAGCAGAGATCTC | |
| | receptor | gtctgccaccatcctctatgagat | CCACACCCAAAAGGCCACACTGG | |
| | beta | cttgctagggaaggccaccttgta | TGTGCCTGGCCACAGGTTTCTACCCCGACCACGTGGAGCTGAGCTGG | |
| | variable | tgctgtgctggtcagtgccctcgt | TGGGTGAATGGGAAGGAGGTGCA | |
| | 3-1///T | gctgatggccatggtcaagagaaa | CAGTGGGGTCAGCACAGACCCGCAGCCCCTCAAGGAGCAGCCCGCCC | |
| | cell | gga | TCAATGACTCCAGATACTGCCTG | |
| | receptor | [Seq Id No 69] | AGCAGCCGCCTGAGGGTCTCGGCCACCTTCTGGCAGAACCCCCGCAA | |
| | beta | | CCACTTCCGCTGTCAAGTCCAGT | |
| | constant 1 | | TCTACGGGCTCTCGGAGAATGACGAGTGGACCCAGGATAGGGCCAAA | |
| | | | CCTGTCACCCAGATCGTCAGCGC | |
| | | | CGAGGCCTGGGGTAGAGCAGACTGTGGCTTCACCTCCGAGTCTTACC | |
| | | | AGCAAGGGGTCCTGTCTGCCACC | |
| | | | ATCCTCTATGAGATCTTGCTAGGGAAGGCCACCTTGTATGCTGTGCT | |
| | | | GGTCAGTGCCCTCGTGCTGATGG | |
| | | | CCATGGTCAAGAGAAAGGATTCCAGAGGCTGA | |
| 211902_ | T cell | gaatcgtttctctgtgaacttcca | >gi\|1100165\|gb\|L34703.1\|HUMTCRAZ Homo sapiens | 20.68 |
| x_at | receptor | gaaagcagccaaatccttcagtct | T-cell receptor alpha chain (TCRA) mRNA (HLA | |
| | alpha | caagatctcaga | A1, 24; B7, 8; DR 1, 3), complete cds | |
| | locus | ctcacagctgggggatgccgcgat | ATGGCATGCCCTGGCTTCCTGTGGGCACTTGTGATCTCCACCTGTCT | |
| | | gtatttctgtgcttataggagtgc | TGAATTTAGCATGGCTCAGACAG | |
| | | atactctggggc | TCACTCAGTCTCAACCAGAGATGTCTGTGCAGGAGGCAGAGACCGTG | |
| | | tgggagttaccaactcactttcgg | ACCCTGAGCTGCACATATGACAC | |
| | | gaaggggaccaaactctcggtcat | CAGTGAGAGTGATTATTATTTATTCTGGTACAAGCAGCCTCCCAGCA | |
| | | accaaatatccagaaccctgaccc | GGCAGATGATTCTCGTTATTCGC | |
| | | tgccgtgtaccagctgagagactc | CAAGAAGCTTATAAGCAACAGAATGCAACAGAGAATCGTTTCTCTGT | |
| | | taaatccagtgacaagtctgtctg | GAACTTCCAGAAAGCAGCCAAAT | |
| | | cctattcaccgattttgattctca | CCTTCAGTCTCAAGATCTCAGACTCACAGCTGGGGGATGCCGCGATG | |
| | | aacaaatgtgtcacaaagtaagga | TATTTCTGTGCTTATAGGAGTGC | |
| | | ttctgatgtgta | ATACTCTGGGGCTGGGAGTTACCAACTCACTTTCGGGAAGGGGACCA | |
| | | tatcacagacaaaactgtgctaga | AACTCTCGGTCATACCAAATATC | |
| | | catgaggtctatggacttcaagag | CAGAACCCTGACCCTGCCGTGTACCAGCTGAGAGACTCTAAATCCAG | |
| | | caacagtgctgt | TGACAAGTCTGTCTGCCTATTCA | |
| | | ggcctggagcaacaaatctgactt | CCGATTTTGATTCTCAAACAAATGTGTCACAAAGTAAGGATTCTGAT | |
| | | tgcatgtgcaaacgccttcaacaa | GTGTATATCACAGACAAAACTGT | |
| | | cagcattattcc | GCTAGACATGAGGTCTATGGACTTCAAGAGCAACAGTGCTGTGGCCT | |
| | | agaagacaccttcttccccagccc | GGAGCAACAAATCTGACTTTGCA | |
| | | agaaagttcctgtgatgtcaagct | TGTGCAAACGCCTTCAACAACAGCATTATTCCAGAAGACACCTTCTT | |
| | | ggtcgagaaaagctttgaaacaga | CCCCAGCCCAGAAAGTTCCTGTG | |
| | | tacgaacctaaactttcaaaacct | ATGTCAAGCTGGTCGAGAAAAGCTTTGAAACAGATACGAACCTAAAC | |
| | | gtcagtgattgggttccgaatcct | TTTCAAAACCTGTCAGTGATTGG | |
| | | cctcctgaaagtggccgggtttaa | GTTCCGAATCCTCCTCCTGAAAGTGGCCGGGTTTAATCTGCTCATGA | |
| | | tctgctcatgacgctgcggttgtg | CGCTGCGGTTGTGGTCCAGCTGA | |
| | | gtcc | | |
| | | [Seq Id No 70] | | |
| 212999_ | Major histo- | cactgcagaatgaaggaacatccc | >gi\|24797068\|ref\|NM_002123.2\|Homo sapiens | 15.79 |
| x_at | compatibility | ttgaggtgacccagccaacctgtg | major histocompatibility complex, class II, DQ | |
| | complex, | gccagaaggagqnttgtaccttga | beta 1 (HLA-DQB1), mRNA | |
| | class II, | aaagacactgaaagcattttggng | CAGATCCATCAGGTCCGAGCTGTGTTGACTACCACTTTTCCCTTCGT | |
| | DQ beta 1/// | tgtnaagtaagggtgggcagagga | CTCAATTATGTCTTGGAAAAAGG | |
| | Major histo- | ggtagaaaatcaattcaattgtcg | CTTTGCGGATCCCCGGAGGCCTTCGGGCAGCAACTGTGACCTTGATG | |
| | compatibility | catcattcatggttctttaatatt | CTGTCGATGCTGAGCACCCCAGT | |
| | complex, | gatgctcagtgcantggcctnaga | GGCTGAGGGCAGAGACTCTCCCGAGGATTTCGTGTACCAGTTTAAGG | |
| | class II, | atatcccagcctctcttctggttt | GCATGTGCTACTTCACCAACGGG | |
| | DQ beta 1 | gntgagtgctntntaagtaagcat | ACAGAGCGCGTGCGTCTTGTGAGCAGAAGCATCTATAACCGAGAAGA | |
| | | ggtngaattgtttggggncanata | GATCGTGCGCTTCGACAGCGACG | |
| | | tagtganccttggtcactggtgtt | TGGGGGAGTTCCGGGCGGTGACGCTGCTGGGGCTGCCTGCCGCCGAG | |
| | | tcaaacattctg | TACTGGAACAGCCAGAAGGACAT | |
| | | gnaagtcacatcnatcaagaatan | CCTGGAGAGGAAACGGGCGGCGGTGGACAGGGTGTGCAGACACAACT | |
| | | tttttanttttaagaaagcataac | ACCAGTTGGAGCTCCGCACGACC | |
| | | cagcaataaa | TTGCAGCGGCGAGTGGAGCCCACAGTGACCATCTCCCCATCCAGGAC | |
| | | [Seq Id No 71] | AGAGGCCCTCAACCACCACAACC | |
| | | | TGCTGGTCTGCTCGGTGACAGATTTCTATCCAGCCCAGATCAAAGTC | |
| | | | CGGTGGTTTCGGAATGACCAGGA | |
| | | | GGAGACAGCTGGCGTTGTGTCCACCCCCCTTATTAGGAATGGTGACT | |
| | | | GGACCTTCCAGATCCTGGTGATG | |
| | | | CTGGAAATGACTCCCCAGCGTGGAGACGTCTACACCTGCCACGTGGA | |
| | | | GCACCCCAGCCTCCAGAGCCCCA | |
| | | | TCACCGTGGAGTGGCGGGCTCAATCTGAATCTGCCCAGAGCAAGATG | |
| | | | CTGAGTGGCATTGGAGGCTTCGT | |
| | | | GCTGGGGCTGATCTTCCTCGGGCTGGGCCTTATCATCCATCACAGGA | |
| | | | GTCAGAAAGGGCTCCTGCACTGA | |
| | | | CTCCTGAGACTATTTTAACTGGGATTGGTTATCACTTTTCTGTAACG | |
| | | | CCTGCTTGTCCCTGCCCAGAATT | |
| | | | CCCAGCTGTCTGTGTCAGCCTGTCCCCCTGAGATCAGAGTCCTACAG | |
| | | | TGGCTGTCACGCAGCCACCAGGT | |
| | | | CATCTCCTTTCATCCCCACCTTGAGGCGGATGGCTGTGACCCTACTT | |
| | | | CCTGCACTGACCCACAGCCTCTG | |
| | | | CCTGTGCACGGCCAGCTGCATCTACTCAGGCCCCAAGGGGTTTCTGT | |
| | | | TTCTATTCTCTCCTCAGACTGCT | |
| | | | CAAGAGAAGCACATGAAAACCATTACCTGACTTTAGAGCTTTTTTAC | |
| | | | ATAATTAAACATGATCCTGAGTT | |
| 213193_ | T cell | Tgactccagatactgcctgagcag | >gi\|46184507\|gb\|AL559122.3\|AL559122 AL559122 | 44.75 |
| x_at | receptor | ccgcctgagggtctcggccacctt | Homo sapiens T CELLS (JURKAT CELL LINE) COT 10- | |
| | beta | ctggcagaacccccgcaaccactt | NORMALIZED Homo sapiens cDNA clone CS0DJ014YE01 | |
| | variable | ccgctgtcaagtccagttctacgg | 5-PRIME, mRNA sequence | |
| | 19///T | gctctcggagaatgacgagtggac | TGCCTGGCCACAGGCTTCTTCCCCGACCACGTGGAGCTGAGCTGGTG | |
| | cell | ccaggatagggccaaacccgtcac | GGTGAATGGGAAGGAGGTGCACA | |
| | receptor | ccagatcqtcagcgccgaggcctg | ATGGGGTCAACACAGACCCGCAGCCCCTCAAAGAACAGCCCGCCCTC | |
| | beta | gggtagagcagactgtggctttac | AATGACTCCAAATAMTGCCTGAG | |
| | variable | ctcggtgtcctaccagcaaggggt | CAGCCGCCTGAGGGTCTCGGCCACCTTCTGGCAGAACCCCCGCAACC | |
| | 19///T | cctgtctgccaccatcctctatga | ACTTCCGCTGTCAAGTCCAGTTC | |
| | cell | gatcctgctagggaaggccaccct | TACGGGCTCTCGGAGAATGACGAGTGGACCCAGGATAGGGCCAAACC | |
| | receptor | gtatgctgtgctggtcagcnccct | CGTCACCCAAATCGTCAGCGCCG | |
| | beta | tgtgttgatggccatggtcaagag | AGGCCTGGGGTARAGCARAMTGTGGCTTTACCTCGGTGTCCTACCAA | |
| | constant | aaaggatttctgaaggcagccctg | CAAGGGGTCCTGTCTGCCACCAT | |
| | 1///T | gaagtggagttaggagcttctaac | CCTCTATGARATCCTGcTAGGGAAGGCCACCcTGTATGCTGTGCTGG | |
| | cell | ccgtcatggtttcaatacacattc | TCAGCGCCCTTGTGTTGATGGCC | |
| | receptor | ttcttttgccagcgcttctgaaga | ATGGTCAAGAGAAAGGATTTCTGAAGGCAGCCCTGGAAGTGGAATTA | |
| | beta | gctgctctcacctctctgcatccc | AGAACTTCTAACCCGTCATGGTT | |
| | constant 1 | aatagatatccccctatgtgcatg | TCAATACACATTCTTCTTTTGCCAGCGCTTCTGAAGAGCTGCTCTCA | |
| | | cacacctgcacactcacggctgaa | CCTCTCTGCATCCCAATAGATAT | |
| | | atctccctaacccagggggacctt | CCCCCTATGTGCATGCACACCTGCACACTCACGGCTGAAATCTCCCT | |
| | | agcatgcctaagtga | AACCCAGGOGGACCTTACCATGC | |
| | | [Seq Id No 72] | CTAAGTGACTAAACCAATAAAAATGTTCTGGTCTGGCCTGA | |
| 213539_ | CD3D | gggaacactgctctcagacattac | >gi\|98985799\|ref\|NM_000732.4\|Homo sapiens CD3d | 34.66 |
| at | antigen, | aagactggacctgggaaaacgcat | molecule, delta (CD3-TCR complex) (CD3D), | |
| | delta poly- | cctggacccacgaggaatatatag | transcript variant 1, mRNA | |
| | peptide | gtgtaatgggacagatatatacaa | AGAGAAGCAGACATCTTCTAGTTCCTCCCCCACTCTCCTCTTTCCGG | |
| | (TiT3 | ggacaaagaatctaccgtgcaagt | TACCTGTGAGTCAGCTAGGGGAG | |
| | complex) | tcattatcgaatgtgccagagctg | GGCAGCTCTCACCCAGGCTGATAGTTCGGTGACCTGGCTTTATCTAC | |
| | | tgtggagctggatccagccaccgt | TGGATGAGTTCCGCTGGGAGATG | |
| | | ggctggcatcattgtcactgatgt | GAACATAGCACGTTTCTCTCTGCCCTGGTACTGGCTACCCTTCTCTC | |
| | | cattgccactctgctccttgcttt | GCAAGTGAGCCCCTTCAAGATAC | |
| | | gggagtcttctgctttgctggaca | CTATAGAGGAACTTGAGGACAGAGTGTTTGTGAATTGCAATACCAGC | |
| | | tgagactggaaggctgtctggggc | ATCACATGGGTAGAGGGAACGGT | |
| | | tgccgacacacaagctctgttgag | GGGAACACTGCTCTCAGACATTACAAGACTGGACCTGGGAAAACGCA | |
| | | gaatgaccaggtctatcagcccct | TCCTGGACCCACGAGGAATATAT | |
| | | ccgagatcgagatgatgctcagta | AGGTGTAATGGGACAGATATATACAAGGACAAAGAATCTACCGTGCA | |
| | | cagccaccttggaggaaactgggc | AGTTCATTATCGAATGTGCCAGA | |
| | | tcggaacaagtgaacctgagactg | GCTGTGTGAGCTGGATCCAGCCACCGTGGCTGGCATCATTGTCACT | |
| | | gtggcttctagaagcagccattac | GATGTCATTGCCACTCTGCTCCT | |
| | | caactgtacct | TGCTTTGGGAGTCTTCTGCTTTGCTGGACATGAGACTGGAAGGCTGT | |
| | | [Seq Id No 73] | CTGGGGCTGCCGACACACAAGCT | |
| | | | CTGTTGAGGAATGACCAGGTCTATCAGCCCCTCCGAGATCGAGATGA | |
| | | | TGCTCAGTACAGCCACCTTGGAG | |
| | | | GAAACTGGGCTCGGAACAAGTGAACCTGAGACTGGTGGCTTCTAGAA | |
| | | | GCAGCCATTACCAACTGTACCTT | |
| | | | CCCTTCTTGCTCAGCCAATAAATATATCCTCTTTCACTCAGAAAAAA | |
| | | | AAAAAAAAAAAAAAAAAAAAAAA | |
| | | | A | |
| 215806_ | T cell | aaatgatacactactgctgcagct | >gi\|339168\|gb\|M13231.1\|HUMTCGXH Human T-cell | 19.96 |
| x_at | receptor | cacaaacacctctgcatattacat | receptor aberrantly rearranged gamma-chain mRNA | |
| | gamma | gtacctcctcct | from cell line HPB-MLT | |
| | constant | gctcctcaagagtgtggtctattt | GGCATGCGGTGGGCCCTACTGGTGCTTCTAGCTTTCCTGTCTCCTGC | |
| | 2///T cell | tgccatcatcacctgctgtctgcn | CAGTCAGAAATCTTCCAACTTGG | |
| | receptor | tgnaagaacggc | AAGCGAGAACGAAGTCAGTCACCAGGCAGACTGGGTCATCTGCTGAA | |
| | gamma | nnnctgctgcaatggagagaantc | ATCACTTGCGATCTTACTGTAAC | |
| | variable | ataacagacggtggcacaaggagg | AAATACCTTCTACATCCACTGGTACCTACACCAGGAGGGGAAGGCCC | |
| | 9/// | ccnncntntcctcatcggnnattg | CACAGCGTCTTCTGTACTATGAC | |
| | similar to | tccctagaagcgtcttctgaggat | GTCTCCACTGCAAGGGATGTGTTGGAATCAGGACTCAGTCCAGGAAA | |
| | T-cell | ctagttgggctttctttctgggtt | GTATTATACTCATACACCCAGGA | |
| | receptor | tgggccatttcagttctcatgtgt | GGTGGAGCTGGATATTGAGACTGCAAAATCTAATTGAAAATGATTCT | |
| | gamma | gtactattctatcattattgtata | GGGGTCTATTACTGTGCCACCTG | |
| | chain C | atggttttcaaa | GGACAGGCAAAAATTATTATAAGAAACTCTTTGGCAGTGGAACAACA | |
| | region PT- | ccagtgggcacacagagaacctca | CTTGTTGTCACAGATAAACAACT | |
| | gamma- | gtctgtaataacaatgaggaatag | TGATGCAGATGTTTCCCCCAAGCCCACTATTTTTCTTCCTTCAATTG | |
| | ½/// | ccatggcgatctccagcaccaatc | CTGAAACAAAACTCCAGAAGGCT | |
| | similar to | tctccatgttttccacagctcctc | GGAACATACCTTTGTCTTCTTGAGAAATTTTTCCCAGATATTATTAA | |
| | T-cell | cagccaacccaaatagcgcctgct | GATACATTGGCAAGAAAAGAAGA | |
| | receptor | atagtgtaganannctgcggcttc | GCAACACGATTCTGGGATCCCAGGAGGGGAACACCATGAAGACTAAC | |
| | gamma | tagccttgtccctctcttagtgtt | GACACATACATGAAATTTAGCTG | |
| | chain V | ctttaatcagat | GTTAACGGTGCCAGAAGAGTCACTGGACAAAGAACACAGATGTATCG | |
| | region PT- | aactgcctggaagcctttcatttt | TCAGACATGAGAATAATAAAAAC | |
| | gamma- | acacgccctgaagcagtcttcttt | GGAATTGATCAAGAAATTATCTTTCCTCCAATAAAGACAGATGTCAC | |
| | ½ pre- | gcta | CACAGTGGATCCCAAAGACAGTT | |
| | cursor/// | [Seq Id No 74] | ATTCAAAAGATGCAAATGATGTCATCACAATGGATCCCAAAGACAAT | |
| | TCR gamma | | TGGTCAAAAGATGCAAATGATAC | |
| | alternate | | ACTACTGCTGCAGCTCACAAACACCTCTGCATATTACATGTACCTCC | |
| | reading | | TCCTGCTCCTCAAGAGTGTGGTC | |
| | frame | | TATTTTGCCATCATCACCTGCTGTCTGCTTGGAAGAACGGCTTTCTG | |
| | protein | | CTGCAATGGAGAGAAATCATAAC | |
| | | | AGACGGTGGCACAAGGAGGCCATCTTTTCCTCATCGGTTATTGTCCC | |
| | | | TAGTAGAAGCGTCTTCTGAGGATCTA | |
| | | | GTTGGGCTTTCTTTCTGGGTTTGGGCCATTTCAGTTCTCATGTGTGT | |
| | | | ACTATTCTATCATTATTGTATAA | |
| | | | TGGTTTTCAAACCAGTGGGCACACAGAGAACCTCAGTCTGTAATAAC | |
| | | | AATGAGGAATAGCCATGCCGATC | |
| | | | TCCAGCACCAATCTCTCCATGTTTTCCACAGCTCCTCCAGCCAACCC | |
| | | | AAATAGCGCCTGCTATAGTGTAG | |
| | | | ACAGCCTGCGGCTTCTAGCCTTGTCCCTCTCTTAGTGTTCTTTAATC | |
| | | | AGATAACTGCCTGGAAGCCTTTC | |
| | | | ATTTTACACGCCCTGAAGCAGTCTTCTTTGCTAGTTGAATTATGTGG | |
| | | | TGTGTTTTTCCGTAATAAGCAAA | |
| | | | ATAAATTTAAAAAAATGAAAA | |
| 216748_ | pyrin and | ccaccctctggatcccaatattga | >gi\|10437303\|dbj\|AK024890.1\|Homo sapiens cDNA: | 10.74 |
| at | HIN domain | gatcttatcctcagggaatcctca | FLJ21237 fis, clone COL01114 | |
| | family | cttagacccctg | GTCAAAGGAATAATCCCATCTAAAAAGACGAAACAGAAAGAAGTGTA | |
| | member 1 | taacaggttaaatcttcatggtgt | TCCTGCTACACCTGCATGCACCC | |
| | | tctgtttcctaggaacttctttct | CAAGCAACCGTCTCACAGCTAAAGGAGCAGAGGAGACTCTTGGACCT | |
| | | tttctactgttt | CAGGTAAGCTTCAGGAAGAGGAG | |
| | | atgacaactgaagttaataagtgt | CAGGCTTCAAGTCTCACAGTGGAAGCTCTGCTGTGGCTGTTCCACTC | |
| | | ttatctttcccacctactcaaagt | AATCTGTCCAGCAGGCAGTTATT | |
| | | agttccaagatt | TCTTCATATGTTTCCCATCAAGTTTCAGATTTATCAAATTACATAAT | |
| | | agggctagtttgtaattctgtgga | AATTGATCATCTTTCTGCAAGGC | |
| | | ccactgtaaacgagggcctagttc | AACAAGTTAAACGCTTTAGTAAACATAATGTAAATATACATAAAATA | |
| | | agtgtctgcctc | AATATAATATTTTCATCTCCAAT | |
| | | atgggaagcttccaataaatacct | AGAGAAGGATGTTAACTTGAGAGTCAGATAAAAAAACGTTTGCCTAT | |
| | | ttg | GTTTACAAAAGCCTAGTTTCTTA | |
| | | [Seq Id No 75] | ACTGCAAGTCAGCATATCCCAAAACACAAGTAATTAAGGAATGATGT | |
| | | | GTGTTACTTTCTCTGCTCCCTTT | |
| | | | TTAAAAATGAAACCATCTATGCCATGTTCTTTCAATTGGCCTGGGGA | |
| | | | TGTACTTAAGTTTCCAAGAAAAA | |
| | | | CAATTTATATACAATAAATATATTACCTTGTAATGAAAATGTGCTCT | |
| | | | GCTTCATTTGACACTGAAAGTAA | |
| | | | TTAACAAGAAAAATAAACTACTTGTAGAAAAGAAAAAAACCATCTGA | |
| | | | AGAAGAGACTGGAACCAAAAGGA | |
| | | | GTAAGATGTCCAAAGAGCAGACTCGGCCTTCCTGCTCTGCAGGAGCC | |
| | | | AGCACGTCCACAGCCATGGGCCG | |
| | | | TTCCCCACCTCCCCAGACCTCATCATCAGCTCCACCCAACACTTCCT | |
| | | | CAACTGAGGTACACTCTTCCTGG | |
| | | | TCCCCTTTTGATTCATTTTCTTCAACCCAAAATGTAGGAATCTGATT | |
| | | | TCATCTTCTACTGAAAAATGACA | |
| | | | TCAATCATCAGCCAGTAAATCAAATGTATAGACTGAGAATTAACTGC | |
| | | | ATTTTAATCTTTTGCTTCCACAG | |
| | | | GCATATTTGATGAACTTGACATTATCTCTGACTGCAGGAAGTTTTCT | |
| | | | GTCCTGTGCTGTTTGGGGAAGAG | |
| | | | ACAGAGAACTGCGGAATCTGGAACTTTCAGCAACAGACTCACTGTCT | |
| | | | ACTGCCCCCATCTATTATACACC | |
| | | | CATTCCCTTTGCTCACTAATTTGTTCAAGTTTCTCTGACATACACCA | |
| | | | TGCTCCTTTTTCCTTTAGGATTT | |
| | | | TCACACACCATATTTCTTTCACCTTTAAACTCTTACCTGGCCAACCC | |
| | | | TATCCACCCTCTGGATCCCAATA | |
| | | | TTGAGATCTTATCCTCAGGGAATCCTCACTTAGACCCCTGTAACAGG | |
| | | | TTAAATCTTCATGGTGTTCTGTT | |
| | | | TCCTAGGAACTTCTTTCTTTTCTACTGTTTATGACAACTGAAGTTAA | |
| | | | TAAGTGTTTATCTTTCCCACCTA | |
| | | | CTCAAAGTAGTTCCAAGATTAGGGCTAGTTTGTAATTCTGTGGACCA | |
| | | | CTGTAAACGAGGGCCTAGTTCAG | |
| | | | TGTCTGCCTCATGGGAAGCTTCCAATAAATACCTTTGCTCAACGAAA | |
| | | | AAATGAAAACCCAGTGGCTCACG | |
| | | | CCTGTAATCCCAGCACTTTGGGAGGCCGAGGCAGGTGGATTGCCTGA | |
| | | | GGTCAGGAGTTTGAGACTAGTCT | |
| | | | GGCCAACATGGTGAAACACTATCTCTACTAAAAATACAAAAAAATTA | |
| | | | GCTGGGTATGGTGGCTTACGCCT | |
| | | | ATAATCCCAGCTACTCAGGAGGCTGAGGCAGGGGAATTGCTTGAACC | |
| | | | AGGGAGGTGGAGGTTGCAGTGAG | |
| | | | CTGAGATCGCACCACTGCACTCCAGCCTGGGTGACAGAGCGAGACTC | |
| | | | CATCTCAAAATAAAAAAGTAAAA | |
| | | | AAAAAAAAAAAAAA | |
| 217147_ | T cell | tctcctttctcaccaatgggcaat | >gi\|54607136\|ref\|NM_016388.2\|Homo sapiens T | 24.16 |
| s_at | receptor | agcccataattgaaataaatttct | cell receptor associated transmembrane adaptor | |
| | associated | gattgaaaggta | 1 (TRAT1), mRNA | |
| | transmembrane | taggaaacattaaaatgcattact | GAGGCACAGATAAAGATAAGTTTTACTGTCATGCTGCTTTTAACATA | |
| | adaptor 1 | aagagaagtaatataattttctta | ACAGAGCAACATCACCTAGGAAA | |
| | | caaagtattttt | AAAGTTTGTAGGAGGATTTTTAATCCATATATTTGTCTTATGGCTAG | |
| | | cccaaagatagctttactatttca | ATAAAGATTTCTCTGAAAAAAAG | |
| | | aaaattgtcaaattaatgcatgct | AAGCATGTCAGGAATCTCTGGGTGCCCCTTTTTCCTCTGGGGACTTC | |
| | | ccttacaacaaa | TAGCATTGTTGGGCTTGGCTTTG | |
| | | caaatatcaaaaagagtttaggaa | GTTATATCACTGATCTTCAATATTTCCCACTATGTGGAAAAGCAACG | |
| | | ttctactagccagagatagtcact | ACAAGATAAAATGTACAGCTACT | |
| | | tggagaaacttt | CCAGTGACCACACCAGGGTTGATGAGTATTATATTGAAGACACACCA | |
| | | ctatatatccttctaaatattttt | ATTTATGGTAACTTAGATGATAT | |
| | | ctgggcatgctcatgtatgtacat | GATTTCAGAACCAATGGATGAAAATTGCTATGAACAAATGAAAGCCC | |
| | | cagttgtttctt | GACCAGAGAAATCTGTAAATAAG | |
| | | tttattttgaaccaaaaatgtggt | ATGCAGGAAGCCACCCCATCTGCACACGCAACCAATGAAACACAGAT | |
| | | ttcttttgtacacattacttaaac | GTGCTACGCCTCACTTGATCACA | |
| | | tttctttccagt | GCGTTAACGGGAAGCGTAGAAAGCCCAGGAAACAGAATACTCATTTC | |
| | | caacaatatattgtggatttattt | TCAGACAAGGATGGAGATGAGCA | |
| | | tcactgttatatttaactatatat | ACTACATGCAATAGATGCCAGCGTTTCTAAGACCACCTTAGTAGACA | |
| | | aaatacgcatat | GTTTCTCCCCAGAAAGCCAGGCA | |
| | | attgtaattttaatgtctgcttag | GTAGAGGAAAACATTCATGATGATCCCATCAGACTGTTTGGATTGAT | |
| | | caccccactgataaccaaatcaca | CCGTGCTAAGAGAGAACCTATAA | |
| | | g | ACTAGCTGGACCATGATCTAGTTCAATGATTTGGCTCCTATTGAAGA | |
| | | [Seq Id No 76] | TGGCTTCTAAGAAAACAAGATGC | |
| | | | ACAGAGGACACAGAAGGACTTGGCAGCAGGGTGATGACCTGATCATT | |
| | | | TGTTGATGGGATGGTGGCTTACC | |
| | | | TCTTATTCACAGCTTACACTTATGCATGCCAAATGTAAGGCCATGAA | |
| | | | AATCAGTATTTCAAATAACTTAA | |
| | | | AAAATGCTTTACTACTAAAATGTAAAAAATTAATGTGCTCACCTCGG | |
| | | | CAGCACATATACTAAAAATTAAT | |
| | | | AAGACCCAGCTTGAAAATTGAGCCTGATAACAAGATTACAAATTCAC | |
| | | | AATACCTAATACTTAGGCAAATA | |
| | | | TAAAAATTTAAGCATGAATGCGTTCTGGAACACGTTAGAAGAAAAAT | |
| | | | AAAAGCCAATGAGTTTTTTTTTA | |
| | | | ATTCTCCTTTCTCACCAATGGGCAATAGCCCATAATTGAAATAAATT | |
| | | | TCTGATTGAAAGGTATAGGAAAC | |
| | | | ATTAAAATGCATTACTAAGAGAAGTAATATAATTTTCTTACAAAGTA | |
| | | | TTTTTCCCAAAGATAGCTTTACT | |
| | | | ATTTCAAAAATTGTCAAATTAATGCATGCTCCTTACAACAAACAAAT | |
| | | | ATCAAAAAGAGTTTAGGAATTCT | |
| | | | ACTAGCCAGAGATAGTCACTTGGAGAAACTTTCTATATATCCTTCTA | |
| | | | AATATTTTTCTGGGCATGCTTAT | |
| | | | GTATGTACATCAGTTGTTTCTTTTTATTTTGAACCAAAAATGTGGTT | |
| | | | TCTTTTGTACACATTACTTAAAC | |
| | | | TTTCTTTCCAGTCAACAATATATTGTGGATTTATTTTCACTGTTATA | |
| | | | TTTAACTATATATAAATACGCAT | |
| | | | ATATTGTAATTTTAATGTCTGCTTAGCACCCCACTGATAACCAAATC | |
| | | | ACAGTTTATTTAAATAATTTTAA | |
| | | | TGACTTTTCAAAAACAATTTATTGATGCAAAAAGCAAGGTTGAGATG | |
| | | | ACAATGTTTCTTTCAATAATTAA | |
| | | | AAAATACTGCTTCAC | |
| 219159_ | SLAM | Gagagtggacatttgtcgggaaac | >gi\|19923571\|ref\|NM_021181.3\|Homo sapiens SLAM | 15.84 |
| s_at | family | tcctaacatatgcccccattctgg | family member 7 (SLAMF7), mRNA | |
| | member 7 | agagaacacagagtacgacacaat | CTTCCAGAGAGCAATATGGCTGGTTCCCCAACATGCCTCACCCTCAT | |
| | | ccctcacactaatagaacaatcct | CTATATCCTTTGGCAGCTCACAG | |
| | | a | GGTCAGCAGCCTCTGGACCCGTGAAAGAGCTGGTCGGTTCCGTTGGT | |
| | | [Seq Id No 77] | GGGGCCGTGACTTTCCCCCTGAA | |
| | | | GTCCAAAGTAAAGCAAGTTGACTCTATTGTCTGGACCTTCAACACAA | |
| | | | CCCCTCTTGTCACCATACAGCCA | |
| | | | GAAGGGGGCACTATCATAGTGACCCAAAATCGTAATAGGGAGAGAGT | |
| | | | AGACTTCCCAGATGGAGGCTACT | |
| | | | CCCTGAAGCTCAGCAAACTGAAGAAGAATGACTCAGGGATCTACTAT | |
| | | | GTGGGGATATACAGCTCATCACT | |
| | | | CCAGCAGCCCTCCACCCAGGAGTACGTGCTGCATGTCTACGAGCACC | |
| | | | TGTCAAAGCCTAAAGTCACCATG | |
| | | | GGTCTGCAGAGCAATAAGAATGGCACCTGTGTGACCAATCTGACATG | |
| | | | CTGCATGGAACATGGGGAAGAGG | |
| | | | ATGTGATTTATACCTGGAAGGCCCTGGGGCAAGCAGCCAATGAGTCC | |
| | | | CATAATGGGTCCATCCTCCCCAT | |
| | | | CTCCTGGAGATGGGGAGAAAGTGATATGACCTTCATCTGCGTTGCCA | |
| | | | GCAACCCTGTCAGCAGAAACTTC | |
| | | | TCAAGCCCCATCCTTGCCAGGAAGCTCTGTGAAGGTGCTGCTGATGA | |
| | | | CCCAGATTCCTCCATGGTCCTCC | |
| | | | TGTGTCTCCTGTTGGTGCCCCTCCTGCTCAGTCTCTTTGTACTGGGG | |
| | | | CTATTTCTTTGGTTTCTGAAGAG | |
| | | | AGAGAGACAAGAAGAGTACATTGAAGAGAAGAAGAGAGTGGACATTT | |
| | | | GTCGGGAAACTCCTAACATATGC | |
| | | | CCCCATTCTGGAGAGAACACAGAGTACGACACAATCCCTCACACTAA | |
| | | | TAGAACAATCCTAAAGGAAGATC | |
| | | | CAGCAAATACGGTTTACTCCACTGTGGAAATACCGAAAAAGATGGAA | |
| | | | AATCCCCACTCACTGCTCACGAT | |
| | | | GCCAGACACACCAAGGCTATTTGCCTATGAGAATGTTATCTAGACAG | |
| | | | CAGTGCACTCCCCTAAGTCTCTG | |
| | | | CTCAAAAAAAAAACAATTCTCGGCCCAAAGAAAACAATCAGAAGAAT | |
| | | | TCACTGATTTGACTAGAAACATC | |
| | | | AAGGAAGAATGAAGAACGTTGACTTTTTTCCAGGATAAATTATCTCT | |
| | | | GATGCTTCTTTAGATTTAAGAGT | |
| | | | TCATAATTCCATCCACTGCTGAGAAATCTCCTCAAACCCAGAAGGTT | |
| | | | TAATCACTTCATCCCAAAAATGG | |
| | | | GATTGTGAATGTCAGCAAACCATAAAAAAAGTGCTTAGAAGTATTCC | |
| | | | TATAGAAATGTAAATGCAAGGTC | |
| | | | ACACATATTAATGACAGCCTGTTGTATTAATGATGGCTCCAGGTCAG | |
| | | | TGTCTGGAGTTTCATTCCATCCC | |
| | | | AGGGCTTGGATGTAAGGATTATACCAAGAGTCTTGCTACCAGGAGGG | |
| | | | CAAGAAGACCAAAACAGACAGAC | |
| | | | AAGTCCAGCAGAAGCAGATGCACCTGACAAAAATGGATGTATTAATT | |
| | | | GGCTCTATAAACTATGTGCCCAG | |
| | | | CACTATGCTGAGCTTACACTAATTGGTCAGACGTGCTGTCTGCCCTC | |
| | | | ATGAAATTGGCTCCAAATGAATG | |
| | | | AACTACTTTCATGAGCAGTTGTAGCAGGCCTGACCACAGATTCCCAG | |
| | | | AGGGCCAGGTGTGGATCCACAGG | |
| | | | ACTTGAAGGTCAAAGTTCACAAAGATGAAGAATCAGGGTAGCTGACC | |
| | | | ATGTTTGGCAGATACTATAATGG | |
| | | | AGACACAGAAGTGTGCATGGCCCAAGGACAAGGACCTCCAGCCAGGC | |
| | | | TTCATTTATGCACTTGTGCTGCA | |
| | | | AAAGAAAAGTCTAGGTTTTAAGGCTGTGCCAGAACCCATCCCAATAA | |
| | | | AGAGACCGAGTCTGAAGTCACAT | |
| | | | TGTAAATCTAGTGTAGGAGACTTGGAGTCAGGCAGTGAGACTGGTGG | |
| | | | GGCACGGGGGGCAGTGGGTACTT | |
| | | | GTAAACCTTTAAAGATGGTTAATTCATTCAATAGATATTTATTAAGA | |
| | | | ACCTATGCGGCCCGGCATGGTGG | |
| | | | CTCACACCTGTAATCCCAGCACTTTGGGAGGCCAAGGTGGGTGGGTC | |
| | | | ATCTGAGGTCAGGAGTTCAAGAC | |
| | | | CAGCCTGGCCAACATGGTGAAACCCCATCTCTACTAAAGATACAAAA | |
| | | | ATTTGCTGAGCGTGGTGGTGTGC | |
| | | | ACCTGTAATCCCAGCTACTCGAGAGGCCAAGGCATGAGAATCGCTTG | |
| | | | AACCTGGGAGGTGGAGGTTGCAG | |
| | | | TGAGCTGAGATGGCACCACTGCACTCCGGCCTAGGCAACGAGAGCAA | |
| | | | AACTCCAATACAAACAAACAAAC | |
| | | | AAACACCTGTGCTAGGTCAGTCTGGCACGTAAGATGAACATCCCTAC | |
| | | | CAACACAGAGCTCACCATCTCTT | |
| | | | ATACTTAAGTGAAAAACATGGGGAAGGGGAAAGGGGAATGGCTGCTT | |
| | | | TTGATATGTTCCCTGACACATAT | |
| | | | CTTGAATGGAGACCTCCCTACCAAGTGATGAAAGTGTTGAAAAACTT | |
| | | | AATAACAAATGCTTGTTGGGCAA | |
| | | | GAATGGGATTGAGGATTATCTTCTCTCAGAAAGGCATTGTGAAGGAA | |
| | | | TTGAGCCAGATCTCTCTCCCTAC | |
| | | | TGCAAAACCCTATTGTAGTAAAAAAGTCTTCTTTACTATCTTAATAA | |
| | | | AACAGATATTGTGAGATTCAAAA | |
| | | | AAAAAAAAAAAA | |
| 222838_ | SLAM | aacacctgtgctaggtcagtctgg | >gi\|19923571\|ref\|NM_021181.3\|Homo sapiens SLAM | 22.54 |
| at | family | cacgtaagatgaacatccctacca | family member 7 (SLAMF7), mRNA | |
| | member 7 | acacagagctcaccatctcttata | CTTCCAGAGAGCAATATGGCTGGTTCCCCAACATGCCTCACCCTCAT | |
| | | cttaagtgaaaaacatggggaagg | CTATATCCTTTGGCAGCTCACAG | |
| | | ggaaaggggaatggctgcttttga | GGTCAGCAGCCTCTGGACCCGTGAAAGAGCTGGTCGGTTCCGTTGGT | |
| | | tatgttccctgacacatatcttga | GGGGCCGTGACTTTCCCCCTGAA | |
| | | atggagacctccctaccaagtgat | GTCCAAAGTAAAGCAAGTTGACTCTATTGTCTGGACCTTCAACACAA | |
| | | gaaagtgttgaa | CCCCTCTTGTCACCATACAGCCA | |
| | | aaacttaataacaaatgcttgttg | GAAGGGGGCACTATCATAGTGACCCAAAATCGTAATAGGGAGAGAGT | |
| | | ggcaagaatgggattgaggattat | AGACTTCCCAGATGGAGGCTACT | |
| | | cttctctcagaa | CCCTGAAGCTCAGCAAACTGAAGAAGAATGACTCAGGGATCTACTAT | |
| | | aggcattgtgaagaattgagcca | GTGGGGATATACAGCTCATCACT | |
| | | gatctctctccctactgcaaaacc | CCAGCAGCCCTCCACCCAGGAGTACGTGCTGCATGTCTACGAGCACC | |
| | | ctattgtagta | TGTCAAAGCCTAAAGTCACCATG | |
| | | [Seq Id No 78] | GGTCTGCAGAGCAATAAGAATGGCACCTGTGTGACCAATCTGACATG | |
| | | | CTGCATGGAACATGGGGAAGAGG | |
| | | | ATGTGATTTATACCTGGAAGGCCCTGGGGCAAGCAGCCAATGAGTCC | |
| | | | CATAATGGGTCCATCCTCCCCAT | |
| | | | CTCCTGGAGATGGGGAGAAAGTCATATGACCTTCATCTGCGTTGCCA | |
| | | | GGAACCCTGTCAGCAGAAACTTC | |
| | | | TCAAGCCCCATCCTTGCCAGGAAGCTCTGTGAAGGTGCTGCTGATGA | |
| | | | CCCAGATTCCTCCATGGTCCTCC | |
| | | | TGTGTCTCCTGTTGGTGCCCCTCCTGCTCAGTCTCTTTGTACTGGGG | |
| | | | CTATTTCTTTGGTTTCTGAAGAG | |
| | | | AGAGACACAAGAAGAGTACATTGAAGAGAAGAAGAGAGTGGACATTT | |
| | | | GTCGGGAAACTCCTAACATATGC | |
| | | | CCCCATTCTGGAGAGAACACAGAGTACGACACAATCCCTCACACTAA | |
| | | | TAGAACAATCCTAAAGGAAGATC | |
| | | | CAGCAAATACGGTTTACTCCACTGTGGAAATACCGAAAAAGATGGAA | |
| | | | AATCCCCACTCACTGCTCACGAT | |
| | | | GCCAGACACACCAAGGCTATTTGCCTATGAGAATGTTATCTAGACAG | |
| | | | CAGTGCACTCCCCTAAGTCTCTG | |
| | | | CTCAAAAAAAAAACAATTCTCGGCCCAAAGAAAACAATCAGAAGAAT | |
| | | | TCACTGATTTGACTAGAAACATC | |
| | | | AAGGAAGAATGAAGAACGTTGACTTTTTTCCAGGATAAATTATCTCT | |
| | | | GATGCTTCTTTAGATTTAAGAGT | |
| | | | TCATAATTCCATCCACTGCTGAGAAATCTCCTCAAACCCAGAAGGTT | |
| | | | TAATCACTTCATCCCAAAAATGG | |
| | | | GATTGTGAATGTCAGCAAACCATAAAAAAAGTGCTTAGAAGTATTCC | |
| | | | TATAGAAATGTAAATGCAAGGTC | |
| | | | ACACATATTAATGACAGCCTGTTGTATTAATGATGGCTCCAGGTCAG | |
| | | | TGTCTGGAGTTTCATTCCATCCC | |
| | | | AGGGCTTGGATGTAAGGATTATACCAAGAGTCTTGCTACCAGGAGGG | |
| | | | CAAGAAGACCAAAACAGACAGAC | |
| | | | AAGTCCAGCAGAAGCAGATGCACCTGACAAAAATGGATGTATTAATT | |
| | | | GGCTCTATAAACTATGTGCCCAG | |
| | | | CACTATGCTGAGCTTACACTAATTGGTCAGACGTGCTGTCTGCCCTC | |
| | | | ATGAAATTGGCTCCAAATGAATG | |
| | | | AACTACTTTCATGAGCAGTTGTAGCAGGCCTGACCACAGATTCCCAG | |
| | | | AGGGCCAGGTGTGGATCCACAGG | |
| | | | ACTTGAGGTCAAAGTTCACAAAGATGAAGAATCAGGGTAGCTGACC | |
| | | | ATGTTTGGCAGATACTATAATGG | |
| | | | AGACACAGAAGTGTGCATGGCCCAAGGACAAGGACCTCCAGCCAGGC | |
| | | | TTCATTTATGCACTTGTGCTGCA | |
| | | | AAAGAAAAGTCTAGGTTTTAAGGCTGTGCCAGAACCCATCCCAATAA | |
| | | | AGAGACCGAGTCTGAAGTCACAT | |
| | | | TGTAAATCTAGTGTAGGAGACTTGGAGTCAGGCAGTGAGACTGGTGG | |
| | | | GGCACGGGGGGCAGTGGGTACTT | |
| | | | GTAAACCTTTAAAGATGGTTAATTCATTCAATAGATATTTATTAAGA | |
| | | | ACCTATGCGGCCCGGCATGGTGG | |
| | | | CTCACACCTGTAATCCCAGCACTTTGGGAGGCCAAGGTGGGTGGGTC | |
| | | | ATCTGAGGTCAGGAGTTCAAGAC | |
| | | | CAGCCTGGCCAACATGGTGAAACCCCATCTCTACTAAAGATACAAAA | |
| | | | ATTTGCTGAGCGTCGTGGTGTGC | |
| | | | ACCTGTAATCCCAGCTACTCGAGAGGCCAAGGCATGAGAATCGCTTG | |
| | | | AACCTGGGAGGTGGAGGTTGCAG | |
| | | | TGAGCTGAGATGGCACCACTGCACTCCGGCCTAGGCAACGAGAGCAA | |
| | | | AACTCCAATACAAACAAACAAAC | |
| | | | AAACACCTGTGCTAGGTCAGTCTGGCACGTAAGATGAACATCCCTAC | |
| | | | CAACACAGAGCTCACCATCTCTT | |
| | | | ATACTTAAGTGAAAAACATGGGGAAGGGGAAAGGGGAATGGCTGCTT | |
| | | | TTGATATGTTCCCTGACACATAT | |
| | | | CTTGAATGGAGACCTCCCTACCAAGTGATGAAAGTGTTGAAAAACTT | |
| | | | AATAACAAATGCTTGTTGGGCAA | |
| | | | GAATGGGATTGAGGATTATCTTGTCTCAGAAAGGCATTGTGAAGGAA | |
| | | | TTGAGCCAGATCTCTCTCCCTAC | |
| | | | TGCAAAACCCTATTGTAGTAAAAAAGTCTTCTTTACTATCTTAATAA | |
| | | | AACAGATATTGTGAGATTCAAAA | |
| | | | AAAAAAAAAAAA | |
| 229152_ | chromosome | tgaagaaagttctcctcctgatca | >gi\|50428928\|ref\|NM_152997.2\|Homo sapiens | 154.05 |
| at | 4 open | cagccatcttggcagtggctgttg | chromosome 4 open reading frame 7 (C4orf7), | |
| | reading | gtttcccagtct | mRNA | |
| | frame 7 | ctcaagaccaggaacgagaaaaaa | TAAAACAGCTACAATATTCCAGGGCCAGTCACTTGCCATTTCTCATA | |
| | | gaagtatcagtgacagcgatgaat | ACAGCGTCAGAGAGAAAGAACTG | |
| | | tagcttcagggttttttgtgttcc | ACTGAAACGTTTGAGATGAAGAAAGTTCTCCTCCTGATCACAGCCAT | |
| | | cttacccatatccatttcgcccac | CTTGGCAGTGGCTGTTGGTTTCC | |
| | | ttccaccaattccatttccaagat | CAGTCTCTCAAGACCAGGAACGAGAAAAAAGAAGTATCAGTGACAGC | |
| | | ttccatggtttagacgtaattttc | GATGAATTAGCTTCAGGGTTTTT | |
| | | ctattccaatacctgaatctgccc | TGTGTTCCCTTACCCATATCCATTTCGCCCACTTCCACCAATTCCAT | |
| | | ctacaactcccc | TTCCAAGATTTCCATGGTTTAGA | |
| | | ttcctagcgaaaagtaaacaagaa | CGTAATTTTCCTATTCCAATACCTGAATCTGCCCCTACAACTCCCCT | |
| | | ggaaaagtcacgataaacctggtc | TCCTAGCGAAAAGTAAACAAGAA | |
| | | acctgaaattgaaattgagccact | GGAAAAGTCACGATAAACCTGGTCACCTGAAATTGAAATTGAGCCAC | |
| | | tccttgaagaatcaaaattcctgt | TTCCTTGAAGAATCAAAATTCCT | |
| | | taataaaagaaaaacaaatgtaat | GTTAATAAAAGAAAAACAAATGTAATTGAAATAGCACACAGCATTCT | |
| | | tgaaatagcacacagcattctcta | CTAGTCAATATCTTTAGTGATCT | |
| | | gtcaatatctttagtgatcttctt | TCTTTAATAAACTTGAAAGCAAAGATTTTGGTTTCTTAATTTCCACA | |
| | | taata | AA | |
| | | [Seq Id No 79] | | |
| 236203_ | Major histo- | atgtcaggtttgtacctaccacat | >gi\|52426772\|ref\|NM_002122.3\|Homo sapiens | 38.77 |
| at | compatibility | ttaaaatagggacttgaagaatta | major histocompatibility complex, class II, DQ | |
| | complex, | aacattttatta | alpha 1 (HLA-DQA1), mRNA | |
| | class II, | caaatgaagcacttcatgcacaga | ACAATTACTCTACAGCTCAGAACACCAACTGCTGAGGCTGCCTTGGG | |
| | DQ alpha 1 | ctggcacatagtaagtagtcgata | AAGAGGATGATCCTAAACAAAGC | |
| | | ggtgttaacaat | TCTGCTGCTGGGGGCCCTCGCTCTGACCACCGTGATGAGCCCCTGTG | |
| | | ttgtgttattgttattttctggag | GAGGTGAAGACATTGTGGCTGAC | |
| | | tccaactaacaaatcccacagtga | CACGTTGCCTCTTGTGGTGTAAACTTGTACCAGTTTTACGGTCCCTC | |
| | | atgacatcacag | TGGCCAGTACACCCATGAATTTG | |
| | | ggatgcaaccaacaagatccagaa | ATGGAGATGAGCAGTTCTACGTGGACCTGGAGAGGAAGGAGACTGCC | |
| | | tatggaaacttctactagataaac | TGGCGGTGGCCTGAGTTCAGCAA | |
| | | aactccatttct | ATTTGGAGGTTTTGACCCGCAGGGTGCACTGAGAAACATGGCTGTGG | |
| | | tcagcaacaattcaagagagagag | CAAAACACAACTTGAACATCATG | |
| | | agaagagaagctatacattttaaa | ATTAAACGCTACAACTCTACCGCTGCTACCAATGAGGTTCCTGAGGT | |
| | | aggctgaagaaatatatgaaccaa | CACAGTGTTTTCCAAGTCTCCCG | |
| | | atttgtatgaggcaatcagaaaaa | TGACACTGGGTCAGCCCAACACCCTCATTTGTCTTGTGGACAACATC | |
| | | ctgacaccgactgtattaaggaat | TTTCCTCCTGTGGTCAACATCAC | |
| | | tatctaattttagtgtggtaatga | ATGGCTGAGCAATGGGCAGTCAGTCACAGAAGGTGTTTCTGAGACCA | |
| | | gattgctgttatgttttctaa | GCTTCCTCTCCAAGAGTGATCAT | |
| | | [Seq Id No 80] | TCCTTCTTCAAGATCAGTTACCTCACCTTCCTCCCTTCTGCTGATGA | |
| | | | GATTTATGACTGCAAGGTGGAGC | |
| | | | ACTGGGGCCTGGACCAGCCTCTTCTGAAACACTGGGAGCCTGAGATT | |
| | | | CCAGCCCCTATGTCAGAGCTCAC | |
| | | | AGAGACTGTGGTCTGTGCCCTGGGGTTGTCTGTGGGCCTCATGGGCA | |
| | | | TTGTGGTGGGCACTGTCTTCATC | |
| | | | ATCCAAGGCCTGCGTTCAGTTGGTGCTTCCAGACACCAAGGGCCATT | |
| | | | GTGAATCCCATCCTGGAAGGGAA | |
| | | | GGTGCATCGCCATCTACAGGAGCAGAAGAATGGACTTGCTAAATGAC | |
| | | | CTAGCACTATTCTGTGGCCCGAT | |
| | | | TTATCATATCCCTTTTCTCCTCCAAATATTTCTCCTCTCACCTTTTC | |
| | | | TCTGGGACTTAAGCTGCTATATC | |
| | | | CCCTCAGAGCTCACAAATGCCTTTACATTCTTTCCCTGACCTCCTGA | |
| | | | TTTTTTTTTTCTTTTCTCAAATG | |
| | | | TTACCTACAAAGACATGCCTGGGGTAAGCCACCCGGCTACCTAATTC | |
| | | | CTCAGTAACCTCCATCTAAAATC | |
| | | | TCCAAGGAAGCAATAAATTCCTTTTATGAGATCTATGTCAAATTTTT | |
| | | | CCATCTTTCATCCAGGGCTGACT | |
| | | | GAAACTATGGCTAATAATTGGGGTACTCTTATGTTTCAATCCAATTT | |
| | | | AACCTCATTTCCCAGATCATTTT | |
| | | | TCATGTCCAGTAACACAGAAGCCACCAAGTACAGTATAGCCTGATAA | |
| | | | TATGTTGATTTCTTAGCTGACAT | |
| | | | TAATATTTCTTGCTTCCTTGTGTTCCCACCCTTGGCACTGCCACCCA | |
| | | | CCCCTCAATTCAGGCAACAATGA | |
| | | | AATTAATGGATACCGTCTGCCCTTGGCCCAGAATTGTTATAGCAAAA | |
| | | | ATTTTAGAACCAAAAAATAAGTC | |
| | | | TGTACTAATTTCAATGTGGCTTTTAAAAGTATGACAGAGAAATAAGT | |
| | | | TAGGATAAAGGAAATTTGAATCT | |
| | | | CA | |
| 236280_ | Transcribe | aaaagatctctcactgggaaaaga | >gi\|3807951\|gb\|AI225238.1\|AI225238 qx12c04.x1 | 77.63 |
| at | d locus | aaaagttatgcatttataaagtaa | NCI_CGAP_Lym12 Homo sapiens cDNA clone | |
| | | ttaaactggttt | IMAGE: 2001126 3′, mRNA sequence | |
| | | tccttgtactttattaatctgaat | AGCGGCCGCCCTTTTTTTTTTTTTTTGAATGTTTTTATATTTTTATG | |
| | | ctaatggcacttccttacgagggt | TTTTTCTCTCTCACCACATTAGA | |
| | | tttcagatgtgc | GATTCACATGTCAAAAAAAAAGTTCAAAGATTCCACTTAAATGGTAG | |
| | | ttgtagttaatggcaacattatca | TCAAAATTAAAAGCATGTTGTTT | |
| | | gaatgactacacagacagtcctac | AATTTGCCAACTATTCCATAAATCCTGTGCTTGAAGCACTACCTTTT | |
| | | tctgaggagatg | ATCAACCACTTGAAAGGAAGGAA | |
| | | actttggaagaaacccatttggaa | TATGAAGTGTGACTCTTGAGGATTGCAGTTCCATTTCTCCACAGACA | |
| | | ctacacaccctgctatgtctgtgg | TAGCAGGGTGTGTAGTTCCAAAT | |
| | | agaaatggaact | GGGTTTCTTCCAAAGTCATCTCCTCAGAGTAGGACTGTCTGAGTAGT | |
| | | gcaatcctcaagagtcacacttca | CATTCTGATAATGTTGCCATTAA | |
| | | tattccttcctttcaagtggttga | CTACAAGCACATCTGAGAACCCTCGTAAGGAAGTGCCATTAGATTCA | |
| | | taaaaggtagtg | GA | |
| | | cttcaagcacaggatttatggaat | | |
| | | agttggcaaattaaacaacatgct | | |
| | | ttttattttgac | | |
| | | tacca | | |
| | | [Seq Id No 81] | | |
| 239237_ | Amphiphysin | ttatcaccattaatccatgccagt | >gi\|51466133\|ref\|XM_A99518.1\|PREDICTED: Homo | 25.87 |
| at | (Stiff-Man | tatgtacagttttgcatgtttgtt | sapiens LOC442534 (LOC442534), mRNA | |
| | syndrome | ttttattttact | GGCCCAGCCCTAGGAGGGGGCAGTCCCACAGCAGGCTCACACCCTGC | |
| | with | cttttctctccttcattccctatt | CCTTCAGAGTTGCAGCAGCTAAT | |
| | breast | cctgttcccccataggtgcccagt | GCTGATGCCAGGACCTTGTCCTGCCAGGAAGCCTCTGATTAGGGCTG | |
| | cancer | ctaatgtatttg | TGAACCAACTTCCTTTTCACATC | |
| | 128 kDa | atatctgtccttagagcctccgta | TGCAAGGACTCTGCTCTAAGCTCAAATTTTGCAGTTTCTGCTGTCAT | |
| | autoantigen) | tctgtgaagactagagtcatgcac | CACAGCTGGGAATGAGAAAGGGA | |
| | | ttcatcacattt | AGGGGCTCCAGTTGGTCTCCCTCTGCCTCTGGAGCACACGTGGAGGA | |
| | | cagtaacgatgggccgcatgtacc | CATGAGGCTTCCGCAGGAACAGC | |
| | | acagtcccatgagatgatagaggt | TCTGAAAGCTGAGTTCTCGGGTCCCTGTGAAGGAGGGAAGCTTGCCT | |
| | | gcagaaaaattcctgtcatctagt | GCCTTGGAGCGTGAAAAAGTCTT | |
| | | gacatcgtagccatcataacatnn | GCTGGACAGAAGTCTCACGGTTTAAAACTGGGAAGGGAGAGGATTGA | |
| | | caacacgactctcatttgtggtga | GATCACACTAGAATGCACAGAAA | |
| | | ccctggtgtacacaaacctac | ATTCTT | |
| | | [Seq Id No 82] | | |
| 244413_ | dendritic | atggctggagatgtagtctacgct | >gi\|40548404\|ref\|NM_172004.2\|Homo sapiens | 12.75 |
| at | cell- | gacatcaaaactgttcggacttcc | dendritic cell-associated lectin-1 (DCAL1), | |
| | associated | ccgttagaactc | mRNA | |
| | lectin-1 | gcgtttccacttcagagatctgtt | ATGGTTAGTAATTTCTTCCATGTCATACAAGTATTCGAGAAATCTGC | |
| | | tctttcaacttttctactgtccat | TACCTTGATTAGTAAGACTGAAC | |
| | | aaatcatgtcct | ACATTGGTTTTGTCATTTATTCATGGAGGAAGTCCACCACCCACTTG | |
| | | gccaaagactggaaggtgcataag | GGGAGCAGAAGGAAATTTGCCAT | |
| | | ggaaaatgttactggattgctgaa | CTCAATTTACTTATCAGAAGTTTCTTTGCAGAAATATGATTGTCCCT | |
| | | actaagaaatcttggaacaaaagt | TCAGTGGGACATCATTTGTGGTC | |
| | | caaaatgactgtgccataaacaat | TTCTCTCTCTTTTTGATCTGTGCAATGGCTGGAGATGTAGTCTACGC | |
| | | tcatatctcatggtgattcaagac | TGACATCAAAACTGTTCGGACTT | |
| | | attactgctatggtgagatttaac | CCCCGTTAGAACTCGCGTTTCCACTTCAGAGATCTGTTTCTTTCAAC | |
| | | atttagaggtgacagcatccccca | TTTTCTACTGTCCATAAATCATG | |
| | | cactggcagtga | TCCTGCCAAAGACTGGAAGGTGCATAAGGGAAAATGTTACTGGATTG | |
| | | attttttgtgctacaaacttggca | CTGAAACTAAGAAATCTTGGAAC | |
| | | aaagtctgtgaaaagaagtttcaa | AAAAGTCAAAATGACTGTGCCATAAACAATTCATATCTCATGGTGAT | |
| | | cttcatgtgtta | TCAAGACATTACTGCTATGGTGA | |
| | | ttaact | GATTTAACATTTAGAGGTGACAGCATCCCCCACACTGGCAGTGAATT | |
| | | [Seq Id No 83] | TTTTGTGCTACAAACTTGGCAAA | |
| | | | AGTCTGTGAAAAGAAGTTTCAACTTCATGTGTTATTAACTATACAAA | |
| | | | TATTAGTTGAATGAATTGTTGAA | |
| | | | TTACAAAAAAAAAAAAAAA | |
| 34210_ | CD52 | Taatcggctcactataggaatttg | >gi\|68342029\|ref\|NM_001803.2\|Homo sapiens CD52 | 31.41 |
| at | antigen | cntcgaggccaagattcgnacgag | molecule (CD52), mRNA | |
| | (CAMPATH-1 | aagaagcctccagacagccctgag | CTCCTGGTTCAAAAGCAGCTAAACCAAAAGAAGCCTCCAGACAGCCC | |
| | antigen) | atcacctaaaaagctgctaccaag | TGAGATCACCTAAAAAGCTGCTA | |
| | | acagccacgaagatcctaccaaaa | CCAAGACAGCCACGAAGATCCTACCAAAATGAAGCGCTTCCTCTTCC | |
| | | tgaagcgcttcctcttcctcctac | TCCTACTCACCATCAGCCTCCTG | |
| | | tcaccatcagcctcctggttatgg | GTTATGGTACAGATACAAACTGGACTCTCAGGACAAAACGACACCAG | |
| | | tacagatacaaactggactctcag | CCAAACCAGCAGCCCCTCAGCAT | |
| | | gacaaaacgacaccagccaaacca | CCAGCAACATAAGCGGAGGCATITTCCTTTTCTTCGTGGCCAATGCC | |
| | | gcagcccctcagcatacagcagca | ATAATCCACCTCTTCTGCTTCAG | |
| | | tgagcggaggcattttccttttct | TTGAGGTGACACGTCTCAGCCTTAGCCCTGTGCCCCCTGAAACAGCT | |
| | | tcgtggccaatgccataatccacc | GCCACCATCACTCGCAAGAGAAT | |
| | | tcttctgcttcagttgaggtgaca | CCCCTCCATCTTTGGGAGGGGTTGATGCCAGACATCACCAGGTTGTA | |
| | | cgtctcagccttagccctgtgccc | GAAGTTGACAGGCAGTGCCATGG | |
| | | cctgaaacannnnnnnnnnnnnnn | GGGCAACAGCCAAAATAGGGGGGTAATGATGTAGGGGCCAAGCAGTG | |
| | | nnnnagagaatcccctccatcttt | CCCAGCTGGGGGTCAATAAAGTT | |
| | | gggaggggttgatgccagacatca | ACCCTTGTACTTGAAAAAAAAAAAAAAAAAAAA | |
| | | ccaggttgtagaagttgacaggca | | |
| | | gtgccatggggncaacagccaaaa | | |
| | | taggggggtaatgatgtaggggcc | | |
| | | aagcagtgcccagctgggggtcaa | | |
| | | taaagttacccttgtacttg | | |
| | | [Seq Id No 84] | | |

TABLE 3A
List of 13 probe sets suitable for identify the genes listed in Table 3

| Probe | Gene | | |
|---|---|---|---|
| Set ID | Symbol | Gene Title | Gene Sequences |
| 204661_at | CD52 | CD52 molecule /// | Acagccacgaagatcctaccaaaatgaagcgcttcctcttcctcctactcaccatcagcctcc |
| | | CD52 molecule | tggttatggtacagatacaaactggactctcaggacaaaacgacaccagccaaaccagcagcc |
| | | | cctcagcatccagcagcatgagcggaggcattttccttttcttcgtggccaatgccataatcc |
| | | | acctcttctgcttcagttgaggtgacacgtctcagccttagccctgtgccccctgaaacagct |
| | | | gccaccatcactcgcaagagaatcccctccatctttgggaggggttgatgccagacatcacca |
| | | | ggttgtagaagttgacaggcagtgccatgggggcaacagccaaaataggggggtaatgatgta |
| | | | ggggccaagc |
| | | | [Seq Id No 85] |
| 205890_s_at | UBD | gamma- | Gatcttaaagccacggagaagcctctcatcttatggcattgacaaagagaagaccatccacct |
| | | aminobutyric acid | taccctgaaagtggtgaagcccagtgatgaggagctgcccttgtttcttgtggagtcaggtga |
| | | (GABA) B | tgaggcaaagaggcacctcctccaggtgcgaaggtccagctcagtggcacaagtgaaagcaat |
| | | receptor, 1 /// | gatcgagactaagacgggtataatccctgagacccagattgtgacttgcaatggaaagagact |
| | | ubiquitin D | ggaagatgggaagatgatggcagattacggcatcagaaagggcaacttactcttcctggcatc |
| | | | ttattgtattggagggtgaccaccctggggatggggtgttggcaggggtcaaaaagcttattt |
| | | | cttttaatctcttactcaacgaacacatcttctgatgatttcccaaaattaatgagaatgaga |
| | | | tgagtagagtaagatttgggtgggatgggtaggatgaagtatattgcccaactctatgtttct |
| | | | ttga |
| | | | [Seq Id No 86] |
| 206118_at | STAT4 | signal transducer | Gctgacatcctgcgagactacaaagttattatggctgaaaacattcctgaaaaccctctgaag |
| | | and activator of | tacctatatcctgacattcccaaagacaaagccttcggtaaacactacagctctcagccttgc |
| | | transcription 4 | gaagtttcaagaccaacagaaaggggtgacaaaggttatgttccttctgtttttatccccatc |
| | | | tcaacaatccgaagtgattcaacagagccacattctccatcagaccttcttcccatgtctcca |
| | | | agtgtgtatgcggtgttgagagaaaacctgagtcccacaacaattgaaactgcaatgaagtct |
| | | | ccttattctgctgaatgacaggataaactctgacgcaccaagaaaggaagcaaatgaaaaagt |
| | | | ttaaagactgttctttgcccaataaccacattttatttcttcagctttgtaaataccaggttc |
| | | | taggaaatgtttgacatctgaagctctcttcacactcccgtggcactcctcaattgggag |
| | | | [Seq Id No 87] |
| 206666_at | GZMK | granzyme K | aaacctctcttagatctggaaccaaatgcaaggttactggctggggagccaccgatccagatt |
| | | (granzyme 3; | cattaagaccttctgacaccctgcgagaagtcactgttactgtcctaagtcgaaaactttgca |
| | | tryptase II) /// | acagccaaagttactacaacggcgacccttttatcaccaaagacatggtctgtgcaggagatg |
| | | granzyme K | ccaaaggccagaaggattcctgtaagggtgactcagggggccccttgatctgtaaaggtgtct |
| | | (granzyme 3; | tccacgctatagtctctggaggtcatgaatgtggtgttgccacaaagcctggaatctacaccct |
| | | tryptase II) | gttaaccaagaaataccagacttggatcaaaagcaaccttgtcccgcctcatacaaattaagtt |
| | | | acaaataattttattggatgcacttgcttcttttttcctaatatgctcgcaggttagagttggg |
| | | | tgtaagtaaagcagagcacatatggggtccatttttgcacttgta |
| | | | [Seq Id No 88] |
| 207651_at | GPR171 | G protein-coupled | Ttgccttgtaattcgacagctctacagaaacaaagataatgaaaattacccaaatgtgaaaaa |
| | | receptor 171 | ggctctcatcaacatacttttagtgaccacgggctacatcatatgctttgttccttaccacat |
| | | | tgtccgaatcccgtataccctcagccagacagaagtcataactgattgctcaaccaggatttc |
| | | | actcttcaaagccaaagaggctacactgctcctggctgtgtcgaacctgtgctttgatcctat |
| | | | cctgtactatcacctctcaaaagcattccgctcaaaggtcactgagacttttgcctcacctaa |
| | | | agagaccaaggctcagaaagaaaaattaagatgtgaaaataatgcataaaagacaggattttt |
| | | | tgtgctaccaattctggccttactgga |
| | | | [Seq Id No 89] |
| 210038_at | PRKCQ | protein kinase C, | Aatccattcatcctgattgggcatgaaatccatggtcaagaggacaagtggaaagtgagaggg |
| | | theta | aaggtttgctagacaccttcgcttgttatcttgtcaagatagaaaagatagtatcatttcacc |
| | | | cttgccagtaaaaacctttccatccacccattctcagcagactccagtattggcacagtcact |
| | | | cactgccattctcacactataacaagaaaagaaatgaagtgcataagtctcctgggaaaagaa |
| | | | ccttaaccccttctcgtgccatgactggtgatttcatgactcataagcccctccgtaggcatc |
| | | | attcaagatcaatggcccatgcatgctgtttgcagca |
| | | | [Seq Id No 90] |
| 210972_x_at | TRA@ | T cell receptor | Ggaacaagacttcaggtcacgctcgatatccagaaccctgaccctgccgtgtaccagct |
| | /// | alpha locus /// T | gagagactctaaatccagtgacaagtctgtctgcctattcaccgattttgattctcaaa |
| | TRDV2 | cell receptor | caaatgtgtcacaaagtaaggattctgatgtgtatatcacagacaaaactgtgctagac |
| | /// | delta variable 2 | atgaggtctatggacttcaagagcaacagtgctgtggcctggagcaacaaatctgactt |
| | TRAV20 | /// T cell | tgcatgtgcaaacgccttcaacaacagcattattccagaagacaccttcttccccagcc |
| | /// | receptor alpha | cagaaagttcctgtgatgtcaagctggtcgagaaaagctttgaaacagatacgaaccta |
| | TRAC | variable 20 /// T | aactttcaaaacctgtcagtgattgggttccgaatcctcctcctgaaagtggccgggtt |
| | | cell receptor | taatctgctcatgacgctgcggctgtggtccagctgagatctgcaagattgtaagacag |
| | | alpha constant | cctgtgctccct |
| | | | [Seq Id No 91] |
| 211144_x_at | TRGC2 | T cell receptor | Aaatgatacactactgctgcagctcacaaacacctctgcatattacatgtacctcctcct |
| | /// | gamma constant 2 | gctcctcaagagtgtggtctattttgccatcatcacctgctgtctgcttggaagaacggc |
| | TRGV2 | /// T cell | tttctgctgcaatggagagaaatcataacagacggtggcacaaggaggccatcttttcct |
| | /// | receptor gamma | catcggttattgtccctagaagcgtcttctgaggatctagttgggctttctttctgggtt |
| | TRGV9 | variable 2 /// T | tgggccatttcagttctcatgtgtgtactattctatcattattgtataatggttttcaaa |
| | /// | cell receptor | ccagtgggcacacagagaacctcagtctgtaataacaatgaggaatagccatggcgatct |
| | TARP | gamma variable 9 | ccagcaccaatctctccatgttttccacagctcctccagccaacccaaatagcgcctgct |
| | /// | /// TCR gamma | atagtgtagacagcctgcggcttctagccttgtccctctcttagtgttctttaatcagat |
| | LOC642 | alternate reading | aactgcctggaagcctttcattttacacgccc |
| | 083 | frame protein III | [Seq Id No 92] |
| | | hypothetical | |
| | | protein L0C642083 | |
| 211796_s_at | TRBV21-1 | T cell receptor | gccatcagaagcagagatctcccacacccaaaaggccacactggtgtgcctggccacaggtt |
| | /// | beta variable 21- | tctaccccgaccacgtggagctgagctggtgggtgaatgggaaggaggtgcacagtggggtc |
| | TRBV19 | 1 /// T cell | agcacagacccgcagcccctcaaggagcagcccgccctcaatgactccagatactgcctgag |
| | /// | receptor beta | cagccgcctgagggtctcggccaccttctggcagaacccccgcaaccacttccgctgtcaag |
| | TRBV5- | variable 19 /// T | tccagttctacgggctctcggagaatgacgagtggacccaggatagggccaaacctgtcacc |
| | 4 /// | cell receptor | cagatcgtcagcgccgaggcctggggtagagcagactgtggcttcacctccgagtcttacca |
| | TRBV3- | beta variable 5-4 | gcaaggggtcctgtctgccaccatcctctatgagatcttgctagggaaggccaccttgtatg |
| | 1 /// | /// T cell | ctgtgctggtcagtgccctcgtgctgatggccatggtcaagagaaagga |
| | TRBC1 | receptor beta | [Seq Id No 93] |
| | | variable 3-1 /// | |
| | | T cell receptor | |
| | | beta constant 1 | |
| | | /// similar to T- | |
| | | cell receptor | |
| | | beta chain V | |
| | | region CTL-L17 | |
| | | precursor | |
| 213193_x_at | TRBV19 | T cell receptor | Tgactccagatactgcctgagcagccgcctgagggtctcggccaccttctggcagaaccg |
| | /// | beta variable 19 | cccgcaaccacttccctgtcaagtccagttctacgggctctcggagaatgacgagtggac |
| | TRBC1 | /// T cell | ccaggatagggccaaacccgtcacccagatcgtcagcgccgaggcctggggtagagcaga |
| | | receptor beta | ctgtggctttacctcggtgtcctaccagcaaggggtcctgtctgccaccatcctctatga |
| | | variable 19 /// T | gatcctgctagggaaggccaccctgtatgctgtgctggtcagcncccttgtgttgatggc |
| | | cell receptor | catggtcaagagaaaggatttctgaaggcagccctggaagtggagttaggagcttctaac |
| | | beta constant 1 | ccgtcatggtttcaatacacattcttcttttgccagcgcttctgaagagctgctctcacc |
| | | /// T cell | tctctgcatcccaatagatatccccctatgtgcatgcacacctgcacactcacggctgaa |
| | | receptor beta | atctccctaacccagggggaccttagcatgcctaagtga |
| | | constant 1 | [Seq Id No 94] |
| 213539_at | CD3D | CD3d molecule, | gggaacactgctctcagacattacaagactggacctgggaaaacgcatcctggacccacg |
| | | delta (CD3-TCR | aggaatatataggtgtaatgggacagatatatacaaggacaaagaatctaccgtgcaagt |
| | | complex) | tcattatcgaatgtgccagagctgtgtggagctggatccagccaccgtggctggcatcat |
| | | | tgtcactgatgtcattgccactctgctccttgctttgggagtcttctgctttgctggaca |
| | | | tgagactggaaggctgtctggggctgccgacacacaagctctgttgaggaatgaccaggt |
| | | | ctatcagcccctccgagatcgagatgatgctcagtacagccaccttggaggaaactgggc |
| | | | tcggaacaagtgaacctgagactggtggcttctagaagcagccattaccaactgtacct |
| | | | [Seq Id No 95] |
| 217147_s_at | TRAT1 | T cell receptor | Tctcctttctcaccaatgggcaatagcccataattgaaataaatttctgattgaaaggt |
| | | associated | ataggaaacattaaaatgcattactaagagaagtaatataattttcttacaaagtattt |
| | | transmembrane | ttcccaaagatagctttactatttcaaaaattgtcaaattaatgcatgctccttacaac |
| | | adaptor 1 | aaacaaatatcaaaaagagtttaggaattctactagccagagatagtcacttggagaaa |
| | | | ctttctatatatccttctaaatatttttctgggcatgctcatgtatgtacatcagttgt |
| | | | ttctttttattttgaaccaaaaatgtggtttcttttgtacacattacttaaactttctt |
| | | | tccagtcaacaatatattgtggatttattttcactgttatatttaactatatataaata |
| | | | cgcatatattgtaattttaatgtctgcttagcaccccactgataaccaaatcacag |
| | | | [Seq Id No 96] |
| 34210_at | CD52 | CD52 molecule | Taatcggctcactataggaatttgcntcgaggccaagattcgnacgagnnngttcaaaa |
| | | | gcagctaaaccaaaagaagcctccagacagccctgagatcacctaaaaagctgctacca |
| | | | agacagccacgaagatcctaccaaaatgaagcgcttcctcttcctcctactcaccatca |
| | | | gcctcctggttatggtacagatacaaactggactctcaggacaaaacgacaccagccaa |
| | | | accagcagcccctcagcatccagcagcatgagcggaggcattttccttttcttcgtggc |
| | | | caatgccataatccacctcttctgcttcagttgaggtgacacgtctcagccttagccct |
| | | | gtgccccctgaaacannnnnnnnnnnnnnnnnnnagagaatcccctccatctttgggag |
| | | | gggttgatgccagacatcaccaggttgtagaagttgacaggcagtgccatggggncaac |
| | | | agccaaaataggggggtaatgatgtaggggccaagcagtgcccagctgggggtcaataa |
| | | | agttacccttgtacttg |
| | | | [Seq Id No 97] |

TABLE 4A
Table linking probe set id and the gene list of Table 4.

| Probe Set ID | Gene Symbol | Gene Name |
|---|---|---|
| 218802_at | FLJ20647 | NA (not applicable) |
| 224774_s_at | NAV1 | neuron navigator 1 |
| 207651_at | GPR171 | G protein-coupled receptor 171 |
| 205392_s_at | CCL14 | chemokine (C-C motif) ligand 14 |
| 1555229_a_at | C1S | complement component 1, s subcomponent |
| 209774_x_at | CXCL2 | chemokine (C—X—C motif) ligand 2 |
| 211796_s_at | TRBV3-1 | T cell receptor beta variable 3-1 |
| 210972_x_at | TRDV2 | T cell receptor delta variable 2 |
| 210251_s_at | RUFY3 | RUN and FYVE domain containing 3 |
| 225502_at | DOCK8 | dedicator of cytokinesis 8 |
| 204224_s_at | GCH1 | GTP cyclohydrolase 1 (dopa-responsive dystonia) |
| 218950_at | CENTD3 | centaurin, delta 3 |
| 218322_s_at | ACSL5 | acyl-CoA synthetase long-chain family member 5 |
| 228094_at | AMICA1 | adhesion molecule, interacts with CXADR antigen 1 |
| 204116_at | IL2RG | interleukin 2 receptor, gamma (severe combined |
| | | immunodeficiency) |
| 202643_s_at | TNFAIP3 | tumor necrosis factor, alpha-induced protein 3 |
| 209606_at | PSCDBP | pleckstrin homology, Sec7 and coiled-coil domains, binding |
| | | protein |
| 205225_at | ESR1 | estrogen receptor 1 |
| 213193_x_at | TRBC1 | T cell receptor beta constant 1 |
| 204661_at | CD52 | CD52 antigen (CAMPATH-1 antigen) |
| 216920_s_at | LOC442535 | NA |
| 210915_x_at | TRBV19 | T cell receptor beta variable 19 |
| 226218_at | IL7R | interleukin 7 receptor |
| 209670_at | TRAC | T cell receptor alpha constant |
| 209949_at | NCF2 | neutrophil cytosolic factor 2 (65 kDa, chronic granulomatous |
| | | disease, autosomal 2) |
| 226697_at | LOC92689 | NA |
| 206666_at | GZMK | c(“granzyme K (granzyme 3”, “tryptase II)”) |
| 235831_at | NA | NA |
| 1555630_a_at | RAB34 | RAB34, member RAS oncogene family |
| 207977_s_at | DPT | dermatopontin |
| 1558290_a_at | PVT1 | Pvt1 oncogene homolog, MYC activator (mouse) |
| 215806_x_at | TRGC2 | T cell receptor gamma constant 2 |
| 64064_at | GIMAP5 | GTPase, IMAP family member 5 |
| 34210_at | CD52 | CD52 antigen (CAMPATH-1 antigen) |
| 213539_at | CD3D | CD3d antigen, delta polypeptide (TiT3 complex) |
| 232313_at | TMEM132C | transmembrane protein 132C |
| 201502_s_at | NFKBIA | nuclear factor of kappa light polypeptide gene enhancer in B- |
| | | cells inhibitor, alpha |
| 211902_x_at | TRA@ | T cell receptor alpha locus |
| 217147_s_at | TRAT1 | T cell receptor associated transmembrane adaptor 1 |
| 211144_x_at | LOC442535 | NA |
| 224710_at | RAB34 | RAB34, member RAS oncogene family |
| 209795_at | CD69 | CD69 antigen (p60, early T-cell activation antigen) |
| 232843_s_at | DOCK8 | dedicator of cytokinesis 8 |
| 218805_at | GIMAP5 | GTPase, IMAP family member 5 |
| 204057_at | IRF8 | interferon regulatory factor 8 |
| 214470_at | KLRB1 | killer cell lectin-like receptor subfamily B, member 1 |
| 236280_at | NA | NA |
| 201474_s_at | ITGA3 | integrin, alpha 3 (antigen CD49C, alpha 3 subunit of VLA-3 |
| | | receptor) |
| 235421_at | MAP3K8 | mitogen-activated protein kinase kinase kinase 8 |
| 228532_at | C1orf162 | chromosome 1 open reading frame 162 |
| 205890_s_at | UBD | ubiquitin D |
| 209813_x_at | TRGV9 | T cell receptor gamma variable 9 |
| 224772_at | NAV1 | neuron navigator 1 |
| 224451_x_at | ARHGAP9 | Rho GTPase activating protein 9 |
| 226117_at | TIFA | NA |
| 213068_at | DPT | dermatopontin |
| 1569942_at | NA | NA |
| 219243_at | GIMAP4 | GTPase, IMAP family member 4 |
| 202957_at | HCLS1 | hematopoietic cell-specific Lyn substrate 1 |
| 218764_at | PRKCH | protein kinase C, eta |
| 206118_at | STAT4 | signal transducer and activator of transcription 4 |
| 236203_at | HLA-DQA1 | major histocompatibility complex, class II, DQ alpha 1 |
| 206170_at | ADRB2 | adrenergic, beta-2-, receptor, surface |
| 239237_at | NA | NA |
| 214450_at | CTSW | cathepsin W (lymphopain) |
| 201497_x_at | MYH11 | myosin, heavy polypeptide 11, smooth muscle |
| 219777_at | GIMAP6 | GTPase, IMAP family member 6 |
| 211654_x_at | HLA-DQB1 | major histocompatibility complex, class II, DQ beta 1 |
| 205758_at | CD8A | CD8 antigen, alpha polypeptide (p32) |
| 202644_s_at | TNFAIP3 | tumor necrosis factor, alpha-induced protein 3 |
| 1558034_s_at | CP | ceruloplasmin (ferroxidase) |
| 223235_s_at | SMOC2 | SPARC related modular calcium binding 2 |
| 232234_at | C20orf24 | chromosome 20 open reading frame 24 |
| 1559584_a_at | C16orf54 | chromosome 16 open reading frame 54 |
| 205831_at | CD2 | CD2 antigen (p50), sheep red blood cell receptor |
| 203812_at | SLIT3 | slit homolog 3 (Drosophila) |
| 222780_s_at | BAALC | brain and acute leukemia, cytoplasmic |
| 218145_at | TRIB3 | tribbles homolog 3 (Drosophila) |
| 242881_x_at | LOC440160 | NA |
| 1558972_s_at | C6orf190 | chromosome 6 open reading frame 190 |
| 229723_at | TAGAP | T-cell activation GTPase activating protein |
| 235391_at | FAM92A1 | family with sequence similarity 92, member A1 |
| 219938_s_at | PSTPIP2 | proline-serine-threonine phosphatase interacting protein 2 |
| 212587_s_at | PTPRC | protein tyrosine phosphatase, receptor type, C |
| 210982_s_at | HLA-DRA | major histocompatibility complex, class II, DR alpha |
| 211200_s_at | EFCAB2 | EF-hand calcium binding domain 2 |
| 210260_s_at | TNFAIP8 | tumor necrosis factor, alpha-induced protein 8 |
| 228869_at | SLIC1 | NA |
| 205987_at | CD1C | CD1c antigen |
| 213888_s_at | TRAF3IP3 | TRAF3 interacting protein 3 |
| 212671_s_at | HLA-DQA1 | major histocompatibility complex, class II, DQ alpha 1 |
| 242986_at | NAV1 | neuron navigator 1 |
| 212999_x_at | HLA-DQB1 | major histocompatibility complex, class II, DQ beta 1 |
| 212588_at | PTPRC | protein tyrosine phosphatase, receptor type, C |
| 212592_at | IGJ | immunoglobulin J polypeptide, linker protein for |
| | | immunoglobulin alpha and mu polypeptides |
| 203471_s_at | PLEK | pleckstrin |
| 209671_x_at | TRA@ | T cell receptor alpha locus |
| 228054_at | TMEM44 | transmembrane protein 44 |
| 216191_s_at | TRA@ | T cell receptor alpha locus |
| 205419_at | EBI2 | Epstein-Barr virus induced gene 2 (lymphocyte-specific G |
| | | protein-coupled receptor) |
| 220330_s_at | SAMSN1 | SAM domain, SH3 domain and nuclear localisation signals, 1 |
| 213008_at | KIAA1794 | KIAA1794 |
| 201425_at | ALDH2 | aldehyde dehydrogenase 2 family (mitochondrial) |
| 1552613_s_at | CDC42SE2 | CDC42 small effector 2 |
| 205696_s_at | GFRA1 | GDNF family receptor alpha 1 |
| 211339_s_at | ITK | IL2-inducible T-cell kinase |
| 208894_at | HLA-DRA | major histocompatibility complex, class II, DR alpha |
| 228071_at | GIMAP7 | GTPase, IMAP family member 7 |
| 222496_s_at | FLJ20273 | NA |
| 206687_s_at | PTPN6 | protein tyrosine phosphatase, non-receptor type 6 |
| 210375_at | PTGER3 | prostaglandin E receptor 3 (subtype EP3) |
| 219440_at | RAI2 | retinoic acid induced 2 |
| 208450_at | LGALS2 | lectin, galactoside-binding, soluble, 2 (galectin 2) |
| 203665_at | HMOX1 | heme oxygenase (decycling) 1 |
| 227995_at | NA | NA |
| 227346_at | ZNFN1A1 | zinc finger protein, subfamily 1A, 1 (Ikaros) |
| 205159_at | CSF2RB | colony stimulating factor 2 receptor, beta, low-affinity |
| | | (granulocyte-macrophage) |
| 205559_s_at | PCSK5 | proprotein convertase subtilisin/kexin type 5 |
| 212886_at | CCDC69 | coiled-coil domain containing 69 |
| 1552612_at | CDC42SE2 | CDC42 small effector 2 |
| 205488_at | GZMA | granzyme A (granzyme 1, cytotoxic T-lymphocyte-associated |
| | | serine esterase 3) |
| 217767_at | C3 | complement component 3 |
| 208296_x_at | TNFAIP8 | tumor necrosis factor, alpha-induced protein 8 |
| 223484_at | C15orf48 | chromosome 15 open reading frame 48 |
| 204070_at | RARRES3 | retinoic acid receptor responder (tazarotene induced) 3 |
| 214719_at | LOC283537 | NA |
| 209687_at | CXCL12 | chemokine (C—X—C motif) ligand 12 (stromal cell-derived |
| | | factor 1) |
| 224773_at | NAV1 | neuron navigator 1 |
| 231882_at | NA | NA |
| 215223_s_at | SOD2 | superoxide dismutase 2, mitochondrial |
| 232617_at | CTSS | cathepsin S |
| 210554_s_at | CTBP2 | C-terminal binding protein 2 |
| 219528_s_at | BCL11B | B-cell CLL/lymphoma 11B (zinc finger protein) |
| 207861_at | CCL22 | chemokine (C-C motif) ligand 22 |
| 222592_s_at | ACSL5 | acyl-CoA synthetase long-chain family member 5 |
| 1554966_a_at | DOC1 | NA |
| 204204_at | SLC31A2 | solute carrier family 31 (copper transporters), member 2 |
| 219926_at | POPDC3 | popeye domain containing 3 |
| 204135_at | DOC1 | NA |
| 217995_at | SQRDL | sulfide quinone reductase-like (yeast) |
| 230233_at | RASGEF1B | RasGEF domain family, member 1B |
| 227265_at | FGL2 | fibrinogen-like 2 |
| 228372_at | C10orf128 | chromosome 10 open reading frame 128 |
| 204912_at | IL10RA | interleukin 10 receptor, alpha |
| 219454_at | EGFL6 | EGF-like-domain, multiple 6 |
| 206295_at | IL18 | interleukin 18 (interferon-gamma-inducing factor) |
| 226219_at | ARHGAP30 | Rho GTPase activating protein 30 |
| 218736_s_at | PALMD | palmdelphin |
| 223322_at | RASSF5 | Ras association (RalGDS/AF-6) domain family 5 |
| 209603_at | GATA3 | GATA binding protein 3 |
| 204687_at | DKFZP564O0823 | NA |
| 222895_s_at | BCL11B | B-cell CLL/lymphoma 11B (zinc finger protein) |
| 201010_s_at | TXNIP | thioredoxin interacting protein |
| 226818_at | DTX4 | deltex 4 homolog (Drosophila) |
| 208335_s_at | DARC | Duffy blood group, chemokine receptor |
| 213566_at | RNASE6 | ribonuclease, RNase A family, k6 |
| 205685_at | CD86 | CD86 antigen (CD28 antigen ligand 2, B7-2 antigen) |
| 201531_at | ZFP36 | zinc finger protein 36, C3H type, homolog (mouse) |
| 202391_at | BASP1 | brain abundant, membrane attached signal protein 1 |
| 201804_x_at | CKAP1 | cytoskeleton associated protein 1 |
| 206082_at | HCP5 | HLA complex P5 |
| 206204_at | GRB14 | growth factor receptor-bound protein 14 |
| 228563_at | GJA7 | gap junction protein, alpha 7, 45 kDa (connexin 45) |
| 219054_at | FLJ14054 | NA |
| 205844_at | VNN1 | vanin 1 |
| 203741_s_at | ADCY7 | adenylate cyclase 7 |
| 223280_x_at | MS4A6A | membrane-spanning 4-domains, subfamily A, member 6A |
| 205624_at | CPA3 | carboxypeptidase A3 (mast cell) |
| 209193_at | PIM1 | pim-1 oncogene |
| 210072_at | CCL19 | chemokine (C-C motif) ligand 19 |
| 226068_at | SYK | spleen tyrosine kinase |
| 216155_at | NAV1 | neuron navigator 1 |
| 205484_at | SIT1 | signaling threshold regulating transmembrane adaptor 1 |
| 228812_at | NA | NA |
| 219368_at | NAP1L2 | nucleosome assembly protein 1-like 2 |
| 206407_s_at | CCL13 | chemokine (C-C motif) ligand 13 |
| 203761_at | SLA | Src-like-adaptor |
| 236295_s_at | NOD3 | NA |
| 206099_at | PRKCH | protein kinase C, eta |
| 217143_s_at | TRD@ | T cell receptor delta locus |
| 218899_s_at | BAALC | brain and acute leukemia, cytoplasmic |
| 229391_s_at | RP1-93H18.5 | NA |
| 219093_at | FLJ20701 | NA |
| 219710_at | SH3TC2 | SH3 domain and tetratricopeptide repeats 2 |
| 206978_at | CCR2 | chemokine (C-C motif) receptor 2 |
| 204655_at | CCL5 | chemokine (C-C motif) ligand 5 |
| 211991_s_at | HLA-DPA1 | major histocompatibility complex, class II, DP alpha 1 |
| 223922_x_at | MS4A6A | membrane-spanning 4-domains, subfamily A, member 6A |
| 208983_s_at | PECAM1 | platelet/endothelial cell adhesion molecule (CD31 antigen) |
| 222108_at | AMIGO2 | adhesion molecule with Ig-like domain 2 |
| 1553102_a_at | CCDC69 | coiled-coil domain containing 69 |
| 221698_s_at | CLEC7A | C-type lectin domain family 7, member A |
| 206637_at | P2RY14 | purinergic receptor P2Y, G-protein coupled, 14 |
| 226459_at | PIK3AP1 | phosphoinositide-3-kinase adaptor protein 1 |
| 209613_s_at | ADH1B | alcohol dehydrogenase IB (class I), beta polypeptide |
| 225802_at | TOP1MT | topoisomerase (DNA) I, mitochondrial |
| 224859_at | CD276 | CD276 antigen |
| 209480_at | HLA-DQB1 | major histocompatibility complex, class II, DQ beta 1 |
| 219213_at | JAM2 | junctional adhesion molecule 2 |
| 208747_s_at | C1S | complement component 1, s subcomponent |
| 224356_x_at | MS4A6A | membrane-spanning 4-domains, subfamily A, member 6A |
| 226625_at | TGFBR3 | transforming growth factor, beta receptor III (betaglycan, |
| | | 300 kDa) |
| 1554240_a_at | ITGAL | c(“integrin, alpha L (antigen CD11A (p180), lymphocyte |
| | | function-associated antigen 1”, “alpha polypeptide)”) |
| 202948_at | IL1R1 | interleukin 1 receptor, type I |
| 219666_at | MS4A6A | membrane-spanning 4-domains, subfamily A, member 6A |
| 215193_x_at | HLA-DRB1 | major histocompatibility complex, class II, DR beta 1 |
| 232024_at | GIMAP2 | GTPase, IMAP family member 2 |
| 1559263_s_at | ZC3H12D | zinc finger CCCH-type containing 12D |
| 219737_s_at | PCDH9 | protocadherin 9 |
| 222838_at | SLAMF7 | SLAM family member 7 |
| 227983_at | MGC7036 | NA |
| 223809_at | RGS18 | regulator of G-protein signalling 18 |
| 213831_at | HLA-DQA1 | major histocompatibility complex, class II, DQ alpha 1 |
| 203416_at | CD53 | CD53 antigen |
| 226841_at | MPEG1 | NA |
| 228552_s_at | SSBP4 | single stranded DNA binding protein 4 |
| 231262_at | NA | NA |
| 206898_at | CDH19 | cadherin 19, type 2 |
| 210835_s_at | CTBP2 | C-terminal binding protein 2 |
| 227584_at | NAV1 | neuron navigator 1 |
| 223059_s_at | FAM107B | family with sequence similarity 107, member B |
| 221671_x_at | IGKC | immunoglobulin kappa constant |
| 205786_s_at | ITGAM | integrin, alpha M (complement component 3 receptor 3 |
| | | subunit) |
| 216194_s_at | CKAP1 | cytoskeleton associated protein 1 |
| 209312_x_at | HLA-DRB1 | major histocompatibility complex, class II, DR beta 1 |
| 235639_at | CDH19 | cadherin 19, type 2 |
| 233562_at | MGC16291 | NA |
| 232476_at | DDEF2 | development and differentiation enhancing factor 2 |
| 202510_s_at | TNFAIP2 | tumor necrosis factor, alpha-induced protein 2 |
| 222484_s_at | CXCL14 | chemokine (C—X—C motif) ligand 14 |
| 207277_at | CD209 | CD209 antigen |
| 204724_s_at | COL9A3 | collagen, type IX, alpha 3 |
| 239196_at | ANKRD22 | ankyrin repeat domain 22 |
| 209734_at | NCKAP1L | NCK-associated protein 1-like |
| 212977_at | CMKOR1 | chemokine orphan receptor 1 |
| 204670_x_at | HLA-DRB5 | major histocompatibility complex, class II, DR beta 5 |
| 208885_at | LCP1 | lymphocyte cytosolic protein 1 (L-plastin) |
| 209612_s_at | ADH1B | alcohol dehydrogenase IB (class I), beta polypeptide |
| 233955_x_at | CXXC5 | CXXC finger 5 |
| 228776_at | GJA7 | gap junction protein, alpha 7, 45 kDa (connexin 45) |
| 1553906_s_at | FGD2 | FYVE, RhoGEF and PH domain containing 2 |
| 221760_at | MAN1A1 | mannosidase, alpha, class 1A, member 1 |
| 223361_at | C6orf115 | chromosome 6 open reading frame 115 |
| 229390_at | RP1-93H18.5 | NA |
| 203915_at | CXCL9 | chemokine (C—X—C motif) ligand 9 |
| 223058_at | FAM107B | family with sequence similarity 107, member B |
| 219789_at | NPR3 | natriuretic peptide receptor C/guanylate cyclase C |
| | | (atrionatriuretic peptide receptor C) |
| 205285_s_at | FYB | FYN binding protein (FYB-120/130) |
| 203868_s_at | VCAM1 | vascular cell adhesion molecule 1 |
| 204236_at | FLI1 | Friend leukemia virus integration 1 |
| 224516_s_at | CXXC5 | CXXC finger 5 |
| 202368_s_at | TRAM2 | translocation associated membrane protein 2 |
| 224795_x_at | IGKC | immunoglobulin kappa constant |
| 235238_at | SHC4 | SHC (Src homology 2 domain containing) family, member 4 |
| 227791_at | SLC9A9 | solute carrier family 9 (sodium/hydrogen exchanger), member 9 |
| 207238_s_at | PTPRC | protein tyrosine phosphatase, receptor type, C |
| 204897_at | PTGER4 | prostaglandin E receptor 4 (subtype EP4) |
| 213975_s_at | LILRB1 | leukocyte immunoglobulin-like receptor, subfamily B (with |
| | | TM and ITIM domains), member 1 |
| 228964_at | PRDM1 | PR domain containing 1, with ZNF domain |
| 228362_s_at | RP1-93H18.5 | NA |
| 244061_at | ARHGAP15 | Rho GTPase activating protein 15 |
| 1553313_s_at | SLC5A3 | solute carrier family 5 (inositol transporters), member 3 |
| 212538_at | DOCK9 | dedicator of cytokinesis 9 |
| 226043_at | GPSM1 | G-protein signalling modulator 1 (AGS3-like, C. elegans) |
| 1405_i_at | CCL5 | chemokine (C-C motif) ligand 5 |
| 204222_s_at | GLIPR1 | GLI pathogenesis-related 1 (glioma) |
| 221087_s_at | APOL3 | apolipoprotein L, 3 |
| 203932_at | HLA-DMB | major histocompatibility complex, class II, DM beta |
| 225895_at | SYNPO2 | synaptopodin 2 |
| 221651_x_at | NA | NA |
| 231929_at | NA | NA |
| 229543_at | RP1-93H18.5 | NA |
| 211367_s_at | CASP1 | caspase 1, apoptosis-related cysteine peptidase (interleukin 1, |
| | | beta, convertase) |
| 210038_at | PRKCQ | protein kinase C, theta |
| 205403_at | IL1R2 | interleukin 1 receptor, type II |
| 220066_at | CARD15 | caspase recruitment domain family, member 15 |
| 1555812_a_at | ARHGDIB | Rho GDP dissociation inhibitor (GDI) beta |
| 208306_x_at | HLA-DRB4 | major histocompatibility complex, class II, DR beta 4 |
| 218854_at | SART2 | squamous cell carcinoma antigen recognized by T cells 2 |
| 203523_at | LSP1 | lymphocyte-specific protein 1 |
| 207992_s_at | AMPD3 | adenosine monophosphate deaminase (isoform E) |
| 228660_x_at | SEMA4F | sema domain, immunoglobulin domain (Ig), transmembrane |
| | | domain (TM) and short cytoplasmic domain, (semaphorin) 4F |
| 218170_at | ISOC1 | isochorismatase domain containing 1 |
| 1555759_a_at | CCL5 | chemokine (C-C motif) ligand 5 |
| 227253_at | HPS3 | Hermansky-Pudlak syndrome 3 |
| 211990_at | HLA-DPA1 | major histocompatibility complex, class II, DP alpha 1 |
| 212998_x_at | HLA-DQB1 | major histocompatibility complex, class II, DQ beta 1 |
| 204778_x_at | HOXB7 | homeobox B7 |
| 204834_at | FGL2 | fibrinogen-like 2 |
| 205039_s_at | ZNFN1A1 | zinc finger protein, subfamily 1A, 1 (Ikaros) |
| 232543_x_at | ARHGAP9 | Rho GTPase activating protein 9 |
| 209710_at | GATA2 | GATA binding protein 2 |
| 200612_s_at | AP2B1 | adaptor-related protein complex 2, beta 1 subunit |
| 201487_at | CTSC | cathepsin C |
| 201939_at | PLK2 | polo-like kinase 2 (Drosophila) |
| 203547_at | CD4 | CD4 antigen (p55) |
| 228376_at | GGTA1 | glycoprotein, alpha-galactosyltransferase 1 |
| 207574_s_at | GADD45B | growth arrest and DNA-damage-inducible, beta |
| 213560_at | GADD45B | growth arrest and DNA-damage-inducible, beta |
| 219525_at | FLJ10847 | NA |
| 204411_at | KIF21B | kinesin family member 21B |
| 200953_s_at | CCND2 | cyclin D2 |
| 201859_at | PRG1 | proteoglycan 1, secretory granule |
| 223044_at | SLC40A1 | solute carrier family 40 (iron-regulated transporter), member 1 |
| 213537_at | HLA-DPA1 | major histocompatibility complex, class II, DP alpha 1 |
| 216841_s_at | SOD2 | superoxide dismutase 2, mitochondrial |
| 205081_at | CRIP1 | cysteine-rich protein 1 (intestinal) |
| 226382_at | LOC283070 | NA |
| 219519_s_at | SIGLEC1 | sialic acid binding Ig-like lectin 1, sialoadhesin |
| 1558586_at | ZNF11B | zinc finger protein 11B |
| 217028_at | CXCR4 | chemokine (C—X—C motif) receptor 4 |
| 217478_s_at | HLA-DMA | major histocompatibility complex, class II, DM alpha |
| 204438_at | MRC1 | mannose receptor, C type 1 |
| 211366_x_at | CASP1 | caspase 1, apoptosis-related cysteine peptidase (interleukin 1, |
| | | beta, convertase) |
| 204249_s_at | LMO2 | LIM domain only 2 (rhombotin-like 1) |
| 221081_s_at | DENND2D | DENN/MADD domain containing 2D |
| 32128_at | CCL18 | chemokine (C-C motif) ligand 18 (pulmonary and activation- |
| | | regulated) |
| 220005_at | P2RY13 | purinergic receptor P2Y, G-protein coupled, 13 |
| 209924_at | CCL18 | chemokine (C-C motif) ligand 18 (pulmonary and activation- |
| | | regulated) |
| 242458_at | ANGPTL1 | angiopoietin-like 1 |
| 230391_at | NA | NA |
| 213475_s_at | ITGAL | c(“integrin, alpha L (antigen CD11A (p180), lymphocyte |
| | | function-associated antigen 1”, “alpha polypeptide)”) |
| 207458_at | C8orf51 | chromosome 8 open reading frame 51 |
| 235306_at | GIMAP8 | GTPase, IMAP family member 8 |
| 227780_s_at | NA | NA |
| 205841_at | JAK2 | Janus kinase 2 (a protein tyrosine kinase) |
| 202687_s_at | TNFSF10 | tumor necrosis factor (ligand) superfamily, member 10 |
| 212067_s_at | C1R | complement component 1, r subcomponent |
| 236908_at | ACPL2 | acid phosphatase-like 2 |
| 223827_at | TNFRSF19 | tumor necrosis factor receptor superfamily, member 19 |
| 213652_at | PCSK5 | proprotein convertase subtilisin/kexin type 5 |
| 219631_at | LRP12 | low density lipoprotein-related protein 12 |
| 1557116_at | NA | NA |
| 208981_at | PECAM1 | platelet/endothelial cell adhesion molecule (CD31 antigen) |
| 209685_s_at | PRKCB1 | protein kinase C, beta 1 |
| 238488_at | IPO11 | importin 11 |
| 1568736_s_at | DLGAP1 | discs, large (Drosophila) homolog-associated protein 1 |
| 238439_at | PRKAR2B | protein kinase, cAMP-dependent, regulatory, type II, beta |
| 205027_s_at | MAP3K8 | mitogen-activated protein kinase kinase kinase 8 |
| 211742_s_at | EVI2B | ecotropic viral integration site 2B |
| 202269_x_at | GBP1 | guanylate binding protein 1, interferon-inducible, 67 kDa |
| 204533_at | CXCL10 | chemokine (C—X—C motif) ligand 10 |
| 229163_at | CAMK2N1 | calcium/calmodulin-dependent protein kinase II inhibitor 1 |
| 1556579_s_at | MED12L | mediator of RNA polymerase II transcription, subunit 12 |
| | | homolog (yeast)-like |
| 201566_x_at | ID2 | inhibitor of DNA binding 2, dominant negative helix-loop-helix |
| | | protein |
| 201220_x_at | CTBP2 | C-terminal binding protein 2 |
| 214677_x_at | IGLJ3 | immunoglobulin lambda joining 3 |
| 235175_at | GBP4 | guanylate binding protein 4 |
| 232001_at | LOC439949 | NA |
| 228427_at | FBXO16 | F-box protein 16 |
| 214617_at | PRF1 | perforin 1 (pore forming protein) |
| 202369_s_at | TRAM2 | translocation associated membrane protein 2 |
| 202625_at | LYN | v-yes-1 Yamaguchi sarcoma viral related oncogene homolog |
| 213618_at | CENTD1 | centaurin, delta 1 |
| 209970_x_at | CASP1 | caspase 1, apoptosis-related cysteine peptidase (interleukin 1, |
| | | beta, convertase) |
| 218035_s_at | FLJ20273 | NA |
| 206715_at | TFEC | transcription factor EC |
| 1563473_at | PPP1R16B | protein phosphatase 1, regulatory (inhibitor) subunit 16B |
| 204118_at | CD48 | CD48 antigen (B-cell membrane protein) |
| 201137_s_at | HLA-DPB1 | major histocompatibility complex, class II, DP beta 1 |
| 230538_at | SHC4 | SHC (Src homology 2 domain containing) family, member 4 |
| 1568822_at | GTPBP5 | GTP binding protein 5 (putative) |
| 229625_at | GBP5 | guanylate binding protein 5 |
| 212233_at | MAP1B | microtubule-associated protein 1B |
| 209202_s_at | EXTL3 | exostoses (multiple)-like 3 |
| 209083_at | CORO1A | coronin, actin binding protein, 1A |
| 205226_at | PDGFRL | platelet-derived growth factor receptor-like |
| 227640_s_at | RP9 | retinitis pigmentosa 9 (autosomal dominant) |
| 223168_at | RHOU | ras homolog gene family, member U |
| 1553132_a_at | MTAC2D1 | membrane targeting (tandem) C2 domain containing 1 |
| 214038_at | CCL8 | chemokine (C-C motif) ligand 8 |
| 219505_at | CECR1 | cat eye syndrome chromosome region, candidate 1 |
| 214669_x_at | IGKC | immunoglobulin kappa constant |
| 233123_at | SLC40A1 | solute carrier family 40 (iron-regulated transporter), member 1 |
| 209195_s_at | ADCY6 | adenylate cyclase 6 |
| 204846_at | CP | ceruloplasmin (ferroxidase) |
| 204642_at | EDG1 | endothelial differentiation, sphingolipid G-protein-coupled |
| | | receptor, 1 |
| 239744_at | RGS3 | regulator of G-protein signalling 3 |
| 206545_at | CD28 | CD28 antigen (Tp44) |
| 228339_at | NA | NA |
| 218739_at | ABHD5 | abhydrolase domain containing 5 |
| 224358_s_at | MS4A7 | membrane-spanning 4-domains, subfamily A, member 7 |
| 1559425_at | PRKCH | protein kinase C, eta |
| 231577_s_at | GBP1 | guanylate binding protein 1, interferon-inducible, 67 kDa |
| 231032_at | LOC286071 | NA |
| 207655_s_at | BLNK | B-cell linker |
| 242546_at | NA | NA |
| 211066_x_at | PCDHGC3 | protocadherin gamma subfamily C, 3 |
| 216714_at | CCL13 | chemokine (C-C motif) ligand 13 |
| 1562031_at | JAK2 | Janus kinase 2 (a protein tyrosine kinase) |
| 212763_at | CAMSAP1L1 | calmodulin regulated spectrin-associated protein 1-like 1 |
| 205440_s_at | NPY1R | neuropeptide Y receptor Y1 |
| 227458_at | CD274 | CD274 antigen |
| 226303_at | PGM5 | phosphoglucomutase 5 |
| 204613_at | PLCG2 | phospholipase C, gamma 2 (phosphatidylinositol-specific) |
| 202688_at | TNFSF10 | tumor necrosis factor (ligand) superfamily, member 10 |
| 221477_s_at | SOD2 | superoxide dismutase 2, mitochondrial |
| 201236_s_at | BTG2 | BTG family, member 2 |
| 205569_at | LAMP3 | lysosomal-associated membrane protein 3 |
| 215121_x_at | IGLC1 | immunoglobulin lambda constant 1 (Mcg marker) |
| 202255_s_at | SIPA1L1 | signal-induced proliferation-associated 1 like 1 |
| 215051_x_at | AIF1 | allograft inflammatory factor 1 |
| 209138_x_at | IGLC2 | immunoglobulin lambda constant 2 (Kern-Oz-marker) |
| 232311_at | B2M | beta-2-microglobulin |
| 1555756_a_at | CLEC7A | C-type lectin domain family 7, member A |
| 221756_at | MGC17330 | NA |
| 238544_at | IGF1R | insulin-like growth factor 1 receptor |
| 204512_at | HIVEP1 | human immunodeficiency virus type I enhancer binding protein 1 |
| 230728_at | FKBP14 | FK506 binding protein 14, 22 kDa |
| 201720_s_at | LAPTM5 | lysosomal associated multispanning membrane protein 5 |
| 223395_at | ABI3BP | ABI gene family, member 3 (NESH) binding protein |
| 200905_x_at | HLA-E | major histocompatibility complex, class I, E |
| 202207_at | ARL4C | ADP-ribosylation factor-like 4C |
| 207076_s_at | ASS | argininosuccinate synthetase |
| 211368_s_at | CASP1 | caspase 1, apoptosis-related cysteine peptidase (interleukin 1, |
| | | beta, convertase) |
| 204628_s_at | ITGB3 | integrin, beta 3 (platelet glycoprotein IIIa, antigen CD61) |
| 207540_s_at | SYK | spleen tyrosine kinase |
| 213603_s_at | RAC2 | ras-related C3 botulinum toxin substrate 2 (rho family, small |
| | | GTP binding protein Rac2) |
| 1557222_at | NA | NA |
| 206804_at | CD3G | CD3g antigen, gamma polypeptide (TiT3 complex) |
| 209542_x_at | IGF1 | insulin-like growth factor 1 (somatomedin C) |
| 228858_at | NA | NA |
| 207843_x_at | CYB5A | cytochrome b5 type A (microsomal) |
| 223924_at | TTC25 | tetratricopeptide repeat domain 25 |
| 1552497_a_at | SLAMF6 | SLAM family member 6 |
| 241701_at | ARHGAP21 | Rho GTPase activating protein 21 |
| 213819_s_at | FLOT1 | flotillin 1 |
| 213095_x_at | AIF1 | allograft inflammatory factor 1 |
| 228153_at | IBRDC2 | IBR domain containing 2 |
| 213007_at | KIAA1794 | KIAA1794 |
| 217525_at | OLFML1 | olfactomedin-like 1 |
| 204220_at | GMFG | glia maturation factor, gamma |
| 203508_at | TNFRSF1B | tumor necrosis factor receptor superfamily, member 1B |
| 217629_at | NA | NA |
| 226659_at | DEF6 | differentially expressed in FDCP 6 homolog (mouse) |
| 200904_at | HLA-E | major histocompatibility complex, class I, E |
| 206571_s_at | MAP4K4 | mitogen-activated protein kinase kinase kinase kinase 4 |
| 232746_at | CMKOR1 | chemokine orphan receptor 1 |
| 1563461_at | NA | NA |
| 204233_s_at | CHKA | choline kinase alpha |
| 226865_at | NA | NA |
| 227361_at | HS3ST3B1 | heparan sulfate (glucosamine) 3-O-sulfotransferase 3B1 |
| 204923_at | CXorf9 | chromosome X open reading frame 9 |
| 204774_at | EVI2A | ecotropic viral integration site 2A |
| 202270_at | GBP1 | guanylate binding protein 1, interferon-inducible, 67 kDa |
| 243099_at | NFAM1 | NFAT activating protein with ITAM motif 1 |
| 242874_at | NA | NA |
| 229127_at | ATP5J | ATP synthase, H+ transporting, mitochondrial F0 complex, |
| | | subunit F6 |
| 224771_at | NAV1 | neuron navigator 1 |
| 215379_x_at | IGLC2 | immunoglobulin lambda constant 2 (Kern-Oz-marker) |
| 222142_at | CYLD | cylindromatosis (turban tumor syndrome) |
| 229367_s_at | GIMAP6 | GTPase, IMAP family member 6 |
| 212713_at | MFAP4 | microfibrillar-associated protein 4 |
| 214023_x_at | TUBB2B | tubulin, beta 2B |
| 203413_at | NELL2 | NEL-like 2 (chicken) |
| 236583_at | NA | NA |
| 212657_s_at | IL1RN | interleukin 1 receptor antagonist |
| 227231_at | KIAA1211 | NA |
| 244023_at | SYK | spleen tyrosine kinase |
| 206134_at | ADAMDEC1 | ADAM-like, decysin 1 |
| 204894_s_at | AOC3 | amine oxidase, copper containing 3 (vascular adhesion protein |
| | | 1) |
| 204502_at | SAMHD1 | SAM domain and HD domain 1 |
| 218002_s_at | CXCL14 | chemokine (C—X—C motif) ligand 14 |
| 205421_at | SLC22A3 | solute carrier family 22 (extraneuronal monoamine transporter), |
| | | member 3 |
| 215561_s_at | IL1R1 | interleukin 1 receptor, type I |
| 217138_x_at | IGLV3-25 | immunoglobulin lambda variable 3-25 |
| 1556185_a_at | NA | NA |
| 219681_s_at | RAB11FIP1 | RAB11 family interacting protein 1 (class I) |
| 205251_at | PER2 | period homolog 2 (Drosophila) |
| 224896_s_at | TTL | tubulin tyrosine ligase |
| 209899_s_at | SIAHBP1 | NA |
| 201721_s_at | LAPTM5 | lysosomal associated multispanning membrane protein 5 |
| 241671_x_at | FLJ22536 | NA |
| 218499_at | RP6- | NA |
| | 213H19.1 | |
| 235804_at | NA | NA |
| 207677_s_at | NCF4 | neutrophil cytosolic factor 4, 40 kDa |
| 227609_at | EPSTI1 | epithelial stromal interaction 1 (breast) |
| 227035_x_at | LOC441212 | NA |
| 206385_s_at | ANK3 | ankyrin 3, node of Ranvier (ankyrin G) |
| 1565602_at | PCDH9 | protocadherin 9 |
| 228908_s_at | C21orf86 | chromosome 21 open reading frame 86 |
| 223952_x_at | DHRS9 | dehydrogenase/reductase (SDR family) member 9 |
| 38149_at | ARHGAP25 | Rho GTPase activating protein 25 |
| 235688_s_at | TRAF4 | TNF receptor-associated factor 4 |
| 214181_x_at | LST1 | leukocyte specific transcript 1 |
| 222725_s_at | PALMD | palmdelphin |
| 1555852_at | TAP1 | transporter 1, ATP-binding cassette, sub-family B (MDR/TAP) |
| 210319_x_at | MSX2 | msh homeobox homolog 2 (Drosophila) |
| 220485_s_at | SIRPG | signal-regulatory protein gamma |

TABLE 5
Primers and probes used for Q-PCR in standard 96 well plates

| Gene | | | |
|---|---|---|---|
| name | Forward primers | Reverse primers | TaqMan Probes |
| IL7R | TCC-TAC-CAG-CCT-TTG-CCT-CTT | CCA-TAC-TTG-GCT-TTT-CTG-AAG-CA | FAM-CTT-CAA-TGT-GGT-TTC- |
| | | | CAT-GGG-MGB |
| CD3D | GTT-GAG-GAA-TGA-CCA-GGT-CTA- | AAG-GTG-GCT-GTA-CTG-AGC-ATC-A | FAM-CCT-CCG-AGA-TCG-AG- |
| | TCA-G | | MGB |
| CD52 | TCA-GCC-TCC-TGG-TTA-TGG-TAC- | TGG-TTT-GGC-TGG-TGT-CGT-T | FAM-CAA-ACT-GGA-CTC-TCA- |
| | AG | | GGA-C-MGB |
| UBD | TGA-GGA-GCT-GCC-CTT-GTT-TC | CTG-GAG-GAG-GTG-CCT-CTT-TG | FAM-CAT-CAC-CTG-ACT-CCA- |
| | | | CA-MGB |
| GPR171 | GAA-GTC-ATA-ACT-GAT-TGC-TCA- | GAC-ACA-GCC-AGG-AGC-AGT-GTA-G | FAM-TCA-CTC-TTC-AAA-GCC- |
| | ACC-A | | AAA-GA-MGB |
| GMZK | GTC-TGT-GCA-GGA-GAT-GGC-AA | GGG-CCC-CCT-GAG-TCA-CC | FAM_TTA-CAG-GAA-TCC-TGG- |
| | | | C_MGB |
| PRKCQ | ATC-CAT-GGT-CAA-GAG-GAC-AAG- | GAC-AAG-ATA-ACA-AGC-GAA-GGT-GTC | FAM_CAA-ACC-TTC-CCT-CTC- |
| | TGG-A | | ACT_MGB |
| STAT4 | GTC-CCA-CAA-CAA-TTG-AAA-CTG- | GTG-CGT-CAG-AGT-TTA-TCC-TGT-CAT | FAM_TGA-AGT-CTC-CTT-ATT- |
| | C | | CTG-CT_MGB |
| TRDV2 | GGT-CGA-GAA-AAG-CTT-TGA-AAC- | GGA-ACC-CAA-TCA-CTG-ACA-GGT-T | FAM_ACG-AAC-CTA-AAC-TTT- |
| | AG | | C_MGB |
| TRAT1 | AAG-CCA-CCC-CAT-CTG-CAC | CTG-TGA-TCA-AGT-GAG-GCG-TAG | FAM_CAA-CCA-ATG-AAA-CAC- |
| | | | AGA-TG_MGB |
| TRBV19 | GAG-GCC-TGG-GGT-AGA-GCA-G | AGA-GGA-TGG-TGG-CAG-ACA-GG | FAM_TGT-CCT-ACC-AGC-AAG- |
| | | | GG_MGB |
| CD69 | AAT-CAT-AGC-TCT-CAT-TGC-CTT- | TGT-CTG-ATG-GCA-TTG-AGA-ATG-TG | FAM_CCA-ATA-CAA-TTG-TCC- |
| | ATC-AG | | AGG-CC_MGB |
| INDO1 | CCA-GCA-GAC-TGC-TGG-TGG-A | GGC-ATA-TAT-CTT-CTC-ATG-TCC-TGG-A | FAM_ACA-TGC-TGC-TCA-GTT- |
| | | | C_MGB |
| H3F3A | CAG-CTA-TCG-GTG-CTT-TGC-AG | GAT-AGC-ACA-CAG-GTT-GGT-GTC-TTC | FAM_CAA-GTG-AGG-CCT-ATC- |
| | | | T_MGB |
| CD45R | GAG-ATG-CCT-GAT-GGT-TCA-AGT- | TTC-TTC-AAA-CTG-ATT-GTA-TTC-CAC-C | FAM_CAG-TAC-ATC-TTG-ATC- |
| | AGA-G | | CAT-C_MGB |
| CD45RO | GGC-AAA-GCC-CAA-CAC-CTT-C | CAG-AAG-GGC-TCA-GAG-TGG-TTG-T | FAM_TGC-CTA-CCT-TAA-TGC- |
| | | | C_MGB |
| FoxP3 | CAC CTG GCT GGG AAA ATG G | GGAGCC CTT GTC GGA TGA T | FAM_CAA GGC TTC ATC TGT |
| | | | G_MGB |
| CD20 | CAT-ACA-ATC-TCT-GTT-CTT-GGG- | TCA-TTC-TCA-ACG-ATG-CCA-GCT-A | FAM_TCA-GTG-ATG-CTG-ATC- |
| | CAT-T | | TT_MGB |
| TLR9 | CGC-CAG-ACC-CTC-TGG-AGA-A | GCC-TGC-ACC-AGG-AGA-GAC-A | FAM_TTC-TGC-CGC-AGC-G_MGB |
| ITGB3 | AAG-TCC-ATC-CTG-TATG-TGG- | GAG-CAG-GAC-CAC-CAG-GAT-GT | FAM_AGT-GTC-CCA-AGG-GC_MGB |
| | TAG-AAG-AG | | |
| TLR3 | GGA-AAG-GCT-AGC-AGT-CAT-CCA | GTG-GAG-GAT-GCA-CAC-AGC-AT | FAM_ATG-AGA-CAG-ACT-TTG- |
| | | | CC_MGB |
| ThR4 | CCA-GAA-CTG-CAG-GTG-CTG-G | GGT-GGC-TTA-GGC-TCT-GAT-ATG-C | FAM_CCA-GGT-GTG-AAA-TCC- |
| | | | AGA-CA_MGB |

| | |
|---|---|
| TLR7 | Assay-on-demand Hs00152971_m1 (Applied Biosystens) |
| TLR10 | Assay-on-demand Hs01675179_m1 (Applied Biosystems) |
| variant 1 | |
| IRF1 | Assay-on-demand Hs00971965_m1 (Applied Biosystems) |

TABLE 6
Gene included into the TaqMan ® Immune Profiling Array

| | | | |
|---|---|---|---|
| | Gene | Target | |
| Assay ID | Symbol | Exons | NCBI Gene Reference |

| | | | |
|---|---|---|---|
| Hs00174092_m1 | IL1A | 6 | NM_000575, M15329, X02531, X02851, X56086, CR457414, BC035169, BC013142, |
| | | | BT007014 |
| Hs00174097_m1 | IL1B | 5 | NM_000576, K02770, M15330, M54933, X02532, X56087, AF043335, BC008678, |
| | | | CR407679, BT007213 |
| Hs00174114_m1 | 1L2 | 2 | NM_000586, U25676,,V00564,,X01586,,S82692, S77835, S77834, AF228636, |
| | | | BC066255, BC066256, BC06 [symbol] |
| | | | C066254, AY523040, BC070338 A14844 |
| Hs00174117_m1 | 1L3 | 2 | NM_000588, M17115, M14743, M20137, BC066274, BC066276, BC066272, BC066273, |
| | | | BC069472 |
| Hs00174122_m1 | 1L4 | 3 | NM_172348, NM_000589, M13982, X81851, AF043336, BC066277, BC067514, BC067515, |
| | | | BC070123, AB1 [symbol] |
| Hs00174200_m1 | ILS | 2 | NM_000879, X12705, X04688, BC066282 |
| Hs00174131_m1 | 1L6 | 3 | NM_000600, X04403, M14584, M18403, M29150, X04430, X04602, M54894, S56892, |
| | | | CR450296, BT019748 |
| | | | 9749, A09363, BC015511 |
| Hs00174202_m1 | 1L7 | 2 | NM_000880, J04156, BC047698 |
| Hs00174103_m1 | 1L8 | 1 | NM_000584, M17017, M26383, Z11686, Y00787, CR542151, AK131067, BC013615, |
| | | | BT007067 |
| Hs00174125_m1 | 1L9 | 3 | NM_000590, M30134, X17543, S63356, BC066284, BC066283, BC066285, BC066286 |
| Hs99999901_s1 | 18S | 1 | X03205 |
| Hs00174086_m1 | IL10 | 3 | NM_000572, M57627, AF043333, AY029171, CR541993 |
| Hs00168405_m1 | IL12A | 2 | NM_000882, M65271, M65291, AF101062, AF180562, BG702253 |
| Hs00233688_m1 | IL12B | 3 | NM_002187, M65272, M65290, AF180563, BC067501, BC067498, BC067499, BC067500, |
| | | | BC067502, BC07 [symbol] |
| Hs00174379_m1 | IL13 | 1 | NM_002188, L06801, X69079, BC096141, BC096139 |
| Hs00174106_m1 | 1L15 | 1 | NM_000585, U14407 |
| Hs00174383_m1 | IL17 | 2 | NM_002190, Z58820, U32659, BC066251 ,BC066252, BC066253, BC067504, BC067503, |
| | | | BC067505 |
| Hs00155517_m1 | IL18 | 4 | NM_001562, D49950, U90434, AF077611, AY266351, CR541973, CR542001, AY044641, |
| | | | BCO15863, BC00 [symbol] |
| Hs00234142_m1 | CCL3 | 1 | NM_002983, M25315, D63785, M23452, D00044, X03754, BC071834 |
| Hs00171149_m1 | CCL19 | 1 | NM_006274, AB000887, U77180, U88321, CR456868, BC027968 |
| Hs00234140_m1 | CCL2 | 1 | NM_002982, M24545, M28226, X14768, S69738, S71513, AV733621, BC009716, |
| | | | BT007329 |
| Hs00174575_m1 | CCL5 | 2 | NM_002985, M21121, AF043341, BC008600, AF266753 |
| Hs00174150_m1 | CCR2 | 1 | NM_000647, U03882, BC074751 |
| Hs99999919_m1 | CCR4 | N/A | NM_005508, BC069139, BC071751, BC074935 |
| Hs00152917_m1 | CCR5 | 2 | NM_000579, U54994, BC038398 |
| Hs00171054_m1 | CCR7 | 1 | NM_001838, L31581, L08176, BC035343, AK127810 |
| Hs00171041_m1 | CXCR3 | 1 | NM_001504, X95876 |
| Hs00171042_m1 | CXCLI0 | 1 | NM_001565, X02530, BC010954 |
| Hs00171138_m1 | CXCL11 | 1 | NM_005409, Y15220, U59286, AF030514, AF002985, U66096, BC005292, AF352781, |
| | | | BC012532, BT00678 [symbol] |
| Hs00174164_m1 | CSF1 | 4 | NM_172210, NM_172211, NM_172212, NM_000757, U22386, M37435, M76453, M64592, |
| | | | M27087, X05825, [symbol] |
| Hs00171266_m1 | CSF2 | 3 | NM_000758, M10663, M11220, AY720441, AF510855 |
| Hs00357085_g1 | CSF3 | 1 | NM_172219, NM_000759, M17706, X03438, X03655, CR541891 |
| Hs00234174_m1 | STAT3 | N/A | NM_139276, AJ012463, L29277, BC014482 |
| Hs00174517_m1 | NFKB2 | 6 | NM_002502, U09609, X61498, X61499, S76638, BC002844, BT009769 |
| Hs00395088_m1 | IKBKB | 8 | NM_001556, AF029684, AF031416, AF080158, AB209090, BX648165 |
| Hs00167894_m1 | CD3E | 1 | NM_000733, X03884, B1910359 |
| Hs00181217_m1 | CD4 | 2 | NM_000616, M12807, BT019791, BT019811, BC025782 |
| Hs00233520_m1 | CD8A | 5 | NM_171827, NM_001768, M12824, M12628, AY039664, AK124156, BC025715, AK097942 |
| Hs00174333_m1 | CD19 | 4 | NM_001770, M21097, XI3312, BC006338, AK130657, BC052294 |
| Hs00166229_m1 | IL2RA | 1 | NM_000417, X01057, K03122, AK223313 |
| Hs00174796_m1 | CD28 | 2 | NM_006139, J02988, BC093698 |
| Hs00233552_m1 | CD38 | 1 | NM_001775, D84276, M34461, BC007964, CD688069 |
| Hs00374176_m1 | CD40 | 1 | NM_152854, NM_001250, AJ300189, BC064518, BC012419, BM761221, AK222896 |
| Hs00365634_g1 | PTPRC | 1 | NM_080921, NM_080922, NM_080923, NM_002838, Y00062, BC017863, BC014239 |
| Hs00154355_m1 | CD68 | 6 | NM_001251, S57235, BC015557, BT009923, AK222492, AK222514 |
| Hs00175478_m1 | CD80 | 2 | NM_005191, M27533, AY197777, AY197778, AY081815, BC042665 |
| Hs00199349_m1 | CD86 | 2 | NM_175862, NM_006889, L25259, U04343, CR541844, BC040261 |
| Hs00175480_m1 | CTLA4 | 1 | NM_005214, L15006, BC069566, BC070162, BC074842, BC074893, AY209009, |
| | | | AF414120, AF486806 |
| Hs00163934_m1 | CD40LG | 4 | NM_000074, L07414, X67878, Z15017, X68550, BC071754, BC074950 |
| Hs00219575_m1 | HLA-DRA | 1 | NM_019111, J00194, M60334, M35979, K01171, M60333, CR457013, BC071659, |
| | | | BC032350 |
| Hs99999917_m1 | HLA-DRB1 | N/A | NM_002124,M11161, M33600, X03069, DQ002917 |
| Hs00203436_m1 | TBX21 | 1 | NM_013351, AF093098, AF241243, BC039739, AK223634 |
| Hs00188346_m1 | TNFRSF18 | 2 | NM_148901, NM_148902, NM_004195, AF117297, AF125304, AF241229, BT019531, |
| | | | BT019532, AY358877 |
| Hs00359999_m1 | ICOS | 1 | NM_012092, AB023135, AJ277832, BC028006, BC028210 |
| Hs00167248_m1 | NOS2A | 11 | NM_153292, NM_000625, AB022318, U05810, D26525, L24553, X73029, U20141, |
| | | | U31511, S75615 |
| Hs00153350_m1 | BCL2 | 1 | NM_000633, M13994, M14745, X06487, BC027258 |
| Hs00169141_m1 | BCL2L1 | 1 | NM 001191, Z23116 |
| Hs00180269_m1 | BAX | 3 | NM_138761, NM_138763, NM_138765, NM_004324, L22473, L22474, U19599, AF247393, |
| | | | AF250190, AJ586909, AJ586910, BM706954, BC014175, AJ417988 |
| Hs00164932_m1 | ICAM1 | 2 | NM_000201, M24283, J03132, X06990, AF340038, AF340039, BC015969, AK130659, BT006854 |
| Hs00174583_m1 | SELP | 1 | NM_003005, M25322, BC068533 |
| Hs00174057_m1 | SELE | 4 | NM_000450, M24736, M30640 |
| Hs00157965_m1 | HM0X1 | 2 | NM_002133, M23041, X06985, CR456505, CR541945, CR541968, BT019785, BC001491 |
| Hs00153133_m1 | PTGS2 | 5 | NM_000963, M90100, L15326, AJ634912, AY462100, BC013734, AY151286 |
| Hs00166915_m1 | REN | 1 | NM_000537, CR536498, BC047752, BC033474 |
| Hs00189742_m1 | LRP2 | 17 | NM_004525, U33837, AY265357, AY265358 |
| Hs00411908_m1 | MYH6 | 23 | NM_002471, D00943 |
| Hs00192564_m1 | RPL3L | 3 | NM_005061, U65581, BC050413 |
| Hs00167927_m1 | CYP1A2 | 2 | NM_000761, M55053, Z00036, AF182274, BC067425, BC067427, BC067424, BC067429, |
| | | | BC067426, BC067428 |
| Hs00167982_m1 | CYP7A1 | 3 | NM_000780, X56088, M93133 |
| Hs00174143_m1 | IFNG | 1 | NM_000619, X13274, V00543, X87308, BC070256, AY255837, AY255839, AY044154, |
| | | | AF506749 |
| Hs00169473_m1 | PRF1 | 1 | NM_005041, M28393, X13224, BC063043, BC047695 |

[symbol]

indicates data missing or illegible when filed

TABLE 7A
Geomean ratio between responder and non-responder groups

| Gene | Ratio | LL | UL |
|---|---|---|---|
| CCL5 | 7.83 | 3.09 | 19.84 |
| FASLG | 7.57 | 2.77 | 20.70 |
| GNLY | 8.82 | 3.61 | 21.56 |
| GZMB | 12.15 | 4.38 | 33.71 |
| PRF1 | 11.07 | 4.85 | 25.28 |
| IFNG | 21.19 | 5.36 | 83.86 |
| ICOS | 13.44 | 3.44 | 52.51 |
| TBX21 | 13.93 | 3.63 | 53.41 |
| CD8A | 11.60 | 4.22 | 31.90 |
| CD3E | 12.78 | 3.55 | 46.04 |
| CXCL11 | 4.71 | 1.44 | 15.35 |
| CXCL10 | 7.39 | 2.56 | 21.39 |
| CXCR3 | 16.96 | 3.10 | 92.90 |
| CD20 | 9.46 | 1.35 | 66.42 |
| FOXP3 | 5.57 | 2.58 | 12.04 |
| INDO | 13.62 | 2.89 | 64.20 |
| CD45Ro | 4.02 | 1.84 | 8.79 |
| CD45R | 4.92 | 1.90 | 12.77 |
| CD69 | 5.84 | 2.00 | 17.06 |
| TRBV19 | 11.15 | 3.56 | 34.94 |
| TRAT1 | 9.67 | 2.77 | 33.80 |
| TRDV2 | 7.05 | 2.79 | 17.82 |
| STAT4 | 3.41 | 1.75 | 6.65 |
| PRKCQ | 7.41 | 3.24 | 16.94 |
| GMZK | 9.20 | 2.60 | 32.49 |
| GPR171 | 5.25 | 2.47 | 11.17 |
| UBD | 14.68 | 3.95 | 54.65 |
| CD52 | 6.17 | 1.96 | 19.41 |
| CD3D | 10.03 | 3.07 | 32.77 |
| IL7R | 6.49 | 2.08 | 20.26 |
| IRF1 | 5.54 | 2.83 | 10.83 |
| TLR7 | 3.44 | 1.50 | 7.89 |

TABLE 8 (BELOW)
Correlation matrix between 30 genes

| Variable | Ig_IL7R | Ig_CD3D | Ig_CD52 | Ig_UBD | Ig_GPR171 | Ig_GMZK | Ig_PRKCQ | Ig_STAT4 |
|---|---|---|---|---|---|---|---|---|
| Ig_IL7R | 1.00 | 0.90 | 0.91 | 0.79 | 0.80 | 0.80 | 0.83 | 0.84 |
| Ig_CD3D | 0.90 | 1.00 | 0.94 | 0.93 | 0.89 | 0.93 | 0.88 | 0.88 |
| Ig_CD52 | 0.91 | 0.94 | 1.00 | 0.80 | 0.83 | 0.82 | 0.84 | 0.83 |
| Ig_UBD | 0.79 | 0.93 | 0.80 | 1.00 | 0.82 | 0.91 | 0.80 | 0.82 |
| Ig_GPR17 | 0.80 | 0.89 | 0.83 | 0.82 | 1.00 | 0.81 | 0.94 | 0.92 |
| Ig_GMZK | 0.80 | 0.93 | 0.82 | 0.91 | 0.81 | 1.00 | 0.83 | 0.85 |
| Ig_PRKC [symbol] | 0.83 | 0.88 | 0.84 | 0.80 | 0.94 | 0.83 | 1.00 | 0.93 |
| Ig_STAT4 | 0.84 | 0.88 | 0.83 | 0.82 | 0.92 | 0.85 | 0.93 | 1.00 |
| Ig_TRDV2 | 0.88 | 0.96 | 0.91 | 0.86 | 0.94 | 0.87 | 0.93 | 0.89 |
| Ig_TRAT1 | 0.92 | 0.96 | 0.89 | 0.89 | 0.85 | 0.95 | 0.89 | 0.91 |
| Ig_TRBV1 | 0.92 | 0.99 | 0.95 | 0.91 | 0.90 | 0.92 | 0.91 | 0.89 |
| Ig_CD69 | 0.85 | 0.92 | 0.92 | 0.80 | 0.80 | 0.88 | 0.87 | 0.87 |
| Ig_CD45R | 0.90 | 0.91 | 0.88 | 0.80 | 0.83 | 0.88 | 0.89 | 0.90 |
| Ig_CD45R | 0.84 | 0.91 | 0.83 | 0.83 | 0.86 | 0.90 | 0.89 | 0.88 |
| Ig_INDO | 0.76 | 0.89 | 0.79 | 0.91 | 0.81 | 0.87 | 0.83 | 0.83 |
| Ig_FOXP3 | 0.78 | 0.80 | 0.75 | 0.74 | 0.83 | 0.78 | 0.91 | 0.84 |
| Ig_CD20 | 0.87 | 0.82 | 0.88 | 0.69 | 0.67 | 0.74 | 0.74 | 0.74 |
| Ig_CXCR3 | 0.84 | 0.89 | 0.82 | 0.85 | 0.74 | 0.86 | 0.79 | 0.78 |
| Ig_CXCL1 [symbol] | 0.65 | 0.84 | 0.68 | 0.92 | 0.76 | 0.83 | 0.72 | 0.73 |
| Ig_CXCL1 [symbol] | 0.54 | 0.74 | 0.60 | 0.81 | 0.75 | 0.74 | 0.71 | 0.77 |
| Ig_CD3E | 0.86 | 0.94 | 0.92 | 0.81 | 0.85 | 0.87 | 0.88 | 0.83 |
| Ig_CD8A | 0.77 | 0.93 | 0.82 | 0.89 | 0.88 | 0.91 | 0.91 | 0.87 |
| Ig_TBX21 | 0.91 | 0.97 | 0.93 | 0.90 | 0.83 | 0.87 | 0.81 | 0.81 |
| Ig_ICOS | 0.85 | 0.94 | 0.87 | 0.89 | 0.78 | 0.87 | 0.78 | 0.79 |
| Ig_IFNG | 0.75 | 0.91 | 0.78 | 0.92 | 0.74 | 0.92 | 0.75 | 0.78 |
| Ig_PRF1 | 0.76 | 0.92 | 0.82 | 0.89 | 0.88 | 0.91 | 0.91 | 0.87 |
| Ig_GZMB | 0.80 | 0.92 | 0.89 | 0.86 | 0.90 | 0.84 | 0.88 | 0.87 |
| Ig_GNLY | 0.74 | 0.87 | 0.84 | 0.77 | 0.85 | 0.83 | 0.88 | 0.82 |
| Ig_FASLG | 0.68 | 0.87 | 0.78 | 0.85 | 0.82 | 0.85 | 0.81 | 0.81 |
| Ig_CCL5 | 0.77 | 0.93 | 0.83 | 0.91 | 0.83 | 0.92 | 0.81 | 0.82 |

| Variable | Ig_TRDV2 | Ig_TRAT1 | Ig_TRBV19 | Ig_CD69 | Ig_CD45R | Ig_CD45RO | Ig_INDO | Ig_FOXP3 |
|---|---|---|---|---|---|---|---|---|
| Ig_IL7R | 0.88 | 0.92 | 0.92 | 0.85 | 0.90 | 0.84 | 0.76 | 0.78 |
| Ig_CD3D | 0.96 | 0.96 | 0.99 | 0.92 | 0.91 | 0.91 | 0.89 | 0.80 |
| Ig_CD52 | 0.91 | 0.89 | 0.95 | 0.92 | 0.88 | 0.83 | 0.79 | 0.75 |
| Ig_UBD | 0.86 | 0.89 | 0.91 | 0.80 | 0.80 | 0.83 | 0.91 | 0.74 |
| Ig_GPR17 | 0.94 | 0.85 | 0.90 | 0.80 | 0.83 | 0.86 | 0.81 | 0.83 |
| Ig_GMZK | 0.87 | 0.95 | 0.92 | 0.88 | 0.88 | 0.90 | 0.87 | 0.78 |
| Ig_PRKC [symbol] | 0.93 | 0.89 | 0.91 | 0.87 | 0.89 | 0.89 | 0.83 | 0.91 |
| Ig_STAT4 | 0.89 | 0.91 | 0.89 | 0.87 | 0.90 | 0.88 | 0.83 | 0.84 |
| Ig_TRDV2 | 1.00 | 0.92 | 0.97 | 0.88 | 0.91 | 0.91 | 0.83 | 0.85 |
| Ig_TRAT1 | 0.92 | 1.00 | 0.96 | 0.92 | 0.95 | 0.93 | 0.87 | 0.82 |
| Ig_TRBV1 | 0.97 | 0.96 | 1.00 | 0.93 | 0.92 | 0.91 | 0.87 | 0.83 |
| Ig_CD69 | 0.88 | 0.92 | 0.93 | 1.00 | 0.92 | 0.90 | 0.80 | 0.79 |
| Ig_CD45R | 0.91 | 0.95 | 0.92 | 0.92 | 1.00 | 0.97 | 0.83 | 0.86 |
| Ig_CD45R | 0.91 | 0.93 | 0.91 | 0.90 | 0.97 | 1.00 | 0.86 | 0.83 |
| Ig_INDO | 0.83 | 0.87 | 0.87 | 0.80 | 0.83 | 0.86 | 1.00 | 0.80 |
| Ig_FOXP3 | 0.85 | 0.82 | 0.83 | 0.79 | 0.86 | 0.83 | 0.80 | 1.00 |
| Ig_CD20 | 0.76 | 0.83 | 0.83 | 0.86 | 0.78 | 0.69 | 0.66 | 0.65 |
| Ig_CXCR3 | 0.84 | 0.88 | 0.89 | 0.83 | 0.82 | 0.82 | 0.78 | 0.73 |
| Ig_CXCL1 [symbol] | 0.79 | 0.77 | 0.81 | 0.69 | 0.68 | 0.75 | 0.82 | 0.64 |
| Ig_CXCL1 [symbol] | 0.72 | 0.68 | 0.70 | 0.67 | 0.64 | 0.70 | 0.74 | 0.59 |
| Ig_CD3E | 0.94 | 0.91 | 0.95 | 0.93 | 0.87 | 0.86 | 0.76 | 0.75 |
| Ig_CD8A | 0.92 | 0.89 | 0.93 | 0.88 | 0.85 | 0.87 | 0.82 | 0.81 |
| Ig_TBX21 | 0.92 | 0.92 | 0.97 | 0.87 | 0.86 | 0.87 | 0.82 | 0.72 |
| Ig_ICOS | 0.89 | 0.90 | 0.93 | 0.87 | 0.83 | 0.84 | 0.78 | 0.68 |
| Ig_IFNG | 0.82 | 0.88 | 0.89 | 0.83 | 0.78 | 0.82 | 0.87 | 0.71 |
| Ig_PRF1 | 0.91 | 0.89 | 0.92 | 0.88 | 0.84 | 0.88 | 0.83 | 0.81 |
| Ig_GZMB | 0.91 | 0.87 | 0.93 | 0.87 | 0.83 | 0.85 | 0.80 | 0.77 |
| Ig_GNLY | 0.86 | 0.83 | 0.87 | 0.87 | 0.80 | 0.82 | 0.78 | 0.78 |
| Ig_FASLG | 0.86 | 0.83 | 0.87 | 0.86 | 0.79 | 0.85 | 0.75 | 0.68 |
| Ig_CCL5 | 0.88 | 0.88 | 0.92 | 0.87 | 0.80 | 0.84 | 0.81 | 0.68 |

| Variable | Ig_CD20 | Ig_CXCR3 | Ig_CXCL10 | Ig_CXCL11 | Ig_CD3E | Ig_CD8A | Ig_TBX21 | Ig_ICOS |
|---|---|---|---|---|---|---|---|---|
| Ig_IL7R | 0.87 | 0.84 | 0.65 | 0.54 | 0.86 | 0.77 | 0.91 | 0.85 |
| Ig_CD3D | 0.82 | 0.89 | 0.84 | 0.74 | 0.94 | 0.93 | 0.97 | 0.94 |
| Ig_CD52 | 0.88 | 0.82 | 0.68 | 0.60 | 0.92 | 0.82 | 0.93 | 0.87 |
| Ig_UBD | 0.69 | 0.85 | 0.92 | 0.81 | 0.81 | 0.89 | 0.90 | 0.89 |
| Ig_GPR17 | 0.67 | 0.74 | 0.76 | 0.75 | 0.85 | 0.88 | 0.83 | 0.78 |
| Ig_GMZK | 0.74 | 0.86 | 0.83 | 0.74 | 0.87 | 0.91 | 0.87 | 0.87 |
| Ig_PRKC [symbol] | 0.74 | 0.79 | 0.72 | 0.71 | 0.88 | 0.91 | 0.81 | 0.78 |
| Ig_STAT4 | 0.74 | 0.78 | 0.73 | 0.77 | 0.83 | 0.87 | 0.81 | 0.79 |
| Ig_TRDV2 | 0.76 | 0.84 | 0.79 | 0.72 | 0.94 | 0.92 | 0.92 | 0.89 |
| Ig_TRAT1 | 0.83 | 0.88 | 0.77 | 0.68 | 0.91 | 0.89 | 0.92 | 0.90 |
| Ig_TRBV1 | 0.83 | 0.89 | 0.81 | 0.70 | 0.95 | 0.93 | 0.97 | 0.93 |
| Ig_CD69 | 0.86 | 0.83 | 0.69 | 0.67 | 0.93 | 0.88 | 0.87 | 0.87 |
| Ig_CD45R | 0.78 | 0.82 | 0.68 | 0.64 | 0.87 | 0.85 | 0.86 | 0.83 |
| Ig_CD45R | 0.69 | 0.82 | 0.75 | 0.70 | 0.86 | 0.87 | 0.87 | 0.84 |
| Ig_INDO | 0.66 | 0.78 | 0.82 | 0.74 | 0.76 | 0.82 | 0.82 | 0.78 |
| Ig_FOXP3 | 0.65 | 0.73 | 0.64 | 0.59 | 0.75 | 0.81 | 0.72 | 0.68 |
| Ig_CD20 | 1.00 | 0.73 | 0.51 | 0.47 | 0.82 | 0.68 | 0.79 | 0.74 |
| Ig_CXCR3 | 0.73 | 1.00 | 0.81 | 0.73 | 0.87 | 0.89 | 0.89 | 0.90 |
| Ig_CXCL1 [symbol] | 0.51 | 0.81 | 1.00 | 0.90 | 0.73 | 0.87 | 0.81 | 0.84 |
| Ig_CXCL1 [symbol] | 0.47 | 0.73 | 0.90 | 1.00 | 0.65 | 0.81 | 0.67 | 0.72 |
| Ig_CD3E | 0.82 | 0.87 | 0.73 | 0.65 | 1.00 | 0.91 | 0.92 | 0.91 |
| Ig_CD8A | 0.68 | 0.89 | 0.87 | 0.81 | 0.91 | 1.00 | 0.88 | 0.90 |
| Ig_TBX21 | 0.79 | 0.89 | 0.81 | 0.67 | 0.92 | 0.88 | 1.00 | 0.95 |
| Ig_ICOS | 0.74 | 0.90 | 0.84 | 0.72 | 0.91 | 0.90 | 0.95 | 1.00 |
| Ig_IFNG | 0.67 | 0.82 | 0.89 | 0.73 | 0.83 | 0.89 | 0.87 | 0.90 |
| Ig_PRF1 | 0.68 | 0.85 | 0.86 | 0.79 | 0.91 | 0.98 | 0.87 | 0.88 |
| Ig_GZMB | 0.71 | 0.85 | 0.80 | 0.75 | 0.90 | 0.92 | 0.89 | 0.90 |
| Ig_GNLY | 0.68 | 0.77 | 0.74 | 0.69 | 0.89 | 0.89 | 0.80 | 0.83 |
| Ig_FASLG | 0.63 | 0.78 | 0.83 | 0.81 | 0.87 | 0.93 | 0.85 | 0.88 |
| Ig_CCL5 | 0.68 | 0.89 | 0.91 | 0.81 | 0.91 | 0.96 | 0.91 | 0.94 |

| Variable | Ig_IFNG | Ig_PRF1 | Ig_GZMB | Ig_GNLY | Ig_FASLG | Ig_CCL5 |
|---|---|---|---|---|---|---|
| Ig_IL7R | 0.75 | 0.76 | 0.80 | 0.74 | 0.68 | 0.77 |
| Ig_CD3D | 0.91 | 0.92 | 0.92 | 0.87 | 0.87 | 0.93 |
| Ig_CD52 | 0.78 | 0.82 | 0.89 | 0.84 | 0.78 | 0.83 |
| Ig_UBD | 0.92 | 0.89 | 0.86 | 0.77 | 0.85 | 0.91 |
| Ig_GPR17 | 0.74 | 0.88 | 0.90 | 0.85 | 0.82 | 0.83 |
| Ig_GMZK | 0.92 | 0.91 | 0.84 | 0.83 | 0.85 | 0.92 |
| Ig_PRKC [symbol] | 0.75 | 0.91 | 0.88 | 0.88 | 0.81 | 0.81 |
| Ig_STAT4 | 0.78 | 0.87 | 0.87 | 0.82 | 0.81 | 0.82 |
| Ig_TRDV2 | 0.82 | 0.91 | 0.91 | 0.86 | 0.86 | 0.88 |
| Ig_TRAT1 | 0.88 | 0.89 | 0.87 | 0.83 | 0.83 | 0.88 |
| Ig_TRBV1 | 0.89 | 0.92 | 0.93 | 0.87 | 0.87 | 0.92 |
| Ig_CD69 | 0.83 | 0.88 | 0.87 | 0.87 | 0.86 | 0.87 |
| Ig_CD45R | 0.78 | 0.84 | 0.83 | 0.80 | 0.79 | 0.80 |
| Ig_CD45R | 0.82 | 0.88 | 0.85 | 0.82 | 0.85 | 0.84 |
| Ig_INDO | 0.87 | 0.83 | 0.80 | 0.78 | 0.75 | 0.81 |
| Ig_FOXP3 | 0.71 | 0.81 | 0.77 | 0.78 | 0.68 | 0.68 |
| Ig_CD20 | 0.67 | 0.68 | 0.71 | 0.68 | 0.63 | 0.68 |
| Ig_CXCR3 | 0.82 | 0.85 | 0.82 | 0.77 | 0.78 | 0.89 |
| Ig_CXCL1 [symbol] | 0.89 | 0.86 | 0.80 | 0.74 | 0.83 | 0.91 |
| Ig_CXCL1 [symbol] | 0.73 | 0.79 | 0.75 | 0.69 | 0.81 | 0.81 |
| Ig_CD3E | 0.83 | 0.91 | 0.90 | 0.89 | 0.87 | 0.91 |
| Ig_CD8A | 0.89 | 0.98 | 0.92 | 0.89 | 0.93 | 0.96 |
| Ig_TBX21 | 0.87 | 0.87 | 0.89 | 0.80 | 0.85 | 0.91 |
| Ig_ICOS | 0.90 | 0.88 | 0.90 | 0.83 | 0.88 | 0.94 |
| Ig_IFNG | 1.00 | 0.90 | 0.85 | 0.82 | 0.84 | 0.93 |
| Ig_PRF1 | 0.90 | 1.00 | 0.94 | 0.92 | 0.93 | 0.95 |
| Ig_GZMB | 0.85 | 0.94 | 1.00 | 0.92 | 0.91 | 0.92 |
| Ig_GNLY | 0.82 | 0.92 | 0.92 | 1.00 | 0.86 | 0.88 |
| Ig_FASLG | 0.84 | 0.93 | 0.91 | 0.86 | 1.00 | 0.93 |
| Ig_CCL5 | 0.93 | 0.95 | 0.92 | 0.88 | 0.93 | 1.00 |

[symbol]

indicates data missing or illegible when filed

TABLE 9A
18 best models obtained by logistic regression.

| Predictor | R2 (%) | Chi-square Score |
|---|---|---|
| PRF1 | 55 | 16 |
| IRF1 | 50 | 14 |
| GZMB | 46 | 14 |
| GNLY | 45 | 14 |
| CD8A | 46 | 14 |
| PRKCQ | 45 | 14 |
| FOXP3 | 42 | 13 |
| IFNG | 52 | 13 |
| CCL5 | 45 | 13 |
| GPR171 | 41 | 12 |
| TRBV19 | 44 | 11 |
| CD3E | 39 | 11 |
| TBX21 | 44 | 11 |
| FASLG | 39 | 11 |
| CXCL10 | 35 | 10 |
| ICOS | 39 | 10 |
| CXCR3 | 35 | 9 |
| CXCL11 | 22 | 6 |

TABLE 10
Percentage of correct classification calculated using the
logistic regression model for some genes.

| Model | Percentage of patients correctly classified |
|---|---|
| PRF1 | 86.6% |
| IRF1 | 89.7% |
| GZMB | 82.8% |
| GNLY | 82.8% |
| CD8A | 82.8% |
| PRKCQ | 89.7% |
| FOXP3 | 86.2% |
| IFNG | 89.7% |
| CCL5 | 79.3% |
| GPR171 | 82.8% |
| TRBV19 | 86.2% |
| CDE3 | 75.9% |
| TBX21 | 82.8% |
| FASLG | 75.9% |
| CXCL10 | 75.9% |
| ICOS | 75.9% |
| CXR3 | 75.9% |
| CXCL11 | 72.4% |

TABLE 11A

| Probe | | Gene | Patient Identity Number | | | | | | | | | |
|---|---|---|---|---|---|---|---|---|---|---|---|---|
| Set | Gene Title | Symbol | 14 | 52 | 66 | 37 | 16 | 13 | 55 | 56 | 5 | 60 |
| 1555759_a_at | chemokine (C-C | CCL5 | 3.148 | 3.204 | 3.208 | 3.096 | 3.055 | 3.049 | 3.096 | 3.208 | 2.664 | 2.835 |
| | motif) ligand 5 | | | | | | | | | | | |
| 1555834_at | Ubiquitin | UCHL1 | 7.911 | 2.4 | 2.324 | 5.169 | 5.06 | 2.401 | 3.561 | 2.627 | 4.868 | 2.401 |
| | carboxyl- | | | | | | | | | | | |
| | terminal esterase | | | | | | | | | | | |
| | L1 (ubiquitin | | | | | | | | | | | |
| | thiolesterase) | | | | | | | | | | | |
| 1558290_a_at | Pvt1 oncogene | PVT1 | 10.849 | 10.579 | 11.312 | 10.154 | 9.815 | 10.752 | 11.192 | 9.514 | 10.132 | 8.752 |
| | homolog, MYC | /// | | | | | | | | | | |
| | activator | LOC441378 | | | | | | | | | | |
| | (mouse) /// | | | | | | | | | | | |
| | LOC441378 | | | | | | | | | | | |
| 1569942_at | CDNA clone | — | 5.295 | 3.843 | 4.837 | 5.187 | 3.748 | 5.656 | 3.518 | 6.042 | 6.463 | 4.378 |
| | IMAGE: 4796388 | | | | | | | | | | | |
| 204661_at | CD52 antigen | CD52 | 5.673 | 5.506 | 8.56 | 5.104 | 5.814 | 5.755 | 4.087 | 4.785 | 3.839 | 4.29 |
| | (CAMPATH-1 | | | | | | | | | | | |
| | antigen) /// | | | | | | | | | | | |
| | CD52 antigen | | | | | | | | | | | |
| | (CAMPATH-1 | | | | | | | | | | | |
| | antigen) | | | | | | | | | | | |
| 205890_s_at | ubiquitin D | UBD | 4.731 | 7.003 | 9.226 | 4.138 | 3.687 | 8.103 | 3.242 | 6.126 | 3.231 | 7.706 |
| 206118_at | signal | STAT4 | 4.336 | 3.291 | 4.142 | 2.396 | 3.235 | 3.291 | 2.382 | 2.439 | 2.663 | 2.948 |
| | transducer and | | | | | | | | | | | |
| | activator of | | | | | | | | | | | |
| | transcription 4 | | | | | | | | | | | |
| 206666_at | granzyme K | GZMK | 6.187 | 5.357 | 7.079 | 2.659 | 2.571 | 5.033 | 2.225 | 3.383 | 2.567 | 3.104 |
| | (granzyme 3; | | | | | | | | | | | |
| | tryptase II) /// | | | | | | | | | | | |
| | granzyme K | | | | | | | | | | | |
| | (granzyme 3; | | | | | | | | | | | |
| | tryptase II) | | | | | | | | | | | |
| 207072_at | interleukin 18 | IL18RAP | 2.225 | 2.237 | 2.232 | 2.232 | 2.231 | 2.232 | 2.227 | 2.237 | 2.227 | 2.237 |
| | receptor | | | | | | | | | | | |
| | accessory | | | | | | | | | | | |
| | protein | | | | | | | | | | | |
| 207651_at | G protein- | GPR171 | 3.961 | 7.086 | 4.363 | 4.635 | 3.271 | 5.499 | 3.426 | 4.679 | 3.42 | 4.672 |
| | coupled receptor | | | | | | | | | | | |
| | 171 | | | | | | | | | | | |
| 209606_at | pleckstrin | PSCDBP | 4.553 | 5.031 | 5.888 | 4.679 | 3.194 | 3.996 | 3.036 | 3.285 | 3.692 | 3.473 |
| | homology, Sec7 | | | | | | | | | | | |
| | and coiled-coil | | | | | | | | | | | |
| | domains, | | | | | | | | | | | |
| | binding protein | | | | | | | | | | | |
| | /// pleckstrin | | | | | | | | | | | |
| | homology, Sec7 | | | | | | | | | | | |
| | and coiled-coil | | | | | | | | | | | |
| | domains, | | | | | | | | | | | |
| | binding protein | | | | | | | | | | | |
| 209949_at | neutrophil | NCF2 | 6.058 | 6.184 | 3.286 | 6.305 | 4.585 | 3.866 | 4.672 | 3.294 | 4.457 | 3.802 |
| | cytosolic factor | | | | | | | | | | | |
| | 2 (65 kDa, | | | | | | | | | | | |
| | chronic | | | | | | | | | | | |
| | granulomatous | | | | | | | | | | | |
| | disease, | | | | | | | | | | | |
| | autosomal 2) | | | | | | | | | | | |
| 210038_at | protein kinase | PRKCQ | 2.362 | 2.18 | 2.415 | 2.178 | 2.364 | 2.358 | 2.577 | 2.181 | 2.179 | 2.191 |
| | C, theta | | | | | | | | | | | |
| 210629_x_at | leukocyte | LST1 | 5.649 | 6.559 | 6.72 | 5.368 | 5.367 | 3.893 | 3.484 | 6.134 | 4.775 | 5.283 |
| | specific | | | | | | | | | | | |
| | transcript 1 | | | | | | | | | | | |
| 210972_x_at | T cell receptor | TRA@ | 4.292 | 6.159 | 7.116 | 3.791 | 4.059 | 6.091 | 5.234 | 5.057 | 4.291 | 4.842 |
| | alpha locus /// T | /// | | | | | | | | | | |
| | cell receptor | TRDV2 | | | | | | | | | | |
| | delta variable 2 | /// | | | | | | | | | | |
| | /// T cell | TRAV20 | | | | | | | | | | |
| | receptor alpha | /// | | | | | | | | | | |
| | variable 20 /// T | TRAJ17 | | | | | | | | | | |
| | cell receptor | /// | | | | | | | | | | |
| | alpha joining 17 | TRAC | | | | | | | | | | |
| | /// T cell | | | | | | | | | | | |
| | receptor alpha | | | | | | | | | | | |
| | constant | | | | | | | | | | | |
| 211144_x_at | T cell receptor | TRGC2 | 2.887 | 2.261 | 4.452 | 2.257 | 2.193 | 3.203 | 2.347 | 2.453 | 2.332 | 2.196 |
| | gamma constant 2 | | | | | | | | | | | |
| 211796_s_at | T cell receptor | TRBV21- | 3.275 | 3.556 | 7.359 | 2.877 | 2.424 | 5.177 | 2.521 | 2.728 | 2.297 | 2.909 |
| | beta variable 21- | 1 /// | | | | | | | | | | |
| | 1 /// T cell | TRBV19 | | | | | | | | | | |
| | receptor beta | /// | | | | | | | | | | |
| | variable 19 /// T | TRBV5- | | | | | | | | | | |
| | cell receptor | 4 /// | | | | | | | | | | |
| | beta variable 5-4 | TRBV3- | | | | | | | | | | |
| | /// T cell | 1 /// | | | | | | | | | | |
| | receptor beta | TRBC1 | | | | | | | | | | |
| | variable 3-1 /// T | | | | | | | | | | | |
| | cell receptor | | | | | | | | | | | |
| | beta constant 1 | | | | | | | | | | | |
| 212671_s_at | major | HLA- | 8.1 | 7.479 | 6.553 | 8.223 | 6.574 | 7.356 | 7.347 | 5.884 | 6.068 | 5.558 |
| | histocompatibility | DQA1 | | | | | | | | | | |
| | complex, class | /// | | | | | | | | | | |
| | II, DQ alpha 1 | HLA- | | | | | | | | | | |
| | /// major | DQA2 | | | | | | | | | | |
| | histocompatibility | | | | | | | | | | | |
| | complex, class | | | | | | | | | | | |
| | II, DQ alpha 2 | | | | | | | | | | | |
| 213193_x_at | T cell receptor | TRBV19 | 4.185 | 3.787 | 7.027 | 3.011 | 2.962 | 4.53 | 3.096 | 3.192 | 2.829 | 3.058 |
| | beta variable 19 | /// | | | | | | | | | | |
| | /// T cell | TRBC1 | | | | | | | | | | |
| | receptor beta | | | | | | | | | | | |
| | variable 19 /// T | | | | | | | | | | | |
| | cell receptor | | | | | | | | | | | |
| | beta constant 1 | | | | | | | | | | | |
| | /// T cell | | | | | | | | | | | |
| | receptor beta | | | | | | | | | | | |
| | constant 1 | | | | | | | | | | | |
| 213539_at | CD3D antigen, | CD3D | 3.179 | 4.399 | 7.52 | 3.352 | 3.182 | 5.87 | 2.8 | 3.183 | 3.178 | 3.626 |
| | delta | | | | | | | | | | | |
| | polypeptide | | | | | | | | | | | |
| | (TiT3 complex) | | | | | | | | | | | |
| 213560_at | Growth arrest | GADD45B | 4.967 | 5.948 | 6.218 | 5.528 | 4.673 | 5.367 | 5.49 | 3.67 | 5.735 | 5.048 |
| | and DNA- | | | | | | | | | | | |
| | damage- | | | | | | | | | | | |
| | inducible, beta | | | | | | | | | | | |
| 216261_at | integrin, beta 3 | ITGB3 | 8.21 | 2.588 | 4.394 | 3.536 | 6.237 | 4.486 | 3.088 | 6.729 | 6.77 | 2.606 |
| | (platelet | | | | | | | | | | | |
| | glycoprotein | | | | | | | | | | | |
| | IIIa, antigen | | | | | | | | | | | |
| | CD61) | | | | | | | | | | | |
| 217147_s_at | T cell receptor | TRAT1 | 3.914 | 4.251 | 5.453 | 2.871 | 2.292 | 4.283 | 2.327 | 2.887 | 2.277 | 2.308 |
| | associated | | | | | | | | | | | |
| | transmembrane | | | | | | | | | | | |
| | adaptor 1 | | | | | | | | | | | |
| 217478_s_at | major | HLA- | 8.989 | 8.655 | 8.599 | 9.516 | 8.621 | 7.051 | 8.181 | 8.129 | 7.41 | 8.04 |
| | histocompatibility | DMA | | | | | | | | | | |
| | complex, class | | | | | | | | | | | |
| | II, DM alpha | | | | | | | | | | | |
| 218950_at | centaurin, delta 3 | CENTD3 | 9.212 | 7.139 | 8.794 | 8.51 | 8.32 | 7.438 | 9.077 | 8.075 | 9.012 | 7.142 |
| 220330_s_at | SAM domain, | SAMSN1 | 4.712 | 4.228 | 5.117 | 4.641 | 4.804 | 3.557 | 2.702 | 4.246 | 3.492 | 3.828 |
| | SH3 domain and | | | | | | | | | | | |
| | nuclear | | | | | | | | | | | |
| | localisation | | | | | | | | | | | |
| | signals, 1 | | | | | | | | | | | |
| 222934_s_at | C-type lectin | CLEC4E | 5.382 | 4.219 | 2.931 | 2.753 | 2.511 | 3.812 | 4.494 | 2.512 | 3.917 | 3.417 |
| | domain family | | | | | | | | | | | |
| | 4, member E | | | | | | | | | | | |
| 223827_at | tumor necrosis | TNFRSF19 | 4.16 | 3.985 | 5.98 | 2.86 | 5.91 | 3.239 | 4.301 | 6.268 | 5.029 | 4.931 |
| | factor receptor | | | | | | | | | | | |
| | superfamily, | | | | | | | | | | | |
| | member 19 | | | | | | | | | | | |
| 224771_at | neuron | NAV1 | 7.893 | 6.773 | 7.352 | 7.998 | 8.385 | 7.571 | 7.077 | 7.277 | 6.956 | 7.304 |
| | navigator 1 | | | | | | | | | | | |
| 224772_at | neuron | NAV1 | 10.827 | 9.319 | 9.458 | 9.799 | 10.771 | 10.304 | 9.47 | 9.943 | 9.752 | 9.998 |
| | navigator 1 | | | | | | | | | | | |
| 224773_at | neuron | NAV1 | 8.959 | 7.241 | 7.742 | 9.332 | 9.582 | 8.051 | 8.427 | 8.479 | 7.795 | 7.564 |
| | navigator 1 | | | | | | | | | | | |
| 224774_s_at | neuron | NAV1 | 7.288 | 6.204 | 7.292 | 8.109 | 7.683 | 6.486 | 7.056 | 5.947 | 6.406 | 6.181 |
| | navigator 1 | | | | | | | | | | | |
| 227145_at | lysyl oxidase- | LOXL4 | 11.368 | 8.235 | 10.93 | 12.565 | 12.636 | 12.563 | 7.768 | 11.858 | 12.092 | 11.62 [symbol] |
| | like 4 | | | | | | | | | | | |
| 227584_at | Neuron | NAV1 | 11.735 | 10.222 | 10.601 | 10.533 | 11.778 | 11.041 | 10.121 | 11.247 | 10.732 | 11.00 [symbol] |
| | navigator 1 | | | | | | | | | | | |
| 227812_at | tumor necrosis | TNFRSF19 | 11.9 | 11.787 | 12.618 | 10.566 | 12.747 | 11.729 | 11.115 | 12.911 | 12.125 | 11.83 [symbol] |
| | factor receptor | | | | | | | | | | | |
| | superfamily, | | | | | | | | | | | |
| | member 19 | | | | | | | | | | | |
| 229723_at | T-cell activation | TAGAP | 5.224 | 4.874 | 5.527 | 5.091 | 2.501 | 3.01 | 4.398 | 2.891 | 3.472 | 3.425 |
| | GTPase | | | | | | | | | | | |
| | activating | | | | | | | | | | | |
| | protein | | | | | | | | | | | |
| 232024_at | GTPase, IMAP | GIMAP2 | 8.489 | 7.321 | 7.275 | 6.411 | 7.83 | 6.861 | 6.317 | 8.172 | 7.42 | 7.119 |
| | family member 2 | | | | | | | | | | | |
| 238544_at | Insulin-like | IGF1R | 6.79 | 8.031 | 9.43 | 5.969 | 7.331 | 7.827 | 8.887 | 6.322 | 9.011 | 7.444 |
| | growth factor 1 | | | | | | | | | | | |
| | receptor | | | | | | | | | | | |
| 242886_at | Chromosome 17 | C17orf63 | 10.066 | 10.766 | 9.654 | 8.976 | 9.813 | 9.864 | 10.039 | 9.839 | 8.819 | 9.808 |
| | open reading | | | | | | | | | | | |
| | frame 63 | | | | | | | | | | | |
| 242986_at | Neuron | NAV1 | 9.936 | 9.155 | 9.341 | 10.292 | 10.071 | 9.41 | 9.098 | 9.552 | 8.465 | 8.663 |
| | navigator 1 | | | | | | | | | | | |
| 34210_at | CD52 antigen | CD52 | 6.58 | 6.581 | 9.446 | 5.799 | 6.518 | 6.798 | 4.246 | 6.008 | 3.469 | 5.289 |
| | (CAMPATH-1 | | | | | | | | | | | |
| | antigen) | | | | | | | | | | | |

[symbol]

indicates data missing or illegible when filed

TABLE 11B

| Probe | | Gene | Patient Identity Number | | | | |
|---|---|---|---|---|---|---|---|
| Set | Gene Title | Symbol | 75 | 2 | 65 | 20 | 10 |
| 1555759_a_at | chemokine | CCL5 | 4.665 | 5.498 | 7.109 | 3.207 | 5.121 |
| | (C-C motif) | | | | | | |
| | ligand 5 | | | | | | |
| 1555834_at | Ubiquitin | UCHL1 | 2.329 | 2.36 | 2.4 | 2.399 | 2.358 |
| | carboxyl- | | | | | | |
| | terminal | | | | | | |
| | esterase L1 | | | | | | |
| | (ubiquitin | | | | | | |
| | thiolesterase) | | | | | | |
| 1558290_a_at | Pvt1 | PVT1 | 8.59 | 7.403 | 8.63 | 8.588 | 9.536 |
| | oncogene | /// | | | | | |
| | homolog, | LOC441378 | | | | | |
| | MYC | | | | | | |
| | activator | | | | | | |
| | (mouse) /// | | | | | | |
| | LOC441378 | | | | | | |
| 1569942_at | CDNA clone | — | 3.605 | 3.324 | 4.066 | 2.849 | 4.189 |
| | IMAGE: 4796388 | | | | | | |
| 204661_at | CD52 antigen | CD52 | 8.008 | 8.301 | 9.702 | 8.979 | 9.415 |
| | (CAMPATH- | | | | | | |
| | 1 antigen) /// | | | | | | |
| | CD52 antigen | | | | | | |
| | (CAMPATH- | | | | | | |
| | 1 antigen) | | | | | | |
| 205890_s_at | ubiquitin D | UBD | 11.687 | 10.159 | 11.582 | 7.52 | 9.692 |
| 206118_at | signal | STAT4 | 5.794 | 5.481 | 4.044 | 5.157 | 6.026 |
| | transducer | | | | | | |
| | and activator | | | | | | |
| | of | | | | | | |
| | transcription 4 | | | | | | |
| 206666_at | granzyme K | GZMK | 7.615 | 9.057 | 9.338 | 6.513 | 8.013 |
| | (granzyme 3; | | | | | | |
| | tryptase II) /// | | | | | | |
| | granzyme K | | | | | | |
| | (granzyme 3; | | | | | | |
| | tryptase II) | | | | | | |
| 207072_at | interleukin 18 | IL18RAP | 4.76 | 2.358 | 2.233 | 3.154 | 2.235 |
| | receptor | | | | | | |
| | accessory | | | | | | |
| | protein | | | | | | |
| 207651_at | G protein- | GPR171 | 8.491 | 8.969 | 8.37 | 6.884 | 8.501 |
| | coupled | | | | | | |
| | receptor 171 | | | | | | |
| 209606_at | pleckstrin | PSCDBP | 6.808 | 6.788 | 8.373 | 6.524 | 7.997 |
| | homology, | | | | | | |
| | Sec7 and | | | | | | |
| | coiled-coil | | | | | | |
| | domains, | | | | | | |
| | binding | | | | | | |
| | protein /// | | | | | | |
| | pleckstrin | | | | | | |
| | homology, | | | | | | |
| | Sec7 and | | | | | | |
| | coiled-coil | | | | | | |
| | domains, | | | | | | |
| | binding | | | | | | |
| | protein | | | | | | |
| 209949_at | neutrophil | NCF2 | 6.928 | 6.845 | 7.973 | 5.572 | 5.428 |
| | cytosolic | | | | | | |
| | factor 2 | | | | | | |
| | (65 kDa, | | | | | | |
| | chronic | | | | | | |
| | granulomatous | | | | | | |
| | disease, | | | | | | |
| | autosomal 2) | | | | | | |
| 210038_at | protein | PRKCQ | 4.981 | 4.768 | 2.406 | 2.836 | 6.907 |
| | kinase C, | | | | | | |
| | theta | | | | | | |
| 210629_x_at | leukocyte | LST1 | 7.078 | 6.711 | 6.926 | 5.831 | 6.424 |
| | specific | | | | | | |
| | transcript 1 | | | | | | |
| 210972_x_at | T cell | TRA@ | 7.913 | 8.072 | 11.25 | 6.451 | 10.646 |
| | receptor | /// | | | | | |
| | alpha locus | TRDV2 | | | | | |
| | /// T cell | /// | | | | | |
| | receptor delta | TRAV20 | | | | | |
| | variable 2 /// | /// | | | | | |
| | T cell | TRAJ17 | | | | | |
| | receptor | /// | | | | | |
| | alpha | TRAC | | | | | |
| | variable 20 /// | | | | | | |
| | T cell | | | | | | |
| | receptor | | | | | | |
| | alpha joining | | | | | | |
| | 17 /// T cell | | | | | | |
| | receptor | | | | | | |
| | alpha | | | | | | |
| | constant | | | | | | |
| 211144_x_at | T cell | TRGC2 | 6.096 | 4.892 | 6.456 | 2.624 | 6.163 |
| | receptor | | | | | | |
| | gamma | | | | | | |
| | constant 2 | | | | | | |
| 211796_s_at | T cell | TRBV21- | 7.924 | 8.11 | 10.708 | 6.84 | 9.737 |
| | receptor beta | 1 /// | | | | | |
| | variable 21-1 | TRBV19 | | | | | |
| | /// T cell | /// | | | | | |
| | receptor beta | TRBV5- | | | | | |
| | variable 19 /// | 4 /// | | | | | |
| | T cell | TRBV3- | | | | | |
| | receptor beta | 1 /// | | | | | |
| | variable 5-4 | TRBC1 | | | | | |
| | /// T cell | | | | | | |
| | receptor beta | | | | | | |
| | variable 3-1 | | | | | | |
| | /// T cell | | | | | | |
| | receptor beta | | | | | | |
| | constant 1 | | | | | | |
| 212671_s_at | major | HLA- | 10.798 | 9.601 | 11.257 | 9.336 | 8.87 |
| | histocompatibility | DQA1 | | | | | |
| | complex, | /// | | | | | |
| | class II, DQ | HLA- | | | | | |
| | alpha 1 /// | DQA2 | | | | | |
| | major | | | | | | |
| | histocompatibility | | | | | | |
| | complex, | | | | | | |
| | class II, DQ | | | | | | |
| | alpha 2 | | | | | | |
| 213193_x_at | T cell | TRBV19 | 7.553 | 7.871 | 10.191 | 6.704 | 9.248 |
| | receptor beta | /// | | | | | |
| | variable 19 /// | TRBC1 | | | | | |
| | T cell | | | | | | |
| | receptor beta | | | | | | |
| | variable 19 /// | | | | | | |
| | T cell | | | | | | |
| | receptor beta | | | | | | |
| | constant 1 /// | | | | | | |
| | T cell | | | | | | |
| | receptor beta | | | | | | |
| | constant 1 | | | | | | |
| 213539_at | CD3D | CD3D | 8.208 | 8.038 | 9.64 | 7.257 | 8.387 |
| | antigen, delta | | | | | | |
| | polypeptide | | | | | | |
| | (TiT3 | | | | | | |
| | complex) | | | | | | |
| 213560_at | Growth arrest | GADD45B | 7.243 | 5.936 | 6.794 | 7.57 | 6.389 |
| | and DNA- | | | | | | |
| | damage- | | | | | | |
| | inducible, | | | | | | |
| | beta | | | | | | |
| 216261_at | integrin, beta | ITGB3 | 2.545 | 2.691 | 2.651 | 2.592 | 2.59 |
| | 3 (platelet | | | | | | |
| | glycoprotein | | | | | | |
| | IIIa, antigen | | | | | | |
| | CD61) | | | | | | |
| 217147_s_at | T cell | TRAT1 | 6.748 | 6.139 | 8.028 | 5.044 | 7.377 |
| | receptor | | | | | | |
| | associated | | | | | | |
| | transmembrane | | | | | | |
| | adaptor 1 | | | | | | |
| 217478_s_at | major | HLA- | 10.873 | 10.838 | 11.541 | 9.389 | 10.193 |
| | histocompatibility | DMA | | | | | |
| | complex, | | | | | | |
| | class II, DM | | | | | | |
| | alpha | | | | | | |
| 218950_at | centaurin, | CENTD3 | 7.911 | 7.983 | 5.552 | 6.866 | 7.456 |
| | delta 3 | | | | | | |
| 220330_s_at | SAM | SAMSN1 | 7.494 | 6.244 | 4.271 | 5.243 | 7.151 |
| | domain, SH3 | | | | | | |
| | domain and | | | | | | |
| | nuclear | | | | | | |
| | localisation | | | | | | |
| | signals, 1 | | | | | | |
| 222934_s_at | C-type lectin | CLEC4E | 6.912 | 6.523 | 6.984 | 5.384 | 4.588 |
| | domain | | | | | | |
| | family 4, | | | | | | |
| | member E | | | | | | |
| 223827_at | tumor | TNFRSF19 | 3.043 | 3.308 | 2.721 | 2.855 | 3.48 |
| | necrosis | | | | | | |
| | factor | | | | | | |
| | receptor | | | | | | |
| | superfamily, | | | | | | |
| | member 19 | | | | | | |
| 224771_at | neuron | NAV1 | 6.378 | 7.607 | 6.624 | 6.24 | 6.483 |
| | navigator 1 | | | | | | |
| 224772_at | neuron | NAV1 | 9.184 | 10.073 | 8.059 | 7.897 | 8.937 |
| | navigator 1 | | | | | | |
| 224773_at | neuron | NAV1 | 7.427 | 7.781 | 5.878 | 5.855 | 6.881 |
| | navigator 1 | | | | | | |
| 224774_s_at | neuron | NAV1 | 5.706 | 7.087 | 5.432 | 5.724 | 5.939 |
| | navigator 1 | | | | | | |
| 227145_at | lysyl oxidase- | LOXL4 | 3.699 | 7.798 | 3.703 | 4.953 | 8.792 |
| | like 4 | | | | | | |
| 227584_at | Neuron | NAV1 | 10.236 | 10.828 | 8.45 | 9.758 | 9.512 |
| | navigator 1 | | | | | | |
| 227812_at | tumor | TNFRSF19 | 9.962 | 9.478 | 9.706 | 8.939 | 11.568 |
| | necrosis | | | | | | |
| | factor | | | | | | |
| | receptor | | | | | | |
| | superfamily, | | | | | | |
| | member 19 | | | | | | |
| 229723_at | T-cell | TAGAP | 6.398 | 6.009 | 9.043 | 7.032 | 8.532 |
| | activation | | | | | | |
| | GTPase | | | | | | |
| | activating | | | | | | |
| | protein | | | | | | |
| 232024_at | GTPasc, | GIMAP2 | 9.137 | 9.127 | 8.794 | 7.948 | 9.093 |
| | IMAP family | | | | | | |
| | member 2 | | | | | | |
| 238544_at | Insulin-like | IGF1R | 7.023 | 6.644 | 6.255 | 4.359 | 6.899 |
| | growth factor | | | | | | |
| | 1 receptor | | | | | | |
| 242886_at | Chromosome | C17orf63 | 9.338 | 10.004 | 7.65 | 7.978 | 8.744 |
| | 17 open | | | | | | |
| | reading frame | | | | | | |
| | 63 | | | | | | |
| 242986_at | Neuron | NAV1 | 8.777 | 8.689 | 8.154 | 6.863 | 8.461 |
| | navigator 1 | | | | | | |
| 34210_at | CD52 antigen | CD52 | 9.102 | 9.324 | 10.652 | 10.24 | 10.103 |
| | (CAMPATH- | | | | | | |
| | 1 antigen) | | | | | | |

| Probe | Patient Identity Number | | | | | | |
|---|---|---|---|---|---|---|---|
| Set | 19 | 23 | 26 | 27 | 38 | 8 | 67 |
| 1555759_a_at | 3.656 | 4.159 | 4.664 | 5.256 | 5.411 | 3.034 | 3.11 |
| 1555834_at | 2.398 | 2.463 | 2.401 | 2.4 | 2.4 | 5.041 | 2.40 |
| 1558290_a_at | 8.46 | 9.384 | 9.409 | 8.504 | 8.57 | 10.616 | 9.90 [symbol] |
| 1569942_at | 2.198 | 3.972 | 3.61 | 3.392 | 2.944 | 5.832 | 4.38 [symbol] |
| 204661_at | 6.144 | 8.556 | 8.487 | 11.34 | 10.289 | 4.082 | 8.69 [symbol] |
| 205890_s_at | 8.088 | 9.801 | 12.384 | 10.657 | 10.131 | 3.178 | 9.54 |
| 206118_at | 5.775 | 6.731 | 5.845 | 8.349 | 6.709 | 2.383 | 3.09 |
| 206666_at | 7.564 | 8.532 | 8.921 | 8.337 | 9.328 | 2.305 | 6.371 |
| 207072_at | 2.264 | 2.512 | 2.239 | 3.2 | 4.126 | 2.222 | 2.23 [symbol] |
| 207651_at | 8.037 | 8.578 | 10.668 | 10.834 | 9.404 | 3.046 | 6.32 [symbol] |
| 209606_at | 7.366 | 7.301 | 7.101 | 10.455 | 7.959 | 2.232 | 7.231 |
| 209949_at | 6.643 | 7.499 | 6.281 | 6.48 | 7.213 | 3.405 | 7.449 |
| 210038_at | 4.108 | 5.51 | 5.272 | 8.218 | 4.73 | 2.179 | 2.301 |
| 210629_x_at | 6.69 | 7.323 | 6.834 | 7.058 | 8.828 | 3.779 | 7.287 |
| 210972_x_at | 7.331 | 8.326 | 9.727 | 11.544 | 8.3 | 4.292 | 6.411 |
| 211144_x_at | 2.689 | 6.279 | 7.941 | 7.365 | 6.31 | 2.189 | 2.64 [symbol] |
| 211796_s_at | 6.456 | 8.211 | 9.022 | 11.111 | 8.316 | 2.293 | 5.74 [symbol] |
| 212671_s_at | 9.39 | 9.435 | 10.008 | 10.974 | 10.757 | 2.679 | 7.61 |
| 213193_x_at | 5.828 | 7.936 | 8.266 | 11.152 | 7.722 | 2.83 | 5.47 [symbol] |
| 213539_at | 6.635 | 8.381 | 9.075 | 10.519 | 8.841 | 2.311 | 6.07 [symbol] |
| 213560_at | 7.236 | 7.105 | 6.776 | 7.114 | 6.774 | 3.402 | 7.12 [symbol] |
| 216261_at | 2.553 | 2.609 | 4.561 | 2.622 | 2.557 | 6.05 | 2.60 [symbol] |
| 217147_s_at | 7.56 | 6.512 | 7.612 | 9.246 | 8.077 | 2.281 | 3.00 [symbol] |
| 217478_s_at | 9.475 | 10.475 | 9.998 | 11.266 | 10.161 | 7.109 | 9.33 |
| 218950_at | 5.891 | 7.35 | 5.522 | 5.804 | 6.245 | 8.04 | 8.20 [symbol] |
| 220330_s_at | 6.458 | 7.429 | 8.583 | 8.421 | 7.405 | 2.325 | 6.71 [symbol] |
| 222934_s_at | 6.678 | 6.941 | 4.589 | 4.731 | 5.877 | 3.019 | 7.66 [symbol] |
| 223827_at | 2.8 | 2.854 | 2.977 | 2.796 | 3.43 | 4.669 | 2.99 [symbol] |
| 224771_at | 4.678 | 6.467 | 6.537 | 3.882 | 5.375 | 6.798 | 5.73 |
| 224772_at | 8.214 | 8.963 | 9.472 | 7.529 | 7.742 | 9.418 | 8.41 [symbol] |
| 224773_at | 5.668 | 6.94 | 7.344 | 4.953 | 7.035 | 7.352 | 6.47 [symbol] |
| 224774_s_at | 3.907 | 6.432 | 6.166 | 5.262 | 5.989 | 6.48 | 5.91 [symbol] |
| 227145_at | 7.205 | 9.531 | 9.214 | 9.691 | 3.702 | 11.838 | 8.60 [symbol] |
| 227584_at | 8.8 | 10.254 | 10.798 | 8.769 | 9.367 | 10.568 | 9.56 [symbol] |
| 227812_at | 11.39 | 9.998 | 10.948 | 9.083 | 10.541 | 12.176 | 10.5 [symbol] |
| 229723_at | 5.424 | 7.084 | 6.786 | 10.798 | 7.081 | 3.21 | 5.29 [symbol] |
| 232024_at | 9.673 | 10.09 | 9.763 | 10.696 | 9.891 | 6.523 | 8.29 [symbol] |
| 238544_at | 5.442 | 6.574 | 4.359 | 5.264 | 3.677 | 9.187 | 5.44 [symbol] |
| 242886_at | 5.974 | 8.959 | 9.284 | 6.208 | 7.589 | 9.344 | 7.33 [symbol] |
| 242986_at | 5.136 | 8.727 | 8.749 | 4.83 | 5.92 | 8.296 | 6.75 [symbol] |
| 34210_at | 7.423 | 9.382 | 9.344 | 11.944 | 10.812 | 2.854 | 9.44 [symbol] |

[symbol]

indicates data missing or illegible when filed

TABLE 14
Correlation of Patient Identity Numbers and Clinical Outcome
after MAGE Immunotherapy

| | Progressive | Stable | Mixed | | Too |
|---|---|---|---|---|---|
| | Disease | Disease | Responders | Responders | Early |
| Patient | 160 | 19 | 20 | 38 | 138 |
| ID | 30 | 77 | 67 | 136 | 91 |
| No. | 8 | 127 | 2 | 75 | 124 |
| | 109 | 65 | 11 | 120 | 150 |
| | 60 | 119 | 53 | 27 | 163 |
| | 55 | 23 | 59 | 118 | 122 |
| | 100 | 145 | 96 | | |
| | 37 | | 10 | | |
| | 52 | | 26/1 | | |
| | 81 | | | | |
| | 14 | | | | |
| | 66 | | | | |
| | 106 | | | | |
| | 133 | | | | |
| | 129 | | | | |
| | 74 | | | | |
| | 113 | | | | |
| | 36 | | | | |
| | 9 | | | | |
| | 155 | | | | |
| | 15 | | | | |
| | 5 | | | | |
| | 56 | | | | |
| | 16 | | | | |
| | 13 | | | | |
| | 98 | | | | |
| | 28 | | | | |
| | 108 | | | | |
| | 71 | | | | |
| | 145 | | | | |
| | 121 | | | | |
| | 80 | | | | |
| | 132 | | | | |
| | 3 | | | | |
| | 85 | | | | |
| | 135 | | | | |
| | 134 | | | | |
| | 82 | | | | |
| | 154 | | | | |
| | 39 | | | | |

APPENDIX A
R object of class nnet for neural network clinical outcome prediction of patients.
$n

| | | | |
|---|---|---|---|
| [1] | 489 | 2 | 1 |

$nunits

| | |
|---|---|
| [1] | 493 |

$nconn

| | | | | | | | | | | | | | | | | | | |
|---|---|---|---|---|---|---|---|---|---|---|---|---|---|---|---|---|---|---|
| [1] | 0 | 0 | 0 | 0 | 0 | 0 | 0 | 0 | 0 | 0 | 0 | 0 | 0 | 0 | 0 | 0 | 0 | 0 |
| [19] | 0 | 0 | 0 | 0 | 0 | 0 | 0 | 0 | 0 | 0 | 0 | 0 | 0 | 0 | 0 | 0 | 0 | 0 |
| [37] | 0 | 0 | 0 | 0 | 0 | 0 | 0 | 0 | 0 | 0 | 0 | 0 | 0 | 0 | 0 | 0 | 0 | 0 |
| [55] | 0 | 0 | 0 | 0 | 0 | 0 | 0 | 0 | 0 | 0 | 0 | 0 | 0 | 0 | 0 | 0 | 0 | 0 |
| [73] | 0 | 0 | 0 | 0 | 0 | 0 | 0 | 0 | 0 | 0 | 0 | 0 | 0 | 0 | 0 | 0 | 0 | 0 |
| [91] | 0 | 0 | 0 | 0 | 0 | 0 | 0 | 0 | 0 | 0 | 0 | 0 | 0 | 0 | 0 | 0 | 0 | 0 |
| [109] | 0 | 0 | 0 | 0 | 0 | 0 | 0 | 0 | 0 | 0 | 0 | 0 | 0 | 0 | 0 | 0 | 0 | 0 |
| [127] | 0 | 0 | 0 | 0 | 0 | 0 | 0 | 0 | 0 | 0 | 0 | 0 | 0 | 0 | 0 | 0 | 0 | 0 |
| [145] | 0 | 0 | 0 | 0 | 0 | 0 | 0 | 0 | 0 | 0 | 0 | 0 | 0 | 0 | 0 | 0 | 0 | 0 |
| [163] | 0 | 0 | 0 | 0 | 0 | 0 | 0 | 0 | 0 | 0 | 0 | 0 | 0 | 0 | 0 | 0 | 0 | 0 |
| [181] | 0 | 0 | 0 | 0 | 0 | 0 | 0 | 0 | 0 | 0 | 0 | 0 | 0 | 0 | 0 | 0 | 0 | 0 |
| [199] | 0 | 0 | 0 | 0 | 0 | 0 | 0 | 0 | 0 | 0 | 0 | 0 | 0 | 0 | 0 | 0 | 0 | 0 |
| [217] | 0 | 0 | 0 | 0 | 0 | 0 | 0 | 0 | 0 | 0 | 0 | 0 | 0 | 0 | 0 | 0 | 0 | 0 |
| [235] | 0 | 0 | 0 | 0 | 0 | 0 | 0 | 0 | 0 | 0 | 0 | 0 | 0 | 0 | 0 | 0 | 0 | 0 |
| [253] | 0 | 0 | 0 | 0 | 0 | 0 | 0 | 0 | 0 | 0 | 0 | 0 | 0 | 0 | 0 | 0 | 0 | 0 |
| [271] | 0 | 0 | 0 | 0 | 0 | 0 | 0 | 0 | 0 | 0 | 0 | 0 | 0 | 0 | 0 | 0 | 0 | 0 |
| [289] | 0 | 0 | 0 | 0 | 0 | 0 | 0 | 0 | 0 | 0 | 0 | 0 | 0 | 0 | 0 | 0 | 0 | 0 |
| [307] | 0 | 0 | 0 | 0 | 0 | 0 | 0 | 0 | 0 | 0 | 0 | 0 | 0 | 0 | 0 | 0 | 0 | 0 |
| [325] | 0 | 0 | 0 | 0 | 0 | 0 | 0 | 0 | 0 | 0 | 0 | 0 | 0 | 0 | 0 | 0 | 0 | 0 |
| [343] | 0 | 0 | 0 | 0 | 0 | 0 | 0 | 0 | 0 | 0 | 0 | 0 | 0 | 0 | 0 | 0 | 0 | 0 |
| [361] | 0 | 0 | 0 | 0 | 0 | 0 | 0 | 0 | 0 | 0 | 0 | 0 | 0 | 0 | 0 | 0 | 0 | 0 |
| [379] | 0 | 0 | 0 | 0 | 0 | 0 | 0 | 0 | 0 | 0 | 0 | 0 | 0 | 0 | 0 | 0 | 0 | 0 |
| [397] | 0 | 0 | 0 | 0 | 0 | 0 | 0 | 0 | 0 | 0 | 0 | 0 | 0 | 0 | 0 | 0 | 0 | 0 |
| [415] | 0 | 0 | 0 | 0 | 0 | 0 | 0 | 0 | 0 | 0 | 0 | 0 | 0 | 0 | 0 | 0 | 0 | 0 |
| [433] | 0 | 0 | 0 | 0 | 0 | 0 | 0 | 0 | 0 | 0 | 0 | 0 | 0 | 0 | 0 | 0 | 0 | 0 |
| [451] | 0 | 0 | 0 | 0 | 0 | 0 | 0 | 0 | 0 | 0 | 0 | 0 | 0 | 0 | 0 | 0 | 0 | 0 |
| [469] | 0 | 0 | 0 | 0 | 0 | 0 | 0 | 0 | 0 | 0 | 0 | 0 | 0 | 0 | 0 | 0 | 0 | 0 |
| [487] | 0 | 0 | 0 | 0 | 0 | 490 | 980 | 983 | | | | | | | | | | |

$conn

| | | | | | | | | | | | | | | | | | | |
|---|---|---|---|---|---|---|---|---|---|---|---|---|---|---|---|---|---|---|
| [1] | 0 | 1 | 2 | 3 | 4 | 5 | 6 | 7 | 8 | 9 | 10 | 11 | 12 | 13 | 14 | 15 | 16 | 17 |
| [19] | 18 | 19 | 20 | 21 | 22 | 23 | 24 | 25 | 26 | 27 | 28 | 29 | 30 | 31 | 32 | 33 | 34 | 35 |
| [37] | 36 | 37 | 38 | 39 | 40 | 41 | 42 | 43 | 44 | 45 | 46 | 47 | 48 | 49 | 50 | 51 | 52 | 53 |
| [55] | 54 | 55 | 56 | 57 | 58 | 59 | 60 | 61 | 62 | 63 | 64 | 65 | 66 | 67 | 68 | 69 | 70 | 71 |
| [73] | 72 | 73 | 74 | 75 | 76 | 77 | 78 | 79 | 80 | 81 | 82 | 83 | 84 | 85 | 86 | 87 | 88 | 89 |
| [91] | 90 | 91 | 92 | 93 | 94 | 95 | 96 | 97 | 98 | 99 | 100 | 101 | 102 | 103 | 104 | 105 | 106 | 107 |
| [109] | 108 | 109 | 110 | 111 | 112 | 113 | 114 | 115 | 116 | 117 | 118 | 119 | 120 | 121 | 122 | 123 | 124 | 125 |
| [127] | 126 | 127 | 128 | 129 | 130 | 131 | 132 | 133 | 134 | 135 | 136 | 137 | 138 | 139 | 140 | 141 | 142 | 143 |
| [145] | 144 | 145 | 146 | 147 | 148 | 149 | 150 | 151 | 152 | 153 | 154 | 155 | 156 | 157 | 158 | 159 | 160 | 161 |
| [163] | 162 | 163 | 164 | 165 | 166 | 167 | 168 | 169 | 170 | 171 | 172 | 173 | 174 | 175 | 176 | 177 | 178 | 179 |
| [181] | 180 | 181 | 182 | 183 | 184 | 185 | 186 | 187 | 188 | 189 | 190 | 191 | 192 | 193 | 194 | 195 | 196 | 197 |
| [199] | 198 | 199 | 200 | 201 | 202 | 203 | 204 | 205 | 206 | 207 | 208 | 209 | 210 | 211 | 212 | 213 | 214 | 215 |
| [217] | 216 | 217 | 218 | 219 | 220 | 221 | 222 | 223 | 224 | 225 | 226 | 227 | 228 | 229 | 230 | 231 | 232 | 233 |
| [235] | 234 | 235 | 236 | 237 | 238 | 239 | 240 | 241 | 242 | 243 | 244 | 245 | 246 | 247 | 248 | 249 | 250 | 251 |
| [253] | 252 | 253 | 254 | 255 | 256 | 257 | 258 | 259 | 260 | 261 | 262 | 263 | 264 | 265 | 266 | 267 | 268 | 269 |
| [271] | 270 | 271 | 272 | 273 | 274 | 275 | 276 | 277 | 278 | 279 | 280 | 281 | 282 | 283 | 284 | 285 | 286 | 287 |
| [289] | 288 | 289 | 290 | 291 | 292 | 293 | 294 | 295 | 296 | 297 | 298 | 299 | 300 | 301 | 302 | 303 | 304 | 305 |
| [307] | 306 | 307 | 308 | 309 | 310 | 311 | 312 | 313 | 314 | 315 | 316 | 317 | 318 | 319 | 320 | 321 | 322 | 323 |
| [325] | 324 | 325 | 326 | 327 | 328 | 329 | 330 | 331 | 332 | 333 | 334 | 335 | 336 | 337 | 338 | 339 | 340 | 341 |
| [343] | 342 | 343 | 344 | 345 | 346 | 347 | 348 | 349 | 350 | 351 | 352 | 353 | 354 | 355 | 356 | 357 | 358 | 359 |
| [361] | 360 | 361 | 362 | 363 | 364 | 365 | 366 | 367 | 368 | 369 | 370 | 371 | 372 | 373 | 374 | 375 | 376 | 377 |
| [379] | 378 | 379 | 380 | 381 | 382 | 383 | 384 | 385 | 386 | 387 | 388 | 389 | 390 | 391 | 392 | 393 | 394 | 395 |
| [397] | 396 | 397 | 398 | 399 | 400 | 401 | 402 | 403 | 404 | 405 | 406 | 407 | 408 | 409 | 410 | 411 | 412 | 413 |
| [415] | 414 | 415 | 416 | 417 | 418 | 419 | 420 | 421 | 422 | 423 | 424 | 425 | 426 | 427 | 428 | 429 | 430 | 431 |
| [433] | 432 | 433 | 434 | 435 | 436 | 437 | 438 | 439 | 440 | 441 | 442 | 443 | 444 | 445 | 446 | 447 | 448 | 449 |
| [451] | 450 | 451 | 452 | 453 | 454 | 455 | 456 | 457 | 458 | 459 | 460 | 461 | 462 | 463 | 464 | 465 | 466 | 467 |
| [469] | 468 | 469 | 470 | 471 | 472 | 473 | 474 | 475 | 476 | 477 | 478 | 479 | 480 | 481 | 482 | 483 | 484 | 485 |
| [487] | 486 | 487 | 488 | 489 | 0 | 1 | 2 | 3 | 4 | 5 | 6 | 7 | 8 | 9 | 10 | 11 | 12 | 13 |
| [505] | 14 | 15 | 16 | 17 | 18 | 19 | 20 | 21 | 22 | 23 | 24 | 25 | 26 | 27 | 28 | 29 | 30 | 31 |
| [523] | 32 | 33 | 34 | 35 | 36 | 37 | 38 | 39 | 40 | 41 | 42 | 43 | 44 | 45 | 46 | 47 | 48 | 49 |
| [541] | 50 | 51 | 52 | 53 | 54 | 55 | 56 | 57 | 58 | 59 | 60 | 61 | 62 | 63 | 64 | 65 | 66 | 67 |
| [559] | 68 | 69 | 70 | 71 | 72 | 73 | 74 | 75 | 76 | 77 | 78 | 79 | 80 | 81 | 82 | 83 | 84 | 85 |
| [577] | 86 | 87 | 88 | 89 | 90 | 91 | 92 | 93 | 94 | 95 | 96 | 97 | 98 | 99 | 100 | 101 | 102 | 103 |
| [595] | 104 | 105 | 106 | 107 | 108 | 109 | 110 | 111 | 112 | 113 | 114 | 115 | 116 | 117 | 118 | 119 | 120 | 121 |
| [613] | 122 | 123 | 124 | 125 | 126 | 127 | 128 | 129 | 130 | 131 | 132 | 133 | 134 | 135 | 136 | 137 | 138 | 139 |
| [631] | 140 | 141 | 142 | 143 | 144 | 145 | 146 | 147 | 148 | 149 | 150 | 151 | 152 | 153 | 154 | 155 | 156 | 157 |
| [649] | 158 | 159 | 160 | 161 | 162 | 163 | 164 | 165 | 166 | 167 | 168 | 169 | 170 | 171 | 172 | 173 | 174 | 175 |
| [667] | 176 | 177 | 178 | 179 | 180 | 181 | 182 | 183 | 184 | 185 | 186 | 187 | 188 | 189 | 190 | 191 | 192 | 193 |
| [685] | 194 | 195 | 196 | 197 | 198 | 199 | 200 | 201 | 202 | 203 | 204 | 205 | 206 | 207 | 208 | 209 | 210 | 211 |
| [703] | 212 | 213 | 214 | 215 | 216 | 217 | 218 | 219 | 220 | 221 | 222 | 223 | 224 | 225 | 226 | 227 | 228 | 229 |
| [721] | 230 | 231 | 232 | 233 | 234 | 235 | 236 | 237 | 238 | 239 | 240 | 241 | 242 | 243 | 244 | 245 | 246 | 247 |
| [739] | 248 | 249 | 250 | 251 | 252 | 253 | 254 | 255 | 256 | 257 | 258 | 259 | 260 | 261 | 262 | 263 | 264 | 265 |
| [757] | 266 | 267 | 268 | 269 | 270 | 271 | 272 | 273 | 274 | 275 | 276 | 277 | 278 | 279 | 280 | 281 | 282 | 283 |
| [775] | 284 | 285 | 286 | 287 | 288 | 289 | 290 | 291 | 292 | 293 | 294 | 295 | 296 | 297 | 298 | 299 | 300 | 301 |
| [793] | 302 | 303 | 304 | 305 | 306 | 307 | 308 | 309 | 310 | 311 | 312 | 313 | 314 | 315 | 316 | 317 | 318 | 319 |
| [811] | 320 | 321 | 322 | 323 | 324 | 325 | 326 | 327 | 328 | 329 | 330 | 331 | 332 | 333 | 334 | 335 | 336 | 337 |
| [829] | 338 | 339 | 340 | 341 | 342 | 343 | 344 | 345 | 346 | 347 | 348 | 349 | 350 | 351 | 352 | 353 | 354 | 355 |
| [847] | 356 | 357 | 358 | 359 | 360 | 361 | 362 | 363 | 364 | 365 | 366 | 367 | 368 | 369 | 370 | 371 | 372 | 373 |
| [865] | 374 | 375 | 376 | 377 | 378 | 379 | 380 | 381 | 382 | 383 | 384 | 385 | 386 | 387 | 388 | 389 | 390 | 391 |
| [883] | 392 | 393 | 394 | 395 | 396 | 397 | 398 | 399 | 400 | 401 | 402 | 403 | 404 | 405 | 406 | 407 | 408 | 409 |
| [901] | 410 | 411 | 412 | 413 | 414 | 415 | 416 | 417 | 418 | 419 | 420 | 421 | 422 | 423 | 424 | 425 | 426 | 427 |
| [919] | 428 | 429 | 430 | 431 | 432 | 433 | 434 | 435 | 436 | 437 | 438 | 439 | 440 | 441 | 442 | 443 | 444 | 445 |
| [937] | 446 | 447 | 448 | 449 | 450 | 451 | 452 | 453 | 454 | 455 | 456 | 457 | 458 | 459 | 460 | 461 | 462 | 463 |
| [955] | 464 | 465 | 466 | 467 | 468 | 469 | 470 | 471 | 472 | 473 | 474 | 475 | 476 | 477 | 478 | 479 | 480 | 481 |
| [973] | 482 | 483 | 484 | 485 | 486 | 487 | 488 | 489 | 0 | 490 | 491 | | | | | | | |

$nsunits

| | |
|---|---|
| [1] | 493 |

$decay

| | |
|---|---|
| [1] | 0 |

$entropy

| | |
|---|---|
| [1] | TRUE |

$softmax

| | |
|---|---|
| [1] | FALSE |

$censored

| | |
|---|---|
| [1] | FALSE |

$value

| | |
|---|---|
| [1] | 9.930496e−05 |

$wts

| | | | | | |
|---|---|---|---|---|---|
| [1] | −7.822649e−01 | −2.605320e−01 | −3.445762e−01 | 1.193835e+00 | 7.980130e−01 |
| [6] | −1.454517e−01 | −3.852555e−01 | −6.038561e−01 | 3.628980e−01 | 1.214178e−01 |
| [11] | 6.302635e−01 | 1.163077e+00 | −1.348927e+00 | 4.313002e−01 | −3.439181e−01 |
| [16] | 1.354545e+00 | −5.330816e−01 | 9.006249e−04 | −3.553966e−01 | −7.807184e−01 |
| [21] | −2.792791e−01 | −3.170041e−01 | −7.652292e−01 | 7.708915e−02 | −8.561736e−01 |
| [26] | 5.142947e−01 | −2.484503e−01 | 9.726332e−01 | 1.778460e−01 | −2.21F09e+00 |
| [31] | 1.533794e−01 | −9.241165e−01 | −1.623889e+00 | −1.673846e−01 | −3.942404e−01 |
| [36] | 4.839469e−02 | −6.068823e−01 | −7.231734e−01 | 2.776973e−01 | 9.486888e−01 |
| [41] | −3.606490e−01 | −9.814695e−02 | −4.978396e−01 | 6.743036e−01 | −6.715348e−01 |
| [46] | 3.743046e−01 | 1.127027e+00 | 2.424162e−01 | −2.472467e−01 | 5.103072e−01 |
| [51] | −2.249262e−01 | 9.216276e−02 | −7.811338e−02 | 6.543984e−02 | −9.300402e−01 |
| [56] | −1.723861e−01 | 8.450912e−01 | −1.112637e−01 | −1.340432e+00 | −4.579130e−01 |
| [61] | −2.000303e−01 | −6.724820e−01 | 3.185803e−02 | −8.755603e−01 | −1.432133e−01 |
| [66] | 1.734187e−01 | −9.095931e−01 | −5.779322e−01 | −6.239654e−01 | 5.350169e−01 |
| [71] | 2.660352e−02 | 7.575969e−01 | −5.857182e−01 | 7.047356e−01 | 2.619782e−01 |
| [76] | 7.365027e−02 | 1.085127e+00 | −6.649458e−01 | 1.526824e−02 | 9.521713e−01 |
| [81] | 2.953514e−01 | −5.496499e−02 | −7.349885e−01 | 4.766398e−01 | −4.030412e−01 |
| [86] | 8.509923e−01 | −2.896527e−02 | −4.922158e−02 | −1.075633e+00 | 2.852051e−01 |
| [91] | −2.040135e−01 | 7.087471e−01 | −7.626716e−01 | 4.932172e−02 | −1.068290e+00 |
| [96] | 4.525142e−01 | −3.426858e−01 | −5.564159e−01 | 4.206087e−01 | 3.702788e−01 |
| [101] | −8.482293e−01 | 2.445519e−01 | −2.423795e−01 | −7.502546e−01 | −2.448160e−01 |
| [106] | −1.029814e−01 | −4.534969e−01 | −5.680071e−01 | −2.278669e−01 | 4.914741e−01 |
| [111] | −2.408882e−02 | −1.122049e+00 | 1.637105e+00 | −8.674111e−01 | −3.993925e−01 |
| [116] | 6.766219e−01 | −2.590604e−01 | −1.128037e−01 | −5.454970e−01 | 3.117549e−01 |
| [121] | 6.807225e−01 | 1.767702e+00 | 1.749150e+00 | 1.576723e+00 | 1.000372e+00 |
| [126] | −3.135498e−01 | −3.461115e−01 | −1.577623e−01 | −3.688724e−03 | −5.169024e−01 |
| [131] | 5.209675e−01 | 6.239938e−01 | −6.562843e−01 | 3.318594e−01 | 1.073695e+00 |
| [136] | 3.852917e−01 | 7.445331e−01 | 6.147421e−01 | 3.486045e−02 | 2.328786e−01 |
| [141] | 5.865109e−01 | 8.789772e−01 | 2.735659e−01 | 2.179924e−01 | 6.546454e−01 |
| [146] | −7.425786e−02 | 2.332181e−01 | 2.026860e−02 | 1.920527e+00 | −2.199749e−01 |
| [151] | −2.861057e−01 | −6.113327e−01 | −7.327236e−01 | 7.630948e−01 | −6.725150e−01 |
| [156] | 8.195715e−01 | 1.354855e−01 | 7.557001e−01 | −6.311882e−01 | 4.028033e−01 |
| [161] | −1.095942e−01 | 2.675720e−02 | 9.191105e−02 | −5.412179e−01 | −4.676564e−01 |
| [166] | −7.538073e−02 | −8.129017e−01 | −4.018241e−01 | −2.996730e−01 | −5.965576e−01 |
| [171] | 9.892726e−01 | 5.105776e−01 | 1.052146e+00 | −2.957608e−01 | −4.917327e−01 |
| [176] | 2.512329e−01 | −1.016831e−01 | 1.099494e−02 | −6.844977e−01 | 5.180294e−01 |
| [181] | 5.463099e−02 | −5.062577e−01 | 5.281003e−01 | −5.333424e−01 | 9.727653e−01 |
| [186] | −8.865765e−01 | 5.137428e−01 | −1.480626e−01 | −1.719326e−01 | 7.033034e−01 |
| [191] | 5.484301e−01 | 4.860635e−01 | −4.432808e−01 | 6.295423e−01 | 6.296338e−01 |
| [196] | 3.257622e−01 | 5.230624e−01 | 3.651660e−01 | 7.889078e−01 | −1.381056e−02 |
| [201] | 1.092450e−01 | 9.362120e−01 | −9.812814e−02 | 5.535681e−01 | 1.927306e−01 |
| [206] | 4.132113e−01 | 2.483396e−01 | −4.509187e−01 | −5.662541e−01 | 6.316726e−01 |
| [211] | −1.081482e+00 | −2.642237e−01 | 5.756106e−01 | 7.014808e−01 | 8.240274e−02 |
| [216] | 1.157531e−01 | 2.011787e−01 | 6.522527e−01 | 1.072262e−01 | 7.789481e−01 |
| [221] | −3.298577e−02 | −2.177002e+00 | −5.493206e−01 | −3.840635e−01 | −5.162878e−01 |
| [226] | −3.685617e−01 | 2.763670e−01 | −4.768905e−01 | 2.604606e−01 | 3.470977e−01 |
| [231] | 1.882645e−01 | −1.529664e−01 | −1.141422e+00 | −1.192761e+00 | 9.653415e−01 |
| [236] | −5.616165e−02 | 7.073548e−01 | 1.663138e−01 | −1.724516e−02 | 5.466189e−01 |
| [241] | 8.215863e−02 | −2.863819e−01 | −7.682638e−01 | 6.907757e−01 | 2.953517e−01 |
| [246] | −8.597825e−02 | −1.237122e−01 | −1.141088e−00 | 7.686872e−01 | 1.278818e−01 |
| [251] | 1.363887e−01 | 2.012764e−01 | −2.660433e−01 | −2.356637e−01 | −1.108908e+00 |
| [256] | 4.767741e−01 | −3.796921e−01 | −8.998346e−01 | 4.556543e−01 | −1.754178e−01 |
| [261] | 1.102156e+00 | −6.089921e−01 | 4.790648e−01 | −1.378465e+00 | −7.794537e−01 |
| [266] | −4.038758e−01 | 1.344525e+00 | −4.043755e−01 | −4.395272e−01 | 6.362795e−01 |
| [271] | 1.834397e−01 | 8.358359e−03 | −3.857196e−01 | 7.268706e−01 | −7.168161e−01 |
| [276] | 4.306795e−01 | 7.706614e−01 | 6.513772e−02 | −2.245757e−01 | −1.063308e+00 |
| [281] | 1.798277e−01 | −1.752740e−01 | 2.278172e−01 | 2.965010e−01 | 5.452244e−01 |
| [286] | 1.543681e−01 | 1.661905e−01 | −2.996078e−01 | 1.187087e−01 | 1.133628e−01 |
| [291] | 9.718658e−01 | 4.520898e−01 | 9.167133e−01 | −3.433412e−01 | 3.579116e−01 |
| [296] | 6.023363e−03 | 2.989546e−02 | −4.734359e−01 | 5.843214e−01 | −3.799736e−01 |
| [301] | 5.842987e−01 | 1.525014e+00 | 1.118956e−01 | 4.651440e−01 | 5.483678e−01 |
| [306] | −3.747111e−02 | −6.338612e−01 | 1.383897e−01 | 1.423111e−01 | 8.514850e−01 |
| [311] | 6.421267e−01 | 1.666450e−01 | −2.689546e−01 | −1.752850e−01 | 6.560046e−02 |
| [316] | 3.302397e−01 | 5.434177e−02 | 7.720145e−01 | −2.185973e−01 | −1.400281e−01 |
| [321] | −3.805316e−01 | 5.617241e−01 | −7.327657e−01 | −9.889267e−01 | −1.877597e−01 |
| [326] | −4.126207e−01 | −1.432952e+00 | −2.641545e−01 | −1.279892e−01 | 3.308045e−01 |
| [331] | 2.947318e−01 | 3.080860e−01 | 4.368988e−01 | −7.796344e−01 | 6.472218e−01 |
| [336] | 3.216706e−01 | 3.220898e−01 | −7.642426e−01 | 7.559186e−01 | −7.224366e−02 |
| [341] | 8.057934e−01 | −8.280142e−01 | 5.811389e−01 | 8.082749e−01 | −1.739542e−01 |
| [346] | 1.878581e−01 | −9.195490e−01 | 4.122718e−01 | 4.458733e−01 | −5.164353e−01 |
| [351] | −2.987246e−01 | −2.953580e−02 | −1.027130e−00 | 1.380701e+00 | 1.015581e+00 |
| [356] | 7.511154e−01 | −1.295990e−01 | −1.289274e−01 | 2.716158e−01 | 4.902457e−01 |
| [361] | −8.143006e−01 | 2.466899e−01 | −7.051082e−01 | −1.421216e+00 | 7.574016e−01 |
| [366] | 1.062493e+00 | 3.120192e−01 | −8.555016e−01 | −2.710965e−01 | 5.270817e−01 |
| [371] | −2.772449e−02 | −1.164226e+00 | −5.875827e−01 | 4.967017e−01 | −9.446788e−02 |
| [376] | 4.286175e−01 | 6.268223e−01 | −3.435110e−01 | 1.025191e+00 | −6.801600e−01 |
| [381] | −3.224292e−02 | −3.193044e−01 | −3.950073e−01 | −6.430585e−01 | −4.542940e−01 |
| [386] | −8.485502e−01 | 7.760473e−01 | 3.202145e−01 | 6.633763e−01 | −5.801942e−01 |
| [391] | 1.306814e−01 | 1.299666e−01 | 3.046116e−01 | −1.684422e−01 | −6.295904e−01 |
| [396] | 1.565646e−01 | −1.529663e−01 | −1.148021e−02 | −4.255510e−01 | −4.986614e−01 |
| [401] | −2.557943e−01 | −6.037794e−01 | −1.125332e−01 | 7.177677e−01 | −5.615997e−01 |
| [406] | 3.587169e−02 | 8.140494e−01 | −6.603336e−01 | 5.388208e−02 | 2.157824e−01 |
| [411] | 8.007642e−01 | 1.088418e+00 | 7.859298e−01 | −5.836605e−01 | −3.394254e−01 |
| [416] | −7.943000e−01 | 5.089051e−01 | 2.668172e−01 | −9.942403e−01 | −6.977250e−01 |
| [421] | 5.418000e−02 | 9.294052e−01 | −5.541002e−01 | −2.663888e−01 | 1.582470e−01 |
| [426] | −9.034341e−01 | 1.796377e−01 | −1.666522e+00 | −6.447704e−01 | −1.460522e−01 |
| [431] | 4.064656e−01 | 4.535141e−01 | −8.493838e−02 | 4.404440e−01 | −6.813737e−01 |
| [436] | 4.246934e−01 | 4.738893e−01 | 5.388799e−01 | 5.893005e−02 | 8.046329e−01 |
| [441] | −1.312353e−01 | 6.065425e−01 | 4.344549e−01 | −1.432887e−01 | 6.266481e−01 |
| [446] | 2.491106e−01 | 5.628406e−01 | 7.156338e−01 | −3.341744e−01 | −7.083861e−01 |
| [451] | −3.816457e−01 | 7.333444e−01 | 8.614763e−01 | 1.158857e+00 | 4.593186e−01 |
| [456] | −1.059777e−01 | −2.820036e−01 | 5.517071e−01 | −3.863232e−01 | −1.729483e+00 |
| [461] | −1.887161e−01 | 3.810323e−01 | 7.033161e−02 | 4.999075e−01 | 1.066108e−01 |
| [466] | −1.682395e−01 | 2.324000e−02 | −1.079890e−01 | −9.324107e−02 | −1.789810e−02 |
| [471] | −1.151141e+00 | 1.302142e−01 | −4.902189e−01 | −9.327821e−03 | 2.853960e−02 |
| [476] | 3.681996e−01 | −5.675777e−02 | −1.121354e+00 | 9.055091e−01 | −3.077752e−01 |
| [481] | 1.150414e−01 | 2.903319e−01 | −1.978837e−01 | −2.513171e−01 | −6.335767e−01 |
| [486] | 1.883908e−01 | 7.978107e−01 | 5.193154e−01 | 4.594379e−01 | −5.630128e−01 |
| [491] | −2.558691e−01 | 1.399098e−01 | 2.716841e−01 | −1.029870e+00 | −3.337377e−01 |
| [496] | −1.639137e−01 | 1.084488e+00 | −2.009724e−01 | 2.988744e−02 | −9.373952e−03 |
| [501] | 7.188562e−02 | 1.568801e−01 | 1.110666e+00 | −6.429716e−01 | −9.851071e−02 |
| [506] | −3.954610e−01 | 6.241091e−01 | 4.466790e−01 | −3.418489e−01 | 1.129400e+00 |
| [511] | 7.873880e−02 | 3.685607e−01 | 1.312878e+00 | 3.921632e−01 | −9.862630e−01 |
| [516] | 7.303611e−01 | 8.164471e−01 | −7.706864e−01 | 1.701961e−01 | 9.772512e−01 |
| [521] | 3.345849e−01 | 5.069341e−01 | 9.739133e−01 | 1.608824e+00 | 1.145627e+00 |
| [526] | 5.074447e−01 | 2.034436e−01 | 1.470176e−01 | −1.600484e−01 | −2.150473e−01 |
| [531] | 1.377926e−01 | 6.227566e−01 | 4.094106e−01 | 4.259897e−01 | 1.865411e−01 |
| [536] | −4.727419e−01 | −2.291728e−01 | 1.015483e+00 | 1.073489e+00 | −2.136211e−01 |
| [541] | −8.307180e−01 | −2.981026e−01 | 1.194316e+00 | −1.318526e+00 | 1.129897e+00 |
| [546] | −6.792031e−02 | −1.136511e−01 | 1.129797e+00 | 1.283430e+00 | 1.225369e+00 |
| [551] | −4.935841e−01 | −5.978041e−01 | 6.994883e−01 | −5.000395e−01 | −5.917541e−02 |
| [556] | 8.643770e−01 | −8.313903e−02 | −7.645437e−01 | 4.585311e−01 | 2.760396e−01 |
| [561] | −9.048995e−01 | 2.270971e−01 | −5.491731e−01 | 1.485625e−01 | 5.379235e−01 |
| [566] | 2.400513e−01 | −1.398254e+00 | 2.149663e−01 | −9.872386e−01 | −2.349650e−01 |
| [571] | −1.134452e+00 | 1.084613e+00 | −5.258544e−01 | −2.309454e−01 | −1.118639e+00 |
| [576] | −6.754324e−01 | −1.105271e+00 | −3.729548e−01 | 4.167448e−01 | −6.998005e−02 |
| [581] | −6.067353e−01 | −8.627442e−01 | −2.747273e−01 | −6.861895e−01 | −8.294093e−01 |
| [586] | −7.157550e−01 | −1.543888e+00 | 8.807905e−01 | 1.449671e−01 | 6.694883e−01 |
| [591] | 8.768986e−01 | −3.354555e−01 | −3.288934e−01 | −6.713428e−02 | −8.535772e−01 |
| [596] | 3.177712e−01 | 2.302577e−01 | 6.388607e−01 | −8.468490e−01 | −6.823332e−01 |
| [601] | −4.824133e−01 | −1.152830e+00 | −1.296166e−01 | 3.876039e−01 | −1.264702e−01 |
| [606] | −6.234923e−01 | 7.556986e−01 | −1.079503e+00 | 7.462365e−01 | −7.156638e−01 |
| [611] | −6.597890e−01 | −9.319472e−01 | 7.275191e−01 | −1.206812e+00 | 1.462996e−01 |
| [616] | 3.163475e−01 | −4.897914e−01 | 9.590103e−01 | 4.964603e−01 | −2.036444e−01 |
| [621] | 5.536274e−01 | −8.859709e−01 | 4.360544e−01 | −1.174303e+00 | −7.004479e−01 |
| [626] | −9.104478e−02 | 2.520933e−01 | 1.406452e−01 | −5.811733e−01 | 1.005106e+00 |
| [631] | −8.395171e−01 | 2.836866e−01 | −6.667517e−01 | 7.589487e−01 | 9.250607e−01 |
| [636] | 2.226882e−01 | −9.617627e−01 | 7.764753e−01 | −2.592188e−01 | −3.870735e−01 |
| [641] | 3.652488e−01 | −2.089410e+00 | −8.639070e−01 | 6.041561e−02 | −1.249116e−01 |
| [646] | −1.589578e−01 | −9.288991e−01 | −1.438925e−01 | −1.584532e−01 | 4.032568e−01 |
| [651] | −4.542904e−01 | 4.196369e−01 | 3.925464e−02 | −7.026149e−01 | −4.894067e−01 |
| [656] | 1.472926e−01 | 5.125774e−01 | −7.921361e−01 | −6.512886e−01 | 6.268859e−01 |
| [661] | −8.082803e−01 | −1.230153e−01 | −1.259874e+00 | −7.360178e−01 | −4.800681e−01 |
| [666] | −9.807421e−02 | 3.650259e−01 | −1.110299e+00 | −6.785503e−02 | 8.200076e−02 |
| [671] | −7.377264e−01 | 9.628769e−01 | −6.621068e−01 | −1.064977e+00 | 1.635347e−01 |
| [676] | 3.436851e−01 | −4.931667e−01 | 1.110185e+00 | −5.205865e−01 | 6.614455e−01 |
| [681] | −2.341954e−01 | −3.812965e−01 | −3.107048e−01 | −5.694353e−01 | 2.589693e−01 |
| [686] | −5.235213e−01 | 1.573854e−01 | −4.416297e−01 | 2.271200e−01 | −9.274423e−01 |
| [691] | 2.991815e−01 | −2.355672e−01 | −6.642878e−01 | −2.300048e−01 | 3.206771e−01 |
| [696] | −2.170961e+00 | −5.213502e−01 | −1.148505e+00 | −6.783888e−01 | −4.686891e−04 |
| [701] | 7.177564e−01 | −3.765768e−02 | −1.415048e−02 | −2.595464e−01 | 2.996030e−01 |
| [706] | −1.233281e−01 | −3.676282e−01 | −3.679718e−01 | −9.210808e−01 | 4.052164e−01 |
| [711] | 4.585852e−02 | 1.312361e+00 | 5.048614e−01 | −1.809305e+00 | −1.721751e+00 |
| [716] | −1.669053e+00 | 1.401519e−01 | −8.288580e−01 | 1.470533e−01 | −1.315543e+00 |
| [721] | 6.430333e−02 | −7.649410e−01 | 9.157078e−01 | 3.640731e−01 | −1.306945e−02 |
| [726] | 9.203552e−02 | −6.416202e−01 | −6.434372e−01 | −7.309035e−01 | 6.760630e−01 |
| [731] | 5.793554e−01 | 3.266417e−01 | −5.467879e−01 | 1.875603e−02 | 6.823768e−01 |
| [736] | 6.605321e−01 | −2.192790e−01 | 6.458484e−01 | 1.256230e−01 | −3.446176e−01 |
| [741] | −7.349580e−01 | −3.794139e−01 | −5.742886e−01 | −7.069700e−01 | 2.605697e−01 |
| [746] | −2.759157e+00 | −3.153237e−01 | 6.614816e−01 | −1.399808e+00 | 2.537040e−01 |
| [751] | −2.566458e−01 | −9.300803e−01 | −3.258002e−01 | 1.847266e+00 | −8.318504e−01 |
| [756] | 8.032399e−01 | −7.391342e−02 | −1.458280e−01 | 1.475066e−01 | −2.016509e−01 |
| [761] | −1.116992e+00 | 2.565951e−01 | −1.417568e+00 | −4.075312e−01 | −4.624426e−01 |
| [766] | −3.892320e−01 | 9.233114e−02 | 8.039994e−01 | 1.708351e−01 | 3.140843e−01 |
| [771] | 1.558756e−01 | 4.926293e−01 | −1.257969e+00 | −7.845624e−01 | 1.556651e−01 |
| [776] | −1.152795e+00 | −1.463677e+00 | −8.108185e−01 | −8.317870e−02 | −1.456545e−01 |
| [781] | 6.954507e−01 | −8.047871e−01 | −8.898140e−01 | −1.254371e+00 | −6.563698e−01 |
| [786] | 3.967049e−01 | 9.335104e−01 | 1.299258e−02 | −2.453240e−01 | −5.841076e−01 |
| [791] | −1.658272e−01 | −1.326827e+00 | 8.844022e−01 | −6.606743e−01 | 4.137678e−01 |
| [796] | 7.360620e−02 | −1.241175e+00 | 1.066117e+00 | −1.086576e−01 | 7.812635e−01 |
| [801] | −5.793264e−01 | 3.664636e−02 | 5.461605e−01 | −1.468411e−01 | −1.200992e+00 |
| [806] | 7.664790e−02 | −9.648972e−01 | 8.464096e−02 | 8.781079e−01 | −1.329100e−01 |
| [811] | −3.140853e−01 | −3.603208e−01 | 5.330240e−01 | 9.776989e−01 | 1.754023e−01 |
| [816] | −3.932540e−02 | 1.381857e+00 | 4.967358e−01 | 2.445305e−01 | 2.033007e−02 |
| [821] | −1.660898e+00 | −3.826148e−01 | −1.350548e−01 | 2.055229e−01 | −4.826541e−01 |
| [826] | −3.437897e−02 | −1.131522e+00 | −2.924096e−01 | 1.078663e+00 | −3.618922e−01 |
| [831] | −3.795694e−01 | 7.062471e−01 | 1.818627e−01 | 3.360420e−02 | −5.509515e−01 |
| [836] | 2.820342e−01 | −5.169129e−01 | −7.298595e−01 | 6.831575e−02 | −4.718518e−01 |
| [841] | −1.177961e−01 | 3.261549e−01 | 9.243626e−01 | −9.539287e−01 | −8.574709e−01 |
| [846] | 8.404623e−01 | −1.477904e+00 | 7.161732e−01 | −7.213226e−02 | −1.072695e+00 |
| [851] | 1.099245e+00 | 3.907082e−02 | 4.707593e−01 | −1.037259e+00 | −6.337726e−01 |
| [856] | −6.489972e−01 | −8.105587e−01 | −1.132535e−01 | 5.618367e−01 | 1.120736e+00 |
| [861] | −1.589420e−01 | −4.736779e−01 | −3.492589e−01 | −5.292510e−01 | 7.552729e−02 |
| [866] | 6.245302e−01 | −1.599877e+00 | 1.961020e+00 | −5.986310e−01 | 7.907619e−01 |
| [871] | 9.258619e−01 | 4.602164e−01 | −4.456243e−01 | 2.256970e−01 | 1.180578e+00 |
| [876] | −2.202471e−01 | −1.595068e+00 | −7.650998e−01 | 1.068712e−01 | 3.886734e−03 |
| [881] | 3.158598e−02 | −7.733971e−01 | 1.905452e−01 | −6.009153e−01 | 2.211837e−01 |
| [886] | −5.134809e−02 | −8.393086e−01 | −1.087550e+00 | −3.173469e−01 | −1.101492e+00 |
| [891] | 4.972408e−01 | 1.799967e−01 | −2.447001e−01 | −8.741257e−01 | 2.992654e−01 |
| [896] | −9.245988e−01 | 9.044085e−02 | 9.133224e−01 | 7.446155e−01 | −7.193918e−01 |
| [901] | 9.205514e−02 | −1.402019e+00 | 4.976776e−01 | −6.959568e−01 | 4.945441e−01 |
| [906] | 1.286614e−01 | −8.543565e−01 | −3.603456e−02 | 4.664471e−01 | −7.580873e−03 |
| [911] | −3.060679e−01 | −6.700308e−01 | 4.941296e−04 | −8.978372e−01 | −6.954622e−01 |
| [916] | −3.736479e−01 | 1.754756e−02 | 1.567201e+00 | 8.573695e−01 | 3.966870e−01 |
| [921] | −2.215584e−02 | −1.169949e−01 | 4.257213e−01 | −2.346597e−01 | 7.421774e−01 |
| [926] | −5.966420e−01 | 5.751260e−02 | −2.472787e−01 | 2.334021e−01 | 1.300946e+00 |
| [931] | 2.341594e−01 | 1.049020e+00 | −3.269379e−01 | −1.269805e−01 | −6.880527e−01 |
| [936] | −3.229197e−01 | 7.076868e−01 | −3.403887e−01 | 1.676770e−01 | −7.620830e−02 |
| [941] | 3.558033e−01 | 1.459493e−01 | −6.244181e−01 | 1.770295e−01 | −1.216517e−01 |
| [946] | 7.426668e−01 | −1.385593e−01 | −6.277839e−01 | −1.879113e−01 | 9.488544e−02 |
| [951] | 2.351063e−01 | 1.073347e+00 | −4.874189e−01 | 3.801474e−01 | −6.690750e−01 |
| [956] | −6.047573e−01 | −3.134005e−01 | −6.000130e−01 | −3.197655e−01 | 6.631834e−01 |
| [961] | −6.356578e−01 | −5.904780e−01 | 1.097918e+00 | 6.007569e−01 | −2.505695e+00 |
| [966] | −5.697962e−01 | 5.674896e−01 | 4.245279e−01 | −1.060281e−01 | −1.130024e−01 |
| [971] | 1.595858e+00 | 1.516974e−01 | −3.702457e−02 | −1.700967e−01 | −3.641777e−01 |
| [976] | −1.604025e−01 | −1.447675e+00 | −1.008405e+00 | 3.365701e−01 | 2.348416e−01 |
| [981] | −1.189562e+01 | 2.427842e+01 | −2.275821e+01 | | |

[,1]

$fitted.values

| | |
|---|---|
| 1 | 9.999958e−01 |
| 2 | 0.000000e+00 |
| 3 | 0.000000e+00 |
| 4 | 0.000000e+00 |
| 5 | 0.000000e+00 |
| 6 | 9.999958e−01 |
| 7 | 9.999958e−01 |
| 8 | 9.999958e−01 |
| 9 | 9.999958e−01 |
| 10 | 9.999958e−01 |
| 11 | 9.999958e−01 |
| 12 | 0.000000e+00 |
| 13 | 9.999958e−01 |
| 14 | 0.000000e+00 |
| 15 | 0.000000e+00 |
| 16 | 0.000000e+00 |
| 17 | 9.999958e−01 |
| 18 | 6.820198e−06 |
| 19 | 6.825022e−06 |
| 20 | 3.118923e−05 |
| 21 | 0.000000e+00 |
| 22 | 0.000000e+00 |
| 23 | 9.999958e−01 |
| 24 | 0.000000e+00 |
| 25 | 0.000000e+00 |
| 26 | 9.999958e−01 |
| 27 | 0.000000e+00 |
| 28 | 9.999958e−01 |
| 29 | 9.999958e−01 |
| 30 | 0.000000e+00 |
| 31 | 0.000000e+00 |

$residuals

| | |
|---|---|
| 1 | 4.189989e−06 |
| 2 | 0.000000e+00 |
| 3 | 0.000000e+00 |
| 4 | 0.000000e+00 |
| 5 | 0.000000e+00 |
| 6 | 4.189989e−06 |
| 7 | 4.189989e−06 |
| 8 | 4.189989e−06 |
| 9 | 4.189989e−06 |
| 10 | 4.189989e−06 |
| 11 | 4.189989e−06 |
| 12 | 0.000000e+00 |
| 13 | 4.189989e−06 |
| 14 | 0.000000e+00 |
| 15 | 0.000000e+00 |
| 16 | 0.000000e+00 |
| 17 | 4.189989e−06 |
| 18 | −6.820198e−06 |
| 19 | −6.825022e−06 |
| 20 | −3.118923e−05 |
| 21 | 0.000000e+00 |
| 22 | 0.000000e+00 |
| 23 | 4.189989e−06 |
| 24 | 0.000000e+00 |
| 25 | 0.000000e+00 |
| 26 | 4.189989e−06 |
| 27 | 0.000000e+00 |
| 28 | 4.189989e−06 |
| 29 | 4.189989e−06 |
| 30 | 0.000000e+00 |
| 31 | 0.000000e+00 |

$lev

| | |
|---|---|
| [1] | “NR” “R” |

$call
nnet.formula(formula = f ~ .,
data = data, MaxNWts = 10000,
maxit = 250, size = 2, skip = FALSE,
rang = 0.7, decay = 0, Hess = FALSE,
trace = TRUE, abstol = 1e−04,
reltol = 1e−08, metric =
“euclidean”, subset = training)

APPENDIX B
R object of class Ida for linear discriminant analysis clinical outcome
prediction of patients.

| | |
|---|---|
| NR | R |

$prior

| | |
|---|---|
| 0.5862069 | 0.4137931 |

$counts

| | |
|---|---|
| 17 | 12 |

$scaling

| | LD1 |
|---|---|
| X1405_i_at | 5.944209e−03 |
| X1552497_a_at | −1.489159e−03 |
| X1552612_at | 1.928010e−03 |
| X1552613_s_at | −3.948620e−03 |
| X1553102_a_at | 2.035336e−02 |
| X1553132_a_at | −1.048751e−02 |
| X1553313_s_at | −3.034153e−02 |
| X1553906_s_at | −1.834813e−02 |
| X1554240_a_at | 1.365655e−02 |
| X1554966_a_at | 3.759930e−03 |
| X1555229_a_at | 8.797787e−02 |
| X1555630_a_at | −2.791031e−02 |
| X1555756_a_at | 1.654466e−02 |
| X1555759_a_at | 1.036814e−03 |
| X1555812_a_at | 1.278876e−02 |
| X1555852_at | −3.512472e−03 |
| X1556185_a_at | 3.025315e−02 |
| X1556579_s_at | 4.497852e−02 |
| X1557116_at | 4.611121e−03 |
| X1557222_at | 2.410949e−02 |
| X1558034_s_at | 2.790575e−02 |
| X1558290_a_at | −1.523617e−02 |
| X1558586_at | −4.151460e−02 |
| X1558972_s_at | 5.561221e−04 |
| X1559263_s_at | 7.463183e−03 |
| X1559425_at | 1.864440e−03 |
| X1559584_a_at | −5.820026e−03 |
| X1562031_at | 9.246741e−03 |
| X1563461_at | −2.064194e−02 |
| X1563473_at | 4.011858e−03 |
| X1565602_at | −4.884858e−02 |
| X1568736_s_at | −2.773483e−02 |
| X1568822_at | 2.099681e−03 |
| X1569942_at | 6.739294e−03 |
| X200612_s_at | −6.365853e−02 |
| X200904_at | 6.387716e−03 |
| X200905_x_at | 5.294827e−03 |
| X200953_s_at | −8.671791e−03 |
| X201010_s_at | 4.474642e−03 |
| X201137_s_at | −1.506315e−02 |
| X201220_x_at | −2.667959e−02 |
| X2071236_s_at | 1.404961e−02 |
| X201425_at | 1.554231e−02 |
| X201474_s_at | −6.107372e−03 |
| X201487_at | 2.472156e−02 |
| X201497_x_at | 7.254710e−04 |
| X201502_s_at | 5.696526e−03 |
| X201531_at | 1.148431e−02 |
| X201566_x_at | 1.794849e−02 |
| X201720_s_at | 1.570380e−03 |
| X201721_s_at | −9.613928e−03 |
| X201804_x_at | −2.053567e−02 |
| X201859_at | −1.376084e−04 |
| X201939_at | −3.369784e−02 |
| X202207_at | −1.196899e−02 |
| X202255_s_at | −4.340999e−02 |
| X202269_x_at | −1.833048e−02 |
| X202270_at | −8.686638e−03 |
| X202368_s_at | −2.233262e−02 |
| X202369_s_at | −1.278489e−02 |
| X202391_at | −5.586253e−03 |
| X202510_s_at | 1.200980e−02 |
| X202625_at | 1.449689e−02 |
| X202643_s_at | 2.057522e−02 |
| X202644_s_at | 2.044322e−02 |
| X202687_s_at | 1.939420e−03 |
| X202688_at | 8.690145e−04 |
| X202948_at | −1.822099e−02 |
| X202957_at | −1.143226e−02 |
| X203413_at | −3.803468e−02 |
| X203416_at | −1.189864e−02 |
| X203471_s_at | 1.553784e−02 |
| X203508_at | 9.486448e−03 |
| X203523_at | 5.575011e−03 |
| X203547_at | 9.218142e−03 |
| X203665_at | 3.487226e−02 |
| X203741_s_at | 2.161880e−02 |
| X203761_at | 8.568780e−03 |
| X203812_at | 4.104549e−02 |
| X203868_s_at | −3.218808e−02 |
| X203915_at | 1.356669e−02 |
| X203932_at | −1.767768e−03 |
| X204057_at | 1.199848e−02 |
| X204070_at | −8.013273e−04 |
| X204116_at | 6.190984e−03 |
| X204118_at | 1.625516e−02 |
| X204135_at | 4.612466e−03 |
| X204204_at | 3.177067e−02 |
| X204220_at | −6.573462e−03 |
| X204222_s_at | 8.871953e−03 |
| X204224_s_at | −4.997460e−03 |
| X204233_s_at | −1.586361e−02 |
| X204236_at | 5.467904e−03 |
| X204249_s_at | −1.313280e−02 |
| X204411_at | 6.579659e−03 |
| X204438_at | 2.031749e−02 |
| X204502_at | 6.392044e−03 |
| X204512_at | −1.534429e−05 |
| X204533_at | 2.287973e−02 |
| X204613_at | 5.458783e−03 |
| X204628_s_at | −3.028829e−02 |
| X204642_at | −3.867430e−03 |
| X204655_at | −5.765680e−03 |
| X204661_at | 2.636612e−02 |
| X204670_x_at | −1.182992e−02 |
| X204687_at | 3.953856e−02 |
| X204724_s_at | −1.982230e−02 |
| X204774_at | −1.596679e−02 |
| X204778_x_at | −2.436425e−02 |
| X204834_at | 4.664052e−02 |
| X204846_at | 2.224653e−02 |
| X204894_s_at | 4.442071e−03 |
| X204897_at | −2.250415e−02 |
| X204912_at | 2.155492e−03 |
| X204923_at | −9.860185e−03 |
| X205027_s_at | 3.406286e−03 |
| X205039_s_at | 1.453129e−03 |
| X205081_at | 2.008363e−02 |
| X205159_at | 4.374158e−03 |
| X205225_at | −8.687131e−03 |
| X205226_at | −4.151219e−02 |
| X205251_at | 2.071759e−02 |
| X205285_s_at | 9.469306e−03 |
| X205392_s_at | 1.236199e−02 |
| X205403_at | 1.344662e−02 |
| X205419_at | −6.256056e−03 |
| X205421_at | 1.955389e−02 |
| X205440_s_at | 2.903482e−02 |
| X205484_at | −1.026029e−02 |
| X205488_at | 2.109358e−02 |
| X205559_s_at | −2.837597e−02 |
| X205569_at | 1.407987e−02 |
| X205624_at | 2.531892e−02 |
| X205685_at | 4.266186e−02 |
| X205696_s_at | −7.143024e−03 |
| X205758_at | 9.290501e−03 |
| X205786_s_at | −7.188176e−03 |
| X205831_at | 8.970703e−03 |
| X205841_at | −1.024458e−02 |
| X205844_at | 2.406034e−02 |
| X205890_s_at | 1.526584e−02 |
| X205987_at | 5.099598e−03 |
| X206082_at | −2.365415e−02 |
| X206099_at | 4.205302e−02 |
| X206118_at | 1.153657e−02 |
| X206134_at | −4.474477e−03 |
| X206170_at | −1.785743e−02 |
| X206204_at | 1.969866e−02 |
| X206295_at | 1.528790e−02 |
| X206385_s_at | −1.655446e−02 |
| X206407_s_at | 3.592881e−02 |
| X206545_at | 1.272019e−02 |
| X206571_s_at | −3.009919e−02 |
| X206637_at | 7.423640e−03 |
| X206666_at | 1.204182e−02 |
| X206687_s_at | −1.762981e−02 |
| X206715_at | 1.116688e−02 |
| X206804_at | 1.234660e−02 |
| X206898_at | −3.907050e−02 |
| X206978_at | −4.979548e−03 |
| X207076_s_at | 1.863378e−02 |
| X207238_s_at | −5.780355e−03 |
| X207277_at | 2.125419e−02 |
| X207458_at | 2.748393e−02 |
| X207540_s_at | −1.283397e−02 |
| X207574_s_at | −2.431727e−03 |
| X207651_at | 2.066787e−02 |
| X207655_s_at | 5.405012e−04 |
| X207677_s_at | 2.230816e−02 |
| X207843_x_at | 3.315503e−02 |
| X207861_at | 4.377097e−02 |
| X207977_s_at | 1.145723e−02 |
| X207992_s_at | 6.265461e−03 |
| X208296_x_at | −2.224775e−03 |
| X208306_x_at | −5.132684e−03 |
| X208335_s_at | 3.329359e−03 |
| X208450_at | 3.811124e−02 |
| X208747_s_at | −7.912263e−03 |
| X208885_at | −1.288364e−02 |
| X208894_at | 7.896947e−03 |
| X208981_at | −1.015968e−02 |
| X208983_s_at | −3.056594e−02 |
| X209083_at | 4.483265e−04 |
| X209138_x_at | 7.183442e−03 |
| X209193_at | 1.740572e−02 |
| X209195_s_at | −2.237887e−02 |
| X209202_s_at | 8.930806e−03 |
| X209312_x_at | −5.814581e−03 |
| X209480_at | 1.848455e−02 |
| X209542_x_at | 4.971766e−03 |
| X209603_at | 3.004576e−02 |
| X209606_at | 5.064024e−03 |
| X209612_s_at | 2.595736e−02 |
| X209613_s_at | 1.417618e−02 |
| X209670_at | −1.636542e−02 |
| X209671_x_at | 2.518135e−03 |
| X209685_s_at | −9.113030e−03 |
| X209687_at | −1.842487e−02 |
| X209710_at | 5.230642e−03 |
| X209734_at | −5.196811e−03 |
| X209774_x_at | 2.951233e−02 |
| X209795_at | −6.624457e−03 |
| X209813_x_at | 2.371992e−02 |
| X209899_s_at | 7.998200e−04 |
| X209924_at | 3.375216e−02 |
| X209949_at | 3.781916e−02 |
| X209970_x_at | 2.564418e−02 |
| X210038_at | 6.929319e−03 |
| X210072_at | 1.293525e−02 |
| X210251_s_at | 1.756383e−02 |
| X210260_s_at | −1.082042e−03 |
| X210319_x_at | −1.726655e−02 |
| X210375_at | 2.483769e−03 |
| X210554_s_at | −2.077789e−02 |
| X210835_s_at | −7.455480e−03 |
| X210915_x_at | 3.172003e−03 |
| X210972_x_at | 6.080266e−03 |
| X210982_s_at | 9.635875e−03 |
| X211066_x_at | −1.164103e−02 |
| X211144_x_at | 4.992137e−02 |
| X211200_s_at | −4.031722e−02 |
| X211339_s_at | −8.002311e−03 |
| X211366_x_at | 3.593000e−02 |
| X211367_s_at | 6.231728e−02 |
| X211368_s_at | 2.045696e−02 |
| X211654_x_at | 2.874123e−02 |
| X211742_s_at | −1.282275e−02 |
| X211796_s_at | 6.744624e−03 |
| X211902_x_at | 1.350420e−03 |
| X211990_at | −1.552756e−02 |
| X211991_s_at | −2.759634e−03 |
| X212067_s_at | −9.461685e−03 |
| X212233_at | 8.400376e−03 |
| X212538_at | 7.226797e−03 |
| X212587_s_at | −5.979921e−03 |
| X212588_at | −7.824565e−04 |
| X212592_at | 3.516261e−03 |
| X212657_s_at | 2.978857e−02 |
| X212671_s_at | −2.602076e−02 |
| X212713_at | 3.233683e−02 |
| X212763_at | −3.275778e−03 |
| X212886_at | 3.683799e−03 |
| X212977_at | 9.877644e−03 |
| X212998_x_at | 5.090511e−03 |
| X212999_x_at | 3.695828e−02 |
| X213007_at | −3.352605e−02 |
| X213008_at | −4.189798e−03 |
| X213068_at | 2.658290e−02 |
| X213095_x_at | 1.421934e−02 |
| X213193_x_at | 2.914348e−03 |
| X213475_s_at | −4.914118e−04 |
| X213537_at | −8.357035e−03 |
| X213539_at | 7.487953e−03 |
| X213560_at | −4.020759e−03 |
| X213566_at | 5.845149e−03 |
| X213603_s_at | −5.088264e−03 |
| X213618_at | 1.305106e−02 |
| X213652_at | 8.548287e−03 |
| X213819_s_at | 8.196495e−03 |
| X213831_at | 1.631173e−02 |
| X213888_s_at | 8.134177e−05 |
| X213975_s_at | −2.095655e−02 |
| X214023_x_at | −2.237865e−02 |
| X214038_at | 2.389479e−02 |
| X214181_x_at | −8.976012e−03 |
| X214450_at | 1.788553e−02 |
| X214470_at | 2.213217e−02 |
| X214617_at | 1.563590e−02 |
| X214669_x_at | 1.972879e−03 |
| X214677_x_at | 3.809399e−03 |
| X224719_at | 1.439531e−03 |
| X215051_x_at | 1.072135e−02 |
| X215121_x_at | 4.118073e−03 |
| X215193_x_at | 2.105556e−04 |
| X215223_s_at | 1.530709e−02 |
| X215379_x_at | 7.064224e−03 |
| X215561_s_at | −2.385977e−02 |
| X215806_x_at | 1.767858e−02 |
| X216155_at | −3.705145e−02 |
| X216191_s_at | 4.747897e−02 |
| X216194_s_at | −4.006836e−02 |
| X216714_at | 2.405266e−02 |
| X216841_s_at | 8.716417e−03 |
| X216920_s_at | 3.911396e−03 |
| X217028_at | 5.128469e−03 |
| X217138_x_at | 9.357220e−03 |
| X217143_s_at | 7.546655e−03 |
| X217147_s_at | −1.958024e−02 |
| X217478_s_at | −1.327997e−02 |
| X217525_at | −1.717356e−02 |
| X217629_at | 7.854937e−03 |
| X217767_at | −5.238491e−03 |
| X217995_at | −6.809023e−03 |
| X218002_s_at | 9.869321e−03 |
| X218035_s_at | 1.245016e−03 |
| X218145_at | −3.291834e−02 |
| X218170_at | −7.323517e−03 |
| X218322_s_at | −8.250237e−03 |
| X218499_at | 5.140903e−04 |
| X218736_s_at | 2.138520e−02 |
| X218739_at | 2.714745e−03 |
| X218764_at | −9.158772e−03 |
| X218802_at | 5.152377e−02 |
| X218805_at | −2.574003e−03 |
| X218854_at | 3.989147e−03 |
| X218899_s_at | −3.411423e−02 |
| X218950_at | −4.336176e−02 |
| X219054_at | 4.975575e−03 |
| X219093_at | 2.082412e−02 |
| X219213_at | −4.341432e−03 |
| X219243_at | 2.233332e−03 |
| X219368_at | −1.134073e−03 |
| X219440_at | 2.580567e−02 |
| X219454_at | 3.065280e−02 |
| X219505_at | 5.933108e−03 |
| X219519_s_at | 2.660637e−02 |
| X219525_at | 4.320312e−03 |
| X219528_s_at | 1.066797e−02 |
| X219631_at | −2.616546e−02 |
| X219666_at | 1.182519e−02 |
| X219681_s_at | 8.705075e−03 |
| X219710_at | −1.046131e−02 |
| X219737_s_at | −6.473357e−02 |
| X219777_at | −6.023843e−03 |
| X219789_at | 8.550214e−03 |
| X219926_at | −8.456673e−03 |
| X219938_s_at | 1.596546e−02 |
| X220005_at | 6.859902e−03 |
| X220066_at | 7.525339e−03 |
| X220330_s_at | 3.751801e−03 |
| X220485_s_at | 2.962480e−03 |
| X221081_s_at | −4.394394e−03 |
| X221087_s_at | 8.161216e−03 |
| X221477_s_at | 1.240372e−02 |
| X221651_x_at | 1.118009e−02 |
| X221671_x_at | 5.247343e−03 |
| X221698_s_at | −1.058842e−02 |
| X221756_at | −1.209965e−02 |
| X221760_at | −1.764419e−02 |
| X222108_at | 4.026916e−02 |
| X222142_at | −3.508958e−02 |
| X222484_s_at | −8.096999e−03 |
| X222496_s_at | −4.133924e−03 |
| X222592_s_at | 1.511240e−02 |
| X222725_s_at | 2.808005e−02 |
| X222780_s_at | −2.679162e−02 |
| X222838_at | 4.550716e−03 |
| X222895_s_at | −2.841100e−02 |
| X223044_at | 5.255602e−02 |
| X223058_at | 9.381819e−03 |
| X223059_s_at | 8.539393e−03 |
| X223168_at | −4.973917e−03 |
| X223235_s_at | 4.953002e−04 |
| X223280_x_at | 6.016285e−03 |
| X223322_at | −1.188607e−02 |
| X223361_at | −5.418802e−03 |
| X223395_at | 1.411881e−02 |
| X223484_at | 1.743558e−02 |
| X223809_at | 2.119400e−02 |
| X223827_at | −1.852331e−02 |
| X223922_x_at | 8.293530e−03 |
| X223924_at | −2.212120e−02 |
| X223952_x_at | 3.606709e−02 |
| X224356_x_at | 4.977366e−03 |
| X224358_s_at | −9.225797e−04 |
| X224451_x_at | −4.781080e−03 |
| X224516_s_at | −1.494258e−02 |
| X224710_at | −2.279989e−02 |
| X224771_at | −1.243246e−02 |
| X224772_at | −4.059490e−02 |
| X224773_at | −7.626004e−03 |
| X224774_s_at | −1.743111e−02 |
| X224795_x_at | 5.385788e−03 |
| X224859_at | −1.540871e−02 |
| X224896_s_at | −4.228341e−03 |
| X225502_at | 3.460051e−03 |
| X225802_at | −2.887415e−02 |
| X225895_at | 4.741893e−03 |
| X226043_at | 1.277772e−02 |
| X226068_at | 5.224876e−03 |
| X226117_at | 2.490399e−02 |
| X226218_at | −1.341873e−02 |
| X226219_at | 4.175131e−03 |
| X226303_at | 4.358878e−02 |
| X226382_at | 6.264514e−04 |
| X226459_at | 4.497883e−03 |
| X226625_at | 4.213244e−02 |
| X226659_at | −4.064464e−03 |
| X226697_at | −5.986636e−02 |
| X226818_at | 2.581249e−02 |
| X226841_at | 1.092228e−02 |
| X226865_at | −2.335927e−02 |
| X227035_x_at | 8.655280e−03 |
| X227231_at | −1.692593e−02 |
| X227253_at | 1.018350e−02 |
| X227265_at | 1.360205e−02 |
| X227346_at | −1.295157e−02 |
| X227361_at | −5.811243e−03 |
| X227458_at | 1.893847e−02 |
| X227584_at | −2.425646e−02 |
| X227609_at | 3.029790e−02 |
| X227640_s_at | −5.262288e−03 |
| X227780_s_at | 1.333635e−02 |
| X227791_at | 1.583168e−02 |
| X227983_at | −1.027572e−02 |
| X227995_at | −1.414606e−02 |
| X228054_at | −1.428632e−03 |
| X228071_at | −8.103128e−03 |
| X228094_at | 9.305227e−03 |
| X228153_at | 1.840560e−02 |
| X228339_at | 1.115556e−02 |
| X228362_s_at | 1.505306e−02 |
| X228372_at | −1.233323e−02 |
| X228376_at | 3.005129e−03 |
| X228427_at | 1.441232e−02 |
| X228532_at | −1.984789e−02 |
| X228552_s_at | 1.488213e−02 |
| X228563_at | 5.478160e−03 |
| X228660_x_at | −3.321971e−03 |
| X228776_at | 1.735059e−02 |
| X228812_at | 2.966052e−02 |
| X228858_at | 8.798795e−03 |
| X228869_at | 1.576960e−02 |
| X228908_s_at | 1.715903e−02 |
| X228964_at | 1.578864e−02 |
| X229127_at | 2.437139e−02 |
| X229163_at | −1.306446e−03 |
| X229367_s_at | 8.285081e−03 |
| X229390_at | 9.455414e−03 |
| X229391_s_at | 2.152998e−02 |
| X229543_at | 1.337527e−02 |
| X229625_at | 1.390034e−02 |
| X229723_at | 1.284379e−02 |
| X230233_at | 6.517225e−03 |
| X230391_at | −1.165179e−02 |
| X230538_at | −4.922326e−02 |
| X230728_at | −3.921071e−02 |
| X231032_at | 1.220460e−02 |
| X231262_at | 8.401904e−03 |
| X231577_s_at | −4.352510e−03 |
| X231882_at | −1.597680e−02 |
| X231929_at | −7.190517e−03 |
| X232001_at | −2.527809e−02 |
| X232024_at | 1.562502e−02 |
| X232234_at | 1.979601e−02 |
| X232311_at | −5.811767e−04 |
| X232313_at | 3.438576e−02 |
| X232476_at | −1.156007e−02 |
| X232543_x_at | −6.890048e−03 |
| X232617_at | 2.120826e−03 |
| X232746_at | −1.032038e−02 |
| X232843_s_at | −1.582714e−03 |
| X233123_at | 3.841746e−02 |
| X233562_at | −6.149035e−02 |
| X233955_x_at | −2.411061e−02 |
| X235175_at | −8.822903e−03 |
| X235238_at | −4.455537e−02 |
| X235306_at | −1.163963e−02 |
| X235391_at | 9.692727e−03 |
| X235421_at | −7.303012e−04 |
| X235639_at | −3.367098e−02 |
| X235688_s_at | −1.717805e−03 |
| X235804_at | −1.564079e−03 |
| X235831_at | 2.675921e−03 |
| X236203_at | −8.187551e−03 |
| X236280_at | 6.197497e−03 |
| X236295_s_at | 4.606321e−03 |
| X236583_at | 2.311931e−03 |
| X236908_at | −2.109408e−02 |
| X238439_at | 3.286334e−02 |
| X238488_at | 2.932061e−02 |
| X238544_at | 1.183049e−03 |
| X239196_at | 4.987304e−02 |
| X239237_at | 3.319884e−02 |
| X239744_at | −1.276710e−02 |
| X241671_x_at | −6.586756e−03 |
| X241701_at | −8.761918e−03 |
| X242458_at | −1.194306e−02 |
| X242546_at | 4.355118e−03 |
| X242874_at | 5.607680e−03 |
| X242881_x_at | −6.062508e−03 |
| X242986_at | 2.749620e−03 |
| X243099_at | 1.186699e−02 |
| X244023_at | −1.018460e−02 |
| X244061_at | 1.229564e−03 |
| X32128_at | 2.935196e−02 |
| X34210_at | 3.776762e−03 |
| X38149_at | 4.937711e−04 |
| X64064_at | −3.336735e−03 |

$lev
[1] “NR” “R”
$svd
[1] 7.995862
$N
[1] 29
$call
lda(formula = f ~ ., data = datatr)

APPENDIX C
R object of class naiveBayes for naïve Bayes clinical outcome
prediction of patients.
$apriori
Y

| NR | R |
|---|---|
| 17 | 12 |

| | | |
|---|---|---|
| Y | [,1] | [,2] |

tablestables$X1405_i_at
X1405_i_at

| | | |
|---|---|---|
| NR | −0.1639258 | 0.5851902 |
| R | 0.6748740 | 0.6631486 |

tables$X1552497_a_at
X1552497_a_at

| | | |
|---|---|---|
| NR | −0.3029996 | 0.6800071 |
| R | 0.6607734 | 0.8223674 |

tables$X1552612_at
X1552612_at

| | | |
|---|---|---|
| NR | −0.2523340 | 0.5485271 |
| R | 0.4450904 | 0.5434438 |

tables$X1552613_s_at
X1552613_s_at

| | | |
|---|---|---|
| NR | −0.2165343 | 0.5894093 |
| R | 0.4800677 | 0.4432806 |

tables$X1553102_a_at
X1553102_a_at

| | | |
|---|---|---|
| NR | −0.2474064 | 0.5834113 |
| R | 0.7071397 | 0.7121750 |

tables$X1553132_a_at
X1553132_a_at

| | | |
|---|---|---|
| NR | −0.1377405 | 0.3946696 |
| R | 0.4961300 | 0.4509575 |

tables$X1553313_s_at
X1553313_s_at

| | | |
|---|---|---|
| NR | 0.2393526 | 0.6100254 |
| R | −0.3838373 | 0.3940848 |

tables$X1553906_s_at
X1553906_s_at

| | | |
|---|---|---|
| NR | −0.3915645 | 0.6474232 |
| R | 0.2882052 | 0.3076821 |

tables$X1554240_a_at
X1554240_a_at

| | | |
|---|---|---|
| NR | 0.01780307 | 0.4865912 |
| R | 0.88377535 | 0.8029798 |

tables$X1554966_a_at
X1554966_a_at

| | | |
|---|---|---|
| NR | −0.1357018 | 0.3678133 |
| R | 0.6662706 | 0.6833596 |

tables$X1555229_a_at
X1555229_a_at

| | | |
|---|---|---|
| NR | −0.2263265 | 0.3882668 |
| R | 0.5467680 | 0.4361597 |

tables$X1555630_a_at
X1555630_a_at

| | | |
|---|---|---|
| NR | 0.2005512 | 0.3578418 |
| R | −0.6088957 | 0.7683101 |

tables$X1555756_a_at
X1555756_a_at

| | | |
|---|---|---|
| NR | −0.2186897 | 0.5247142 |
| R | 0.4061700 | 0.5187056 |

tables$X1555759_a_at
X1555759_a_at

| | | |
|---|---|---|
| NR | 0.08037061 | 0.5249017 |
| R | 0.85078348 | 0.7360755 |

tables$X1555812_a_at
X1555812_a_at

| | | |
|---|---|---|
| NR | −0.4493812 | 0.8437685 |
| R | 0.4087372 | 0.6145611 |

tables$X1555852_at
X1555852_at

| | | |
|---|---|---|
| NR | −0.3431720 | 0.6409197 |
| R | 0.3060830 | 0.4160286 |

tables$X1556185_a_at
X1556185_a_at

| | | |
|---|---|---|
| NR | −0.3542416 | 0.4573215 |
| R | 0.3876607 | 0.8113095 |

tables$X1556579_s_at
X1556579_s_at

| | | |
|---|---|---|
| NR | −0.08046679 | 0.2880584 |
| R | 0.58371780 | 0.7445854 |

tables$X1557116_at
X1557116_at

| | | |
|---|---|---|
| NR | −0.4470538 | 0.6892889 |
| R | 0.3267151 | 0.4124078 |

tables$X1557222_at
X1557222_at

| | | |
|---|---|---|
| NR | −0.04818824 | 0.402835 |
| R | 0.74360000 | 0.863980 |

tables$X1558034_s_at
X1558034_s_at

| | | |
|---|---|---|
| NR | −0.2487822 | 0.4220782 |
| R | 0.4252406 | 0.4675114 |

tables$X1558290_a_at
X1558290_a_at

| | | |
|---|---|---|
| NR | 0.3179399 | 0.5555339 |
| R | −0.4890204 | 0.4786095 |

tables$X1558586_at
X1558586_at

| | | |
|---|---|---|
| NR | −0.2298781 | 0.4177707 |
| R | 0.3950435 | 0.5416909 |

tables$X1558972_s_at
X1558972_s_at

| | | |
|---|---|---|
| NR | −0.2797971 | 0.5418070 |
| R | 0.4745703 | 0.5335784 |

tables$X1559263_s_at
X1559263_s_at

| | | |
|---|---|---|
| NR | −0.3833393 | 0.5703645 |
| R | 0.3494365 | 0.4676936 |

tables$X1559425_at
X1559425_at

| | | |
|---|---|---|
| NR | −0.2287316 | 0.5560199 |
| R | 0.4282525 | 0.4558128 |

tables$X1559584_a_at
X1559584_a_at

| | | |
|---|---|---|
| NR | −0.4622881 | 0.5846900 |
| R | 0.3021721 | 0.4028788 |

tables$X1562031_at
X1562031_at

| | | |
|---|---|---|
| NR | −0.3551523 | 0.4862453 |
| R | 0.1736934 | 0.4872335 |

tables$X1563461_at
X1563461_at

| | | |
|---|---|---|
| NR | 0.3566529 | 0.5512415 |
| R | −0.2706966 | 0.6822506 |

tables$X1563473_at
X1563473_at

| | | |
|---|---|---|
| NR | −0.4074615 | 0.5896901 |
| R | 0.2254661 | 0.4096997 |

tables$X1565602_at
X1565602_at

| | | |
|---|---|---|
| NR | 0.4490349 | 0.6371381 |
| R | −0.1986003 | 0.5670284 |

tables$X1568736_s_at
X1568736_s_at

| | | |
|---|---|---|
| NR | 0.6111740 | 0.9655418 |
| R | −0.1669456 | 0.3464934 |

tables$X1568822_at
X1568822_at

| | | |
|---|---|---|
| NR | 0.4611639 | 0.6061123 |
| R | −0.1089934 | 0.3960131 |

tables$X1569942_at
X1569942_at

| | | |
|---|---|---|
| NR | 0.4441859 | 0.7007245 |
| R | −0.2886340 | 0.3295043 |

tables$X200612_s_at
X200612_s_at

| | | |
|---|---|---|
| NR | 0.2054340 | 0.4467337 |
| R | −0.4553481 | 0.5048914 |

tables$X200904_at
X200904_at

| | | |
|---|---|---|
| NR | −0.2660950 | 0.4738856 |
| R | 0.3014091 | 0.4032104 |

tables$X200905_x_at
X200905_x_at

| | | |
|---|---|---|
| NR | −0.1327891 | 0.5236164 |
| R | 0.4735567 | 0.3948498 |

tables$X200953_s_at
X200953_s_at

| | | |
|---|---|---|
| NR | −0.2814847 | 0.8261769 |
| R | 0.5757425 | 0.6362976 |

tables$X201010_s_at
X201010_s_at

| | | |
|---|---|---|
| NR | −0.3613038 | 0.7929993 |
| R | 0.4944130 | 0.2677128 |

tables$X201137_s_at
X201137_s_at

| | | |
|---|---|---|
| NR | −0.3646008 | 0.7012958 |
| R | 0.3957589 | 0.4949475 |

tables$X201220_x_at
X201220_x_at

| | | |
|---|---|---|
| NR | 0.08672472 | 0.5177988 |
| R | −0.48143253 | 0.5513205 |

tables$X201236_s_at
X201236_s_at

| | | |
|---|---|---|
| NR | −0.1198028 | 0.5613972 |
| R | 0.5331348 | 0.5322952 |

tables$X201425_at
X201425_at

| | | |
|---|---|---|
| NR | −0.5116596 | 0.7193710 |
| R | 0.4964073 | 0.5304284 |

tables$X201474_s_at
X201474_s_at

| | | |
|---|---|---|
| NR | 0.6028068 | 0.6299064 |
| R | −0.2457276 | 0.3007111 |

tables$X201487_at
X201487_at

| | | |
|---|---|---|
| NR | −0.4225210 | 0.8252371 |
| R | 0.3171429 | 0.4591150 |

tables$X201497_x_at
X201497_x_at

| | | |
|---|---|---|
| NR | −0.3200855 | 0.4908815 |
| R | 2.5010094 | 2.6712499 |

tables$X201502_s_at
X201502_s_at

| | | |
|---|---|---|
| NR | −0.3308824 | 0.4722939 |
| R | 0.5454167 | 0.3159327 |

tables$X201531_at
X201531_at

| | | |
|---|---|---|
| NR | −0.1938551 | 0.3744374 |
| R | 0.5269050 | 0.5074891 |

tables$X201566_x_at
X201566_x_at

| | | |
|---|---|---|
| NR | −0.2981010 | 0.6780610 |
| R | 0.3509438 | 0.5014504 |

tables$X201720_s_at
X201720_s_at

| | | |
|---|---|---|
| NR | −0.1977420 | 0.6818082 |
| R | 0.5749931 | 0.7157990 |

tables$X201721_s_at
X201721_s_at

| | | |
|---|---|---|
| NR | −0.1677046 | 0.5133205 |
| R | 0.4231327 | 0.5084300 |

tables$X201804_x_at
X201804_x_at

| | | |
|---|---|---|
| NR | 0.1992091 | 0.4851767 |
| R | −0.4765795 | 0.5915133 |

tables$X201859_at
X201859_at

| | | |
|---|---|---|
| NR | −0.4216528 | 0.7283696 |
| R | 0.2486708 | 0.3163434 |

tables$X201939_at
X201939_at

| | | |
|---|---|---|
| NR | 0.2953508 | 0.5226393 |
| R | −0.3592031 | 0.5614863 |

tables$X202207_at
X202207_at

| | | |
|---|---|---|
| NR | −0.4088858 | 0.6063797 |
| R | 0.2159392 | 0.2923256 |

tables$X202255_s_at
X202255_s_at

| | | |
|---|---|---|
| NR | −0.5135289 | 0.8447912 |
| R | 0.3161547 | 0.6241535 |

tables$X202269_x_at
X202269_x_at

| | | |
|---|---|---|
| NR | −0.4070967 | 0.6699657 |
| R | 0.3070278 | 0.3134680 |

tables$X202270_at
X202270_at

| | | |
|---|---|---|
| NR | −0.3598381 | 0.7402801 |
| R | 0.4664866 | 0.4311290 |

tables$X202368_s_at
X202368_s_at

| | | |
|---|---|---|
| NR | 0.4828523 | 0.6622996 |
| R | −0.3498387 | 0.7337426 |

tables$X202369_s_at
X202369_s_at

| | | |
|---|---|---|
| NR | 0.3894707 | 0.7051767 |
| R | −0.4762401 | 0.5968765 |

tables$X202391_at
X202391_at

| | | |
|---|---|---|
| NR | −0.5342313 | 1.0124766 |
| R | 0.5933123 | 0.6491928 |

tables$X202510_s_at
X202510_s_at

| | | |
|---|---|---|
| NR | −0.6015966 | 0.9255712 |
| R | 0.4919973 | 0.6463912 |

tables$X202625_at
X202625_at

| | | |
|---|---|---|
| NR | −0.2142036 | 0.7804199 |
| R | 0.6620458 | 0.7148131 |

tables$X202643_s_at
X202643_s_at

| | | |
|---|---|---|
| NR | −0.4832904 | 0.5361050 |
| R | 0.3792320 | 0.4385239 |

tables$X202644_s_at
X202644_s_at

| | | |
|---|---|---|
| NR | −0.3513719 | 0.5742499 |
| R | 0.4307245 | 0.3838438 |

tables$X202687_s_at
X202687_s_at

| | | |
|---|---|---|
| NR | −0.4279648 | 0.7540136 |
| R | 0.3504315 | 0.4045005 |

tables$X202688_at
X202688_at

| | | |
|---|---|---|
| NR | −0.4163941 | 0.8163225 |
| R | 0.4027076 | 0.4386812 |

tables$X202948_at
X202948_at

| | | |
|---|---|---|
| NR | −0.5154460 | 0.7699102 |
| R | 0.4056092 | 0.4764355 |

tables$X202957_at
X202957_at

| | | |
|---|---|---|
| NR | −0.3735948 | 0.5616205 |
| R | 0.4472934 | 0.5131898 |

tables$X203413_at
X203413_at

| | | |
|---|---|---|
| NR | −0.3383329 | 0.5941084 |
| R | 0.3196087 | 0.6292591 |

tables$X203416_at
X203416_at

| | | |
|---|---|---|
| NR | −0.3141917 | 0.6552882 |
| R | 0.4391612 | 0.4251811 |

tables$X203471_s_at
X203471_s_at

| | | |
|---|---|---|
| NR | −0.2041767 | 0.5987281 |
| R | 0.6153344 | 0.5620431 |

tables$X203508_at
X203508_at

| | | |
|---|---|---|
| NR | −0.2695595 | 0.5179795 |
| R | 0.2812243 | 0.4853358 |

tables$X203523_at
X203523_at

| | | |
|---|---|---|
| NR | −0.3791594 | 0.8958684 |
| R | 0.8117565 | 0.9246945 |

tables$X203547_at
X203547_at

| | | |
|---|---|---|
| NR | −0.008562598 | 0.4462166 |
| R | 0.663436693 | 0.5733365 |

tables$X203665_at
X203665_at

| | | |
|---|---|---|
| NR | −0.2522801 | 0.5533137 |
| R | 0.6428083 | 0.7340506 |

tables$X203741_s_at
X203741_s_at

| | | |
|---|---|---|
| NR | −0.1850309 | 0.5721364 |
| R | 0.6581712 | 0.6185694 |

tables$X203761_at
X203761_at

| | | |
|---|---|---|
| NR | −0.1852189 | 0.5956581 |
| R | 0.5255720 | 0.4369002 |

tables$X203812_at
X203812_at

| | | |
|---|---|---|
| NR | 0.0587172 | 0.3136796 |
| R | 1.0676683 | 0.8555980 |

tables$X203868_s_at
X203868_s_at

| | | |
|---|---|---|
| NR | −0.3688355 | 0.6726738 |
| R | 0.2602381 | 0.3965858 |

tables$X203915_at
X203915_at

| | | |
|---|---|---|
| NR | −0.4061025 | 0.5236050 |
| R | 0.2301446 | 0.2415020 |

tables$X203932_at
X203932_at

| | | |
|---|---|---|
| NR | −0.2494244 | 0.6622825 |
| R | 0.4463418 | 0.3757829 |

tables$X204057_at
X204057_at

| | | |
|---|---|---|
| NR | −0.3274287 | 0.5906957 |
| R | 0.4847696 | 0.4283121 |

tables$X204070_at
X204070_at

| | | |
|---|---|---|
| NR | −0.2719985 | 0.6097243 |
| R | 0.4394442 | 0.3954333 |

tables$X204116_at
X204116_at

| | | |
|---|---|---|
| NR | −0.2653059 | 0.4657288 |
| R | 0.5320249 | 0.3827573 |

tables$X204118_at
X204118_at

| | | |
|---|---|---|
| NR | −0.2188632 | 0.5465435 |
| R | 0.4397206 | 0.4841528 |

tables$X204135_at
X204135_at

| | | |
|---|---|---|
| NR | −0.2511983 | 0.3620796 |
| R | 0.5582305 | 0.6587871 |

tables$X204204_at
X204204_at

| | | |
|---|---|---|
| NR | −0.2476304 | 0.5677727 |
| R | 0.5469376 | 0.5015591 |

tables$X204220_at
X204220_at

| | | |
|---|---|---|
| NR | −0.4512334 | 0.9464671 |
| R | 0.4470878 | 0.4911261 |

tables$X204222_s_at
X204222_s_at

| | | |
|---|---|---|
| NR | −0.2858410 | 0.8009875 |
| R | 0.4771543 | 0.4557632 |

tables$X204224_s_at
X204224_s_at

| | | |
|---|---|---|
| NR | −0.4034291 | 0.533261 |
| R | 0.4591664 | 0.324186 |

tables$X204233_s_at
X204233_s_at

| | | |
|---|---|---|
| NR | 0.1725203 | 0.526541 |
| R | −0.4473613 | 0.498801 |

tables$X204236_at
X204236_at

| | | |
|---|---|---|
| NR | −0.3405471 | 0.7438102 |
| R | 0.4577420 | 0.4830432 |

tables$X204249_s_at
X204249_s_at

| | | |
|---|---|---|
| NR | −0.2439368 | 0.6736956 |
| R | 0.5798690 | 0.5938803 |

tables$X204411_at
X204411_at

| | | |
|---|---|---|
| NR | −0.1561360 | 0.5046013 |
| R | 0.5348875 | 0.6639757 |

tables$X204438_at
X204438_at

| | | |
|---|---|---|
| NR | −0.2155729 | 0.8013524 |
| R | 0.5238707 | 0.5465740 |

tables$X204502_at
X204502_at

| | | |
|---|---|---|
| NR | −0.3163975 | 0.5660414 |
| R | 0.3122723 | 0.3372872 |

tables$X204512_at
X204512_at

| | | |
|---|---|---|
| NR | −0.3823994 | 0.7656201 |
| R | 0.3405082 | 0.3302137 |

tables$X204533_at
X204533_at

| | | |
|---|---|---|
| NR | −0.3432882 | 0.5411550 |
| R | 0.2820025 | 0.3529044 |

tables$X204613_at
X204613_at

| | | |
|---|---|---|
| NR | −0.2921141 | 0.5365989 |
| R | 0.3433918 | 0.4634024 |

tables$X204628_s_at
X204628_s_at

| | | |
|---|---|---|
| NR | 0.2412563 | 0.5057318 |
| R | −0.4238494 | 0.4487938 |

tables$X204642_at
X204642_at

| | | |
|---|---|---|
| NR | −0.3125616 | 0.7411993 |
| R | 0.6952398 | 1.0691429 |

tables$X204655_at
X204655_at

| | | |
|---|---|---|
| NR | −0.2716510 | 0.6208783 |
| R | 0.5579874 | 0.5183030 |

tables$X204661_at
X204661_at

| | | |
|---|---|---|
| NR | −0.1558546 | 0.4835795 |
| R | 0.5868508 | 0.3065469 |

tables$X204670_x_at
X204670_x_at

| | | |
|---|---|---|
| NR | −0.3190668 | 0.6529019 |
| R | 0.4161121 | 0.3104711 |

tables$X204687_at
X204687_at

| | | |
|---|---|---|
| NR | 0.1167838 | 0.3453266 |
| R | 1.0001048 | 0.8753104 |

tables$X204724_s_at
X204724_s_at

| | | |
|---|---|---|
| NR | 0.5223107 | 0.6822646 |
| R | −0.1768374 | 0.3008967 |

tables$X204774_at
X204774_at

| | | |
|---|---|---|
| NR | −0.3545849 | 0.6395887 |
| R | 0.1938959 | 0.3271488 |

tables$X204778_x_at
X204778_x_at

| | | |
|---|---|---|
| NR | 0.3141006 | 0.5671928 |
| R | −0.4937885 | 0.7083350 |

tables$X204834_at
X204834_at

| | | |
|---|---|---|
| NR | −0.2829585 | 0.5026786 |
| R | 0.2222514 | 0.2582449 |

tables$X204846_at
X204846_at

| | | |
|---|---|---|
| NR | 0.1129389 | 0.4384144 |
| R | 0.7672991 | 0.7249123 |

tables$X204894_s_at
X204894_s_at

| | | |
|---|---|---|
| NR | −0.04169668 | 0.5285664 |
| R | 0.90340136 | 1.0066307 |

tables$X204897_at
X204897_at

| | | |
|---|---|---|
| NR | −0.5753379 | 0.7174762 |
| R | 0.2097906 | 0.3253104 |

tables$X204912_at
X204912_at

| | | |
|---|---|---|
| NR | −0.1581854 | 0.4616605 |
| R | 0.4406020 | 0.3673491 |

tables$X204923_at
X204923_at

| | | |
|---|---|---|
| NR | −0.1810564 | 0.5020173 |
| R | 0.4762799 | 0.5956602 |

tables$X205027_s_at
X205027_s_at

| | | |
|---|---|---|
| NR | −0.2115740 | 0.8053652 |
| R | 0.9541627 | 0.9478824 |

tables$X205039_s_at
X205039_s_at

| | | |
|---|---|---|
| NR | −0.07447284 | 0.6105666 |
| R | 0.75225499 | 0.7306959 |

tables$X205081_at
X205081_at

| | | |
|---|---|---|
| NR | −0.4320239 | 0.6844631 |
| R | 0.4515831 | 0.6544651 |

tables$X205159_at
X205159_at

| | | |
|---|---|---|
| NR | −0.3955032 | 0.7064307 |
| R | 0.4559497 | 0.4821926 |

tables$X205225_at
X205225_at

| | | |
|---|---|---|
| NR | −0.4166425 | 0.5776734 |
| R | 0.4113626 | 0.4147703 |

tables$X205226_at
X205226_at

| | | |
|---|---|---|
| NR | −0.07833695 | 0.4854712 |
| R | 0.44132335 | 0.7107620 |

tables$X205251_at
X205251_at

| | | |
|---|---|---|
| NR | −0.3110220 | 0.6899898 |
| R | 0.5272399 | 0.6253671 |

tables$X205285_s_at
X205285_s_at

| | | |
|---|---|---|
| NR | −0.04432586 | 0.5000114 |
| R | 0.80925537 | 0.9021956 |

tables$X205392_s_at
X205392_s_at

| | | |
|---|---|---|
| NR | −0.4220336 | 0.4956664 |
| R | 0.4657780 | 0.3693635 |

tables$X205403_at
X205403_at

| | | |
|---|---|---|
| NR | −0.2005647 | 0.4806835 |
| R | 0.5966667 | 0.7091052 |

tables$X205419_at
X205419_at

| | | |
|---|---|---|
| NR | −0.2813820 | 0.5751165 |
| R | 0.5056242 | 0.4918400 |

tables$X205421_at
X205421_at

| | | |
|---|---|---|
| NR | 0.07840435 | 0.3868954 |
| R | 0.86698828 | 0.8643222 |

tables$X205440_s_at
X205440_s_at

| | | |
|---|---|---|
| NR | −0.3108277 | 0.5223075 |
| R | 0.3749281 | 0.5730182 |

tables$X205484_at
X205484_at

| | | |
|---|---|---|
| NR | −0.2739962 | 0.5607660 |
| R | 0.4613413 | 0.4462779 |

tables$X205488_at
X205488_at

| | | |
|---|---|---|
| NR | −0.211302 | 0.5156343 |
| R | 0.451222 | 0.3725271 |

tables$X205559_s_at
X205559_s_at

| | | |
|---|---|---|
| NR | −0.1967785 | 0.4797350 |
| R | 0.4925489 | 0.5104237 |

tables$X205569_at
X205569_at

| | | |
|---|---|---|
| NR | −0.3112147 | 0.5708862 |
| R | 0.3356277 | 0.4533386 |

tables$X205624_at
X205624_at

| | | |
|---|---|---|
| NR | 0.005427652 | 0.4382683 |
| R | 0.763112333 | 0.7396355 |

tables$X205685_at
X205685_at

| | | |
|---|---|---|
| NR | −0.1946623 | 0.5288449 |
| R | 0.4467601 | 0.4223863 |

tables$X205696_s_at
X205696_s_at

| | | |
|---|---|---|
| NR | −0.01091462 | 0.3565887 |
| R | 0.77207850 | 0.7974577 |

tables$X205758_at
X205758_at

| | | |
|---|---|---|
| NR | −0.2386930 | 0.4171697 |
| R | 0.4248193 | 0.3603297 |

tables$X205786_s_at
X205786_s_at

| | | |
|---|---|---|
| NR | −0.4968271 | 0.8934640 |
| R | 0.4149078 | 0.5488123 |

tables$X205831_at
X205831_at

| | | |
|---|---|---|
| NR | 0.04447927 | 0.4259325 |
| R | 0.69838627 | 0.4833483 |

tables$X205841_at
X205841_at

| | | |
|---|---|---|
| NR | −0.5900546 | 0.7313311 |
| R | 0.3136276 | 0.5142651 |

tables$X205844_at
X205844_at

| | | |
|---|---|---|
| NR | −0.1423775 | 0.4883837 |
| R | 0.4235432 | 0.4438472 |

tables$X205890_s_at
X205890_s_at

| | | |
|---|---|---|
| NR | −0.3511421 | 0.4822404 |
| R | 0.3120630 | 0.2244957 |

tables$X205987_at
X205987_at

| | | |
|---|---|---|
| NR | −0.09413415 | 0.3978789 |
| R | 0.59504052 | 0.5335613 |

tables$X206082_at
X206082_at

| | | |
|---|---|---|
| NR | −0.4540809 | 0.6785699 |
| R | 0.4716106 | 0.5496119 |

tables$X206099_at
X206099_at

| | | |
|---|---|---|
| NR | −0.0531401 | 0.3438675 |
| R | 0.5295345 | 0.5201337 |

tables$X206118_at
X206118_at

| | | |
|---|---|---|
| NR | −0.2013207 | 0.4883106 |
| R | 0.5011802 | 0.4695632 |

tables$X206134_at
X206134_at

| | | |
|---|---|---|
| NR | −0.2474317 | 0.6355258 |
| R | 0.3651382 | 0.4198818 |

tables$X206170_at
X206170_at

| | | |
|---|---|---|
| NR | −0.4274713 | 0.5078651 |
| R | 0.3104491 | 0.3419569 |

tables$X206204_at
X206204_at

| | | |
|---|---|---|
| NR | −0.02057820 | 0.2205198 |
| R | 0.94769151 | 0.9338282 |

tables$X206295_at
X206295_at

| | | |
|---|---|---|
| NR | −0.4222879 | 0.5539909 |
| R | 0.2869025 | 0.3697476 |

tables$X206385_s_at
X206385_s_at

| | | |
|---|---|---|
| NR | −0.1448051 | 0.4343064 |
| R | 0.5421084 | 0.6951307 |

tables$X206407_s_at
X206407_s_at

| | | |
|---|---|---|
| NR | −0.0965811 | 0.4714271 |
| R | 0.5253937 | 0.6785424 |

tables$X206545_at
X206545_at

| | | |
|---|---|---|
| NR | −0.2348002 | 0.6434845 |
| R | 0.3898586 | 0.5979256 |

tables$X206571_s_at
X206571_s_at

| | | |
|---|---|---|
| NR | 0.2964052 | 0.5266454 |
| R | −0.5021825 | 0.7307312 |

tables$X206637_at
X206637_at

| | | |
|---|---|---|
| NR | −0.4929224 | 0.8293232 |
| R | 0.5561408 | 0.7415308 |

tables$X206666_at
X206666_at

| | | |
|---|---|---|
| NR | −0.3815276 | 0.4518748 |
| R | 0.3020703 | 0.1922297 |

tables$X206687_s_at
X206687_s_at

| | | |
|---|---|---|
| NR | −0.3592461 | 0.6018756 |
| R | 0.3975221 | 0.5022615 |

tables$X206715_at
X206715_at

| | | |
|---|---|---|
| NR | −0.2541760 | 0.6982257 |
| R | 0.4082547 | 0.3259496 |

tables$X206804_at
X206804_at

| | | |
|---|---|---|
| NR | 0.1482064 | 0.5068781 |
| R | 0.9166047 | 0.7147839 |

tables$X206898_at
X206898_at

| | | |
|---|---|---|
| NR | 0.1056709 | 0.4682096 |
| R | −0.5435822 | 0.5520821 |

tables$X206978_at
X206978_at

| | | |
|---|---|---|
| NR | −0.3496069 | 0.5339659 |
| R | 0.2843234 | 0.3287630 |

tables$X207076_s_at
X207076_s_at

| | | |
|---|---|---|
| NR | −0.02361779 | 0.5694192 |
| R | 1.31170006 | 1.6322450 |

tables$X207238_s_at
X207238_s_at

| | | |
|---|---|---|
| NR | −0.2571791 | 0.6182402 |
| R | 0.5089045 | 0.4856678 |

tables$X207277_at
X207277_at

| | | |
|---|---|---|
| NR | −0.3525872 | 0.7509863 |
| R | 0.4181711 | 0.4255746 |

tables$X207458_at
X207458_at

| | | |
|---|---|---|
| NR | 0.2419331 | 0.5735746 |
| R | −0.3710235 | 0.4350871 |

tables$X207540_s_at
X207540_s_at

| | | |
|---|---|---|
| NR | −0.2692932 | 0.5488522 |
| R | 0.3711915 | 0.4966252 |

tables$X207574_s_at
X207574_s_at

| | | |
|---|---|---|
| NR | −0.3958191 | 0.7713632 |
| R | 0.4298086 | 0.3017609 |

tables$X207651_at
X207651_at

| | | |
|---|---|---|
| NR | −0.3101854 | 0.4462963 |
| R | 0.5275557 | 0.2883934 |

tables$X207655_s_at
X207655_s_at

| | | |
|---|---|---|
| NR | −0.2656028 | 0.6249668 |
| R | 0.4038824 | 0.4984622 |

tables$X207677_s_at
X207677_s_at

| | | |
|---|---|---|
| NR | −0.1067724 | 0.6536107 |
| R | 0.6251422 | 0.4735116 |

tables$X207843_x_at
X207843_x_at

| | | |
|---|---|---|
| NR | −0.1980640 | 0.4840094 |
| R | 0.6923162 | 1.1190386 |

tables$X207861_at
X207861_at

| | | |
|---|---|---|
| NR | −0.1260775 | 0.3508021 |
| R | 0.6778354 | 0.7911460 |

tables$X207977_s_at
X207977_s_at

| | | |
|---|---|---|
| NR | −0.02630196 | 0.3452417 |
| R | 0.72601762 | 0.5798353 |

tables$X207992_s_at
X207992_s_at

| | | |
|---|---|---|
| NR | −0.2962782 | 0.6213732 |
| R | 0.4065298 | 0.3562586 |

tables$X208296_x_at
X208296_x_at

| | | |
|---|---|---|
| NR | −0.3202962 | 0.6726712 |
| R | 0.4715979 | 0.3630417 |

tables$X208306_x_at
X208306_x_at

| | | |
|---|---|---|
| NR | −0.3404445 | 0.6697022 |
| R | 0.3560108 | 0.2684112 |

tables$X208335_s_at
X208335_s_at

| | | |
|---|---|---|
| NR | −0.6832978 | 0.8622729 |
| R | 0.3887120 | 0.6307942 |

tables$X208450_at
X208450_at

| | | |
|---|---|---|
| NR | 0.05646932 | 0.6161496 |
| R | 0.97349919 | 0.7211533 |

tables$X208747_s_at
X208747_s_at

| | | |
|---|---|---|
| NR | −0.7269106 | 1.215719 |
| R | 0.6146325 | 0.602124 |

tables$X208885_at
X208885_at

| | | |
|---|---|---|
| NR | −0.4878552 | 0.6970401 |
| R | 0.3000394 | 0.4132369 |

tables$X208894_at
X208894_at

| | | |
|---|---|---|
| NR | −0.2782715 | 0.5325159 |
| R | 0.3660963 | 0.3734728 |

tables$X208981_at
X208981_at

| | | |
|---|---|---|
| NR | −0.3974853 | 0.7581136 |
| R | 0.3901547 | 0.3829096 |

tables$X208983_s_at
X208983_s_at

| | | |
|---|---|---|
| NR | −0.3682743 | 0.6238660 |
| R | 0.3346874 | 0.3924348 |

tables$X209083_at
X209083_at

| | | |
|---|---|---|
| NR | 0.1258308 | 0.6751235 |
| R | 0.9235844 | 0.8069453 |

tables$X209138_x_at
X209138_x_at

| | | |
|---|---|---|
| NR | −0.1402324 | 0.4308172 |
| R | 0.3394879 | 0.3946554 |

tables$X209193_at
X209193_at

| | | |
|---|---|---|
| NR | −0.2565556 | 0.5468921 |
| R | 0.4873494 | 0.4649883 |

tables$X209195_s_at
X209195_s_at

| | | |
|---|---|---|
| NR | 0.1800756 | 0.4677919 |
| R | −0.4026223 | 0.3673557 |

tables$X209202_s_at
X209202_s_at

| | | |
|---|---|---|
| NR | 0.1964649 | 0.7379767 |
| R | −0.5653081 | 0.5741860 |

tables$X209312_x_at
X209312_x_at

| | | |
|---|---|---|
| NR | −0.2928795 | 0.6652902 |
| R | 0.4275265 | 0.3568548 |

tables$X209480_at
X209480_at

| | | |
|---|---|---|
| NR | −0.2841051 | 0.4380402 |
| R | 0.2693738 | 0.4024957 |

tables$X209542_x_at
X209542_x_at

| | | |
|---|---|---|
| NR | −0.2363103 | 0.6011656 |
| R | 0.4349808 | 0.5810748 |

tables$X209603_at
X209603_at

| | | |
|---|---|---|
| NR | −0.4656699 | 0.4521237 |
| R | 0.3621330 | 0.6118526 |

tables$X209606_at
X209606_at

| | | |
|---|---|---|
| NR | −0.2611256 | 0.5441526 |
| R | 0.5627781 | 0.3290949 |

tables$X209612_s_at
X209612_s_at

| | | |
|---|---|---|
| NR | −0.2482860 | 0.4645433 |
| R | 0.3891742 | 0.5803480 |

tables$X209613_s_at
X209613_s_at

| | | |
|---|---|---|
| NR | −0.3246706 | 0.4195264 |
| R | 0.2137530 | 0.4363348 |

tables$X209670_at
X209670_at

| | | |
|---|---|---|
| NR | −0.3904387 | 0.5757881 |
| R | 0.4960604 | 0.4286288 |

tables$X209671_x_at
X209671_x_at

| | | |
|---|---|---|
| NR | 0.005489902 | 0.4958616 |
| R | 0.946682909 | 0.6669064 |

tables$X209685_s_at
X209685_s_at

| | | |
|---|---|---|
| NR | −0.3380362 | 0.6105057 |
| R | 0.3675781 | 0.4181149 |

tables$X209687_at
X209687_at

| | | |
|---|---|---|
| NR | −0.5180091 | 0.5954837 |
| R | 0.3297076 | 0.5807315 |

tables$X209710_at
X209710_at

| | | |
|---|---|---|
| NR | −0.2868007 | 0.3941463 |
| R | 0.4039716 | 0.7769142 |

tables$X209734_at
X209734_at

| | | |
|---|---|---|
| NR | −0.2771824 | 0.5584141 |
| R | 0.3480342 | 0.4269202 |

tables$X209774_x_at
X209774_x_at

| | | |
|---|---|---|
| NR | −0.5154483 | 0.5084051 |
| R | 0.3327482 | 0.2052511 |

tables$X209795_at
X209795_at

| | | |
|---|---|---|
| NR | −0.5014384 | 0.5485146 |
| R | 0.2126023 | 0.2563472 |

tables$X209813_x_at
X209813_x_at

| | | |
|---|---|---|
| NR | −0.02446552 | 0.4178348 |
| R | 0.66828816 | 0.5665816 |

tables$X209899_s_at
X209899_s_at

| | | |
|---|---|---|
| NR | 0.2607015 | 0.6973904 |
| R | −0.4270344 | 0.5920830 |

tables$X209924_at
X209924_at

| | | |
|---|---|---|
| NR | −0.1412661 | 0.5234151 |
| R | 0.3997205 | 0.5359769 |

tables$X209949_at
X209949_at

| | | |
|---|---|---|
| NR | −0.2882600 | 0.4631077 |
| R | 0.4047160 | 0.3303257 |

tables$X209970_x_at
X209970_x_at

| | | |
|---|---|---|
| NR | −0.4439942 | 0.6955252 |
| R | 0.1102440 | 0.2548926 |

tables$X210038_at
X210038_at

| | | |
|---|---|---|
| NR | 0.1317110 | 0.4768708 |
| R | 0.8494560 | 0.7295487 |

tables$X210072_at
X210072_at

| | | |
|---|---|---|
| NR | 0.01156063 | 0.3737049 |
| R | 0.57481931 | 0.4627490 |

tables$X210251_s_at
X210251_s_at

| | | |
|---|---|---|
| NR | 0.2240660 | 0.4879510 |
| R | −0.6597691 | 0.6547051 |

tables$X210260_s_at
X210260_s_at

| | | |
|---|---|---|
| NR | −0.3640910 | 0.6337100 |
| R | 0.4487179 | 0.3720418 |

tables$X210319_x_at
X210319_x_at

| | | |
|---|---|---|
| NR | −0.08072798 | 0.5440481 |
| R | 0.83296501 | 1.2085067 |

tables$X210375_at
X210375_at

| | | |
|---|---|---|
| NR | −0.0859139 | 0.2736614 |
| R | 0.9924649 | 1.0291749 |

tables$X210554_s_at
X210554_s_at

| | | |
|---|---|---|
| NR | 0.1522882 | 0.5489999 |
| R | −0.5083098 | 0.6130962 |

tables$X210835_s_at
X210835_s_at

| | | |
|---|---|---|
| NR | 0.2332468 | 0.5454729 |
| R | −0.4438314 | 0.6253821 |

tables$X210915_x_at
X210915_x_at

| | | |
|---|---|---|
| NR | −0.2269420 | 0.466748 |
| R | 0.5087007 | 0.360080 |

tables$X210972_x_at
X210972_x_at

| | | |
|---|---|---|
| NR | −0.2866590 | 0.4272028 |
| R | 0.5598691 | 0.4527992 |

tables$X210982_s_at
X210982_s_at

| | | |
|---|---|---|
| NR | −0.3512586 | 0.5571462 |
| R | 0.3419340 | 0.3732178 |

tables$X211066_x_at
X211066_x_at

| | | |
|---|---|---|
| NR | 0.2422814 | 0.6284310 |
| R | −0.5086901 | 0.7771297 |

tables$X211144_x_at
X211144_x_at

| | | |
|---|---|---|
| NR | 0.05087736 | 0.3119915 |
| R | 0.70598903 | 0.4822038 |

tables$X211200_s_at
X211200_s_at

| | | |
|---|---|---|
| NR | 0.2580184 | 0.5530421 |
| R | −0.4922700 | 0.5506593 |

tables$X211339_s_at
X211339_s_at

| | | |
|---|---|---|
| NR | −0.2296132 | 0.5247178 |
| R | 0.5767409 | 0.5988969 |

tables$X211366_x_at
X211366_x_at

| | | |
|---|---|---|
| NR | −0.3681938 | 0.6327750 |
| R | 0.1432735 | 0.2631491 |

tables$X211367_s_at
X211367_s_at

| | | |
|---|---|---|
| NR | −0.1751026 | 0.5723239 |
| R | 0.3320629 | 0.2562800 |

tables$X211368_s_at
X211368_s_at

| | | |
|---|---|---|
| NR | −0.51886132 | 0.7515452 |
| R | 0.07358956 | 0.2643141 |

tables$X211654_x_at
X211654_x_at

| | | |
|---|---|---|
| NR | −0.3850022 | 0.5573621 |
| R | 0.3719847 | 0.3143733 |

tables$X211742_s_at
X211742_s_at

| | | |
|---|---|---|
| NR | −0.2540869 | 0.6521022 |
| R | 0.4880575 | 0.5506316 |

tables$X211796_s_at
X211796_s_at

| | | |
|---|---|---|
| NR | −0.3093201 | 0.4536705 |
| R | 0.4624019 | 0.3008702 |

tables$X211902_x_at
X211902_x_at

| | | |
|---|---|---|
| NR | −0.2792212 | 0.5535743 |
| R | 0.6836665 | 0.6034058 |

tables$X211990_at
X211990_at

| | | |
|---|---|---|
| NR | −0.4590253 | 0.8582217 |
| R | 0.4251869 | 0.4814630 |

tables$X211991_s_at
X211991_s_at

| | | |
|---|---|---|
| NR | −0.2092869 | 0.6666829 |
| R | 0.5537245 | 0.3967729 |

tables$X212067_s_at
X212067_s_at

| | | |
|---|---|---|
| NR | −0.4803278 | 0.7242299 |
| R | 0.3558230 | 0.6889846 |

tables$X212233_at
X212233_at

| | | |
|---|---|---|
| NR | 0.1442229 | 0.5131552 |
| R | −0.4879059 | 0.7214358 |

tables$X212538_at
X212538_at

| | | |
|---|---|---|
| NR | −0.3849016 | 0.7522011 |
| R | 0.6516464 | 0.6995619 |

tables$X212587_s_at
X212587_s_at

| | | |
|---|---|---|
| NR | −0.3531934 | 0.5890246 |
| R | 0.3718118 | 0.3431055 |

tables$X212588_at
X212588_at

| | | |
|---|---|---|
| NR | −0.5662804 | 0.8162279 |
| R | 0.4008046 | 0.4444447 |

tables$X212592_at
X212592_at

| | | |
|---|---|---|
| NR | −0.3409693 | 0.4849486 |
| R | 0.2387676 | 0.2127384 |

tables$X212657_s_at
X212657_s_at

| | | |
|---|---|---|
| NR | −0.3419394 | 0.6083771 |
| R | 0.2570024 | 0.4217927 |

tables$X212671_s_at
X212671_s_at

| | | |
|---|---|---|
| NR | −0.5140866 | 0.8193929 |
| R | 0.5074176 | 0.3776582 |

tables$X212713_at
X212713_at

| | | |
|---|---|---|
| NR | 0.03767197 | 0.3268940 |
| R | 0.74731430 | 0.8347694 |

tables$X212763_at
X212763_at

| | | |
|---|---|---|
| NR | 0.2888164 | 0.8845972 |
| R | −0.6529463 | 0.9707572 |

tables$X212886_at
X212886_at

| | | |
|---|---|---|
| NR | −0.3765371 | 0.7892555 |
| R | 0.6201350 | 0.6611937 |

tables$X212977_at
X212977_at

| | | |
|---|---|---|
| NR | −0.4688782 | 0.8767720 |
| R | 0.5293928 | 0.6187406 |

tables$X212998_x_at
X212998_x_at

| | | |
|---|---|---|
| NR | −0.2035514 | 0.6457962 |
| R | 0.5296660 | 0.3118830 |

tables$X212999_x_at
X212999_x_at

| | | |
|---|---|---|
| NR | −0.2290011 | 0.4910183 |
| R | 0.4331640 | 0.3718106 |

tables$X213007_at
X213007_at

| | | |
|---|---|---|
| NR | 0.2632353 | 0.6401111 |
| R | −0.3996142 | 0.4037179 |

tables$X213008_at
X213008_at

| | | |
|---|---|---|
| NR | 0.4761995 | 0.7015473 |
| R | −0.4947303 | 0.8235263 |

tables$X213068_at
X213068_at

| | | |
|---|---|---|
| NR | −0.1758656 | 0.4044001 |
| R | 0.4193472 | 0.3911686 |

tables$X213095_x_at
X213095_x_at

| | | |
|---|---|---|
| NR | −0.3502315 | 0.8054069 |
| R | 0.3616843 | 0.3443190 |

tables$X213193_x_at
X213193_x_at

| | | |
|---|---|---|
| NR | −0.2370856 | 0.4636341 |
| R | 0.5058580 | 0.3505763 |

tables$X213475_s_at
X213475_s_at

| | | |
|---|---|---|
| NR | −0.2137215 | 0.4617051 |
| R | 0.4040370 | 0.4908631 |

tables$X213537_at
X213537_at

| | | |
|---|---|---|
| NR | −0.3674984 | 0.5888660 |
| R | 0.3219144 | 0.5336377 |

tables$X213539_at
X213539_at

| | | |
|---|---|---|
| NR | −0.3813796 | 0.5040301 |
| R | 0.3415072 | 0.2577433 |

tables$X213560_at
X213560_at

| | | |
|---|---|---|
| NR | −0.5055000 | 0.7230271 |
| R | 0.2766383 | 0.2303920 |

tables$X213566_at
X213566_at

| | | |
|---|---|---|
| NR | −0.2311022 | 0.5691307 |
| R | 0.3719339 | 0.4376776 |

tables$X213603_s_at
X213603_s_at

| | | |
|---|---|---|
| NR | −0.3061774 | 0.6126919 |
| R | 0.3508772 | 0.5262723 |

tables$X213618_at
X213618_at

| | | |
|---|---|---|
| NR | −0.3695683 | 0.681454 |
| R | 0.4212985 | 0.554157 |

tables$X213652_at
X213652_at

| | | |
|---|---|---|
| NR | −0.2586966 | 0.4605599 |
| R | 0.3270410 | 0.8177604 |

tables$X213819_s_at
X213819_s_at

| | | |
|---|---|---|
| NR | 0.00122895 | 0.3972336 |
| R | −0.64986825 | 0.9085849 |

tables$X213831_at
X213831_at

| | | |
|---|---|---|
| NR | −0.3533186 | 0.4215363 |
| R | 0.1563120 | 0.3886529 |

tables$X213888_s_at
X213888_s_at

| | | |
|---|---|---|
| NR | −0.2086168 | 0.5349552 |
| R | 0.4303498 | 0.4269851 |

tables$X213975_s_at
X213975_s_at

| | | |
|---|---|---|
| NR | −0.4990363 | 0.6803409 |
| R | 0.1878550 | 0.2710180 |

tables$X214023_x_at
X214023_x_at

| | | |
|---|---|---|
| NR | 0.1848739 | 0.7174998 |
| R | −0.4325008 | 0.4810479 |

tables$X214038_at
X214038_at

| | | |
|---|---|---|
| NR | −0.3255701 | 0.6706608 |
| R | 0.3599967 | 0.6048520 |

tables$X214181_x_at
X214181_x_at

| | | |
|---|---|---|
| NR | −0.3415893 | 0.6522400 |
| R | 0.2853699 | 0.2763122 |

tables$X214450_at
X214450_at

| | | |
|---|---|---|
| NR | −0.1562932 | 0.2807857 |
| R | 0.8434281 | 0.8613678 |

tables$X214470_at
X214470_at

| | | |
|---|---|---|
| NR | −0.2839688 | 0.4885156 |
| R | 0.4212807 | 0.3750679 |

tables$X214617_at
X214617_at

| | | |
|---|---|---|
| NR | −0.3487562 | 0.4518071 |
| R | 0.2125207 | 0.4242197 |

tables$X214669_x_at
X214669_x_at

| | | |
|---|---|---|
| NR | −0.1915274 | 0.4981106 |
| R | 0.3359622 | 0.4156267 |

tables$X214677_x_at
X214677_x_at

| | | |
|---|---|---|
| NR | −0.2087490 | 0.4787112 |
| R | 0.3128333 | 0.3867513 |

tables$X214719_at
X214719_at

| | | |
|---|---|---|
| NR | −0.4511937 | 0.6070570 |
| R | 0.2711287 | 0.3505487 |

tables$X215051_x_at
X215051_x_at

| | | |
|---|---|---|
| NR | −0.3597073 | 0.8514825 |
| R | 0.4054302 | 0.3092948 |

tables$X215121_x_at
X215121_x_at

| | | |
|---|---|---|
| NR | −0.1634545 | 0.4642640 |
| R | 0.3410072 | 0.4014670 |

tables$X215193_x_at
X215193_x_at

| | | |
|---|---|---|
| NR | −0.2667421 | 0.7134086 |
| R | 0.4777644 | 0.4030902 |

tables$X215223_s_at
X215223_s_at

| | | |
|---|---|---|
| NR | −0.3883071 | 0.4910665 |
| R | 0.2565276 | 0.4364856 |

tables$X215379_x_at
X215379_x_at

| | | |
|---|---|---|
| NR | −0.09709331 | 0.4452099 |
| R | 0.39542823 | 0.4092484 |

tables$X215561_s_at
X215561_s_at

| | | |
|---|---|---|
| NR | −0.2711632 | 0.4077480 |
| R | 0.7072055 | 0.9320206 |

tables$X215806_x_at
X215806_x_at

| | | |
|---|---|---|
| NR | −0.007773052 | 0.3665788 |
| R | 0.661454102 | 0.4785205 |

tables$X216155_at
X216155_at

| | | |
|---|---|---|
| NR | 0.5119447 | 0.6151164 |
| R | −0.1726881 | 0.3347342 |

tables$X216191_s_at
X216191_s_at

| | | |
|---|---|---|
| NR | −0.01475148 | 0.4433394 |
| R | 0.67109688 | 0.5079869 |

tables$X216194_s_at
X216194_s_at

| | | |
|---|---|---|
| NR | 0.1641682 | 0.3935104 |
| R | −0.3667475 | 0.4613841 |

tables$X216714_at
X216714_at

| | | |
|---|---|---|
| NR | −0.1657215 | 0.6600958 |
| R | 0.5669961 | 0.9931878 |

tables$X216841_s_at
X216841_s_at

| | | |
|---|---|---|
| NR | −0.2378813 | 0.7063683 |
| R | 0.5838156 | 0.5499012 |

tables$X216920_s_at
X216920_s_at

| | | |
|---|---|---|
| NR | −0.3626922 | 0.5129860 |
| R | 0.4424685 | 0.4026252 |

tables$X217028_at
X217028_at

| | | |
|---|---|---|
| NR | −0.3936253 | 0.7242766 |
| R | 0.2813510 | 0.3842826 |

tables$X217138_x_at
X217138_x_at

| | | |
|---|---|---|
| NR | 0.06341764 | 0.4690319 |
| R | 0.61978484 | 0.5049407 |

tables$X217143_s_at
X217143_s_at

| | | |
|---|---|---|
| NR | −0.1709594 | 0.6329777 |
| R | 0.6395395 | 0.5675738 |

tables$X217147_s_at
X217147_s_at

| | | |
|---|---|---|
| NR | −0.1919006 | 0.3907915 |
| R | 0.4594408 | 0.3635919 |

tables$X217478_s_at
X217478_s_at

| | | |
|---|---|---|
| NR | −0.3453566 | 0.7688595 |
| R | 0.5037971 | 0.4475461 |

tables$X217525_at
X217525_at

| | | |
|---|---|---|
| NR | −0.2639411 | 0.8866455 |
| R | 0.6075298 | 0.5960824 |

tables$X217629_at
X217629_at

| | | |
|---|---|---|
| NR | −0.1348654 | 0.671148 |
| R | 0.8011176 | 1.134701 |

tables$X217767_at
X217767_at

| | | |
|---|---|---|
| NR | −0.4316702 | 0.6383017 |
| R | 0.3296425 | 0.3962954 |

tables$X217995_at
X217995_at

| | | |
|---|---|---|
| NR | −0.6500190 | 1.0622844 |
| R | 0.3656452 | 0.3596701 |

tables$X218002_s_at
X218002_s_at

| | | |
|---|---|---|
| NR | −0.2371548 | 0.4549764 |
| R | 0.3590465 | 0.5928193 |

tables$X218035_s_at
X218035_s_at

| | | |
|---|---|---|
| NR | −0.3792486 | 0.6876336 |
| R | 0.3685478 | 0.4296116 |

tables$X218145_at
X218145_at

| | | |
|---|---|---|
| NR | 0.5407030 | 0.7961511 |
| R | −0.5330237 | 0.7914334 |

tables$X218170_at
X218170_at

| | | |
|---|---|---|
| NR | −0.2778944 | 0.5053423 |
| R | 0.5047588 | 0.6555875 |

tables$X218322_s_at
X218322_s_at

| | | |
|---|---|---|
| NR | −0.4368137 | 0.5136086 |
| R | 0.3249243 | 0.2575257 |

tables$X218459_at
X218499_at

| | | |
|---|---|---|
| NR | −0.2574311 | 0.6676495 |
| R | 0.4022312 | 0.4066238 |

tables$X218736_s_at
X218736_s_at

| | | |
|---|---|---|
| NR | −0.03615144 | 0.4561428 |
| R | 1.08677338 | 1.1597104 |

tables$X218739_at
X218739_at

| | | |
|---|---|---|
| NR | −0.02247695 | 0.4787563 |
| R | 0.98329866 | 1.2407971 |

tables$X218764_at
X218764_at

| | | |
|---|---|---|
| NR | −0.4099779 | 0.4830029 |
| R | 0.3567869 | 0.4038910 |

tables$X218802_at
X218802_at

| | | |
|---|---|---|
| NR | −0.3430126 | 0.4453773 |
| R | 0.5970457 | 0.3951568 |

tables$X218805_at
X218805_at

| | | |
|---|---|---|
| NR | −0.3671355 | 0.5708859 |
| R | 0.4517210 | 0.2930704 |

tables$X218854_at
X218854_at

| | | |
|---|---|---|
| NR | −0.3745559 | 0.7096822 |
| R | 0.4739352 | 0.5603528 |

tables$X218899_s_at
X218899_s_at

| | | |
|---|---|---|
| NR | 0.3671446 | 0.5909345 |
| R | −0.5393210 | 0.4381099 |

tables$X218950_at
X218950_at

| | | |
|---|---|---|
| NR | 0.2691863 | 0.4573251 |
| R | −0.6111111 | 0.6474936 |

tables$X219054_at
X219054_at

| | | |
|---|---|---|
| NR | 0.01472793 | 0.584855 |
| R | 2.49050475 | 2.683467 |

tables$X219093_at
X219093_at

| | | |
|---|---|---|
| NR | −0.2971580 | 0.4688604 |
| R | 0.5384298 | 0.6366817 |

tables$X219213_at
X219213_at

| | | |
|---|---|---|
| NR | −0.1731958 | 0.4047427 |
| R | 0.6423430 | 0.7730134 |

tables$X219243_at
X219243_at

| | | |
|---|---|---|
| NR | −0.5195758 | 0.7578959 |
| R | 0.4000750 | 0.3801539 |

tables$X219368_at
X219368_at

| | | |
|---|---|---|
| NR | −0.1679820 | 0.3075502 |
| R | 0.6324772 | 0.6637538 |

tables$X219440_at
X219440_at

| | | |
|---|---|---|
| NR | 0.08196161 | 0.2856868 |
| R | 0.87878388 | 0.7545222 |

tables$X219454_at
X219454_at

| | | |
|---|---|---|
| NR | −0.1553019 | 0.3324367 |
| R | 0.6028235 | 0.7768835 |

tables$X219505_at
X219505_at

| | | |
|---|---|---|
| NR | −0.335471 | 0.5884701 |
| R | 0.331203 | 0.4762600 |

tables$X219519_s_at
X219519_s_at

| | | |
|---|---|---|
| NR | −0.3029785 | 0.6574268 |
| R | 0.4245899 | 0.6733826 |

tables$X219525_at
X219525_at

| | | |
|---|---|---|
| NR | −0.4849566 | 0.6164310 |
| R | 0.2295082 | 0.3984913 |

tables$X219528_s_at
X219528_s_at

| | | |
|---|---|---|
| NR | −0.2597881 | 0.4319096 |
| R | 0.4928448 | 0.5142826 |

tables$X219631_at
X219631_at

| | | |
|---|---|---|
| NR | 0.5705786 | 0.6831084 |
| R | −0.2784402 | 0.3830630 |

tables$X219666_at
X219666_at

| | | |
|---|---|---|
| NR | −0.5430705 | 0.7930909 |
| R | 0.2839697 | 0.3631652 |

tables$X219681_s_at
X219681_s_at

| | | |
|---|---|---|
| NR | −0.417274 | 0.7733850 |
| R | 0.376309 | 0.5736019 |

tables$X219710_at
X219710_at

| | | |
|---|---|---|
| NR | 0.2636804 | 0.4736957 |
| R | −0.3241903 | 0.5279440 |

tables$X219737_s_at
X219737_s_at

| | | |
|---|---|---|
| NR | 0.3596773 | 0.5146777 |
| R | −0.2409581 | 0.4405703 |

tables$X219777_at
X219777_at

| | | |
|---|---|---|
| NR | −0.3965677 | 0.5974954 |
| R | 0.5026195 | 0.4178151 |

tables$X219789_at
X219789_at

| | | |
|---|---|---|
| NR | 0.0625228 | 0.3929864 |
| R | 1.5326986 | 1.6918348 |

tables$X219926_at
X219926_at

| | | |
|---|---|---|
| NR | 0.2428123 | 0.5989463 |
| R | −0.6869766 | 0.9798973 |

tables$X219938_s_at
X219938_s_at

| | | |
|---|---|---|
| NR | −0.4681105 | 0.5841168 |
| R | 0.3093149 | 0.2386032 |

tables$X220005_at
X220005_at

| | | |
|---|---|---|
| NR | −0.09036238 | 0.5172305 |
| R | 0.62083607 | 0.5403729 |

tables$X220066_at
X220066_at

| | | |
|---|---|---|
| NR | −0.2813542 | 0.4958868 |
| R | 0.2894245 | 0.2549095 |

tables$X220330_s_at
X220330_s_at

| | | |
|---|---|---|
| NR | −0.1838030 | 0.5516329 |
| R | 0.4155977 | 0.3566931 |

tables$X220485_s_at
X220485_s_at

| | | |
|---|---|---|
| NR | −0.2516505 | 0.989380 |
| R | 1.2092181 | 1.358517 |

tables$X221081_s_at
X221081_s_at

| | | |
|---|---|---|
| NR | −0.3406222 | 0.5260212 |
| R | 0.2204497 | 0.3080250 |

tables$X221087_s_at
X221087_s_at

| | | |
|---|---|---|
| NR | −0.3965314 | 0.6050086 |
| R | 0.2956204 | 0.3544964 |

tables$X221477_s_at
X221477_s_at

| | | |
|---|---|---|
| NR | −0.3833003 | 0.7680959 |
| R | 0.4128233 | 0.5675033 |

tables$X221651_x_at
X221651_x_at

| | | |
|---|---|---|
| NR | −0.3022085 | 0.4778491 |
| R | 0.2004842 | 0.2922928 |

tables$X221671_x_at
X221671_x_at

| | | |
|---|---|---|
| NR | −0.3000931 | 0.4932356 |
| R | 0.2268629 | 0.2801547 |

tables$X221698_s_at
X221698_s_at

| | | |
|---|---|---|
| NR | −0.6376038 | 0.9130990 |
| R | 0.3423824 | 0.3837021 |

tables$X221756_at
X221756_at

| | | |
|---|---|---|
| NR | −0.1597059 | 0.4810294 |
| R | 0.7144792 | 0.8100807 |

tables$X221760_at
X221760_at

| | | |
|---|---|---|
| NR | −0.3895238 | 0.6667009 |
| R | 0.2589523 | 0.4120362 |

tables$X222108_at
X222108_at

| | | |
|---|---|---|
| NR | −0.3578270 | 0.4644883 |
| R | 0.3207759 | 0.3326270 |

tables$X222142_at
X222142_at

| | | |
|---|---|---|
| NR | −0.1876780 | 0.4933581 |
| R | 0.3824561 | 0.4368395 |

tables$X222484_s_at
X222484_s_at

| | | |
|---|---|---|
| NR | −0.4729730 | 0.6937840 |
| R | 0.3761529 | 0.5801888 |

tables$X222496_s_at
X222496_s_at

| | | |
|---|---|---|
| NR | −0.4144878 | 0.6266733 |
| R | 0.2909259 | 0.2895369 |

tables$X222592_s_at
X222592_s_at

| | | |
|---|---|---|
| NR | −0.2780972 | 0.5068942 |
| R | 0.4106129 | 0.2951602 |

tables$X222725_s_at
X222725_s_at

| | | |
|---|---|---|
| NR | −0.004398634 | 0.5694045 |
| R | 1.150669643 | 1.2645302 |

tables$X222780_s_at
X222780_s_at

| | | |
|---|---|---|
| NR | 0.4139560 | 0.5398394 |
| R | −0.4224571 | 0.2983037 |

tables$X222838_at
X222838_at

| | | |
|---|---|---|
| NR | −0.2978748 | 0.5457765 |
| R | 0.2590990 | 0.2508078 |

tables$X222895_s_at
X222895_s_at

| | | |
|---|---|---|
| NR | −0.4561043 | 0.5248071 |
| R | 0.1985346 | 0.3539992 |

tables$X223044_at
X223044_at

| | | |
|---|---|---|
| NR | −0.4249287 | 0.7273378 |
| R | 0.3345815 | 0.5282742 |

tables$X223058_at
X223058_at

| | | |
|---|---|---|
| NR | −0.5976291 | 0.7565737 |
| R | 0.2836632 | 0.4557419 |

tables$X223059_s_at
X223059_s_at

| | | |
|---|---|---|
| NR | −0.2707661 | 0.6996713 |
| R | 0.5245500 | 0.4391394 |

tables$X223168_at
X223168_at

| | | |
|---|---|---|
| NR | −0.3429218 | 0.5205996 |
| R | 0.2543322 | 0.3823076 |

tables$X223235_s_at
X223235_s_at

| | | |
|---|---|---|
| NR | −0.2858518 | 0.4718234 |
| R | 0.6748075 | 0.7147878 |

tables$X223280_x_at
X223280_x_at

| | | |
|---|---|---|
| NR | −0.5988057 | 0.8558921 |
| R | 0.2763766 | 0.4726699 |

tables$X223322_at
X223322_at

| | | |
|---|---|---|
| NR | −0.5307109 | 0.7623257 |
| R | 0.4863893 | 0.5655322 |

tables$X223361_at
X223361_at

| | | |
|---|---|---|
| NR | −0.2076597 | 0.5924435 |
| R | 0.3858025 | 0.5723073 |

tables$X223395_at
X223395_at

| | | |
|---|---|---|
| NR | −0.2616053 | 0.5452143 |
| R | 0.4342364 | 0.5337485 |

tables$X223484_at
X223484_at

| | | |
|---|---|---|
| NR | −0.2440592 | 0.3975480 |
| R | 0.4224246 | 0.4741438 |

tables$X223809_at
X223809_at

| | | |
|---|---|---|
| NR | −0.2342077 | 0.5645847 |
| R | 0.3558237 | 0.3616038 |

tables$X223827_at
X223827_at

| | | |
|---|---|---|
| NR | 0.60366334 | 0.8225446 |
| R | −0.06966146 | 0.1454527 |

tables$X223922_x_at
X223922_x_at

| | | |
|---|---|---|
| NR | −0.4981749 | 0.7014087 |
| R | 0.2234232 | 0.3896602 |

tables$X223924_at
X223924_at

| | | |
|---|---|---|
| NR | 0.4849658 | 0.6301636 |
| R | −0.1459645 | 0.4704890 |

tables$X223952_x_at
X223952_x_at

| | | |
|---|---|---|
| NR | 0.1264371 | 0.4236437 |
| R | 0.8628858 | 0.9031651 |

tables$X224356_x_at
X224356_x_at

| | | |
|---|---|---|
| NR | −0.5122008 | 0.7621637 |
| R | 0.2604697 | 0.4291461 |

tables$X224358_s_at
X224358_s_at

| | | |
|---|---|---|
| NR | −0.4371837 | 0.8408400 |
| R | 0.3218548 | 0.5126749 |

tables$X224451_x_at
X224451_x_at

| | | |
|---|---|---|
| NR | −0.2604966 | 0.5285926 |
| R | 0.4760714 | 0.4279992 |

tables$X224516_s_at
X224516_s_at

| | | |
|---|---|---|
| NR | 0.2968854 | 0.6541436 |
| R | −0.6889220 | 0.9012319 |

tables$X224710_at
X224710_at

| | | |
|---|---|---|
| NR | 0.2230392 | 0.3643719 |
| R | −0.7978563 | 0.9064005 |

tables$X224771_at
X224771_at

| | | |
|---|---|---|
| NR | 0.4329412 | 0.8903073 |
| R | −0.4421469 | 0.6725526 |

tables$X224772_at
X224772_at

| | | |
|---|---|---|
| NR | 0.1916542 | 0.4865887 |
| R | −0.5224768 | 0.4540955 |

tables$X224773_at
X224773_at

| | | |
|---|---|---|
| NR | 0.3157972 | 0.5857999 |
| R | −0.4979135 | 0.4238849 |

tables$X224774_s_at
X224774_s_at

| | | |
|---|---|---|
| NR | 0.5703417 | 0.6486541 |
| R | −0.5030708 | 0.5503160 |

tables$X224795_x_at
X224795_x_at

| | | |
|---|---|---|
| NR | −0.3054091 | 0.4872416 |
| R | 0.2135411 | 0.2854565 |

tables$X224859_at
X224859_at

| | | |
|---|---|---|
| NR | 0.4830222 | 0.7137397 |
| R | −0.3679703 | 0.6853105 |

tables$X224896_s_at
X224896_s_at

| | | |
|---|---|---|
| NR | 0.1816808 | 0.7977231 |
| R | −0.6408326 | 0.5850013 |

tables$X225502_at
X225502_at

| | | |
|---|---|---|
| NR | −0.3910820 | 0.6073778 |
| R | 0.4852237 | 0.4201402 |

tables$X225802_at
X225802_at

| | | |
|---|---|---|
| NR | 0.1344681 | 0.5702449 |
| R | −0.4950925 | 0.5294767 |

tables$X225895_at
X225895_at

| | | |
|---|---|---|
| NR | −0.3277170 | 0.7566241 |
| R | 0.6412089 | 0.7479682 |

tables$X226043_at
X226043_at

| | | |
|---|---|---|
| NR | 0.1973572 | 0.6004289 |
| R | −0.3655970 | 0.4868833 |

tables$X226068_at
X226068_at

| | | |
|---|---|---|
| NR | −0.3490564 | 0.6019387 |
| R | 0.4335378 | 0.4792696 |

tables$X226117_at
X226117_at

| | | |
|---|---|---|
| NR | −0.2837194 | 0.5233255 |
| R | 0.4253069 | 0.3317653 |

tables$X226218_at
X226218_at

| | | |
|---|---|---|
| NR | −0.3859418 | 0.6090051 |
| R | 0.4404194 | 0.3778062 |

tables$X226219_at
X226219_at

| | | |
|---|---|---|
| NR | −0.2943873 | 0.5216637 |
| R | 0.4004941 | 0.4131372 |

tables$X226303_at
X226303_at

| | | |
|---|---|---|
| NR | −0.04068867 | 0.4037450 |
| R | 0.73077236 | 0.8465357 |

tables$X226382_at
X226382_at

| | | |
|---|---|---|
| NR | −0.3657410 | 0.4652645 |
| R | 0.2469253 | 0.4366802 |

tables$X226459_at
X226459_at

| | | |
|---|---|---|
| NR | −0.3680501 | 0.7660787 |
| R | 0.5040254 | 0.5894990 |

tables$X226625_at
X226625_at

| | | |
|---|---|---|
| NR | −0.3757333 | 0.7664846 |
| R | 0.5166961 | 0.4684207 |

tables$X226659_at
X226659_at

| | | |
|---|---|---|
| NR | −0.4911180 | 0.6832209 |
| R | 0.2612643 | 0.5381065 |

tables$X226697_at
X226697_at

| | | |
|---|---|---|
| NR | 0.1958473 | 0.3711059 |
| R | −0.5652399 | 0.4049380 |

tables$X226818_at
X226818_at

| | | |
|---|---|---|
| NR | −0.2318967 | 0.6119576 |
| R | 0.4987669 | 0.3954684 |

tables$X226841_at
X226841_at

| | | |
|---|---|---|
| NR | −0.2979441 | 0.7024396 |
| R | 0.5392790 | 0.4413953 |

tables$X226865_at
X226865_at

| | | |
|---|---|---|
| NR | −0.1559129 | 0.5471301 |
| R | 0.5041605 | 0.5099762 |

tables$X227035_x_at
X227035_x_at

| | | |
|---|---|---|
| NR | 0.2005227 | 0.4921465 |
| R | −0.3809524 | 0.4152160 |

tables$X227231_at
X227231_at

| | | |
|---|---|---|
| NR | 0.2353572 | 0.7279505 |
| R | −0.6509830 | 0.8181286 |

tables$X227253_at
X227253_at

| | | |
|---|---|---|
| NR | 0.03671536 | 0.4958005 |
| R | 1.03309945 | 1.0290932 |

tables$X227265_at
X227265_at

| | | |
|---|---|---|
| NR | −0.3512747 | 0.6026026 |
| R | 0.3283856 | 0.3401296 |

tables$X227346_at
X227346_at

| | | |
|---|---|---|
| NR | −0.3365397 | 0.7439603 |
| R | 0.6189716 | 0.5315020 |

tables$X227361_at
X227361_at

| | | |
|---|---|---|
| NR | −0.2481734 | 0.4900125 |
| R | 0.2745731 | 0.3724106 |

tables$X227458_at
X227458_at

| | | |
|---|---|---|
| NR | −0.1210425 | 0.5832677 |
| R | 0.5061539 | 0.5297024 |

tables$X227584_at
X227584_at

| | | |
|---|---|---|
| NR | 0.1614340 | 0.6512560 |
| R | −0.6792229 | 0.5867806 |

tables$X227609_at
X227609_at

| | | |
|---|---|---|
| NR | −0.2162779 | 0.6215554 |
| R | 0.4224651 | 0.4449701 |

tables$X227640_s_at
X227640_s_at

| | | |
|---|---|---|
| NR | 0.2476183 | 0.5414889 |
| R | −0.3764071 | 0.4938285 |

tables$X227780_s_at
X227780_s_at

| | | |
|---|---|---|
| NR | −0.3434434 | 0.6237700 |
| R | 0.4192693 | 0.6791114 |

tables$X227791_at
X227791_at

| | | |
|---|---|---|
| NR | −0.2588648 | 0.5252067 |
| R | 0.4328271 | 0.4393930 |

tables$X227983_at
X227983_at

| | | |
|---|---|---|
| NR | −0.3232334 | 0.6182386 |
| R | 0.3661930 | 0.5316841 |

tables$X227995_at
X227995_at

| | | |
|---|---|---|
| NR | −0.1696409 | 0.3982272 |
| R | 0.4873422 | 0.5629170 |

tables$X228054_at
X228054_at

| | | |
|---|---|---|
| NR | 0.1996287 | 0.5784055 |
| R | −0.4191671 | 0.4113732 |

tables$X228071_at
X228071_at

| | | |
|---|---|---|
| NR | −0.5040346 | 0.7357555 |
| R | 0.4529897 | 0.4488694 |

tables$X228094_at
X228094_at

| | | |
|---|---|---|
| NR | −0.5037012 | 0.7256858 |
| R | 0.5085461 | 0.4165603 |

tables$X228153_at
X228153_at

| | | |
|---|---|---|
| NR | −0.3116782 | 0.6938566 |
| R | 0.3443284 | 0.3237526 |

tables$X228339_at
X228339_at

| | | |
|---|---|---|
| NR | −0.2039593 | 0.7344429 |
| R | 0.5985043 | 0.7672987 |

tables$X228362_s_at
X228362_s_at

| | | |
|---|---|---|
| NR | −0.3778104 | 0.5378346 |
| R | 0.2342191 | 0.2730511 |

tables$X228372_at
X228372_at

| | | |
|---|---|---|
| NR | −0.4350057 | 0.7819102 |
| R | 0.4561920 | 0.3911495 |

tables$X228376_at
X228376_at

| | | |
|---|---|---|
| NR | −0.3453361 | 0.7260925 |
| R | 0.5292299 | 0.5379290 |

tables$X228427_at
X228427_at

| | | |
|---|---|---|
| NR | 0.1593717 | 0.7055373 |
| R | −0.6106282 | 0.8316641 |

tables$X228532_at
X228532_at

| | | |
|---|---|---|
| NR | −0.3794859 | 0.6481241 |
| R | 0.5000793 | 0.4684207 |

tables$X228552_s_at
X228552_s_at

| | | |
|---|---|---|
| NR | 0.1167790 | 0.4798513 |
| R | −0.5450925 | 0.7268180 |

tables$X228563_at
X228563_at

| | | |
|---|---|---|
| NR | 0.4236616 | 0.5619516 |
| R | −0.3316479 | 0.5347978 |

tables$X228660_x_at
X228660_x_at

| | | |
|---|---|---|
| NR | 0.5804684 | 0.7378901 |
| R | −0.1713584 | 0.4483440 |

tables$X228776_at
X228776_at

| | | |
|---|---|---|
| NR | 0.06752481 | 0.5251283 |
| R | −0.80628566 | 0.8423271 |

tables$X228812_at
X228812_at

| | | |
|---|---|---|
| NR | −0.3127907 | 0.6313677 |
| R | 0.5074128 | 0.6516242 |

tables$X228858_at
X228858_at

| | | |
|---|---|---|
| NR | −0.2190644 | 0.7355751 |
| R | 0.8241261 | 0.8754638 |

tables$X228869_at
X228869_at

| | | |
|---|---|---|
| NR | −0.3255735 | 0.5223796 |
| R | 0.3751149 | 0.4806706 |

tables$X228908_s_at
X228908_s_at

| | | |
|---|---|---|
| NR | 0.3383439 | 0.4459491 |
| R | −0.2935832 | 0.7121557 |

tables$X228964_at
X228964_at

| | | |
|---|---|---|
| NR | −0.3612173 | 0.5362295 |
| R | 0.2810782 | 0.4326503 |

tables$X229127_at
X229127_at

| | | |
|---|---|---|
| NR | −0.4082796 | 0.5831644 |
| R | 0.3339318 | 0.8204140 |

tables$X229163_at
X229163_at

| | | |
|---|---|---|
| NR | 0.4273831 | 0.7589842 |
| R | −0.2717283 | 0.5013583 |

tables$X229367_s_at
X229367_s_at

| | | |
|---|---|---|
| NR | −0.05987742 | 0.3928434 |
| R | 0.56349206 | 0.5126817 |

tables$X229390_at
X229390_at

| | | |
|---|---|---|
| NR | −0.4894938 | 0.6210411 |
| R | 0.1935497 | 0.2963602 |

tables$X229391_s_at
X229391_s_at

| | | |
|---|---|---|
| NR | −0.4084651 | 0.5373347 |
| R | 0.2172787 | 0.2684421 |

tables$X229543_at
X229543_at

| | | |
|---|---|---|
| NR | −0.3788834 | 0.5874714 |
| R | 0.2662277 | 0.2904183 |

tables$X229625_at
X229625_at

| | | |
|---|---|---|
| NR | −0.3005955 | 0.6601697 |
| R | 0.5530654 | 0.4811529 |

tables$X229723_at
X229723_at

| | | |
|---|---|---|
| NR | −0.2920168 | 0.5053402 |
| R | 0.4696342 | 0.4191172 |

tables$X230233_at
X230233_at

| | | |
|---|---|---|
| NR | −0.4143786 | 0.5555824 |
| R | 0.3424073 | 0.3923358 |

tables$X230391_at
X230391_at

| | | |
|---|---|---|
| NR | −0.3501254 | 0.8265678 |
| R | 0.4900276 | 0.4781532 |

tables$X230538_at
X230538_at

| | | |
|---|---|---|
| NR | 0.1276822 | 0.3932100 |
| R | −0.5735748 | 0.7224135 |

tables$X230728_at
X230728_at

| | | |
|---|---|---|
| NR | 0.3244249 | 0.4668566 |
| R | −0.2743950 | 0.5139070 |

tables$X231032_at
X231032_at

| | | |
|---|---|---|
| NR | 0.43019858 | 0.5207535 |
| R | −0.04588641 | 0.3560523 |

tables$X231262_at
X231262_at

| | | |
|---|---|---|
| NR | 0.05461486 | 0.2495836 |
| R | 1.32806434 | 1.1081493 |

tables$X231577_s_at
X231577_s_at

| | | |
|---|---|---|
| NR | −0.3279081 | 0.7462895 |
| R | 0.4819051 | 0.4270797 |

tables$X231882_at
X231882_at

| | | |
|---|---|---|
| NR | 0.1390358 | 0.4492620 |
| R | −0.4280975 | 0.4211427 |

tables$X231929_at
X231929_at

| | | |
|---|---|---|
| NR | −0.4149811 | 0.6028339 |
| R | 0.3275499 | 0.4633949 |

tables$X232001_at
X232001_at

| | | |
|---|---|---|
| NR | −0.4082551 | 0.5622408 |
| R | 0.2120889 | 0.4452512 |

tables$X232024_at
X232024_at

| | | |
|---|---|---|
| NR | −0.2767578 | 0.5975732 |
| R | 0.4329087 | 0.3644723 |

tables$X232234_at
X232234_at

| | | |
|---|---|---|
| NR | −0.2485230 | 0.5169943 |
| R | 0.5378690 | 0.4919297 |

tables$X232311_at
X232311_at

| | | |
|---|---|---|
| NR | −0.2207241 | 0.5017714 |
| R | 0.4996502 | 0.4849287 |

tables$X232313_at
X232313_at

| | | |
|---|---|---|
| NR | 0.09690295 | 0.2976671 |
| R | 0.96479280 | 0.7382041 |

tables$X232476_at
X232476_at

| | | |
|---|---|---|
| NR | 0.1978897 | 0.4874151 |
| R | −0.3000000 | 0.4272541 |

tables$X232543_x_at
X232543_x_at

| | | |
|---|---|---|
| NR | −0.1276883 | 0.6158742 |
| R | 0.6695534 | 0.5964269 |

tables$X232617_at
X232617_at

| | | |
|---|---|---|
| NR | −0.2190263 | 0.6769132 |
| R | 0.5988644 | 0.4031826 |

tables$X232746_at
X232746_at

| | | |
|---|---|---|
| NR | −0.3742172 | 0.5931449 |
| R | 0.3407643 | 0.5742058 |

tables$X232843_s_at
X232843_s_at

| | | |
|---|---|---|
| NR | −0.3175189 | 0.5332408 |
| R | 0.3950617 | 0.3666760 |

tables$X233123_at
X233123_at

| | | |
|---|---|---|
| NR | −0.2015742 | 0.5531279 |
| R | 0.4654343 | 0.7103440 |

tables$X233562_at
X233562_at

| | | |
|---|---|---|
| NR | 0.2792368 | 0.4159995 |
| R | −0.2877796 | 0.4229988 |

tables$X233955_x_at
X233955_x_at

| | | |
|---|---|---|
| NR | 0.3290657 | 0.6359343 |
| R | −0.3166667 | 0.5295163 |

tables$X235175_at
X235175_at

| | | |
|---|---|---|
| NR | −0.1311223 | 0.4904278 |
| R | 0.4533666 | 0.4676313 |

tables$X235238_at
X235238_at

| | | |
|---|---|---|
| NR | 0.1248063 | 0.3770484 |
| R | −0.5772758 | 0.7499223 |

tables$X235306_at
X235306_at

| | | |
|---|---|---|
| NR | −0.5666624 | 1.047734 |
| R | 0.4955309 | 0.534125 |

tables$X235391_at
X235391_at

| | | |
|---|---|---|
| NR | 0.3239007 | 0.6545010 |
| R | −0.5514259 | 0.6035657 |

tables$X235421_at
X235421_at

| | | |
|---|---|---|
| NR | −0.2094653 | 0.4583538 |
| R | 0.4672043 | 0.4014682 |

tables$X235639_at
X235639_at

| | | |
|---|---|---|
| NR | 0.3234035 | 0.5491068 |
| R | −0.3795961 | 0.6104110 |

tables$X235688_s_at
X235688_s_at

| | | |
|---|---|---|
| NR | 0.01982982 | 0.5208595 |
| R | −0.55915444 | 0.5353062 |

tables$X235804_at
X235804_at

| | | |
|---|---|---|
| NR | 0.2009344 | 0.5886169 |
| R | −0.4162556 | 0.4684402 |

tables$X235831_at
X235831_at

| | | |
|---|---|---|
| NR | −0.08120476 | 0.4915252 |
| R | 1.14904526 | 1.1198369 |

tables$X236203_at
X236203_at

| | | |
|---|---|---|
| NR | 0.005259016 | 0.3895193 |
| R | 0.676842217 | 0.4524154 |

tables$X236280_at
X236280_at

| | | |
|---|---|---|
| NR | −0.1220235 | 0.4998530 |
| R | 0.5788924 | 0.4002499 |

tables$X236295_s_at
X236295_s_at

| | | |
|---|---|---|
| NR | −0.1165942 | 0.5645452 |
| R | 0.6163840 | 0.5662135 |

tables$X236583_at
X236583_at

| | | |
|---|---|---|
| NR | 0.02541689 | 0.5675783 |
| R | 0.82576116 | 0.7584443 |

tables$X236908_at
X236908_at

| | | |
|---|---|---|
| NR | 0.5408046 | 0.8859905 |
| R | −0.3849473 | 0.3825884 |

tables$X238439_at
X238439_at

| | | |
|---|---|---|
| NR | −0.08639162 | 0.4532391 |
| R | 0.64076976 | 0.7133665 |

tables$X238488_at
X238488_at

| | | |
|---|---|---|
| NR | −0.2588720 | 0.5056425 |
| R | 0.4974834 | 0.7836170 |

tables$X238544_at
X238544_at

| | | |
|---|---|---|
| NR | 0.2824277 | 0.7145403 |
| R | −0.4079449 | 0.4677199 |

tables$X239196_at
X239196_at

| | | |
|---|---|---|
| NR | 0.04478165 | 0.456084 |
| R | 0.92699218 | 0.784260 |

tables$X239237_at
X239237_at

| | | |
|---|---|---|
| NR | −0.06887682 | 0.4538298 |
| R | 0.56173192 | 0.5087772 |

tables$X239744_at
X239744_at

| | | |
|---|---|---|
| NR | −0.1939947 | 0.4873477 |
| R | 0.3861305 | 0.5051036 |

tables$X241671_x_at
X241671_x_at

| | | |
|---|---|---|
| NR | 0.3853780 | 0.6714183 |
| R | −0.2467927 | 0.4002054 |

tables$X241701_at
X241701_at

| | | |
|---|---|---|
| NR | 0.3603183 | 0.8440397 |
| R | −0.4068724 | 0.5295527 |

tables$X242458_at
X242458_at

| | | |
|---|---|---|
| NR | −0.2312046 | 0.7607719 |
| R | 0.5057350 | 0.7076187 |

tables$X242546_at
X242546_at

| | | |
|---|---|---|
| NR | 0.1097238 | 0.4715672 |
| R | −0.4309505 | 0.5259991 |

tables$X242874_at
X242874_at

| | | |
|---|---|---|
| NR | −0.2219024 | 0.3807166 |
| R | 0.5644292 | 0.7338815 |

tables$X242881_x_at
X242881_x_at

| | | |
|---|---|---|
| NR | 0.08543868 | 0.3930728 |
| R | −0.63018680 | 0.5655045 |

tables$X242986_at
X242986_at

| | | |
|---|---|---|
| NR | 0.2371634 | 0.5409865 |
| R | −0.6643602 | 0.7457416 |

tables$X243099_at
X243099_at

| | | |
|---|---|---|
| NR | 0.01903757 | 0.6357632 |
| R | 0.80481642 | 0.8461258 |

tables$X244023_at
X244023_at

| | | |
|---|---|---|
| NR | −0.3068381 | 0.6806949 |
| R | 0.4666732 | 0.5605710 |

tables$X244061_at
X244061_at

| | | |
|---|---|---|
| NR | −0.2379335 | 0.5919363 |
| R | 0.5615362 | 0.6126814 |

tables$X32128_at
X32128_at

| | | |
|---|---|---|
| NR | −0.04592188 | 0.6445335 |
| R | 0.60003775 | 0.6856519 |

tables$X34210_at
X34210_at

| | | |
|---|---|---|
| NR | −0.3404304 | 0.6066166 |
| R | 0.4975685 | 0.2978426 |

tables$X38149_at
X38149_at

| | | |
|---|---|---|
| NR | −0.4040241 | 0.6171240 |
| R | 0.2427490 | 0.4577025 |

tables$X64064_at
X64064_at

| | | |
|---|---|---|
| NR | −0.4613187 | 0.6447816 |
| R | 0.4548881 | 0.3392856 |

levels
[1] “NR” “R”
call
naiveBayes.default(x = X, y = Y,
laplace = laplace)

1-31. (canceled)

31. A method of designating a patient as having a responder or a non-responder gene profile to Mage cancer immunotherapy comprising the steps of:
a) analyzing a patient derived tumor sample for upregulation of one or more of the following genes: CCL5, TRAT1, STAT4, PRKCQ, GPR171, UBD, CD52, CD3D, PRF1, CD8A, CXCL10, CD69, TRBV19, TRDV2, IL7R, GZMK, and CD45R; and
b) characterizing the patient from which the sample was derived as a having a responder or a non-responder gene profile based on the results of step (a), wherein upregulation of at least one of said genes indicates the patient has a responder gene profile.

32. A kit comprising at least one component for performing an analysis on a patient derived sample to identify a responder according to the method of claim 31.

33. A method of inducing a responder gene profile in a patient characterised as a non-responder comprising the step of stimulating a systemic immune or inflammatory response in the patient by radiotherapy or administering effective amount of interferon.


SEQUENCE LISTING (163 sequences)

SEQ ID NO: 1 - DNA, 4039 bp, Homo Sapien
atacttacaattacgagatttatatttgcattagtctctttggctggtgggtaggggtgagaggctcttcctggatcccttattttctacaggagaggaggaaaacacctgggatgctccagtgctcttacgcagataatgatcattaacatcagcctctctgatcaaagagctactccaccccactctggctgtagtgtgacattcctgcctgcctgaggaagaaatggtgagcaggggctgcaattgcagacaagtgtcacccagaagccacaagtttctgtgagcaccaggtctacaaactacccaaggcatagcaatggcattatcactggaagaattcgtccactcccttgacctcaggaccctacccagggttctagaaatccaggcaggcatctatcttgaaggctctatttatgaaatgtttggaaatgaatgctgtttttcaacaggagaagtgattaaaattactggtctcaaagttaagaagatcatagctgaaatttgtgagcagattgaaggttgtgagtctctacagccatttgaactgcctatgaattttccaggtctttttaagattgtggctgataaaactccataccttactatggaagaaatcacaaggaccattcatattggaccaagtagactagggcatccttgcttctatcatcagaaggatataaaactagagaacctcatcataaagcagggtgagcaaatcatgctcaactcagttgaagagattgatggagaaataatggtgagctgtgcagtagcaaggaatcatcaaactcactcatttaatttgcctttgtcacaagaaggagaattctacgagtgtgaagatgaacgtatttacactctaaaggagattgttgaatggaagattcctaagaacagaacaagaactgtaaaccttacagatttttcaaataagtgggactcaacgaatccatttcctaaagacttttatggtaccctgattctcaagcctgtttatgaaattcaaggtgtgatgaaatttcgaaaagatataatccgcatcctccccagtctagatgtcgaagtcaaagacatcactgattcttacgatgctaactggtttcttcagctgttatcaacagaagatctttttgaaatgactagtaaagagttccccatagtgactgaagtcatagaagcacctgaaggaaaccacctgccccaaagcattttacagcctgggaaaaccattgtgatccacaaaaagtaccaggcatcaagaatcttagcttcagaaattagaagcaattttcctaaaagacacttcttgatccccactagctataaaggcaagttcaagcggcgaccgagggagttcccaacggcctatgacctagagatcgctaagagtgaaaaggagcctcttcacgtggtggccaccaaagcgtttcattcccctcatgacaagctgtcatccgtatctgttggggaccagtttctggtgcatcagtcagagacgactgaagtcctctgtgagggaataaaaaaagtggtgaatgttctggcctgtgaaaaaatcctcaaaaagtcctatgaggctgcgctgctccctttgtacatggaaggaggttttgtagaggtgattcatgataagaaacagtacccgatttctgagctctgtaaacagttccgtttgcccttcaatgtgaaggtgtctgtcagggatctttccattgaagaggacgtgttggctgccacaccaggactgcagttggaggaggacattacagactcttacctactcataagtgactttgccaaccccacggagtgctgggaaattcctgtgggccgcttgaatatgactgttcagttagttagtaatttctctagggatgcagaaccatttctagtcaggactctggtagaagagatcactgaagagcaatattacatgatgcggagatatgaaagctcagcctcacatcccccacctcgccctccgaaacacccctcagtagaggaaacaaagttaaccctgctaaccttagcagaagaaaggacggtagacctgcccaagtctcccaagcgtcatcacgtagacataaccaagaaacttcacccaaatcaagctggcctggattcaaaagtactgattggtagtcagaatgatttggtggatgaagagaaagaaaggagcaaccgtggggccacagcaatagcagaaacattcaaaaatgaaaaacatcaaaaataacaagatgtgacagaagccacttaggcagcaaacataaatgttgcagtgaaaaaagaagctagccttctagctgaaaaacgagtattccccaatggactccagaagaaacttgattcatcgctgcaaaggaaagaacaaccttaaaacttttaacagataaaacttacagaaacctatgatatagaattcatatagtctattctgttgtgtctaaatctgtaggcattgtgttgttgttctttaggacgtatttatttaacttgcacattttttcagattcttatttctactaccaacaactaagtaattgggaaataattctgtatttcagtttctgagtaaaaccagtctgaaataggataaaagccaccaaatattttcttttttttccagaatttgttttgccattttttagtgctatcatcattcctaacaagactaacttacagaaaaataattatatctgactgatttaaaatgttcaggtttcttatccaaatcccttggaactatggaaaggagtttgatttcacattcacagtgtatttacaaaatacgctgtgtcataaatatgtttgaattccaacagccaaagccattgagagtcataggagttttccataaccttctcttctatgacccaacaacaagctcatgactgaaatttcaccagatttctgagacgatgtcttaatattctatgtgctatgtaccagataattctttagatgaatgtttcttaggattgtaggaaaattatctagttaatcataatatttgatggaaagaaaaagacaataaaattgtaatataataaatttggctgacaagaaaccaaagtgattcttaattagtatacatcagaatgatgctcttatagttgtaccatctataaaaattactttaagggctctcacattttaataatttatcttattatgtattaagtatacaggaacaatattatttttcctttaacaaaatgaagagacaggctatctggttaatgttacataggaatttaatagtaatgcttgaacttcatccatagatcatactctgtacaaaatttgttagctaacatcctatctcataattattttatgttttgtggagaaatttgttgattttgtaccaaagtgtttctgaagacaataaattgtgagtcaactttagaacaaaaaaaattagagttttttcaatgtttatattctgattaagcttactttaccttacattttttctaagtaacaatgaatcctgatttctagtgtcctaaaaattgcttagtgatttgattgtggtaatatcatttcttatctacaatgtctaaagttttatgggacagtttttcttttatttattttgcctgtttgtgcagataagaacagaaacttttctaagacccacatttggttattgaaggccacagcgaatcttaacctaacagccttgacaaactgcaccataggtgtttttagactcatataatttgttatttttcaaacaatagtgaataattaatatttttgtttggaatttgagaacaattaaatttgtacttttagtaactaccattctttgattagaaaattaagagaatgcatatcttactttggttgtaaattatcaagggctttctaatagaaatcatatataacatttctaaatataagtcctttcacatactgtgtttccagttgtcttgatattgaaaagtgtaataaacttcatgctcacctattggagatttgggaaggttgaaaataaacttcctaatttttaaaaaaaaaa

SEQ ID NO: 2 - DNA, 2029 bp, Homo Sapien
gtggacgcgaggagccgggcgcttagaacagaggcttgcacaggtggagatgtggaagtctgtagtgggccatgatgtgtctgtttccgtggagacccagggtgatgattgggacacagatcctgactttgtgaatgacatctctgaaaaggagcaacgatggggagccaagaccatcgaggggtctggacgcacagaacacatcaacatccaccagctgaggaacaaagtatcagaggagcatgatgttctcaggaagaaagagatggagtcagggcccaaagcatcccatggctatggaggtcggtttggagtagaaagagaccgaatggacaagagtgcagtgggccatgagtatgttgccgaggtggagaagcactcttctcagacggatgctgccaaaggctttgggggcaagtacggagttgagagggacagggcagacaagtcagcagtcggctttgattataaaggagaagtggagaagcatacatctcagaaagattactctcgtggctttggtggccggtacggggtggagaaggataaatgggacaaagcagctctgggatatgactacaagggagagacggagaaacacgagtcccagagagattatgccaagggctttggtggccagtatggaatccagaaggaccgagtggataagagcgctgtcggcttcaatgaaatggaggccccgaccacagcttataagaagacgacgcccatagaagccgcttctagtggtgcccgtgggctgaaggcgaaatttgagtccatggctgaggagaagaggaagcgagaggaagaggagaaggcacagcaggtggccaggaggcaacaggagcgaaaggctgtgacaaagaggagccctgaggctccacagccagtgatagctatggaagagccagcagtaccggccccactgcccaagaaaatctcctcagaggcctggcctccagttgggactcctccatcatcagagtctgagcctgtgagaaccagcagggaacacccagtgcccttgctgcccattaggcagactctcccggaggacaatgaggagcccccagctctgccccctaggactctggaaggcctccaggtggaggaagagccagtgtacgaagcagagcctgagcctgagcccgagcctgagcccgagcctgagaatgactatgaggacgttgaggagatggacaggcatgagcaggaggatgaaccagagggggactatgaggaggtgctcgagcctgaagattcttctttttcttctgctctggctggatcatcaggctgcccggctggggctggggctggggctgtggctctggggatctcagctgtggctctatatgattaccaaggagagggaagtgatgagctttcctttgatccggacgacgtaatcactgacattgagatggtggacgagggctggtggcggggacgttgccatggccactttggactcttccctgcaaattatgtcaagcttctggagtgactagagctcactgtctactgcaactgtgatttcccatgtccaaagtggctctgcctccaccccctccctattcctgatgcaaatgtctaaccagatgagtttctggacagacttccctctcctgcttcattaagggcttggggcagagacagcatggggaaggaggtccccttccccaagagtcctctctatcctggatgagctcatgaacatttctcttgtgttcctgactccttcccaatgaacacctctctgccaccccaagctctgctctcctcctctgtgagctctgggcttcccagtttgtttacccgggaaagtacgtctagattgtgtggtttgcctcattgtgctatttgcccactttccttccctgaagaaatatctgtgaaccttctttctgttcagtcctaaaattcgaaataaagtgagactatggttcacctgtaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 1451 bp, Homo Sapien
gaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatc

SEQ ID NO: 4 - DNA, 523 bp, Homo Sapien
ctcctggttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcaacataagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagcagtgcccagctgggggtcaataaagttacccttgtacttgcaaaaaaaaaaaaaaaaaaa

SEQ ID NO: 5 - DNA, 1579 bp, Homo Sapien
accaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaacaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactagaaaaaaaaaaaaaaaaaaaaaaaaaaaaaaaaaaaaaaaaaaaaaaaaaaaaaaaaaaaaaaaaa

SEQ ID NO: 6 - DNA, 971 bp, Homo Sapien
gattgcttgaggagagaagtatgtgatcagaaagcattctttgtctattaactcctgcccagcaaaagtgaaagaaaattcatgggagcatgcaagaacaaagagcacagcaaagctggacaaacacagcaatccaggcaggggatttccaactcaactctggtatgtaagctgcatgcaaagtcctttttctgtctctggtttctggccccttgtctgcagagatggctcccaatgcttcctgcctctgtgtgcatgtccgttccgaggaatgggatttaatgacctttgatgccaacccatatgacagcgtgaaaaaaatcaaagaacatgtccggtctaagaccaaggttcctgtgcaggaccaggttcttttgctgggctccaagatcttaaagccacggagaagcctctcatcttacggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctgggcatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttgattctaacacaattaattaagtgacatgatttttactaatgtattactgagactagtaaataaatttttaagccaa

SEQ ID NO: 7 - DNA, 2761 bp, Homo Sapien
agaggacgcccggtgaaggggctccagcctggcagtttctgcgtgttagcatttctagaatagagtgggtgggaactgacccaagtaaagtcccagagactcgaacactgacgcacaggaaagcctcaagtgggaggagaaatgcaaatcccctactgatgatggcgtcagcggctttctcctagggactgtgaggggcgcttctgactttggacttgagcactgcctgggacctgtgctgagagagcgctagcatgtctcagtggaatcaagtccaacagttagaaatcaagtttttggagcaggtggatcaattctatgatgacaactttcccatggaaattcggcatctgttggcccaatggattgaaaatcaagactgggaggcagcttctaacaatgaaaccatggcaacgattcttcttcaaaacttgttaatacaactggatgaacagttaggtcgtgtttccaaagagaaaaacctactcttgatacacaatctaaaaagaattaggaaggtccttcagggaaaatttcatggaaatccaatgcatgtagctgtggttatttcaaactgtttaagggaagagaggagaatattggctgcagccaacatgcctgtccaggggcctctagagaaatccttacaaagttcttcagtttcagaaagacagaggaatgtggagcacaaagtggctgccattaaaaacagtgtgcagatgacagaacaagataccaaatacttagaagatctgcaagacgaatttgactacaggtataaaacaattcagacaatggatcagagtgacaagaatagtgccatggtgaatcaggaagttttgacactgcaggaaatgcttaacagcctcgatttcaagagaaaggaggctctcagtaaaatgacccaaatcatccatgagacagacctgttaatgaacaccatgctcatagaagagctgcaagactggaagcggcggcagcaaatcgcctgcatcgggggtccactccacaatgggctcgaccagcttcagaactgctttacactattggcagaaagtcttttccaactgagaaggcaattggagaaactagaggagcaatctaccaaaatgacatatgaaggtgatcccattccaatgcaaagaactcacatgctagaaagagtcaccttcttgatctacaaccttttcaagaactcatttgtggttgagcgacagccatgtatgccaacccaccctcagaggccgttggtacttaaaaccctaattcagttcactgtaaaactaaggctactaataaaattgccagaactaaactatcaggtaaaggttaaggcatcaattgacaagaatgtttcaactctaagcaaccgaagatttgtactttgtggaactaatgtcaaagccatgtctattgaagaatcttccaatgggagtctctcagtagaatttcgacatttgcaaccaaaggaaatgaagtccagtgctggaggtaaaggaaatgagggctgtcacatggtgactgaagaacttcattccataacgtttgaaacacagatctgcctctatggcctgaccatagatttggagaccagctcattgcctgtggtgatgatttccaatgtcagtcagttacctaatgcttgggcatccatcatttggtacaacgtgtcaaccaacgattcccagaacttggttttctttaataatcctccacctgccacattgagtcaactactggaggtgatgagctggcagttttcatcgtacgttggtcgtggtcttaactcagatcaactccatatgctggcagagaagcttacagtccaatctagctacagtgatggtcacctcacctgggccaagttctgcaaggaacatttacctggtaaatcatttaccttttggacatggcttgaagcaatattggatctaattaagaaacacattcttcccctttggattgatgggtatgtcatgggctttgttagcaaagagaaggaacggctgttgctaaaggataaaatgcctggcacctttttattaagattcagtgaaagccatctcggaggaataactttcacctgggtggaccattctgaaagtggggaagtgagattccactctgtagaaccctacaataaaggccggttgtctgctctgccattcgctgacatcctgcgagactacaaagttattatggctgaaaacattcctgaaaaccctctgaagtacctatatcctgacattcccaaagacaaagccttcggtaaacactacagctctcagccttgcgaagtttcaagaccaacagaaaggggtgacaaaggttatgttccttctgtttttatccccatctcaacaatccgaagtgattcaacagagccacattctccatcagaccttcttcccatgtctccaagtgtgtatgcggtgttgagagaaaacctgagtcccacaacaattgaaactgcaatgaagtctccttattctgctgaatgacaggataaactctgacgcaccaagaaaggaagcaaatgaaaaagtttaaagactgttctttgcccaataaccacattttatttcttcagctttgtaaataccaggttctaggaaatgtttgacatctgaagctctcttcacactcccgtggcactcctcaattgggagtgttgtgactgaaatgcttgaaaccaaagcttcagataaacttgcaagataagacaactttaagaaaccagtgttaataacaatattaacag

SEQ ID NO: 8 - DNA, 1074 bp, Homo Sapien
gatcaacacatttcatctgggcttcttaaatctaaatctttaaaatgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaattaagttacaaataattttattggatgcacttgcttcttttttcctaatatgctcgcaggttagagttgggtgtaagtaaagcagagcacatatggggtccatttttgcacttgtaagtcattttattaaggaatcaagttctttttcacttgtatcactgatgtatttctaccatgctggttttattctaaataaaatttagaagactcaaaaaaaaaaaaaaaaaaaaaaaaaaaaa

SEQ ID NO: 9 - DNA, 822 bp, Homo Sapien
gggctgctccacgcttttgccggagacagagactgacatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattgaactcaggactcagagtagtccaggtgttctcctcctattcagttcccagaatcaaagcaatgcattttggaaagctcctagcagagagactttcagccctaaatctagactcaaggttcccagagatgacaaatggagaagaaaggccatcagagcaaatttgggggtttctcaaataaaataaaaataaaaacaaatactgtgtttcagaagcgccacctattggggaaaattgt

SEQ ID NO: 10 - DNA, 1855 bp, Homo Sapien
ggttgctactgctgcggctaaccaaacagctcatgcttctctgaagacttgcagcaaggtttgctgaggctcacagaagatagccccagtgttttggagtggttttgaatgtgattctgagatcagactgactgagctggaatcctggctttatatcttaccagctacacaaccttggagtcttagaaattttttcttttcaataagcagtcatccttactttccctcaagatgacaaacagttcgttcttctgcccagtttataaagatctggagccattcacgtattttttttatttagttttccttgttggaattattggaagttgttttgcaacctgggcttttatacagaagaatacgaatcacaggtgtgtgagcatctacttaattaatttgcttacagccgatttcctgcttactctggcattaccagtgaaaattgttgttgacttgggtgtggcaccttggaagctgaagatattccactgccaagtaacagcctgcctcatctatatcaatatgtatttatcaattatcttcttagcatttgtcagcattgaccgctgtcttcagctgacacacagctgcaagatctaccgaatacaagaacccggatttgccaaaatgatatcaaccgttgtgtggctaatggtccttcttataatggtgccaaatatgatgattcccatcaaagacatcaaggaaaagtcaaatgtgggttgtatggagtttaaaaaggaatttggaagaaattggcatttgctgacaaatttcatatgtgtagcaatatttttaaatttctcagccatcattttaatatccaattgccttgtaattcgacagctctacagaaacaaagataatgaaaattacccaaatgtgaaaaaggctctcatcaacatacttttagtgaccacgggctacatcatatgctttgttccttaccacattgtccgaatcccgtataccctcagccagacagaagtcataactgattgctcaaccaggatttcactcttcaaagccaaagaggctacactgctcctggctgtgtcgaacctgtgctttgatcctgtcctgtactatcacctctcaaaagcattccgctcaaaggtcactgagacttttgcctcacctaaagagaccaaggctcagaaagaaaaattaagatgtgaaaataatgcataaaagacaggatttttgtgctaccaattctggccttactggaccataaagttaattatagctttgaaagataaaaaaaaaaaaaaaaacaaaaaaaaactcagtatgaaaaaatacagttagctagcaaatatggacaggtttacttagaaatcctgtttctaaatgcaagtcaagctttattgttaggcttgctgctactcattaacccaaatatttgtacaaaaaactaaagagtctcattgaacgaatgtaaaatcctgcaatatccttgaaatccaaaagaggtccatgacatagacccaaaggtattcatgagttattcatttaaatgcctggaactgacttcttgataaaaatataaaaaataatttccatgtaagttaccagaaagcccaccagcaacataattttaaagcctttcggattacttttaaaaaatgcagcttacatataacaacttgtgcctattttatttctaatctatcacttcaaaagatggtaatctttcaactcattattcctccaatttttaatgtcgaatttttttctaacacaataaccaaaagcttttatttataaaaaggcttgaaaaatataaaaaaaaaaaaaaaaaaaaaaaaaaaaaaaaaaaaaaaaaaaaaaaaaaaaaaaaaaaaaaaaaaaa

SEQ ID NO: 11 - DNA, 2109 bp, Homo Sapien
gggggcaacggtcacctgatctgcggctgtcgaggccgctgaggcagtggaggctgaggctatgatggcggccatggcgacggctcgagtgcggatggggccgcgatgcgcccaggcgctctggcgcatgccgtggctgccggtgtttttgtcgttggcggcggcggcggcggcggcagcggcggagcagcaggtcccgctggtgctgtggtcgagtgaccgggacttgtgggctcctgcggccgacactcatgaaggccacatcaccagcgacttgcagctctctacctacttagatcccgccctggagctgggtcccaggaatgtgctgctgttcctgcaggacaagctgagcattgaggatttcacagcatatggcggtgtgtttggaaacaagcaggacagcgccttttctaacctagagaatgccctggacctggccccctcctcactggtgcttcctgccgtcgactggtatgcagtcagcactctgaccacttacctgcaggagaagctcggggccagccccttgcatgtggacctggccaccctgcgggagctgaagctcaatgccagcctccctgctctgctgctcattcgcctgccctacacagccagctctggtctgatggcacccagggaagtcctcacaggcaacgatgaggtcatcgggcaggtcctgagcacactcaagtccgaagatgtcccatacacagcggccctcacagcggtccgcccttccagggtggcccgtgatgtagccgtggtggccggagggctaggtcgccagctgctacaaaaacagccagtatcacctgtgatccatcctcctgtgagttacaatgacaccgctccccggatcctgttctgggcccaaaacttctctgtggcgtacaaggaccagtgggaggacctgactcccctcacctttggggtgcaggaactcaacctgactggctccttctggaatgactcctttgccaggctctcactgacctatgaacgactctttggtaccacagtgacattcaagttcattctggccaaccgcctctacccagtgtctgcccggcactggtttaccatggagcgcctcgaagtccacagcaatggctccgtcgcctacttcaatgcttcccaggtcacagggcccagcatctactccttccactgcgagtatgtcagcagcctgagcaagaagggtagtctcctcgtggcccgcacgcagccctctccctggcagatgatgcttcaggacttccagatccaggctttcaacgtaatgggggagcagttctcctacgccagcgactgtgccagcttcttctcccccggcatctggatggggctgctcacctccctgttcatgctcttcatcttcacctatggcctgcacatgatcctcagcctcaagaccatggatcgctttgatgaccacaagggccccactatttctttgacccagattgtgtgaccctgtgccagtgggggggttgagggtgggacggtgtccgtgttgttgctttcccaccctgcagcgcactggactgaagagcttccctcttcctactgcagcatgaactgcaagctcccctcagcccatcttgctccctcttcagcccgctgaggagctttcttgggctgcccccatctctcccaacaaggtgtacatattctgcgtagatgctagaccaaccagcttcccagggttcgtcgctgtgaggcgtaagggacatgaattctagggtctcctttctccttatttattcttgtggctacatcatccctggctgtggatagtgcttttgtgtagcaaatgctccctccttaaggttatagggctccctgagtttgggagtgtggaagtactacttaactgtctgtcctgcttggctgtcgttatcgttttctggtgatgttgtgctaacaataagaagtacacgggtttatttctgtggcctgagaaggaagggacctccacgacaggtgggctgggtgcgatcgccggctgtttggcatgttcccaccgggagtgccgggcaggagcatggggtgcttggttgtttccttcctaataaaataaacgcgggtcgccatgcaaaaaaaaaa

SEQ ID NO: 12 - DNA, 1267 bp, Homo Sapien
acattctcttttcttttattcttgtctgttctgcctcactcccgagctctactgactcccaacagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatagacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggattgcgcaaaagcaatgcagcagaacgcagggggcctctgtaaggcacatggaggtgatggtgtttcttagagagaagatcactgaagaaacttctgctttaatggctttacaaagctggcaatattacaatccttgacctcagtgaaagcagtcatcttcagcattttccagccctatagccaccccaagtgtggatatgcctcttcgattgctccgtactctaacatctagctggcttccctgtctattgccttttcctgtatctattttcctctatttcctatcattttattatcaccatgcaatgcctctggaataaaacatacaggagtctgtctctgctatggaatgccccatggggcatctcttgtgtacttattgtttaaggtttcctcaaactgtgatttttctgaacacaataaactattttgatgatcttgggtggaaaaaaaaaaaaaaaaa

SEQ ID NO: 13 - DNA, 1190 bp, Homo Sapien
cagatccatcaggtccgagctgtgttgactaccacttttcccttcgtctcaattatgtcttggaaaaaggctttgcggatccccggaggccttcgggcagcaactgtgaccttgatgctgtcgatgctgagcaccccagtggctgagggcagagactctcccgaggatttcgtgtaccagtttaagggcatgtgctacttcaccaacgggacagagcgcgtgcgtcttgtgagcagaagcatctataaccgagaagagatcgtgcgcttcgacagcgacgtgggggagttccgggcggtgacgctgctggggctgcctgccgccgagtactggaacagccagaaggacatcctggagaggaaacgggcggcggtggacagggtgtgcagacacaactaccagttggagctccgcacgaccttgcagcggcgagtggagcccacagtgaccatctccccatccaggacagaggccctcaaccaccacaacctgctggtctgctcggtgacagatttctatccagcccagatcaaagtccggtggtttcggaatgaccaggaggagacagctggcgttgtgtccaccccccttattaggaatggtgactggaccttccagatcctggtgatgctggaaatgactccccagcgtggagacgtctacacctgccacgtggagcaccccagcctccagagccccatcaccgtggagtggcgggctcaatctgaatctgcccagagcaagatgctgagtggcattggaggcttcgtgctggggctgatcttcctcgggctgggccttatcatccatcacaggagtcagaaagggctcctgcactgactcctgagactattttaactgggattggttatcacttttctgtaacgcctgcttgtccctgcccagaattcccagctgtctgtgtcagcctgtccccctgagatcagagtcctacagtggctgtcacgcagccaccaggtcatctcctttcatccccaccttgaggcggatggctgtgaccctacttcctgcactgacccacagcctctgcctgtgcacggccagctgcatctactcaggccccaaggggtttctgtttctattctctcctcagactgctcaagagaagcacatgaaaaccattacctgactttagagcttttttacataattaaacatgatcctgagtt

SEQ ID NO: 14 - DNA, 2666 bp, Homo Sapien
atgcactcaagcagagaagaaatccacaaagactcacagtctgctggtgggcagagaagacagaaacgacatgagcacagcaggaaaagtaatcaaatgcaaagcagctgtgctatgggaggtaaagaaacccttttccattgaggatgtggaggttgcacctcctaaggcttatgaagttcgcattaagatggtggctgtaggaatctgtcacacagatgaccacgtggttagtggcaacctggtgaccccccttcctgtgattttaggccatgaggcagccggcatcgtggagagtgttggagaaggggtgactacagtcaaaccaggtgataaagtcatcccgctctttactcctcagtgtggaaaatgcagagtttgtaaaaacccggagagcaactactgcttgaaaaatgatctaggcaatcctcgggggaccctgcaggatggcaccaggaggttcacctgcagggggaagcccattcaccacttccttggcaccagcaccttctcccagtacacggtggtggatgagaatgcagtggccaaaattgatgcagcctcgcccctggagaaagtctgcctcattggctgtggattctcgactggttatgggtctgcagttaacgttgccaaggtcaccccaggctctacctgtgctgtgtttggcctgggaggggtcggcctatctgctgttatgggctgtaaagcagctggagcagccagaatcattgcggtggacatcaacaaggacaaatttgcaaaggccaaagagttgggtgccactgaatgcatcaaccctcaagactacaagaaacccatccaggaagtgctaaaggaaatgactgatggaggtgtggatttttcgtttgaagtcatcggtcggcttgacaccatgatggcttccctgttatgttgtcatgaggcatgtggcacaagcgtcatcgtaggggtacctcctgcttcccagaacctctcaataaaccctatgctgctactgactggacgcacctggaagggggctgtttatggtggctttaagagtaaagaaggtatcccaaaacttgtggctgattttatggctaagaagttttcactggatgcgttaataacccatgttttaccttttgaaaaaataaatgaaggatttgacctgcttcactctgggaaaagtatccgtaccgtcctgacgttttgaggcaatagagatgccttcccctgtagcagtcttcagcctcctctaccctacaagatctggagcaacagctaggaaatatcattaattcagctcttcagagatgttatcaataaattacacatgggggctttccaaagaaatggaaattgatgggaaattatttttcaggaaaatttaaaattcaagtgagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagttttcactgtcgacattttcctttttctaataaaaatgtaccaaatccctggggtaaaagctagggtaaggtaaaggatagactcacatttacaagtagtgaaggtccaagagttctaaatacaggaaatttcttaggaactcaaataaaatgccccacattttactacagtaaatggcagtgtttttatgacttttatactatttctttatggtcgatatacaattgattttttaaaataatagcagatttcttgcttcatatgacaaagcctcaattactaattgtaaaaactgaactattcccagaatcatgttcaaaaaatctgtaatttttgctgatgaaagtgcttcattgactaaacagtattagtttgtggctataaatgattatttagatgatgactgaaaatgtgtataaagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaagacctgtgttaatgctttgttaatgcttttcccactctcatttgttaatgctttcccactctcaggggaaggatttgcattttgagctttatctctaaatgtgacatgcaaagattattcctggtaaaggaggtagctgtctccaaaaatgctattgttgcaatatctacattctatttcatattatgaaagaccttagacataaagtaaaatagtttatcatttactgtgtgatcttcagtaagtctctcaggctctctgagcttgttcatcctttgttttgaaaaaattactcaaccaatccattacagcttaaccaagattaaatgggatgatgttaatgaaagagcttcgcc

SEQ ID NO: 15 - DNA, 1508 bp, Homo Sapien
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgaaggagccctggttctgctgaggtgcaactactcatcgtctgttccaccatatctcttctggtatgtgcaataccccaaccaaggactccagcttctcctgaagtacacatcagcggccaccctggttaaaggcatcaacggttttgaggctgaatttaagaagagtgaaacctccttccacctgacgaaaccctcagcccatatgagcgacgcggctgagtacttctgtgctgtgagtgatctcgaaccgaacagcagtgcttccaagataatctttggatcagggaccagactcagcatccggccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcctacccccaaggaggtgaaagctgctaccacctctgtgcccccccggtaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgctgccgatgccttcattaaaatgatttggaa

SEQ ID NO: 16 - DNA, 1702 bp, Homo Sapien
agactcaacaagagctccagcaaagactttcactgtagcttgacttgacctgagattaactagggaatcttgagaataaagatgagctctgaaaattgtttcgtagcagagaacagctctttgcatccggagagtggacaagaaaatgatgccaccagtccccatttctcaacacgtcatgaagggtccttccaagttcctgtcctgtgtgctgtaatgaatgtggtcttcatcaccattttaatcatagctctcattgccttatcagtgggccaatacaattgtccaggccaatacacattctcaatgccatcagacagccatgtttcttcatgctctgaggactgggttggctaccagaggaaatgctactttatttctactgtgaagaggagctggacttcagcccaaaatgcttgttctgaacatggtgctactcttgctgtcattgattctgaaaaggacatgaactttctaaaacgatacgcaggtagagaggaacactgggttggactgaaaaaggaacctggtcacccatggaagtggtcaaatggcaaagaatttaacaactggttcaacgttacagggtctgacaagtgtgtttttctgaaaaacacagaggtcagcagcatggaatgtgagaagaatttatactggatatgtaacaaaccttacaaataataaggaaacatgttcacttattgactattatagaatggaactcaaggaaatctgtgtcagtggatgctgctctgtggtccgaagtcttccatagagactttgtgaaaaaaaattttatagtgtcttgggaattttcttccaaacagaactatggaaaaaaaggaagaaattccaggaaaatctgcactgtgggcttttattgccatgagctagaagcatcacaggttgaccaataaccatgcccaagaatgagaagaatgactatgcaacctttggatgcactttatattattttgaatccagaaataatgaaataactaggcgtggacttactatttattgctgaatgactaccaacagtgagagcccttcatgcatttgcactactggaaggagttagatgttggtactagatactgaatgtaaacaaaggaattatggctggtaacataggtttttagtctaattgaatcccttaaactcagggagcatttataaatggacaaatgcttatgaaactaagatttgtaatatttctctctttttagagaaatttgccaatttactttgttatttttccccaaaaagaatgggatgatcgtgtatttatttttttacttcctcagctgtagacaggtccttttcgatggtacatatttctttgcctttataatcttttatacagtgtcttacagagaaaagacataagcaaagactatgaggaatatttgcaagacatagaatagtgttggaaaatgtgcaatatgtgatgtggcaaatctctattaggaaatattctgtaatcttcagacctagaataatactagtcttataataggtttgtgactttcctaaatcaattctattacgtgcaatacttcaatacttcatttaaaatatttttatgtgcaataaaatgtatttgtttgtattttgtgttcagtacaattataagctgtttttatatatgtgaaataaaagtagaataaacacaaaaaaaaaaaaaaaaaaaaaaaaaaaa

SEQ ID NO: 17 - DNA, 3273 bp, Homo Sapien
agtccccgcgcagtccccgcgcagtcccagcgccaccgggcagcagcggcgccgtgctcgctccagggcgcaaccatgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggccgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacattccagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaaccccgggatggagcggctgatatcctgaatcttgcccctccagagacaggaaagaatttgccttctccctgggaactggttcaagagacactgcttgggttcctttttcaacttggaaaaagaaagaaacactcaacaataaagactgagacccgttcgcccccatgtgacttttatctgtagcagaaaccaagtctacttcactaatgacgatgccgtgtgtctcgtctcctgacatgtctcacagacgctcctgaagttaggtcattactaaccatagttatttacttgaaagatgggtctccgcacttggaaaggtttcaagacttgatactgcaataaattatggctcttcacctgggcgccaactgctgatcaatgaaatgcttgttgaatcaggggcaaacggagtacagacgtctcaagactgaaacggccccattgcctggtctagtagcggatctcactcagccgcagacaagtaatcactaacccgttttattctattcctatctgtggatgtgtaaatggctggggggccagccctggataggtttttatgggaattctttacaataaacatagcttgtaacttgagatctacaaatccattcatcctgattgggcatgaaatccatggtcaagaggacaagtggaaagtgagagggaaggtttgctagacaccttcgcttgttatcttgtcaagatagaaaagatagtatcatttcacccttgccagtaaaaacctttccatccacccattctcagcagactccagtattggcacagtcactcactgccattctcacactataacaagaaaagaaatgaagtgcataagtctcctgggaaaagaaccttaaccccttctcgtgccatgactggtgatttcatgactcataagcccctccgtaggcatcattcaagatcaatggcccatgcatgctgtttgcagcagtcaattgagttgaattagaattccaaccatacattttaaaggtatttgtgctgtgtgtatattttgataaaatgttgtgacttcatggcaaacaggtggatgtgtaaaaatggaataaaaaaaaaaaaagagtcaaaaaaaaaaaaa

SEQ ID NO: 18 - DNA, 1074 bp, Homo Sapien
gtaaagctcccatcctgccctgaccctgccatgggcaccagcctcctctgctggatggccctgtgtctcctgggggcagatcacgcagatactggagtctcccagaaccccagacacaacatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagcagggttgaatcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggtatcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccatgtatgctgtgctggtcagcgcccttgtgttgatggccatggtcaagagaaaggatttctgaaggcagccctggaagtggagttaggagcttctaacccgtcatggtttcaatacacattcttcttttgccagcgcttctgaagagctgctctcacctctctgcatcccaata

SEQ ID NO: 19 - DNA, 1080 bp, Homo Sapien
tccaaaatgaagggtctgtggaaggacatgaataaagcacaggaggttgaagtcagatttgcagctttctaggcaggagacaagacaatctgcatcttcacaggagggatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaaagggggccggcactgccagtaaactcacctttgggactggaacaagacttcaggtcacgctcgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccctcgctccttcctctgcattgcccctcttctccctctccaaacagagggaactctcccacccccaaggaggtgaaagctgctaccaccctctgtgcc

SEQ ID NO: 20 - DNA, 1586 bp, Homo Sapien
gaatcaggaagaccagctcctcctactgtcttctgtgttacgggatcagcgttccttgttgagtgggacctgagttttgagagggtcttctgctcctcttggtctggtcccttacttccaagagccccagagaggaaggcatgctgttggctctagctctgcttctagctttcctgcctcctgccagtcagaaatcttccaacttggaagggagaacaaagtcagtcaccaggccaactgggtcatcagctgtaatcacttgtgatcttcctgtagaaaatgccgtctacacccactggtacctacaccaggaggggaaggccccacagcgtcttctgtactatgactcctacaactccagggttgtgttggaatcaggaatcagtcgagaaaagtatcatacttatgcaagcacagggaagagccttaaatttatactggaaaatctaattgaacgtgactctggggtctattactgtgccacctggaaggattattataagaaactctttggcagtggaacaacacttgttgtcacagataaacaacttgatgcagatgtttcccccaagcccactatttttcttccttcgattgctgaaacaaaactccagaaggctggaacatatctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaagacagttattcaaaagatgcaaatgatgtcaccacagtggatcccaaatacaattattcaaaggatgcaaatgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccctgaagcagtcttctttgctagttgaattatgtggtgtgtttttccgtaataagcaaaataaattt

SEQ ID NO: 21 - DNA, 942 bp, Homo Sapien
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtttcccccggcagccgtcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggtttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgctgtgctggtcagtgccctcgtgctgatggccatggtcaagagaaaggattccagaggctga

SEQ ID NO: 22 - DNA, 840 bp, Homo Sapien
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagtgcatactctggggctgggagttaccaactcactttcgggaaggggaccaaactctcggtcataccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtccagctga

SEQ ID NO: 23 - DNA, 671 bp, Homo Sapien
tgcctggccacaggcttcttccccgaccacgtggagctgagctggtgggtgaatgggaaggaggtgcacaatggggtcaacacagacccgcagcccctcaaagaacagcccgccctcaatgactccaaatamtgcctgagcagccgcctgagggtctcggccaccttctggcagaacccccgcaaccacttccgctgtcaagtccagttctacgggctctcggagaatgacgagtggacccaggatagggccaaacccgtcacccaaatcgtcagcgccgaggcctggggtaragcaramtgtggctttacctcggtgtcctaccaacaaggggtcctgtctgccaccatcctctatgaratcctgctagggaaggccaccctgtatgctgtgctggtcagcgcccttgtgttgatggccatggtcaagagaaaggatttctgaaggcagccctggaagtggaattaagaacttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtgactaaaccaataaaaatgttctggtctggcctga

SEQ ID NO: 24 - DNA, 771 bp, Homo Sapien
agagaagcagacatcttctagttcctcccccactctcctctttccggtacctgtgagtcagctaggggagggcagctctcacccaggctgatagttcggtgacctggctttatctactggatgagttccgctgggagatggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtaccttcccttcttgctcagccaataaatatatcctctttcactcagaaaaaaaaaaaaaaaaaaaaaaaaaaaaaa

SEQ ID NO: 25 - DNA, 1421 bp, Homo Sapien
ggcatgcggtgggccctactggtgcttctagctttcctgtctcctgccagtcagaaatcttccaacttggaagggagaacgaagtcagtcaccaggcagactgggtcatctgctgaaatcacttgcgatcttactgtaacaaataccttctacatccactggtacctacaccaggaggggaaggccccacagcgtcttctgtactatgacgtctccactgcaagggatgtgttggaatcaggactcagtccaggaaagtattatactcatacacccaggaggtggagctggatattgagactgcaaaatctaattgaaaatgattctggggtctattactgtgccacctgggacaggcaaaaattattataagaaactctttggcagtggaacaacacttgttgtcacagataaacaacttgatgcagatgtttcccccaagcccactatttttcttccttcaattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaagacagttattcaaaagatgcaaatgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccctgaagcagtcttctttgctagttgaattatgtggtgtgtttttccgtaataagcaaaataaatttaaaaaaatgaaaa

SEQ ID NO: 26 - DNA, 1834 bp, Homo Sapien
gtcaaaggaataatcccatctaaaaagacgaaacagaaagaagtgtatcctgctacacctgcatgcaccccaagcaaccgtctcacagctaaaggagcagaggagactcttggacctcaggtaagcttcaggaagaggagcaggcttcaagtctcacagtggaagctctgctgtggctgttccactcaatctgtccagcaggcagttatttcttcatatgtttcccatcaagtttcagatttatcaaattacataataattgatcatctttctgcaaggcaacaagttaaacgctttagtaaacataatgtaaatatacataaaataaatataatattttcatctccaatagagaaggatgttaacttgagagtcagataaaaaaacgtttgcctatgtttacaaaagcctagtttcttaactgcaagtcagcatatcccaaaacacaagtaattaaggaatgatgtgtgttactttctctgctccctttttaaaaatgaaaccatctatgccatgttctttcaattggcctggggatgtacttaagtttccaagaaaaacaatttatatacaataaatatattaccttgtaatgaaaatgtgctctgcttcatttgacactgaaagtaattaacaagaaaaataaactacttgtagaaaagaaaaaaaccatctgaagaagagactggaaccaaaaggagtaagatgtccaaagagcagactcggccttcctgctctgcaggagccagcacgtccacagccatgggccgttccccacctccccagacctcatcatcagctccacccaacacttcctcaactgaggtacactcttcctggtccccttttgattcattttcttcaacccaaaatgtaggaatctgatttcatcttctactgaaaaatgacatcaatcatcagccagtaaatcaaatgtatagactgagaattaactgcattttaatcttttgcttccacaggcatatttgatgaacttgacattatctctgactgcaggaagttttctgtcctgtgctgtttggggaagagacagagaactgcggaatctggaactttcagcaacagactcactgtctactgcccccatctattatacacccattccctttgctcactaatttgttcaagtttctctgacatacaccatgctcctttttcctttaggattttcacacaccatatttctttcacctttaaactcttacctggccaaccctatccaccctctggatcccaatattgagatcttatcctcagggaatcctcacttagacccctgtaacaggttaaatcttcatggtgttctgtttcctaggaacttctttcttttctactgtttatgacaactgaagttaataagtgtttatctttcccacctactcaaagtagttccaagattagggctagtttgtaattctgtggaccactgtaaacgagggcctagttcagtgtctgcctcatgggaagcttccaataaatacctttgctcaacgaaaaaatgaaaacccagtggctcacgcctgtaatcccagcactttgggaggccgaggcaggtggattgcctgaggtcaggagtttgagactagtctggccaacatggtgaaacactatctctactaaaaatacaaaaaaattagctgggtatggtggcttacgcctataatcccagctactcaggaggctgaggcaggggaattgcttgaaccagggaggtggaggttgcagtgagctgagatcgcaccactgcactccagcctgggtgacagagcgagactccatctcaaaataaaaaagtaaaaaaaaaaaaaaaaaa

SEQ ID NO: 27 - DNA, 1695 bp, Homo Sapien
gaggcacagataaagataagttttactgtcatgctgcttttaacataacagagcaacatcacctaggaaaaaagtttgtaggaggatttttaatccatatatttgtcttatggctagataaagatttctctgaaaaaaagaagcatgtcaggaatctctgggtgcccctttttcctctggggacttctagcattgttgggcttggctttggttatatcactgatcttcaatatttcccactatgtggaaaagcaacgacaagataaaatgtacagctactccagtgaccacaccagggttgatgagtattatattgaagacacaccaatttatggtaacttagatgatatgatttcagaaccaatggatgaaaattgctatgaacaaatgaaagcccgaccagagaaatctgtaaataagatgcaggaagccaccccatctgcacaggcaaccaatgaaacacagatgtgctacgcctcacttgatcacagcgttaaggggaagcgtagaaagcccaggaaacagaatactcatttctcagacaaggatggagatgagcaactacatgcaatagatgccagcgtttctaagaccaccttagtagacagtttctccccagaaagccaggcagtagaggaaaacattcatgatgatcccatcagactgtttggattgatccgtgctaagagagaacctataaactagctggaccatgatctagttcaatgatttggctcctattgaagatggcttctaagaaaacaagatgcacagaggacacagaaggacttggcagcagggtgatgacctgatcatttgttgatgggatggtggcttacctcttattcacagcttacacttatgcatgccaaatgtaaggccatgaaaatcagtatttcaaataacttaaaaaatgctttactactaaaatgtaaaaaattaatgtgctcacctcggcagcacatatactaaaaattaataagacccagcttgaaaattgagcctgataacaagattacaaattcacaatacctaatacttagggaaatataaaaatttaagcatgaatgcgttctggaacacgttagaagaaaaataaaagccaatgagtttttttttaattctcctttctcaccaatgggcaatagcccataattgaaataaatttctgattgaaaggtataggaaacattaaaatgcattactaagagaagtaatataattttcttacaaagtatttttcccaaagatagctttactatttcaaaaattgtcaaattaatgcatgctccttacaacaaacaaatatcaaaaagagtttaggaattctactagccagagatagtcacttggagaaactttctatatatccttctaaatatttttctgggcatgcttatgtatgtacatcagttgtttctttttattttgaaccaaaaatgtggtttcttttgtacacattacttaaactttctttccagtcaacaatatattgtggatttattttcactgttatatttaactatatataaatacgcatatattgtaattttaatgtctgcttagcaccccactgataaccaaatcacagtttatttaaataattttaatgacttttcaaaaacaatttattgatgcaaaaagcaaggttgagatgacaatgtttctttcaataattaaaaaatactgcttcac

SEQ ID NO: 28 - DNA, 2672 bp, Homo Sapien
cttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctatagaaatgtaaatgcaaggtcacacatattaatgacagcctgttgtattaatgatggctccaggtcagtgtctggagtttcattccatcccagggcttggatgtaaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacgtgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaacacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcaaaaaaaaaaaaaaaa

SEQ ID NO: 29 - DNA, 539 bp, Homo Sapien
taaaacagctacaatattccagggccagtcacttgccatttctcataacagcgtcagagagaaagaactgactgaaacgtttgagatgaagaaagttctcctcctgatcacagccatcttggcagtggctgttggtttcccagtctctcaagaccaggaacgagaaaaaagaagtatcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataaacctggtcacctgaaattgaaattgagccacttccttgaagaatcaaaattcctgttaataaaagaaaaacaaatgtaattgaaatagcacacagcattctctagtcaatatctttagtgatcttctttaataaacttgaaagcaaagattttggtttcttaatttccacaaa

SEQ ID NO: 30 - DNA, 1542 bp, Homo Sapien
acaattactctacagctcagaacaccaactgctgaggctgccttgggaagaggatgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtgaatcccatcctggaagggaaggtgcatcgccatctacaggagcagaagaatggacttgctaaatgacctagcactattctctggcccgatttatcatatcccttttctcctccaaatatttctcctctcaccttttctctgggacttaagctgctatatcccctcagagctcacaaatgcctttacattctttccctgacctcctgattttttttttcttttctcaaatgttacctacaaagacatgcctggggtaagccacccggctacctaattcctcagtaacctccatctaaaatctccaaggaagcaataaattccttttatgagatctatgtcaaatttttccatctttcatccagggctgactgaaactatggctaataattggggtactcttatgtttcaatccaatttaacctcatttcccagatcatttttcatgtccagtaacacagaagccaccaagtacagtatagcctgataatatgttgatttcttagctgacattaatatttcttgcttccttgtgttcccacccttggcactgccacccacccctcaattcaggcaacaatgaaattaatggataccgtctgcccttggcccagaattgttatagcaaaaattttagaaccaaaaaataagtctgtactaatttcaatgtggcttttaaaagtatgacagagaaataagttaggataaaggaaatttgaatctca

SEQ ID NO: 31 - DNA, 459 bp, Homo Sapien
acaattactctacagctcagaacaccaactgctgaggctgccttgggaagaggatgatccagcggccgccctttttttttttttttgaatgtttttatatttttatgtttttctctctcaccacattagagattcacatgtcaaaaaaaaagttcaaagattccacttaaatggtagtcaaaattaaaagcatgttgtttaatttgccaactattccataaatcctgtgcttgaagcactaccttttatcaaccacttgaaaggaaggaatatgaagtgtgactcttgaggattgcagttccatttctccacagacatagcagggtgtgtagttccaaatgggtttcttccaaagtcatctcctcagagtaggactgtctgagtagtcattctgataatgttgccattaactacaagcacatctgagaaccctcgtaaggaagtgccattagattcaga

SEQ ID NO: 32 - DNA, 426 bp, Homo Sapien
ggcccagccctaggagggggcagtcccacagcaggctcacaccctgcccttcagagttgcagcagctaatgctgatgccaggaccttgtcctgccaggaagcctctgattagggctgtgaaccaacttccttttcacatctgcaaggactctgctctaagctcaaattttgcagtttctgctgtcatcacagctgggaatgagaaagggaaggggctccagttggtctccctctgcctctggagcacacgtggaggacatgaggcttccgcaggaacagctctgaaagctgagttctcgggtccctgtgaaggagggaagcttgcctgccttggagcgtgaaaaagtcttgctggacagaagtctcacggtttaaaactgggaagggagaggattgagatcacactagaatgcacagaaaattctt

SEQ ID NO: 33 - DNA, 649 bp, Homo Sapien
atggttagtaatttcttccatgtcatacaagtattcgagaaatctgctaccttgattagtaagactgaacacattggttttgtcatttattcatggaggaagtccaccacccacttggggagcagaaggaaatttgccatctcaatttacttatcagaagtttctttgcagaaatatgattgtcccttcagtgggacatcatttgtggtcttctctctctttttgatctgtgcaatggctggagatgtagtctacgctgacatcaaaactgttcggacttccccgttagaactcgcgtttccacttcagagatctgtttctttcaacttttctactgtccataaatcatgtcctgccaaagactggaaggtgcataagggaaaatgttactggattgctgaaactaagaaatcttggaacaaaagtcaaaatgactgtgccataaacaattcatatctcatggtgattcaagacattactgctatggtgagatttaacatttagaggtgacagcatcccccacactggcagtgaattttttgtgctacaaacttggcaaaagtctgtgaaaagaagtttcaacttcatgtgttattaactatacaaatattagttgaatgaattgttgaattacaaaaaaaaaaaaaaa

SEQ ID NO: 34 - DNA, 1353 bp, Homo Sapien
atggatccaaaaactttagccctttctttattagcagctggcgtactagcaggttgtagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggatctggaacagcgtagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgaattggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagggcggtcatcaccatcaccatcaccattaa

SEQ ID NO: 35 - protein, 450 aa, Artificial Sequence
  Fusion Protein
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

SEQ ID NO: 36 - protein, 9 aa, Unknown
  Mage peptide
EVDPIGHLY

SEQ ID NO: 37 - protein, 9 aa, unknown
  mage peptide
FLWGPRALV

SEQ ID NO: 38 - protein, 10 aa, mage peptide
MEVDPIGHLY

SEQ ID NO: 39 - protein, 10 aa, Unknown
  mage peptide
VHFLLLKYRA

SEQ ID NO: 40 - protein, 10 aa, Unknown
  mage peptide
LVHFLLLKYR

SEQ ID NO: 41 - protein, 10 aa, unknown
  mage peptide
LKYRAREPVT

SEQ ID NO: 42 - protein, 16 aa, unknown
  mage peptide
ACYEFLWGPRALVETS

SEQ ID NO: 43 - protein, 12 aa, unknown
  mage peptide
TQHFVQENYLEY

SEQ ID NO: 44 - DNA, 20 bp, Bacterial
tccatgacgttcctgacgtt

SEQ ID NO: 45 - DNA, 18 bp, Bacerial
tctcccagcgtgcgccat

SEQ ID NO: 46 - DNA, 30 bp, Bacterial
accgatgacgtcgccggtgacggcaccacg

SEQ ID NO: 47 - DNA, 24 bp, Bacterial
tcgtcgttttgtcgttttgtcgtt

SEQ ID NO: 48 - DNA, 20 bp, Bacterial
tccatgacgttcctgatgct

SEQ ID NO: 49 - DNA, 347 bp, Unknown
  Probe
  misc_feature (1)...(347): n = A,T,C or G
  misc_feature 132, 149: n = A,T,C or G
ttctaagacccacatttggttattgaaggccacagcgaatcttaacctaacagccttgacaaactgcaccataggtgtttttagactcatataatttgttatttttcaaacaatagtgaataattaatattnttgtttggaatttgagnacaattaaatttgtacttttagtaactaccattctttgattagaaaattaagagaatgcatatcttactttggttgtaaattatcaagggctttctaatagaaatcatatataacatttctaaatataagtcctttcacatactgtgtttccagttgtcttgatattgaaaagtgtaataaacttcatgctcacctat

SEQ ID NO: 50 - DNA, 388 bp, unknown
  probe
ttgctggcttctcttatactaacccagagtttgtcattaatgtgtaggtgaatgcaaactccatcgttgagcctggggtgtaagacttcaagccaagcgtatgtatcaattctagtcttccaggattcacggtgcacatgctggcattcaacatgtggaaagcttgtcttagaggcctttcttgtatgtgtagcttgctagtttgttttctacatttgaaaatgtttagtttagaataagcgcattatccaattatagaggtacaattttccaaacttccagaaactcatcaaatgaacagacaatgtcaaaactactgtgtctgataccaaaatgcttcagtatttgtaatttttcaagtcagaagctgatgttcctggtaaaagtt

SEQ ID NO: 51 - DNA, 411 bp, Unknown
  probe
ttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcacc

SEQ ID NO: 52 - DNA, 388 bp, Unknown
  probe
acagccacgaagatcctaccaaaatgaagcgcttcctcttcctcctactcaccatcagcctcctggttatggtacagatacaaactggactctcaggacaaaacgacaccagccaaaccagcagcccctcagcatccagcagcatgagcggaggcattttccttttcttcgtggccaatgccataatccacctcttctgcttcagttgaggtgacacgtctcagccttagccctgtgccccctgaaacagctgccaccatcactcgcaagagaatcccctccatctttgggaggggttgatgccagacatcaccaggttgtagaagttgacaggcagtgccatgggggcaacagccaaaataggggggtaatgatgtaggggccaagc

SEQ ID NO: 53 - DNA, 428 bp, Unknown
  probe
agacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgc

SEQ ID NO: 54 - DNA, 508 bp, Unknown
  probe
gatcttaaagccacggagaagcctctcatcttatggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatcttattgtattggagggtgaccaccctggggatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttga

SEQ ID NO: 55 - DNA, 501 bp, Unknown
  probe
gctgacatcctgcgagactacaaagttattatggctgaaaacattcctgaaaaccctctgaagtacctatatcctgacattcccaaagacaaagccttcggtaaacactacagctctcagccttgcgaagtttcaagaccaacagaaaggggtgacaaaggttatgttccttctgtttttatccccatctcaacaatccgaagtgattcaacagagccacattctccatcagaccttcttcccatgtctccaagtgtgtatgcggtgttgagagaaaacctgagtcccacaacaattgaaactgcaatgaagtctccttattctgctgaatgacaggataaactctgacgcaccaagaaaggaagcaaatgaaaaagtttaaagactgttctttgcccaataaccacattttatttcttcagctttgtaaataccaggttctaggaaatgtttgacatctgaagctctcttcacactcccgtggcactcctcaattgggag

SEQ ID NO: 56 - DNA, 489 bp, Unknown
  probe
aaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgtcccgcctcatacaaattaagttacaaataattttattggatgcacttgcttcttttttcctaatatgctcgcaggttagagttgggtgtaagtaaagcagagcacatatggggtccatttttgcacttgta

SEQ ID NO: 57 - DNA, 453 bp, Unknown
  probe
gtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattgaactcaggactcagagtagtccaggtgttctcctcctattcagttcccagaatcaaagcaatgcattttggaaagctcctagcagagagactttcagccctaaatctagactcaaggttcccagagatgac

SEQ ID NO: 58 - DNA, 405 bp, Unknown
  probe
ttgccttgtaattcgacagctctacagaaacaaagataatgaaaattacccaaatgtgaaaaaggctctcatcaacatacttttagtgaccacgggctacatcatatgctttgttccttaccacattgtccgaatcccgtataccctcagccagacagaagtcataactgattgctcaaccaggatttcactcttcaaagccaaagaggctacactgctcctggctgtgtcgaacctgtgctttgatcctatcctgtactatcacctctcaaaagcattccgctcaaaggtcactgagacttttgcctcacctaaagagaccaaggctcagaaagaaaaattaagatgtgaaaataatgcataaaagacaggattttttgtgctaccaattctggccttactgga

SEQ ID NO: 59 - DNA, 542 bp, Unknown
  probe
tgatgagctttcctttgatccggacgacgtaatcactgacattgagatggtggacgagggctggtggcggggacgttgccatggccactttggactcttccctgcaaattatgtcaagcttctggagtgactagagctcactgtctactgcaactgtgatttcccatgtccaaagtggctctgctccaccccctccctattcctgatgcaaatgtctaaccagatgagtttctggacagacttccctctcctgcttcattaagggcttggggcagagacagcatggggaaggaggtccccttccccaagagtcctctctatcctggatgagctcatgaacatttctcttgtgttcctgactccttcccaatgaacacctctctgccaccccaagctctgctctcctcctctgtgagctctgggcttcccagtttgtttacccgggaaagtacgtctagattgtgtggtttgcctcattgtgctatttgcccactttccttccctgaagaaatatctgtgaaccttctttctgttcagtccta

SEQ ID NO: 60 - DNA, 101 bp, Unknown
  probe
cgatcaccaatgtacctccagaggtaactgtgctcacgaacagccctgtggaactgagagagcccaacgtcctcatctgtttcatagacaagttcacccca

SEQ ID NO: 61 - DNA, 342 bp, Unknown
  probe
aaaaaaggaaatcgctgcagaatgaaggaatatcccttgaggtgacccagccaacctgtggccagaaggagggttgtaccttgaaaagaccactgaaagcattttggggtgtcaagtaagggtgggcagaggaggtagaaaatcaattcaattgtcgcatcattcatggttctttaatattgatgctcagtgcattggccttagaatatcccagcctctcttctggtttggtgagtgctgtgtaagtaagcatggtagaattgtttggagacatatatagtgatccttggtcactggtgtttcaaacattctggaaagtcacatcgatcaagaatatttttt

SEQ ID NO: 62 - DNA, 555 bp, Unknown
  probe
gcagatttcttgcttcatatgacaaagcctcaattactaattgtaaaaactgaactattcccagaatcatgttcaaaaaatctgtaatttttgctgatcgaaagtgcttcattgactaaacagtattagtttgtggctataaatgattatttagatgatgactgaaaatgtgtataaagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaacacctgtgttaatgctttgttaatgcttttcccactctcatttgttaatgctttcccactctcgggaaggatttgcattttgagctttatctctaaatgtgacatgca

SEQ ID NO: 63 - DNA, 372 bp, Unknown
  probe
caactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctggagaatgttgtgaggggtttatttttttttaatagtgttcataaagaaatacatagtattcttcttctcaagacgtggggggaaattatctcattatcgaggccctgctatgctgtgtgtctgggcgtgttgtatgtcctgc

SEQ ID NO: 64 - DNA, 432 bp, Unknown
  probe
tagtctaattgaatcccttaaactcagggagcatttataaatggcaaatgcttatgaaactaagatttgtaatatttctctctttttagagaaatttgccaatttactttgttatttttccccaaaaagaatgggatgatcgtgtatttatttttttacttcctcagctgtagacaggtccttttcgatggtacatatttctttgcctttataatcttttatacagtgtcttacagagaaaagacataagcaaagactatgaggaatatttgcaagacatagaatagtgttggaaaatgtgcaatatgtgatgtggcaaatctctattaggaaatattctgtaatcttcagacctagaataatactagtcttataataggtttgtgactttcctaaatcaattctattacgtgcaatacttcaatacttcat

SEQ ID NO: 65 - DNA, 352 bp, Unknown
  probe
aatccattcatcctgattgggcatgaaatccatggtcaagaggacaagtggaaagtgagagggaaggtttgctagacaccttcgcttgttatcttgtcaagatagaaaagatagtatcatttcacccttgccagtaaaaacctttccatccacccattctcagcagactccagtattggcacagtcactcactgccattctcacactataacaagaaaagaaatgaagtgcataagtctcctgggaaaagaaccttaaccccttctcgtgccatgactggtgatttcatgactcataagcccctccgtaggcatcattcaagatcaatggcccatgcatgctgtttgcagca

SEQ ID NO: 66 - DNA, 532 bp, Unknown
  probe
aaaggccacactggtgtgcctggccacaggtatcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccatgtatgctgtgctggtcagcgcccttgtgttgatggccatggtcaagagaaaggatttctgaaggcagccctggaagtggagttaggagcttctaacccgtcatggtttcaatacacattcttcttttgccagc

SEQ ID NO: 67 - DNA, 484 bp, Unknown
  probe
ggaacaagacttcaggtcacgctcgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctgagatctgcaagattgtaagacagcctgtgctccct

SEQ ID NO: 68 - DNA, 512 bp, Unknown
  probe
aaatgatacactactgctgcagctcacaaacacctctgcatattacatgtacctcctcctgctcctcaagagtgtggtctattttgccatcatcacctgctgtctgcttggaagaacggctttctgctgcaatggagagaaatcataacagacggtggcacaaggaggccatcttttcctcatcggttattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtagacagcctgcggcttctagccttgtccctctcttagtgttctttaatcagataactgcctggaagcctttcattttacacgccc

SEQ ID NO: 69 - DNA, 483 bp, Unknown
  probe
gccatcagaagcagagatctcccacacccaaaaggccacactggtgtgcctggccacaggtttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgctgtgctggtcagtgccctcgtgctgatggccatggtcaagagaaagga

SEQ ID NO: 70 - DNA, 592 bp, unknown
  probe
gaatcgtttctctgtgaacttccagaaagcagccaaatccttcagtctcaagatctcagactcacagctgggggatgccgcgatgtatttctgtgcttataggagtgcatactctggggctgggagttaccaactcactttcgggaaggggaccaaactctcggtcataccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggttgtggtcc

SEQ ID NO: 71 - DNA, 358 bp, unknown
  misc_feature (1)...(358): n = A,T,C or G
  misc_feature 61, 95, 100, 182, 189, 218, 227, 229, 244, 258, 261,271, 302, 313, 324, 331: n = A,T,C or G
  probe
cactgcagaatgaaggaacatcccttgaggtgacccagccaacctgtggccagaaggaggnttgtaccttgaaaagacactgaaagcattttggngtgtnaagtaagggtgggcagaggaggtagaaaatcaattcaattgtcgcatcattcatggttctttaatattgatgctcagtgcantggcctnagaatatcccagcctctcttctggtttgntgagtgctntntaagtaagcatggtngaattgtttggggncanatatagtganccttggtcactggtgtttcaaacattctggnaagtcacatcnatcaagaatantttttanttttaagaaagcataaccagcaataaa

SEQ ID NO: 72 - DNA, 519 bp, Unknown
  Probe
  misc_feature (1)...(519): n = A,T,C or G
  misc_feature 284: n = A,T,C or G
tgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcncccttgtgttgatggccatggtcaagagaaaggatttctgaaggcagccctggaagtggagttaggagcttctaacccgtcatggtttcaatacacattcttcttttgccagcgcttctgaagagctgctctcacctctctgcatcccaatagatatccccctatgtgcatgcacacctgcacactcacggctgaaatctccctaacccagggggaccttagcatgcctaagtga

SEQ ID NO: 73 - DNA, 419 bp, unknown
  probe
gggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtgaacctgagactggtggcttctagaagcagccattaccaactgtacct

SEQ ID NO: 74 - DNA, 532 bp, Unknown
  Probe
  misc_feature (1)...(532): n = A,T,C or G
  misc_feature 108, 111, 121, 122, 123, 142, 171, 172, 174, 176, 187,188, 431, 433, 434: n = A,T,C or G
aaatgatacactactgctgcagctcacaaacacctctgcatattacatgtacctcctcctgctcctcaagagtgtggtctattttgccatcatcacctgctgtctgcntgnaagaacggcnnnctgctgcaatggagagaantcataacagacggtggcacaaggaggccnncntntcctcatcggnnattgtccctagaagcgtcttctgaggatctagttgggctttctttctgggtttgggccatttcagttctcatgtgtgtactattctatcattattgtataatggttttcaaaccagtgggcacacagagaacctcagtctgtaataacaatgaggaatagccatggcgatctccagcaccaatctctccatgttttccacagctcctccagccaacccaaatagcgcctgctatagtgtaganannctgcggcttctagccttgtccctctcttagtgttctttaatcagataactgcctggaagcctttcattttacacgccctgaagcagtcttctttgcta

SEQ ID NO: 75 - DNA, 267 bp, Unknown
  probe
ccaccctctggatcccaatattgagatcttatcctcagggaatcctcacttagacccctgtaacaggttaaatcttcatggtgttctgtttcctaggaacttctttcttttctactgtttatgacaactgaagttaataagtgtttatctttcccacctactcaaagtagttccaagattagggctagtttgtaattctgtggaccactgtaaacgagggcctagttcagtgtctgcctcatgggaagcttccaataaatacctttg

SEQ ID NO: 76 - DNA, 469 bp, Unknown
  probe
tctcctttctcaccaatgggcaatagcccataattgaaataaatttctgattgaaaggtataggaaacattaaaatgcattactaagagaagtaatataattttcttacaaagtatttttcccaaagatagctttactatttcaaaaattgtcaaattaatgcatgctccttacaacaaacaaatatcaaaaagagtttaggaattctactagccagagatagtcacttggagaaactttctatatatccttctaaatatttttctgggcatgctcatgtatgtacatcagttgtttctttttattttgaaccaaaaatgtggtttcttttgtacacattacttaaactttctttccagtcaacaatatattgtggatttattttcactgttatatttaactatatataaatacgcatatattgtaattttaatgtctgcttagcaccccactgataaccaaatcacag

SEQ ID NO: 77 - DNA, 97 bp, Unknown
  probe
gagagtggacatttgtcgggaaactcctaacatatgcccccattctggagagaacacagagtacgacacaatccctcacactaatagaacaatccta

SEQ ID NO: 78 - DNA, 299 bp, Unknown
  probe
aacacctgtgctaggtcagtctggcacgtaagatgaacatccctaccaacacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagta

SEQ ID NO: 79 - DNA, 413 bp, Unknown
  probe
tgaagaaagttctcctcctgatcacagccatcttggcagtggctgttggtttcccagtctctcaagaccaggaacgagaaaaaagaagtatcagtgacagcgatgaattagcttcagggttttttgtgttcccttacccatatccatttcgcccacttccaccaattccatttccaagatttccatggtttagacgtaattttcctattccaatacctgaatctgcccctacaactccccttcctagcgaaaagtaaacaagaaggaaaagtcacgataaacctggtcacctgaaattgaaattgagccacttccttgaagaatcaaaattcctgttaataaaagaaaaacaaatgtaattgaaatagcacacagcattctctagtcaatatctttagtgatcttctttaata

SEQ ID NO: 80 - DNA, 405 bp, Unknown
  probe
atgtcaggtttgtacctaccacatttaaaatagggacttgaagaattaaacattttattacaaatgaagcacttcatgcacagactggcacatagtaagtagtcgataggtgttaacaatttgtgttattgttattttctggagtccaactaacaaatcccacagtgaatgacatcacagggatgcaaccaacaagatccagaatatggaaacttctactagataaacaactccatttcttcagcaacaattcaagagagagagagaagagaagctatacattttaaaaggctgaagaaatatatgaaccaaatttgtatgaggcaatcagaaaaactgacaccgactgtattaaggaattatctaattttagtgtggtaatgagattgctgttatgttttctaa

SEQ ID NO: 81 - DNA, 365 bp, Unknown
  probe
aaaagatctctcactgggaaaagaaaaagttatgcatttataaagtaattaaactggttttccttgtactttattaatctgaatctaatggcacttccttacgagggttttcagatgtgcttgtagttaatggcaacattatcagaatgactacacagacagtcctactctgaggagatgactttggaagaaacccatttggaactacacaccctgctatgtctgtggagaaatggaactgcaatcctcaagagtcacacttcatattccttcctttcaagtggttgataaaaggtagtgcttcaagcacaggatttatggaatagttggcaaattaaacaacatgctttttattttgactacca

SEQ ID NO: 82 - DNA, 321 bp, Unknown
  Probe
  misc_feature (1)...(321): n = A,T,C or G
  misc_feature 275, 276: n = A,T,C or G
ttatcaccattaatccatgccagttatgtacagttttgcatgtttgttttttattttactcttttctctccttcattccctattcctgttcccccataggtgcccagtctaatgtatttgatatctgtccttagagcctccgtatctgtgaagactagagtcatgcacttcatcacatttcagtaacgatgggccgcatgtaccacagtcccatgagatgatagaggtgcagaaaaattcctgtcatctagtgacatcgtagccatcataacatnncaacacgactctcatttgtggtgaccctggtgtacacaaacctac

SEQ ID NO: 83 - DNA, 366 bp, Unknown
  probe
atggctggagatgtagtctacgctgacatcaaaactgttcggacttccccgttagaactcgcgtttccacttcagagatctgtttctttcaacttttctactgtccataaatcatgtcctgccaaagactggaaggtgcataagggaaaatgttactggattgctgaaactaagaaatcttggaacaaaagtcaaaatgactgtgccataaacaattcatatctcatggtgattcaagacattactgctatggtgagatttaacatttagaggtgacagcatcccccacactggcagtgaattttttgtgctacaaacttggcaaaagtctgtgaaaagaagtttcaacttcatgtgttattaact

SEQ ID NO: 84 - DNA, 548 bp, Unknown
  Probe
  misc_feature (1)...(548): n = A,T,C or G
  misc_feature 26, 43, 49, 50, 51, 370, 371, 372, 373, 374, 375, 376,377, 378, 379, 380, 381, 382, 383, 384, 385, 386, 387, 388: n = A,T,C or G
  misc_feature 468: n = A,T,C or G
taatcggctcactataggaatttgcntcgaggccaagattcgnacgagnnngttcaaaagcagctaaaccaaaagaagcctccagacagccctgagatcacctaaaaagctgctaccaagacagccacgaagatcctaccaaaatgaagcgcttcctcttcctcctactcaccatcagcctcctggttatggtacagatacaaactggactctcaggacaaaacgacaccagccaaaccagcagcccctcagcatccagcagcatgagcggaggcattttccttttcttcgtggccaatgccataatccacctcttctgcttcagttgaggtgacacgtctcagccttagccctgtgccccctgaaacannnnnnnnnnnnnnnnnnnagagaatcccctccatctttgggaggggttgatgccagacatcaccaggttgtagaagttgacaggcagtgccatggggncaacagccaaaataggggggtaatgatgtaggggccaagcagtgcccagctgggggtcaataaagttacccttgtacttg

SEQ ID NO: 85 - DNA, 388 bp, Homo Sapien; identical to SEQ ID NO: 52

SEQ ID NO: 86 - DNA, 508 bp, Homo Sapien; identical to SEQ ID NO: 54

SEQ ID NO: 87 - DNA, 501 bp, Homo Sapien; identical to SEQ ID NO: 55

SEQ ID NO: 88 - DNA, 489 bp, Homo Sapien; identical to SEQ ID NO: 56

SEQ ID NO: 89 - DNA, 405 bp, Homo Sapien; identical to SEQ ID NO: 58

SEQ ID NO: 90 - DNA, 352 bp, Homo Sapien; identical to SEQ ID NO: 65

SEQ ID NO: 91 - DNA, 484 bp, Homo Sapien; identical to SEQ ID NO: 67

SEQ ID NO: 92 - DNA, 512 bp, Homo Sapien; identical to SEQ ID NO: 68

SEQ ID NO: 93 - DNA, 483 bp, Homo Sapiien; identical to SEQ ID NO: 69

SEQ ID NO: 94 - DNA, 519 bp, Homo Sapien; identical to SEQ ID NO: 72
  misc_feature 284: n = A, T, C or G
  misc_feature 284: n = A,T,C or G

SEQ ID NO: 95 - DNA, 419 bp, Homo Sapien; identical to SEQ ID NO: 73

SEQ ID NO: 96 - DNA, 469 bp, Homo Sapien; identical to SEQ ID NO: 76

SEQ ID NO: 97 - DNA, 548 bp, Homo Sapien; identical to SEQ ID NO: 84
  misc_feature 26, 43, 49, 50, 51, 370, 371, 372, 373, 374, 375, 376,377, 378, 379, 380, 381, 382, 383, 384,  385, 386, 387, 388: n = A, T, C or G
  misc_feature 26, 43, 49, 50, 51, 370, 371, 372, 373, 374, 375, 376,377, 378, 379, 380, 381, 382, 383, 384, 385, 386, 387, 388: n = A,T,C or G
  misc_feature 468: n = A,T,C or G

SEQ ID NO: 98 - DNA, 21 bp, Unknown
  IL7R Forward primer
tcctaccagcctttgcctctt

SEQ ID NO: 99 - DNA, 23 bp, Unknown
  IL7R Reverse primer
ccatacttggcttttctgaagca

SEQ ID NO: 100 - DNA, 21 bp, Unknown
  IL7R TaqMan Probe
cttcaatgtggtttccatggg

SEQ ID NO: 101 - DNA, 25 bp, Unknown
  CD3D Forward Primers
gttgaggaatgaccaggtctatcag

SEQ ID NO: 102 - DNA, 22 bp, Unknown
  CD3D Reverse Primers
aaggtggctgtactgagcatca

SEQ ID NO: 103 - DNA, 14 bp, Unknown
  CD52 Forward Primer
cctccgagatcgag

SEQ ID NO: 104 - DNA, 23 bp, Unknown
  CD52 Forward Primer
tcagcctcctggttatggtacag

SEQ ID NO: 105 - DNA, 19 bp, Unknown
  CD52 Reverse Primer
tggtttggctggtgtcgtt

SEQ ID NO: 106 - DNA, 19 bp, Unknown
  CD52 TaqMan Probe
caaactggactctcaggac

SEQ ID NO: 107 - DNA, 20 bp, Unknown
  UBD Forward Primer
tgaggagctgcccttgtttc

SEQ ID NO: 108 - DNA, 20 bp, Unknown
  UBD Reverse Primer
ctggaggaggtgcctctttg

SEQ ID NO: 109 - DNA, 17 bp, Unknown
  UBD TaqMan Probe
catcacctgactccaca

SEQ ID NO: 110 - DNA, 25 bp, Unknown
  GPR171 Forward Primer
gaagtcataactgattgctcaacca

SEQ ID NO: 111 - DNA, 22 bp, Unknown
  GPR171 Reverse Primer
gacacagccaggagcagtgtag

SEQ ID NO: 112 - DNA, 20 bp, Unknown
  GPR171 TawMan Probe
tcactcttcaaagccaaaga

SEQ ID NO: 113 - DNA, 20 bp, Unknown
  GMZK Forward primers
gtctgtgcaggagatgccaa

SEQ ID NO: 114 - DNA, 17 bp, Unknown
  GMZK Reverse primers
gggccccctgagtcacc

SEQ ID NO: 115 - DNA, 19 bp, Unknown
  GMZK TaqMan Probe
ttacaggaatccttctggc

SEQ ID NO: 116 - DNA, 25 bp, Unknown
  PRKCQ Forward Primer
atccatggtcaagaggacaagtgga

SEQ ID NO: 117 - DNA, 24 bp, Unknown
  PRKCQ Reverse Primer
gacaagataacaagcgaaggtgtc

SEQ ID NO: 118 - DNA, 18 bp, Unknown
  PRKCQ TaqMan Probe
caaaccttccctctcact

SEQ ID NO: 119 - DNA, 22 bp, Unknown
  STAT4 Forward primer
gtcccacaacaattgaaactgc

SEQ ID NO: 120 - DNA, 24 bp, Unknown
  STAT4 Reverse primer
gtgcgtcagagtttatcctgtcat

SEQ ID NO: 121 - DNA, 20 bp, Unknown
  STAT4 TaqMan Probe
tgaagtctccttattctgct

SEQ ID NO: 122 - DNA, 23 bp, Unknown
  TRDv2 Forward primer
ggtcgagaaaagctttgaaacag

SEQ ID NO: 123 - DNA, 22 bp, Unknown
  TRDv2 Reverse primer
ggaacccaatcactgacaggtt

SEQ ID NO: 124 - DNA, 16 bp, Unknown
  TRDv2 TaqMan probe
acgaacctaaactttc

SEQ ID NO: 125 - DNA, 18 bp, Unknown
  TRAT1 forward primer
aagccaccccatctgcac

SEQ ID NO: 126 - DNA, 21 bp, Unknown
  TRAT1 reverse primer
ctgtgatcaagtgaggcgtag

SEQ ID NO: 127 - DNA, 20 bp, Unknown
  TRAT1 TawMan Probe
caaccaatgaaacacagatg

SEQ ID NO: 128 - DNA, 19 bp, Unknown
  TRBV19 Forward primer
gaggcctggggtagagcag

SEQ ID NO: 129 - DNA, 20 bp, Unknown
  TRBV19 Reverse primer
agaggatggtggcagacagg

SEQ ID NO: 130 - DNA, 17 bp, Unknown
  TRBV19 TaqMan probe
tgtcctaccagcaaggg

SEQ ID NO: 131 - DNA, 26 bp, Unknown
  CD69 Foward Primer
aatcatagctctcattgccttatcag

SEQ ID NO: 132 - DNA, 23 bp, Unknown
  CD69 Reverse Primer
tgtctgatggcattgagaatgtg

SEQ ID NO: 133 - DNA, 20 bp, Unknown
  CD69 TaqMan Probe
ccaatacaattgtccaggcc

SEQ ID NO: 134 - DNA, 19 bp, Unknown
  INDO1 Forward primer
ccagcagactgctggtgga

SEQ ID NO: 135 - DNA, 25 bp, Unknown
  INDO1 Reverse primer
ggcatatatcttctcatgtcctgga

SEQ ID NO: 136 - DNA, 16 bp, Unknown
  INDO1 TaqMan Probe
acatgctgctcagttc

SEQ ID NO: 137 - DNA, 20 bp, Unknown
  H3F3A Forward primer
cagctatcggtgctttgcag

SEQ ID NO: 138 - DNA, 24 bp, Unknown
  H3F3A Reverse primer
gatagcacacaggttggtgtcttc

SEQ ID NO: 139 - DNA, 16 bp, Unknown
  H3F3A TaqMan Probe
caagtgaggcctatct

SEQ ID NO: 140 - DNA, 25 bp, Unknown
  CD45R Forward primer
gagatgcctgatggttcaagtagag

SEQ ID NO: 141 - DNA, 25 bp, Unknown
  CD45R Reverse primer
ttcttcaaactgattgtattccacc

SEQ ID NO: 142 - DNA, 19 bp, Unknown
  CD45R TaqMan Probe
cagtacatcttgatccatc

SEQ ID NO: 143 - DNA, 19 bp, Unknown
  CD45RO Forward primer
ggcaaagcccaacaccttc

SEQ ID NO: 144 - DNA, 22 bp, Unknown
  CD45RO Reverse primer
cagaagggctcagagtggttgt

SEQ ID NO: 145 - DNA, 16 bp, Unknown
  CD45RO TaqMan probe
tgcctaccttaatgcc

SEQ ID NO: 146 - DNA, 19 bp, Unknown
  FoxP3 Forward primer
cacctggctgggaaaatgg

SEQ ID NO: 147 - DNA, 19 bp, Unknown
  FoxP3 Reverse primer
ggagcccttgtcggatgat

SEQ ID NO: 148 - DNA, 16 bp, Unknown
  FoxP3 TaqMan Probe
caaggcttcatctgtg

SEQ ID NO: 149 - DNA, 25 bp, Unknown
  CD20 Forward primer
catacaatctctgttcttgggcatt

SEQ ID NO: 150 - DNA, 22 bp, Unknown
  CD20 Reverse primer
tcattctcaacgatgccagcta

SEQ ID NO: 151 - DNA, 17 bp, Unknown
  CD20 TaqMan Probe
tcagtgatgctgatctt

SEQ ID NO: 152 - DNA, 19 bp, Unknown
  TLR9 Forward primer
cgccagaccctctggagaa

SEQ ID NO: 153 - DNA, 19 bp, Unknown
  TLR9 Reverse primer
gcctgcaccaggagagaca

SEQ ID NO: 154 - DNA, 13 bp, Unknown
  TLR9 TaqMan Probe
ttctgccgcagcg

SEQ ID NO: 155 - DNA, 27 bp, Unknown
  ITGB3 Forward Primer
aagtccatcctgtatgtggtagaagag

SEQ ID NO: 156 - DNA, 20 bp, Unknown
  ITGB3 Reverse Primer
gagcaggaccaccaggatgt

SEQ ID NO: 157 - DNA, 14 bp, Unknown
  ITGB3 TaqMan Probe
agtgtcccaagggc

SEQ ID NO: 158 - DNA, 21 bp, Unknown
  TlR3 Forward primer
ggaaaggctagcagtcatcca

SEQ ID NO: 159 - DNA, 20 bp, Unknown
  TlR3  Reverse  primer
gtggaggatgcacacagcat

SEQ ID NO: 160 - DNA, 17 bp, Unknown
  TLR3 Taqman Probe
atgagacagactttgcc

SEQ ID NO: 161 - DNA, 19 bp, Unknown
  TLR4 Forward Primer
ccagaactgcaggtgctgg

SEQ ID NO: 162 - DNA, 22 bp, Unknown
  TLR4 Reverse Primer
ggtggcttaggctctgatatgc

SEQ ID NO: 163 - DNA, 20 bp, Unknown
  TLR4 TaqMan Probe
ccaggtgtgaaatccagaca